(19) United States
(12) Patent Application Publication
(10) Pub. No.: US 2023/0079833 A1
(43) Pub. Date: Mar. 16, 2023

(54) GALACTOSE-LINKED MULTIMERIC GLYCOMIMETIC INHIBITORS OF E-SELECTINS, GALECTIN-3, AND/OR CXCR4 CHEMOKINE RECEPTORS

(71) Applicant: GLYCOMIMETICS, INC., Rockville, MD (US)

(72) Inventors: John L. MAGNANI, Gaithersburg, MD (US); John M. PETERSON, Slate Hill, NY (US); Arun K. SARKAR, North Potomac, MD (US); Yusufbhai U. VOHRA, Germantown, MD (US); Myung-Gi BAEK, Boyds, MD (US); Hong-Woon YANG, Marlborough, MA (US)

(73) Assignee: GLYCOMIMETICS, INC., Rockville, MD (US)

(21) Appl. No.: 17/605,107

(22) PCT Filed: Apr. 21, 2020
(86) PCT No.: PCT/US2020/029066

§ 371 Date: Oct. 20, 2021

Related U.S. Application Data
(60) Provisional application No. 62/838,206, filed on Apr. 24, 2019.

(51) Int. Cl. A61K 47/55; A61K 45/06; A61P 1/04; A61P 35/00; C07H 15/207; C07H 15/22; C07H 19/056

(52) U.S. Cl. CPC A61K 47/55; A61K 45/06; A61P 1/04; C07H 19/056; C07H 15/207; C07H 15/22; A61P 35/00

(57) ABSTRACT

Compounds, compositions, and methods for treating and/or preventing at least one disease, disorder, and/or condition N associated with E-selectin, galectin-3, and/or CXCR4 chemokine receptor activity are disclosed herein. For example, multimeric glycomimetic inhibitors of E-selectins, galectin-3, and/or CXCR4 chemokine receptors and their use for treating and/or preventing inflammatory diseases, fibrosis, and cancers are disclosed. Formula (I).

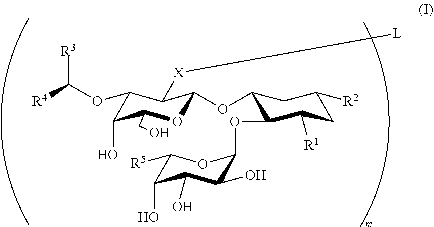

This application claims the benefit under 35 U.S.C. § 119(e) to U.S. Provisional Application No. 62/838,206 filed Apr. 24, 2019, which application is incorporated by reference herein in its entirety.

Compounds, compositions, and methods for treating and/or preventing at least one disease, disorder, and/or condition associated with E-selectin, galectin-3, and/or CXCR4 chemokine receptor activity are disclosed herein.

A number of cancers are treatable before the cancer has moved beyond the primary site. However, once the cancer has spread beyond the primary site, the treatment options may be limited and the survival statistics may decline dramatically. Bones are a common location for cancer to infiltrate once leaving the primary tumor location. Breast and prostate cancer are examples of cancers that migrate to bones. Even leukemic cells that arise in the bloodstream may home to the bone marrow. Once cancer resides in bone, it may cause pain in an individual. Furthermore, once in the bone marrow, the cancer cells may also become resistant to chemotherapy. In addition, if the particular bone affected produces blood cells in the bone marrow, the individual may develop a variety of blood cell related disorders. Thus, it may be desirable to prevent cancer cells from leaving the primary site and/or to prevent extravasation of cancer cells from the bloodstream and infiltration into other tissues. Retention of cancer cells in the bloodstream makes the cells more susceptible to treatment, such as chemotherapy.

Some cancers originate all or in part in bone. For such cancers, it may be desirable to mobilize cancer cells from bone to the bloodstream and/or to prevent those cells (as well as any cancer cells already in the bloodstream) from homing to bone or otherwise leaving the bloodstream. Retention of cancer cells in the bloodstream (or mobilization of cancer cells into the bloodstream and then retention therein) makes the cells more susceptible to treatment, such as chemotherapy.

Hematopoietic stem cells (HSCs) also reside in the bone marrow and are a source of material for cellular therapy. HSCs adhere to the stroma within the bone marrow and in order to be harvested must break these adhesions and mobilize out of the bone marrow. Improved agents for increasing the number of HSCs available for harvesting may be desirable. Such HSCs may be useful for engraftment.

When a tissue is infected or damaged, the inflammatory process directs leukocytes and other immune system components to the site of infection or injury. Within this process, leukocytes play an important role in the engulfment and digestion of microorganisms. The recruitment of leukocytes to infected or damaged tissue is critical for mounting an effective immune defense.

Selectins are a group of structurally similar cell surface receptors important for mediating leukocyte binding to endothelial cells. These proteins are type 1 membrane proteins and are composed of an amino terminal lectin domain, an epidermal growth factor (EGF)-like domain, a variable number of complement receptor related repeats, a hydrophobic domain spanning region and a cytoplasmic domain. The binding interactions appear to be mediated by contact of the lectin domain of the selectins and various carbohydrate ligands.

There are three known selectins: E-selectin, P-selectin, and L-selectin. E-selectin is found on the surface of activated endothelial cells, which line the interior wall of capillaries. E-selectin binds to the carbohydrate sialyl-Lewis^{x} (sLe^{x}), which is presented as a glycoprotein or glycolipid on the surface of certain leukocytes (monocytes and neutrophils) and helps these cells adhere to capillary walls in areas where surrounding tissue is infected or damaged; and E-selectin also binds to sialyl-Lewis^{a} (sLe^{a}), which is expressed on many tumor cells P-selectin is expressed on inflamed endothelium and platelets, and also recognizes sLe^{x} and sLe^{a}, but also contains a second site that interacts with sulfated tyrosine. The expression of E-selectin and P-selectin is generally increased when the tissue adjacent to a capillary is infected or damaged. L-selectin is expressed on leukocytes. Selectin-mediated intercellular adhesion is an example of a selectin-mediated function.

Although selectin-mediated cell adhesion is required for fighting infection and destroying foreign material, there are situations in which such cell adhesion is undesirable or excessive, resulting in tissue damage instead of repair. For example, many pathologies (such as autoimmune and inflammatory diseases, shock and reperfusion injuries) involve abnormal adhesion of white blood cells. Such abnormal cell adhesion may also play a role in transplant and graft rejection. In addition, some circulating cancer cells appear to take advantage of the inflammatory mechanism to bind to activated endothelium and metastasize. In such circumstances, modulation of selectin-mediated intercellular adhesion may be desirable.

E-selectin inhibitors are known in the art. Some E-selectin inhibitors are specific for E-selectin only. Other E-selectin inhibitors have the ability to inhibit not only E-selectin but additionally P-selectin or L-selectin or both P-selectin and L-selectin. Examples of E-selectin inhibitors (specific for E-selectin or otherwise) are disclosed in U.S. Pat. No. 7,060,685; U.S. Application Publication No. US-2007-0054870; U.S. Application Publication No. US-2008-0161546; and references cited in any of these patents or published applications. Those examples are small organic molecules. Other known E-selectin inhibitors are amino acid-based, such as antibodies. For example, the humanized monoclonal antibody CDP850 is an E-selectin inhibitor.

Galectins are proteins with a characteristic carbohydrate recognition domain (CRD) (Barondes, S. H., Cooper, D. N. W., Gitt, M. A., and Leffler, H. (1994). Galectins. Structure and function of a large family of animal lectins. J. Biol. Chem. 269:20807-20810; Leffler, H., Carlsson, S., Hedlund, M., Qian, Y. and Poirier, F. (2004) Introduction to galectins. Glycoconj. J. 19; 433-440). Galectin subunits can contain either one or two CRDs within a single peptide chain. The mono-CRDs galectins can occur as monomers or dimers in verterates. Galectin-3 is a monomer in solution but may aggregate and become multimeric upon encounter with ligands. Galectins are synthesized as cytosolic proteins. Evidence suggests roles for galectins in inflammation, fibrosis, cancer, and other disorders (see, e.g., U.S. Pat. No. 7,638,623).

A pro-inflammatory role of galectin-3 is indicated by its induction in cells at inflammatory sites, effects on immune cells, and decrease of the inflammatory response shown in animal models. Inflammation is a protective response of the body to invading organisms and tissue injury. However, if unbalanced, frequently it is also destructive and occurs as part of the pathology in many diseases. Because of this, there is great medical interest in pharmacological modulation of galectin-3 mediated inflammation.

Immunohistochemical studies show changed expression of certain galectins in cancer. Direct evidence for a role of galectin-3 in cancer comes from mouse models. In paired tumor cell lines (with decreased or increased expression of galectin-3), the induction of galectin-3 gives more tumors and metastasis and suppression of galectin-3 gives less tumors and metastasis. Galectin-3 has been proposed to enhance tumor growth by being anti-apoptotic, promote angiogenesis, or to promote metastasis by affecting cell adhesion.

Both natural and synthetic modulators of galectin-3 have been identified. However, natural compounds that have been identified as galectin-3 ligands are not suitable for use as active components in pharmaceutical compositions, because they have been reported to have low activity and specificity for galectins and galectin-3. As natural products they are difficult to produce as well-characterized drugs and are susceptible to acidic hydrolysis in the stomach and to enzymatic degradation. In addition, previously identified natural galectin-3 modulators are large and hydrophilic in nature, and are not readily absorbed from the gastrointestinal tract following oral administration.

CXCR4 is a G-protein-coupled receptor that is expressed by both mononuclear and progenitor cells in the bone marrow. The ligand for CXCR4, stromal derived factor-1 (SDF-1), is a secreted or membrane-bound protein that is abundantly expressed in the osteoblast and vascular niches. SDF-1/CXCR4 signaling induces the directional migration of cells and is involved in a large number of physiological processes including inflammation, cancer, stem cell migration, HIV, and cell migration. (Cheng et al., Prog. MolBiol Trans/Sci., 111:243-264, 2012.)

CXCR4 chemokine receptor inhibitors are known in the art. Such inhibitors will typically prevent the binding of SDF-1 to a CXCR4 receptor. Examples of CXCR4 chemokine receptor inhibitors are AMD-3100 (Hendrix et al., Antimicrob. Agents Chemother. 44:1667-1673, 2000); ALX40-4C (Doranz et al., AIDS Research and Human Retroviruses 17:475-486, 2001); and T134 (Arakaki et al., J. Virol. 73:1719-1723, 1999). These examples include a small organic molecule and amino acid-based molecules, such as the T22 peptide. AMD-3100 is a bicyclam. Each of the two cyclam rings is attached to the same phenyl ring (each cyclam ring is para to the other) via a methylene group.

Accordingly, there is a need in the art for inhibitors of E-selectin, galectin-3, or CXCR4 chemokine receptor activity, or combinations thereof, and for the development of methods employing such compounds. The present disclosure may fulfill one or more of these needs and/or may provide other advantages. For example, the compounds of the present disclosure may be highly potent E-selectin, galectin-3, and/or CXCR4 chemokine receptor antagonists.

Compounds, compositions, and methods for treating and/or preventing (i.e., reducing the likelihood of occurrence or reoccurrence) at least one disease, disorder, and/or condition in which inhibiting binding of E-selectin, galectin-3, and/or CXCR4 chemokine receptors to one or more ligands may play a role are disclosed. Compounds disclosed herein are multimeric glycomimetic modulators of E-selectins, galectin-3, and/or CXCR4 chemokine receptors.

Disclosed are multimeric glycomimetic inhibitors of Formula (I):

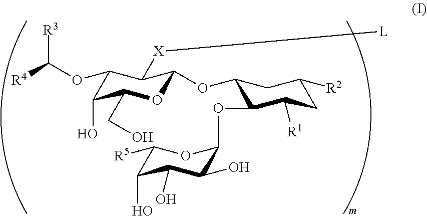

prodrugs of Formula (I), and pharmaceutically acceptable salts of any of the foregoing, wherein R¹, R², R³, R⁴, R⁵, X, L, and m are defined herein.

As used herein, ‘compound of Formula (I)’ includes multimeric glycomimetic inhibitors of Formula (I), pharmaceutically acceptable salts of multimeric glycomimetic inhibitors of Formula (I), prodrugs of multimeric glycomimetic inhibitors of Formula (I), and pharmaceutically acceptable salts of prodrugs of multimeric glycomimetic inhibitors of Formula (I).

In some embodiments, pharmaceutical compositions comprising at least one compound of Formula (I) and optionally at least one additional pharmaceutically acceptable ingredient are presented.

In some embodiments, a method for treatment and/or prevention of at least one disease, disorder, and/or condition where inhibition of E-selectin, galectin-3, CXCR4 chemokine receptor and mediated functions, or any combination thereof, is useful is disclosed, the method comprising administering to a subject in need thereof an effective amount of at least one compound of Formula (I) and/or a pharmaceutical composition comprising at least one compound of Formula (I).

In the following description, certain specific details are set forth in order to provide a thorough understanding of various embodiments. However, one skilled in the art will understand that the disclosed embodiments may be practiced without these details. In other instances, well-known structures have not been shown or described in detail to avoid unnecessarily obscuring descriptions of the embodiments. These and other embodiments will become apparent upon reference to the following detailed description and attached drawings.

### BRIEF DESCRIPTION OF THE DRAWINGS

FIG. 1 is a diagram illustrating the prophetic synthesis of compound 11.

FIG. 2 is a diagram illustrating the prophetic synthesis of compound 14.

FIG. 3 is a diagram illustrating the prophetic synthesis of multimeric compounds 21 and 22.

FIG. 4 is a diagram illustrating the prophetic synthesis of multimeric compounds 36 and 37.

FIG. 5 is a diagram illustrating the prophetic synthesis of multimeric compounds 44, 45, and 46.

FIG. 6 is a diagram illustrating the prophetic synthesis of multimeric compounds 55 and 56.

FIG. 7 is a diagram illustrating the prophetic synthesis of compound 60.

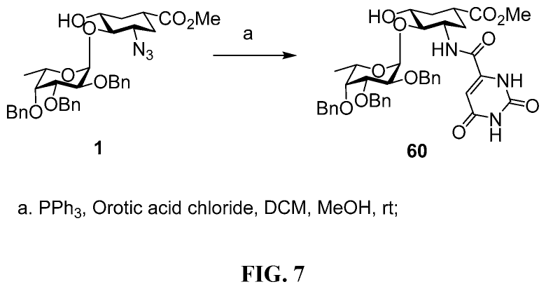

FIG. 8 is a diagram illustrating the prophetic synthesis of compound 65.

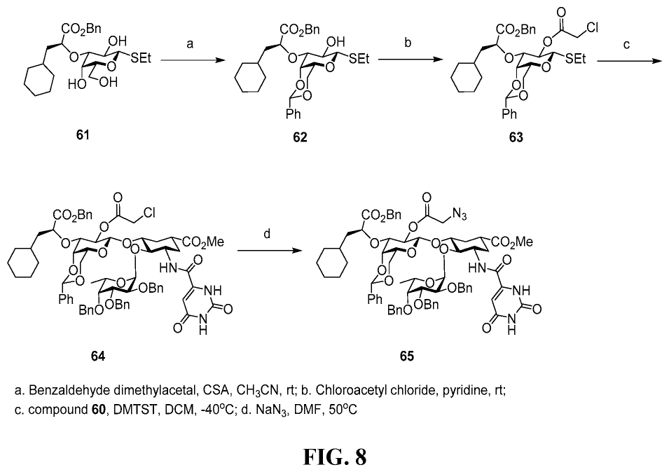

FIG. 9 is a diagram illustrating the prophetic synthesis of multimeric compounds 66, 67, and 68.

FIG. 10 is a diagram illustrating the prophetic synthesis of multimeric compounds 72 and 73.

FIG. 11 is a diagram illustrating the prophetic synthesis of multimeric compounds 76, 77, and 78.

FIG. 12 is a diagram illustrating the prophetic synthesis of multimeric compounds 86 and 87.

FIG. 13 is a diagram illustrating the prophetic synthesis of multimeric compound 95.

FIG. 14 is a diagram illustrating the prophetic synthesis of multimeric compound 146.

FIG. 15 is a diagram illustrating a prophetic synthesis of multimeric compound 197.

FIG. 16 is a diagram illustrating a synthesis of compound 205.

FIG. 17 is a diagram illustrating the synthesis of multimeric compound 206.

FIG. 18 is a diagram illustrating the synthesis of compound 214.

FIG. 19 is a diagram illustrating the synthesis of multimeric compounds 218, 219, and 220.

FIG. 20 is a diagram illustrating the synthesis of multimeric compound 224.

FIG. 21 is a diagram illustrating the prophetic synthesis of compound 237.

FIG. 22 is a diagram illustrating the prophetic synthesis of compound 241.

FIG. 23 is a diagram illustrating the prophetic synthesis of compound 245.

FIG. 24 is a diagram illustrating the prophetic synthesis of multimeric compound 257.

FIG. 25 is a diagram illustrating the prophetic synthesis of multimeric compounds 261, 262, and 263.

FIG. 26 is a diagram illustrating the prophetic synthesis of multimeric compounds 274, 275, and 276.

FIG. 27 is a diagram illustrating the prophetic synthesis of compound 291.

FIG. 28 is a diagram illustrating the prophetic synthesis of multimeric compounds 294 and 295.

FIG. 29 is a diagram illustrating the prophetic synthesis of multimeric compounds 305, 306, and 307.

FIG. 30 is a diagram illustrating the synthesis of compound 316.

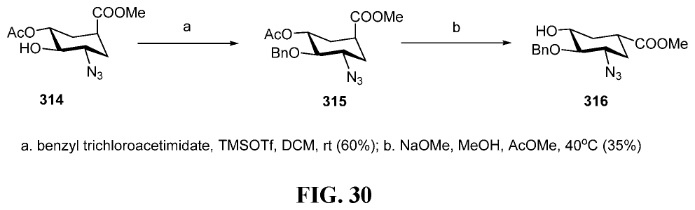

FIG. 31 is a diagram illustrating the synthesis of compound 318.

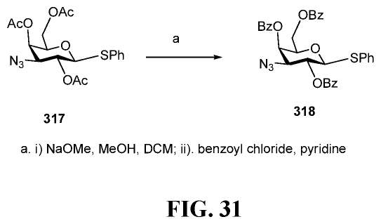

FIG. 32 is a diagram illustrating the synthesis of compound 145.

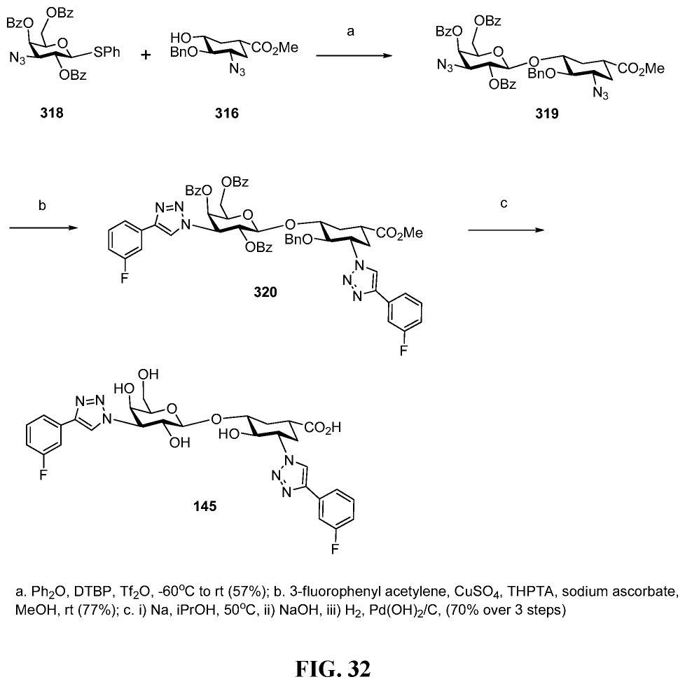

FIG. 33 is a diagram illustrating the synthesis of compound 332.

Disclosed herein are multimeric glycomimetic antagonists, pharmaceutical compositions comprising the same, and methods for inhibiting E-selectin, galectin-3, and/or CXCR4 chemokine receptor mediated functions using the same. The compounds and compositions of the present disclosure may be useful for treating and/or preventing at least one disease, disorder, and/or condition that is treatable by inhibiting binding of E-selectin, galectin-3, and/or CXCR4 chemokine receptors to one or more ligands.

The compounds of the present disclosure may have at least one improved physicochemical, pharmacological, and/or pharmacokinetic property.

In some embodiments, presented are multimeric glycomimetic antagonists of Formula (I):

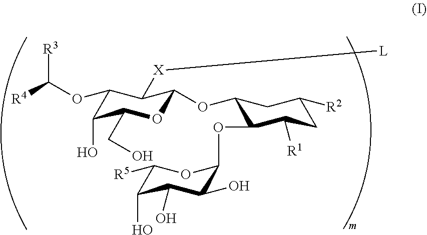

prodrugs of Formula (I), and pharmaceutically acceptable salts of any of the foregoing, wherein

each R¹, which may be identical or different, is independently chosen from H, C₁₋₁₂ alkyl, C₂₋₁₂ alkenyl, C₂₋₁₂ alkynyl, C₁₋₈ haloalkyl, C₂₋₈ haloalkenyl, C₂₋₈ haloalkynyl,

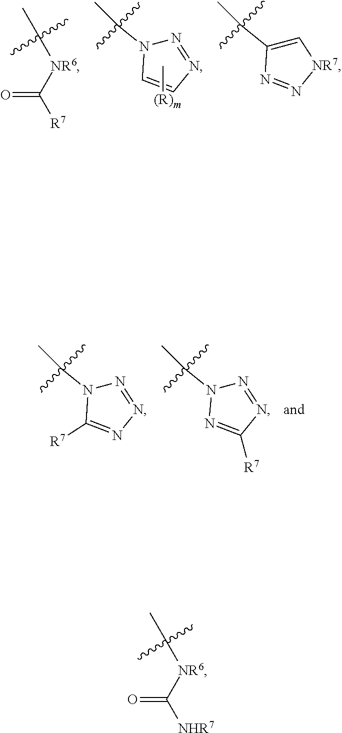

groups, wherein each n, which may be identical or different, is independently chosen from integers ranging from 0 to 2, each R⁶, which may be identical or different, is independently chosen from H, C₁₋₈ alkyl, C₂₋₈ alkenyl, C₂₋₈ alkynyl, C₄₋₁₆ cycloalkylalkyl, and —C(═O)R⁷ groups, and each R⁷, which may be identical or different, is independently chosen from H, C₁₋₈ alkyl, C₂₋₈ alkenyl, C₂₋₉ alkynyl, C₄₋₁₆ cycloalkylalkyl, C₆₋₁₈ is aryl, and C₁₋₁₃ heteroaryl groups;

each R², which may be identical or different, is independently chosen from H, a non-glycomimetic moiety, and a linker-non-glycomimetic moiety, wherein each non-glycomimetic moiety, which may be identical or different, is independently chosen from galectin-3 inhibitors, CXCR4 chemokine receptor inhibitors, polyethylene glycol, thiazolyl, chromenyl, C₁₋₈ alkyl, R⁸, C₆₋₁₈ aryl-R⁸, C₁₋₁₂ heteroaryl-R⁸,

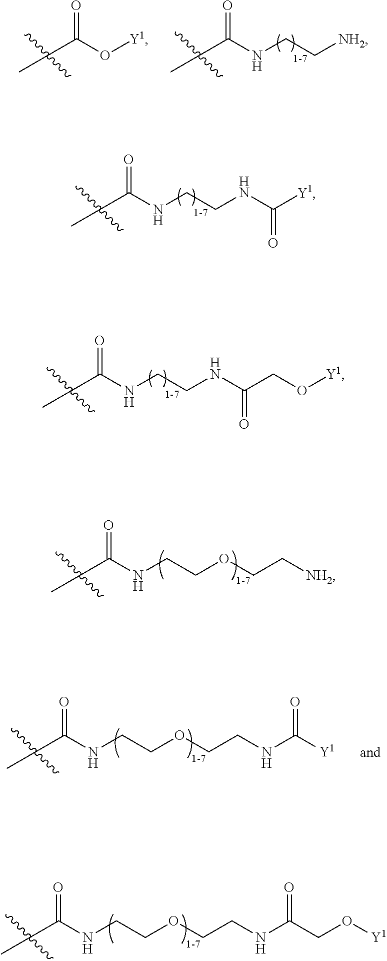

groups,
wherein each Y¹, which may be identical or different, is independently chosen from C₁₋₄ alkyl, C₂₋₄ alkenyl, and C₂₋₄ alkynyl groups and wherein each R⁸, which may be identical or different, is independently chosen from C₁₋₁₂ alkyl groups substituted with at least one substituent chosen from —OH, —OSO₃Q, —OPO₃Q₂, —CO₂Q, and —SO₃Q groups and C₂₋₁₂ alkenyl groups substituted with at least one substituent chosen from —OH, —OSO₃Q, —OPO₃Q₂, —CO₂Q, and —SO₃Q groups, wherein each Q, which may be identical or different, is independently chosen from H and pharmaceutically acceptable cations;

each R³, which may be identical or different, is independently chosen from —CN, —CH₂CN, and —C(═O)Y² groups, wherein each Y², which may be identical or different, is independently chosen from C₁₋₈ alkyl, C₂₋₈ alkenyl, C₂₋₁₂ alkynyl, —OZ¹, —NHOH, —NHOCH₃, —NHCN, and —NZ¹Z² groups, wherein each Z¹ and Z², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, and C₇₋₁₂ arylalkyl groups, wherein Z¹ and Z² may join together along with the nitrogen atom to which they are attached to form a ring;

each R⁴, which may be identical or different, is independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₄₋₁₆ cycloalkylalkyl, and C₆₋₁₈ aryl groups;
each R⁵, which may be identical or different, is independently chosen from —CN, C₁₋₁₂ alkyl, and C₁₋₁₂ haloalkyl groups;
each X, which may be identical or different, is independently chosen from —O— and —N(R⁹)—, wherein each R⁹, which may be identical or different, is independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups.

m is chosen from integers ranging from 2 to 256; and

L is independently chosen from linker groups.

In some embodiments, at least one R¹ is chosen from H, C₁₋₁₂ alkyl, and C₁₋₁₂ haloalkyl groups. In some embodiments, at least one R¹ is chosen from H and C₁₋₈ alkyl groups. In some embodiments, at least one R¹ is H. In some embodiments, at least one R¹ is chosen from C₁₋₆ alkyl groups. In some embodiments, at least one R¹ is chosen from C₁₋₄ alkyl groups. In some embodiments, at least one R¹ is chosen from methyl and ethyl. In some embodiments, at least one R¹ is methyl. In some embodiments, at least one R¹ is ethyl.

In some embodiments, at least one R¹ is chosen from

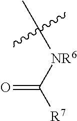

groups.

In some embodiments, at least one R¹ is chosen from

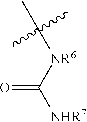

groups.

In some embodiments, at least one R¹ is chosen from

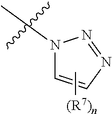

groups.

In some embodiments, at least one R⁶ is chosen from H, C₁₋₈ alkyl, and —C(═O)R⁷ groups. In some embodiments, at least one R⁶ is chosen from H and C₁₋₈ alkyl groups. In some embodiments, at least one R⁶ is chosen from C₁₋₄ alkyl groups. In some embodiments, at least one R⁶ is H.

In some embodiments, at least one R⁷ is chosen from H, C₁₋₈ alkyl, C₆₋₁₈ aryl groups, and C₁₋₁₃ heteroaryl groups. In some embodiments, at least one R⁷ is chosen from C₁₋₈ alkyl groups. In some embodiments, at least one R⁷ is chosen from C₁₋₄ alkyl groups. In some embodiments, at least one R⁷ is chosen from methyl and ethyl. In some embodiments, at least one R⁷ is H. In some embodiments, at least one R⁷ is methyl. In some embodiments, at least one R⁷ is ethyl.

In some embodiments, at least one R⁷ is chosen from

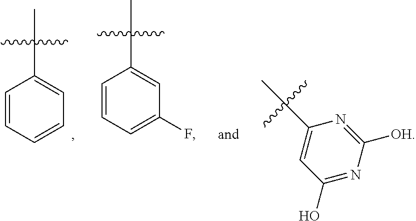

In some embodiments, at least one R¹ is

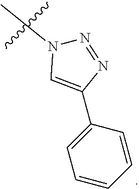 [SMILES: *n1cc(-c2ccccc2)nn1]

In some embodiments, at least one R¹ is

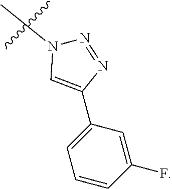 [SMILES: *n1cc(-c2cccc(F)c2)nn1]

In some embodiments, at least one R¹ is

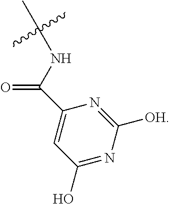 [SMILES: *NC(=O)c1cc(O)nc(O)n1]

In some embodiments, each R¹, which may be identical or different, is independently chosen from H, C₁₋₁₂ alkyl, and C₁₋₁₂ haloalkyl groups. In some embodiments, each R¹, which may be identical or different, is independently from H and C₁₋₈ alkyl groups. In some embodiments, each R¹, which may be identical or different, is independently chosen from C₁₋₆ alkyl groups. In some embodiments, each R¹, which may be identical or different, is independently chosen from C₁₋₄ alkyl groups. In some embodiments, each R¹, which may be identical or different, is independently chosen from methyl and ethyl.

In some embodiments, each R¹, which may be identical or different, is independently chosen from

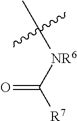

groups.

In some embodiments, each R¹, which may be identical or different, is independently chosen from

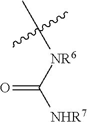

groups.

In some embodiments, each R¹, which may be identical or different, is independently chosen from

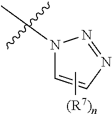

groups.

In some embodiments, each R⁶, which may be identical or different, is independently chosen from H, C₁₋₈ alkyl, and —C(═O)R⁷ groups. In some embodiments, each R⁶, which may be identical or different, is independently chosen from H and C₁₋₈ alkyl groups. In some embodiments, each R⁶, which may be identical or different, is independently chosen from C₁₋₄ alkyl groups.

In some embodiments, each R⁷, which may be identical or different, is independently chosen from H, C₁₋₈ alkyl, C₆₋₁₈ aryl groups, and C₁₋₃ heteroaryl groups. In some embodiments, each R⁷, which may be identical or different, is independently chosen from C₁₋₈ alkyl groups. In some embodiments, each R⁷, which may be identical or different, is independently chosen from C₁₋₄ alkyl groups. In some embodiments, each R⁷, which may be identical or different, is independently chosen from methyl and ethyl.

In some embodiments, each R⁷, which may be identical or different, is independently chosen from

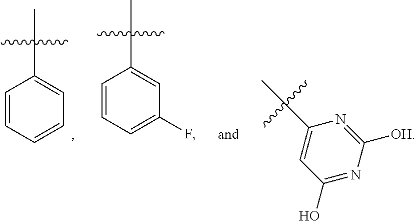

In some embodiments, each R¹ is identical and chosen from H, C₁₋₁₂ alkyl, and C₁₋₁₂ haloalkyl groups. In some embodiments, each R¹ is identical and chosen from H and C₁₋₈ alkyl groups. In some embodiments, each R¹ is H. In some embodiments, each R¹ is identical and chosen from C₁₋₆ alkyl groups. In some embodiments, each R¹ is identical and chosen from C₁₋₄ alkyl groups. In some embodiments, each R¹ is identical and chosen from methyl and ethyl. In some embodiments, each R¹ is methyl. In some embodiments, each R¹ is ethyl.

In some embodiments, each R¹ is identical and chosen from

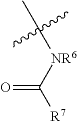

groups.

In some embodiments, each R¹ is identical and chosen from

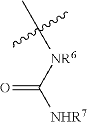

groups.

In some embodiments, each R¹ is identical and chosen from

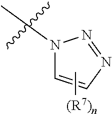

groups.

In some embodiments, each R⁶ is identical and chosen from H, C₁₋₈ alkyl, and —C(═O)R⁷ groups. In some embodiments, each R⁶ is identical and chosen from H and C₁₋₈ alkyl groups. In some embodiments, each R⁶ is identical and chosen from C₁₋₄ alkyl groups. In some embodiments, each R⁶ is H.

In some embodiments, each R⁷ is identical and chosen from H, C₁₋₈ alkyl, C₆₋₁₈ aryl groups, and C₁₋₁₃ heteroaryl groups. In some embodiments, each R⁷ is identical and chosen from C₁₋₈ alkyl groups. In some embodiments, each R⁷ is identical and chosen from C₁₋₄ alkyl groups. In some embodiments, each R⁷ is identical and chosen from methyl and ethyl. In some embodiments, each R is H. In some embodiments, each R is methyl. In some embodiments, each R⁷ is ethyl.

In some embodiments, each R⁷ is identical and chosen from

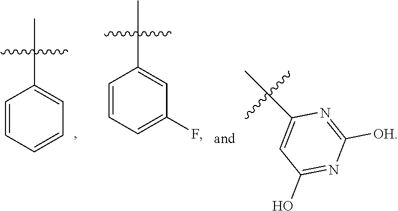 [SMILES: *c1ccccc1 ; *c1cccc(F)c1 ; *c1cc(O)nc(O)n1]

In some embodiments, each R¹ is

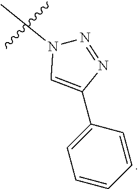 [SMILES: *n1cc(-c2ccccc2)nn1]

In some embodiments, each R¹ is

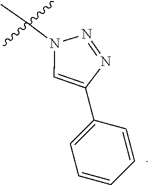 [SMILES: *n1cc(-c2ccccc2)nn1]

In some embodiments, each R¹ is

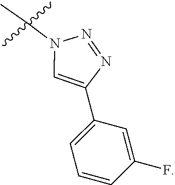 [SMILES: *n1cc(-c2cccc(F)c2)nn1]

In some embodiments, at least one R² is H. In some embodiments, each R² is H.

In some embodiments, at least one R² is chosen from

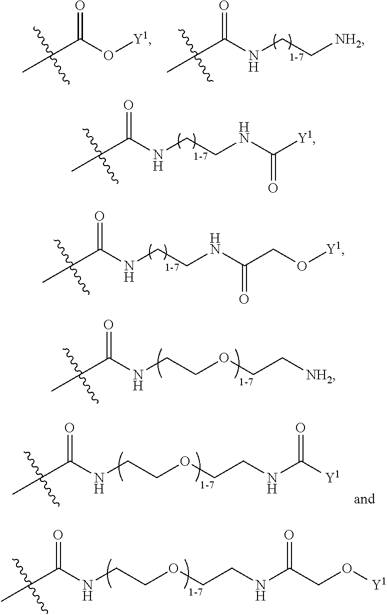

groups.

In some embodiments, at least one R² is chosen from

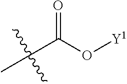

groups.

In some embodiments, at least one R² is

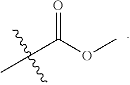 [SMILES: *C(=O)OC]

In some embodiments, at least one R² is chosen from

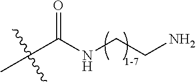

groups.

In some embodiments, at least one R² is chosen from

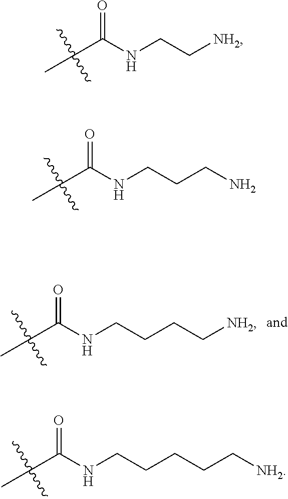 [SMILES: *C(=O)NCCN ; *C(=O)NCCCN ; *C(=O)NCCCCN ; *C(=O)NCCCCCN]

In some embodiments, at least one R² is chosen from

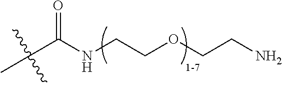

groups.

In some embodiments, at least one R² is chosen from

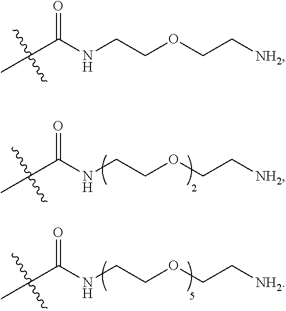

In some embodiments, at least one R² is chosen from

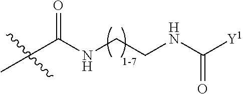

groups.

In some embodiments, at least one R² is

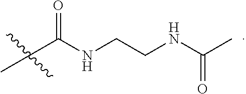 [SMILES: *C(=O)NCCNC(C)=O]

In some embodiments, at least one R² is chosen from

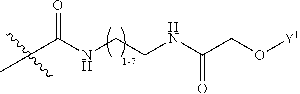

groups.

In some embodiments, at least one R² is

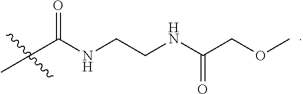 [SMILES: *C(=O)NCCNC(=O)COC]

In some embodiments, at least one Y is chosen from C₁₋₄ alkyl groups. In some embodiments, at least one Y¹ is methyl.

In some embodiments, each R², which may be identical or different, is independently chosen from

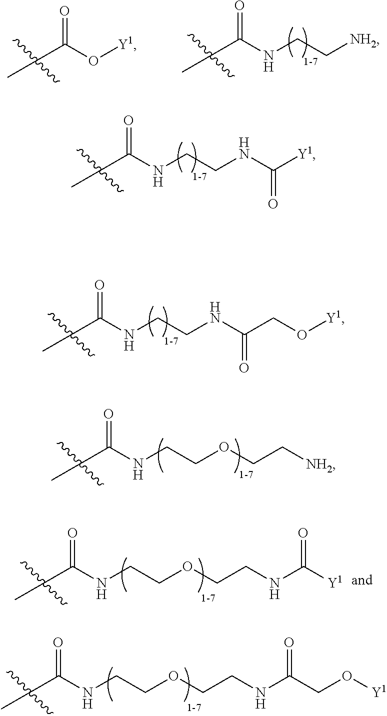

groups.

In some embodiments, each R², which may be identical or different, is independently chosen from

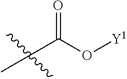

groups.

In some embodiments, each R², which may be identical or different, is independently chosen from

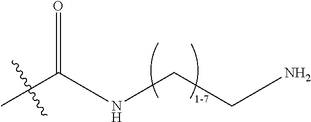

groups.

In some embodiments, each R², which may be identical or different, is independently chosen from

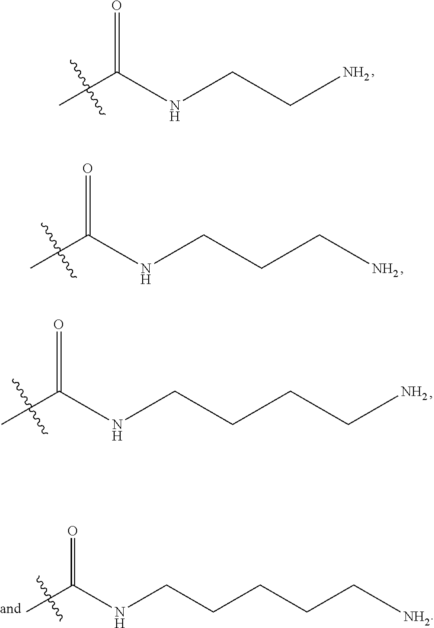 [SMILES: *C(=C=O)CNCCCCCN ; *C(=C=O)CNCCCCCCCN ; *C(=C=O)CNCCCCCCCCCN ; *C(=C=O)CNCCCCCCCCCCCN]

In some embodiments, each R², which may be identical or different, is independently chosen from

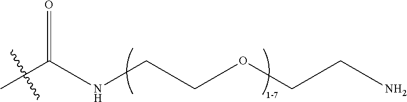

groups.

In some embodiments, each R², which may be identical or different, is independently chosen from

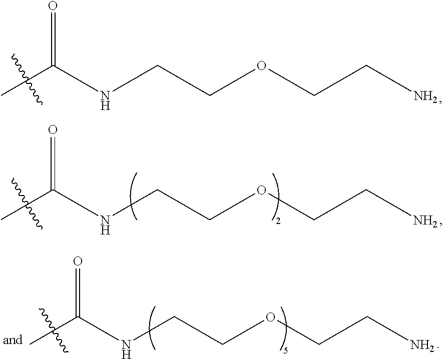

In some embodiments, each R², which may be identical or different, is independently chosen from

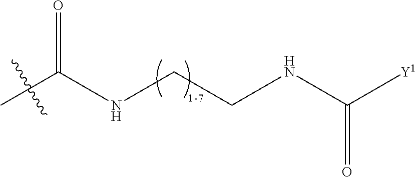

groups.

In some embodiments, each R², which may be identical or different, is independently chosen from

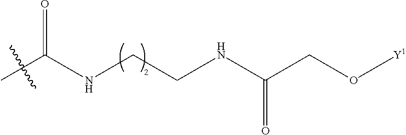

groups.

In some embodiments, each Y¹, which may be identical or different, is independently chosen from C₁₋₄ alkyl groups.

In some embodiments, each R² is identical and chosen from

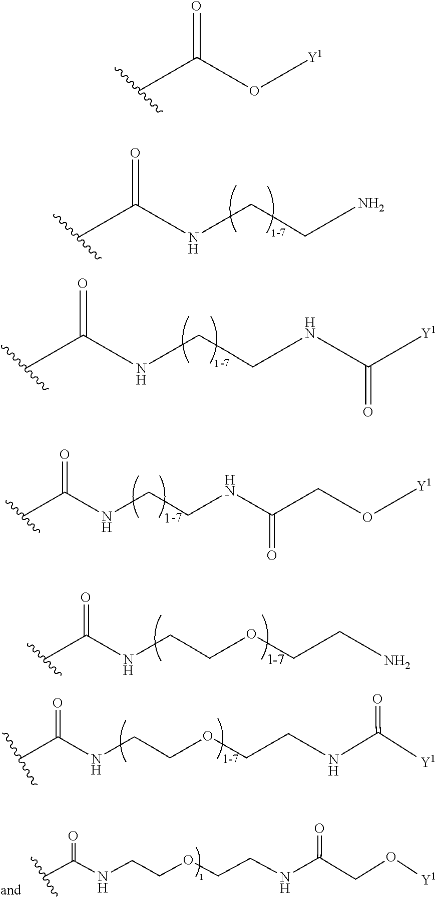

groups.

In some embodiments, each R² is identical and chosen from

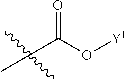

groups.

In some embodiments, each R² is

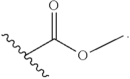 [SMILES: *C(=O)OC]

In some embodiments, each R² is identical and chosen from

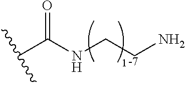

groups.

In some embodiments, each R² is identical and chosen from

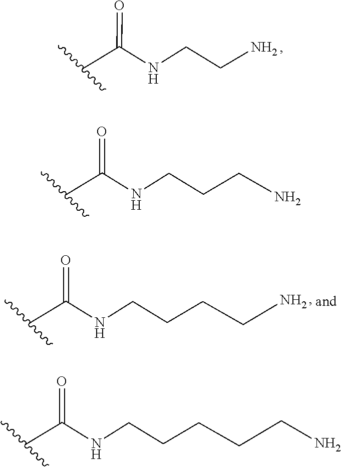 [SMILES: *C(=O)NCCN ; *C(=O)NCCCN ; *C(=O)NCCCCN ; *C(=O)NCCCCCN]

In some embodiments, each R² is identical and chosen from

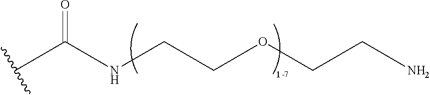

groups.

In some embodiments, each R² is identical and chosen from

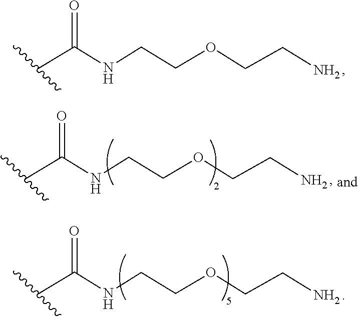

In some embodiments, each R² is identical and chosen from

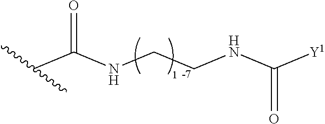

groups.

In some embodiments each R² is

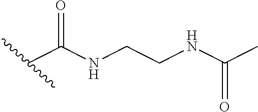 [SMILES: *C(=O)NCCNC(C)=O]

In some embodiments, each R² is identical and chosen from

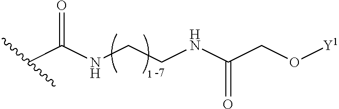

groups.

In some embodiments, each R² is

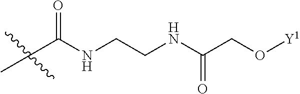

In some embodiments, each Y¹ is identical and chosen from C₁₋₄ alkyl groups.

In some embodiments, each Y¹ is methyl.

In some embodiments, at least one R² is chosen from a linker-non-glycomimetic moiety, wherein the non-glycomimetic moiety is chosen from galectin-3 inhibitors. In some embodiments, at least one galectin-3 inhibitor is chosen from

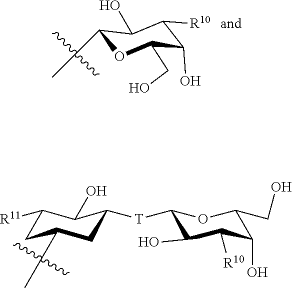

groups, wherein each T, which may be identical or different, is independently chosen from —O— and —S—, and each R¹⁰ and each R₁₁, which may be identical or different, are independently chosen from C₆₋₁₈ aryl, C₁₋₁₃ heteroaryl, C₇₋₁₉ arylalkyl, C₇₋₁₉ arylalkoxy, C₂₋₁₄ heteroarylalkyl, C₂₋₁₄ heteroarylalkoxy, and —NHC(═O)Y; groups, wherein each Y³, which may be identical or different, is independently chosen from C₁₋₈ alkyl, C₂₋₁₂ heterocyclyl, C₆₋₁₈ aryl, and C₁₋₁₃ heteroaryl groups.

In some embodiments, at least one galectin-3 inhibitor is chosen from

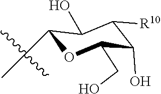

groups.

In some embodiments, at least one galectin-3 inhibitor is chosen from

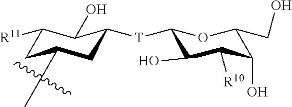

groups.

In some embodiments, at least one T is —O—. In some embodiments, at least one T is —S—.

In some embodiments, each R², which may be identical or different, is independently chosen from a linker-non-glycomimetic moiety, wherein each non-glycomimetic moiety, which may be identical or different, is independently chosen from galectin-3 inhibitors. In some embodiments, each galectin-3 inhibitor, which may be identical or different, is independently chosen from

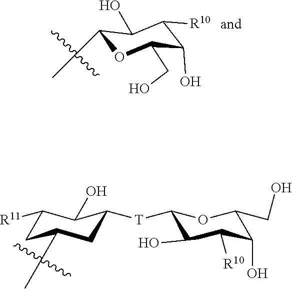

groups.

In some embodiments, each galectin-3 inhibitor, which may be identical or different, is independently chosen from

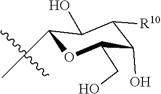

groups.

In some embodiments, each galectin-3 inhibitor, which may be identical or different, is independently chosen from

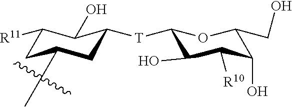

groups.

In some embodiments, each R² is identical and chosen from a linker-non-glycomimetic moiety, wherein the non-glycomimetic moiety is chosen from galectin-3 inhibitors. In some embodiments, each galectin-3 inhibitor is identical and chosen from

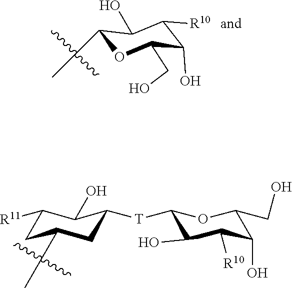

groups.

In some embodiments, each galectin-3 inhibitor is identical and chosen from

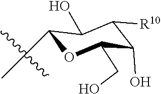

groups.

In some embodiments, each galectin-3 inhibitor is identical and chosen from

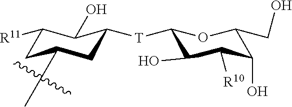

groups.

In some embodiments, each T is —O—. In some embodiments, each T is —S—.

In some embodiments, each R¹⁰ and each R¹¹, which may be identical or different, are independently chosen from C₆₋₁₈ aryl, C₁₋₁₃ heteroaryl, C₇₋₁₉ arylalkoxy, C₂₋₁₄ heteroarylalkyl, C₂₋₁₄ heteroarylalkoxy, and —NHC(═O)Y³ groups, wherein each Y³, which may be identical or different, is independently chosen from C₁₋₈ alkyl and C₆₋₁₈ aryl groups. In some embodiments, each Y³ is chosen from C₁₋₈ alkyl groups. In some embodiments, each Y³ is chosen from C₆₋₁₈ aryl groups.

In some embodiments, each R¹⁰ and each R¹¹, which may be identical or different, are independently chosen from C₆₋₁₈ aryl groups. In some embodiments, each R¹⁰ and each R¹¹, which may be identical or different, are independently chosen from C₁₋₁₃ heteroaryl groups. In some embodiments, each R¹⁰ and each R¹¹, which may be identical or different, are independently chosen from C₇₋₁₉ arylalkoxy groups. In some embodiments, each R¹⁰ and each R¹¹, which may be identical or different, are independently chosen from C₂₋₁₄ heteroarylalkyl groups. In some embodiments, each R¹⁰ and each R¹¹, which may be identical or different, are independently chosen from C₂₋₁₄ heteroarylalkoxy groups. In some embodiments, each R¹⁰ and each R¹¹, which may be identical or different, are independently chosen from —NHC(═O)Y³ groups, wherein each Y³, which may be identical or different, is independently chosen from C₁₋₈ alkyl and C₆₋₁₈ aryl groups.

In some embodiments, at least one R¹¹ or at least one R¹¹ is chosen from

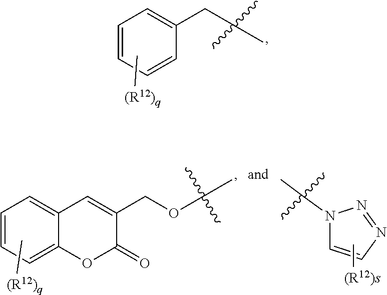

groups, wherein each p, which may be identical or different, is independently chosen from integers ranging from 0 to 5, each q, which may be identical or different, is independently chosen from integers ranging from 0 to 4, each s, which may be identical or different, is independently chosen from integers ranging from 0 to 2, and wherein each R², which may be identical or different, is independently chosen from H, halo, —OH, —OY⁴, —OC(═O)Y⁴, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups, wherein each Y⁴, which may be identical or different, is independently chosen from C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₆₋₁₈ aryl, and C₁₋₁₃ heteroaryl groups.

In some embodiments, at least one R¹⁰ or at least one R¹¹ is chosen from

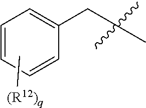

groups.

In some embodiments, at least one R¹⁰ or at least one R¹¹ is chosen from

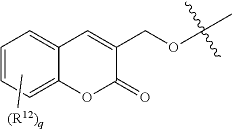

groups.

In some embodiments, at least one R¹⁰ or at least one R¹¹ is chosen from

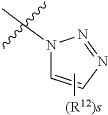

groups.

In some embodiments, at least one R¹⁰ and at least one R¹¹ is independently chosen from

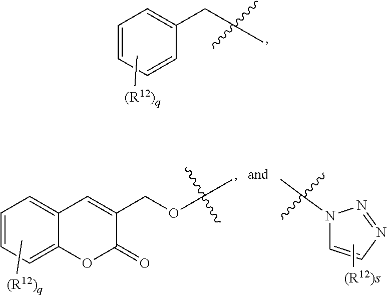

groups.

In some embodiments, at least one R¹⁰ and at least one R¹¹ is independently chosen from

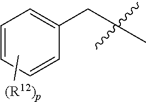

groups.

In some embodiments, at least one R¹⁰ and at least one R¹¹ is independently chosen from

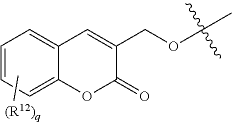

groups.

In some embodiments, at least one R¹⁰ and at least one R¹¹ is independently chosen from

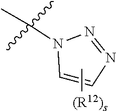

groups.

In some embodiments, each R¹⁰ or each R¹¹ is independently chosen from

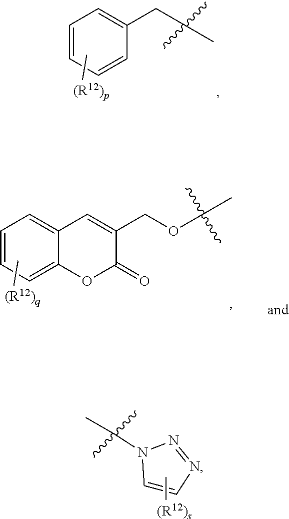

groups.

In some embodiments, each R¹⁰ or each R¹¹ is independently chosen from

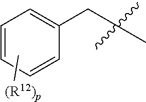

groups.

In some embodiments each R¹⁰ or each R¹¹ is independently chosen from

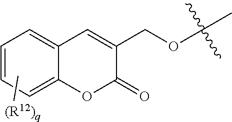

groups.

In some embodiments, each R¹⁰ or each R¹¹ is independently chosen from

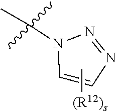

groups.

In some embodiments, each R¹⁰ is identical or each R¹¹ is identical and chosen from

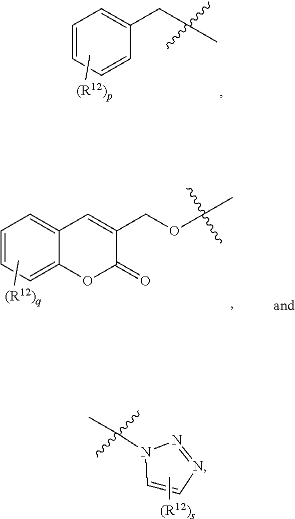

groups.

In some embodiments, each R¹⁰ is identical or each R¹¹ is identical and chosen from

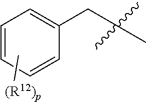

groups.

In some embodiments, each R¹⁰ is identical or each R¹¹ is identical and chosen from

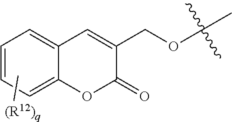

groups.

In some embodiments, each R¹ is identical or each R¹¹ is identical and chosen from

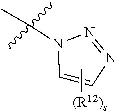

groups.

In some embodiments, at least one R¹⁰ or at least one R¹¹ is

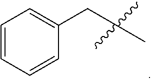 [SMILES: *Cc1ccccc1]

In some embodiments, at least one R¹⁰ or at least one R¹¹ is

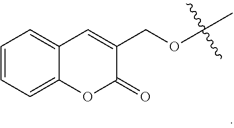 [SMILES: *OCc1cc2ccccc2oc1=O]

In some embodiments, at least one R¹⁰ or at least one R¹¹ is

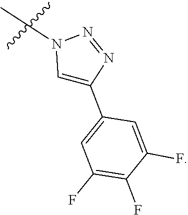 [SMILES: *n1cc(-c2cc(F)c(F)c(F)c2)nn1]

In some embodiments, at least one R¹⁰ or at least one R¹¹ is

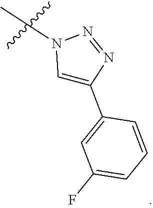 [SMILES: *n1cc(-c2cccc(F)c2)nn1]

In some embodiments, each R¹⁰ or each R¹¹ is

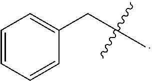 [SMILES: *Cc1ccccc1]

In some embodiments, each R¹⁰ or each R¹¹ is

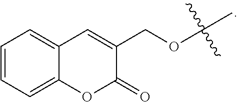 [SMILES: *OCc1cc2ccccc2oc1=O]

In some embodiments, each R¹⁰ or each R¹¹ is

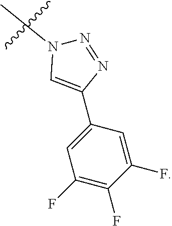 [SMILES: *n1cc(-c2cc(F)c(F)c(F)c2)nn1]

In some embodiments, each R¹⁰ and R¹¹, are

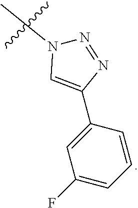 [SMILES: *n1cc(-c2cccc(F)c2)nn1]

In some embodiments, each R¹⁰ and each R¹¹ are

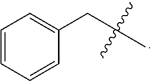 [SMILES: *Cc1ccccc1]

In some embodiments, each R¹⁰ and each R¹¹ are

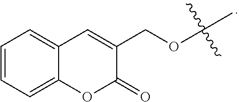 [SMILES: *OCc1cc2ccccc2oc1=O]

In some embodiments, each R¹⁰ and each R¹¹, are

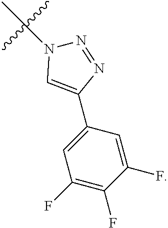 [SMILES: *n1cc(-c2cc(F)c(F)c(F)c2)nn1]

In some embodiments, each R¹⁰ and R¹¹, are

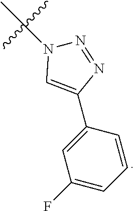 [SMILES: *n1cc(-c2cccc(F)c2)nn1]

In some embodiments at least one galectin-3 inhibitor is

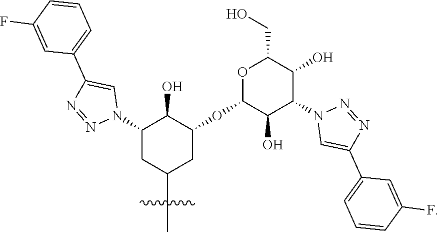 [SMILES: *C1C[C@@H](O[C@@H]2O[C@H](CO)[C@H](O)[C@H](n3cc(-c4cccc(F)c4)nn3)[C@H]2O)[C@H](O)[C@@H](n2cc(-c3cccc(F)c3)nn2)C1]

In some embodiments, each galectin-3 inhibitor is

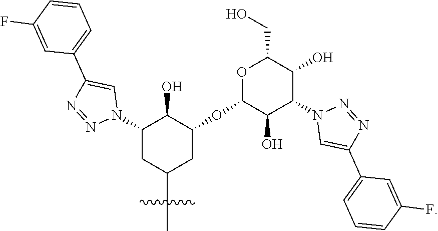 [SMILES: *C1C[C@@H](O[C@@H]2O[C@H](CO)[C@H](O)[C@H](n3cc(-c4cccc(F)c4)nn3)[C@H]2O)[C@H](O)[C@@H](n2cc(-c3cccc(F)c3)nn2)C1]

In some embodiments, at least one galectin-3 inhibitor is chosen from

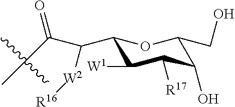

groups,
wherein

each W¹, which may be identical or different, is independently chosen from —O—, —S—, —C—, and —N(R¹⁵)—, wherein each R¹⁵, which may be identical or different, is independently chosen from H, C₁₋₈ alkyl, C₂₋₉ alkenyl, C₂₋₉ alkynyl, C₁₋₈ haloalkyl, C₂₋₈ haloalkenyl, and C₂₋₈ haloalkynyl groups;
each W², which may be identical or different, is independently chosen from H, halo, and —OZ³ groups, wherein each Z³, which may be identical or different, is independently chosen from H and C₁₋₈ alkyl groups;
each R¹⁶, which may be identical or different, is independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, C₆₋₁₈ aryl, C₁₋₁₃ heteroaryl, C₇₋₁₉ arylalkyl, and C₂₋₁₄ heteroarylalkyl groups, wherein the C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, C₆₋₁₈ aryl, C₁₋₁₃ heteroaryl, C₇₋₁₉ arylalkyl, and C₂₋₁₄ heteroarylalkyl groups are optionally substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, —C(═O)NZ⁴Z⁵, and —SO₂Z⁴ groups, wherein each of Z⁴ and V, which may be identical or different, are independently chosen from H, C₁₋₈ alkyl, and C₁₋₈ haloalkyl groups, or Z⁴ and Z⁵ join together along with the nitrogen atom to which they are attached to form a ring;

each R¹⁷, which may be identical or different, is independently chosen from C₆₋₁₈ aryl and C₁₋₁₃ heteroaryl groups, wherein the C₆₋₁₈ aryl and C₁₋₁₃ heteroaryl groups are optionally substituted with one or more groups independently chosen from R¹⁸, C₁₋₈ alkyl, C₁₋₈ haloalkyl, —C(═O)OZ⁶, and —C(═O)NZ⁶Z⁷ groups, wherein each R¹⁸, which may be identical or different, is independently chosen from C₆₋₁₈ aryl groups optionally substituted with one or more groups independently chosen from halo, C₁₋₈ alkyl, —OZ⁸, —C(═O)OZ⁸, and —C(═O)NZ⁸Z⁹ groups, wherein each Z⁶, each Z⁷, each Z⁸ and each Z⁹, which may be identical or different, are independently chosen from H and C₁₋₈ alkyl groups, or Z⁶ and Z⁷ join together along with the nitrogen atom to which they are attached to form a ring and/or Z⁸ and Z⁹ join together along with the nitrogen atom to which they are attached to form a ring; and

wherein each of Z³, Z⁴, Z⁵, Z⁶, Z⁷, Z⁸, and Z⁹ is optionally substituted with one or more groups independently chosen from halo and —OR¹⁹ groups, wherein R¹⁹ is independently chosen from H and Cis alkyl groups.

In some embodiments, at least one galectin-3 inhibitor is chosen from

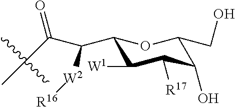

groups.

In some embodiments, at least one galectin-3 inhibitor is chosen from

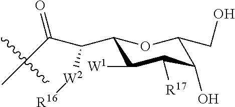

groups.

In some embodiments, each galectin-3 inhibitor, which may be identical or different, is independently chosen from

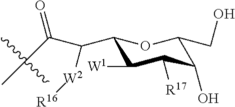

groups.

In some embodiments, each galectin-3 inhibitor, which may be identical or different, is independently chosen from

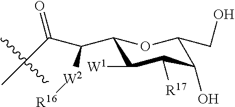

groups.

In some embodiments, each galectin-3 inhibitor, which may be identical or different, is independently chosen from

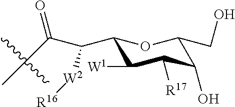

groups.

In some embodiments, each galectin-3 inhibitor is identical and chosen from

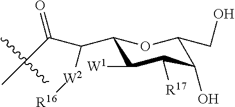

groups.

In some embodiments, each galectin-3 inhibitor is identical and chosen from

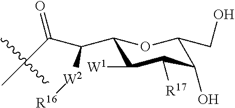

groups.

In some embodiments, each galectin-3 inhibitor is identical and chosen from

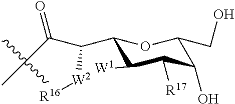

groups.

In some embodiments, each W², which may be identical or different, is independently chosen from —C—, —O—, —S—, and —N(R¹⁵)—, wherein each R¹⁵, which may be identical or different, is independently chosen from H, C₁₋₈ alkyl, and C₁₋₈ haloalkyl groups. In some embodiments, each W² is identical and chosen from —C—, —O—, —S—, and —N(R¹⁵)—, wherein each R¹⁵ is chosen from H, C₁₋₈ alkyl, and C₁₋₈ haloalkyl groups. In some embodiments, each W² is —C—. In some embodiments, each W² is —O—. In some embodiments, each W² is —S—. In some embodiments, each W² is —N(R¹⁵)—. In some embodiments, each R¹⁵, which may be identical or different, is independently chosen from H, C₁₋₄ alkyl, and C₁₋₄ haloalkyl groups. In some embodiments, each R¹⁵ is identical and chosen from H, C₁₋₄ alkyl, and C₁₋₄ haloalkyl groups. In some embodiments, each R¹⁵ is H. In some embodiments, each R¹⁵, which may be identical or different, is independently chosen from C₁₋₄ alkyl groups. In some embodiments, each R¹⁵ is identical and chosen from C₁₋₄ alkyl groups.

In some embodiments, each W¹ is H. In some embodiments, each W¹, which may be identical or different, is independently chosen from halo groups. In some embodiments, each W¹ is identical and chosen from halo groups. In some embodiments, each W¹ is fluoro. In some embodiments, each W¹, which may be identical or different, is independently chosen from —OZ³ groups. In some embodiments, each W¹ is identical and chosen from —OZ³ groups. In some embodiments, each Z³, which may be identical or different, is independently chosen from H and C₁₋₄ alkyl groups. In some embodiments, each Z³ is identical and chosen from H and C₁₋₄ alkyl groups. In some embodiments, each W¹ is —OH. In some embodiments, each W¹ is —OMe.

In some embodiments, each R¹⁶, which may be identical or different, is independently chosen from H, C₁₋₈ alkyl, C₄₋₁₆ cycloalkylalkyl, C₇₋₁₉ arylalkyl, and C₂₋₁₄ heteroarylalkyl groups, wherein the C₁₋₈ alkyl, C₄₋₁₆ cycloalkylalkyl, C₇₋₁₉ arylalkyl, and C₂₋₁₄ heteroarylalkyl groups are optionally substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, —C(═O)NZ⁴Z⁵, and —SO₂Z⁴ groups, wherein each of Z⁴ and Z⁵, which may be identical or different, are independently chosen from H, C₁₋₈ alkyl, and C₁₋₈ haloalkyl groups, or Z⁴ and Z⁵ join together along with the nitrogen atom to which they are attached to form a ring. In some embodiments, each R¹⁶ is identical and chosen from H, C₁₋₈ alkyl, C₄₋₁₆ cycloalkylalkyl, C₇₋₁₉ arylalkyl, and C₂₋₁₄ heteroarylalkyl groups, wherein the C₁₋₈ alkyl, C₄₋₁₆ cycloalkylalkyl, C₇₋₁₉ arylalkyl, and C₂₋₁₄ heteroarylalkyl groups are optionally substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, —C(═O)NZ⁴Z⁵, and —SO₂Z⁴ groups.

In some embodiments, each R¹⁶, which may be identical or different, is independently chosen from H, C₁₋₈ alkyl, and C₄₋₁₆ cycloalkylalkyl groups. In some embodiments, each R¹⁶ is identical and chosen from H, C₁₋₈ alkyl, and C₄₋₁₆ cycloalkylalkyl groups. In some embodiments, each R¹⁶ is identical and chosen from H, C₁₋₄ alkyl, and C₄₋₈ cycloalkylalkyl groups. In some embodiments, each R¹⁶ is H. In some embodiments, each R¹⁶ is identical and chosen from C₁₋₈ alkyl groups. In some embodiments, each R¹⁶ is identical and chosen from C₁₋₄ alkyl groups. In some embodiments, each R¹⁶ is identical and chosen from methyl, ethyl, propyl, and butyl groups. In some embodiments, each R¹⁶ is methyl. In some embodiments, each R¹⁶ is identical and chosen from C₄₋₁₆ cycloalkylalkyl groups. In some embodiments, each R¹⁶ is identical and chosen from C₄₋₈ cycloalkylalkyl groups. In some embodiments, each R¹⁶ is identical and chosen from cyclohexylmethyl and cyclopropylmethyl. In some embodiments, each R¹⁶ is cyclopropylmethyl.

In some embodiments, each R¹⁶, which may be identical or different, is independently chosen from C₇₋₁₉ arylalkyl and C₂₋₁₄ heteroarylalkyl groups, wherein the C₇₋₁₉ arylalkyl and C₂₋₁₄ heteroarylalkyl groups are unsubstituted. In some embodiments, each R¹⁶ is identical and chosen from C₇₋₁₉ arylalkyl and C₂₋₁₄ heteroarylalkyl groups, wherein the C₇₋₁₉ arylalkyl and C₂₋₁₄ heteroarylalkyl groups are unsubstituted. In some embodiments, each R¹⁶, which may be identical or different, is independently chosen from C₇₋₁₉ arylalkyl and C₂₋₁₄ heteroarylalkyl groups, wherein the C₇₋₁₉ arylalkyl and C₂₋₁₄ heteroarylalkyl groups are substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, and —SO₂Z⁴ groups. In some embodiments, each R¹⁶ is identical and chosen from C₇₋₁₉ arylalkyl and C₂₋₁₄ heteroarylalkyl groups, wherein the C₇₋₁₉ arylalkyl and C₂₋₁₄ heteroarylalkyl groups are substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, and —SO₂Z⁴ groups.

In some embodiments, each R¹⁶, which may be identical or different, is independently chosen from C₇₋₁₉ arylalkyl groups. In some embodiments, each R¹⁶ is identical and chosen from C₇₋₁₉ arylalkyl groups. In some embodiments, each R¹⁶ is identical and chosen from C₇₋₁₅ arylalkyl groups. In some embodiments, each R¹⁶ is identical and chosen from C₇₋₁₁ arylalkyl groups. In some embodiments, each R¹⁶, which may be identical or different, is independently chosen from C₂₋₁₄ heteroarylalkyl groups. In some embodiments, each R¹⁶ is identical and chosen from C₂₋₁₄ heteroarylalkyl groups. In some embodiments, each R¹⁶ is identical and chosen from C₄₋₁₄ heteroarylalkyl groups. In some embodiments, each R¹⁶ is identical and chosen from C₂₋₁₀ heteroarylalkyl groups. In some embodiments, each R¹⁶ is identical and chosen from C₄₋₁₀ heteroarylalkyl groups.

In some embodiments, each R¹⁶, which may be identical or different, is independently chosen from C₇₋₁₉ arylalkyl groups, wherein the C₇₋₁₉ arylalkyl groups are unsubstituted. In some embodiments, each R¹⁶ is identical and chosen from C₇₋₁₉ arylalkyl groups, wherein the C₇₋₁₉ arylalkyl groups are unsubstituted. In some embodiments, each R¹⁶ is identical and chosen from C₇₋₁₁ arylalkyl groups, wherein the C₇₋₁₁ arylalkyl groups are unsubstituted.

In some embodiments, at least one R¹⁶ is chosen from

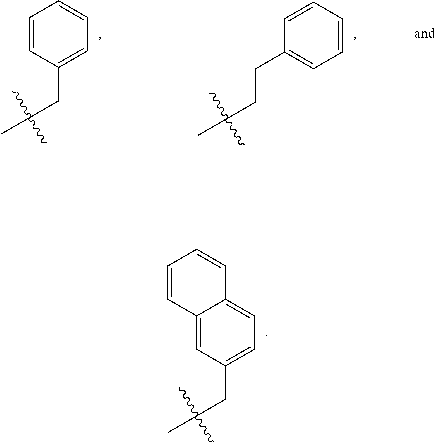 [SMILES: *Cc1ccccc1 ; *CCc1ccccc1 ; *Cc1ccc2ccccc2c1]

In some embodiments, each R¹⁶ is chosen from

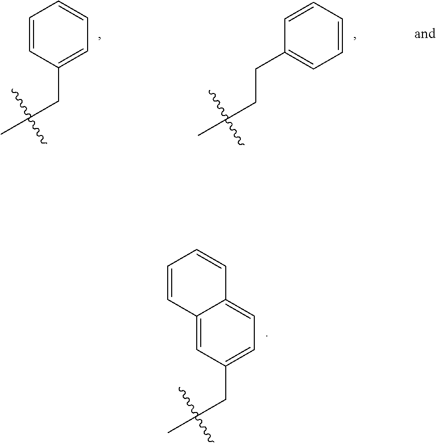 [SMILES: *Cc1ccccc1 ; *CCc1ccccc1 ; *Cc1ccc2ccccc2c1]

In some embodiments, each R¹⁶, which may be identical or different, is independently chosen from C₇₋₁₉ arylalkyl groups, wherein the C₇₋₁₉ arylalkyl groups are substituted with one or more groups independently chosen from halo groups. In some embodiments, each R¹⁶ is identical and chosen from C₇₋₁₉ arylalkyl groups, wherein the C₇₋₁₉ arylalkyl groups are substituted with one or more groups independently chosen from halo groups. In some embodiments, the halo group is independently chosen from fluoro and chloro. In some embodiments, at least one halo group is fluoro. In some embodiments, at least one halo group is chloro.

In some embodiments at least one R¹⁶ is chosen from

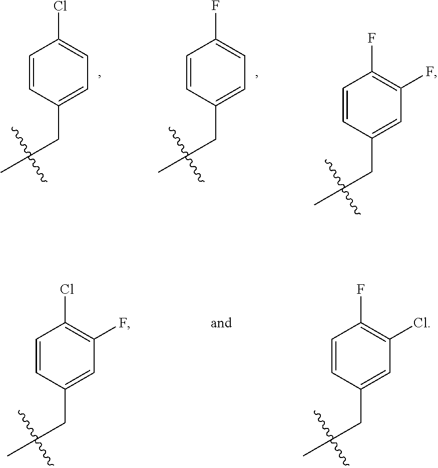 [SMILES: *Cc1ccc(Cl)cc1 ; *Cc1ccc(F)cc1 ; *Cc1ccc(F)c(F)c1 ; *Cc1ccc(Cl)c(F)c1 ; "and" ; *Cc1ccc(F)c(Cl)c1]

In some embodiments, each R¹⁶ is chosen from

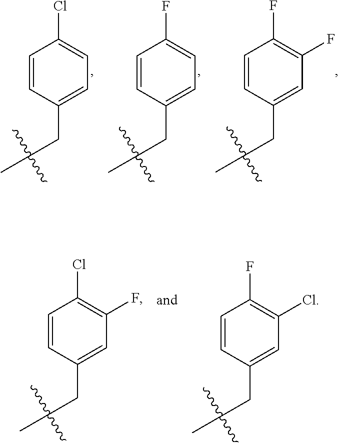 [SMILES: *Cc1ccc(Cl)cc1 ; *Cc1ccc(F)cc1 ; *Cc1ccc(F)c(F)c1 ; *Cc1ccc(Cl)c(F)c1 ; *Cc1ccc(F)c(Cl)c1]

In some embodiments, each R¹⁶, which may be identical or different, is independently chosen from C₇₋₁₉ arylalkyl groups, wherein the C₇₋₁₉ arylalkyl groups are substituted with one or more groups independently chosen from C₁₋₈ alkyl, C₁₋₈ hydroxyalkyl, C₁₋₈ haloalkyl, and C₆₋₁₈ aryl groups. In some embodiments, each R¹⁶ is identical and chosen from C₇₋₁₉ arylalkyl groups, wherein the C₇₋₁₉ arylalkyl groups are substituted with one or more groups independently chosen from C₁₋₈ alkyl, C₁₋₈ hydroxyalkyl, C₁₋₈ haloalkyl, and C₆₋₁₈ is aryl groups. In some embodiments, at least one R¹⁶ is benzyl, wherein the benzyl is substituted with one or more groups independently chosen from C₁₋₈ alkyl, C₁₋₈ hydroxyalkyl, C₁₋₈ haloalkyl, and C₆₋₁₈ aryl groups. In some embodiments, each R¹⁶ is benzyl, wherein the benzyl is substituted with one or more groups independently chosen from C₁₋₈ alkyl, C₁₋₈ hydroxyalkyl, C₁₋₈ haloalkyl, and C₆₋₁₈ aryl groups.

In some embodiments, at least one R¹⁶ is chosen from

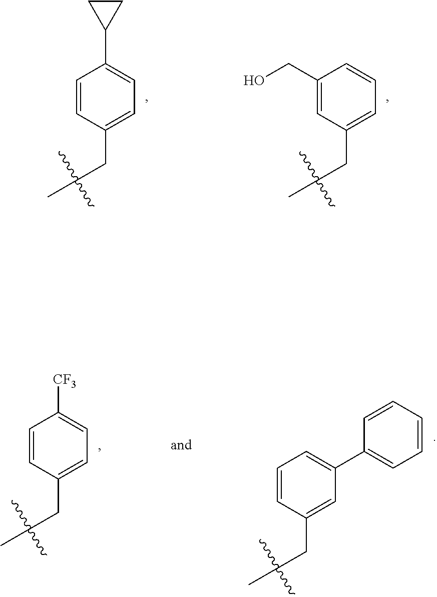 [SMILES: *Cc1ccc(C2CC2)cc1 ; *Cc1cccc(CO)c1 ; *Cc1ccc(C(F)(F)F)cc1 ; *Cc1cccc(-c2ccccc2)c1]

In some embodiments, each R¹⁶ is chosen from

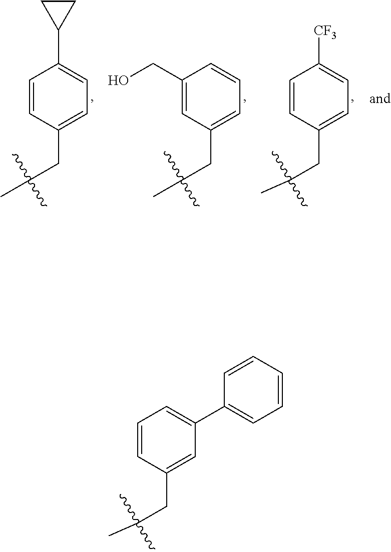

In some embodiments, each R¹⁶, which may be identical or different, is independently chosen from C₇₋₁₉ arylalkyl groups, wherein the C₇₋₁₉ arylalkyl groups are substituted with one or more groups independently chosen from —OZ⁴, —C(═O)OZ⁴, and —SO₂Z⁴ groups, wherein Z⁴ is independently chosen from H, C₁₋₈ alkyl, and C₁₋₈ haloalkyl groups. In some embodiments, each R¹⁶ is identical and chosen from C₇₋₁₉ arylalkyl groups, wherein the C₇₋₁₉ arylalkyl groups are substituted with one or more groups independently chosen from —OZ⁴, —C(═O)OZ⁴, and —SO₂Z⁴ groups, wherein Z⁴ is independently chosen from H, C₁₋₈ alkyl, and C₁₋₈ haloalkyl groups. In some embodiments, at least one R¹⁶ is benzyl, wherein the benzyl is substituted with one or more groups independently chosen from —OZ⁴, —C(═O)OZ⁴, and —SO₂Z⁴ groups. In some embodiments, each R¹⁶ is benzyl, wherein the benzyl is substituted with one or more groups independently chosen from —OZ⁴, —C(═O)OZ⁴, and —SO₂Z⁴ groups. In some embodiments, each Z⁴, which may be identical or different, is independently chosen from H, C₁₋₄ alkyl, and C₁₋₄ haloalkyl groups. In some embodiments, each Z⁴ is identical and chosen from H, C₁₋₄ alkyl, and C₁₋₄ haloalkyl groups. In some embodiments, each Z⁴ is H. In some embodiments, each Z⁴ is identical and chosen from C₁₋₄ alkyl groups. In some embodiments, each Z⁴ is methyl. In some embodiments, each Z⁴ is identical and chosen from C₁₋₄ haloalkyl groups. In some embodiments, each Z⁴ is —CF₃.

In some embodiments, at least one R¹⁶ is chosen from

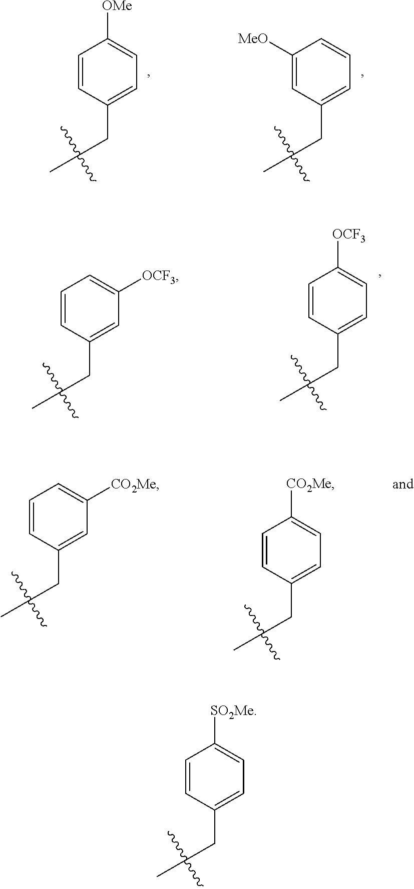

In some embodiments, each R¹⁶ is chosen from

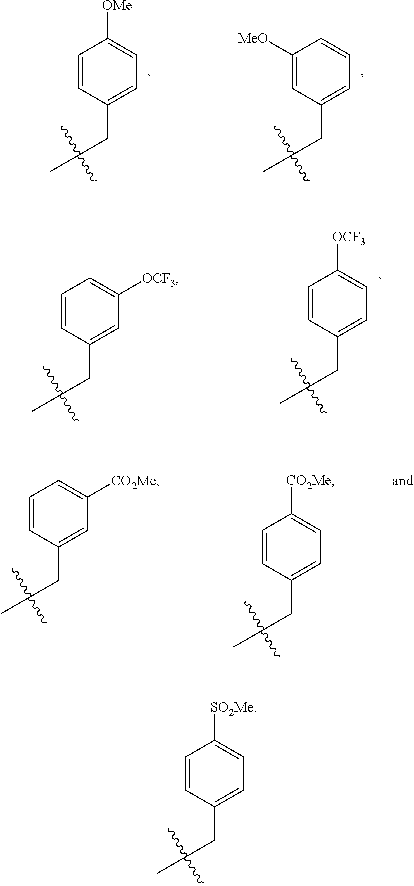

In some embodiments, each R¹⁶, which may be identical or different, is independently chosen from C₂₋₁₄ heteroarylalkyl groups, wherein the C₂₋₁₄ heteroarylalkyl groups are unsubstituted. In some embodiments, each R¹⁶ is identical and chosen from C₂₋₁₄ heteroarylalkyl groups, wherein the C₂₋₁₄ heteroarylalkyl groups are unsubstituted. In some embodiments, each R¹⁶ is identical and chosen from C₂₋₁₀ heteroarylalkyl groups, wherein the C₂₋₁₀ heteroarylalkyl groups are unsubstituted. In some embodiments, each R¹⁶ is identical and chosen from C₄₋₁₄ heteroarylalkyl groups, wherein the C₄₋₁₄ heteroarylalkyl groups are unsubstituted. In some embodiments, each R¹⁶ is identical and chosen from C₄₋₁₀ heteroarylalkyl groups, wherein the C₄₋₁₀ heteroarylalkyl groups are unsubstituted.

In some embodiments, each R¹⁶, which may be identical or different, is independently chosen from C₂₋₁₄ heteroarylalkyl groups, wherein the C₂₋₁₄ heteroarylalkyl groups are optionally substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, and —SO₂Z⁴ groups, wherein Z⁴ is independently chosen from H and C₁₋₈ alkyl groups. In some embodiments, each R¹⁶ is identical and chosen from C₂₋₁₄ heteroarylalkyl groups, wherein the C₂₋₁₄ heteroarylalkyl groups are optionally substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, and —SO₂Z⁴ groups, wherein Z⁴ is independently chosen from H and C₁₋₈ alkyl groups. In some embodiments, each Z⁴, which may be identical or different, is independently chosen from H and methyl. In some embodiments, each Z⁴ is identical and chosen from H and methyl. In some embodiments, each Z⁴ is H. In some embodiments, each Z⁴ is methyl.

In some embodiments, at least one R¹⁶ is chosen from

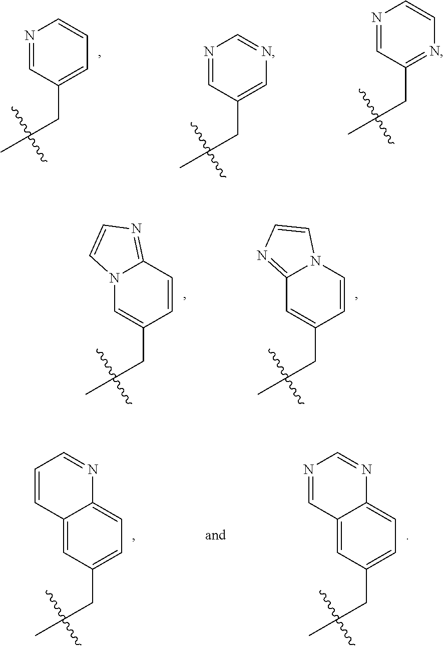

In some embodiments, each R¹⁶ is chosen from

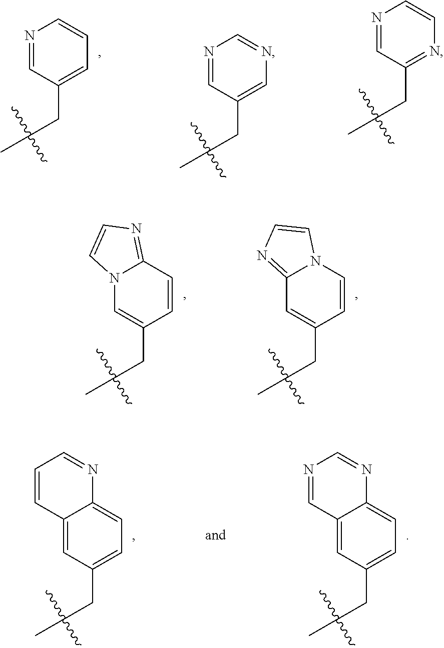

In some embodiments, each R¹⁷, which may be identical or different, is independently chosen from C₁₋₁₃ heteroaryl groups optionally substituted with one or more groups independently chosen from R¹⁸, C₁₋₈ alkyl, C₁₋₈ haloalkyl, —C(═O)OZ⁶, and —C(═O)NZ⁶Z⁷ groups. In some embodiments, each R⁷ is identical and chosen from C₁₋₁₃ heteroaryl groups optionally substituted with one or more groups independently chosen from R¹⁸, C₁₋₈ alkyl, C₁₋₈ haloalkyl, —C(═OZ⁶, and —C(═O)NZ⁶Z⁷ groups. In some embodiments, each R¹⁷, which may be identical or different, is independently chosen from C₁₋₁₃ heteroaryl groups substituted with one or more groups independently chosen from R¹⁸, C₁₋₈ alkyl, C₁₋₈ haloalkyl, —C(═OZ⁶, and —C(═O)NZ⁶Z⁷ groups. In some embodiments, each R¹⁷ is identical and chosen from C₁₋₁₃ heteroaryl groups substituted with one or more groups independently chosen from R¹⁸, C₁₋₈ alkyl, C₁₋₈ haloalkyl, —C(═OZ⁶, and —C(═O)NZ⁶Z⁷ groups. In some embodiments, each R⁷ is identical and chosen from C₂₋₆ heteroaryl groups. In some embodiments, each R¹⁷ is identical and chosen from C₂₋₄ heteroaryl groups.

In some embodiments, each R¹⁷, which may be identical or different, is independently chosen from C₁₋₁₃ heteroaryl groups optionally substituted with one or more groups independently chosen from R¹⁸. In some embodiments, each R¹⁷ is identical and chosen from C₁₋₁₃ heteroaryl groups optionally substituted with one or more groups independently chosen from R¹⁸. In some embodiments, each R¹⁷ is identical and chosen from C₂₋₄ heteroaryl groups optionally substituted with one or more groups independently chosen from R¹⁸. In some embodiments, each R¹⁷, which may be identical or different, is independently chosen from C₁₋₁₃ heteroaryl groups substituted with one or more groups independently chosen from R¹⁸. In some embodiments, each R¹⁷ is identical and chosen from C₁₋₁₃ heteroaryl groups substituted with one or more groups independently chosen from R¹⁸. In some embodiments, each R¹⁷ is identical and chosen from C₂₋₄ heteroaryl groups substituted with one or more groups independently chosen from R¹⁸.

In some embodiments, each R¹⁸, which may be identical or different, is independently chosen from C₆₋₁₈ aryl groups optionally substituted with one or more groups independently chosen from halo groups. In some embodiments, each R¹⁸ is identical and chosen from C₆₋₁₈ aryl groups optionally substituted with one or more groups independently chosen from halo groups. In some embodiments, each R¹⁸, which may be identical or different, is independently chosen from phenyl optionally substituted with one or more groups independently chosen from halo groups. In some embodiments, each R¹⁸ is identical and chosen from phenyl optionally substituted with one or more groups independently chosen from halo groups. In some embodiments, each R¹⁸, which may be identical or different, is independently chosen from C₆₋₁₈ aryl groups substituted with one or more groups independently chosen from halo groups. In some embodiments, each R¹⁸ is identical and chosen from C₆₋₁₈ aryl groups substituted with one or more groups independently chosen from halo groups. In some embodiments, each R¹⁸, which may be identical or different, is independently chosen from phenyl substituted with one or more groups independently chosen from halo groups. In some embodiments, each R¹⁸ is identical and chosen from phenyl substituted with one or more groups independently chosen from halo groups. In some embodiments, at least one halo group is fluoro.

In some embodiments, at least one R¹⁷ is chosen from

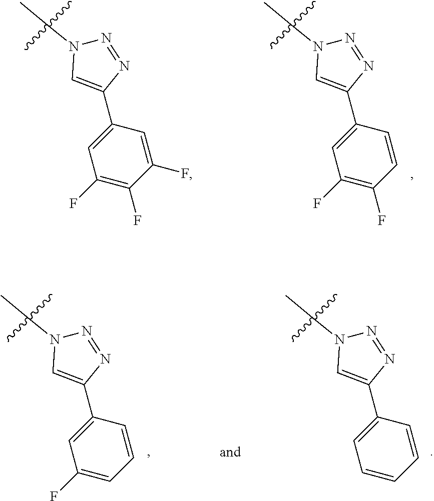

In some embodiments, each R¹⁷ is chosen from

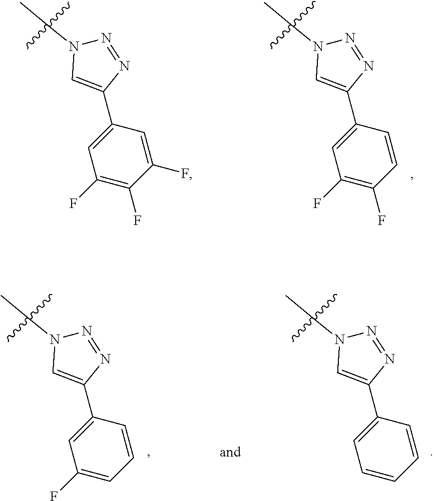

In some embodiments, at least one R¹⁷ is

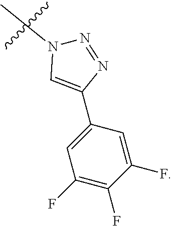 [SMILES: *n1cc(-c2cc(F)c(F)c(F)c2)nn1]

In some embodiments, each R¹⁷ is

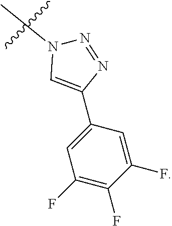 [SMILES: *n1cc(-c2cc(F)c(F)c(F)c2)nn1]

In some embodiments, at least one R¹⁷ is

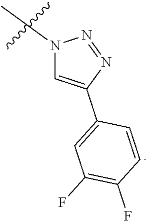 [SMILES: *n1cc(-c2ccc(F)c(F)c2)nn1]

In some embodiments, each R¹⁷ is

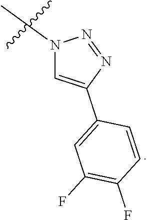 [SMILES: *n1cc(-c2ccc(F)c(F)c2)nn1]

In some embodiments, at least one R¹⁷ is

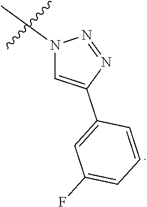 [SMILES: *n1cc(-c2cccc(F)c2)nn1]

In some embodiments, each R¹⁷ is

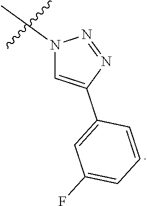 [SMILES: *n1cc(-c2cccc(F)c2)nn1]

In some embodiments, at least one R¹⁷ is

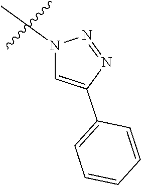 [SMILES: *n1cc(-c2ccccc2)nn1]

In some embodiments, each R¹⁷ is

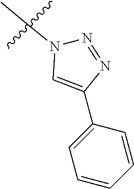 [SMILES: *n1cc(-c2ccccc2)nn1]

In some embodiments, each of Z³, Z⁴, Z⁵, Z⁶, Z⁷, Z⁸, and Z⁹ is unsubstituted. In some embodiments, at least one of Z³, Z⁴, Z³, Z⁶, Z⁷, Z⁸, and Z⁹ is substituted. In some embodiments, at least one of Z³, Z⁴, Z³, Z⁶, Z⁷, Z⁸, and Z⁹ is substituted with one or more groups independently chosen from halo and —OR¹⁹ groups. In some embodiments, at least one of Z³, Z⁴, Z⁵, Z⁶, Z⁷, Z⁸, and Z⁹ is substituted with one or more groups independently chosen from halo groups. In some embodiments, at least one of Z³, Z⁴, Z⁵, Z⁶, Z⁷, Z⁸, and Z⁹ is substituted with one or more groups independently chosen from —OR¹⁹ groups. In some embodiments, at least one R¹⁹ is H. In some embodiments, each R¹⁹ is H. In some embodiments, each R¹⁹, which may be identical or different, is independently chosen from C₁₋₈ alkyl groups. In some embodiments, each R¹⁹ is identical and chosen from C₁₋₈ alkyl groups. In some embodiments, each R¹⁹, which may be identical or different, is independently chosen from C₁₋₄ alkyl groups. In some embodiments, each R¹⁹ is identical and chosen from C₁₋₄ alkyl groups. In some embodiments, each R¹⁹, which may be identical or different, is independently chosen from methyl, ethyl, propyl, and butyl groups. In some embodiments, each R¹⁹ is identical and chosen from methyl, ethyl, propyl, and butyl groups. In some embodiments, at least one halo group is fluoro. In some embodiments, each halo group is fluoro.

In some embodiments, at least one galectin-3 inhibitor is chosen from

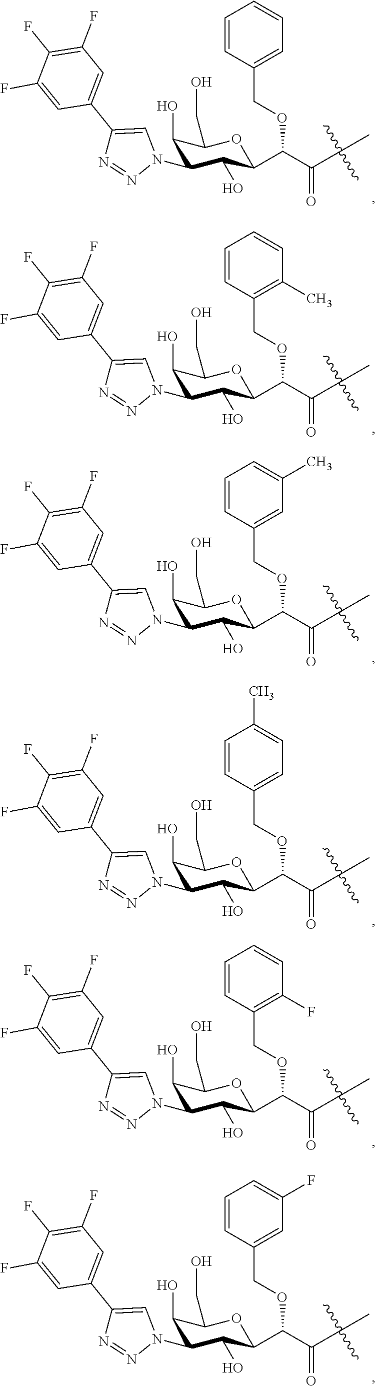

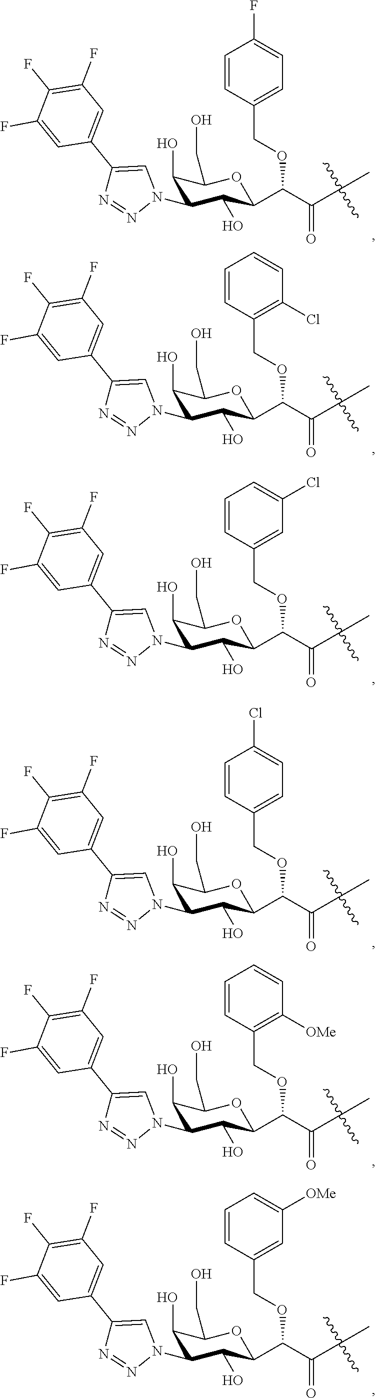

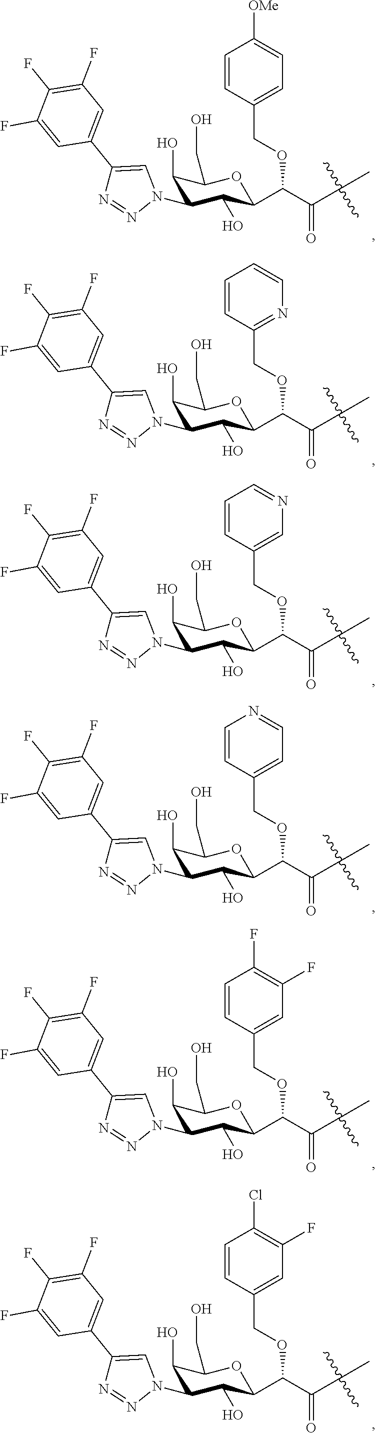

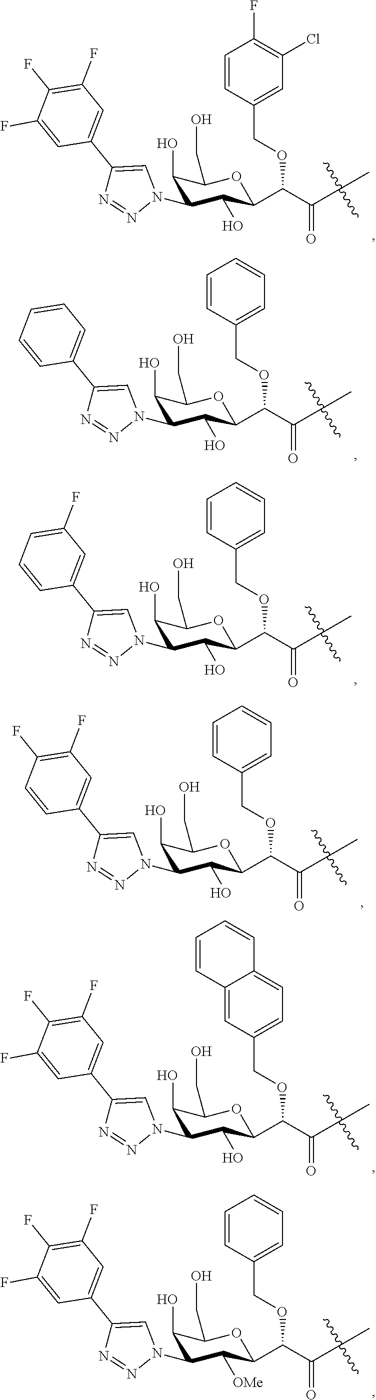

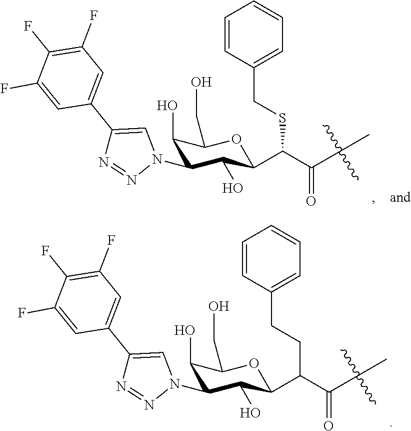

In some embodiments, each galectin-3 inhibitor is chosen from

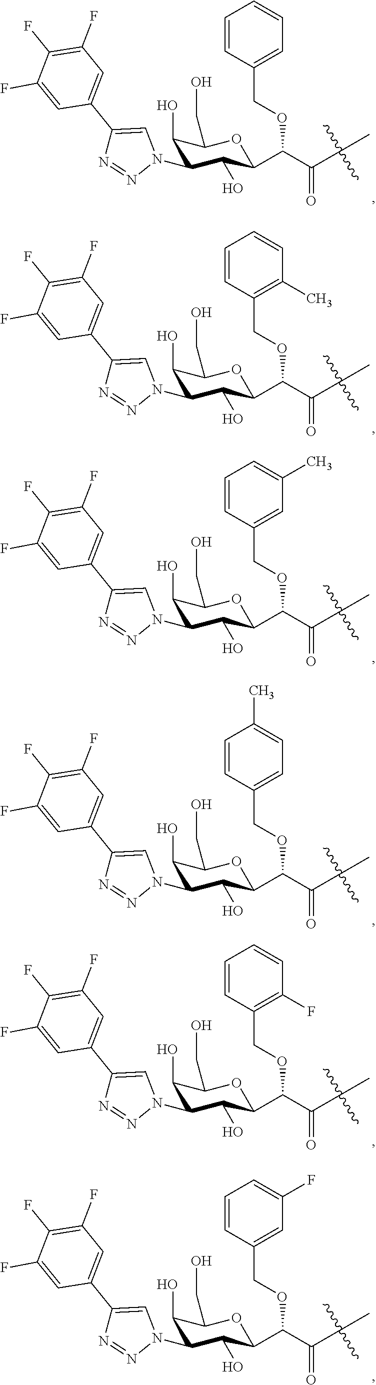

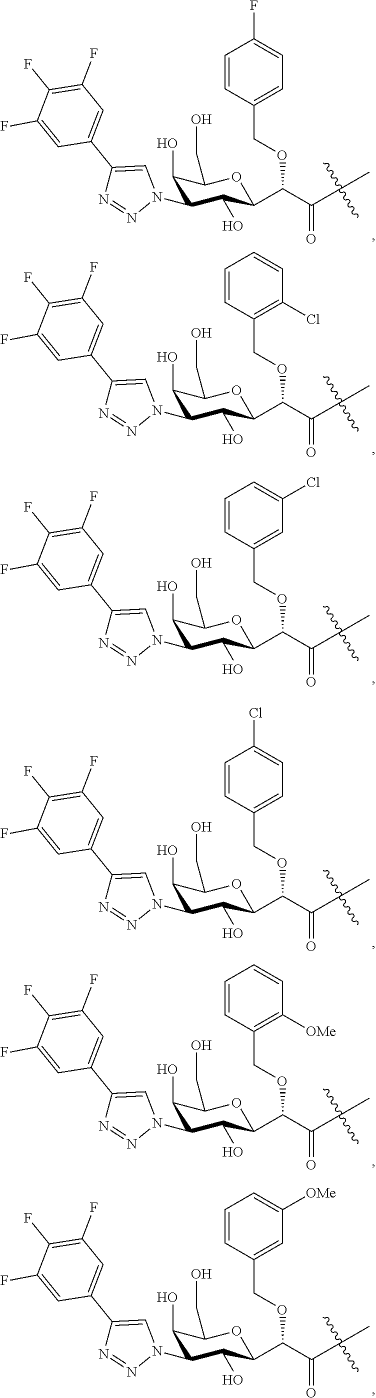

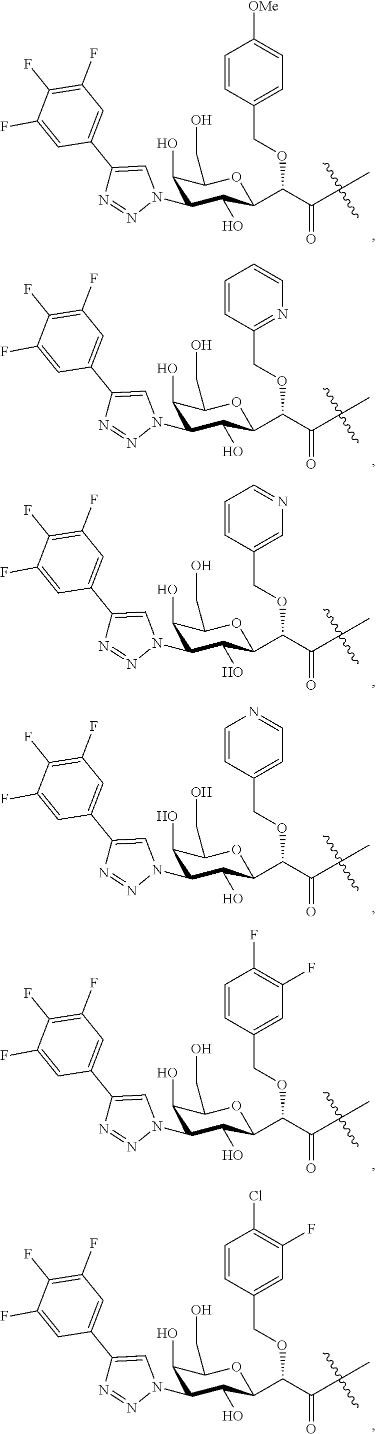

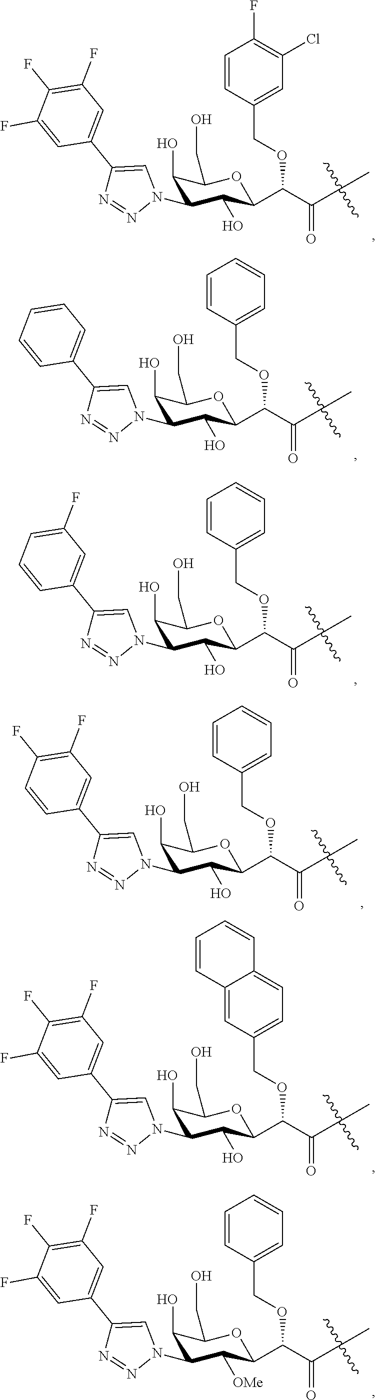

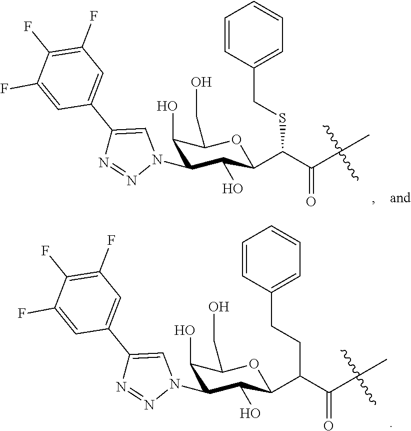

In some embodiments, at least one R² is chosen from a linker-non-glycomimetic moiety, wherein the non-glycomimetic moiety is chosen from CXCR4 chemokine receptor inhibitors. In some embodiments, at least one CXCR4 chemokine receptor inhibitor is chosen from

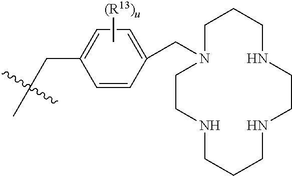

groups, wherein each R¹³, which may be identical or different, is independently chosen from H, halo, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups and wherein u is chosen from integers ranging from 1 to 4.

In some embodiments, at least one R¹³ is independently chosen from H, halo, C₁₋₈ alkyl, and C₁₋₈ haloalkyl groups. In some embodiments, at least one R³ is halo. In some embodiments, at least one R¹³ is fluoro. In some embodiments, at least one R¹³ is chloro. In some embodiments, at least one R¹³ is bromo. In some embodiments, at least one R³ is iodo.

In some embodiments, at least one u is 1. In some embodiments, at least one u is 2. In some embodiments, at least one u is 4.

In some embodiments, at least one CXCR4 chemokine receptor inhibitor is chosen from

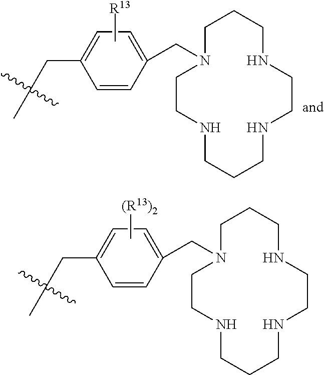

groups, wherein each R¹³, which may be identical or different, is independently chosen from H and halo groups.

In some embodiments, at least one CXCR4 chemokine receptor inhibitor is

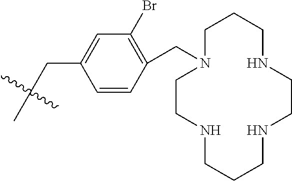 [SMILES: *Cc1ccc(CN2CCCNCCNCCCNCC2)c(Br)c1]

In some embodiments, each R², which may be identical or different, is independently chosen from a linker-non-glycomimetic moiety, wherein each non-glycomimetic moiety, which may be identical or different, is independently chosen from CXCR4 chemokine receptor inhibitors. In some embodiments, each CXCR4 chemokine receptor inhibitor, which may be identical or different, is independently chosen from

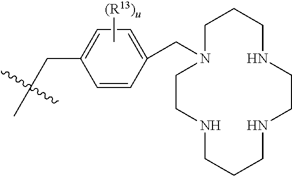

groups, wherein each R³, which may be identical or different, is independently chosen from H, halo, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups and wherein each u, which may be identical or different, is independently chosen from integers ranging from 1 to 4.

In some embodiments, each R¹³, which may be identical or different, is independently chosen from H, halo, C₁₋₈ alkyl, and C₁₋₈ haloalkyl groups. In some embodiments, each R¹³, which may be identical or different, is independently chosen from halo groups.

In some embodiments, each CXCR4 chemokine receptor inhibitor, which may be identical or different, is independently chosen from

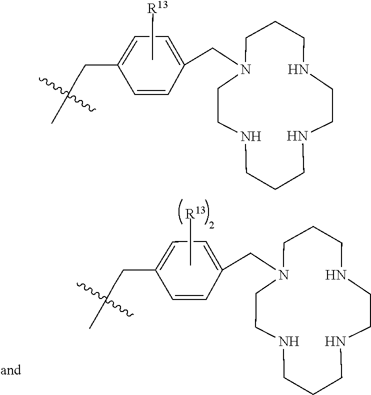

groups, wherein each R¹³, which may be identical or different, is independently chosen from H and halo groups.

In some embodiments, each R² is identical and chosen from a linker-non-glycomimetic moiety, wherein each non-glycomimetic moiety is chosen from CXCR4 chemokine receptor inhibitors. In some embodiments, each CXCR4 chemokine receptor inhibitor is identical and chosen from

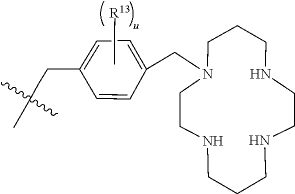

groups, wherein each R¹³, which may be identical or different, is independently chosen from H, halo, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups and wherein each u, which may be identical or different, is independently chosen from integers ranging from 1 to 4.

In some embodiments, each R³ is identical and independently chosen from H, halo, C₁₋₈ alkyl, and C₁₋₈ haloalkyl groups. In some embodiments, each R¹³ is H. In some embodiments, each R¹³ is identical and independently chosen from halo groups. In some embodiments, each R¹³ is fluoro. In some embodiments, each R¹³ is chloro. In some embodiments, each R¹³ is bromo. In some embodiments, each R¹³ is iodo.

In some embodiments, each u is 1. In some embodiments, each u is 2. In some embodiments, each u is 4.

In some embodiments, each CXCR4 chemokine receptor inhibitor is identical and independently chosen from

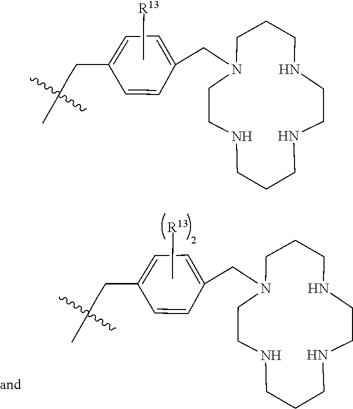

groups, wherein each R³, which may be identical or different, is independently chosen from H and halo groups.

In some embodiments, each CXCR4 chemokine receptor inhibitor is

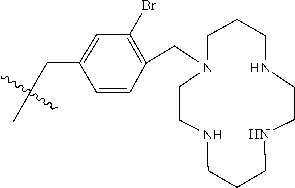 [SMILES: *CCc1ccc(CN2CCCNCCNCCCNCC2)c(Br)c1]

In some embodiments, at least one R² is chosen from a linker-non-glycomimetic moiety, wherein the non-glycomimetic moiety is chosen from R⁸, C₆₋₁₈ aryl-R⁸, and C₁₋₁₂ heteroaryl-R⁸ groups. In some embodiments, at least one R² is chosen from a linker-non-glycomimetic moiety, wherein the non-glycomimetic moiety is chosen from R⁸ groups. In some embodiments, at least one R² is chosen from a linker-non-glycomimetic moiety, wherein the non-glycomimetic moiety is chosen from C₆₋₁₈ aryl-R⁸ groups. In some embodiments, at least one R² is chosen from a linker-non-glycomimetic moiety, wherein the non-glycomimetic moiety is chosen from C₁₋₁₂ heteroaryl-R⁸ groups. In some embodiments, at least one R² is chosen from a linker-non-glycomimetic moiety, wherein the non-glycomimetic moiety is chosen from

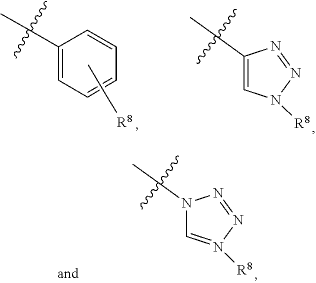

groups.

In some embodiments, at least one R⁸ is chosen from C₁₋₁₂ alkyl groups substituted with at least one substituent chosen from —OH, —OSO₃Q, —OPO₃Q₂, —CO₂Q, and —SO₃Q groups. In some embodiments, at least one R⁸ is chosen from C₂₋₁₂ alkenyl groups substituted with at least one substituent chosen from —OH, —OSO₃Q, —OPO₃Q₂, —CO₂Q, and —SO₃Q groups. In some embodiments, at least one R⁸ is chosen from C₁₋₈ alkyl groups substituted with at least one substituent chosen —OH, —OSO₃Q, —OPO₃Q₂, —CO₂Q, and —SO₃Q groups. In some embodiments, at least one R⁸ is chosen from C₂₋₅ alkenyl groups substituted with at least one substituent chosen from —OH, —OSO₃Q, —OPO₃Q₂, —CO₂Q, and —SO₃Q groups. In some embodiments, at least one R⁸ is chosen from C₁₋₈ alkyl groups substituted with at least one substituent chosen from —OH, —OSO₃Q, —OPO₃Q₂, —CO₂Q, and —SO₃Q groups. In some embodiments, at least one R⁸ is chosen from C₂₋₅ alkenyl groups substituted with at least one substituent chosen from —OH, —OSO₃Q, —OPO₃Q₂, —CO₂Q, and —SO₃Q groups.

In some embodiments, at least one R⁸ is chosen from C₁₋₈ alkyl groups substituted with at least two substituents independently chosen from —OH, —OSO₃Q, —OPO₃Q₂, —CO₂Q, and —SO₃Q groups. In some embodiments, at least one R⁸ is chosen from C₂₋₈ alkenyl groups substituted with at least two substituents independently chosen from —OH, —OSO₃Q, —OPO₃Q₂, —CO₂Q, and —SO₃Q groups. In some embodiments, at least one R⁸ is chosen from C₁₋₅ alkyl groups substituted with at least two substituents independently chosen from —OH, —OSO₃Q, —OPO₃Q₂, —CO₂Q, and —SO₃Q groups. In some embodiments, at least one R⁸ is chosen from C₂₋₅ alkenyl groups substituted with at least two substituents independently chosen from —OH, —OSO₃Q, —OPO₃Q₂, —CO₂Q, and —SO₃Q groups.

In some embodiments, at least one R⁸ is chosen from C₁₋₈ alkyl groups substituted with at least three substituents independently chosen from —OH, —OSO₃Q, —OPO₃Q₂, —CO₂Q, and —SO₃Q groups. In some embodiments, at least one R⁸ is chosen from C₂₋₈ alkenyl groups substituted with at least three substituents independently chosen from —OH, —OSO₃Q, —OPO₃Q₂, —CO₂Q, and —SO₃Q groups. In some embodiments, at least one R⁸ is chosen from C₁₋₅ alkyl groups substituted with at least three substituents independently chosen from —OH, —OSO₃Q, —OPO₃Q₂, —CO₂Q, and —SO₃Q groups. In some embodiments, at least one R⁸ is chosen from C₂₋₅ alkenyl groups substituted with at least three substituents independently chosen from —OH, —OSO₃Q, —OPO₃Q₂, —CO₂Q, and —SO₃Q groups.

In some embodiments, at least one R⁸ is chosen from

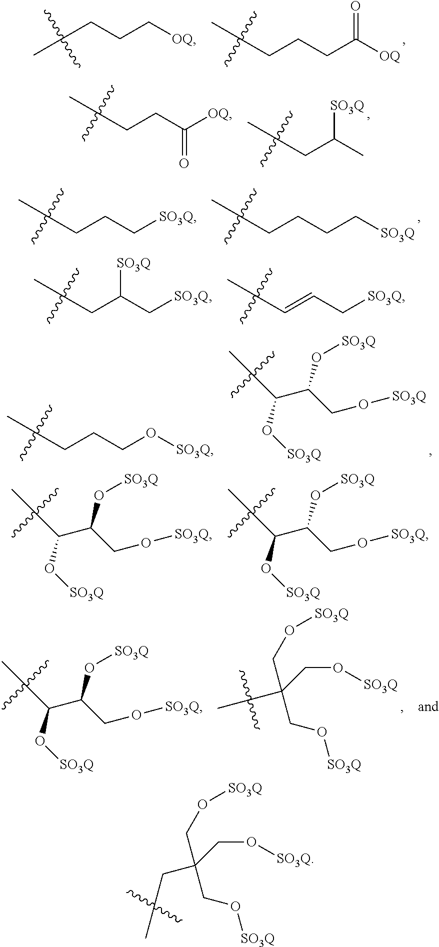

In some embodiments, at least one R⁸ is chosen from

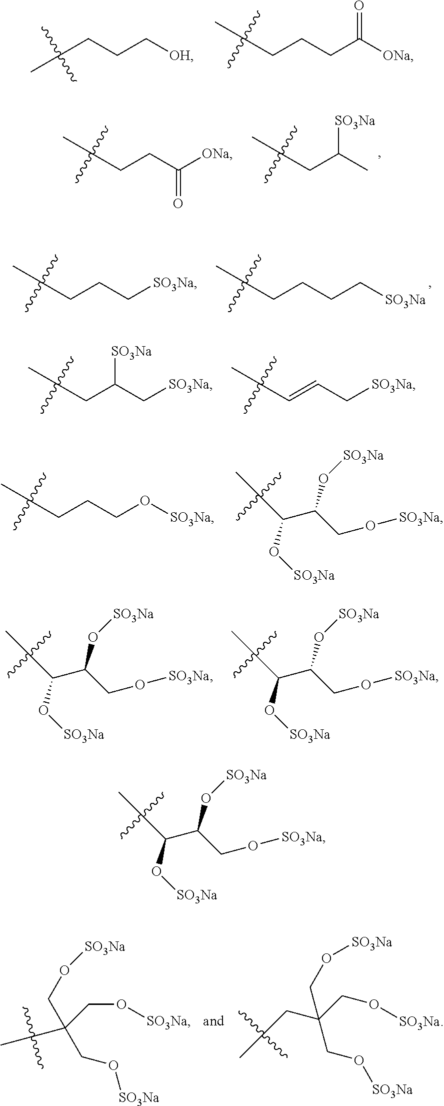

In some embodiments, at least one R² is chosen from a linker-non-glycomimetic moiety, wherein the non-glycomimetic moiety is chosen from

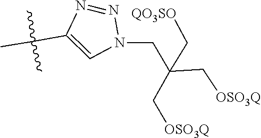

In some embodiments, at least one R² is chosen from a linker-non-glycomimetic moiety, wherein the non-glycomimetic moiety is

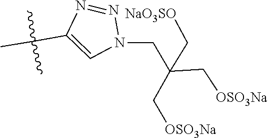

In some embodiments, each R², which may be identical or different, is independently chosen from a linker-non-glycomimetic moiety, wherein each non-glycomimetic moiety, which may be identical or different, is independently chosen from R⁸, C₆₋₁₈ aryl-R⁸, and C₁₋₁₂ heteroaryl-R⁸ groups. In some embodiments, each R², which may be identical or different, is independently chosen from a linker-non-glycomimetic moiety, wherein each non-glycomimetic moiety, which may be identical or different, is independently chosen from Regroups. In some embodiments, each R², which may be identical or different, is independently chosen from a linker-non-glycomimetic moiety, wherein each non-glycomimetic moiety, which may be identical or different, is independently chosen from C₆₋₁₈ aryl-R⁸ groups. In some embodiments, each R², which may be identical or different, is independently chosen from a linker-non-glycomimetic moiety, wherein each non-glycomimetic moiety, which may be identical or different, is independently chosen from C₁₋₁₂ heteroaryl-R⁸ groups. In some embodiments, each R², which may be identical or different, is independently chosen from a linker-non-glycomimetic moiety, wherein each non-glycomimetic moiety, which may be identical or different, is independently chosen from

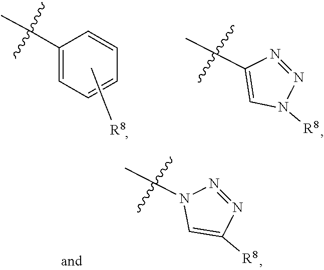

In some embodiments, each R⁸, which may be identical or different, is independently chosen from C₁₋₁₂ alkyl groups substituted with at least one substituent chosen from —OH, —OSO₃Q, —OPO₃Q₂, —CO₂Q, and —SO₃Q groups. In some embodiments, each R⁸, which may be identical or different, is independently chosen from C₂₋₁₂ alkenyl groups substituted with at least one substituent chosen from —OH, —OSO₃Q, —OPO₃Q₂, —CO₂Q, and —SO₃Q groups. In some embodiments, each R⁸, which may be identical or different, is independently chosen from C₁₋₄ alkyl groups substituted with at least one substituent chosen from —OH, —OSO₃Q, —OPO₃Q₂, —CO₂Q, and —SO₃Q groups. In some embodiments, each R⁸, which may be identical or different, is independently chosen from C₂₋₄ alkenyl groups substituted with at least one substituent chosen from —OH, —OSO₃Q, —OPO₃Q₂, —CO₂Q, and —SO₃Q groups. In some embodiments, each R⁸, which may be identical or different, is independently chosen from C₁₋₅ alkyl groups substituted with at least one substituent chosen from —OH, —OSO₃Q, —OPO₃Q₂, —CO₂Q, and —SO₃Q groups. In some embodiments, each R⁸, which may be identical or different, is independently chosen from C₂₋₅ alkenyl groups substituted with at least one substituent chosen from —OH, —OSO₃Q, —OPO₃Q₂, —CO₂Q, and —SO₃Q groups.

In some embodiments, each R⁸, which may be identical or different, is independently chosen from C₁₋₈ alkyl groups substituted with at least two substituents independently chosen from —OH, —OSO₃Q, —OPO₃Q₂, —CO₂Q, and —SO₃Q groups. In some embodiments, each R⁸, which may be identical or different, is independently chosen from C₂, a alkenyl groups substituted with at least two substituents independently chosen from —OH, —OSO₃Q, —OPO₃Q₂, —CO₂Q, and —SO₃Q groups. In some embodiments, each R⁸, which may be identical or different, is independently chosen from C₁₋₅ alkyl groups substituted with at least two substituents independently chosen from —OH, —OSO₃Q, —OPO₃Q₂, —CO₂Q, and —SO₃Q groups. In some embodiments, each R⁸, which may be identical or different, is independently chosen from C₂₋₅ alkenyl groups substituted with at least two substituents independently chosen from —OH, —OSO₃Q, —OPO₃Q₂, —CO₂Q, and —SO₃Q groups.

In some embodiments, each R⁸, which may be identical or different, is independently chosen from C₁₋₈ alkyl groups substituted with at least three substituents independently chosen from —OH, —OSO₃Q, —OPO₃Q₂, —CO₂Q, and —SO₃Q groups. In some embodiments, each R⁸, which may be identical or different, is independently chosen from C₂₋₈ alkenyl groups substituted with at least three substituents independently chosen from —OH, —OSO₃Q, —OPO₃Q₂, —CO₂Q, and —SO₃Q groups. In some embodiments, each R⁸, which may be identical or different, is independently chosen from C₁₋₅ alkyl groups substituted with at least three substituents independently chosen from —OH, —OSO₃Q, —OPO₃Q₂, —CO₂Q, and —SO₃Q groups. In some embodiments, each R⁸, which may be identical or different, is independently chosen from C₂₋₅ alkenyl groups substituted with at least three substituents independently chosen from —OH, —OSO₃Q, —OPO₃Q₂, —CO₂Q, and —SO₃Q groups.

In some embodiments, each R⁸, which may be identical or different, is independently chosen from

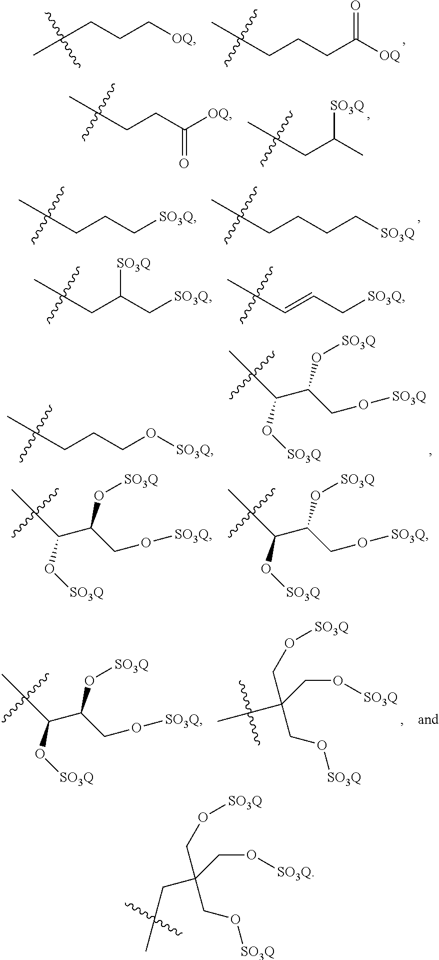

groups.

In some embodiments, each R⁸, which may be identical or different, is independently chosen from

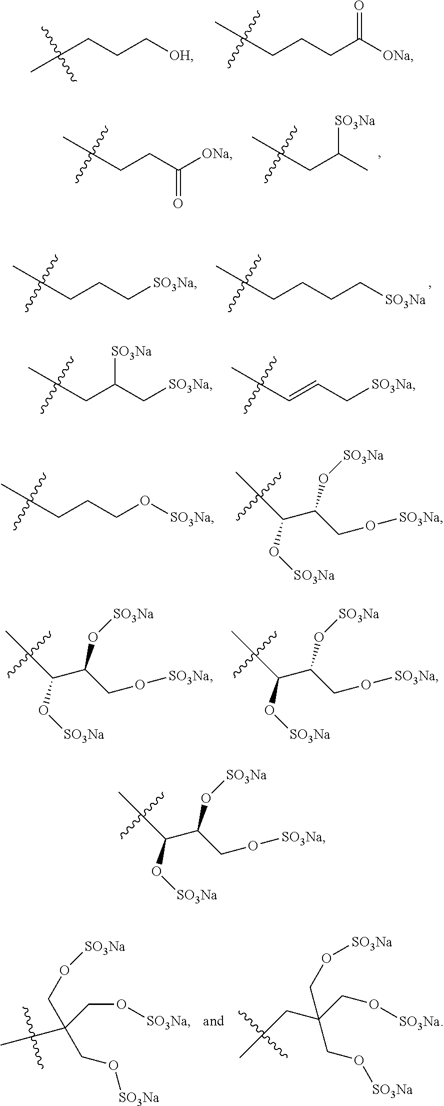

In some embodiments, each R², which may be identical or different, is independently chosen from a linker-non-glycomimetic moiety, wherein each non-glycomimetic moiety, which may be identical or different, is independently chosen from

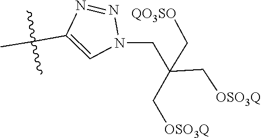

groups.

In some embodiments, each R², which may be identical or different, is independently chosen from a linker-non-glycomimetic moiety, wherein each non-glycomimetic moiety is

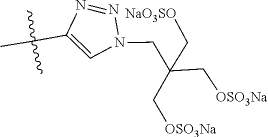

In some embodiments, each R² is identical and chosen from a linker-non-glycomimetic moiety, wherein the non-glycomimetic moiety is chosen from R⁸, C₆₋₁₈ aryl-R⁸, and C₁₋₁₂ heteroaryl-R⁸ groups. In some embodiments, each R² is identical and chosen from a linker-non-glycomimetic moiety, wherein the non-glycomimetic moiety is chosen from R⁸ groups. In some embodiments, each R² is identical and chosen from a linker-non-glycomimetic moiety, wherein the non-glycomimetic moiety is chosen from C₆₋₁₈ aryl-R⁸ groups. In some embodiments, each R² is identical and chosen from a linker-non-glycomimetic moiety, wherein the non-glycomimetic moiety is chosen from C₁₋₁₂ heteroaryl-R⁸ groups. In some embodiments, each R² is identical and chosen from a linker-non-glycomimetic moiety, wherein the non-glycomimetic moiety is chosen from

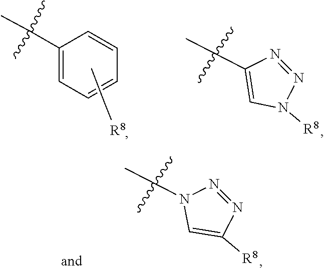

groups.

In some embodiments, each R⁸ is identical and chosen from C₁₋₁₂ alkyl groups substituted with at least one substituent chosen from —OH, —OSO₃Q, —OPO₃Q₂, —CO₂Q, and —SO₃Q groups. In some embodiments, each R⁸ is identical and chosen from C₂₋₁₂ alkenyl groups substituted with at least one substituent chosen from —OH, —OSO₃Q, —OPO₃Q₂, —CO₂Q, and —SO₃Q groups. In some embodiments, each R⁸ is identical and chosen from C₁₋₈ alkyl groups substituted with at least one substituent chosen from —OH, —OSO₃Q, —OPO₃Q₂, —CO₂Q, and —SO₃Q groups. In some embodiments, each R⁸ is identical and chosen from C₂₋₈ alkenyl groups substituted with at least one substituent chosen from —OH, —OSO₃Q, —OPO₃Q₂, —CO₂Q, and —SO₃Q groups. In some embodiments, each R⁸ is identical and chosen from C₁₋₅ alkyl groups substituted with at least one substituent chosen from —OH, —OSO₃Q, —OPO₃Q₂, —CO₂Q, and —SO₃Q groups. In some embodiments, each R⁸ is identical and chosen from C₂₋₅ alkenyl groups substituted with at least one substituent chosen from —OH, —OSO₃Q, —OPO₃Q₂, —CO₂Q, and —SO₃Q groups.

In some embodiments, each R⁸ is identical and chosen from C₁₋₈ alkyl groups substituted with at least two substituents independently chosen from —OH, —OSO₃Q, —OPO₃Q₂, —CO₂Q, and —SO₃Q groups. In some embodiments, each R⁸ is identical and chosen from C₂₋₈ alkenyl groups substituted with at least two substituents independently chosen from —OH, —OSO₃Q, —OPO₃Q₂, —CO₂Q, and —SO₃Q groups. In some embodiments, each R⁸ is identical and chosen from C₁₋₅ alkyl groups substituted with at least two substituents independently chosen from —OH, —OSO₃Q, —OPO₃Q₂, —CO₂Q, and —SO₃Q groups. In some embodiments, each R⁸ is identical and chosen from C₂₋₅ alkenyl groups substituted with at least two substituents independently chosen from —OH, —OSO₃Q, —OPO₃Q₂, —CO₂Q, and —SO₃Q groups.

In some embodiments, each R⁸ is identical and chosen from C₁₋₈ alkyl groups substituted with at least three substituents independently chosen from —OH, —OSO₃Q, —OPO₃Q₂, —CO₂Q, and —SO₃Q groups. In some embodiments, each R⁸ is identical and chosen from C₂₋₈ alkenyl groups substituted with at least three substituents independently chosen from —OH, —OSO₃Q, —OPO₃Q₂, —CO₂Q, and —SO₃Q groups. In some embodiments, each R⁸⁷ is identical and chosen from C₁₋₅ alkyl groups substituted with at least three substituents independently chosen from —OH, —OSO₃Q, —OPO₃Q₂, —CO₂Q, and —SO₃Q groups. In some embodiments, each R⁸ is identical and chosen from C₂₋₅ alkenyl groups substituted with at least three substituents independently chosen from —OH, —OSO₃Q, —OPO₃Q₂, —CO₂Q, and —SO₃Q groups.

In some embodiments, each R⁸ is identical and chosen from

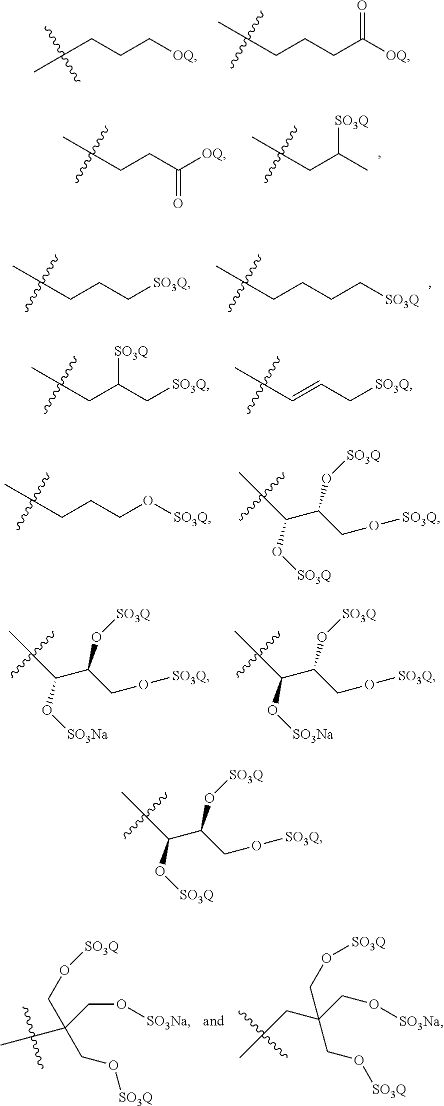

In some embodiments, each R⁸ is identical and chosen from

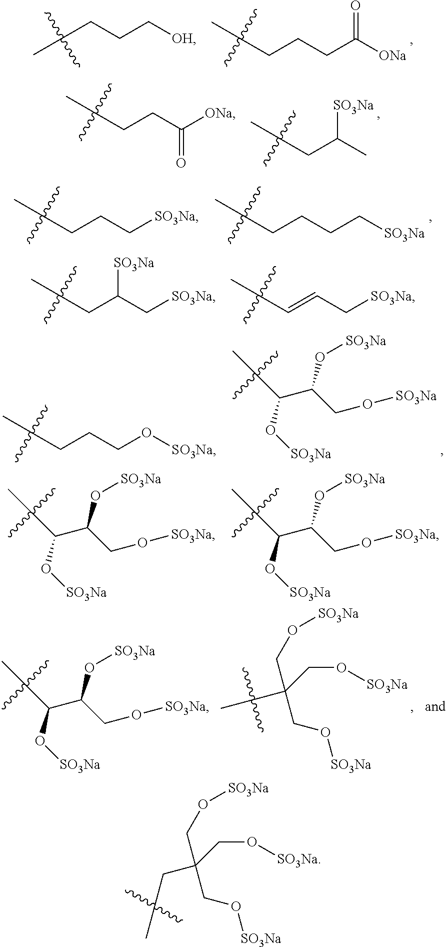

In some embodiments, each R² is identical and chosen from a linker-non-glycomimetic moiety, wherein the non-glycomimetic moiety is chosen from

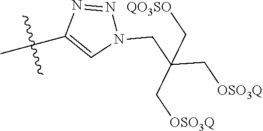

groups.

In some embodiments, each R² is identical is chosen from a linker-non-glycomimetic moiety, wherein the non-glycomimetic moiety is

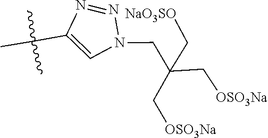

In some embodiments, the at least one substituent of R⁸ is —OH. In some embodiments, the at least one substituent of R⁸ is chosen from —OSO₃Q groups. In some embodiments, the at least one substituent of R⁸ is chosen from —OPO₃Q₂ groups. In some embodiments, the at least one substituent of R⁸ is chosen from —CO₂Q groups. In some embodiments, the at least one substituent of R⁸ is chosen from —SO₃Q groups. In some embodiments, the at least two substituents of R⁸ are —OH. In some embodiments, the at least two substituents of R⁸ are independently chosen from —OSO₃Q groups. In some embodiments, the at least two substituents of R⁸ are independently chosen from —OPO₃Q₂ groups. In some embodiments, the at least two substituents of R⁸ are independently chosen from —CO₂Q groups. In some embodiments, the at least two substituents of R⁸ are independently chosen from —SO₃Q groups. In some embodiments, the at least three substituents of R⁸ are —OH. In some embodiments, the at least three substituents of R⁸ are independently chosen from —OSO₃Q groups. In some embodiments, the at least three substituents of R⁸ are independently chosen from —OPO₃Q₂ groups. In some embodiments, the at least three substituents of R⁸ are independently chosen from —CO₂Q groups. In some embodiments, the at least three substituents of R⁸ are independently chosen from —SO₃Q groups.

In some embodiments, at least one Q is H. In some embodiments, at least one Q is chosen from pharmaceutically acceptable cations. In some embodiments, at least one Q is chosen from sodium, potassium, lithium, ammonium (substituted and unsubstituted), calcium, magnesium, iron, zinc, copper, manganese, and aluminum cations. In some embodiments, at least one Q is a sodium cation. In some embodiments, at least one Q is a potassium cation. In some embodiments, at least one Q is chosen from ammonium cations.

In some embodiments, each Q, which may be identical or different, is independently chosen from pharmaceutically acceptable cations. In some embodiments, each Q, which may be identical or different, is independently chosen from sodium, potassium, lithium, ammonium (substituted and unsubstituted), calcium, magnesium, iron, zinc, copper, manganese, and aluminum cations.

In some embodiments, each Q is H. In some embodiments, each Q is identical and independently chosen from pharmaceutically acceptable cations. In some embodiments, each Q is independently chosen from sodium, potassium, lithium, ammonium (substituted and unsubstituted), calcium, magnesium, iron, zinc, copper, manganese, and aluminum cations. In some embodiments, each Q is a sodium cation. In some embodiments, each Q is a potassium cation. In some embodiments, each Q is chosen from ammonium cations.

In some embodiments, at least one R² is chosen from a linker-non-glycomimetic moiety, wherein the non-glycomimetic moiety is chosen from polyethylene glycol (PEG), thiazolyl, and chromenyl groups.

In some embodiments, at least one R² is chosen from a linker-non-glycomimetic moiety, wherein the non-glycomimetic moiety is chosen from PEG groups. In some embodiments, at least one R² is chosen from a linker-non-glycomimetic moiety, wherein the non-glycomimetic moiety is a PEG group chosen from

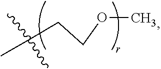

wherein r is chosen from integers ranging from 1 to 100. In some embodiments, at least one r is is an integer ranging from 1-25. In some embodiments, r is an integer ranging from 1-50. In some embodiments, r is an integer ranging from 2-15. In some embodiments, r is an integer ranging from 2-20. In some embodiments, r is an integer ranging from 2-25. In some embodiments, r is an integer ranging from 2-50. In some embodiments, r is an integer ranging from 2-100. In some embodiments, r is an integer ranging from 5-20. In some embodiments, r is an integer ranging from 5-40. In some embodiments, r is an integer ranging from 5-100. In some embodiments, r is 4. In some embodiments, r is 8. In some embodiments, r is 12. In some embodiments, r is 16. In some embodiments, r is 20. In some embodiments, r is 24. In some embodiments, r is 28.

In some embodiments, at least one R² is chosen from a linker-non-glycomimetic moiety, wherein the non-glycomimetic moiety is chosen from thiazolyl groups. In some embodiments, at least one R² is chosen from a linker-non-glycomimetic moiety, wherein the non-glycomimetic moiety is

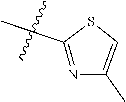 [SMILES: *c1nc(C)cs1]

In some embodiments, at least one R² is chosen from a linker-non-glycomimetic moiety, wherein the non-glycomimetic moiety is chosen from chromenyl groups. In some embodiments, at least one R² is chosen from a linker-non-glycomimetic moiety, wherein the non-glycomimetic moiety is

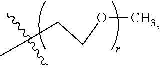

In some embodiments, each R², which may be identical or different, is independently chosen from a linker-non-glycomimetic moiety, wherein each non-glycomimetic moiety, which may be identical or different, is independently chosen from polyethylene glycol (PEG), thiazolyl, and chromenyl groups.

In some embodiments, each R², which may be identical or different, is independently chosen from a linker-non-glycomimetic moiety, wherein each non-glycomimetic moiety, which may be identical or different, is independently chosen from PEG groups. In some embodiments, each R², which may be identical or different, is independently chosen from a linker-non-glycomimetic moiety, wherein each non-glycomimetic moiety, which may be identical or different, is independently chosen from

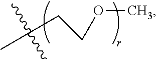

wherein each r, which may be identical or different, is independently chosen from integers ranging from 1 to 100. In some embodiments, each r, which may be identical or different, is independently chosen from integers ranging from 1-25. In some embodiments, each r, which may be identical or different, is independently chosen from integers ranging from 1-50. In some embodiments, each r, which may be identical or different, is independently chosen from integers ranging from 2-15. In some embodiments, each r, which may be identical or different, is independently chosen from integers ranging from 2-20. In some embodiments, each r, which may be identical or different, is independently chosen from integers ranging from 2-20. In some embodiments, each r, which may be identical or different, is independently chosen from integers ranging from 2-25. In some embodiments, each r, which may be identical or different, is independently chosen from integers ranging from 2-50. In some embodiments, each r, which may be identical or different, is independently chosen from integers ranging from 2-100. In some embodiments each r, which may be identical or different, is independently chosen from integers ranging from 5-20. In some embodiments, each r, which may be identical or different, is independently chosen from integers ranging from 5-40.

In some embodiments, each R², which may be identical or different, is independently chosen from a linker-non-glycomimetic moiety, wherein each non-glycomimetic moiety, which may be identical or different, is independently chosen from thiazolyl groups. In some embodiments, each R², which may be identical or different, is independently chosen from a linker-non-glycomimetic moiety, wherein the non-glycomimetic moiety is

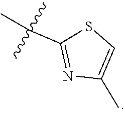 [SMILES: *c1nc(C)cs1]

In some embodiments, each R², which may be identical or different, is independently chosen from a linker-non-glycomimetic moiety, wherein each non-glycomimetic moiety, which may be identical or different, is independently chosen from chromenyl groups. In some embodiments, each R², which may be identical or different, is independently chosen from a linker-non-glycomimetic moiety, wherein each non-glycomimetic moiety is

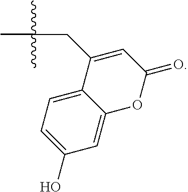 [SMILES: *Cc1cc(=O)oc2cc(O)ccc12]

In some embodiments, each R² is identical and chosen from a linker-non-glycomimetic moiety, wherein each non-glycomimetic moiety is chosen from PEG, thiazolyl, and chromenyl groups.

In some embodiments, each R² is identical and chosen from a linker-non-glycomimetic moiety, wherein the non-glycomimetic moiety is chosen from PEG groups. In some embodiments, each R² is identical and chosen from a linker-non-glycomimetic moiety, wherein the non-glycomimetic moiety is

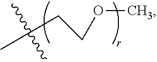

wherein each r is identical and chosen from integers ranging from 1 to 100. In some embodiments, each r is identical and chosen from integers ranging from 1-25. In some embodiments, each r is identical and chosen from integers ranging from 1-50. In some embodiments, each r is identical and chosen from integers ranging from 2-15. In some embodiments, each r is identical and chosen from integers ranging from 2-20. In some embodiments, each r is identical and chosen from integers ranging from 2-20. In some embodiments, each r is identical and chosen from integers ranging from 2-25. In some embodiments, each r is identical and chosen from integers ranging from 2-50. In some embodiments, each r is identical and chosen from integers ranging from 2-100. In some embodiments, each r is identical and chosen from integers ranging from 5-20. In some embodiments, each r is identical and chosen from integers ranging from 5-40. In some embodiments, each r is identical and chosen from integers ranging from 5-100. In some embodiments, each r is 4. In some embodiments, each r is 8. In some embodiments, each r is 12. In some embodiments, each r is 16. In some embodiments, each r is 20. In some embodiments, each r is 24. In some embodiments, each r is 28.

In some embodiments, each R² is identical and independently chosen from a linker-non-glycomimetic moiety, wherein each non-glycomimetic moiety is chosen from thiazolyl groups. In some embodiments, each R² is identical and independently chosen from a linker-non-glycomimetic moiety, wherein the non-glycomimetic moiety is

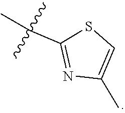 [SMILES: *c1nc(C)cs1]

In some embodiments, each R² is identical and independently chosen from a linker-non-glycomimetic moiety, wherein each non-glycomimetic moiety is chosen from chromenyl groups. In some embodiments, each R² is identical and independently chosen from a linker-non-glycomimetic moiety, wherein each non-glycomimetic moiety is

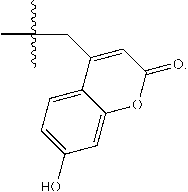 [SMILES: *Cc1cc(=O)oc2cc(O)ccc12]

In some embodiments, at least one R³ is —CN. In some embodiments, each R³ is —CN. In some embodiments, at least one R³ is —CH₂CN. In some embodiments, each R³ is —CH₂CN.

In some embodiments, at least one R³ is chosen from —C(═O)Y² groups, wherein at least one Y² is chosen from —OZ¹, —NHOH, —NHOCH₃, and —NZ¹Z² groups. In some embodiments, each R³, which may be identical or different, is independently chosen from —C(═O)Y² groups, wherein each Y², which may be identical or different, is independently chosen from —OZ¹, —NHOH, —NHOCH₃, and —NZ¹Z² groups. In some embodiments, each R³ is identical and chosen from —C(═O)Y² groups, wherein Y² is chosen from —OZ¹, —NHOH, —NHOCH₃, and —NZ¹Z² groups

In some embodiments, at least one R³ is chosen from —C(═O)OZ¹ groups. In some embodiments, each R³, which may be identical or different, is independently chosen from —C(═O)NZ¹Z² groups. In some embodiments, each R³ is identical and chosen from —C(═O)OZ¹ groups.

In some embodiments, at least one Z¹ and at least one Z², which may be identical or different, are independently chosen from H, C₁₋₈ alkyl, C₁₋₈ haloalkyl, and C₇₋₂ arylalkyl groups. In some embodiments, at least one Z¹ or at least one Z² is H. In some embodiments, at least one Z¹ and at least one Z² is H. In some embodiments, each Z¹ and each Z² is H. In some embodiments, at least one Z¹ or at least one Z² is methyl. In some embodiments, at least one Z¹ and at least one Z² is methyl. In some embodiments, each Z¹ and each Z² is methyl. In some embodiments, at least one Z¹ or at least one Z² is ethyl. In some embodiments, at least one Z¹ and at least one Z² is ethyl. In some embodiments, each Z¹ and each Z² is ethyl. In some embodiments, each Z¹ is H and each Z² is methyl. In some embodiments, each Z¹ and each Z¹ join together along with the nitrogen atom to which they are attached to form a ring.

In some embodiments, at least one R³ is chosen from

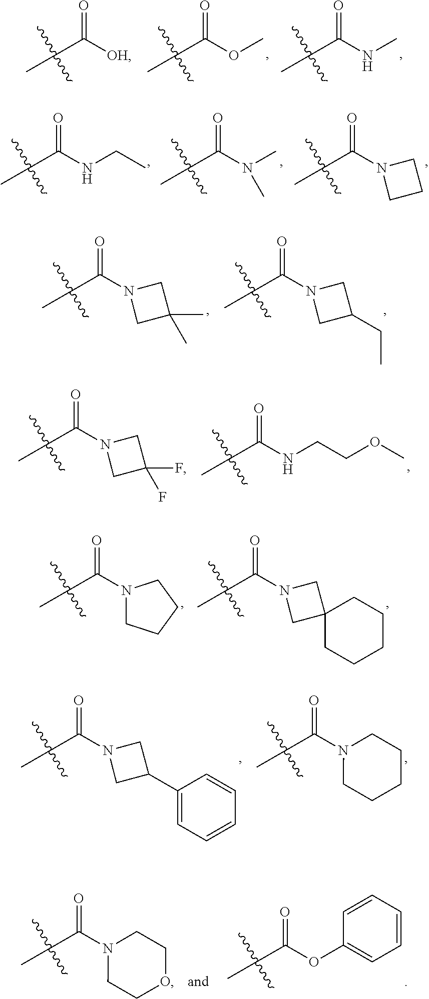

In some embodiments, at least one R³ is chosen from

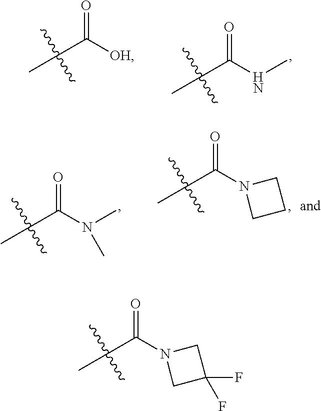

In some embodiments, at least one R³ is

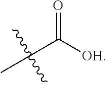 [SMILES: *C(=O)O]

In some embodiments, at least one R³ is

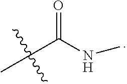 [SMILES: *C(=O)NC]

In some embodiments, at least one R³ is

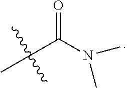 [SMILES: *C(=O)N(C)C]

In some embodiments, at least one R³ is

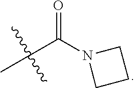 [SMILES: *C(=O)N1CCC1]

In some embodiments, at least one R³ is

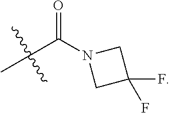 [SMILES: *C(=O)N1CC(F)(F)C1]

In some embodiments, at least one R³ is

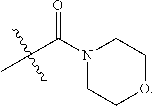 [SMILES: *C(=O)N1CCOCC1]

In some embodiments, each R³ is chosen from

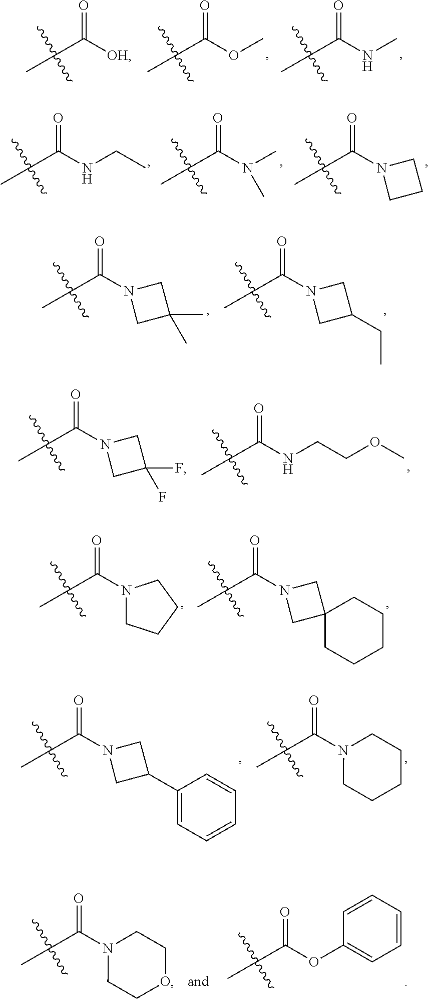

In some embodiments, each R³ is

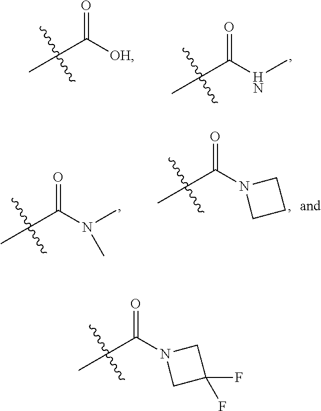

In some embodiments, each R³ is

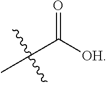 [SMILES: *C(=O)O]

In some embodiments, each R³ is

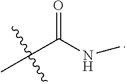 [SMILES: *C(=O)NC]

In some embodiments, each R³ is

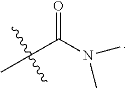 [SMILES: *C(=O)N(C)C]

In some embodiments, each R³ is

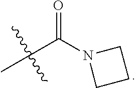 [SMILES: *C(=O)N1CCC1]

In some embodiments, each R³ is

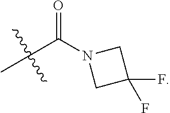 [SMILES: *C(=O)N1CC(F)(F)C1]

In some embodiments, each R³ is

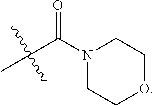 [SMILES: *C(=O)N1CCOCC1]

In some embodiments, at least one R⁴ is chosen from C₁₋₁₂ alkyl groups. In some embodiments, at least one R⁴ is chosen from C₁₋₈ alkyl groups. In some embodiments, at least one R⁴ is chosen from C₁₋₁₂ haloalkyl groups. In some embodiments, at least one R⁴ is chosen from C₁₋₈ haloalkyl groups. In some embodiments, at least one R⁴ is chosen from C₄₋₁₆ cycloalkylalkyl groups. In some embodiments, at least one R⁴ is chosen from C₄₋₈ cycloalkylalkyl groups. In some embodiments, at least one R⁴ is chosen from propyl, cyclopropylmethyl, and cyclohexylmethyl. In some embodiments, at least one R⁴ is propyl. In some embodiments, at least one R⁴ is cyclopropylmethyl. In some embodiments, at least one R⁴ is cyclohexylmethyl.

In some embodiments, each R⁴, which may be identical or different, is independently chosen from C₁₋₁₂ alkyl groups. In some embodiments, each R⁴, which may be identical or different, is independently chosen from C₁₋₈ alkyl groups. In some embodiments, each R⁴, which may be identical or different, is independently chosen from C₁₋₁₂ haloalkyl groups. In some embodiments, each R⁴, which may be identical or different, is independently chosen from C₁₋₈ haloalkyl groups. In some embodiments, each R⁴, which may be identical or different, is independently chosen from C₄₋₁₆ cycloalkylalkyl groups. In some embodiments, each R⁴, which may be identical or different, is independently chosen from C₄₋₈ cycloalkylalkyl groups. In some embodiments, each R⁴, which may be identical or different, is independently chosen from propyl, cyclopropylmethyl, and cyclohexylmethyl.

In some embodiments, each R⁴ is identical and chosen from C₁₋₁₂ alkyl groups. In some embodiments, each R⁴ is identical and chosen from C₁₋₈ alkyl groups. In some embodiments, each R⁴ is identical and chosen from C₁₋₁₂ haloalkyl groups. In some embodiments, each R⁴ is identical and chosen from C₁₋₈ haloalkyl groups. In some embodiments, each R⁴ is identical and chosen from C₄₋₁₆ cycloalkylalkyl groups. In some embodiments, each R⁴ is identical and chosen from C₄₋₈ cycloalkylalkyl groups. In some embodiments, each R⁴ is identical and chosen from propyl, cyclopropylmethyl, and cyclohexylmethyl. In some embodiments, each R⁴ is propyl. In some embodiments, each R⁴ is cyclopropylmethyl. In some embodiments, each R⁴ is cyclohexylmethyl.

In some embodiments, at least one R⁵ is chosen from C₁₋₁₂ alkyl groups. In some embodiments, at least one R⁵ is chosen from C₁₋₈ alkyl groups. In some embodiments, at least one R⁵ is chosen from C₁₋₄ alkyl groups. In some embodiments, at least one R⁵ is chosen from C₁₋₄ haloalkyl groups. In some embodiments, at least one R⁵ is chosen from halomethyl groups. In some embodiments, at least one R⁵ is independently chosen from CF₃, CH₃, and CN. In some embodiments, at least one R⁵ is CF₃. In some embodiments, at least one R⁵ is CH₃. In some embodiments, at least one R⁵ is CN.

In some embodiments, each R, which may be identical or different, is independently chosen from C₁₋₁₂ alkyl groups. In some embodiments, each R³, which may be identical or different, is independently chosen from C₁₋₈ alkyl groups. In some embodiments, each R³, which may be identical or different, is independently chosen from C₁₋₄ alkyl groups. In some embodiments, each R, which may be identical or different, is independently chosen from C₁₋₄ haloalkyl groups. In some embodiments, each R⁵, which may be identical or different, is independently chosen from halomethyl groups. In some embodiments, each R⁵, which may be identical or different, is independently chosen from CF₃, CH₃, and CN.

In some embodiments, each R⁵ is identical and chosen from C₁₋₁₂ alkyl groups. In some embodiments, each R⁵ is identical and chosen from C₁₋₈ alkyl groups. In some embodiments, each R⁵ is identical and chosen from C₁₋₄ alkyl groups. In some embodiments, each R⁵ is identical and chosen from C₁₋₄ haloalkyl groups. In some embodiments, each R⁵ is identical and chosen from halomethyl groups. In some embodiments, each R⁵ is identical and chosen from CF₃, CH₃, and CN. In some embodiments, each R⁵ is CF₃. In some embodiments, each R⁵ is CH₃. In some embodiments, each R⁵ is CN.

In some embodiments, at least one X is —O—. In some embodiments, at least one X is

—N(R⁹)—. In some embodiments, at least one R⁹ is chosen from H and C₁₋₄ alkyl groups. In some embodiments, at least one X is —NH—.

In some embodiments, each X is —O—. In some embodiments, each X is identical and chosen from —N(R⁹)— groups. In some embodiments, each X is —NH—.

In some embodiments, m is chosen from integers ranging from 2 to 256. In some embodiments, m is chosen from integers ranging from 2 to 128. In some embodiments, m is chosen from integers ranging from 2 to 64. In some embodiments, m is chosen from integers ranging from 2 to 32. In some embodiments, m is chosen from integers ranging from 2 to 16. In some embodiments, m is chosen from integers ranging from 2 to 8. In some embodiments, m is chosen from integers ranging from 2 to 4. In some embodiments, m is 4 In some embodiments, m is 3. In some embodiments, m is 2.

In some embodiments, at least one linker groups is chosen from groups comprising spacer groups, such spacer groups as, for example, —(CH₂)_{z}— and —O(CH₂)_{z}—, wherein z is chosen from integers ranging from 1 to 250. Other non-limiting examples of spacer groups include carbonyl groups and carbonyl-containing groups such as, for example, amide groups. A non-limiting example of a spacer group is

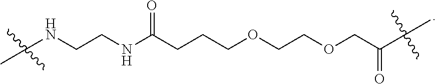 [SMILES: *NCCNC(=O)CCCOCCOCC(*)=O]

In some embodiments, at least one linker group is chosen from

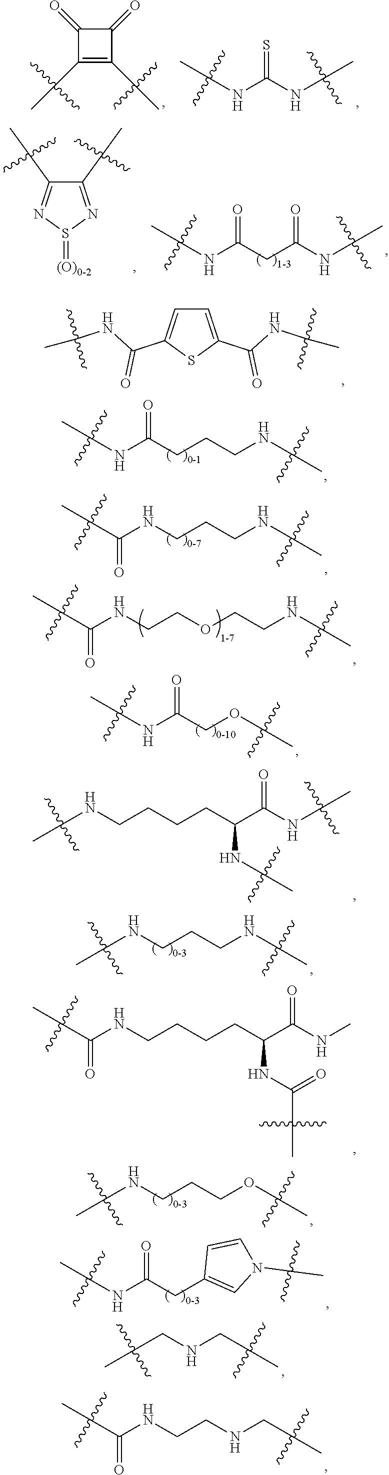

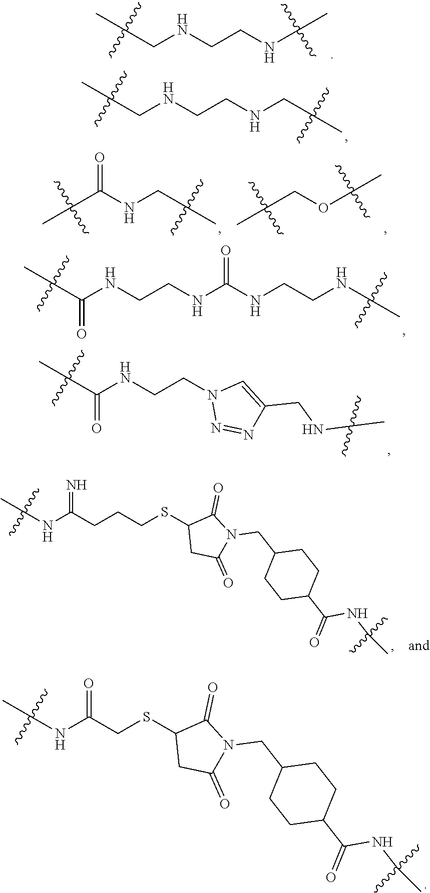

groups.

Other linker groups, such as, for example, polyethylene glycols (PEGs) and —C(═O)—NH—(CH₂)_{z}—C(═O)—NH—, wherein z is chosen from integers ranging from 1 to 250, will be familiar to those of ordinary skill in the art and/or those in possession of the present disclosure.

In some embodiments, at least one linker group is

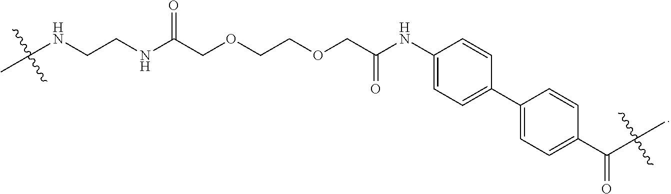 [SMILES: *NCCNC(=O)COCCOCC(=O)Nc1ccc(-c2ccc(C(*)=O)cc2)cc1]

In some embodiments, at least one linker group is

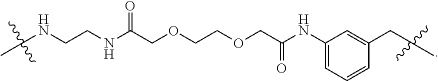 [SMILES: *Cc1cccc(NC(=O)COCCOCC(=O)NCCN*)c1]

In some embodiments, at least one linker group is chosen from —C(═O)NH(CH₂)₂NH—, —CH₂NHCH₂—, and —C(═O)NHCH₂—. In some embodiments, at least one linker group is —C(═O)NH(CH₂)₂NH—.

In some embodiments, L is chosen from dendrimers. In some embodiments, L is chosen from polyamidoamine (“PAMAM”) dendrimers. In some embodiments, L is chosen from PAMAM dendrimers comprising succinamic. In some embodiments, L is PAMAM GO generating a tetramer. In some embodiments, L is PAMAM G1 generating an octamer. In some embodiments, L is PAMAM G2 generating a 16-mer. In some embodiments, L is PAMAM G3 generating a 32-mer. In some embodiments, L is PAMAM G4 generating a 64-mer. In some embodiments, L is PAMAM G5 generating a 128-mer.

In some embodiments, m is 2 and L is chosen from

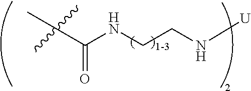

groups
wherein U is chosen from

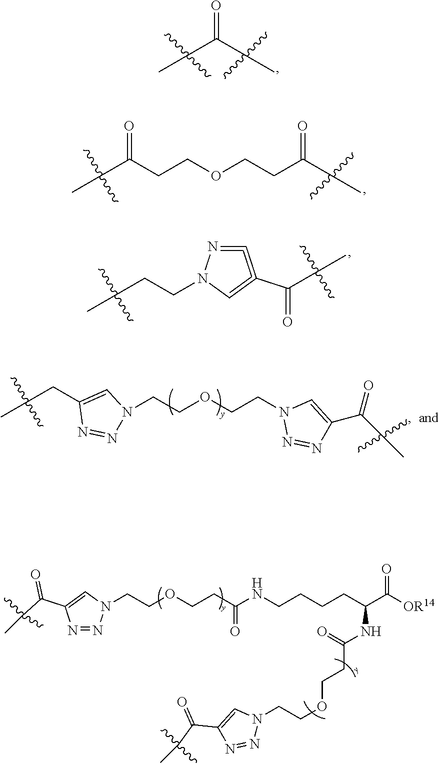

groups wherein R¹⁴ is chosen from H, C₁₋₈ alkyl, C₆₋₁₈ aryl, C₇₋₁₉ arylalkyl, and C₁₋₁₃ heteroaryl groups and each y, which may be identical or different, is independently chosen from integers ranging from 0 to 250. In some embodiments, R¹⁴ is chosen from C₁₋₈ alkyl. In some embodiments, R¹⁴ is chosen from C₇₋₁₉ arylalkyl. In some embodiments, R¹⁴ is H. In some embodiments, R¹⁴ is benzyl.

In some embodiments, L is chosen from

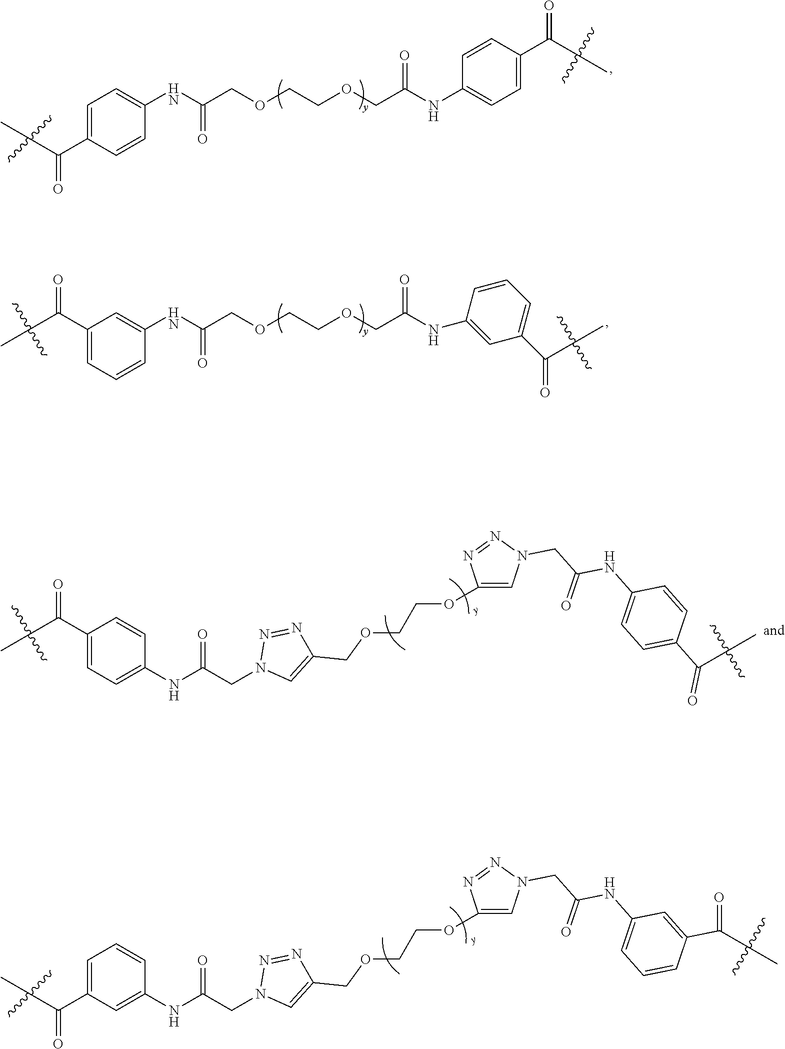

wherein y is chosen from integers ranging from 0 to 250.

In some embodiments, L is chosen from

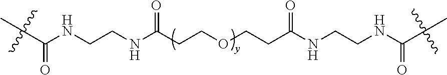

groups wherein y is chosen from integers ranging from 0 to 250.

In some embodiments, L is

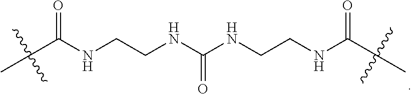 [SMILES: *C(=O)NCCNC(=O)NCCNC(*)=O]

In some embodiments, L is chosen from

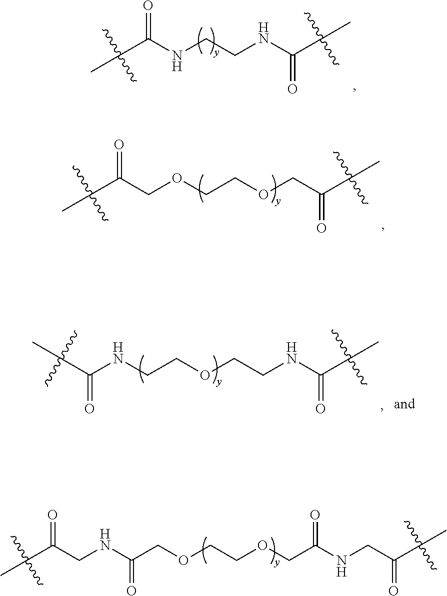

groups wherein y is chosen from integers ranging from 0 to 250.

In some embodiments, L is chosen from

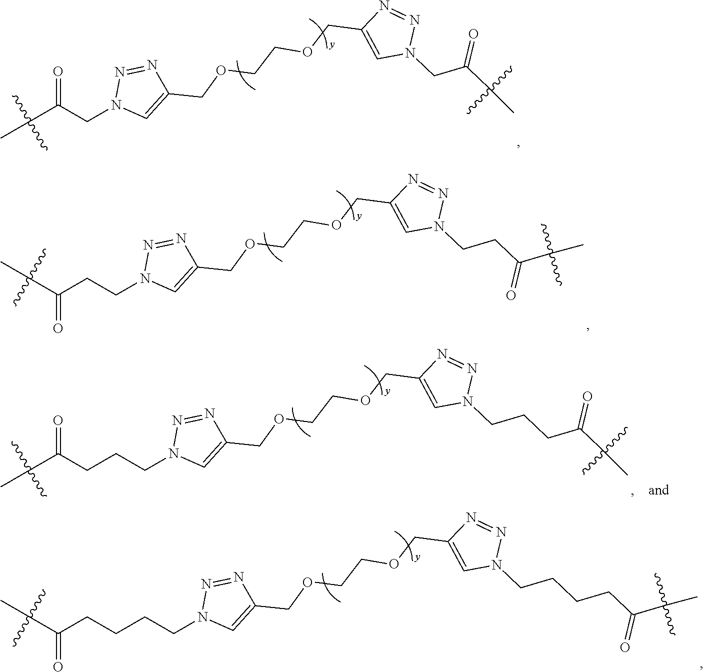

groups wherein y is chosen from integers ranging from 0 to 250.

In some embodiments, L is chosen from

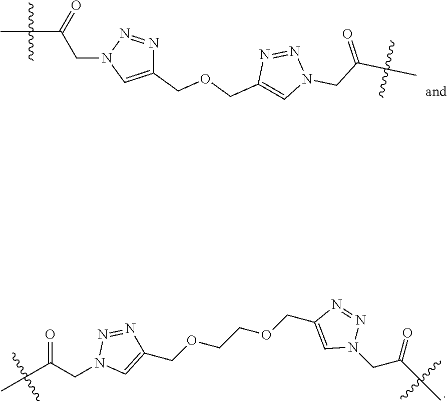 [SMILES: *C(=O)Cn1cc(COCc2cn(CC(*)=O)nn2)nn1 ; *C(=O)Cn1cc(COCCOCc2cn(CC(*)=O)nn2)nn1]

In some embodiments, L is

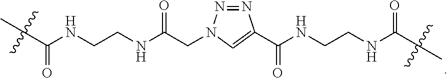 [SMILES: *C(=O)NCCNC(=O)Cn1cc(C(=O)NCCNC(*)=O)nn1]

In some embodiments, L is chosen from

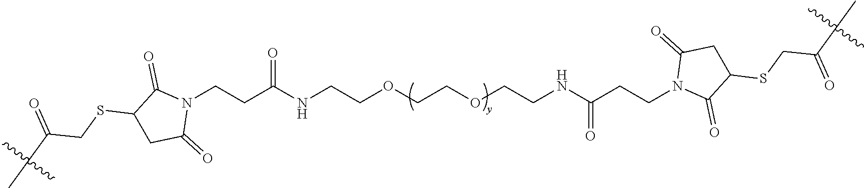

groups wherein y is chosen from integers ranging from 0 to 250.

In some embodiments, L is

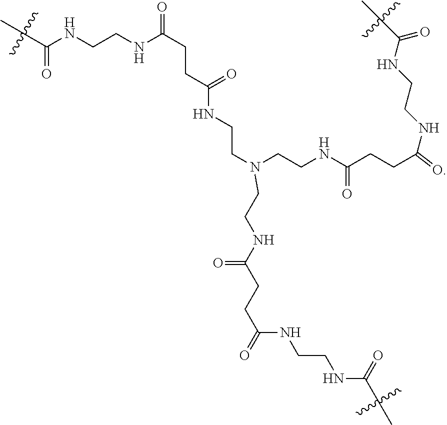 [SMILES: *C(=O)NCCNC(=O)CCC(=O)NCCN(CCNC(=O)CCC(=O)NCCNC(*)=O)CCNC(=O)CCC(=O)NCCNC(*)=O]

In some embodiments, L is

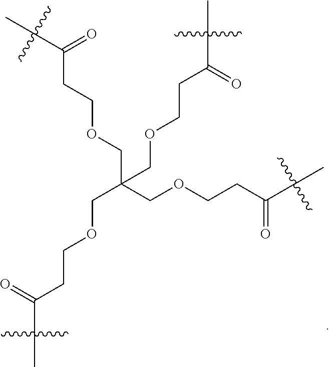 [SMILES: *C(=O)CCOCC(COCCC(*)=O)(COCCC(*)=O)COCCC(*)=O]

In some embodiments, L is

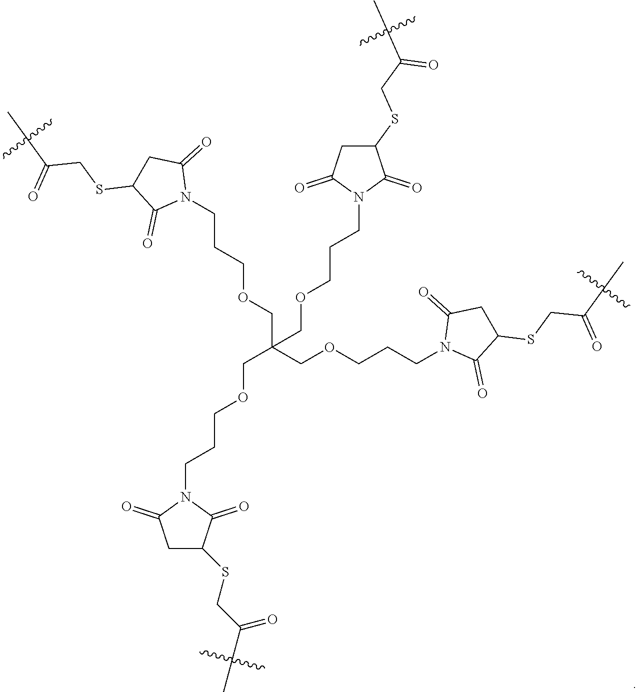 [SMILES: *C(=O)CSC1CC(=O)N(CCCOCC(COCCCN2C(=O)CC(SCC(*)=O)C2=O)(COCCCN2C(=O)CC(SCC(*)=O)C2=O)COCCCN2C(=O)CC(SCC(*)=O)C2=O)C1=O]

In some embodiments, L is chosen from

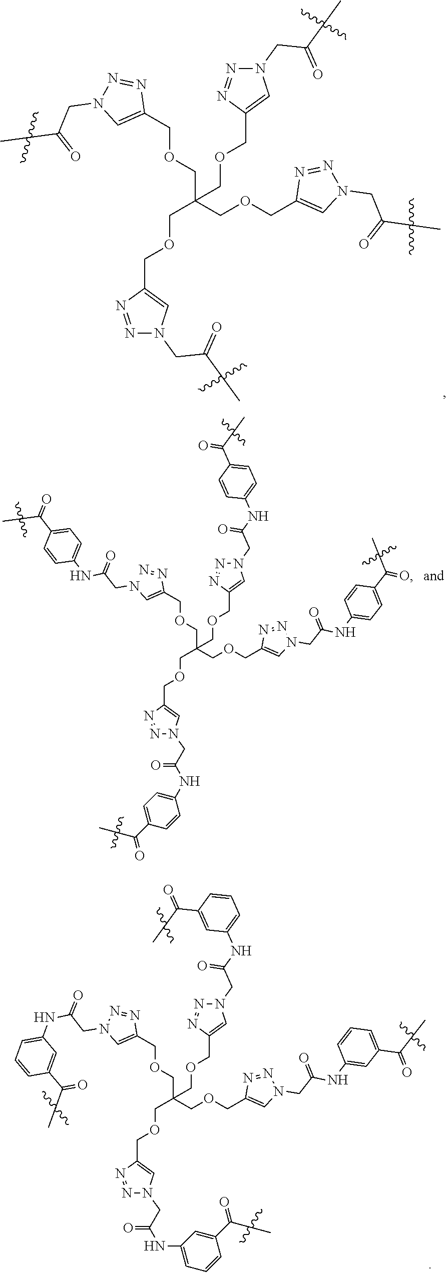 [SMILES: *C(=O)Cn1cc(COCC(COCc2cn(CC(*)=O)nn2)(COCc2cn(CC(*)=O)nn2)COCc2cn(CC(*)=O)nn2)nn1 ; *C(=O)c1ccc(NC(=O)Cn2cc(COCC(COCc3cn(CC(=O)Nc4ccc(C(*)=O)cc4)nn3)(COCc3cn(CC(=O)Nc4ccc(C(*)=O)cc4)nn3)COCc3cn(CC(=O)Nc4ccc(C(*)=O)cc4)nn3)nn2)cc1 ; "and" ; *C(=O)c1cccc(NC(=O)Cn2cc(COCC(COCc3cn(CC(=O)Nc4cccc(C(*)=O)c4)nn3)(COCc3cn(CC(=O)Nc4cccc(C(*)=O)c4)nn3)COCc3cn(CC(=O)Nc4cccc(C(*)=O)c4)nn3)nn2)c1]

In some embodiments, L is

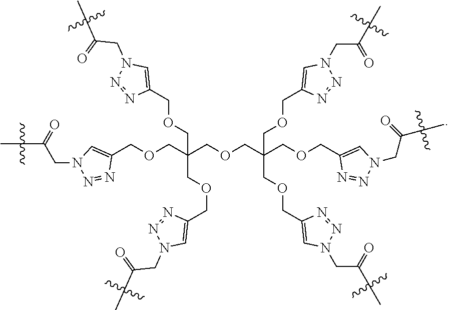 [SMILES: *C(=O)Cn1cc(COCC(COCc2cn(CC(*)=O)nn2)(COCc2cn(CC(*)=O)nn2)COCC(COCc2cn(CC(*)=O)nn2)(COCc2cn(CC(*)=O)nn2)COCc2cn(CC(*)=O)nn2)nn1]

In some embodiments, L is chosen from

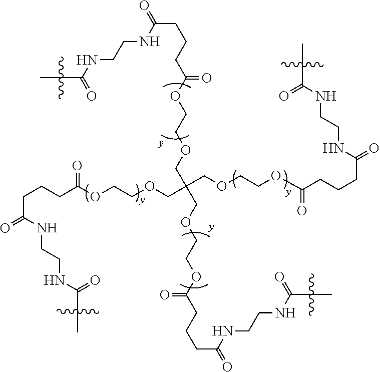

groups wherein each y, which may be identical or different, is independently chosen from integers ranging from 0 to 250.

In some embodiments, L is chosen from

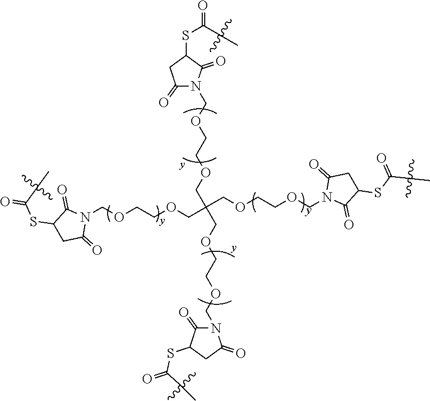

wherein each y, which may be identical or different, is independently chosen from integers ranging from 0 to 250.

In some embodiments, L is chosen from

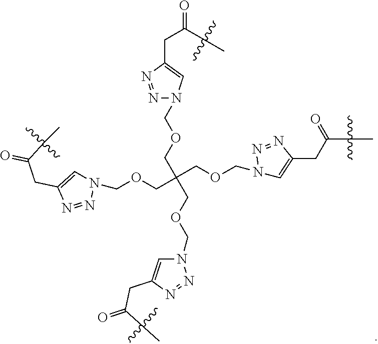 [SMILES: *C(=O)Cc1cn(COCC(COCn2cc(CC(*)=O)nn2)(COCn2cc(CC(*)=O)nn2)COCn2cc(CC(*)=O)nn2)nn1]

In some embodiments y is chosen from integers ranging from 0 to 200. In some embodiments, y is chosen from integers ranging from 0 to 150. In some embodiments, y is chosen from integers ranging from 0 to 100. In some embodiments, y is chosen from integers ranging from 0 to 50. In some embodiments, y is chosen from integers ranging from 0 to 30. In some embodiments, y is chosen from integers ranging from 0 to 15. In some embodiments, y is chosen from integers ranging from 0 to 10. In some embodiments, y is chosen from integers ranging from 0 to 5. In some embodiments, y is 117. In some embodiments, y is 25. In some embodiments, y is 21. In some embodiments, y is 17. In some embodiments y is 13. In some embodiments, y is 10. In some embodiments, y is 8. In some embodiments, y is 6. In some embodiments, y is 5. In some embodiments, y is 4. In some embodiments, y is 3. In some embodiments, y is 2. In some embodiments, y is 1. In some embodiments, y is 0.

In some embodiments, at least one compound is chosen from compounds of Formula (I), wherein each R¹ is identical, each R² is identical, each R³ is identical, each R⁴ is identical, each R⁵ is identical, and each X is identical. In some embodiments, at least one compound is chosen from compounds of Formula (I), wherein said compound is symmetrical.

In some embodiments, at least one compound is chosen from compounds having the following Formula:

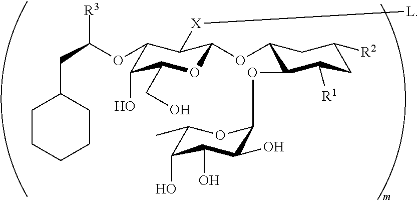

In some embodiments, at least one compound is chosen from compounds having the following Formula:

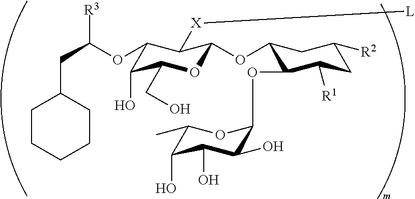

wherein each R₁, which may be identical or different, is independently chosen from methyl, ethyl, and

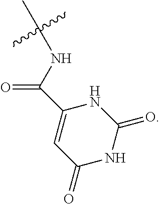 [SMILES: *NC(=O)c1cc(=O)[nH]c(=O)[nH]1]

In some embodiments, at least one compound is chosen from compounds having the following Formula:

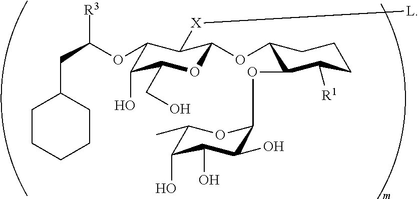

In some embodiments, at least one compound is chosen from compounds having the following Formula:

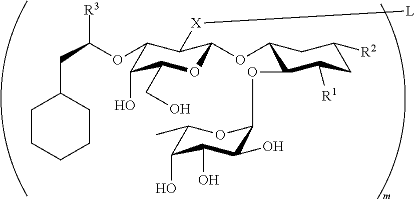

wherein each R², which may be identical or different, is independently chosen from a non-glycomimetic moiety.

In some embodiments, at least one compound is chosen from compounds having the following Formula:

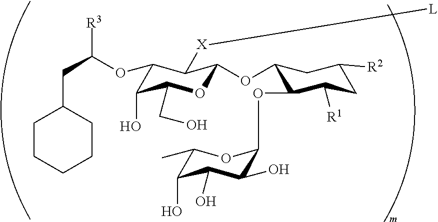

wherein each R², which may be identical or different, is independently chosen from

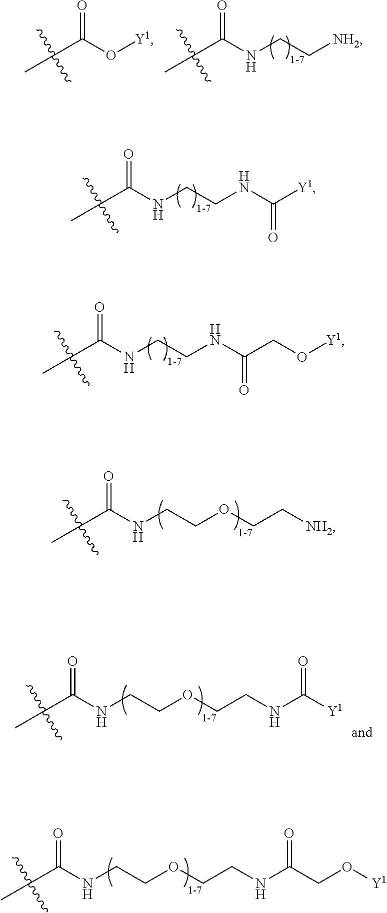

groups.

In some embodiments, at least one compound is chosen from compounds having the following Formula:

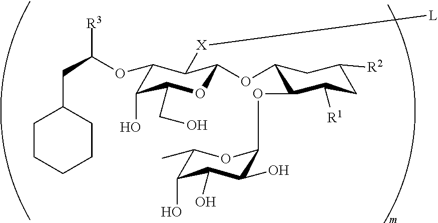

wherein each R², which may be identical or different, is independently chosen from a linker-non-glycomimetic moiety, wherein the non-glycomimetic moiety is chosen from galectin-3 inhibitors.

In some embodiments, at least one compound is chosen from compounds having the following Formula:

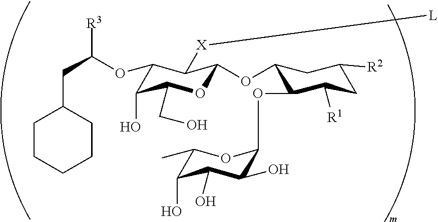

wherein each R², which may be identical or different, is independently chosen from a linker-non-glycomimetic moiety, wherein the non-glycomimetic moiety is chosen from

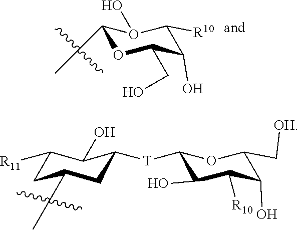

In some embodiments, at least one compound is chosen from compounds having the following Formula:

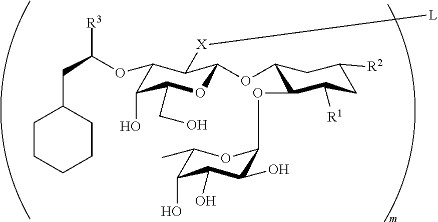

wherein each R², which may be identical or different, is independently chosen from a linker-non-glycomimetic moiety, wherein the non-glycomimetic moiety is

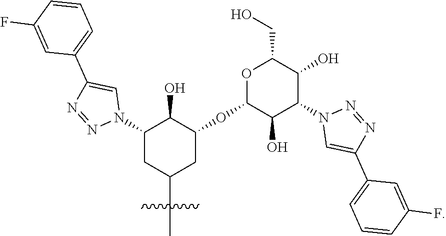 [SMILES: *C1C[C@@H](O[C@@H]2O[C@H](CO)[C@H](O)[C@H](n3cc(-c4cccc(F)c4)nn3)[C@H]2O)[C@H](O)[C@@H](n2cc(-c3cccc(F)c3)nn2)C1]

In some embodiments, at least one compound is chosen from compounds having the following Formula:

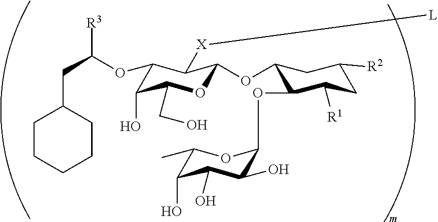

wherein each R², which may be identical or different, is independently chosen from a linker-non-glycomimetic moiety, wherein the non-glycomimetic moiety is chosen from

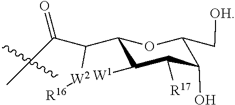

In some embodiments, at least one compound is chosen from compounds having the following Formula:

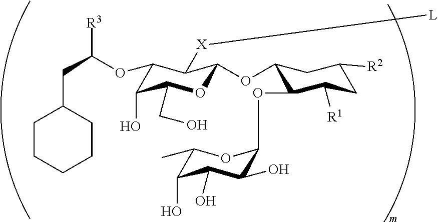

wherein each R², which may be identical or different, is independently chosen from a linker-non-glycomimetic moiety, wherein the non-glycomimetic moiety is chosen from

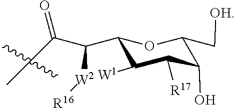

In some embodiments, at least one compound is chosen from compounds having the following Formula:

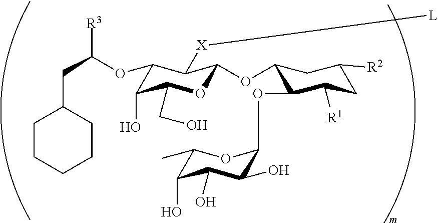

wherein each R², which may be identical or different, is independently chosen from a linker-non-glycomimetic moiety, wherein the non-glycomimetic moiety is chosen from

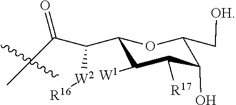

In some embodiments, at least one compound is chosen from compounds having the following Formulae:

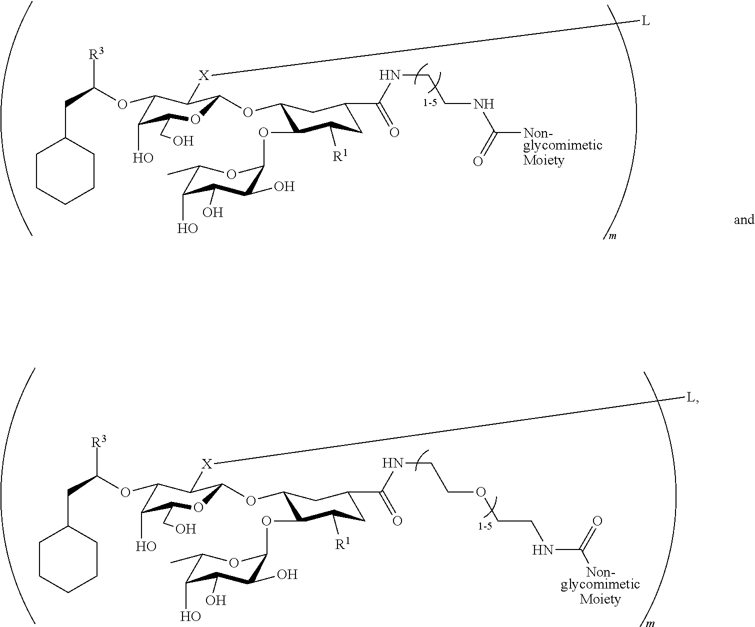

wherein each nonglycomimetic moiety, which may be identical or different, is independently chosen from galectin-3 inhibitors.

In some embodiments, at least one compound is chosen from compounds having the following Formulae:

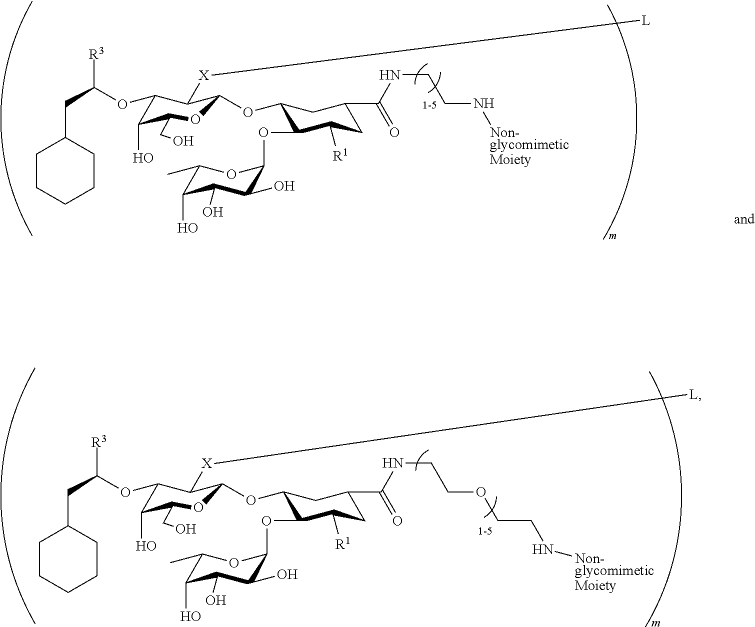

wherein each nonglycomimetic moiety, which may be identical or different, is independently chosen from galectin-3 inhibitors.

In some embodiments, at least one compound is chosen from compounds having the following Formulae:

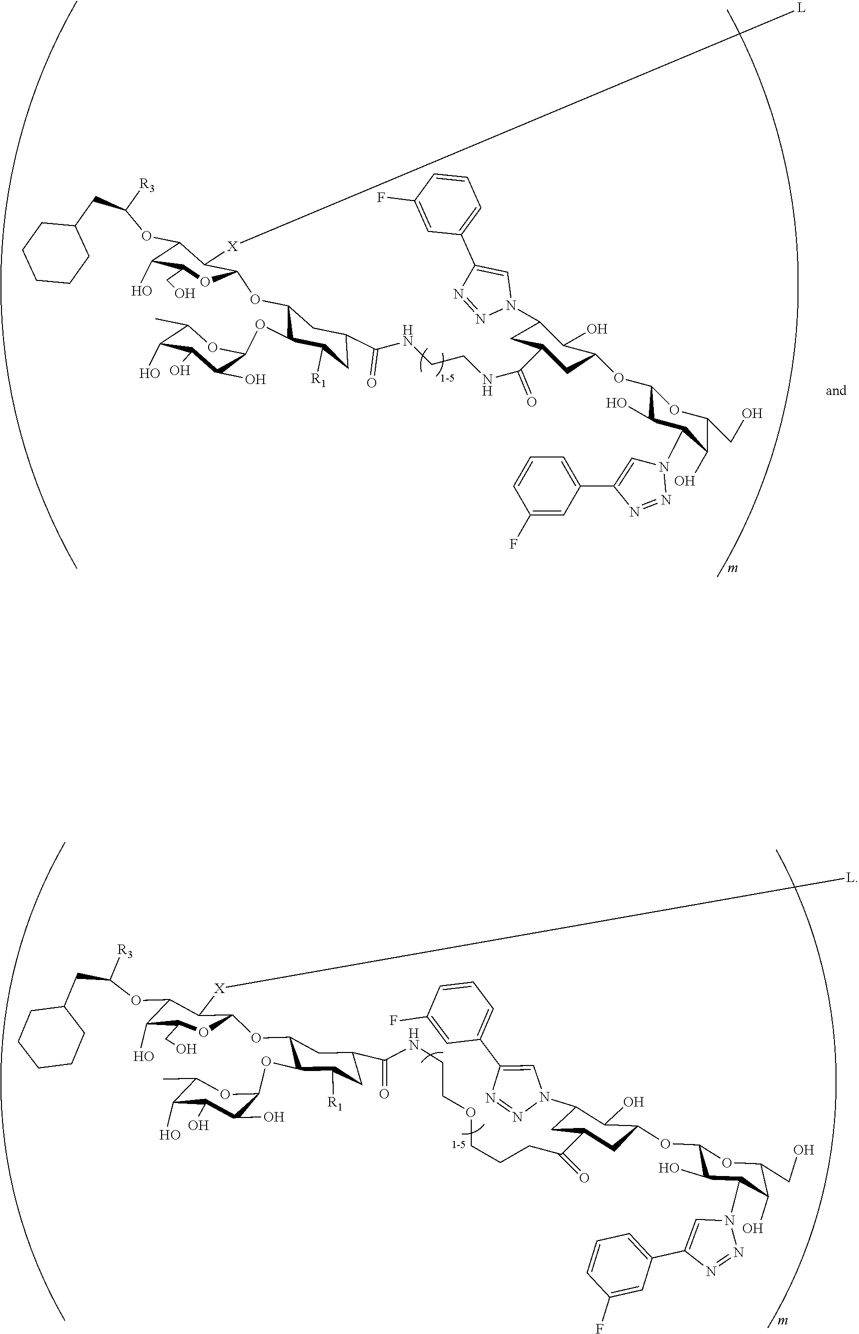

In some embodiments, at least one compound is chosen from compounds having the following Formulae:

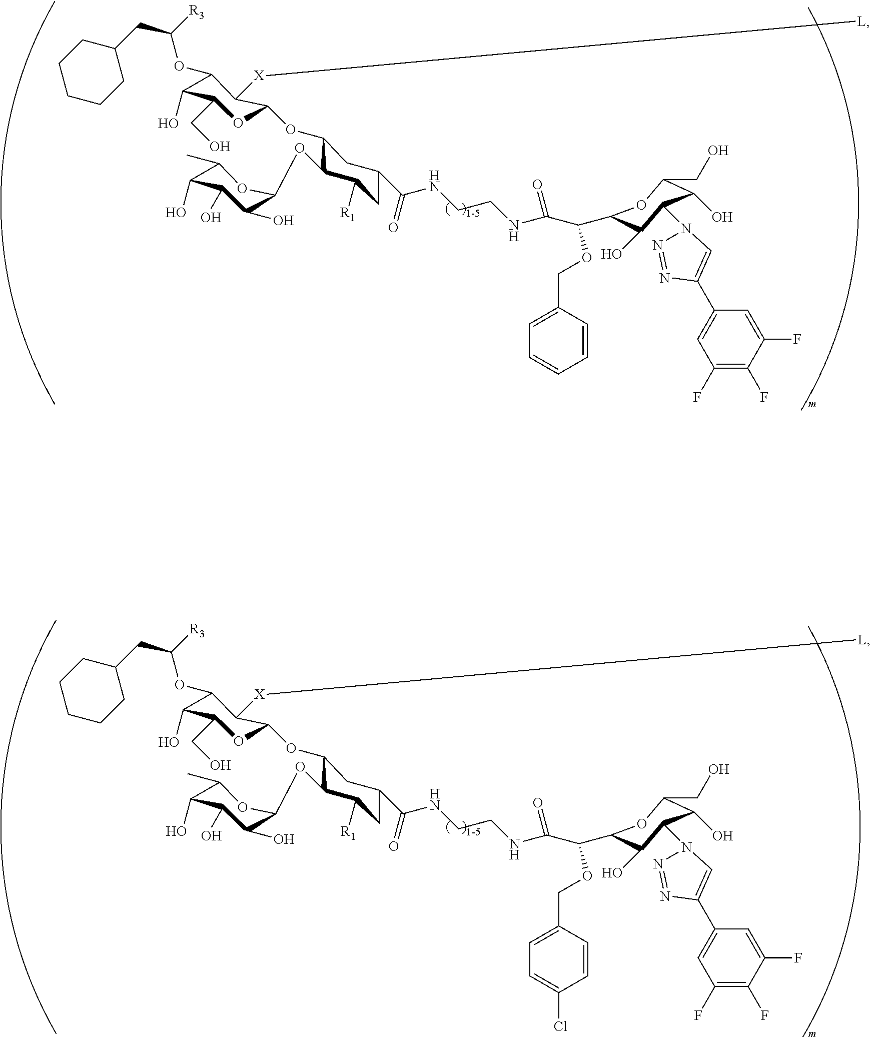

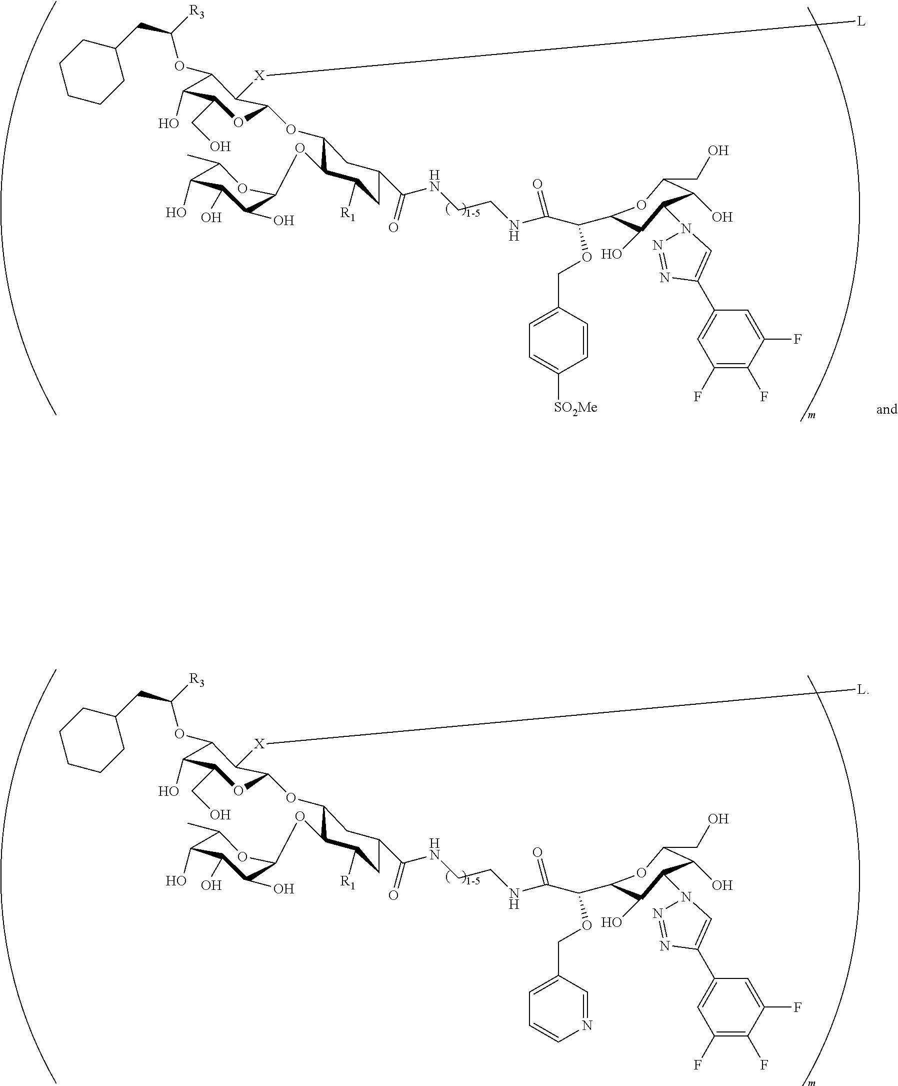

In some embodiments, at least one compound is chosen from compounds having the following Formula:

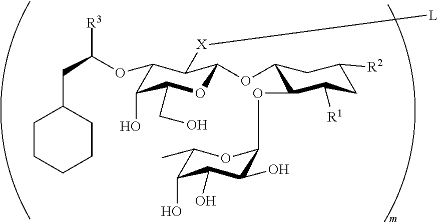

wherein each R², which may be identical or different, is independently chosen from a linker-non-glycomimetic moiety, wherein the non-glycomimetic moiety, which may be identical or different, is independently chosen from CXCR4 chemokine receptor inhibitors.

In some embodiments, at least one compound is chosen from compounds having the following Formulae:

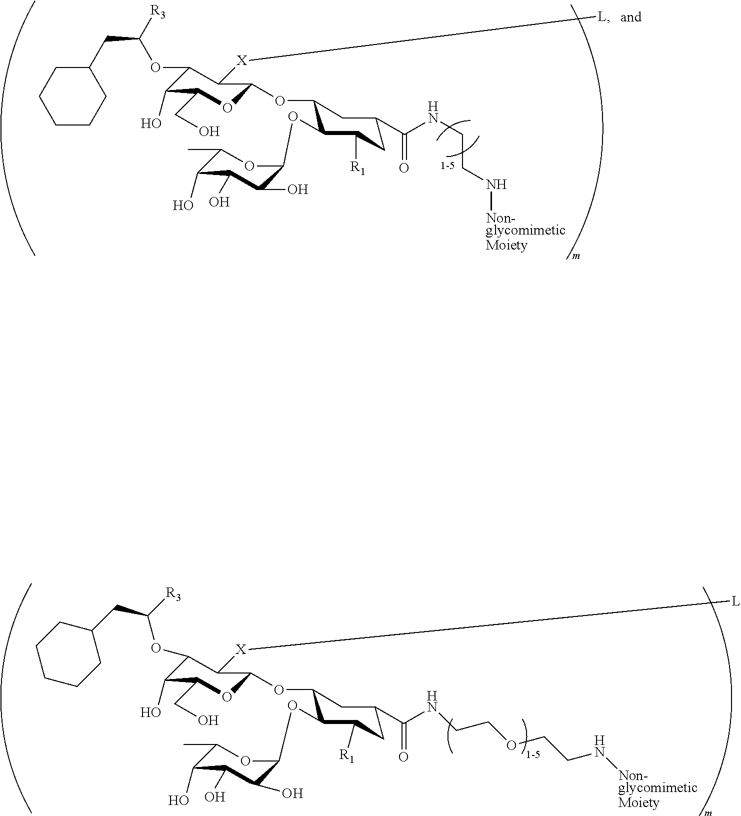

wherein each non-glycomimetic moiety, which may be identical or different, is independently chosen from CXCR4 chemokine receptor inhibitors.

In some embodiments, at least one compound is chosen from compounds having the following Formula:

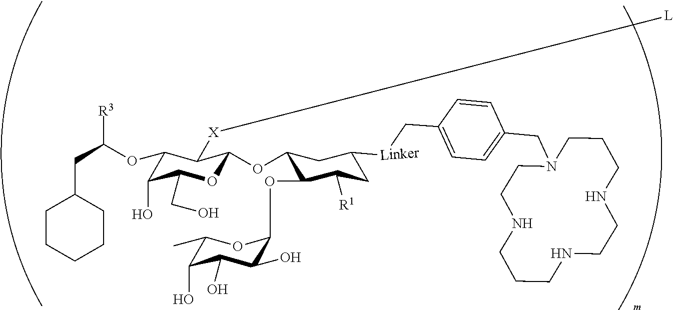

In some embodiments, at least one compound is chosen from compounds having the following Formula:

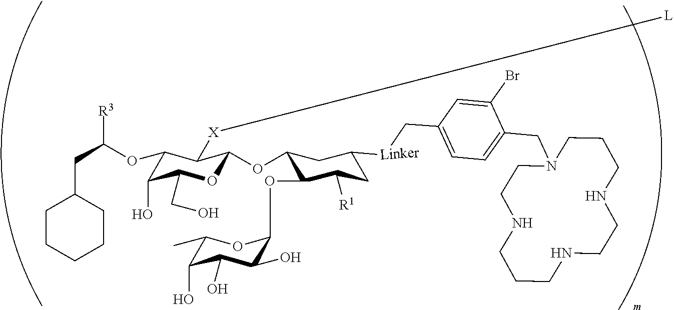

In some embodiments, at least one compound is chosen from compounds having the following Formulae:

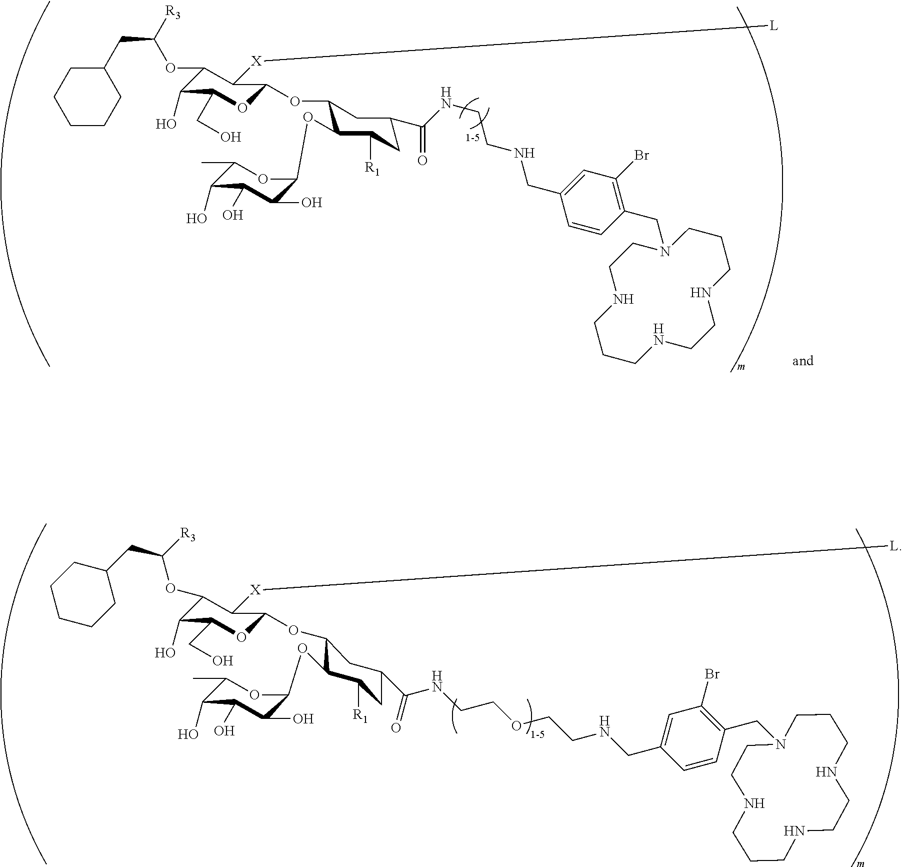

In some embodiments, at least one compound is chosen from compounds having the following Formula:

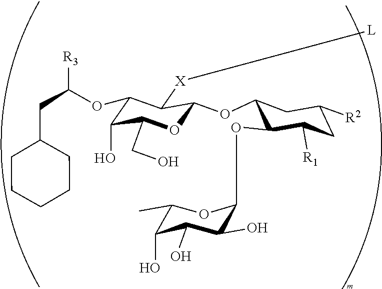

wherein each R², which may be identical or different, is independently chosen from a linker-non-glycomimetic moiety, wherein the non-glycomimetic moiety, which may be identical or different, is independently chosen from R⁸, C₆₋₁₈ aryl-R⁸, and C₁₋₁₂ heteroaryl-R⁸ groups. In some embodiments, each non-glycomimetic moiety, which may be identical or different, is independently chosen from R⁸. In some embodiments, each non-glycomimetic moiety, which may be identical or different, is independently chosen from C₆₋₁₈ aryl-R⁸. In some embodiments, each non-glycomimetic moiety, which may be identical or different, is independently chosen from C₁₋₁₂ heteroaryl-R⁸ groups.

In some embodiments, at least one compound is chosen from compounds having the following Formulae:

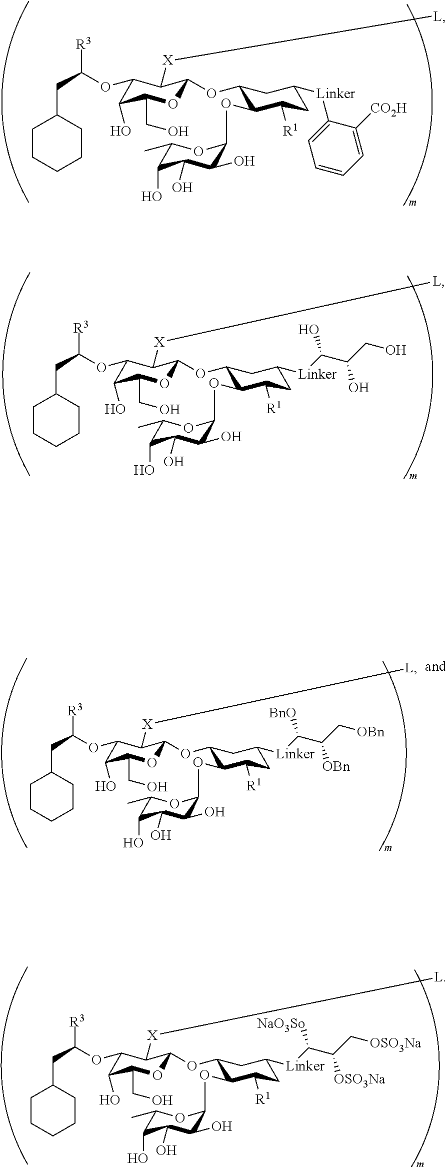

In some embodiments, at least one compound is chosen from compounds having the following Formula:

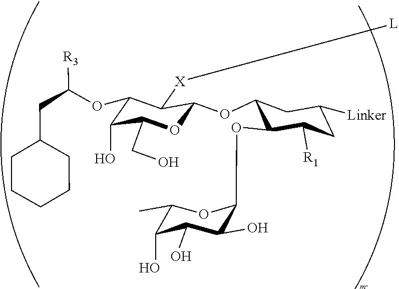

In some embodiments, at least one compound is chosen from compounds having the following Formula:

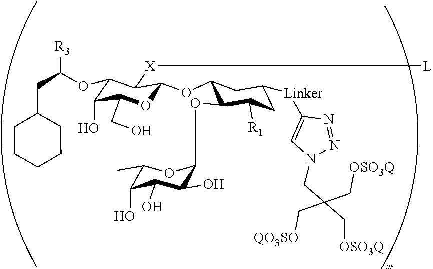

In some embodiments, at least one compound is chosen from compounds having the following Formula:

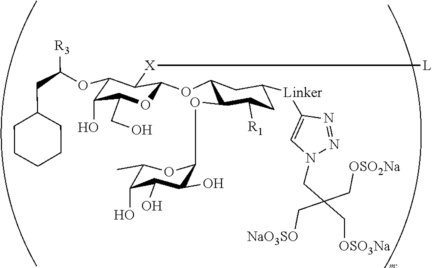

In some embodiments, at least one compound is chosen from compounds having the following Formulae:

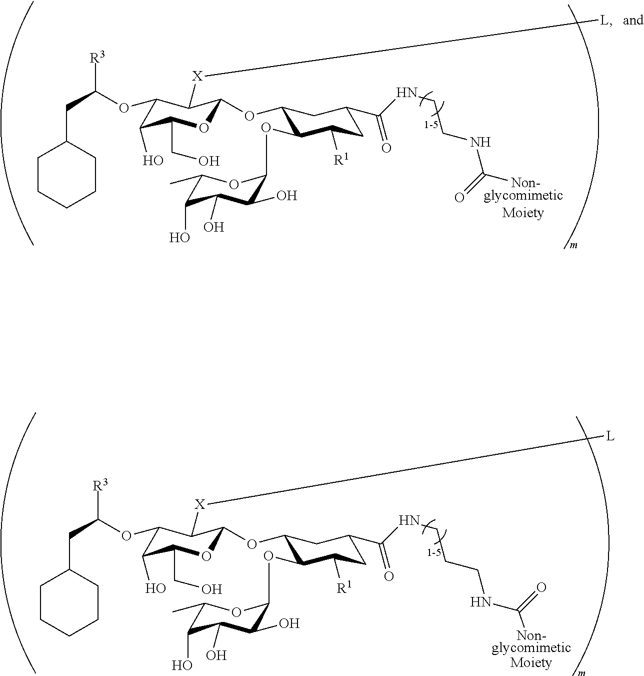

wherein each non-glycomimetic moiety, which may be identical or different, is independently chosen from R⁸, C₆₋₁₈ aryl-R⁸, and C₁₋₁₂ heteroaryl-R⁸ groups. In some embodiments, each non-glycomimetic moiety, which may be identical or different, is independently chosen from R⁸. In some embodiments, each non-glycomimetic moiety, which may be identical or different, is independently chosen from C₆₋₁₈ aryl-R⁸. In some embodiments, each non-glycomimetic moiety, which may be identical or different, is independently chosen from C₁₋₁₂ heteroaryl-R⁸ groups.

In some embodiments, at least one compound is chosen from compounds having the following Formula:

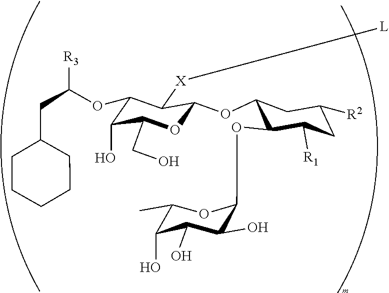

wherein each R², which may be identical or different, is independently chosen from a linker-non-glycomimetic moiety, wherein the non-glycomimetic moiety, which may be identical or different, is independently chosen from CXCR4 chemokine receptor inhibitors.

In some embodiments, at least one compound is chosen from compounds having the following Formulae:

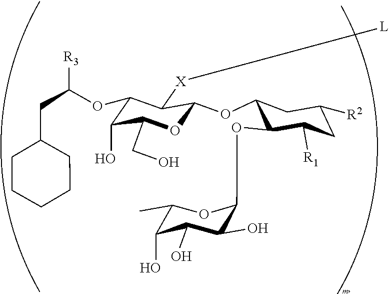

wherein each R², which may be identical or different, is independently chosen from a linker-non-glycomimetic moiety, wherein the non-glycomimetic moiety, which may be identical or different, is independently chosen from PEG groups.

In some embodiments, at least one compound is chosen from compounds having the following Formulae:

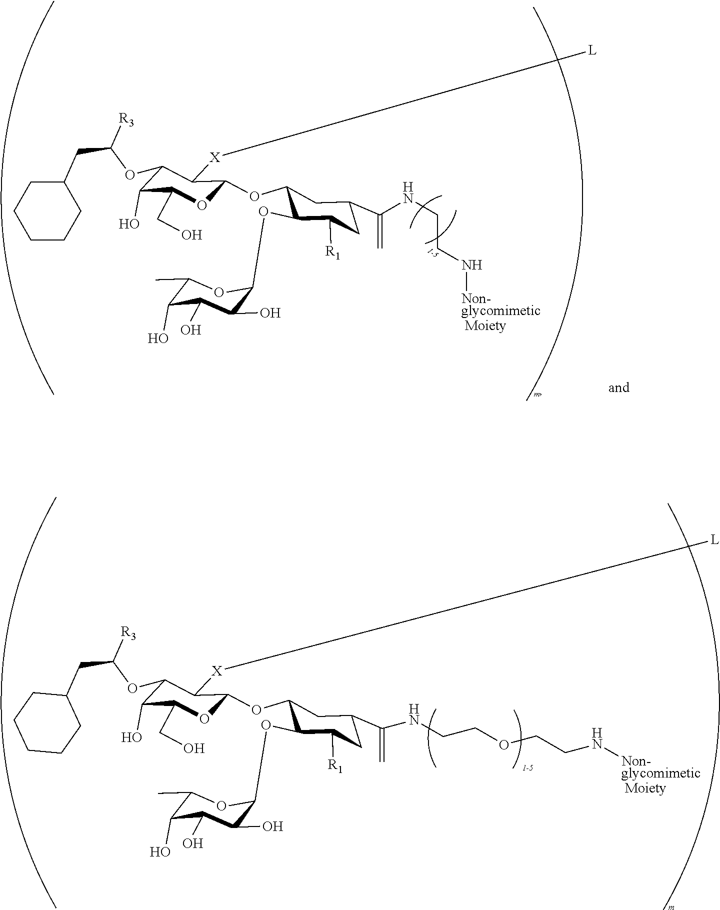

wherein each non-glycomimetic moiety, which may be identical or different, is independently chosen from PEG groups.

In some embodiments, at least one compound is chosen from compounds having the following Formulae:

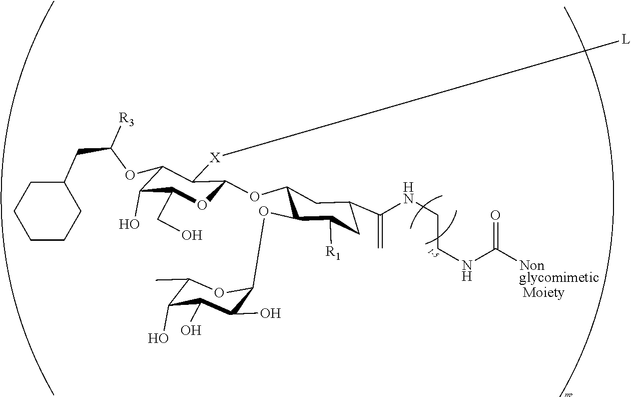

wherein each no-glycomimetic moiety, which may be identical or different, is independently chosen from PEG groups.

In some embodiments, at least one compound is chosen from compounds having the following Formula:

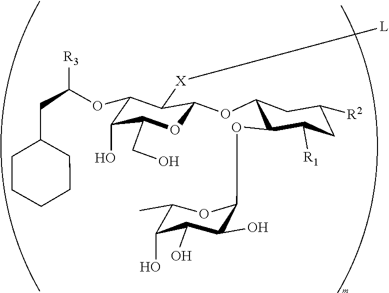

wherein each R³, which may be identical or different, is independently chosen from

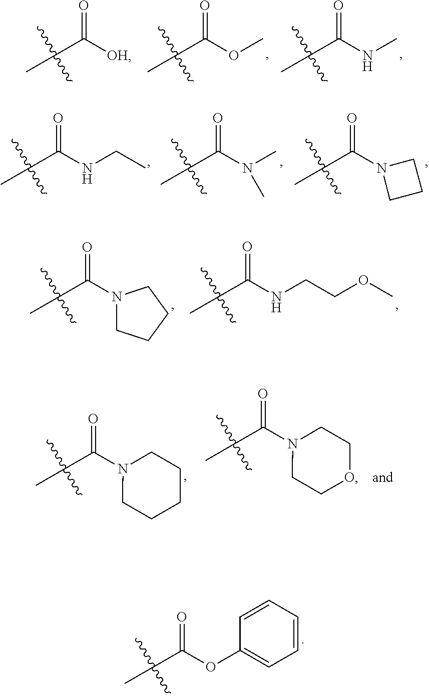

In some embodiments, at least one compound is chosen from compounds having the following Formula:

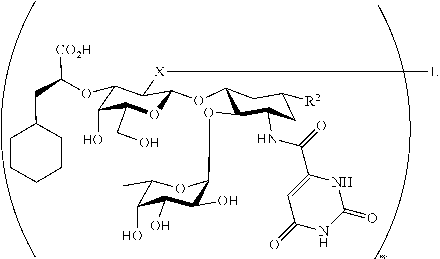

In some embodiments, at least one compound is chosen from compounds having the following Formula:

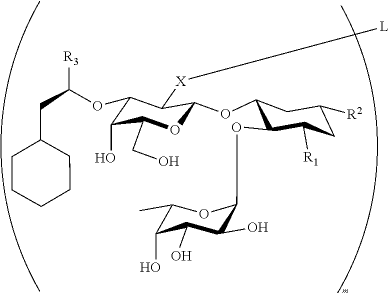

wherein each X, which may be identical or different, is independently chosen from —O— and —NH—.

In some embodiments, at least one compound is chosen from compounds having the following Formulae:

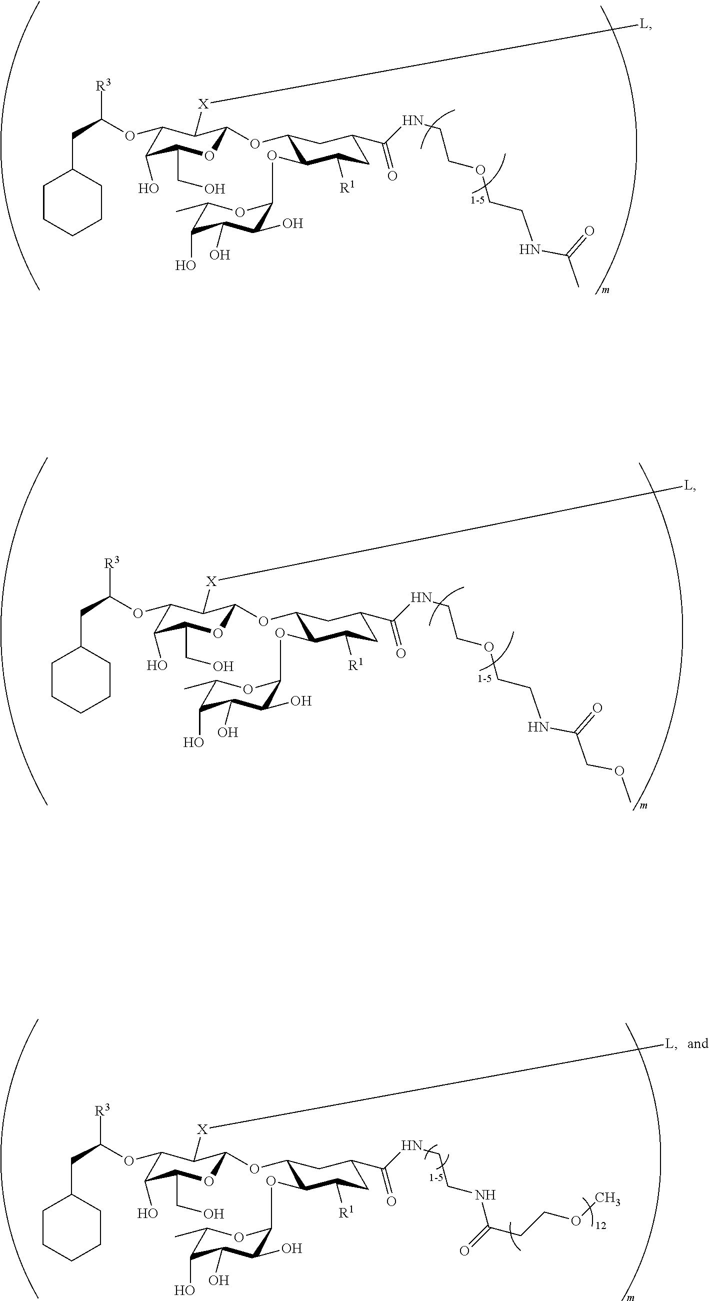

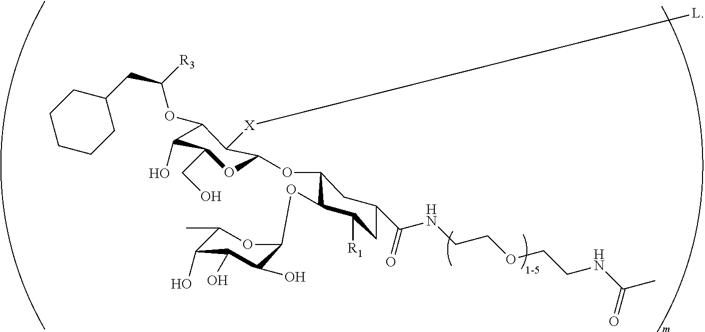

In some embodiments, at least one compound is chosen from compounds having the following Formulae:

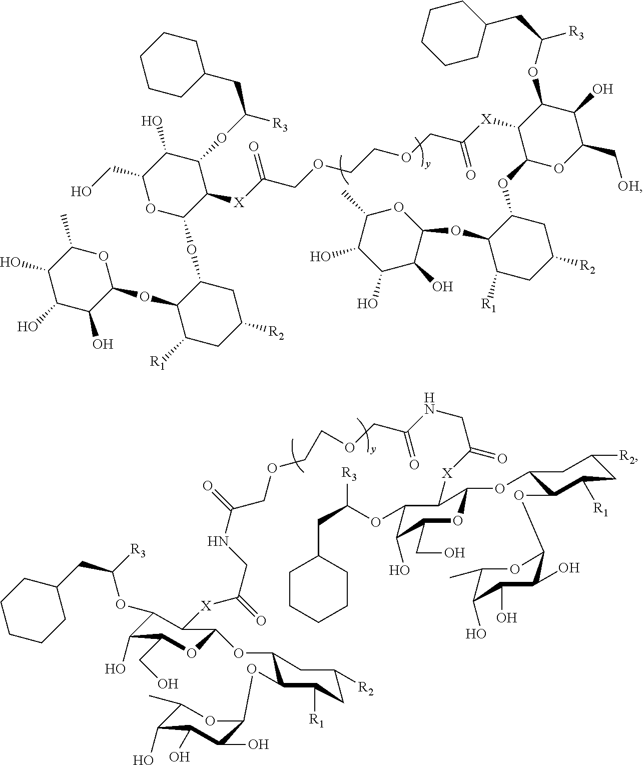

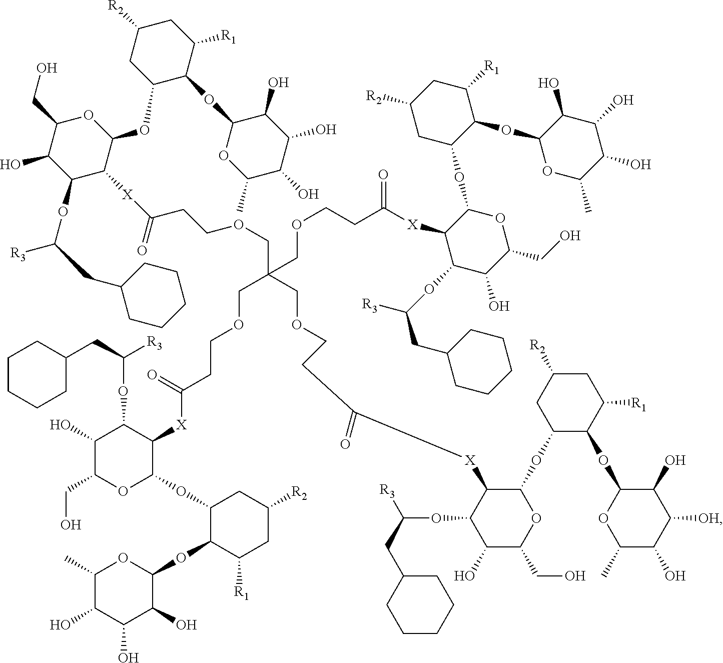

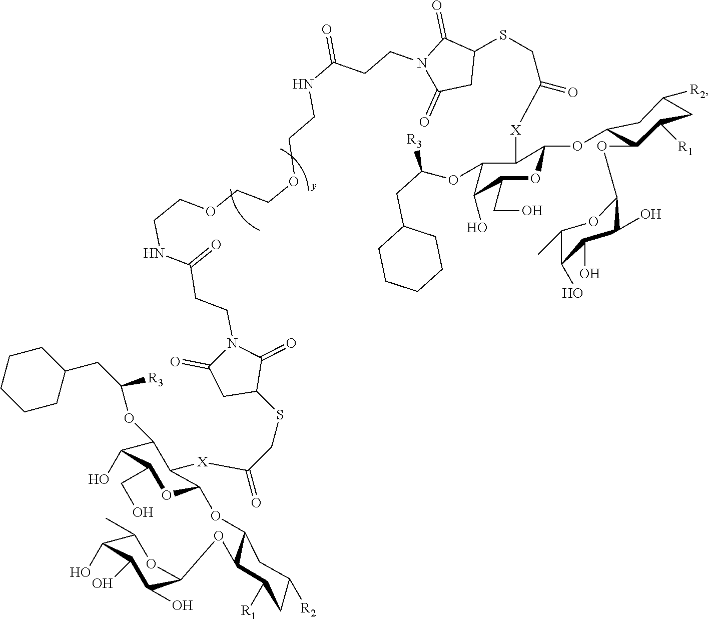

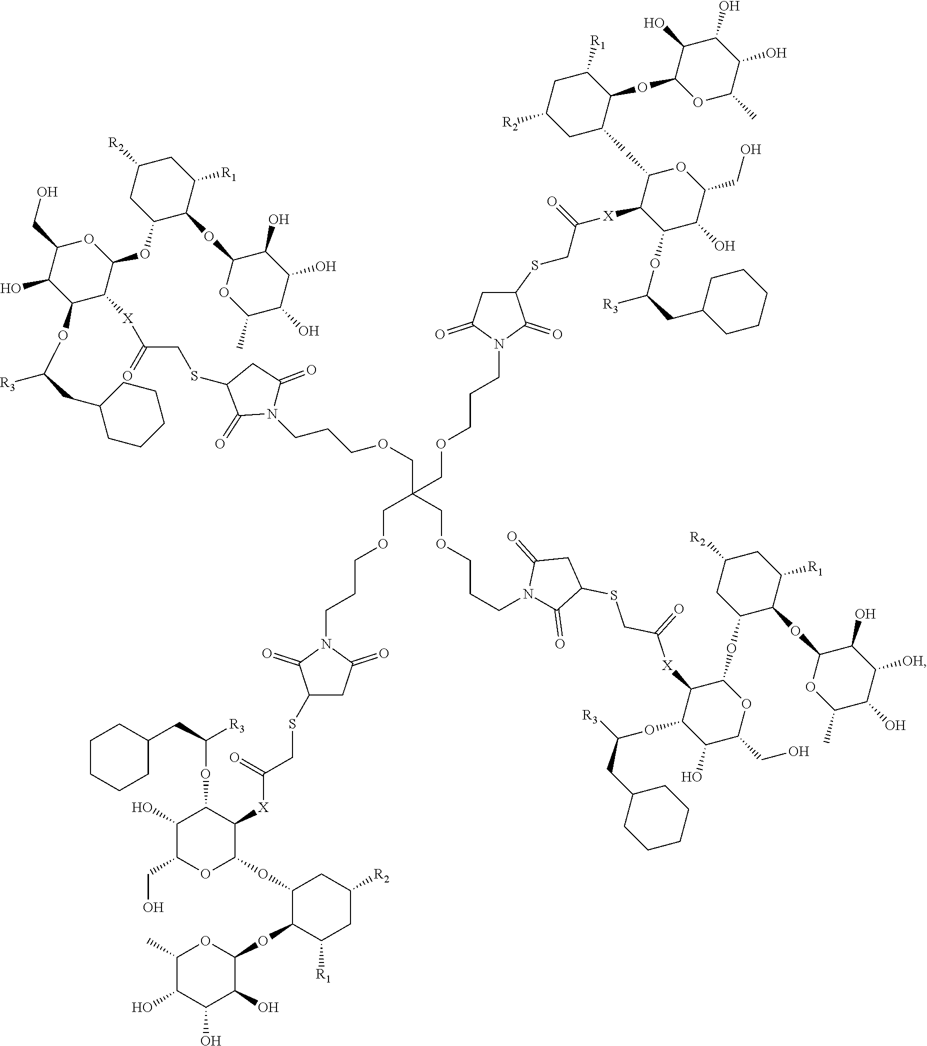

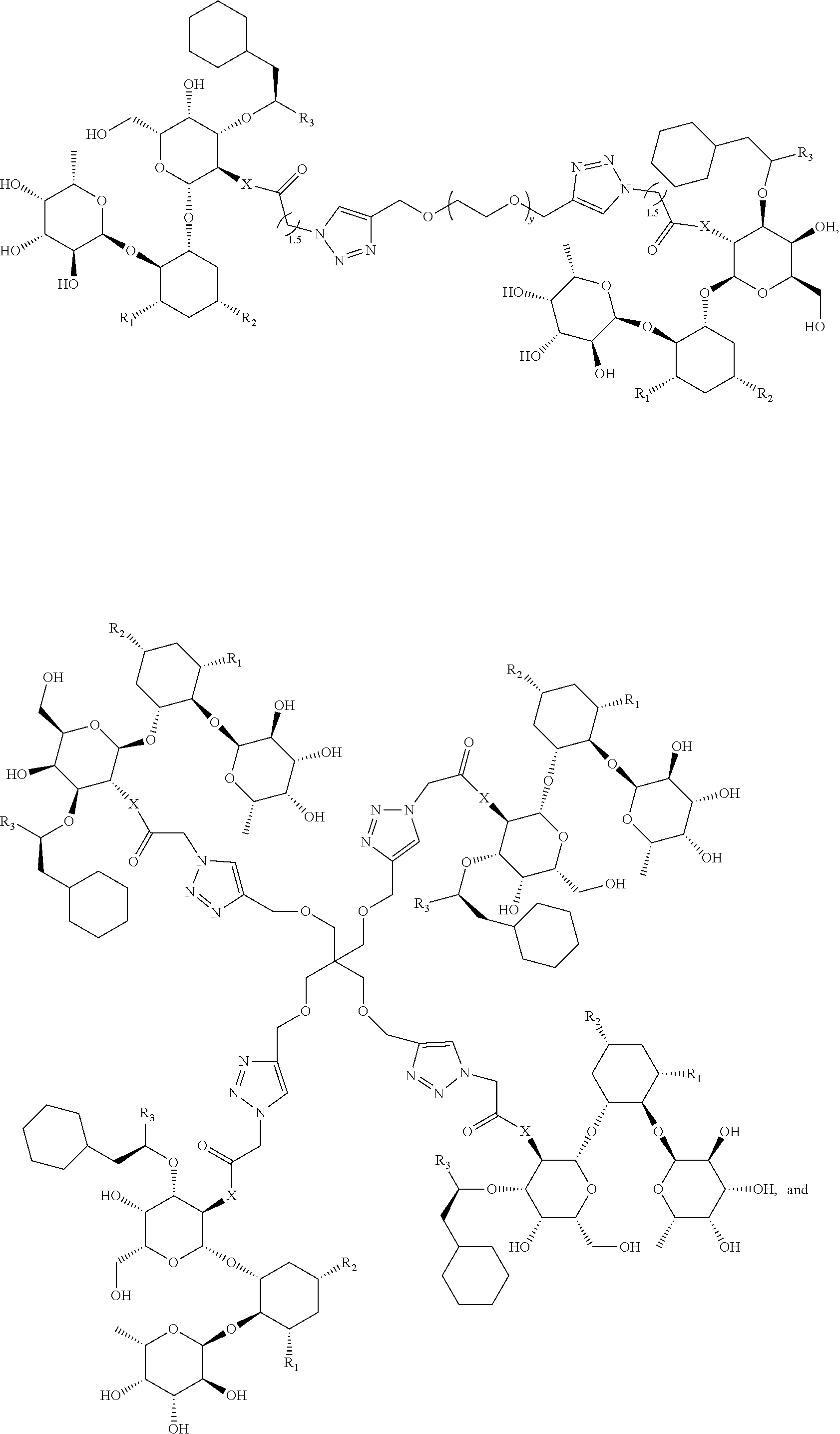

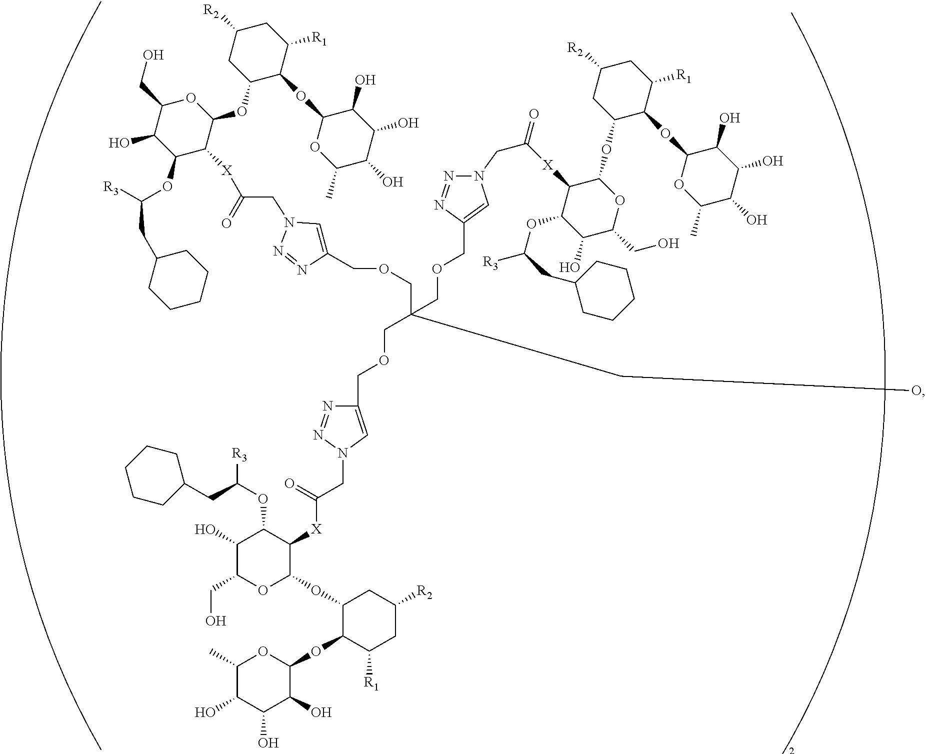

wherein y is chosen from integers ranging from 0 to 250.

In some embodiments, at least one compound is chosen from compounds having the following Formulae:

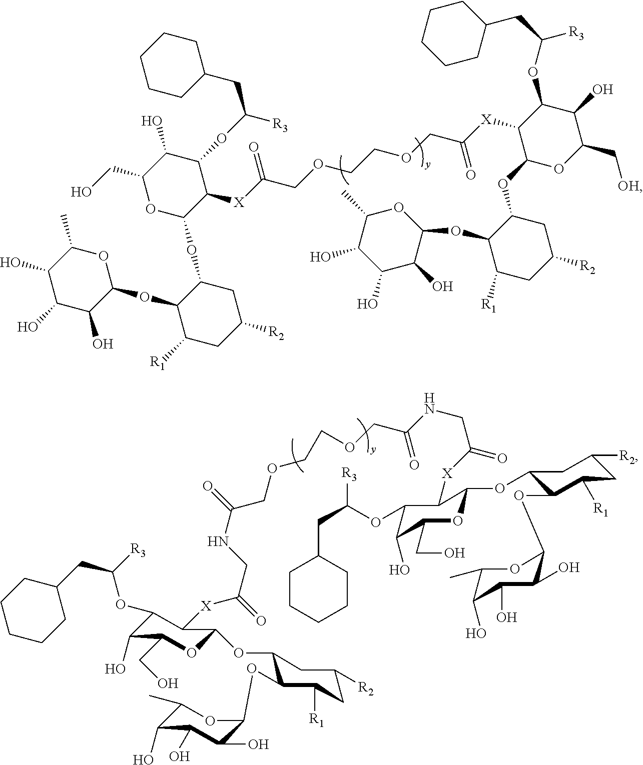

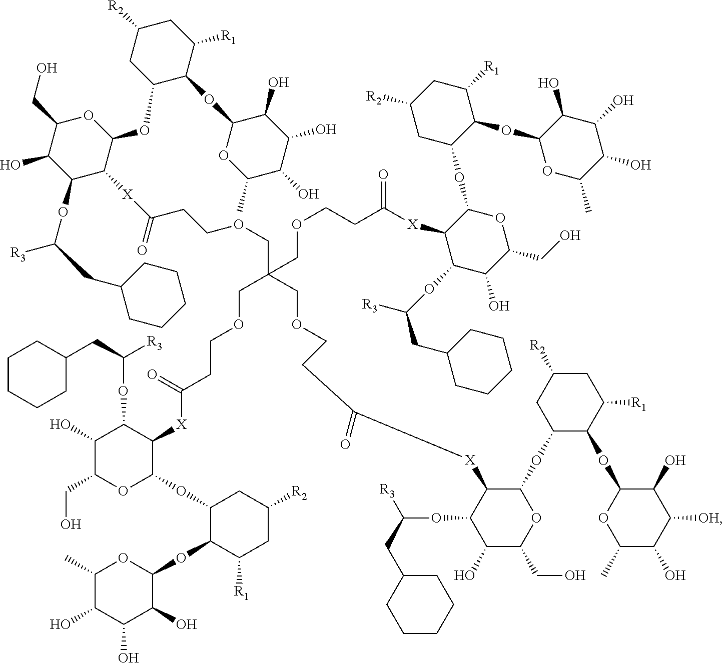

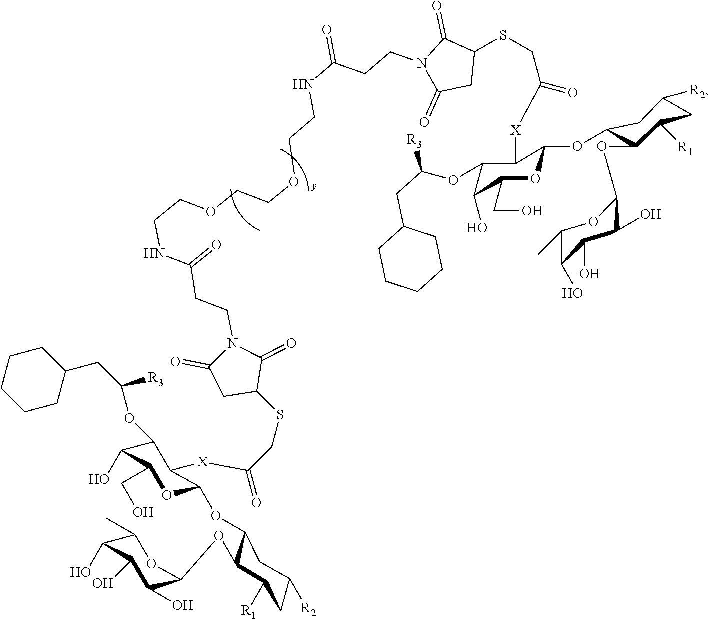

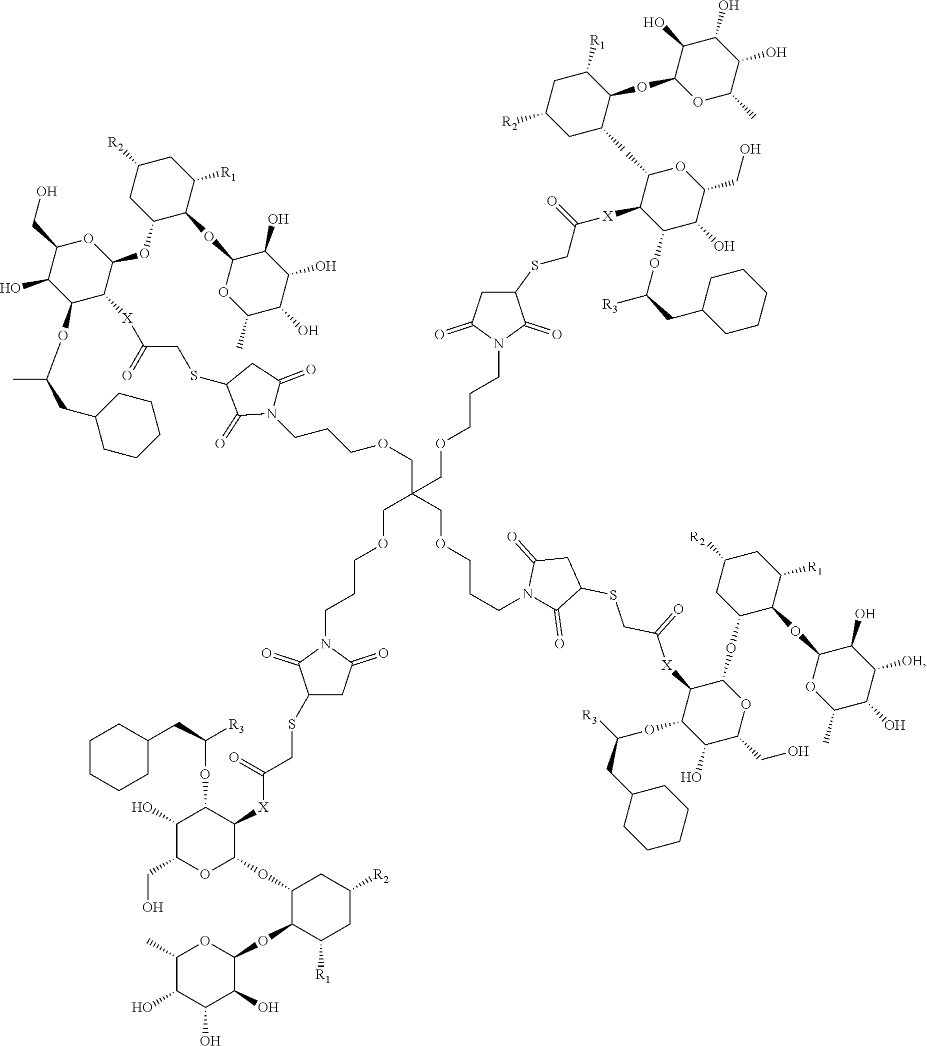

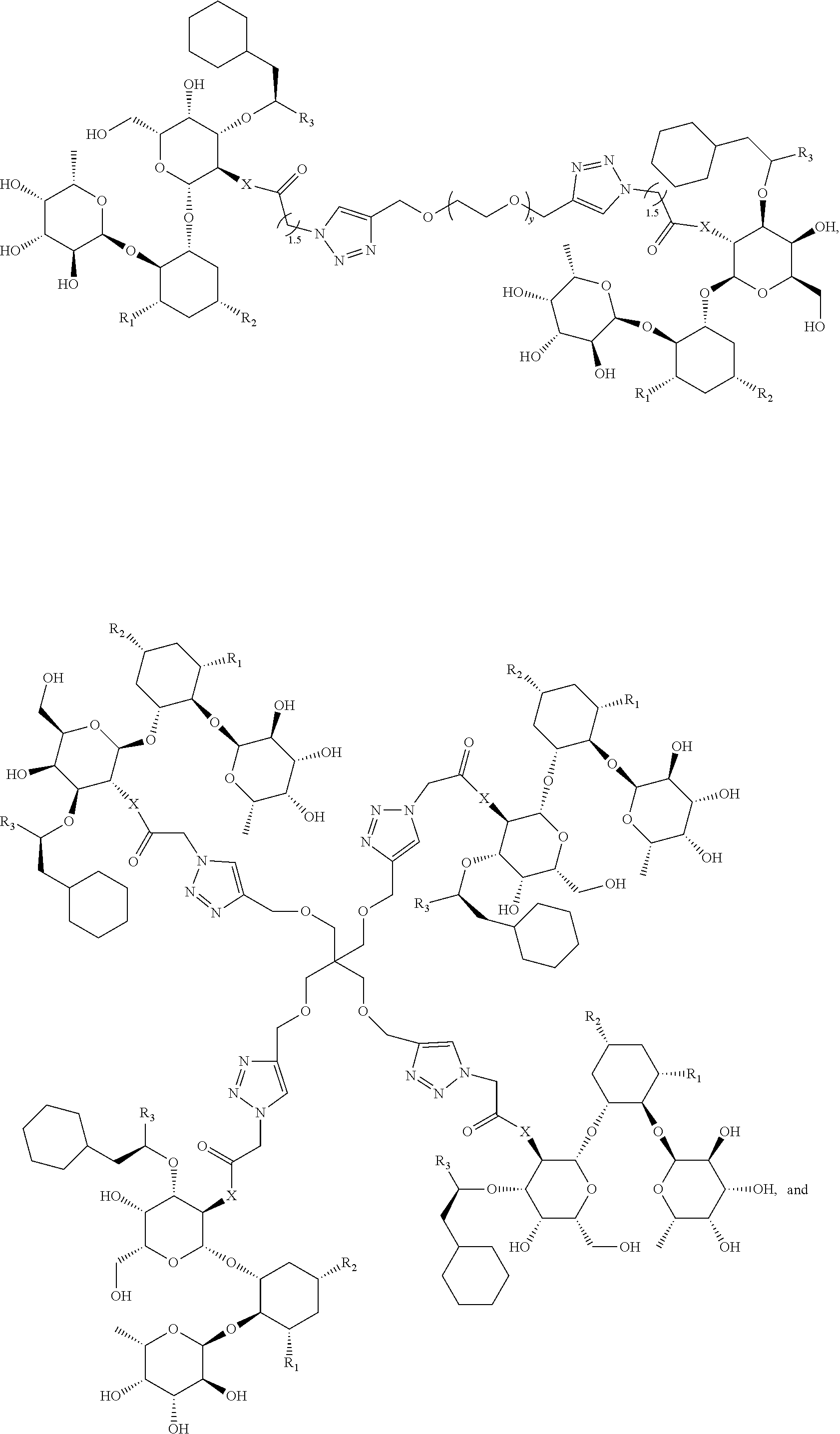

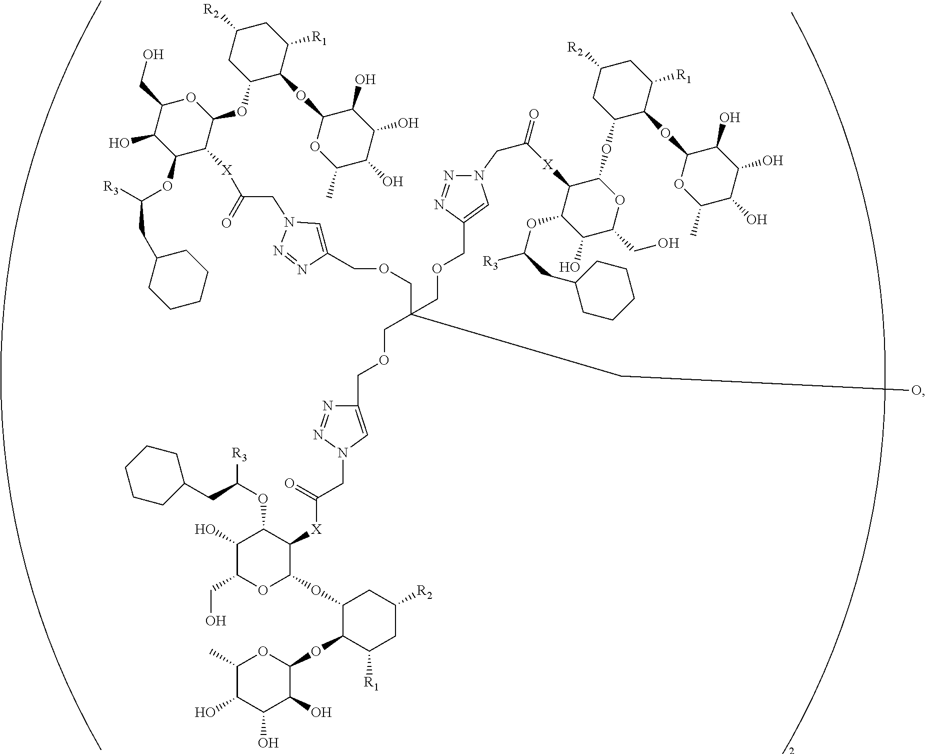

wherein each R¹, which may be identical or different, is independently chosen from methyl, ethyl, and

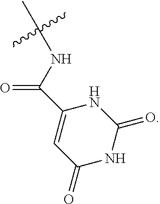 [SMILES: *NC(=O)c1cc(=O)[nH]c(=O)[nH]1]

In some embodiments, at least one compound is chosen from compounds having the following Formulae:

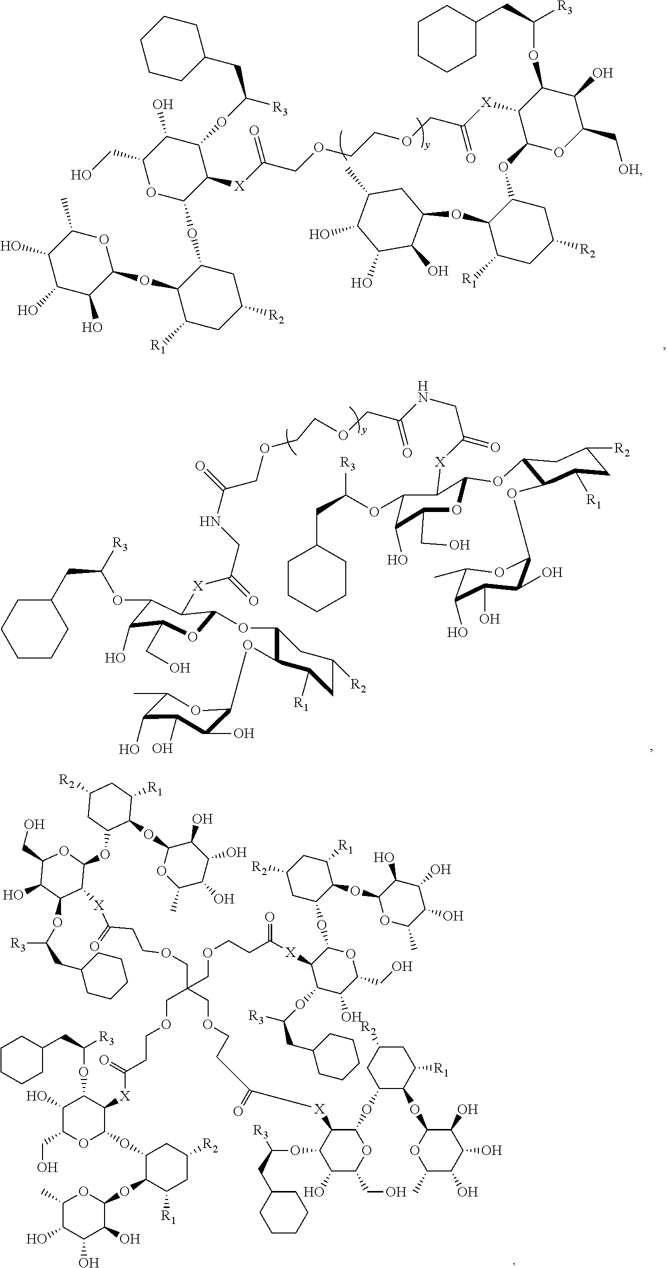

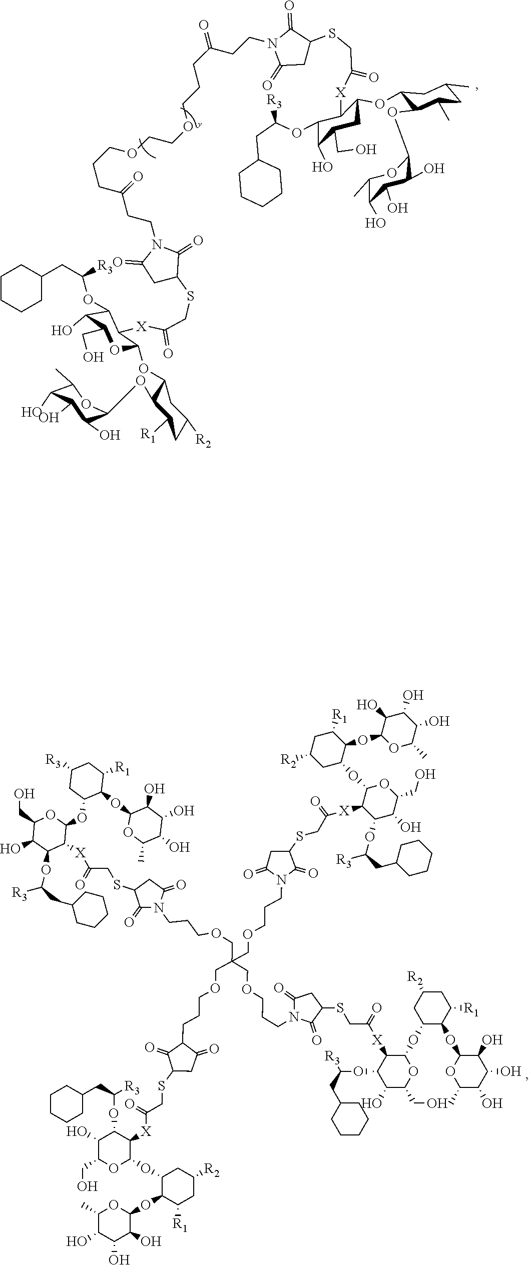

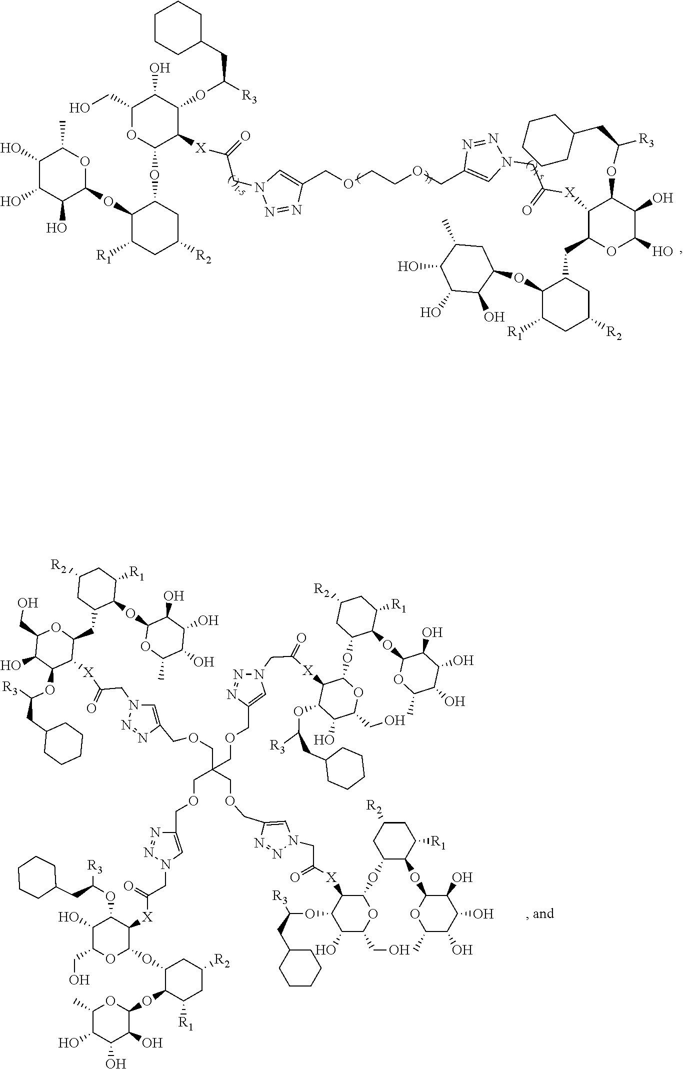

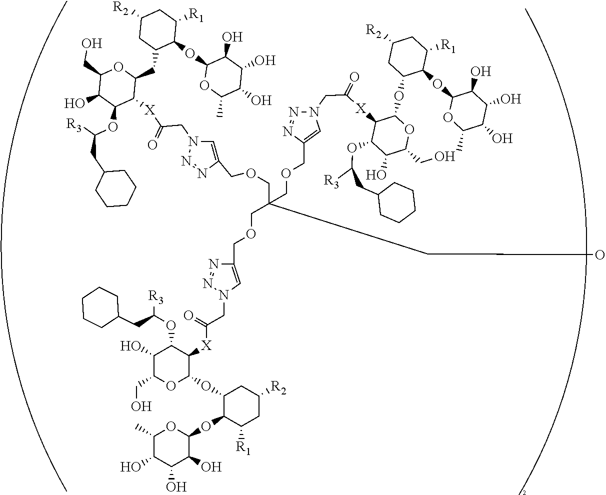

wherein each R₂, which may be identical or different, is independently chosen from a non-glycomimetic moiety.

In some embodiments, at least one compound is chosen from compounds having the following Formulae:

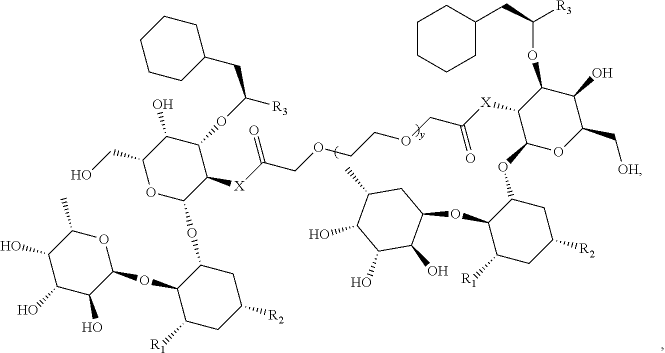

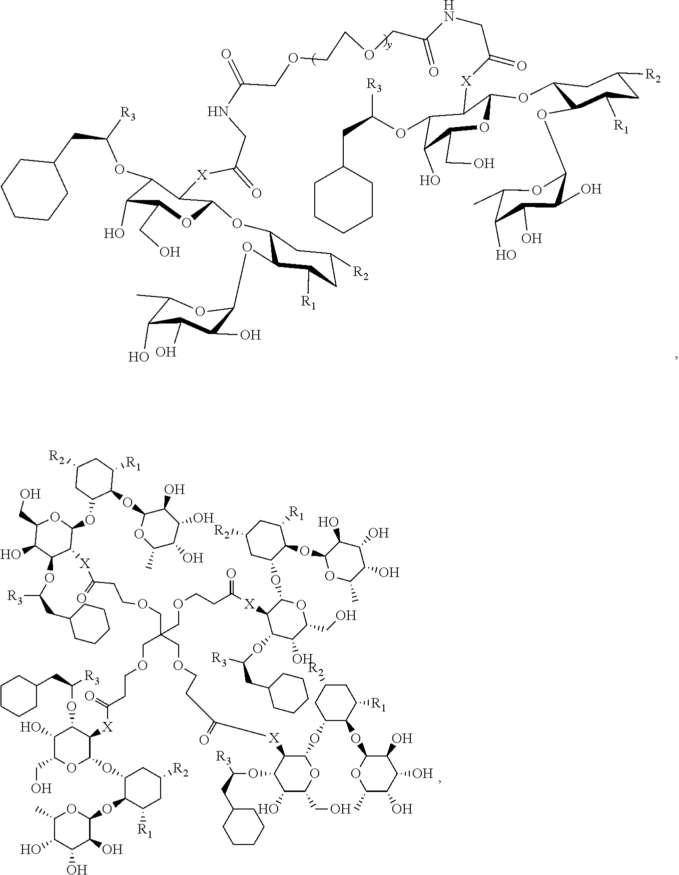

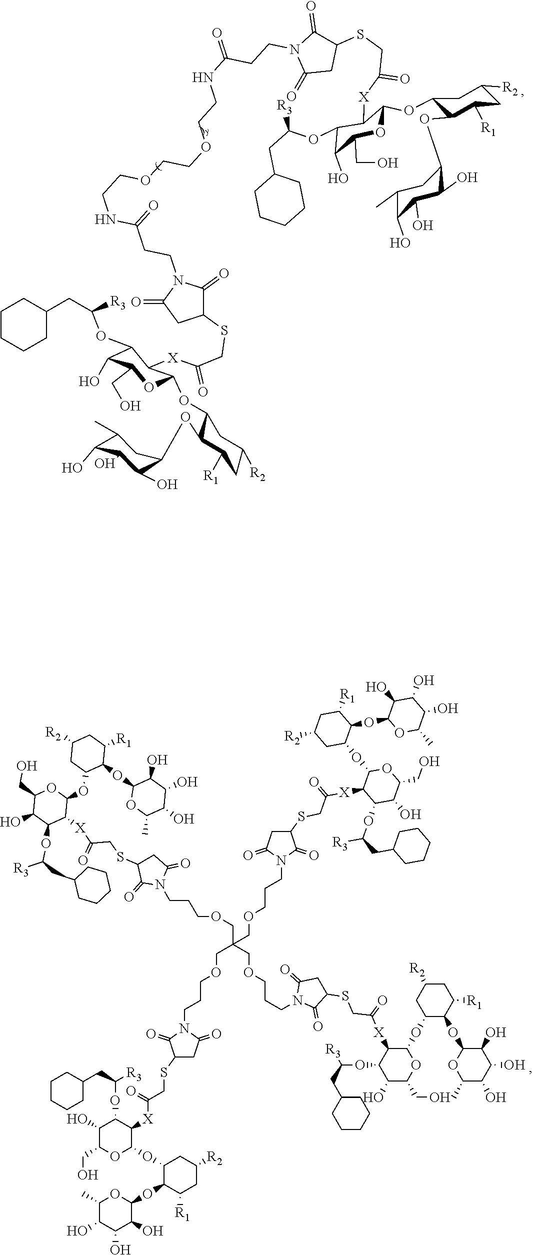

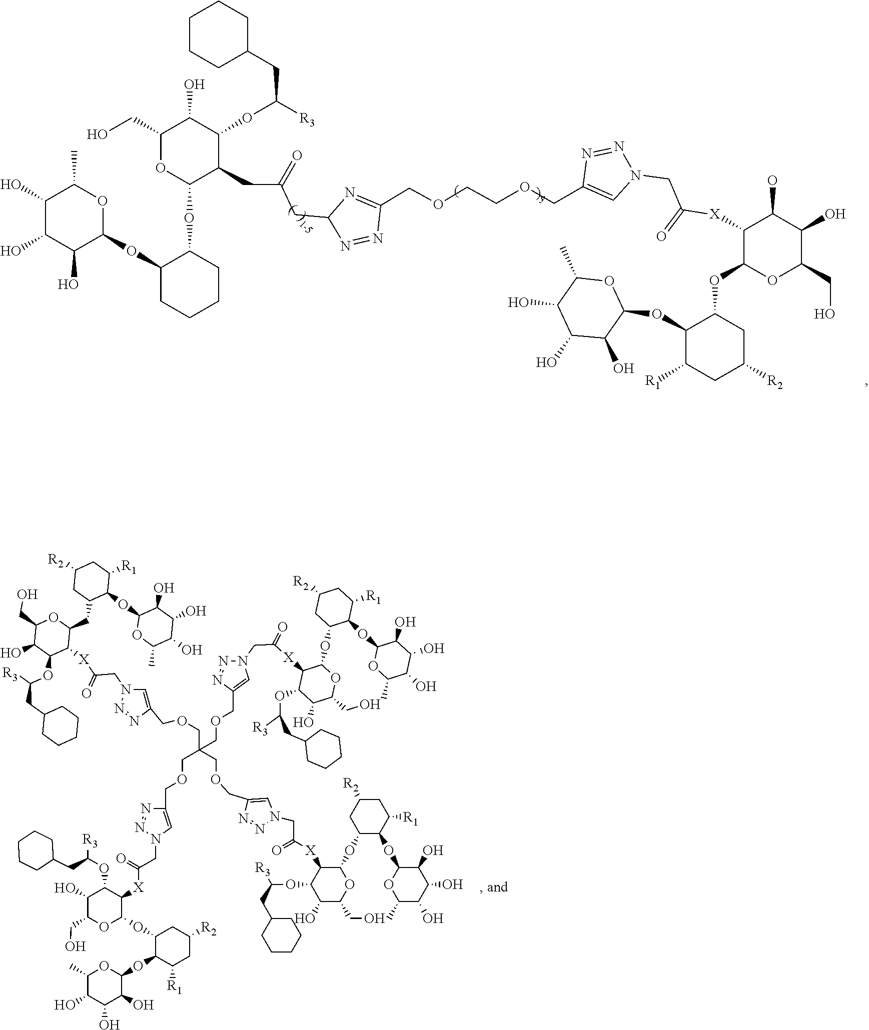

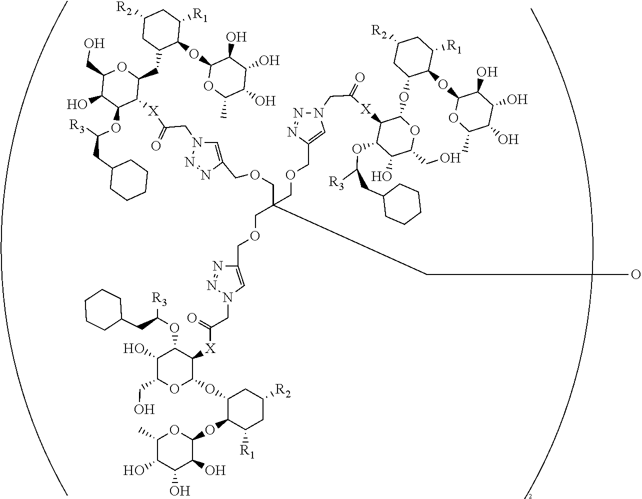

wherein each R₂, which may be identical or different, is independently chosen from a linker-non-glycomimetic moiety.

In some embodiments, at least one compound is chosen from compounds having the following Formulae:

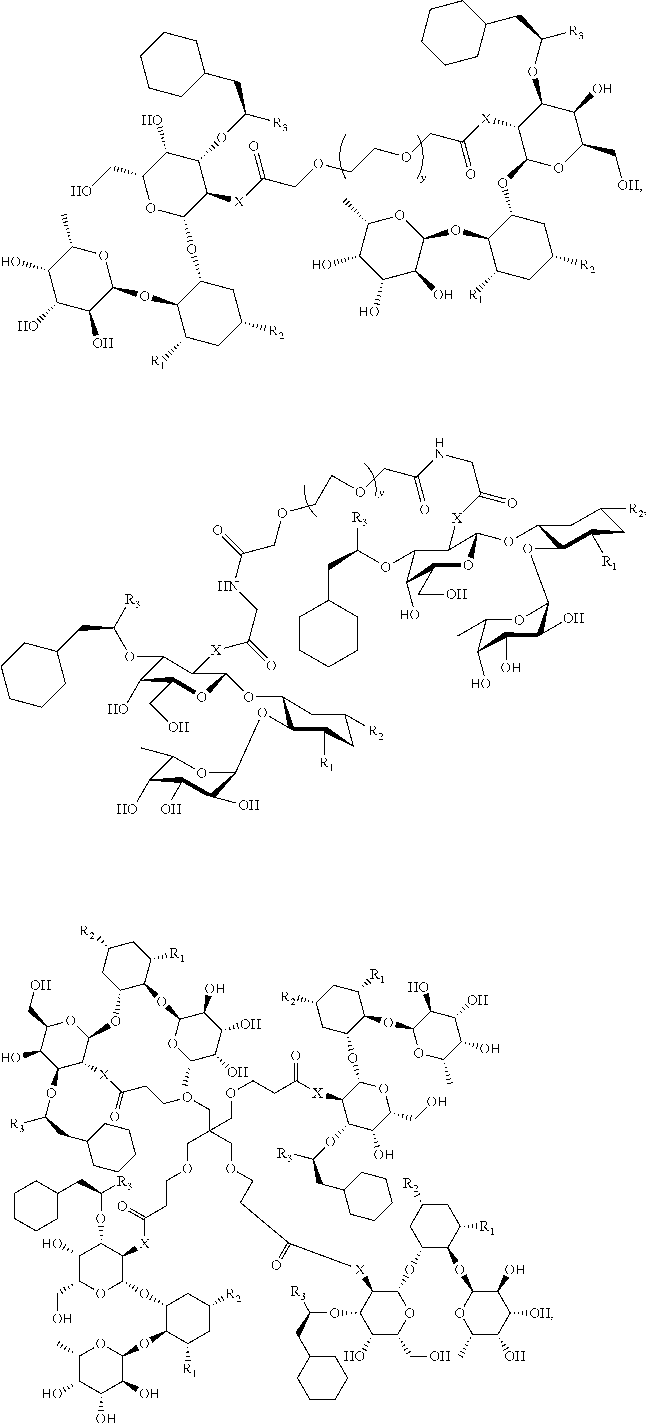

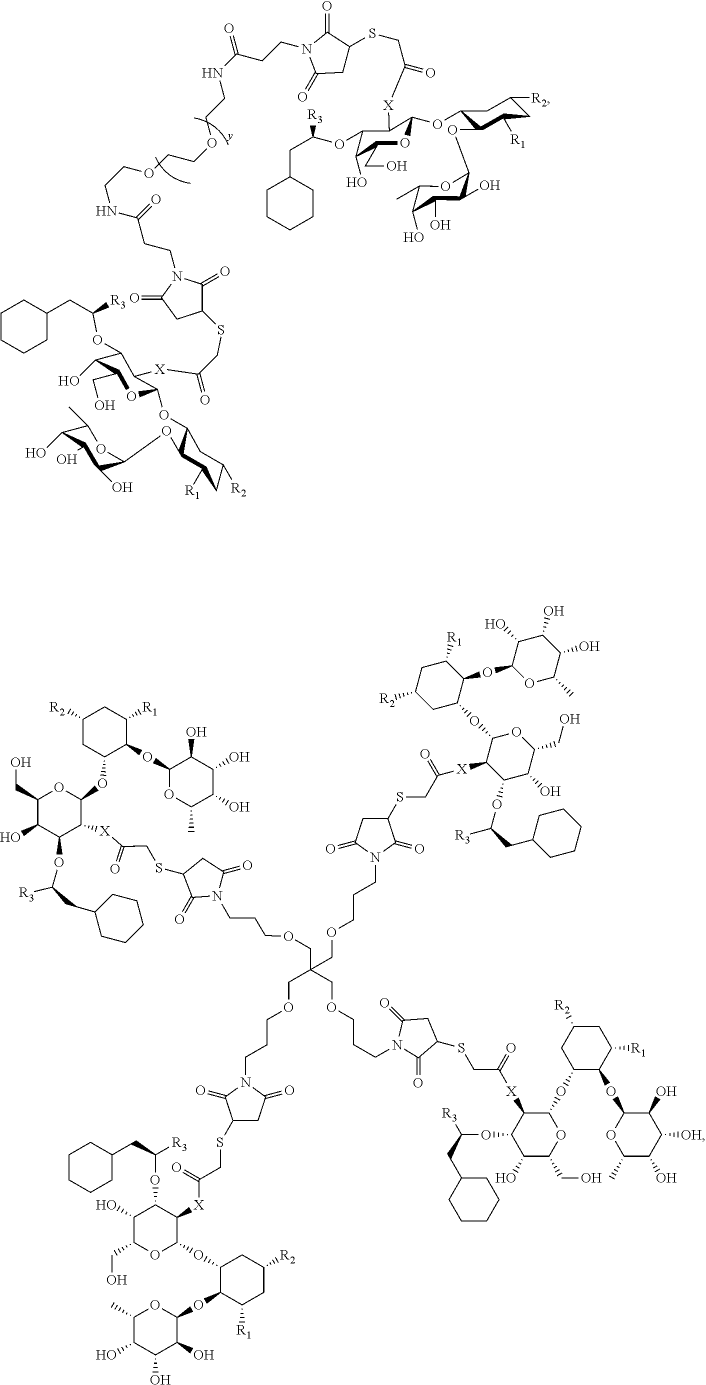

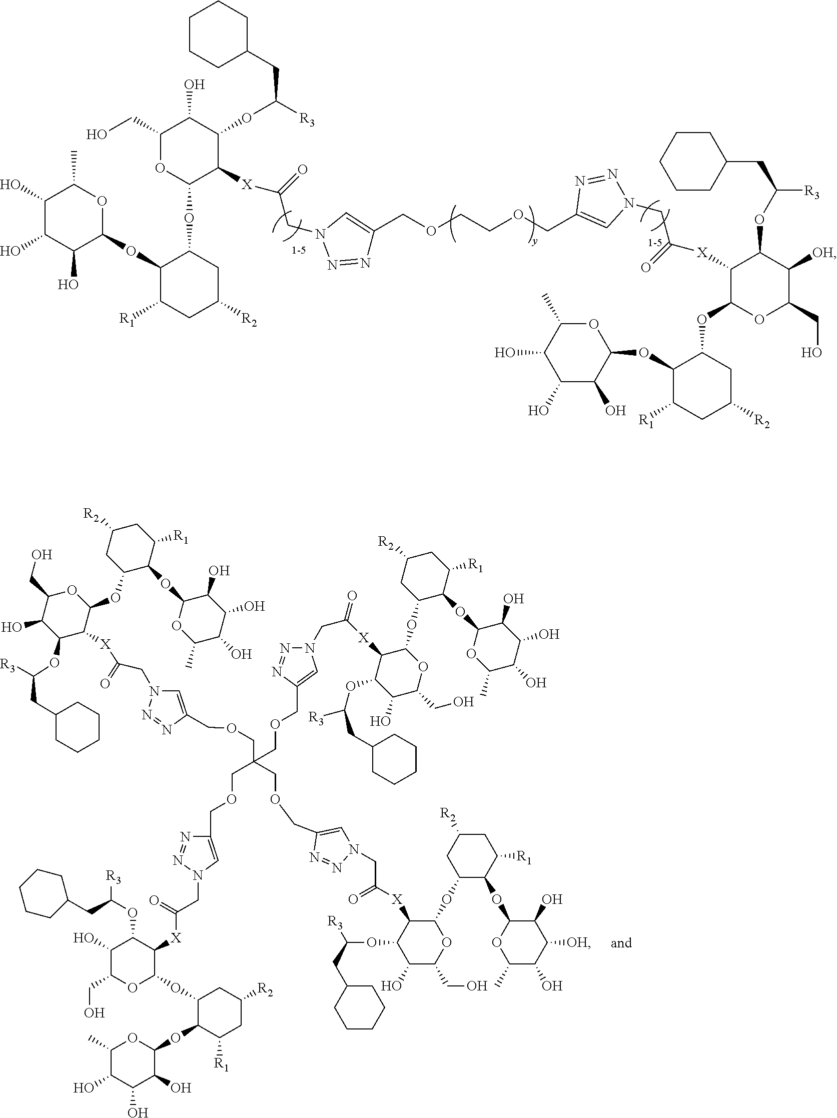

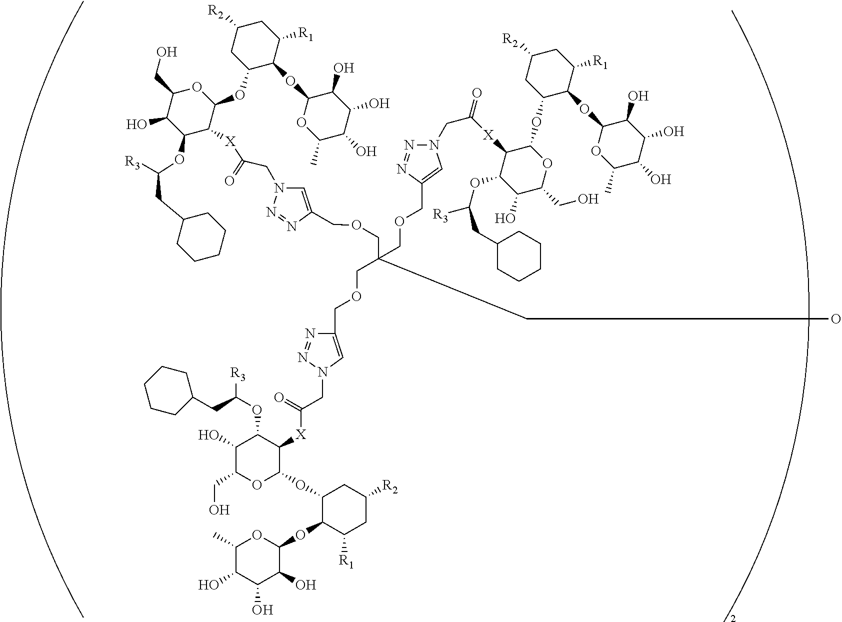

wherein each R², which may be identical or different, is independently chosen from H, a non-glycomimetic moiety, and a linker-non-glycomimetic moiety, wherein each non-glycomimetic moiety, which may be identical or different, is independently chosen from galectin-3 inhibitors, CXCR4 chemokine receptor inhibitors, polyethylene glycol, thiazolyl, chromenyl, C₁₋₈ alkyl, R⁸, C₆₋₁₈ aryl-R⁸, C₁₋₁₂ heteroaryl-R⁸,

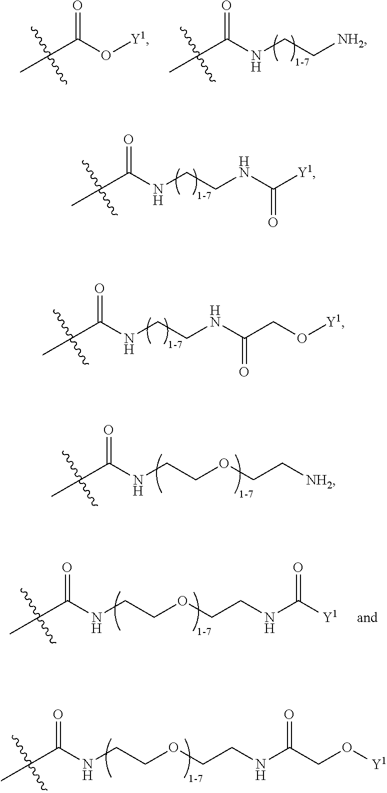

groups.

In some embodiments, at least one compound is chosen from compounds having the following Formulae:

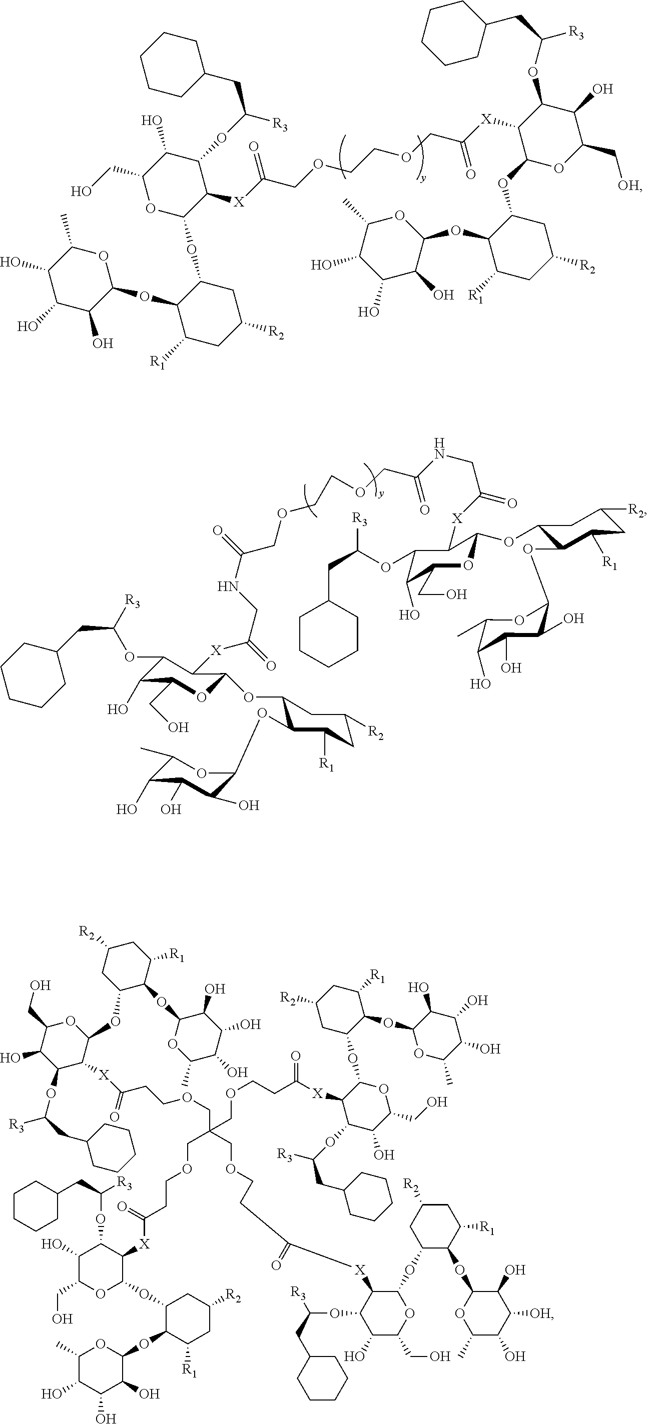

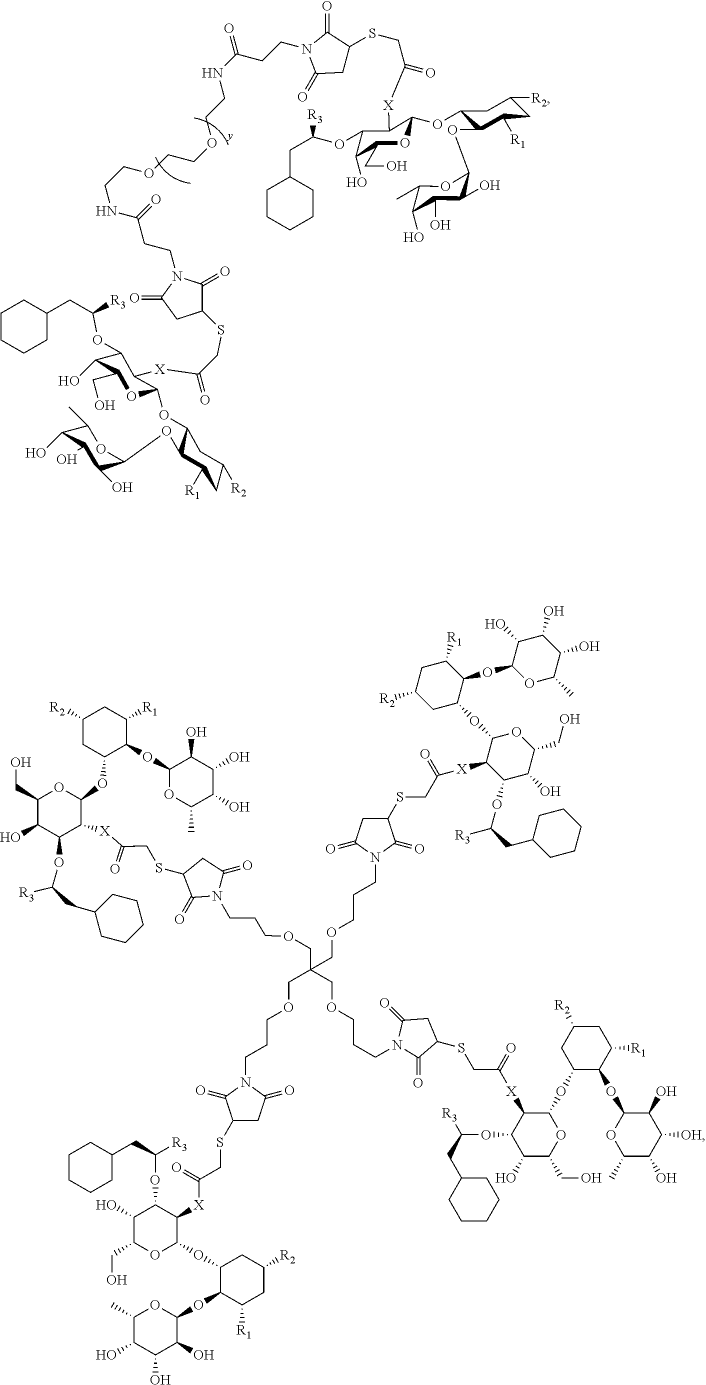

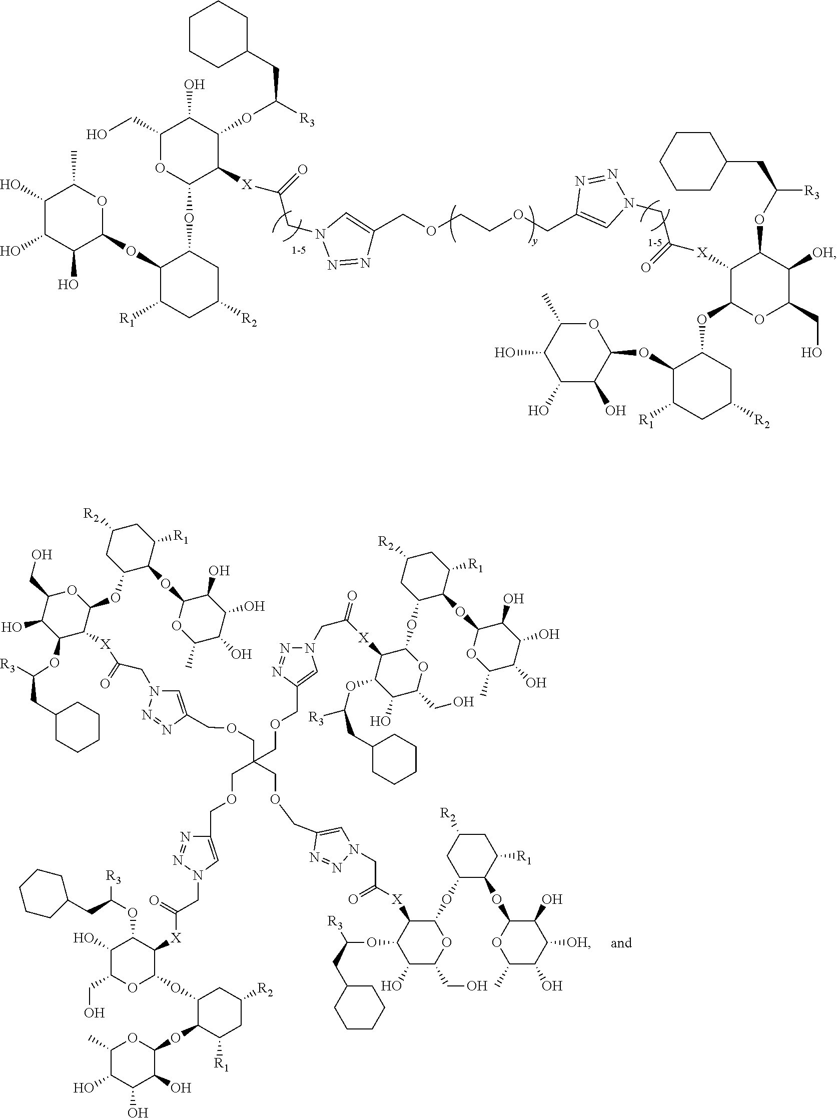

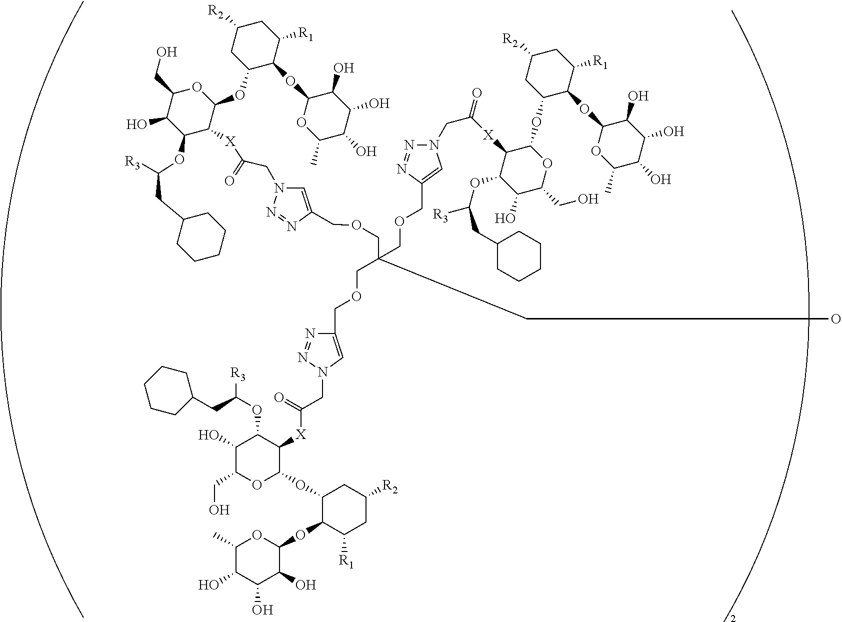

wherein each R³, which may be identical or different, is independently chosen from

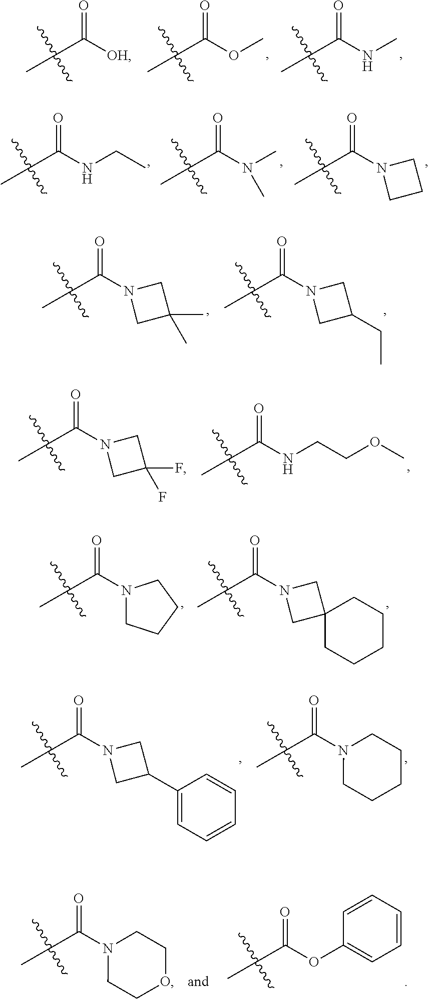

Also provided are pharmaceutical compositions comprising at least one compound of Formula (I). Such pharmaceutical compositions are described in greater detail herein. These compounds and compositions may be used in the methods described herein.

In some embodiments, a method for treating and/or preventing at least one disease, disorder, and/or condition where inhibition of E-selectin, galectin-3, and/or CXCR4 chemokine receptor mediated functions may be useful is disclosed, the method comprising administering at least one compound of Formula (I) and/or a pharmaceutical composition comprising at least one compound of Formula (I).

In some embodiments, a method for treating and/or preventing at least one inflammatory disease, disorder, and/or condition in which the adhesion and/or migration of cells occurs in the disease, disorder, and/or condition is disclosed, the method comprising administering at least one compound of Formula (I) and/or a pharmaceutical composition comprising at least one compound of Formula (I).

In some embodiments, a method for regulating the diffusion, compartmentalization, and/or endocytosis of plasma membrane glycoproteins and/or glycolipids is disclosed, the method comprising administering at least one compound of Formula (I) and/or a pharmaceutical composition comprising at least one compound of Formula (I).

In some embodiments, a method for regulating the selection, activation, and/or arrest of T cells, receptor kinase signaling, and/or the functionality of membrane receptors is disclosed, the method comprising administering at least one compound of Formula (I) and/or a pharmaceutical composition comprising at least one compound of Formula (I).

In some embodiments, a method for treating and/or preventing at least one fibrosis is disclosed, the method comprising administering at least one compound of Formula (I) and/or a pharmaceutical composition comprising at least one compound of Formula (I). In some embodiments, the at least one compound of Formula (I) inhibits lattice formation between galectin-3 and glycosylated ligands.

In some embodiments, a method for inhibiting adhesion of a cancer cell that expresses a ligand of E-selectin to an endothelial cell expressing E-selectin on the cell surface of the endothelial cell is disclosed, the method comprising contacting the endothelial cell and at least one compound of Formula (I) and/or a pharmaceutical composition comprising at least one compound of Formula (I) such that the at least one compound of Formula (I) interacts with E-selectin on the endothelial cell, thereby inhibiting binding of the cancer cell to the endothelial cell. In some embodiments, the endothelial cell is present in the bone marrow.

In some embodiment, a method for treating and/or preventing a cancer is disclosed, the method comprising administering to a subject in need thereof an effective amount of at least one compound of Formula (I) and/or a pharmaceutical composition comprising at least one compound of Formula (I). In some embodiments, at least one compound of Formula (I) and/or pharmaceutical composition comprising at least one compound of Formula (I) may be administered in conjunction with (i.e., as an adjunct therapy, which is also called adjunctive therapy) chemotherapy and/or radiotherapy.

The chemotherapy and/or radiotherapy may be referred to as the primary anti-tumor or anti-cancer therapy that is being administered to the subject to treat the particular cancer. In some embodiments, a method for reducing (i.e., inhibiting, diminishing) chemosensitivity and/or radiosensitivity of hematopoietic stem cells (HSC) to the chemotherapeutic drug(s) and/or radiotherapy, respectively, is disclosed, the method comprising administering to a subject in need thereof an effective amount of at least one compound of Formula (I) and/or a pharmaceutical composition comprising at least one compound of Formula (I).

In some embodiments, a method for enhancing (i.e., promoting) survival of hematopoietic stem cells is provided, the method comprising administering to a subject in need thereof at least one compound of Formula (I) and/or a pharmaceutical composition comprising at least one compound of Formula (I).

In some embodiments, a method for decreasing the likelihood of occurrence of metastasis of cancer cells (also called tumor cells herein) in a subject who is in need thereof is disclosed, the method comprising administering an effective amount of at least one compound of Formula (I) and/or a pharmaceutical composition comprising at least one compound of Formula (I).

In some embodiments, a method for treatment and/or prevention of at least one cancer in which the cancer cells may leave the primary site is disclosed, the method comprising administering to a subject in need thereof an effective amount of at least one compound of Formula (I) and/or a pharmaceutical composition comprising at least one compound of Formula (I). A primary site may be, for example, solid tissue (e.g., breast or prostate) or the bloodstream.

In addition to breast cancer, prostate cancer, and pancreatic cancer, other examples of infiltrating diseases include lung cancer and melanoma, as well as the hematological malignancies (e.g., leukemias and myelomas). As used herein, the term “treatment” (including variations such as “treating”) includes for the disease or a complication associated with the disease. For example, a complication associated with the cancer may not have presented itself in an individual with the disease, and a compound may be administered to prevent presentation of the complication in the individual. Complications associated with a cancer in which the cancer cells may leave the primary site include, for example, metastasis and infiltration of cancer cells to other tissues. For example, acute myelogenous leukemia (AML) and multiple myeloma (MM) cells migrate to the endosteal region of the bone marrow where the cells become quiescent and are protected from chemotherapy-induced apoptosis. Administration of a compound described herein may prevent adhesion or migration of cancer cells Such prevention can result in making the cancer cells more susceptible to treatment with chemotherapy. Administration of a compound described herein in the context of prevention may be to an individual who is at risk of occurrence of a cancer for the first time, or for recurrence of a cancer. For example, while a brain cancer such as glioblastoma multiforme is typically treated with another type of therapy (such as radiation or chemotherapy) for the first occurrence, such therapy is usually not effective to prevent recurrence.

In some embodiments, a method for treatment and/or prevention of at least one cancer in which it is desirable to mobilize cancer cells from a site into the bloodstream and/or retain the cancer cells in the bloodstream is disclosed, the method comprising administering to a subject in need thereof an effective amount of at least one compound of Formula (I) and/or a pharmaceutical composition comprising at least one compound of Formula (I).

One use of the method is, for example, for stem cell harvesting. Stem cells may be needed, for example, after high-dose chemotherapy treatment. Many chemotherapies suppress bone marrow which disrupts the production of certain components of blood in an individual. As a result, the individual may develop a variety of blood cell related disorders and continuation of chemotherapy may be compromised. A compound described herein may be used, for example, to release stem cells into circulating blood and enhance retention of the stem cells in the blood. The method may include a further step of collecting cells that are released. For example, released stem cells may be collected. A variety of techniques are known in the art for collecting cells. For example, apheresis may be utilized. An example of a stem cells is a bone marrow progenitor cell. The release of such cells from bone marrow into circulating blood and retention therein has a variety of uses. For example, the mobilized bone marrow progenitor cells may be collected from the blood. A use of such collected cells is to obtain healthy bone marrow progenitor cells from an individual prior to treatment of the individual in a manner such that bone marrow is suppressed. Following treatment, the individual can receive a bone marrow transplantation utilizing the bone marrow progenitor cells collected prior to treatment. This is useful, for example, where an individual needs to be subjected to a chemotherapy protocol that will suppress bone marrow.

It can be desirable to additionally treat an individual with at least one (i.e., one or more) colony stimulating factor. Such a factor may be administered, for example, before or simultaneous with administration of at least one of the above-described compounds. Where administration is simultaneous, the combination may be administered from a single container or two (or more) separate containers. An example of a suitable colony stimulating factor is granulocyte-colony stimulating factor (G-CSF). G-CSF induces the bone marrow to grow and produce more stem cells. A compound described herein aids in releasing stem cells into circulating blood. Stem cells produced in bone marrow and released into circulating blood, as a result of the combination of the administration (separately or together) of a compound described herein and G-CSF, may be collected as described above. Such collected stem cells may be, for example, administered to the individual after chemotherapy. The stem cells return to the bone marrow and produce blood cells. Application of a compound described herein to mobilization and harvesting of healthy bone marrow progenitor cells from bone marrow treated with G-CSF provides cells useful, for example, for bone marrow transplantation.

In some embodiments, at least one compound of Formula (I) and/or a pharmaceutical composition comprising at least one compound of Formula (I) may be used in methods described herein for treatment and/or prevention of tumor metastasis. In some embodiments, the tumor metastasis arises from pancreatic cancer. In some embodiments, the tumor metastasis arises from prostate cancer. In some embodiments, the tumor metastasis arises from pancreatic cancer. In some embodiments, the tumor metastasis arises from breast cancer. In some embodiments, at least one additional chemotherapy agent such as gemcitabine is administered to the individual.

In some embodiments, a method for decreasing the likelihood of occurrence of infiltration of cancer cells into bone marrow is disclosed, the method comprises administering to a subject in need thereof an effective amount of at least one compound of Formula (I) and/or a pharmaceutical composition comprising at least one compound of Formula (I).

In some embodiments, a method for releasing cells into circulating blood and enhancing retention of the cells in the blood is disclosed, the method comprising administering to a subject in need thereof an effective amount of at least one compound of Formula (I) and/or a pharmaceutical composition comprising at least one compound of Formula (I). In some embodiments, the method further includes collecting the released cells. In some embodiments, collecting the released cells utilizes apheresis. In some embodiments, the released cells are stem cells (e.g., bone marrow progenitor cells). In some embodiments, G-CSF is administered to the individual.

In some embodiments, a method for treating and/or preventing thrombosis is disclosed, the method comprising administering to a subject in need thereof an effective amount of at least one compound of Formula (I) and/or a pharmaceutical composition comprising at least one compound of Formula (I).

In some embodiments, a method for treating and/or preventing mucositis is disclosed, the method comprising administering to a subject in need thereof an effective amount of at least one compound of Formula (I) and/or a pharmaceutical composition comprising at least one compound of Formula (I).

In some embodiments, a method for treating and/or preventing one cardiovascular disease, disorder and/or condition is disclosed, the method comprising administering to a subject in need thereof an effective amount of at least one compound of Formula (I) and/or a pharmaceutical composition comprising at least one compound of Formula (I).

In some embodiments, a method for treatment and/or prevention of atherosclerosis is disclosed, the method comprising administering to a subject in need thereof an effective amount of at least one compound of Formula (I) and/or a pharmaceutical composition comprising at least one compound of Formula (I).

In some embodiments, a method for inhibiting the rejection of transplanted tissue is disclosed, the method comprising administering to a subject in need thereof an effective amount of at least one compound of Formula (I) and/or a pharmaceutical composition comprising at least one compound of Formula (I).

In some embodiments, a method for treatment and/or prevention of pathological angiogenesis is disclosed, the method comprising administering to a subject in need thereof an effective amount of at least one compound of Formula (I) and/or a pharmaceutical composition comprising at least one compound of Formula (I).

In some embodiments, a method for treatment and/or prevention of an epileptic syndrome is disclosed, the method comprising administering to a subject in need thereof at least one compound of Formula (I) and/or a pharmaceutical composition comprising at least one compound of Formula (I). Examples of an epileptic syndrome include epilepsy, Rasmussen's syndrome and West syndrome. Other syndromes which are multi-system disorders but with the primary disability resulting from neurological effects including epilepsy, are considered epileptic syndromes for purposes of the present invention. An example of such a syndrome is tuberous sclerosis syndrome.

In some embodiments, a method for treatment and/or prevention of a neurodegenerative disease is disclosed, the method comprising administering to a subject in need thereof an effective amount of at least one compound of Formula (I) and/or a pharmaceutical composition comprising at least one compound of Formula (I). Examples of neurodegenerative diseases include such as selected from Parkinson's disease, dementia with Lewy bodies, pure autonomic failure (PAF), Alzheimer's disease, neurodegeneration with brain iron accumulation, type I (also referred to as adult neuroaxonal dystrophy or Hallervorden-Spatz syndrome), traumatic brain injury, amyotrophic lateral sclerosis, Pick disease, multiple system atrophy (including Shy-Drager syndrome, striatonigral degeneration, and olivopontocerebellar atrophy) and stroke, multiple sclerosis, epilepsy and infantile neuroaxonal dystrophy.

In some embodiments, a method for treatment and/or prevention of α-synucleinopathies is disclosed, the method comprising administering to a subject in need thereof an effective amount of at least one compound of Formula (I) and/or a pharmaceutical composition comprising at least one compound of Formula (I).

The compounds of the present disclosure and pharmaceutical compositions comprising at least one such compound may be administered in combination with at least one additional agent for the treatment of neurodegeneration or symptoms thereof (e.g. donepezil, galantamine, memantine, rivastigmine, levodopa, carbidopa, dopamine agonists, COMT inhibitors, MAO inhibitors, anticholinergic agents, corticosteroids, beta interferons, ocrelizumab, glatiramer acetate, dimethyl fumarate, fingolimod, teriflunomide, natalizumab, alemtuzumab, mitoxantrone, riluzole, edaravone). The compounds of the present disclosure and pharmaceutical composition comprising at least one such compound may be administered before, after, or concurrently with administration of at least one additional agent for the treatment of neurodegeneration or symptoms thereof. Where administration is concurrent, the combination may be administered from a single container or two (or more) separate containers.

In some embodiments, a method for treatment and prevention of a fibrosing disease or condition is disclosed, the method comprising administering to a subject in need thereof an effective amount of at least one compound of Formula (I) and/or a pharmaceutical composition comprising at least one compound of Formula (I). Examples of fibrosing diseases and conditions include such as selected from rheumatoid arthritis, lupus, pathogenic fibrosis, fibrosing disease, heart disease, heart remodeling post MI, nonalcoholic fatty liver disease (NASH), idiopathic pulmonary fibrosis (IPF), fibrosis associated with thrombosis, fibrosis associated with macular degeneration, fibrotic lesions such as those formed after Schistosoma japonicum infection, radiation damage, autoimmune diseases, Lyme disease, chemotherapy induced fibrosis, HIV or infection-induced focal Sclerosis, failed back syndrome due to spinal Surgery scarring, abdominal adhesion post-Surgery scarring, fibrocystic formations, fibrosis after spinal injury, Surgery-induced fibrosis, mucosal fibrosis, peritoneal fibrosis caused by dialysis, Adalimumab-associated pulmonary fibrosis, and nephrogenic fibrosing dermopathy.

In some embodiments, the fibrosis is fibrosis of the liver resulting from conditions including but not limited to alcohol, drug, or chemically induced cirrhosis, ischemia-reperfusion injury after hepatic transplant, necrotizing hepatitis, hepatitis B, hepatitis C, primary biliary cirrhosis, primary sclerosing cholangitis, and nonalcoholic steatohepatitis.

In some embodiments, the fibrosis is fibrosis in the kidney resulting from conditions including but not limited to proliferative and Sclerosing glomerulonephritis, nephrogenic fibrosing dermopathy, diabetic nephropathy, renal tubulointerstitial fibrosis, and focal segmental glomerulosclerosis.

In some embodiments, the fibrosis is fibrosis of the lung resulting from conditions including but not limited to pulmonary interstitial fibrosis, sarcoidosis, pulmonary fibrosis, idiopathic pulmonary fibrosis, asthma, chronic obstructive pulmonary disease, diffuse alveolar damage disease, pulmonary hypertension, neonatal bronchopulmonary dysplasia, chronic asthma, and emphysema. There are several subnames or synonyms for pulmonary fibrosis including, but not limited to, cryptogenic fibrosing alveolitis, diffuse interstitial fibrosis, idiopathic interstitial pneumonitis, Hamman Rich syndrome, silicosis, asbestosis, berylliosis, coal worker's pneumoconiosis, coal miner's disease, miner's asthma, anthracosis, and anthracosilicosis.

In some embodiments, the fibrosis is fibrosis of the heart or pericardium resulting from conditions including but not limited to myocardial fibrosis, atherosclerosis, coronary artery restenosis, congestive cardiomyopathy, heart failure, and other post-ischemic conditions.

In some embodiments, the fibrosis is fibrosis of the eye resulting from conditions including but not limited to macular degeneration, exophthalmos of Grave's disease, proliferative vitreoretinopathy, anterior capsule cataract, corneal fibrosis, corneal scarring due to surgery, trabeculectomy-induced fibrosis, progressive sub-retinal fibrosis, multifocal granulomatous chorioretinitis, fibrosis due to wide angle glaucoma trabeculotomy, and other eye fibrosis.

In some embodiments, the fibrosis is fibrosis of the brain resulting from conditions including but not limited to glial scar tissue.

In some embodiments, the fibrosis is fibrosis of the skin resulting from conditions including but not limited to Depuytren's contracture, Scleroderma, keloid scarring, psoriasis, hyper-trophic scarring due to burns, atherosclerosis, restenosis, and psuedoscleroderma caused by spinal cord injury.

In some embodiments, the fibrosis is fibrosis of tissue including but not limited to the mouth or esophagus, pancreas, gastrointestinal tract, breast, bone, bone marrow, genitourinary system.

In some embodiments, a method for treatment and prevention of sinusoidal obstruction syndrome (SOS) or complications associated therewith is disclosed, the method comprising administering to a subject in need thereof an effective amount of at least one compound of Formula (I) and/or a pharmaceutical composition comprising at least one compound of Formula (I).

In some embodiments, a compound of Formula (I) and/or a pharmaceutical composition comprising at least one compound of Formula (I) may be used for the preparation and/or manufacture of a medicament for use in treating and/or preventing at least one of the diseases, disorders, and/or conditions described herein.

Whenever a term in the specification is identified as a range (e.g., C₁₋₄ alkyl) or “ranging from”, the range independently discloses and includes each element of the range. As a non-limiting example, C₁₋₄ alkyl groups includes, independently, C₁ alkyl groups, C₂ alkyl groups, C₃ alkyl groups, and C₄ alkyl groups. As another non-limiting example, “n is an integer ranging from 0 to 2” includes, independently, 0, 1, and 2.

The term “at least one” refers to one or more, such as one, two, etc. For example, the term “at least one C₁₋₄ alkyl group” refers to one or more C₁₋₄ alkyl groups, such as one C₁₋₄ alkyl group, two C₁₋₄ alkyl groups, etc.

The term “alkyl” includes saturated straight, branched, and cyclic (also identified as cycloalkyl), primary, secondary, and tertiary hydrocarbon groups. Non-limiting examples of alkyl groups include methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless stated otherwise specifically in the specification, an alkyl group may be optionally substituted.

The term “alkenyl” includes straight, branched, and cyclic hydrocarbon groups comprising at least one double bond. The double bond of an alkenyl group can be unconjugated or conjugated with another unsaturated group. Non-limiting examples of alkenyl groups include vinyl, allyl, butenyl, pentenyl, hexenyl, butadienyl, pentadienyl, hexadienyl, 2-ethyl hexenyl, and cyclopent-1-en-1-yl. Unless stated otherwise specifically in the specification, an alkenyl group may be optionally substituted.

The term “alkynyl” includes straight and branched hydrocarbon groups comprising at least one triple bond. The triple bond of an alkynyl group can be unconjugated or conjugated with another unsaturated group. Non-limiting examples of alkynyl groups include ethynyl, propynyl, butynyl, pentynyl, and hexynyl, Unless stated otherwise specifically in the specification, an alkynyl group may be optionally substituted.

The term “aryl” includes hydrocarbon ring system groups comprising at least 6 carbon atoms and at least one aromatic ring. The aryl group may be a monocyclic, bicyclic, tricyclic or tetracyclic ring system, which may include fused or bridged ring systems. Non-limiting examples of aryl groups include aryl groups derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted.

The terms “E-selectin antagonist” and “E-selectin inhibitor” are used interchangeably herein, and include inhibitors of E-selectin only, as well as inhibitors of E-selectin and either P-selectin or L-selectin, and inhibitors of E-selectin, P-selectin, and L-selectin.

The terms “galectin-3 antagonist” and “glectin-3 inhibitor” are used interchangeably herein, and include inhibitors of galectin-3 only, as well as inhibitors of galectin-3 and one or more other galectin, such as galectin-1, galectin-2, galectin-4, galectin-5, galectin-6, galectin-7, galectin-8, galectin-9, galectin-10, galectin-11, and galectin-12.

The term “glycomimetic” includes any naturally occurring or non-naturally occurring carbohydrate compound in which at least one substituent has been replaced, or at least one ring has been modified (e.g., substitution of carbon for a ring oxygen), to yield a compound that is not fully carbohydrate.

The term “halo” or “halogen” includes fluoro, chloro, bromo, and iodo.

The term “haloalkyl” includes alkyl groups, as defined herein, substituted by at least one halogen, as defined herein. Non-limiting examples of haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl. A “fluoroalkyl” is a haloalkyl wherein at least one halogen is fluoro. Unless stated otherwise specifically in the specification, a haloalkyl group may be optionally substituted.

The term “haloalkenyl” includes alkenyl groups, as defined herein, substituted by at least one halogen, as defined herein. Non-limiting examples of haloalkenyl groups include fluoroethenyl, 1,2-difluoroethenyl, 3-bromo-2-fluoropropenyl, and 1,2-dibromoethenyl. A “fluoroalkenyl” is a haloalkenyl substituted with at least one fluoro group. Unless stated otherwise specifically in the specification, a haloalkenyl group may be optionally substituted.

The term “haloalkynyl” includes alkynyl groups, as defined herein, substituted by at least one halogen, as defined herein. Non-limiting examples include fluoroethynyl, 1,2-difluoroethynyl, 3-bromo-2-fluoropropynyl, and 1,2-dibromoethynyl. A “fluoroalkynyl” is a haloalkynyl wherein at least one halogen is fluoro. Unless stated otherwise specifically in the specification, a haloalkynyl group may be optionally substituted.

The term “heterocyclyl” or “heterocyclic ring” includes 3- to 24-membered saturated or partially unsaturated non-aromatic ring groups comprising 2 to 23 ring carbon atoms and 1 to 8 ring heteroatom(s) each independently chosen from N, O, and S. Unless stated otherwise specifically in the specification, the heterocyclyl groups may be monocyclic, bicyclic, tricyclic or tetracyclic ring systems, which may include fused or bridged ring systems, and may be partially or fully saturated; any nitrogen, carbon or sulfur atom(s) in the heterocyclyl group may be optionally oxidized; any nitrogen atom in the heterocyclyl group may be optionally quaternized; and the heterocyclyl group. Non-limiting examples of heterocyclic ring include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

The term “heteroaryl” includes 5- to 14-membered ring groups comprising 1 to 13 ring carbon atoms and 1 to 6 ring heteroatom(s) each independently chosen from N, O, and S, and at least one aromatic ring. Unless stated otherwise specifically in the specification, the heteroaryl group may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Non-limiting examples include azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group may be optionally substituted.

The term “pharmaceutically acceptable salts” includes both acid and base addition salts. Non-limiting examples of pharmaceutically acceptable acid addition salts include chlorides, bromides, sulfates, nitrates, phosphates, sulfonates, methane sulfonates, formates, tartrates, maleates, citrates, benzoates, salicylates, and ascorbates. Non-limiting examples of pharmaceutically acceptable base addition salts include sodium, potassium, lithium, ammonium (substituted and unsubstituted), calcium, magnesium, iron, zinc, copper, manganese, and aluminum salts. Pharmaceutically acceptable salts may, for example, be obtained using standard procedures well known in the field of pharmaceuticals.

The term “prodrug” includes compounds that may be converted, for example, under physiological conditions or by solvolysis, to a biologically active compound described herein. Thus, the term “prodrug” includes metabolic precursors of compounds described herein that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release the active compound(s) as described herein in vivo when such prodrug is administered to a subject. Non-limiting examples of prodrugs include ester and amide derivatives of hydroxy, carboxy, mercapto and amino functional groups in the compounds described herein.

The term “substituted” includes the situation where, in any of the above groups, at least one hydrogen atom is replaced by a non-hydrogen atom such as, for example, a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also includes the situation where, in any of the above groups, at least one hydrogen atom is replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles.

The present disclosure includes within its scope all the possible geometric isomers, e.g., Z and E isomers (cis and trans isomers), of the compounds as well as all the possible optical isomers, e.g., diastereomers and enantiomers, of the compounds. Furthermore, the present disclosure includes in its scope both the individual isomers and any mixtures thereof, e.g., racemic mixtures. The individual isomers may be obtained using the corresponding isomeric forms of the starting material or they may be separated after the preparation of the end compound according to conventional separation methods. For the separation of optical isomers, e.g., enantiomers, from the mixture thereof conventional resolution methods, e.g., fractional crystallization, may be used.

The present disclosure includes within its scope all possible tautomers. Furthermore, the present disclosure includes in its scope both the individual tautomers and any mixtures thereof.

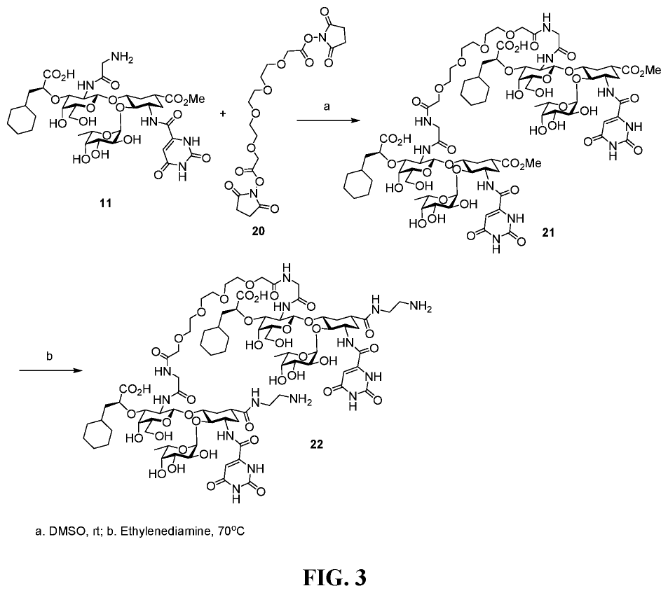

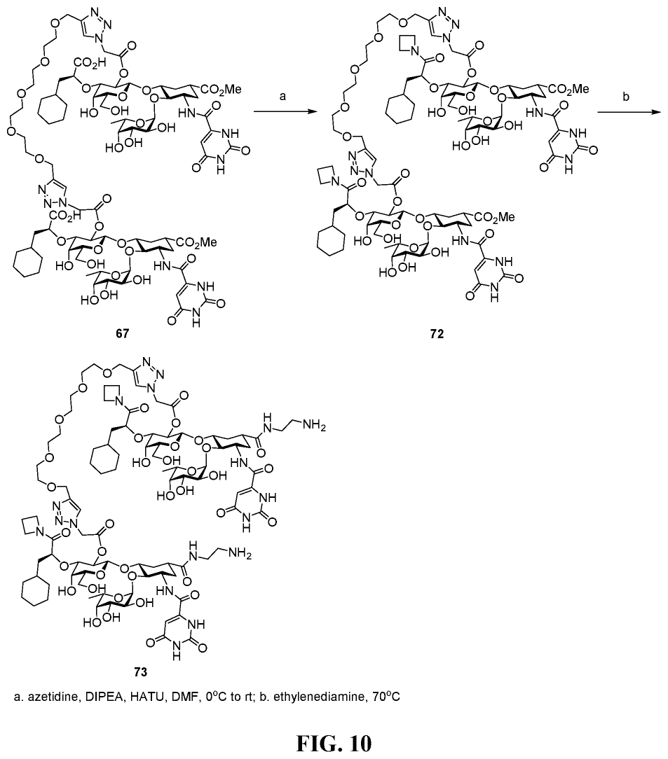

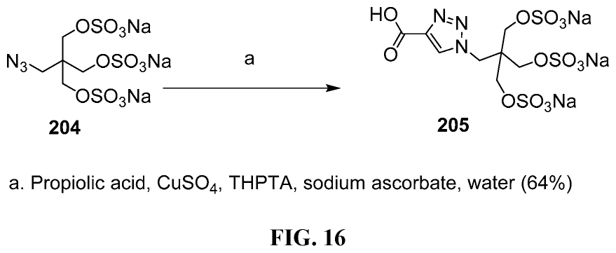

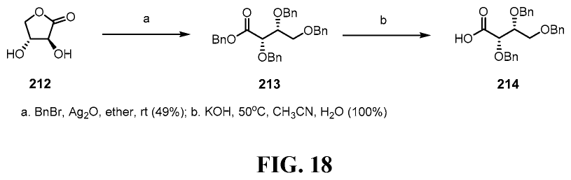

Compounds of Formula (I) may be prepared as shown in, for example, FIGS. 3-6, 9-15, 17-20, 23-26, and 28-29. It is understood that one of ordinary skill in the art may be able to make these compounds by similar methods or by combining other methods known to one of ordinary skill in the art. It is also understood that one of ordinary skill in the art would be able to make other compounds of Formula (I) not specifically illustrated herein by using appropriate starting components and modifying the parameters of the synthesis as needed (e.g., see FIG. 16). In general, starting components may be obtained from sources such as Sigma Aldrich, Alfa Aesar, Maybridge, Matrix Scientific, TCI, and Fluorochem USA, etc. and/or synthesized according to sources known to those of ordinary skill in the art (see, for example, Advanced Organic Chemistry: Reactions, Mechanisms, and Structure, 5th edition (Wiley, December 2000)) and/or prepared as described herein.

It will also be appreciated by those skilled in the art that in the processes described herein the functional groups of intermediate compounds may need to be protected by suitable protecting groups, even if not specifically described. Such functional groups include hydroxy, amino, mercapto, and carboxylic acid. Suitable protecting groups for hydroxy include but are not limited to trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include but are not limited to t-butoxycarbonyl, benzyloxycarbonyl, and the like. Suitable protecting groups for mercapto include but are not limited to —C(O)R″ (where R″ is alkyl, aryl or arylalkyl), p-methoxybenzyl, trityl and the like. Suitable protecting groups for carboxylic acid include but are not limited to alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

Analogous reactants to those described herein may be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002.

Methods known to one of ordinary skill in the art may be identified through various reference books, articles, and databases. Suitable reference books and treatise that detail the synthesis of reactants useful in the preparation of compounds of the present disclosure, or provide references to articles that describe the preparation, include for example, “Synthetic Organic Chemistry,” John Wiley & Sons, Inc., New York; S. R. Sandier et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure,” 4th Ed., Wiley-Interscience, New York, 1992. Additional suitable reference books and treatise that detail the synthesis of reactants useful in the preparation of compounds of the present disclosure, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Biological activity of a compound described herein may be determined, for example, by performing at least one in vitro and/or in vivo study routinely practiced in the art and described herein or in the art. In vitro assays include without limitation binding assays, immunoassays, competitive binding assays, and cell based activity assays.

An inhibition assay may be used to screen for antagonists of E-selectin. For example, an assay may be performed to characterize the capability of a compound described herein to inhibit (i.e., reduce, block, decrease, or prevent in a statistically or biologically significant manner) interaction of E-selectin with sLe^{a} or sLe^{x}. The inhibition assay may be a competitive binding assay, which allows the determination of IC₅₀ values. By way of example, E-selectin/Ig chimera may be immobilized onto a matrix (e.g., a multi-well plate, which may be made from a polymer, such as polystyrene; a test tube, and the like); a composition may be added to reduce nonspecific binding (e.g., a composition comprising non-fat dried milk or bovine serum albumin or other blocking buffer routinely used by a person skilled in the art); the immobilized E-selectin may be contacted with the candidate compound in the presence of sLe^{a} comprising a reporter group under conditions and for a time sufficient to permit sLe^{a} to bind to the immobilized E-selectin; the immobilized E-selectin may be washed; and the amount of sLe^{a} bound to immobilized E-selectin may be detected. Variations of such steps can be readily and routinely accomplished by a person of ordinary skill in the art.

An inhibition assay may be used to screen for antagonists of galectin-3. For example, an assay may be performed to characterize the capability of a compound described herein to inhibit interaction of galectin-3 with a Galβ1-3GlcNAc carbohydrate structure. The inhibition assay may be a competitive binding assay, which allows the determination of IC₅₀ values. By way of example, a Galβ1-3GlcNAc polymer may be immobilized onto a matrix; a composition may be added to reduce nonspecific binding; the immobilized Galβ1-3GlcNAc polymer may be contacted with the candidate compound in the presence of galectin-3 group under conditions and for a time sufficient to permit galectin-3 to bind to the immobilized Galβ1-3GlcNAc polymer; the immobilized Galβ1-3GlcNAc polymer may be washed; and the amount of galectin-3 bound to the immobilized Galβ1-3GlcNAc polymer may be detected. Variations of such steps can be readily and routinely accomplished by a person of ordinary skill in the art.

An inhibition assay may be used to screen for antagonism of CXCR4 mediated chemotaxis. For example, an assay may be performed to measure the ability of a glycomimetic CXCR4 antagonist to inhibit migration of CCRF-CEM cells, which express CXCR4 on their cell surfaces, across a membrane toward the CXCR4 ligand CXCL12 (SDF-1α). By way of example, CCRF-CEM cells are human T lymphoblasts that express CXCR4 on the cell surface. The cells may be labeled with 3 uM Calcein AM to enable detection by fluorescence. The cells may be treated with a CXCR4 antagonist and placed into the upper chamber of a transwell insert. The transwells may be placed into the wells of a 24-well plate with each well containing 600 ul of RPMI 1640 plus 2% FBS and 50 ng/mL CXCL12 (SDF1α). The cells may be allowed to migrate across the membrane from the upper chamber into the lower chamber for 3 hours at 37° C. in 5% CO2. The transwell inserts may be removed from the 24-well plate and the fluorescence in the lower chambers measured using a Molecular Devices FlexStation 3 with an excitation wavelength of 485 nm and an emission wavelength of 538 nm.

Alternatively, an assay may be used to measure the ability of a glycomimetic CXCR4 antagonist to inhibit the binding of CXCL12 (SDF-1α) to CHO cells that have been genetically engineered to express CXCR4 on the cell surface. One skilled in the art may activate CXCR4 by ligand binding (CXCL12), causing Gi to dissociate from the CXCR4 complex. The activated CXCR4 may bind to adenylyl cyclase, thus inactivating it, resulting in decreased levels of intracellular cAMP. Intracellular cAMP is usually low, so the decrease of the low level of cAMP by a Gi-coupled receptor will be hard to detect. Forskolin is added to the CHO cells to directly activate adenylyl cyclase (bypassing all GPCRs), thus raising the level of cAMP in the cell, so that a Gi response can be easily observed. CXCL12 interaction with CXCR4 decreases the intracellular level of cAMP and inhibition of CXCL12 interaction with CXCR4 by a CXCR4 antagonist increases the intracellular cAMP level, which is measured by luminescence.

Alternatively, one skilled in the art may use an assay to measure the ability of a glycomimetic CXCR4 antagonist to block the binding of an anti-CXCR4 antibody to Jurkat cells, which express CXCR4 on the cell surface. Jurkat cells may be treated with a CXCR4 antagonist followed by a phycoerythrin-conjugated anti-CXCR4 antibody. The antibody may be allowed to bind to the cells for 1 hour at 4° C. The cells may be washed and the binding of the anti-CXCR4-PE antibody to the cells may be assessed by flow cytometry.

Conditions for a particular assay include temperature, buffers (including salts, cations, media), and other components that maintain the integrity of any cell used in the assay and the compound, which a person of ordinary skill in the art will be familiar and/or which can be readily determined. A person of ordinary skill in the art also readily appreciates that appropriate controls can be designed and included when performing the in vitro methods and in vivo methods described herein.

The source of a compound that is characterized by at least one assay and techniques described herein and in the art may be a biological sample that is obtained from a subject who has been treated with the compound. The cells that may be used in the assay may also be provided in a biological sample. A “biological sample” may include a sample from a subject, and may be a blood sample (from which serum or plasma may be prepared), a biopsy specimen, one or more body fluids (e.g., lung lavage, ascites, mucosal washings, synovial fluid, urine), bone marrow, lymph nodes, tissue explant, organ culture, or any other tissue or cell preparation from the subject or a biological source. A biological sample may further include a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. In some embodiments, the subject or biological source may be a human or non-human animal, a primary cell culture (e.g., immune cells), or culture adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, and the like.

As described herein, methods for characterizing E-selectin, galectin-3, and/or CXCR4 chemokine receptor antagonists include animal model studies. Non-limiting examples of animal models for liquid cancers used in the art include multiple myeloma (see, e.g., DeWeerdt, Nature 480:S38-S39 (15 Dec. 2011) doi:10.1038/480S38a; Published online 14 Dec. 2011; Mitsiades et al., Clin. Cancer Res. 2009 15:1210021 (2009)); acute myeloid leukemia (AML) (Zuber et al., Genes Dev. 2009 April 1; 23(7): 877-889). Animal models for acute lymphoblastic leukemia (ALL) have been used by persons of ordinary skill in the art for more than two decades. Numerous exemplary animal models for solid tumor cancers are routinely used and are well known to persons of ordinary skill in the art.

The compounds of the present disclosure and the pharmaceutical compositions comprising at least one of such compounds may be useful in methods for treating and/or preventing a disease or disorder that is treatable by inhibiting at least one activity of E-selectin, galectin-3, and CXCR4 chemokine receptors, or any combination thereof (and/or inhibiting binding of E-selectin, galectin-3, and CXCR4 chemokine receptors to ligand(s), which in turn inhibits a biological activity).

The compounds of the present disclosure and pharmaceutical compositions comprising at least one such compound may be useful in methods for treating and/or preventing at least one inflammatory disease. Inflammation comprises reaction of vascularized living tissue to injury. By way of example, although E-selectin, galectin-3, and CXCR4 chemokine receptors mediated cell adhesion may be important to the body's anti-infective immune response, in other circumstances, E-selectin, galectin-3, and CXCR4 chemokine receptors mediated cell adhesion may be undesirable or excessive, resulting in tissue damage and/or scarring instead of repair. For example, many pathologies (such as autoimmune and inflammatory diseases, shock and reperfusion injuries) involve abnormal adhesion of white blood cells. Therefore, inflammation affects blood vessels and adjacent tissues in response to an injury or abnormal stimulation by a physical, chemical, or biological agent. Examples of inflammatory diseases, disorders, or conditions include, without limitation, dermatitis, chronic eczema, psoriasis, multiple sclerosis, rheumatoid arthritis, systemic lupus erythematosus, graft versus host disease, sepsis, diabetes, atherosclerosis, Sjogren's syndrome, progressive systemic sclerosis, scleroderma, acute coronary syndrome, ischemic reperfusion, Crohn's disease, inflammatory bowel disease, endometriosis, glomerulonephritis, myasthenia gravis, idiopathic pulmonary fibrosis, asthma, allergic reaction, acute respiratory distress syndrome (ARDS) or other acute leukocyte-mediated lung injury, vasculitis, or inflammatory autoimmune myositis. Other diseases and disorders for which the compounds described herein may be useful for treating and/or preventing include hyperactive coronary circulation, microbial infection, cancer metastasis, thrombosis, wounds, burns, spinal cord damage, digestive tract mucous membrane disorders (e.g., gastritis, ulcers), osteoporosis, osteoarthritis, septic shock, traumatic shock, stroke, nephritis, atopic dermatitis, frostbite injury, adult dyspnoea syndrome, ulcerative colitis, diabetes and reperfusion injury following ischemic episodes, prevention of restenosis associated with vascular stenting, and for undesirable angiogenesis, for example, angiogenesis associated with tumor growth.

As discussed in detail herein, a disease or disorder to be treated or prevented is a cancer and related metastasis and includes cancers that comprise solid tumor(s) and cancers that comprise liquid tumor(s). The compounds of the present disclosure and pharmaceutical compositions comprising at least one such compound may be useful in methods for preventing and/or treating cancer. In some embodiments, the at least one compound may be used for treating and/or preventing metastasis and/or for inhibiting (slowing, retarding, or preventing) metastasis of cancer cells.

In some embodiments, at least one compound of Formula (I) and/or a pharmaceutical composition comprising at least one compound of Formula (I) is administered to a cancer patient in remission. In some embodiments, the at least one compound of Formula (I) and/or a pharmaceutical composition comprising at least one compound of Formula (I) is administered as a cancer vaccine to stimulate marrow infiltrating lymphocytes (“MILs”) in a cancer patient or cancer survivor to prevent relapse.

In some embodiments, a method of treating cancer and/or preventing a cancer relapse is disclosed, wherein the method comprises administering to a patient in need thereof an effective amount of at least one compound of Formula (I) and/or a pharmaceutical composition comprising at least one compound of Formula (I), wherein the amount of compound of Formula (I) administered is sufficient to mobilize MILs of the patient into the peripheral blood.

In some embodiments, a method of treating cancer and/or preventing a cancer relapse is provided comprising administering to a donor patient at least one compound of Formula (I) and/or a pharmaceutical composition comprising at least one compound of Formula (I) in an amount of sufficient to mobilize MILs of the patient out of the marrow (e.g., into the peripheral blood), recovering MILS (e.g., recovering them from the peripheral blood), and transplanting at least a portion of the MIL cell population to the donor patient or another patient. In some embodiments, the MIL cell population is expanded ex vivo before transplantation.

In some embodiments, a method of preventing cancer is provided comprising administering to a donor patient at least one compound of Formula (I) and/or a pharmaceutical composition comprising at least one compound of Formula (I) in an amount sufficient to mobilize MILs of the patient out of the bone marrow (e.g., into the peripheral blood), recovering MILs (e.g., recovering them from the peripheral blood), and transplanting at least a portion of MIL cell population to a subject (e.g., a non-cancer patient, a patient suffering from a different form or type of cancer than the donor patient, etc.). In some embodiments, the MIL cell population is expanded ex vivo before transplantation.

In some embodiments, the compounds of present disclosure and pharmaceutical compositions comprising at least one such compound may be used for decreasing (i.e., reducing) the likelihood of occurrence of metastasis of cancer cells in an individual (i.e., subject, patient) who is in need thereof. The compounds of the present disclosure and compositions comprising at least one such compound may be used for decreasing (i.e., reducing) the likelihood of occurrence of infiltration of cancer cells into bone marrow in an individual who is in need thereof. The individuals (or subjects) in need of such treatments include subjects who have been diagnosed with a cancer, which includes cancers that comprise solid tumor(s) and cancers that comprise liquid tumor(s).

The compounds of the present disclosure and pharmaceutical compositions comprising at least one such compound may be administered as an adjunct therapy to chemotherapy and/or radiotherapy, which is/are being delivered to the subject as primary therapy for treating the cancer. The chemotherapy and/or radiotherapy that may be administered depend upon several factors including the type of cancer, location of the tumor(s), stage of the cancer, age and gender and general health status of the subject. A person of ordinary skill in the medical art can readily determine the appropriate chemotherapy regimen and/or radiotherapy regimen for the subject in need. The person of ordinary skill in the medical art can also determine, with the aid of preclinical and clinical studies, when the compound of the present disclosure or pharmaceutical composition comprising at least one such compound should be administered to the subject, that is whether the compound or composition is administered prior to, concurrent with, or subsequent to a cycle of the primary chemotherapy or radiation treatment.

Also provided herein is a method for inhibiting adhesion of a tumor cell that expresses a ligand of E-selectin to an endothelial cell expressing E-selectin on its cell surface, which method comprises contacting the endothelial cell with at least one compound of the present disclosure or pharmaceutical compositions comprising at least one such compound, thereby permitting the compound to interact with E-selectin on the endothelial cell surface and inhibiting binding of the tumor cell to the endothelial cell. Without wishing to be bound by theory, inhibiting adhesion of tumor cells to endothelial cells may reduce in a significant manner, the capability of the tumor cells to extravasate into other organs, blood vessels, lymph, or bone marrow and thereby reduce, decrease, or inhibit, or slow the progression of the cancer, including reducing, decreasing, inhibiting, or slowing metastasis.

In some embodiments, a method for inhibiting activation of hepatic and/or pancreatic stellate cells is disclosed, the method comprising administering at least one compound of Formula (I) and/or a pharmaceutical composition comprising at least one compound of Formula (I).

In some embodiments, a method for inhibiting adhesion of metastasized tumor cells is disclosed, the method comprising administering at least one compound of Formula (I) and/or a pharmaceutical composition comprising at least one compound of Formula (I).

In some embodiments, a method for inhibiting cell-cell interactions and/or interactions between cells and the extracellular matrix where the cell-cell interactions and cell-matrix are induced by galectin-3 molecules bound carbohydrates found on the surface of cells is disclosed, the method comprising administering at least one compound of Formula (I) and/or a pharmaceutical composition comprising at least one compound of Formula (I). In some embodiments, the cells are tumor cells and cell-cell interactions and/or cell-matrix are responsible for the development of at least one tumor disease.

In some embodiments, a method for reducing the rate of growth of tumor cells which express galectin-3 is disclosed, the method comprising administering at least one compound of Formula (I) and/or a pharmaceutical composition comprising at least one compound of Formula (I). In some embodiments, the level of at least one G1/S cyclin in the tumor cell is reduced.

As described herein, at least one of the compounds of the present disclosure or pharmaceutical compositions comprising at least one such compound may be administered in combination with at least one additional anti-cancer agent. Chemotherapy may comprise one or more chemotherapeutic agents. For example, chemotherapy agents, radiotherapeutic agents, inhibitors of phosphoinositide-3 kinase (PI3K), and inhibitors of VEGF may be used in combination with a compound of Formula (I) described herein. Non-limiting examples of inhibitors of PI3K include the compound named by Exelixis as “XL499.” Non-limiting examples of VEGF inhibitors include the compound called “cabo” (previously known as XL184). Many other chemotherapeutics are small organic molecules. As understood by a person of ordinary skill in the art, chemotherapy may also refer to a combination of two or more chemotherapeutic molecules that are administered coordinately and which may be referred to as combination chemotherapy. Numerous chemotherapeutic drugs are used in the oncology art and include, for example, alkylating agents; antimetabolites; anthracyclines, plant alkaloids; and topoisomerase inhibitors.

The compounds of the present disclosure or pharmaceutical compositions comprising at least one such compound may function independently from the anti-cancer agent or may function in coordination with the anti-cancer agent, e.g., by enhancing effectiveness of the anti-cancer agent or vice versa. Accordingly, provided herein are methods for enhancing (i.e., enhancing, promoting, improving the likelihood of, enhancing in a statistically or biologically significant manner) and/or maintaining survival of hematopoietic stem cells (HSC) in a subject who is treated with and/or will be treated with a chemotherapeutic drug(s) and/or radioactive therapy, respectively, comprising administering at least one compound of Formula (I) as described herein. In some embodiments, the subject receives and/or will receive both chemotherapy and radiation therapy. Also, provided herein is a method for reducing (i.e., reducing, inhibiting, diminishing in a statistically or biologically significant manner) chemosensitivity and/or radiosensitivity of hematopoietic stem cells (HSC) to the chemotherapeutic drug(s) and/or radioactive therapy, respectively, in a subject. Because repeated cycles of chemotherapy and radiotherapy often diminish the ability of HSCs to recover and replenish bone marrow, the glycomimetic compounds described herein may be useful for subjects who will receive more than one cycle, such as at least two, three, four or more cycles, of chemotherapy and/or radiotherapy. HSCs reside in the bone marrow and generate the cells that are needed to replenish the immune system and the blood. Anatomically, bone marrow comprises a vascular niche that is adjacent to bone endothelial sinuses (see, e.g., Kiel et al., Cell 121:1109-21 (2005); Sugiyama et al., Immunity 25:977-88 (2006); Mendez-Ferrer et al., Nature 466:829-34 (2010); Butler et al., Cell Stem Cell 6:251-64 (2010)). A recent study describes that E-selectin promotes HSC proliferation and is an important component of the vascular niche (see, e.g., Winkler et al., Nature Medicine published online 21 Oct. 2012; doi:10.1038/nm.2969). Deletion or inhibition of E-selectin enhanced HSC survival in mice that were treated with chemotherapeutic agents or radiotherapy and accelerated blood neutrophil recovery (see, e.g., Winkler et al., supra). Additionally, galectin-3 has recently been reported to interfere with hematopoiesis and promote terminal differentiation of myeloid progenitors (see, e.g., Brand et al., Cell Tissue Res 346:427-37 (2011)).

In addition, the administration of at least one compound of the present disclosure or pharmaceutical composition comprising at least one such compounds may be in conjunction with one or more other therapies, e.g., for reducing toxicities of therapy. For example, at least one palliative agent to counteract (at least in part) a side effect of a therapy (e.g., anti-cancer therapy) may be administered. Agents (chemical or biological) that promote recovery, or counteract side effects of administration of antibiotics or corticosteroids, are examples of such palliative agents. At least one compound described herein may be administered before, after, or concurrently with administration of at least one additional anti-cancer agent or at least one palliative agent to reduce a side effect of therapy. When administration is concurrent, the combination may be administered from a single container or two (or more) separate containers.

Cancer cells (also called herein tumor cells) that may be prevented (i.e., inhibited, slowed) from metastasizing, from adhering to an endothelial cell, or from infiltrating bone marrow include cells of solid tumors and liquid tumors (including hematological malignancies). Examples of solid tumors are described herein and include colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, melanoma, breast cancer, and pancreatic cancer. Liquid tumors occur in the blood, bone marrow, and lymph nodes and include leukemia (e.g., AML, ALL, CLL, and CML), lymphoma (e.g., Hodgkin lymphoma and non-Hodgkin lymphoma), and myeloma (e.g., multiple myeloma). As used herein, the term cancer cells include mature, progenitor, and cancer stem cells.

Bones are a common location for cancer to infiltrate once leaving the primary tumor location. Once cancer resides in bone, it is frequently a cause of pain to the individual. In addition, if the particular bone affected is a source for production of blood cells in the bone marrow, the individual may develop a variety of blood cell related disorders. Breast and prostate cancer are examples of solid tumors that migrate to bones. Acute myelogenous leukemia (AML) and multiple myeloma (MM) are examples of liquid tumors that migrate to bones. Cancer cells that migrate to bone will typically migrate to the endosteal region of the bone marrow. Once cancer cells have infiltrated into the marrow, the cells become quiescent and are protected from chemotherapy. The compounds of the present disclosure may block infiltration of disseminated cancer cells into bone marrow. A variety of subjects may benefit from treatment with the compounds. Examples of such subjects include individuals with a cancer type having a propensity to migrate to bone where the tumor is still localized or the tumor is disseminated but not yet infiltrated bone, or where individuals with such a cancer type are in remission.

The cancer patient population most likely to respond to treatment using antagonists of E-selectin, galectin-3, and CXCR4 chemokine receptors (e.g., compounds of Formula (I)) described herein can be identified based on the mechanisms of action of E-selectin. For example, patients may be selected that express a highly active E-selectin as determined by the genetic polymorphism for E-selectin of S128R (Alessandro et al., Int. J. Cancer 121:528-535, 2007). In addition, patients for treatment by the compounds described herein may also selected based on elevated expression of the E-selectin binding ligands (sialyl Le^{a} and sialyl Le^{x}) as determined by antibodies directed against cancer-associated antigens CA-19-9 (Zheng et al., World J. Gastroenterol. 7:431-434, 2001) and CD65. In addition, antibodies HECA-452 and FH-6 which recognize similar carbohydrate ligands of E-selectin may also be used in a diagnostic assay to select the cancer patient population most likely to respond to this treatment. Likewise, pateints may be identified for treatment based on levels of galectin-3 detected in serum or plasma by a diagnostic assay such as the Abbott Laboratories ARCHITECT Galectin-3 assay, which can be used for determining galectin-3 in serum or plasma to stratify heart failure patients for proper treatment.

The compounds of the present disclosure and pharmaceutical compositions comprising at least one such compound may be useful in methods for mobilizing cells from the bone marrow to the peripheral vasculature and tissues. As discussed herein, in some embodiments, the compounds and compositions are useful for mobilizing hematopoietic cells, including hematopoietic stem cells and hematopoietic progenitor cells. In some embodiments, the compounds act as mobilizing agents of normal blood cell types. In some embodiments, the agents are used in methods for mobilizing mature white blood cells (which may also be called leukocytes herein), such as granulocytes (e.g., neutrophils, eosinophils, basophils), lymphocytes, and monocytes from the bone marrow or other immune cell compartments such as the spleen and liver. Methods are also provided for using the compounds of the present disclosure and pharmaceutical compositions comprising at least one such compound in methods for mobilizing tumor cells from the bone marrow. The tumor cells may be malignant cells (e.g., tumor cells that are metastatic cancer cells, or highly invasive tumor cells) in cancers. These tumor cells may be of hematopoietic origin or may be malignant cells of another origin residing in the bone.

In some embodiments, the methods using the compounds described herein are useful for mobilizing hematopoietic cells, such as hematopoietic stem cells and progenitor cells and leukocytes (including granulocytes such as neutrophils), which are collected (i.e., harvested, obtained) from the subject receiving a compound of Formula (I) and at a later time are administered back into the same subject (autologous donor) or administered to a different subject (allogeneic donor). Hematopoietic stem cell replacement and hematopoietic stem cell transplantation have been successfully used for treating a number of diseases (including cancers) as described herein and in the art. By way of example, stem cell replacement therapy or transplantation follows myeloablation of a subject, such as occurs with administration of high dose chemotherapy and/or radiotherapy. Desirably, an allogeneic donor shares sufficient HLA antigens with the recipient/subject to minimize the risk of host versus graft disease in the recipient (i.e., the subject receiving the hematopoietic stem cell transplant). Obtaining the hematopoietic cells from the donor subject (autologous or allogeneic) is performed by apheresis or leukapheresis. HLA typing of a potential donor and the recipient and apheresis or leukapheresis are methods routinely practiced in the clinical art.

By way of non-limiting example, autologous or allogenic hematopoietic stem cells and progenitors cells may be used for treating a recipient subject who has certain cancers, such as Hodgkin lymphoma, non-Hodgkin lymphoma, or multiple myeloma. Allogeneic hematopoietic stem cells and progenitors cells may be used, for example, for treating a recipient subject who has acute leukemia (e.g., AML, ALL); chronic lymphocytic leukemia (CLL); amegakaryocytosis/congenital thrombocytopenia; aplastic anemia/refractory anemia; familial erythrophagocytic lymphohistiocytosis; myelodysplastic syndrome/other myelodysplastic disorders: osteopetrosis; paroxysmal nocturnal hemoglobinuria; and Wiskott-Aldrich syndrome, for example. Exemplary uses for autologous hematopoietic stem cells and progenitors cells include treating a recipient subject who has amyloidosis; germ cell tumors (e.g., testicular cancer); or a solid tumor. Allogeneic hematopoietic stem cell transplants have also been investigated for use in treating solid tumors (see, e.g., Ueno et al., Blood 102:3829-36 (2003)).

In some embodiments of the methods described herein, the subject is not a donor of peripheral hematopoietic cells but has a disease, disorder, or condition for which mobilization of hematopoietic cells in the subject will provide clinical benefit. Stated another way, while this clinical situation is similar to autologous hematopoietic cell replacement, the mobilized hematopoietic cells are not removed and given back to the same subject at a later time as occurs, for example, with a subject who receives myeloablation therapy. Accordingly, methods are provided for mobilizing hematopoietic cells, such as hematopoietic stem cells and progenitor cells and leukocytes (including granulocytes, such as neutrophils), by administering at least once compound of Formula (I). Mobilizing hematopoietic stem cells and progenitor cells may be useful for treating an inflammatory condition or for tissue repair or wound healing. See, e.g., Mimeault et al., Clin. Pharmacol. Therapeutics 82:252-64 (2007).

In some embodiments, the methods described herein are useful for mobilizing hematopoietic leukocytes (white blood cells) in a subject, which methods may be used in treating diseases, disorders, and conditions for which an increase in white blood cells, such as neutrophils, eosinophils, lymphocytes, monocytes, basophils, will provide clinical benefit. For example, for cancer patients, the compounds of Formula (I) are beneficial for stimulating neutrophil production to compensate for hematopoietic deficits resulting from chemotherapy or radiation therapy. Other diseases, disorders, and conditions to be treated include infectious diseases and related conditions, such as sepsis. When the subject to whom at least one compound of Formula (I) is administered is a donor, neutrophils may be collected for administration to a recipient subject who has reduced hematopoietic function, reduced immune function, reduced neutrophil count, reduced neutrophil mobilization, severe chronic neutropenia, leucopenia, thrombocytopenia, anemia, and acquired immune deficiency syndrome. Mobilization of mature white blood cells may be useful in subjects to improve or to enhance tissue repair, and to minimize or prevent vascular injury and tissue damage, for example following liver transplantation, myocardial infarction or limb ischemia. See, e.g., Pelus, Curr. Opin. Hematol. 15:285-92 (2008); Lemoli et al., Haematologica 93:321-24 (2008).

The compounds of Formula (I) may be used in combination with one or more other agents that mobilize hematopoietic cells. Such agents include, for example, G-CSF; AMD3100 or other CXCR4 antagonists; GRO-β (CXCL2) and an N-terminal 4-amino truncated form (SB-251353); IL-8SDF-1α peptide analogs, CTCE-0021 and CTCE-0214; and the SDF1 analog, Met-SDF-1p (see, e.g., Pelus, supra and references cited therein). In some embodiments, a compound of Formula (I) may be administered with other mobilizing agents used in the art, which may permit administration of a lower dose of GCSF or AMD3100, for example, than required in the absence of a compound of Formula (I). The appropriate therapeutic regimen for administering a compound of Formula (I) in combination with another mobilizing agent or agents can be readily determined by a person skilled in the clinical art.

The compounds of the present disclosure and pharmaceutical compositions comprising at least one such compound may be useful in methods for preventing and/or treating mucositis. In some embodiments, at least one compound of Formula (I) and/or a pharmaceutical composition comprising at least one compound of Formula (I) may be used in methods described herein for decreasing the likelihood of occurrence of mucositis in a subject who is in need thereof by administering the compound or composition to the subject. In some embodiments, the mucositis is chosen from oral mucositis, esophageal mucositis, and gastrointestinal mucositis. In some embodiments, the mucositis is alimentary mucositis.

It is believed that approximately half of all cancer patients undergoing therapy suffer some degree of mucositis. Mucositis is believed to occur, for example, in virtually all patients treated with radiation therapy for head and neck tumors, all patients receiving radiation along the GI tract, and approximately 40% of those subjected to radiation therapy and/or chemotherapy for tumors in other locations (e.g., leukemias or lymphomas). It is also is believed to be highly prevalent in patients treated with high dose chemotherapy and/or irradiation for the purpose of myeloablation, such as in preparation for stem cell or bone marrow transplantation. The compounds of the present disclosure and pharmaceutical compositions comprising at least one such compound may be useful in methods for treating and/or preventing mucositis in a subject afflicted with cancer. In some embodiments, the subject is afflicted with a cancer chosen from head and neck cancer, breast cancer, lung cancer, ovarian cancer, prostate cancer, lymphatic cancer, leukemic cancer, and/or gastrointestinal cancer. In some embodiments, the mucositis is associated with radiation therapy and/or chemotherapy. In some embodiments, the chemotherapy comprises administering a therapeutically effective amount of at least one compound chosen from platinum, cisplatin, carboplatin, oxaliplatin, mechlorethamine, cyclophosphamide, chlorambucil, azathioprine, mercaptopurine, vincristine, vinblastine, vinorelbine, vindesine, etoposide, teniposide, paclitaxel, docetaxel, irinotecan, topotecan, amsacrine, etoposide, etoposide phosphate, teniposide, 5-fluorouracil (5-FU), leucovorin, methotrexate, gemcitabine, taxane, leucovorin, mitomycin C, tegafur-uracil, idarubicin, fludarabine, mitoxantrone, ifosfamide and doxorubicin.

In some embodiments, the method further comprises administering a therapeutically effective amount of at least one MMP inhibitor, inflammatory cytokine inhibitor, mast cell inhibitor, NSAID, NO inhibitor, or antimicrobial compound.

In some embodiments, the method further comprises administering a therapeutically effective amount of velafermin and/or palifermin.

In some embodiments, the method further comprises administering a therapeutically effective amount of Davanat®, mannose, and/or galactose.

The compounds of the present disclosure and pharmaceutical compositions comprising at least one such compound may be useful in methods for treating and/or preventing thrombosis. As described herein methods are provided for inhibiting formation of a thrombus or inhibiting the rate at which a thrombus is formed. These methods may therefore be used for preventing thrombosis (i.e., reducing or decreasing the likelihood of occurrence of a thrombus in a statistically or clinically significant manner).

Thrombus formation may occur in infants, children, teenagers and adults. An individual may have a hereditary predisposition to thrombosis. Thrombosis may be initiated, for example, due to a medical condition (such as cancer or pregnancy), a medical procedure (such as surgery) or an environmental condition (such as prolonged immobility). Other individuals at risk for thrombus formation include those who have previously presented with a thrombus.

The compounds of the present disclosure and pharmaceutical compositions comprising at least one such compound may be useful in methods for treating individuals undergoing thrombosis or who are at risk of a thrombotic event occurring. Such individuals may or may not have a risk of bleeding. In some embodiments, the individual has a risk of bleeding. In some embodiments, the thrombosis is a venous thromboembolism (VTE). VTE causes deep vein thrombosis and pulmonary embolism. Low molecular weight (LMW) heparin is the current mainstay therapy for the prevention and treatment of VTE. In many circumstances, however, the use of LMW heparin is contraindicated. LMW heparin is a known anti-coagulant and delays clotting over four times longer than control bleeding times. Patients undergoing surgery, patients with thrombocytopenia, patients with a history of stroke, and many cancer patients should avoid administration of heparin due to the risk of bleeding. By contrast, administration of the compounds of Formula (I) significantly reduces the time to clotting than occurs when LMW heparin is administered, and thus provide a significant improvement in reducing bleeding time compared with LMW heparin. Accordingly, the compounds and pharmaceutical compositions described herein may not only be useful for treating a patient for whom the risk of bleeding is not significant, but also may be useful in when the risk of bleeding is significant and the use of anti-thrombosis agents with anti-coagulant properties (such as LMW heparin) is contraindicated.

The compounds of the present disclosure and pharmaceutical compositions comprising at least one such compound may be administered in combination with at least one additional anti-thrombosis agent. The compounds of the present disclosure and pharmaceutical compositions comprising at least one such compound may function independently from the anti-thrombosis agent or may function in coordination with the at least one anti-thrombosis agent. In addition, the administration of one or more of the compounds or compositions may be in conjunction with one or more other therapies, e.g., for reducing toxicities of therapy. For example, at least one palliative agent to counteract (at least in part) a side effect of therapy may be administered. Agents (chemical or biological) that promote recovery and/or counteract side effects of administration of antibiotics or corticosteroids are examples of such palliative agents. The compounds of the present disclosure and pharmaceutical composition comprising at least one such compound may be administered before, after, or concurrently with administration of at least one additional anti-thrombosis agent or at least one palliative agent to reduce a side effect of therapy. Where administration is concurrent, the combination may be administered from a single container or two (or more) separate containers.

The compounds of the present disclosure and pharmaceutical compositions comprising at least one such compound may be useful for treating and/or preventing at least one cardiovascular disease, disorder and/or condition. Non-limiting examples of cardiovascular disease include atherosclerosis, myocardial infarction, myocardial ischemia, coronary artery stenosis (occlusion of the coronary arteries), chronic cardiovascular and/or arterial inflammation, acute cardiovascular and/or arterial inflammation, hypercholesterolemia, restenosis (narrowing of the vessel lumen), arrhythmia, thrombosis, hyperlipidemia, hypertension, dyslipoproteinemia, angina (cardiac chest pain), and vascular complications due to a cardiovascular disease (e.g., myocardial infarction or myocardial ischemia).

In some embodiments, at least one compound of Formula (I) and/or a pharmaceutical composition comprising at least one compound of Formula (I) may be administered prior to or subsequent to an acute cardiovascular event in the subject. In some embodiments, at least one compound of Formula (I) and/or a pharmaceutical composition comprising at least one compound of Formula (I) may be administered prior to or subsequent to the development or diagnosis of a cardiovascular disease, disorder and/or condition in the subject. In some embodiments, the acute cardiovascular event is a myocardial infarction.

In some embodiments, a method for treatment and/or prevention of atherosclerosis is disclosed, the method comprising administering to a subject in need thereof an effective amount of at least one compound of Formula (I) and/or a pharmaceutical composition comprising at least one compound of Formula (I). Atherosclerosis generally describes a disease of the arterial blood vessels. As used herein, “atherosclerosis” includes, but is not limited to, chronic and/or acute atherosclerotic inflammation prior to or subsequent to the formation of at least one atherosclerotic plaque in the subject. Atherosclerosis also includes, but is not limited to, chronic progressive atherosclerosis and/or atherosclerotic inflammation. Atherosclerosis also includes, but is not limited to, acute atherosclerosis and/or atherosclerotic inflammation subsequent to an acute vascular event in the subject (such as, for example, myocardial infarction).

In some embodiments, at least one compound of Formula (I) and/or a pharmaceutical composition comprising at least one compound of Formula (I) may be administered prior to or subsequent to the formation of at least one atherosclerotic plaque, lesion or atheroma in the subject.

In some embodiments, the formation, progression, destabilization and/or rupture of at least one atherosclerotic plaque within the subject is reduced.

Atherosclerotic plaques may be characterized as stable or unstable (i.e., vulnerable to destabilization). Unstable atherosclerotic plaques may be susceptible to disruption or rupture, which exposes thrombogenic material (i.e., thrombi) (e.g., collagen) to the circulation. This can produce interruptions in blood flood (ischemia) in local or distal arteries, which can result in cardiovascular complications, such as, for example, myocardial infarction (MI).

Destabilization of atherosclerotic plaques may occur via many mechanisms. Non-limiting examples of such mechanisms include superficial erosion of the endothelial cells that form the monolayer covering the intima, disruption of the microvessels that form in the atherosclerotic plaque, rupture (i.e., fracture) of the atherosclerotic plaque's fibrous cap, thinning or weakening of the fibrous cap (thus making it susceptible to rupture), and the presence or increase in inflammatory factors that mediate destabilization. (Libby P., Nature, 420; 868-874 (2002)).

A non-limiting example of inflammatory factors that mediate destabilization is the presence of inflammatory cells. The progression of atherosclerosis may be associated with systemically increased inflammatory myeloid cells that are recruited to atherosclerotic plaques. (Murphy, A. J. et al., J. Clin. Invest., 121; 4138-4149 (2011); Averill, L. E. et al., Am. J. Pathol., 135; 369-377 (1989); Feldman, D. L. et al., Arterioscler. Thromb., 11: 985-994 (1991); Swirski, F. K. et al., J. Clin. Invest., 117: 195-205 (2007)). The presence of inflammatory myeloid cells may be detrimental to a stable plaque. (Llodra, J. et al., Proc. Natl. Acad. Sci. U.S.A., 101: 11779-11784 (2004)).

In some embodiments, the stability of at least one atherosclerotic plaque within the subject is increased. Non-limiting examples of stable features of atherosclerotic plaques (i.e., stable phenotype) include smaller plaque size, reduced (i.e., decreased, diminished, smaller) necrotic core size (measured by, for example, necrotic core area), and a thicker fibrous cap of the atherosclerotic plaque (See, e.g., Moore K. J. et al., Cell, 145: 341-355 (2011)).

In some embodiments, the size of at least one atherosclerotic plaque within the subject is decreased. In some embodiments, the necrotic core size of at least one atherosclerotic plaque within the subject is decreased. In some embodiments, the fibrous cap thickness of at least one atherosclerotic plaque within the subject is increased.

In some embodiments, the administration of an effective amount of at least one compound of Formula (I) and/or a pharmaceutical composition comprising at least one compound of Formula (I) reduces the levels of extramedullary proliferation of hematopoietic stem and/or progenitor cells within the subject. In some embodiments, extramedullary proliferation of hematopoietic stem and/or progenitor cells is reduced in the spleen and/or the liver. Non-limiting examples of extramedullary proliferation of hematopoietic stem and/or progenitor cells include extramedullary hematopoiesis and extramedullary myelopoiesis.

In some embodiments, the administration of an effective amount of at least one compound of Formula (I) and/or a pharmaceutical composition comprising at least one compound of Formula (I) reduces the recruitment and/or infiltration of myeloid cells to at least one atherosclerotic plaque within the subject. Non-limiting examples of myeloid cells include monocytes, macrophages, neutrophils, basophils, eosinophils, erythrocytes, dendritic cells, and megakaryocytes and platelets.

In some embodiments, the at least one compound of Formula (I) and/or a pharmaceutical composition comprising at least one compound of Formula (I) is administered after angioplasty, stenting procedure, atherectomy, bypass surgery, or other vessel-corrective techniques.

In some embodiments, the at least one compound of Formula (I) and/or a pharmaceutical composition comprising at least one compound of Formula (I) is administered before angioplasty, stenting procedure, atherectomy, bypass surgery, or other vessel-corrective techniques.

In some embodiments, a method for treatment and prevention of myocardial infarction is disclosed, the method comprising administering to a subject in need thereof an effective amount of at least one compound of Formula (I) and/or a pharmaceutical composition comprising at least one compound of Formula (I). In some embodiments, the subject has previously suffered a myocardial infarction. In some embodiments, a compound of Formula (I) may be administered before the occurrence of a myocardial infarction in the subject. In some embodiments, a compound of Formula (I) may be administered after the occurrence of a first or subsequent myocardial infarction in the subject.

In some embodiments, at least one compound of Formula (I) and/or a pharmaceutical composition comprising at least one compound of Formula (I) is administered to the subject: within one (1) day of the subject suffering a myocardial infarction, within one (1) week of the subject suffering a myocardial infarction, within two (2) weeks of the subject suffering a myocardial infarction, within three (3) weeks of the subject suffering a myocardial infarction, within four (4) weeks of the subject suffering a myocardial infarction, within eight (8) weeks of the subject suffering a myocardial infarction, or within twelve (12) weeks of the subject suffering a myocardial infarction.

In some embodiments, a method for the treatment of sickle cell disease or complications associated therewith is disclosed, the method comprising administering to a subject in need thereof an effective amount of at least one compound of Formula (I) and/or a pharmaceutical composition comprising at least one compound of Formula (I).

In some embodiments, a method for treatment and prevention of vaso-occlusive crisis or complications associated therewith is disclosed, the method comprising administering to a subject in need thereof an effective amount of at least one compound of Formula (I) and/or a pharmaceutical composition comprising at least one compound of Formula (I).

In some embodiments, the pathological angiogenesis in the eye. Examples of ocular diseases, disorders, or conditions associated with pathological angiogenesis include age-related macular degeneration, ocular histoplasmosis syndrome, neovascular glaucoma, retrolental fibroplasia, pathologic myopia, angioid streaks, idiopathic disorders, choroiditis, choroidal rupture, overlying choroid nevi, graft rejection, herpes simplex keratitis, leishmaniasis, onchocerciasis, certain inflammatory diseases such as dry eye syndrome, and trauma to the eye (e.g., cornea).

In some embodiments, the present disclosure is directed to methods for treatment and prevention of pathological angiogenesis in patients with cancer.

The compounds of the present disclosure and pharmaceutical compositions comprising at least one such compound may be administered in combination with at least one additional antiepileptic agent (e.g. acetazolamide, carbamazepine, clobazam, clonazepam, eslicarbazepine acetate, ethosuximide, gabapentin, lacosamide, lamotrigine, levetiracetam, nitrazepam, oxcarbazepine, perampanel, piracetam, phenobarbital, phenytoin, pregabalin, primidone, rufinamide, sodium valproate, stiripentol, tiagabine, topiramate, vigabatrin, zonisamide). The compounds of the present disclosure and pharmaceutical compositions comprising at least one such compound may function independently from the antiepileptic agent or may function in coordination with the at least one antiepileptic agent. In addition, the administration of one or more of the compounds or compositions may be in conjunction with one or more other therapies, e.g., for reducing toxicities of therapy. For example, at least one palliative agent to counteract (at least in part) a side effect of therapy may be administered. Agents (chemical or biological) that promote recovery or enhancement of appetite, or counteract nausea or fatigue, are examples of such agents. The compounds of the present disclosure and pharmaceutical composition comprising at least one such compound may be administered before, after, or concurrently with administration of at least one additional anti-thrombosis agent or at least one palliative agent to reduce a side effect of therapy. Where administration is concurrent, the combination may be administered from a single container or two (or more) separate containers.

The terms, “treat” and “treatment,” include medical management of a disease, disorder, and/or condition of a subject as would be understood by a person of ordinary skill in the art (see, e.g., Stedman's Medical Dictionary). In general, an appropriate dose and treatment regimen provide at least one of the compounds of the present disclosure in an amount sufficient to provide therapeutic and/or prophylactic benefit. For both therapeutic treatment and prophylactic or preventative measures, therapeutic and/or prophylactic benefit includes, for example, an improved clinical outcome, wherein the object is to prevent or slow or lessen an undesired physiological change or disorder, or to prevent or slow or lessen the expansion or severity of such disorder. As discussed herein, beneficial or desired clinical results from treating a subject include, but are not limited to, abatement, lessening, or alleviation of symptoms that result from or are associated with the disease, condition, and/or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease: delay or slowing of disease progression; amelioration or palliation of the disease state; and remission (whether partial or total), whether detectable or undetectable; and/or overall survival. “Treatment” can include prolonging survival when compared to expected survival if a subject were not receiving treatment.

In some embodiments of the methods described herein, the subject is a human. In some embodiments of the methods described herein, the subject is a non-human animal. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

The effectiveness of the compounds of the present disclosure in treating and/or preventing diseases, disorders, and/or conditions treatable by inhibiting an activity of E-selectin, galectin-3, and/or CXCR4 chemokine receptors can readily be determined by a person of ordinary skill in the relevant art. Determining and adjusting an appropriate dosing regimen (e.g., adjusting the amount of compound per dose and/or number of doses and frequency of dosing) can also readily be performed by a person of ordinary skill in the relevant art. One or any combination of diagnostic methods, including physical examination, assessment and monitoring of clinical symptoms, and performance of analytical tests and methods described herein, may be used for monitoring the health status of the subject.

Also provided herein are pharmaceutical compositions comprising at least one compound of Formula (I). In some embodiments, the pharmaceutical compositions further comprise at least one additional pharmaceutically acceptable ingredient.

In pharmaceutical compositions, any one or more of the compounds of the present disclosure may be administered in the form of a pharmaceutically acceptable derivative, such as a salt, and/or it or they may also be used alone and/or in appropriate association, as well as in combination, with other pharmaceutically active compounds.

An effective amount or therapeutically effective amount refers to an amount of at least one compound of the present disclosure or a pharmaceutical composition comprising at least one such compound that, when administered to a subject, either as a single dose or as part of a series of doses, is effective to produce at least one therapeutic effect. Optimal doses may generally be determined using experimental models and/or clinical trials. Design and execution of pre-clinical and clinical studies for each of the therapeutics (including when administered for prophylactic benefit) described herein are well within the skill of a person of ordinary skill in the relevant art. The optimal dose of a therapeutic may depend upon the body mass, weight, and/or blood volume of the subject.

In general, the amount of at least one compound of Formula (I) as described herein, that is present in a dose, may range from about 0.01 μg to about 100 mg per kg weight of the subject. In some embodiments, the amount of at least one compound of Formula (I) that is present in a dose may range from about 0.01 μg to about 40 mg per kg weight of the subject. In some embodiments, the amount of at least one compound of Formula (I) that is present in a dose may range from about 0.01 μg to about 20 mg per kg weight of the subject. In some embodiments, the amount of at least one compound of Formula (I) that is present in a dose may range from about 0.1 mg to about 100 mg per kg weight of the subject. In some embodiments, the amount of at least one compound of Formula (I) that is present in a dose may range from about 0.1 mg to about 40 mg per kg weight of the subject. In some embodiments, the amount of at least one compound of Formula (I) that is present in a dose may range from about 0.1 mg to about 20 mg per kg weight of the subject.

The minimum dose that is sufficient to provide effective therapy may be used in some embodiments. Subjects may generally be monitored for therapeutic effectiveness using assays suitable for the disease, disorder and/or condition being treated or prevented, which assays will be familiar to those having ordinary skill in the art and are described herein. The level of a compound that is administered to a subject may be monitored by determining the level of the compound (or a metabolite of the compound) in a biological fluid, for example, in the blood, blood fraction (e.g., serum), and/or in the urine, and/or other biological sample from the subject. Any method practiced in the art to detect the compound, or metabolite thereof, may be used to measure the level of the compound during the course of a therapeutic regimen.

The dose of a compound described herein may depend upon the subject's condition, that is, stage of the disease, severity of symptoms caused by the disease, general health status, as well as age, gender, and weight, and other factors apparent to a person of ordinary skill in the medical art. Similarly, the dose of the therapeutic for treating a disease, disorder, and/or condition may be determined according to parameters understood by a person of ordinary skill in the medical art.

Pharmaceutical compositions may be administered in any manner appropriate to the disease, disorder, and/or condition to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the composition(s) as described herein in an amount sufficient to provide therapeutic and/or prophylactic benefit (for example, an improved clinical outcome, such as more frequent complete or partial remissions, or longer disease-free and/or overall survival, or a lessening of symptom severity or other benefit as described in detail above).

The pharmaceutical compositions described herein may be administered to a subject in need thereof by any one of several routes that effectively delivers an effective amount of the compound. Non-limiting examples of suitable administrative routes include topical, oral, nasal, intrathecal, enteral, buccal, sublingual, transdermal, rectal, vaginal, intraocular, subconjunctival, sublingual, and parenteral administration, including subcutaneous, intravenous, intramuscular, intrasternal, intracavernous, intrameatal, and intraurethral injection and/or infusion.

The pharmaceutical compositions described herein may, for example, be sterile aqueous or sterile non-aqueous solutions, suspensions, or emulsions, and may additionally comprise at least one pharmaceutically acceptable excipient (i.e., a non-toxic material that does not interfere with the activity of the active ingredient). Such compositions may, for example, be in the form of a solid, liquid, or gas (aerosol). Alternatively, the compositions described herein may, for example, be formulated as a lyophilizate, or compounds described herein may be encapsulated within liposomes using technology known in the art. The pharmaceutical compositions may further comprise at least one additional pharmaceutically acceptable ingredient, which may be biologically active or inactive. Non-limiting examples of such ingredients include buffers (e.g., neutral buffered saline or phosphate buffered saline), carbohydrates (e.g., glucose, mannose, sucrose or dextrans), mannitol, proteins, polypeptides, amino acids (e.g., glycine), antioxidants, chelating agents (e.g., EDTA and glutathione), stabilizers, dyes, flavoring agents, suspending agents, and preservatives.

Any suitable excipient or carrier known to those of ordinary skill in the art for use in compositions may be employed in the compositions described herein. Excipients for therapeutic use are well known, and are described, for example, in Remington: The Science and Practice of Pharmacy (Gennaro, 21^{st} Ed. Mack Pub. Co., Easton, Pa. (2005)). In general, the type of excipient may be selected based on the mode of administration, as well as the chemical composition of the active ingredient(s). Compositions may be formulated for the particular mode of administration. For parenteral administration, pharmaceutical compositions may further comprise water, saline, alcohols, fats, waxes, and buffers. For oral administration, pharmaceutical compositions may further comprise at least one component chosen, for example, from any of the aforementioned ingredients, excipients and carriers, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose, and magnesium carbonate.

The pharmaceutical compositions (e.g., for oral administration or delivery by injection) may be in the form of a liquid. A liquid composition may include, for example, at least one the following: a sterile diluent such as water for injection, saline solution, including for example physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. In some embodiments, the pharmaceutical composition comprises physiological saline. In some embodiments, the pharmaceutical composition is an injectable composition, and in some embodiments, the injectable composition is sterile.

For oral formulations, at least one of the compounds of the present disclosure can be used alone or in combination with at least one additive appropriate to make tablets, powders, granules and/or capsules, for example, those chosen from conventional additives, disintegrators, lubricants, diluents, buffering agents, moistening agents, preservatives, coloring agents, and flavoring agents. The pharmaceutical compositions may be formulated to include at least one buffering agent, which may provide for protection of the active ingredient from low pH of the gastric environment and/or an enteric coating. A pharmaceutical composition may be formulated for oral delivery with at least one flavoring agent, e.g., in a liquid, solid or semi-solid formulation and/or with an enteric coating.

Oral formulations may be provided as gelatin capsules, which may contain the active compound or biological along with powdered carriers. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract.

A pharmaceutical composition may be formulated for sustained or slow release. Such compositions may generally be prepared using well known technology and administered by, for example, oral, rectal or subcutaneous implantation, or by implantation at the desired target site. Sustained-release formulations may contain the active therapeutic dispersed in a carrier matrix and/or contained within a reservoir surrounded by a rate controlling membrane. Excipients for use within such formulations are biocompatible, and may also be biodegradable; the formulation may provide a relatively constant level of active component release. The amount of active therapeutic contained within a sustained release formulation depends upon the site of implantation, the rate and expected duration of release, and the nature of the condition to be treated or prevented.

The pharmaceutical compositions described herein can be formulated as suppositories by mixing with a variety of bases such as emulsifying bases or water-soluble bases. The pharmaceutical compositions may be prepared as aerosol formulations to be administered via inhalation. The pharmaceutical compositions may be formulated into pressurized acceptable propellants such as dichlorodifluoromethane, propane, nitrogen and the like.

The compounds of the present disclosure and pharmaceutical compositions comprising these compounds may be administered topically (e.g., by transdermal administration). Topical formulations may be in the form of a transdermal patch, ointment, paste, lotion, cream, gel, and the like. Topical formulations may include one or more of a penetrating agent or enhancer (also call permeation enhancer), thickener, diluent, emulsifier, dispersing aid, or binder. Physical penetration enhancers include, for example, electrophoretic techniques such as iontophoresis, use of ultrasound (or “phonophoresis”), and the like. Chemical penetration enhancers are agents administered either prior to, with, or immediately following administration of the therapeutic, which increase the permeability of the skin, particularly the stratum corneum, to provide for enhanced penetration of the drug through the skin. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995): Lenneräs et al., J. Pharm. Pharmacol. 54:499-508 (2002); Karande et al., Pharm. Res. 19:655-60 (2002); Vaddi et al., Int. J. Pharm. 91:1639-51 (2002); Ventura et al., J. Drug Target 9:379-93 (2001); Shokri et al., Int. J. Pharm. 228(1-2):99-107 (2001): Suzuki et al., Biol. Pharm. Bull. 24:698-700 (2001); Alberti et al., J. Control Release 71:319-27 (2001); Goldstein et al., Urology 57:301-5 (2001); Kiijavainen et al., Eur. J. Pharm. Sci. 10:97-102 (2000); and Tenjarla et al., Int. J. Pharm. 192:147-58 (1999).

Kits comprising unit doses of at least one compound of the present disclosure, for example in oral or injectable doses, are provided. Such kits may include a container comprising the unit dose, an informational package insert describing the use and attendant benefits of the therapeutic in treating the pathological condition of interest, and/or optionally an appliance or device for delivery of the at least one compound of Formula (I) and/or pharmaceutical composition comprising the same.

### EXAMPLES

### Example 1

### Prophetic Synthesis of Multimeric Compound 21

Compound 3: A mixture of compounds 1 (preparation described in WO 2007/028050) and compound 2 (preparation described in WO 2013/096926) (1.7 eq) is azeotroped 3 times from toluene. The mixture is dissolved in DCM under argon and cooled on an ice bath. To this solution is added boron trifluoride etherate (1.5 eq). The reaction mixture is stirred 12 hours at room temperature. The reaction is quenched by the addition of triethylamine (2 eq). The reaction mixture is transferred to a separatory funnel and washed 1 time with half saturated sodium bicarbonate solution and 1 time with water. The organic phase is dried over sodium sulfate, filtered, and concentrated. The residue is purified by flash chromatography to afford compound 3.

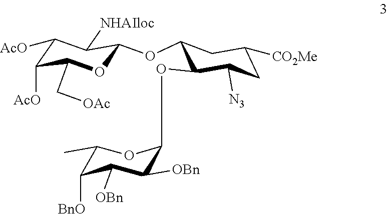

Compound 4: Compound 3 is dissolved in methanol at room temperature. A solution of sodium methoxide in methanol (0.1 eq) is added and the reaction mixture stirred overnight at room temperature. The reaction mixture is quenched by the addition of acetic acid. The reaction mixture is diluted with ethyl acetate, transferred to a separatory funnel and washed 2 times with water. The organic phase is dried over magnesium sulfate, filtered and concentrated. The residue is separated by flash chromatography to afford compound 4.

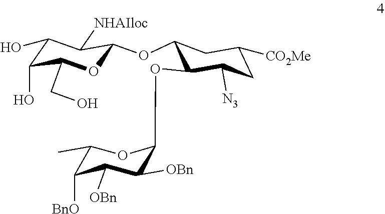

Compound 5: To a solution of compound 4 in dichloromethane cooled on an ice bath is added DABCO (1.5 eq) followed by monomethyoxytrityl chloride (1.2 eq). The reaction mixture is stirred overnight allowing to warm to room temperature. The reaction mixture is transferred to a separatory funnel and washed 2 times with water. The organic phase is concentrated and the residue is purified by flash chromatography to afford compound 5.

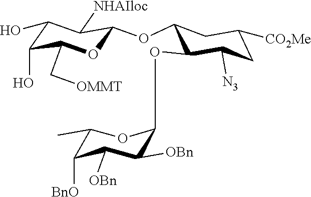

Compound 7: To a solution of compound 5 in methanol is added dibutyltin oxide (1.1 eq). The reaction mixture is refluxed for 3 hours then concentrated. The residue is suspended in DME. To this suspension is added compound 6 (preparation described in Thoma et. al. J. Med. Chem., 1999, 42, 4909) (1.5 eq) followed by cesium fluoride (1.2 eq). The reaction mixture is stirred at room temperature overnight. The reaction mixture is diluted with ethyl acetate, transferred to a separatory funnel, and washed with water. The organic phase is dried over sodium sulfate, filtered and concentrated. The residue is purified by flash chromatography to afford compound 7.

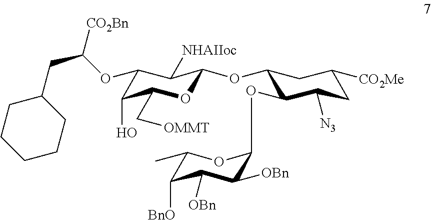

Compound 8: To a degassed solution of compound 7 in anhydrous DCM at 0° C. is added Pd(PPh₃)₄ (0.1 eq), Bu₃SnH (1.1 eq) and N-trifluoroacetyl glycine anhydride (2.0 eq) (preparation described in Chemische Berichle (1955), 88(1), 26). The resulting solution is stirred for 12 hrs allowing the temperature to increase to room temperature. The reaction mixture is diluted with DCM, transferred to a separatory funnel, and washed with water. The organic phase is dried over Na₂SO₄, then filtered and concentrated. The residue is purified by flash chromatography to afford compound 8.

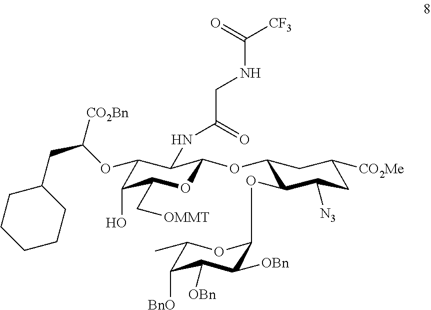

Compound 9: To a stirred solution of compound 8 in DCM/MeOH (25/1) at room temperature is added orotic acid chloride (5 eq) and triphenylphosphine (5 eq). The reaction mixture is stirred 24 hours. The solvent is removed and the residue is separated by column chromatography to afford compound 9.

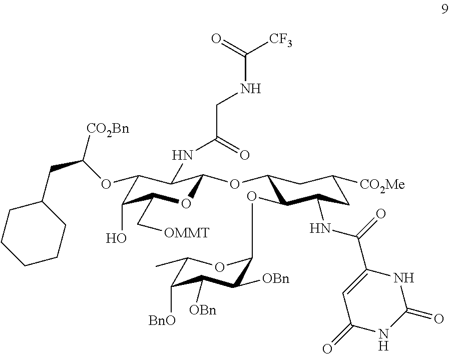

Compound 10: Compound 9 is dissolved in methanol and degassed. To this solution is added Pd(OH)₂/C. The reaction mixture is vigorously stirred under a hydrogen atmosphere for 12 hours. The reaction mixture is filtered through a Celite pad. The filtrate is concentrated under reduced pressure to give compound 10.

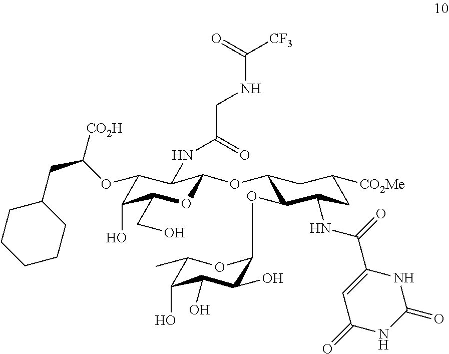

Compound 11: Compound 10 is dissolved in methanol at room temperature. A solution of sodium methoxide in methanol (1.1 eq) is added and the reaction mixture stirred overnight at room temperature. The reaction mixture is quenched by the addition of acetic acid. The reaction mixture is concentrated. The residue is separated by C-18 reverse phase chromatography to afford compound 11.

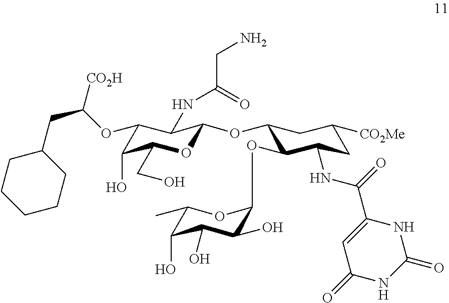

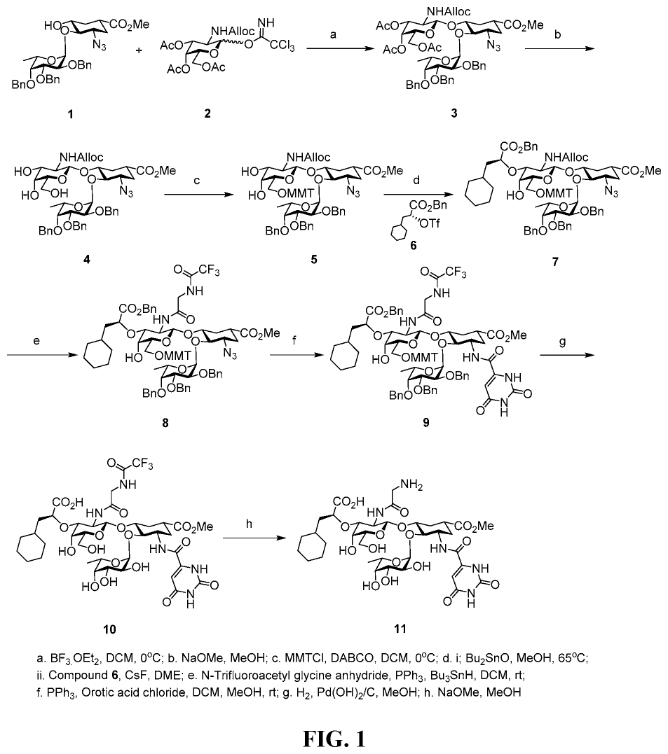

Compound 12: Compound 12 can be prepared in an analogous fashion to FIG. 1 by substituting (acetylthio)acetyl chloride for N-trifluoroacetyl glycine anhydride in step e.

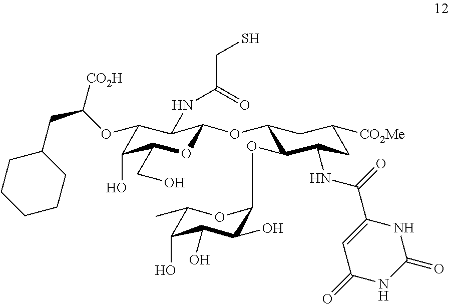

Compound 13: Compound 10 is dissolved in DMF and cooled on an ice bath. Diisopropylethylamine (1.5 eq) is added followed by HATU (1.1 eq). The reaction mixture is stirred 15 minutes on the ice bath then azetidine (2 eq) is added. The ice bath is removed and the reaction mixture is stirred overnight at room temperature. The solvent is removed under reduced pressure and the residue is separated by flash chromatography to afford compound 13.

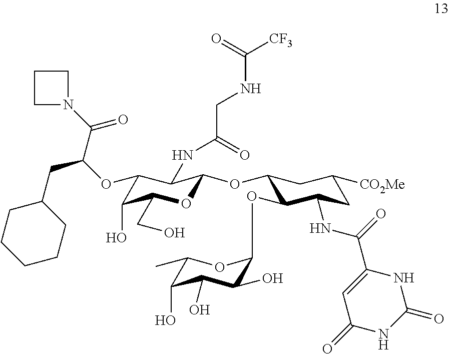

Compound 14: Compound 13 is dissolved in methanol at room temperature. A solution of sodium methoxide in methanol (0.3 eq) is added and the reaction mixture stirred overnight at room temperature. The reaction mixture is quenched by the addition of acetic acid. The reaction mixture is concentrated. The residue is separated by C-18 reverse phase chromatography to afford compound 14.

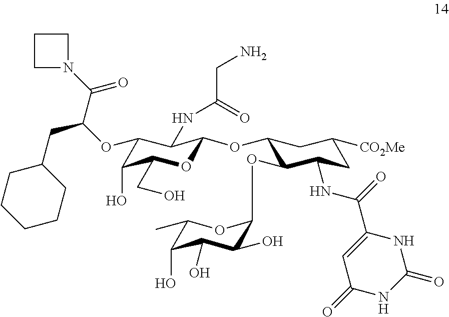

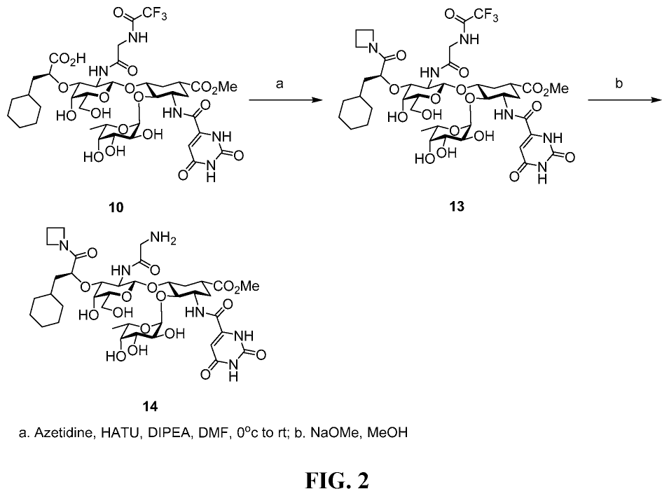

Compound 15: Compound 15 can be prepared in an analogous fashion to FIG. 2 by using methylamine in place of azetidine in step a.

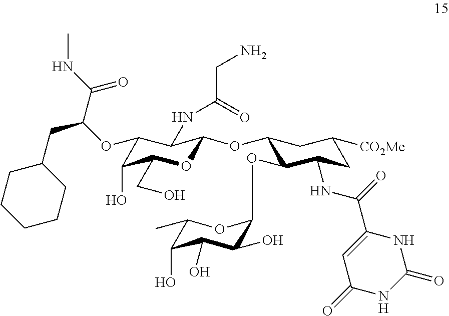

Compound 16: Compound 16 can be prepared in an analogous fashion to FIG. 2 by using dimethylamine in place of azetidine in step a.

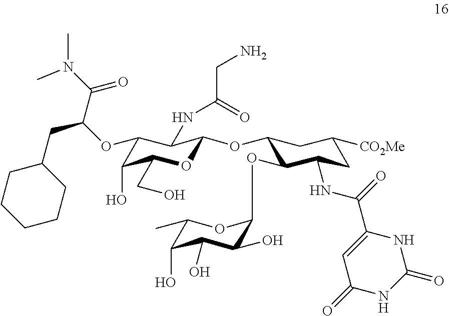

Compound 17: Compound 17 can be prepared in an analogous fashion to FIG. 2 by using 2-methoxyethylamine in place of azetidine in step a.

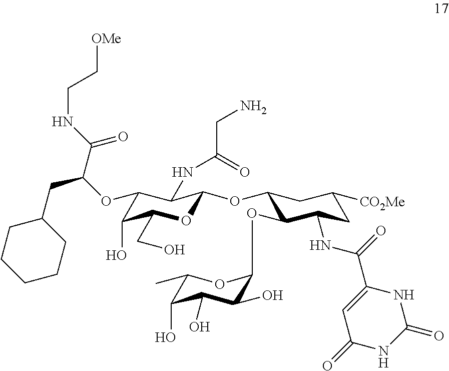

Compound 18: Compound 18 can be prepared in an analogous fashion to FIG. 2 by using piperidine in place of azetidine in step a.

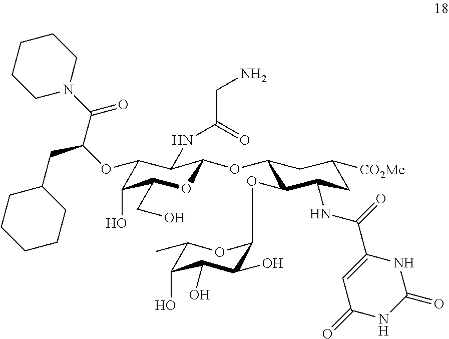

Compound 19: Compound 19 can be prepared in an analogous fashion to FIG. 2 by using morpholine in place of azetidine in step a.

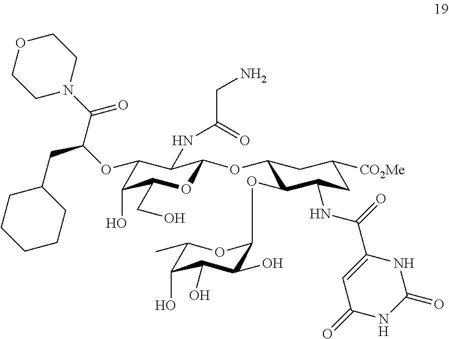

Compound 21: A solution of compound 20 (0.4 eq) in DMSO is added to a solution of compound 11 (1 eq) and DIPEA (10 eq) in anhydrous DMSO at room temperature. The resulting solution is stirred overnight. The solution is dialyzed against distilled water for 3 days with dialysis tube MWCO 1000 while distilled water is changed every 12 hours. The solution in the tube is lyophilized to give compound 21.

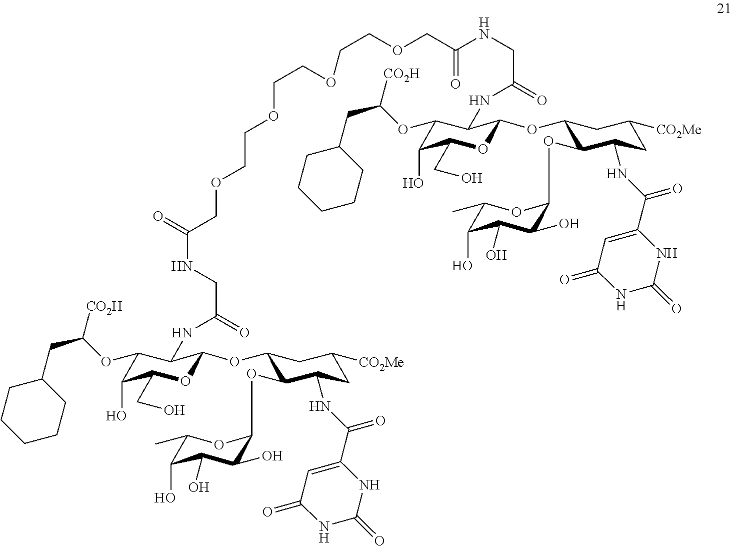

### Example 2

### Prophetic Synthesis of Multimeric Compound 22

Compound 22: A solution of compound 21 in ethylenediamine is stirred overnight at 70° C. The reaction mixture is concentrated under reduced pressure and the residue is purified by reverse phase chromatography to give compound 22.

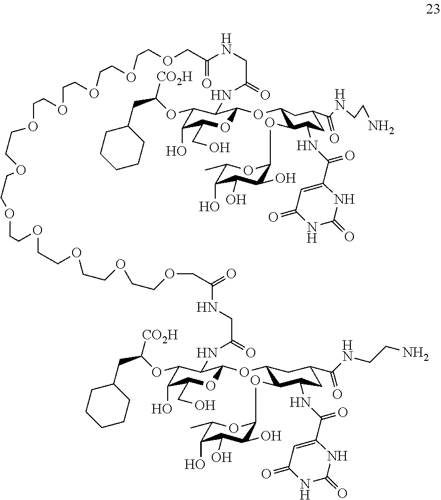

### Example 3

### Prophetic Synthesis of Multimeric Compound 23

Compound 23: Compound 23 can be prepared in an analogous fashion to FIG. 3 by replacing compound 20 with PEG-11 diacetic acid di-NHS ester in step a.

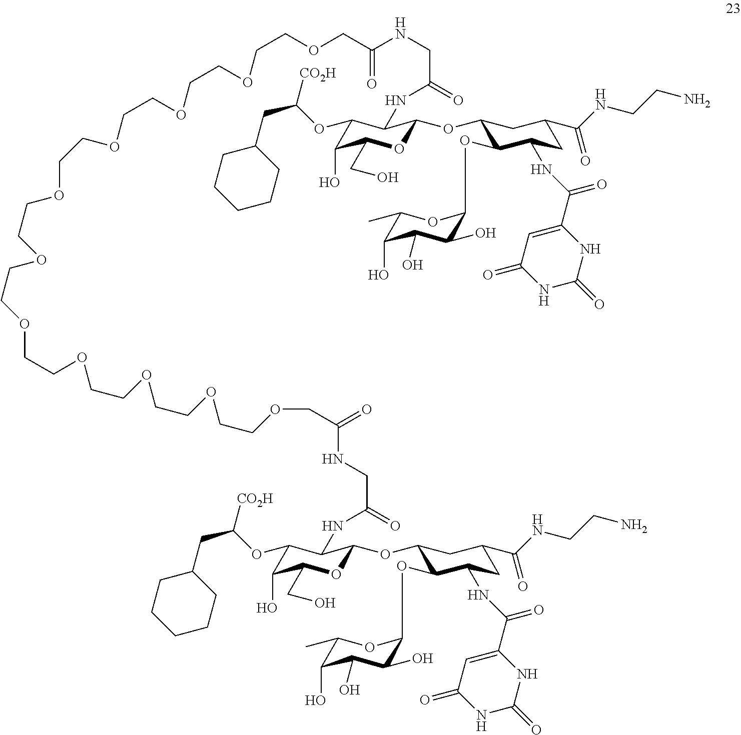

### Example 4

### Prophetic Synthesis of Multimeric Compound 24

Compound 24: Compound 24 can be prepared in an analogous fashion to FIG. 3 by replacing compound 20 with PEG-15 diacetic acid di-NHS ester in step a.

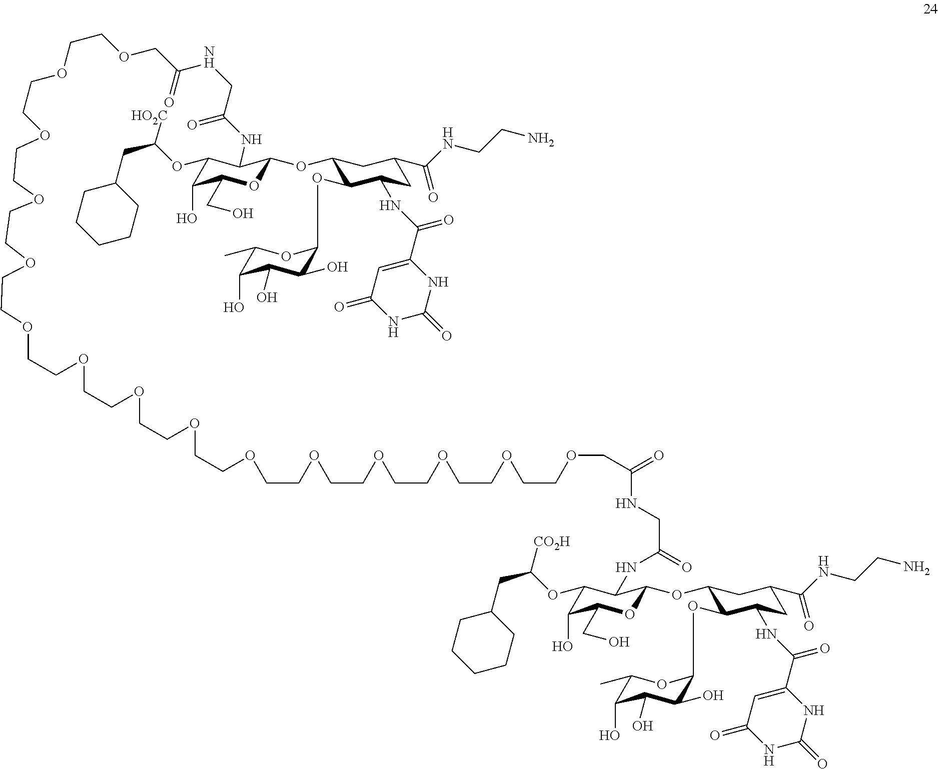

### Example 5

### Prophetic Synthesis of Multimeric Compound 25

Compound 25: Compound 25 can be prepared in an analogous fashion to FIG. 3 by replacing compound 20 with ethylene glycol diacetic acid di-NHS ester in step a.

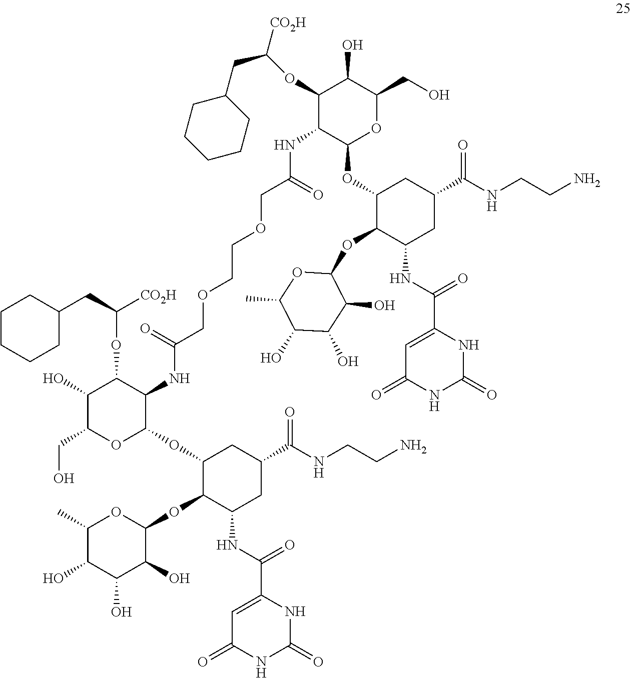 [SMILES: C[C@@H]1O[C@@H](O[C@@H]2[C@@H](NC(=O)c3cc(=O)[nH]c(=O)[nH]3)C[C@@H](C(=O)NCCN)C[C@H]2O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3C[C@H](C(=O)NCCN)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H](O)[C@H](O)[C@@H]1O]

### Example 6

### Prophetic Synthesis of Multimeric Compound 26

Compound 26: Compound 26 can be prepared in an analogous fashion to FIG. 3 by replacing compound 20 with 3,3′-[[2,2-bis[[3-[(2,5-dioxo-1-pyrrolidinyl)oxy]-3-oxopropoxy]methyl]-1,3-propanediyl]bis(oxy)]bis-1,1′-bis(2,5-dioxo-1-pyrrolidinyl)-propanoic acid ester in step a.

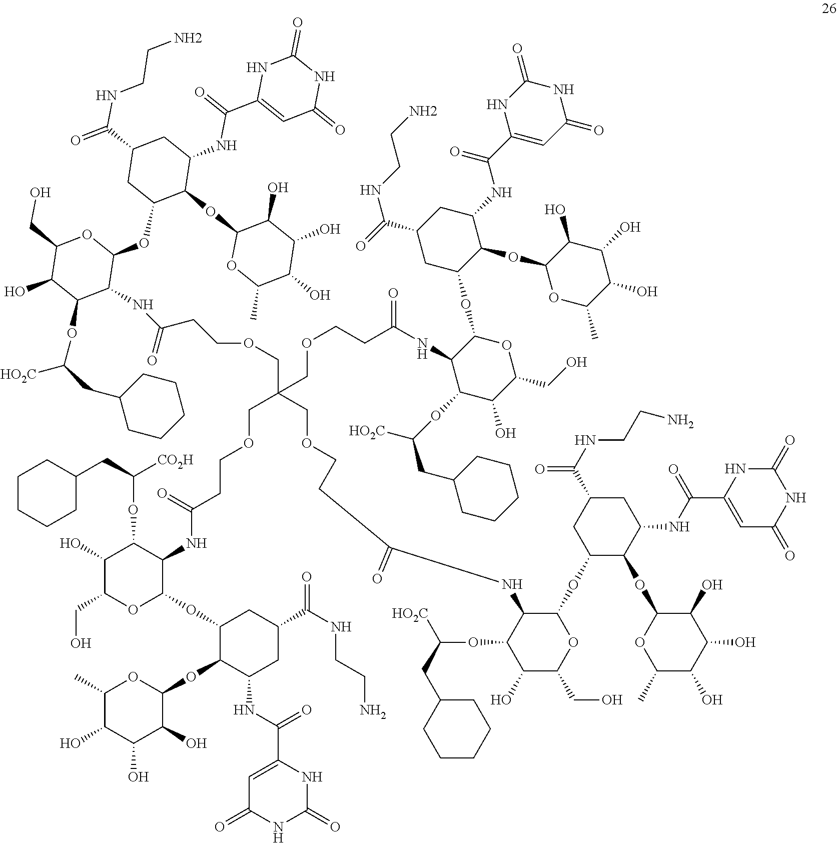

### Example 7

### Prophetic Synthesis of Multimeric Compound 27

Compound 27: Compound 27 can be prepared in an analogous fashion to FIG. 3 by replacing ethylenediamine with 2-aminoethyl ether in step b.

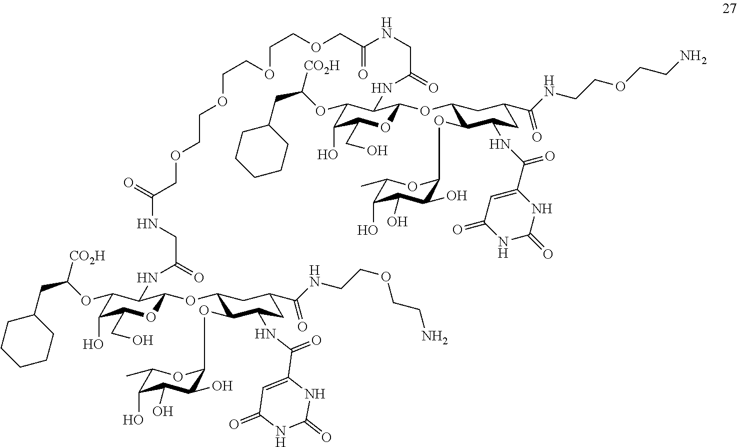

### Example 8

### Prophetic Synthesis of Multimeric Compound 28

Compound 28: Compound 28 can be prepared in an analogous fashion to FIG. 3 by replacing ethylenediamine with 1,5-diaminopentane in step b.

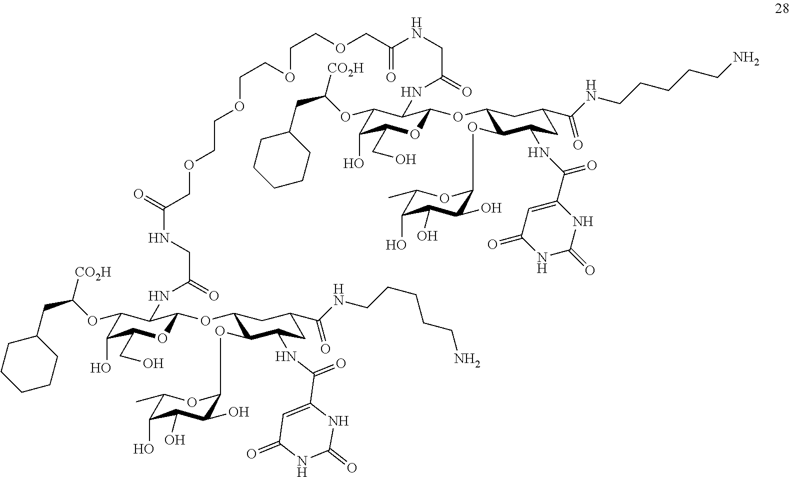

### Example 9

### Prophetic Synthesis of Multimeric Compound 29

Compound 29: Compound 29 can be prepared in an analogous fashion to FIG. 3 by replacing ethylenediamine with 1,2-bis(2-aminoethoxy)ethane in step b.

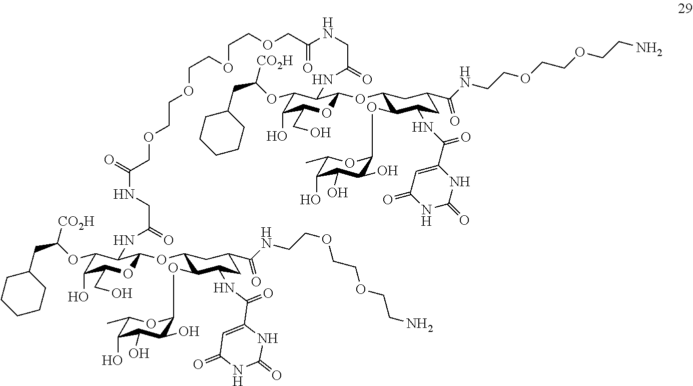

### Example 10

### Prophetic Synthesis of Multimeric Compound 30

Compound 30: Compound 30 can be prepared in an analogous fashion to FIG. 3 by replacing compound 11 with compound 14 and compound 20 with PEG-11 diacetic acid di-NHS ester in step a.

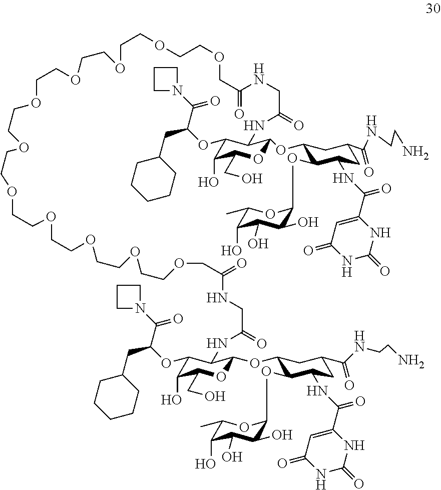

### Example 11

### Prophetic Synthesis of Multimeric Compound 31

Compound 31: Compound 31 can be prepared in an analogous fashion to FIG. 3 by replacing compound 11 with compound 15 in step a.

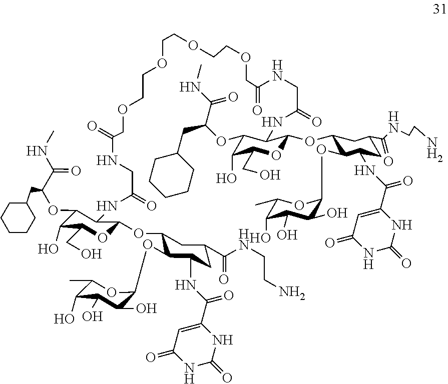

### Example 12

### Prophetic Synthesis of Multimeric Compound 32

Compound 32: Compound 32 can be prepared in an analogous fashion to FIG. 3 by replacing compound 11 with compound 17 and compound 20 with PEG-15 diacetic acid di-NHS ester in step a.

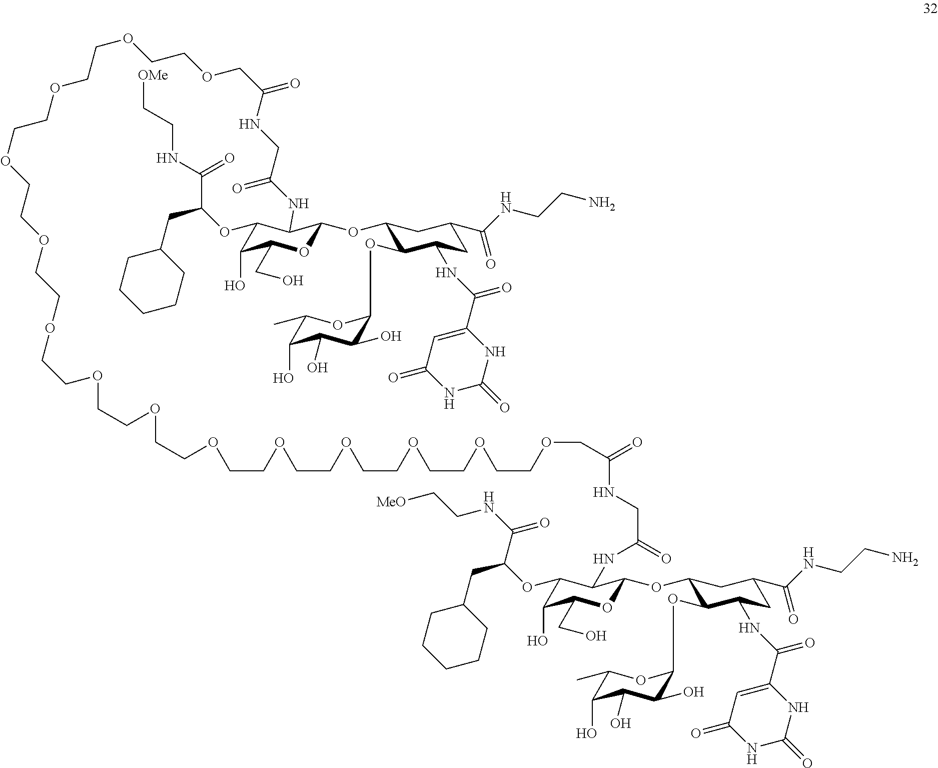

### Example 13

### Prophetic Synthesis of Multimeric Compound 33

Compound 33: Compound 33 can be prepared in an analogous fashion to FIG. 3 by replacing compound 11 with compound 16 and compound 20 with ethylene glycol diacetic acid di-NHS ester in step a.

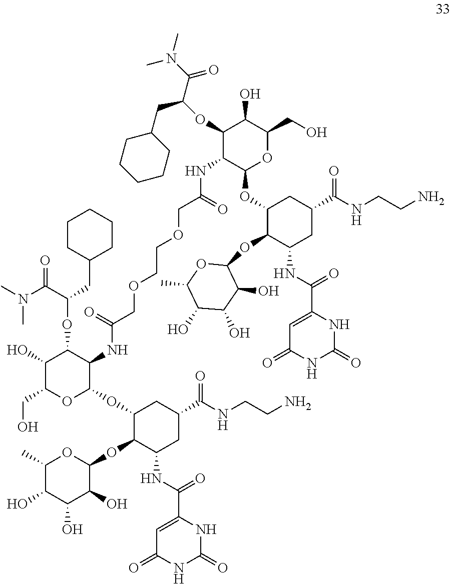 [SMILES: C[C@@H]1O[C@@H](O[C@@H]2[C@@H](NC(=O)c3cc(=O)[nH]c(=O)[nH]3)C[C@@H](C(=O)NCCN)C[C@H]2O[C@@H]2O[C@H](CO)[C@H](O)[C@H](O[C@@H](CC3CCCCC3)C(=O)N(C)C)[C@H]2NC(=O)COCCOCC(=O)N[C@H]2[C@H](O[C@@H]3C[C@H](C(=O)NCCN)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N(C)C)[C@@H](O)[C@H](O)[C@@H]1O]

### Example 14

### Prophetic Synthesis of Multimeric Compound 24

Compound 34: Compound 34 can be prepared in an analogous fashion to FIG. 3 by replacing compound 11 with compound 18 in step a and replacing ethylenediamine with 2-aminoethyl ether in step b.

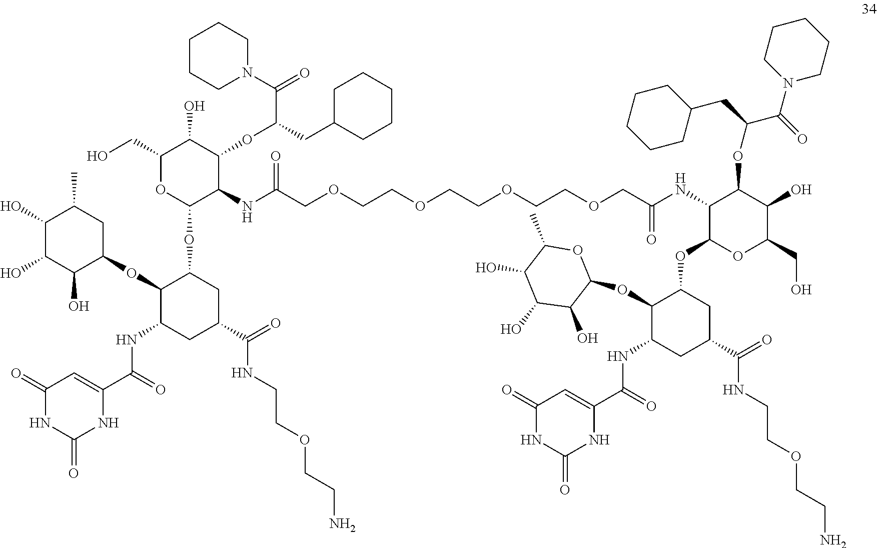 [SMILES: C[C@@H]1C[C@@H](O[C@@H]2[C@@H](NC(=O)c3cc(=O)[nH]c(=O)[nH]3)C[C@@H](C(=O)NCCOCCN)C[C@H]2O[C@@H]2O[C@H](CO)[C@H](O)[C@H](O[C@@H](CC3CCCCC3)C(=O)N3CCCCC3)[C@H]2NC(=O)COCCOCCOCCOCC(=O)N[C@H]2[C@H](O[C@@H]3C[C@H](C(=O)NCCOCCN)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCCCC2)[C@@H](O)[C@H](O)[C@@H]1O]

### Example 15

### Prophetic Synthesis of Multimeric Compound 36

Compound 36: To a solution of compound 12 in MeOH at room temperature is added compound 35 followed by cesium acetate (2.5 eq). The reaction mixture is stirred at room temperature until completion. The solvent is removed under reduced pressure. The product is purified by reverse phase chromatography to give compound 36.

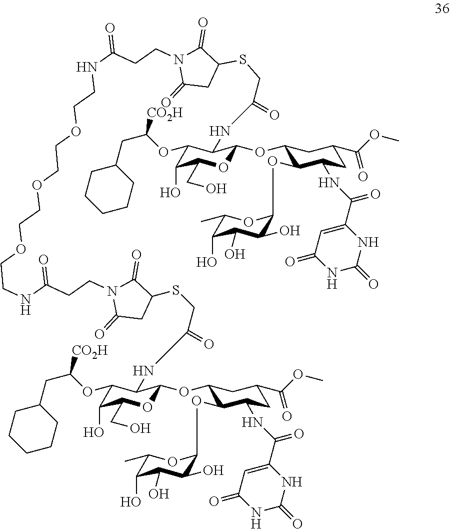

### Example 16

### Prophetic Synthesis of Multimeric Compound 37

Compound 37: Compound 36 is dissolved in ethylenediamine and the reaction mixture is stirred overnight at 70° C. The reaction mixture is concentrated under reduced pressure and the residue is purified by reverse phase chromatography to give compound 37.

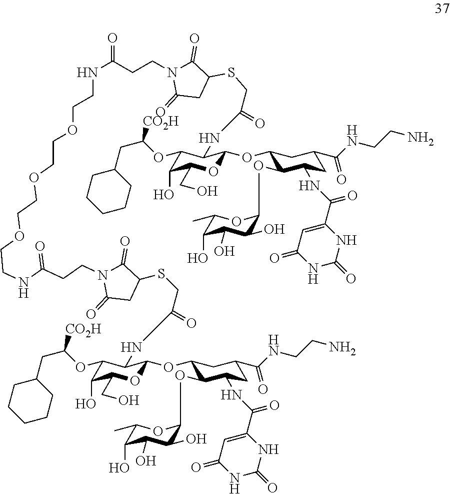

### Example 17

### Prophetic Synthesis of Multimeric Compound 38

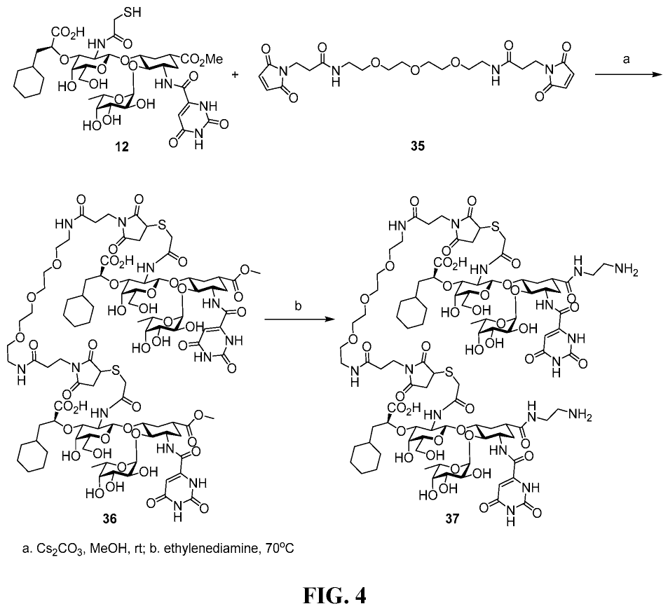

Compound 38: Compound 38 can be prepared in an analogous fashion to FIG. 4 by substituting PEG-6-bis maleimidylpropionamide for compound 35 in step a.

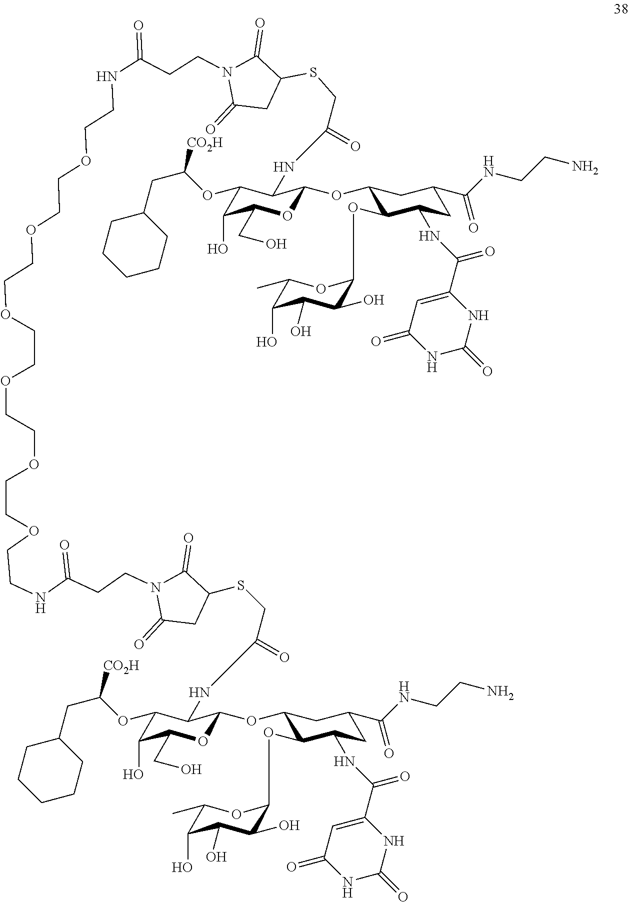

### Example 18

### Prophetic Synthesis of Multimeric Compound 39

Compound 39: Compound 39 can be prepared in an analogous fashion to FIG. 4 by substituting compound 35 for, 1,1′-[[2,2-bis[[3-(2,5-dihydro-2,5-dioxo-1H-pyrrol-1-yl) propoxy]methyl]-1,3-propanediyl]bis(oxy-3,1-propanediyl)]bis-1H-pyrrole-2,5-dione in step a.

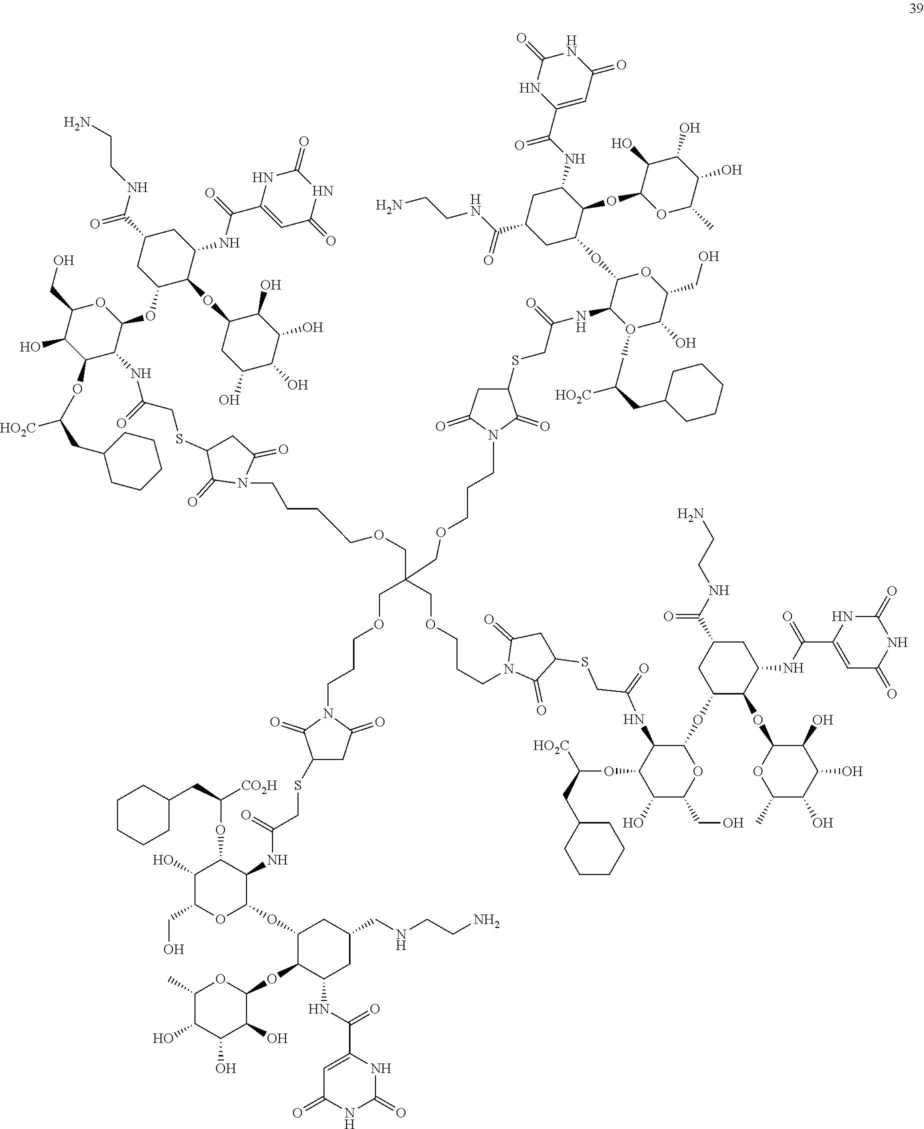

### Example 19

### Prophetic Synthesis of Multimeric Compound 40

Compound 40: Compound 40 can be prepared in an analogous fashion to FIG. 4 by substituting propylenediamine for ethylenediamine in step b.

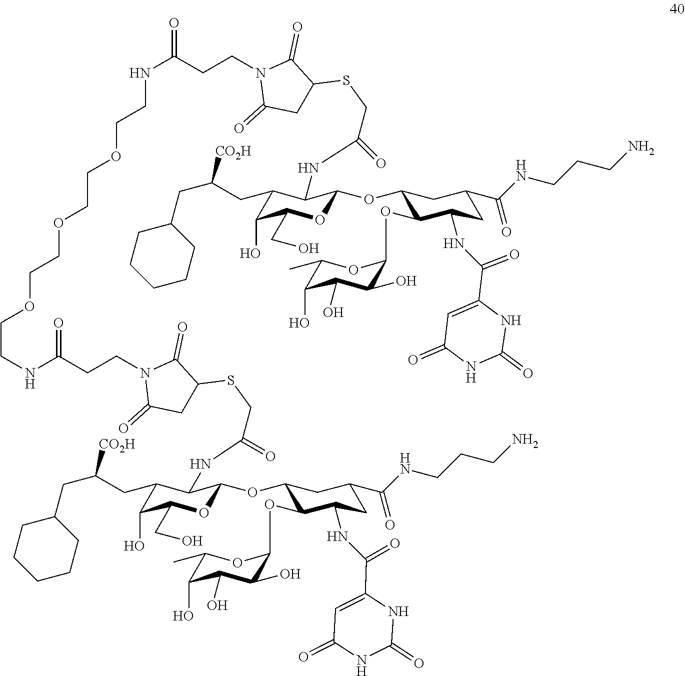

### Example 20

### Prophetic Synthesis of Multimeric Compound 44

Compound 41: To a stirred solution of compound 7 in DCM/MeOH (25/1) at room temperature is added orotic acid chloride (5 eq) and triphenylphosphine (5 eq). The reaction mixture is stirred 24 hours. The solvent is removed and the residue is separated by column chromatography to afford compound 41.

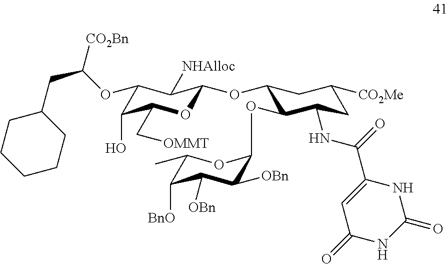

Compound 42: To a degassed solution of compound 41 in anhydrous DCM at 0° C. is added Pd(PPh₃)₄ (0.1 eq), Bu₃SnH (1.1 eq) and azidoacetic anhydride (2.0 eq). The ice bath is removed and the solution is stirred for 12 hrs under a N₂ atmosphere at room temperature. The reaction mixture is diluted with DCM, washed with water, dried over Na₂SO₄, then concentrated. The crude product is purified by column chromatography to give compound 42.

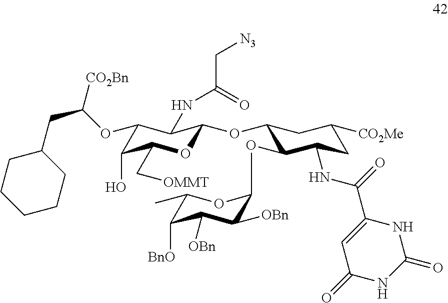

Compound 44: A solution of bispropagyl PEG-5 (compound 43) and compound 42 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred 12 hrs at 70° C. The solution is cooled to room temperature and concentrated under reduced pressure. The crude product is purified by chromatography to give compound 44.

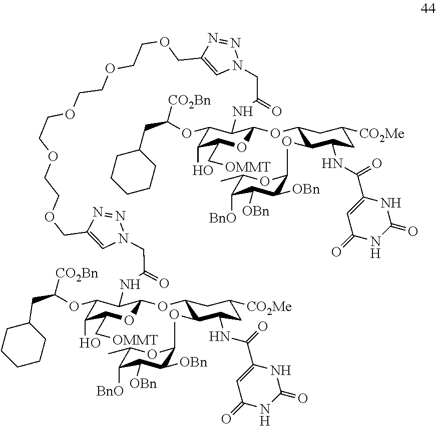

### Example 21

### Prophetic Synthesis of Multimeric Compound 45

Compound 45: Compound 44 is dissolved in MeOH/i-PrOH (2/1) and hydrogenated in the presence of Pd(OH)₂ (20 wt %) at 1 atm of H₂ gas pressure for 24 hrs at room temperature. The solution is filtered through a Celite pad. The filtrate is concentrated to give compound 45.

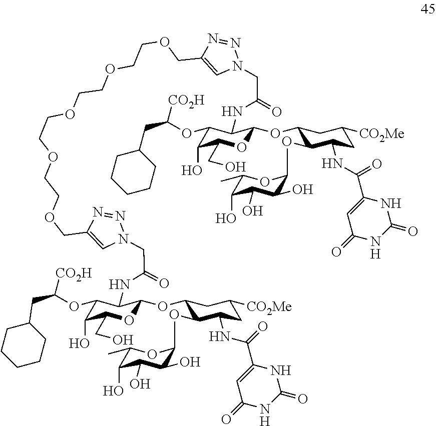

### Example 22

### Prophetic Synthesis of Multimeric Compound 46

Compound 46: Compound 45 is dissolved in ethylenediamine and stirred for 12 hrs at 70° C. The reaction mixture is concentrated under reduced pressure. The crude product is purified by C-18 column chromatography followed by lyophilization to give a compound 46.

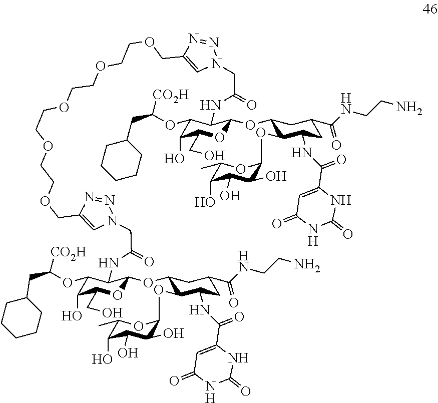

### Example 23

### Prophetic Synthesis of Multimeric Compound 47

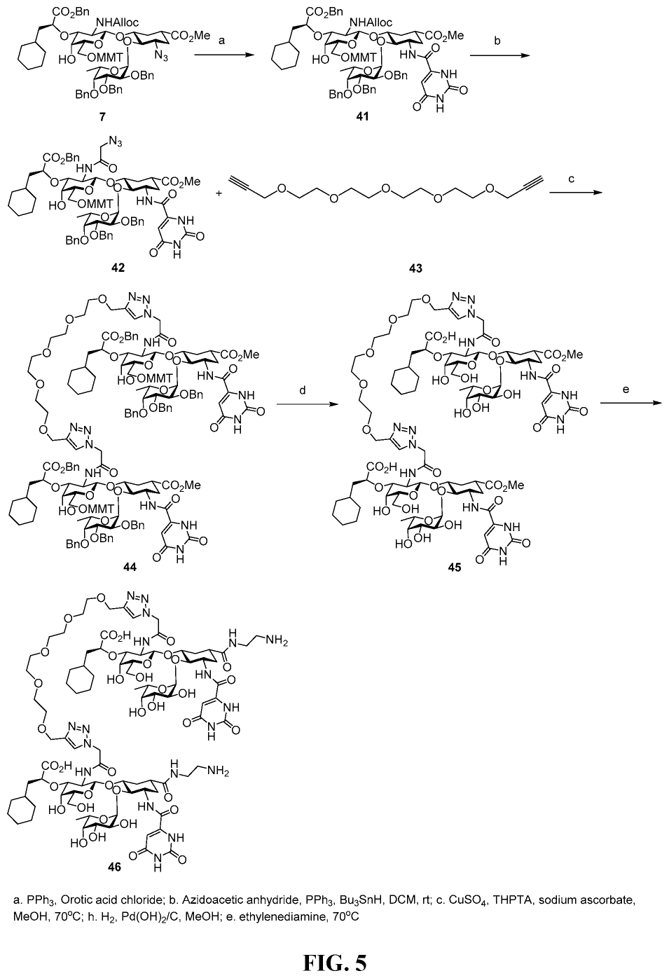

Compound 47: Compound 47 can be prepared in an analogous fashion to FIG. 5 using 3-azidopropanoic anhydride (Yang, C. et. al. JACS, (2013) 135(21), 7791-7794) in place of azidoacetic anhydride in step b.

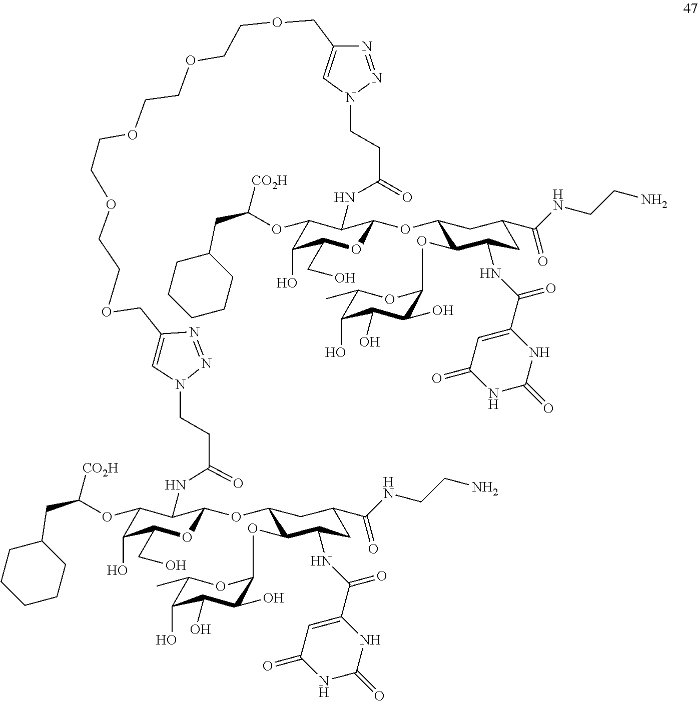

### Example 24

### Prophetic Synthesis of Multimeric Compound 48

Compound 48: Compound 48 can be prepared in an analogous fashion to FIG. 5 using 4-azidobutanoic anhydride (Yang, C. et. al. JACS, (2013) 135(21), 7791-7794) in place of azidoacetic anhydride in step b.

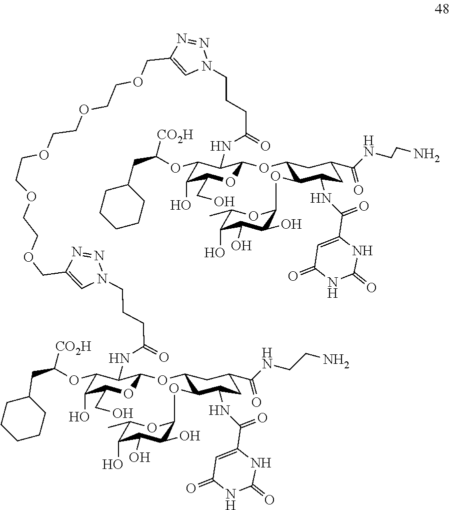

### Example 25

### Prophetic Synthesis of Multimeric Compound 49

Compound 49: Compound 49 can be prepared in an analogous fashion to FIG. 5 using 4-azidobutanoic anhydride (Yang, C. et. al. JACS, (2013) 135(21), 7791-7794) in place of azidoacetic anhydride in step b and using 1,2-bis(2-propynyloxy) ethane in place of compound 43 in step c.

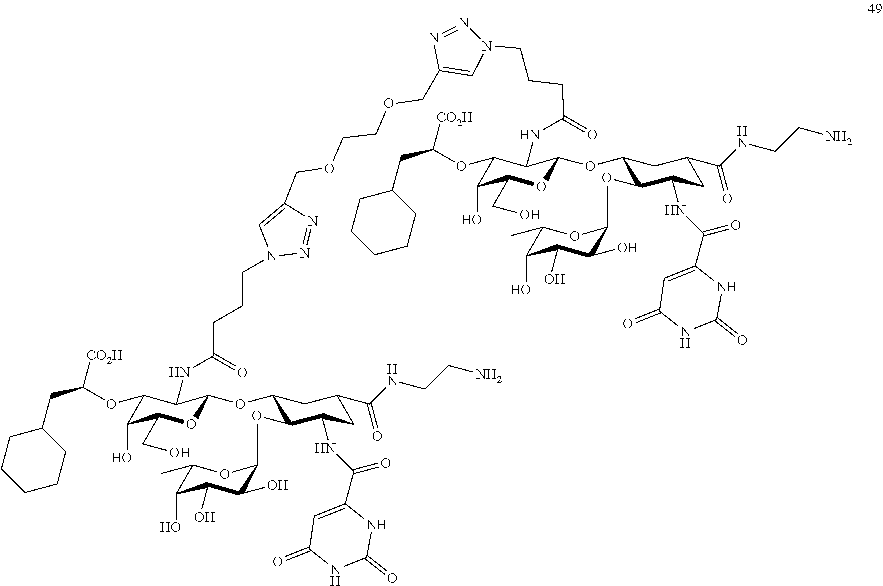

### Example 26

### Prophetic Synthesis of Multimeric Compound 50

Compound 50: Compound 50 can be prepared in an analogous fashion to FIG. 5 using 4,7,10,13,16,19,22,25,28,31-decaoxatetratriaconta-1, 33-diyne in place of compound 43 in step c.

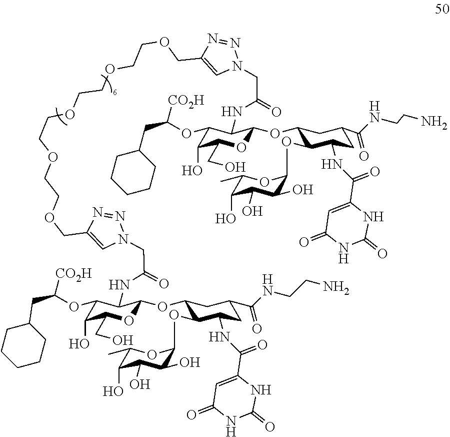

### Example 27

### Prophetic Synthesis of Multimeric Compound 51

Compound 51: Compound 51 can be prepared in an analogous fashion to FIG. 5 using 3,3′-[[2,2-bis[(2-propyn-1-yloxy)methyl]-1,3-propanediyl]bis(oxy)]bis-1-propyne in place of compound 43 in step c.

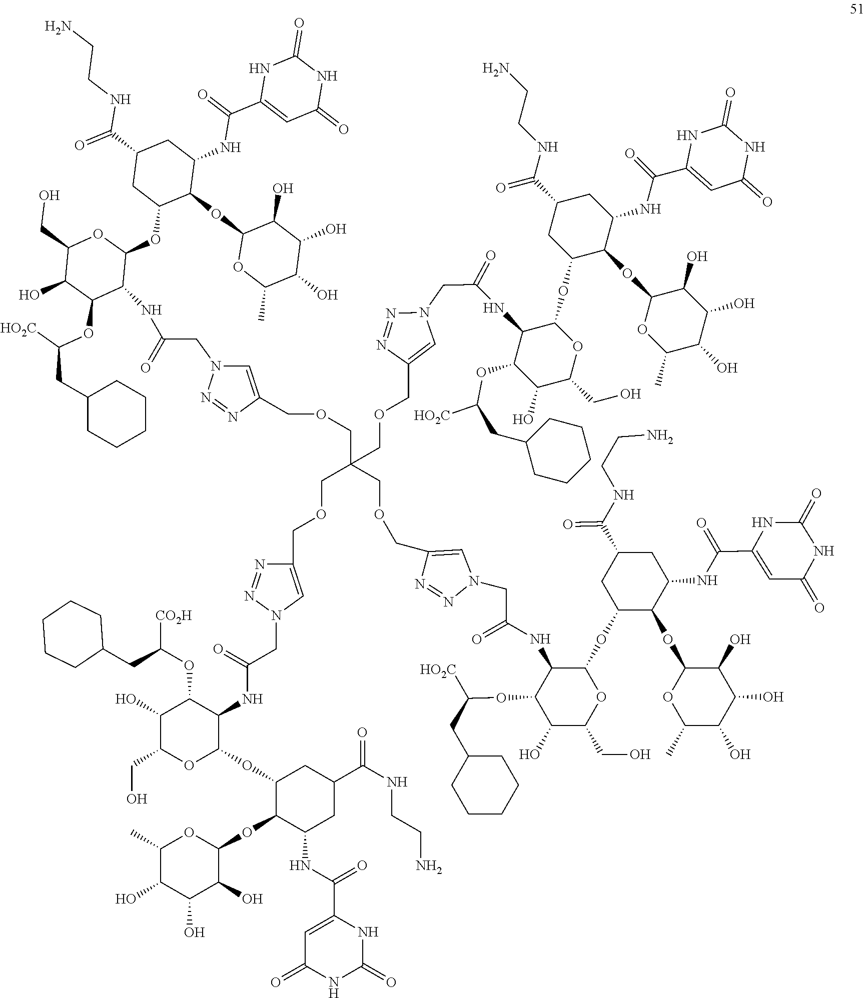

### Example 28

### Prophetic Synthesis of Multimeric Compound 52

Compound 52: Compound 52 can be prepared in an analogous fashion to FIG. 5 using 3,3′-[oxybis[[2,2-bis[(2-propyn-1-yloxy)methyl]-3,1-propanediyl]oxy]]bis-1-propyne in place of compound 43 in step c.

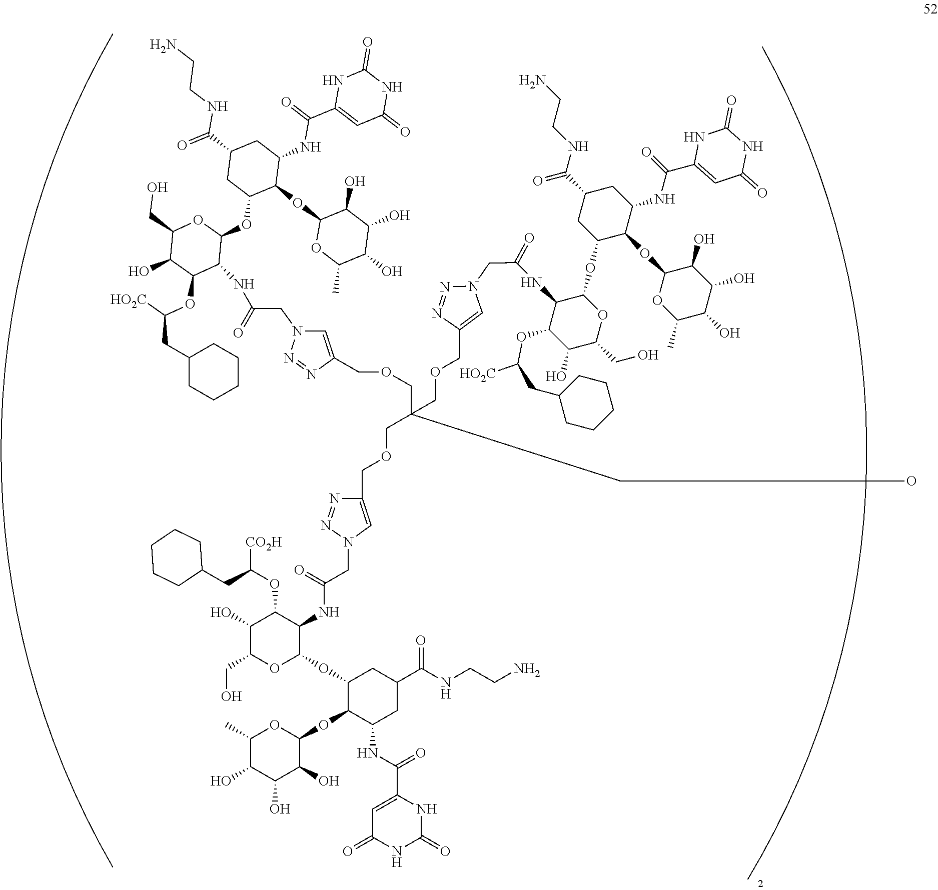

### Example 29

### Prophetic Synthesis of Multimeric Compound 53

Compound 53: Compound 53 can be prepared in an analogous fashion to FIG. 5 using butylenediamine in place of ethylenediamine in step e.

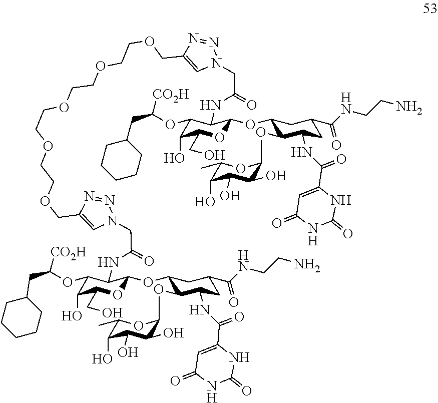

### Example 30

### Prophetic Synthesis of Multimeric Compound 54

Compound 54: Compound 54 can be prepared in an analogous fashion to FIG. 5 using 4-azidobutanoic anhydride (Yang, C. et. al. JACS, (2013) 135(21). 7791-7794) in place of azidoacetic anhydride in step b and using 1,2-bis(2-propynyloxy) ethane in place of compound 43 in step c and using 2-aminoethyl ether in step e.

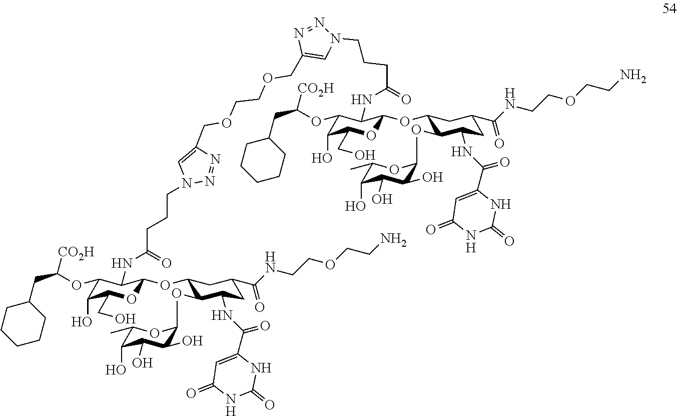

### Example 31

### Prophetic Synthesis of Multimeric Compound 55

Compound 55: Compound 54 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by flash chromatography to afford compound 55.

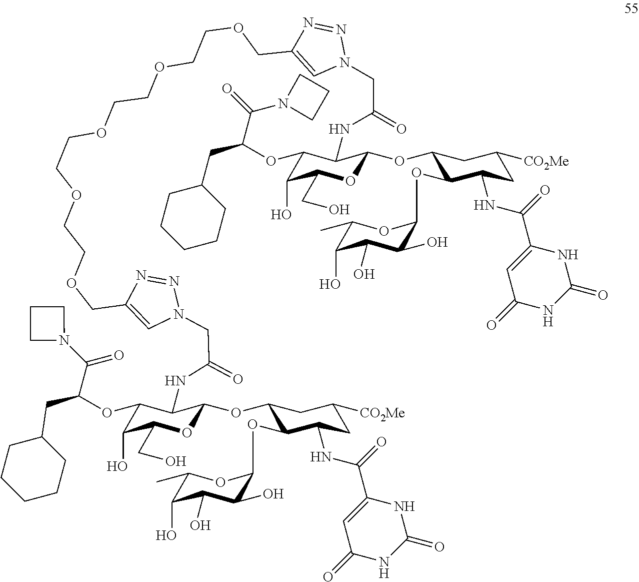

### Example 32

### Prophetic Synthesis of Multimeric Compound 56

Compound 56: Compound 55 is dissolved in ethylenediamine and stirred for 12 hrs at 70° C. The reaction mixture is concentrated under reduced pressure. The crude product is purified by C-18 column chromatography followed by lyophilization to give a compound 56.

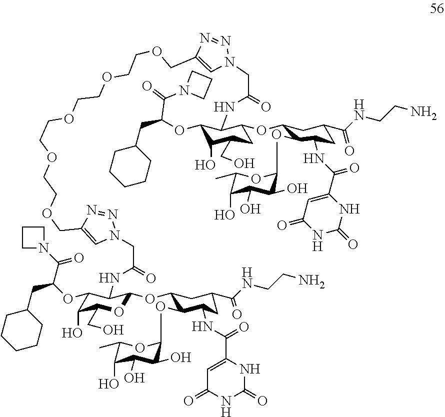

### Example 33 Prophetic Synthesis of Multimeric Compound 57

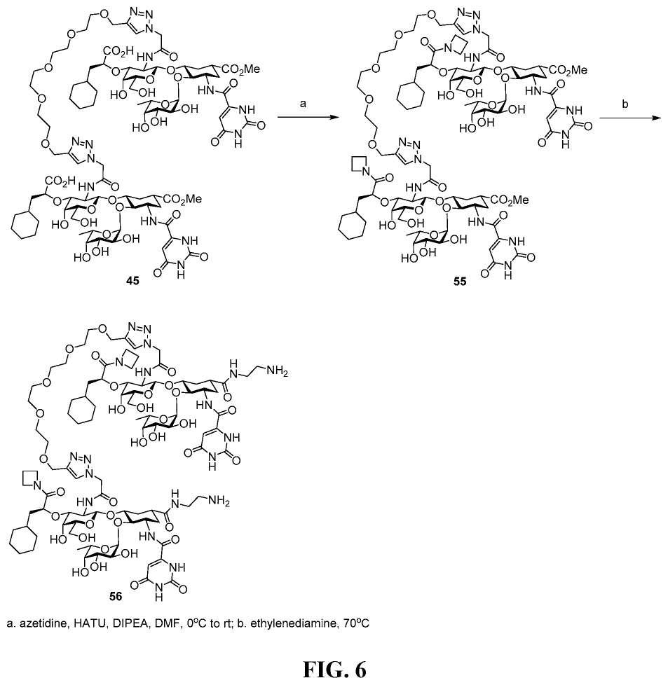

Compound 57: Compound 57 can be prepared in an analogous fashion to FIG. 6 using ethylamine in place of azetidine in step a.

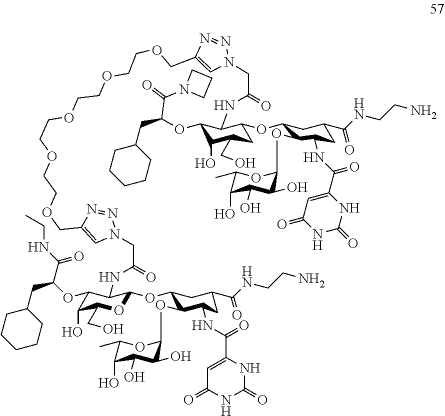

### Example 34

### Prophetic Synthesis of Multimeric Compound 58

Compound 58: Compound 58 can be prepared in an analogous fashion to FIG. 6 using dimethylamine in place of azetidine in step a.

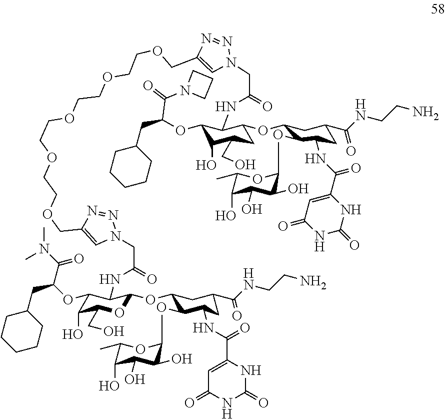

### Example 35

### Prophetic Synthesis of Multimeric Compound 59

Compound 59: Compound 59 can be prepared in an analogous fashion to FIG. 6 using 1,2-bis(2-aminoethoxy)ethane in place of ethylenediamine in step b.

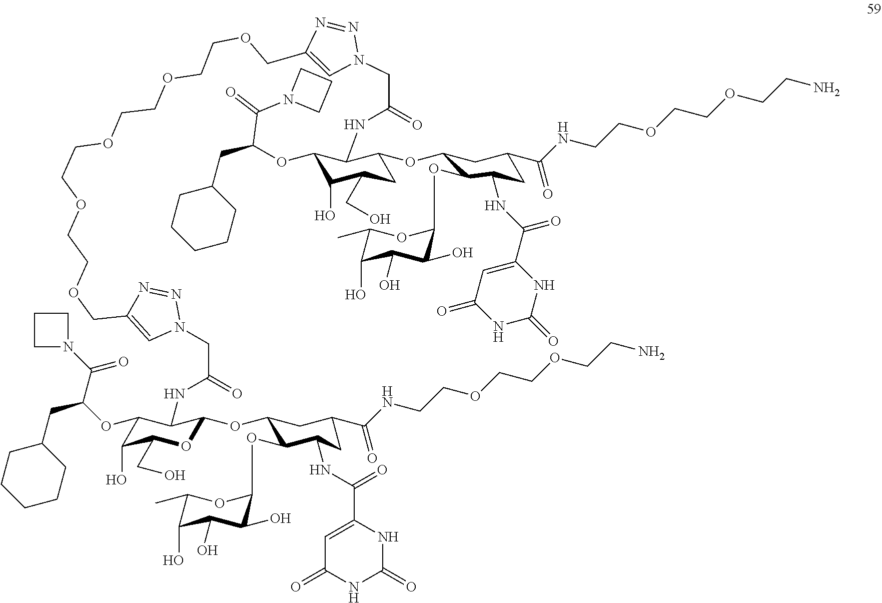

### Example 36

### Prophetic Synthesis of Multimeric Compound 66

Compound 60: To a stirred solution of compound 1 in DCM/MeOH (25/1) at room temperature is added orotic acid chloride (5 eq) and triphenylphosphine (5 eq). The reaction mixture is stirred 24 hours. The solvent is removed and the residue is separated by column chromatography to afford compound 60.

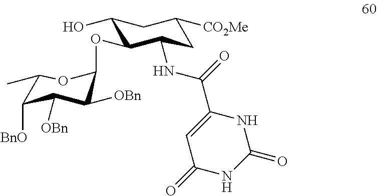

Compound 62: Compound 61 is dissolved in acetonitrile at room temperature. Benzaldehyde dimethylacetal (1.1 eq) is added followed by camphorsulfonic acid (0.2 eq). The reaction mixture is stirred until completion. Triethylamine is added. The solvent is removed and the residue separated by flash chromatography to afford compound 62.

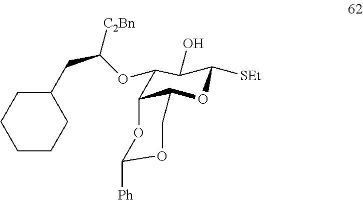

Compound 63: Compound 62 is dissolved in pyridine at room temperature. Dimethylaminopyridine (0.01 eq) is added followed by chloroacetyl chloride (2 eq). The reaction mixture is stirred until completion. The solvent is removed under educed pressure. The residue is dissolved in ethyl acetate, transferred to a separatory funnel and washed two times with 0.1N HCl and two times with water. The organic phase is dried over sodium sulfate, filtered, and concentrated. The residue is separated by column chromatograph to afford compound 63.

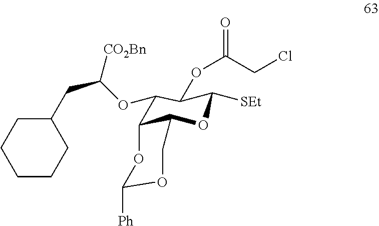

Compound 64: Activated powdered 4 Å molecular sieves are added to a solution of compound 60 and compound 63 (2 eq) in dry DCM under argon. The mixture is stirred for 2 hours at room temperature. Solid DMTST (1.5 eq) is added in 4 portions over 1.5 hours. The reaction mixture is stirred overnight at room temperature. The reaction mixture is filtered through Celite, transferred to a separatory funnel and washed two times with half saturated sodium bicarbonate and two times with water. The organic phase is dried over sodium sulfate, filtered and concentrated. The residue is separated by flash chromatography to afford compound 64.

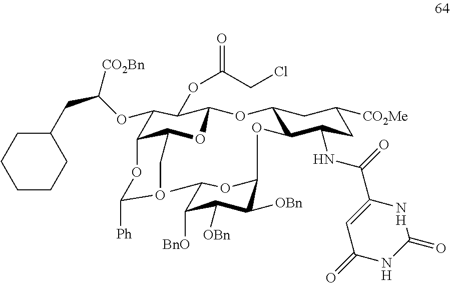

Compound 65: Compound 64 is dissolved in DMF. Sodium azide (1.5 eq) is added and the reaction mixture is stirred at 50° C. until completion. The reaction mixture is cooled to room temperature, diluted with ethyl acetate and transferred to a separatory funnel. The organic phase is washed 4 times with water then dried over sodium sulfate and concentrated. The residue is separated by column chromatography to afford compound 65.

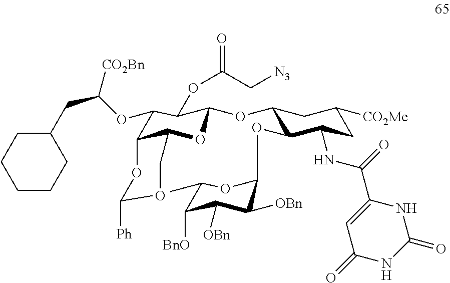

Compound 66: A solution of bispropagyl PEG-5 (compound 43) and compound 65 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred 12 hrs at 50° C. The solution is concentrated under reduced pressure. The crude product is purified by chromatography to give a compound 66.

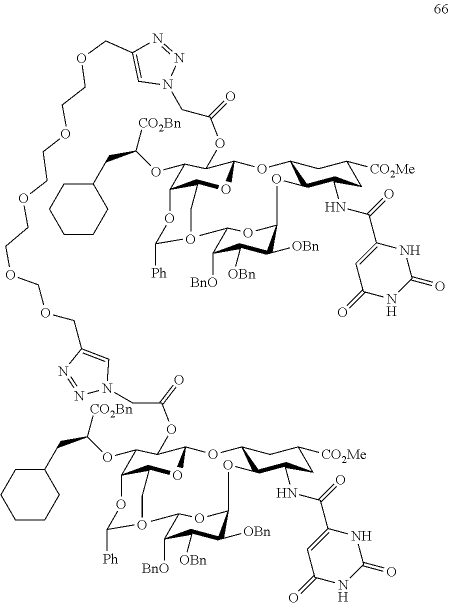

### Example 37

### Prophetic Synthesis of Multimeric Compound 67

Compound 67: To a solution of compound 66 in dioxane/water (4/1) is added Pd(OH)₂/C. The reaction mixture is stirred vigorously overnight under a hydrogen atmosphere. The reaction mixture is filtered through Celite and concentrated. The residue is purified by C-19 reverse phase column chromatography to afford compound 67.

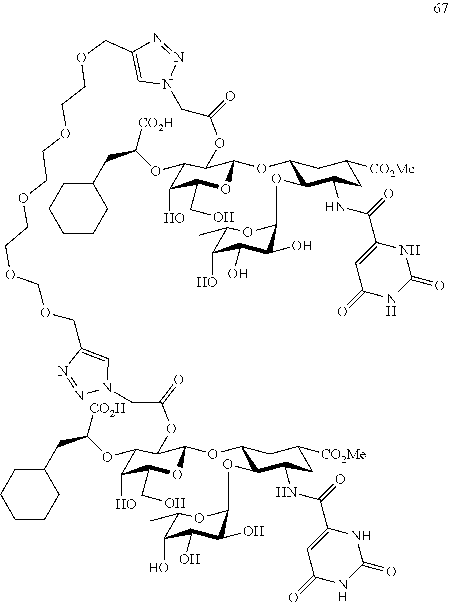

### Example 38

### Prophetic Synthesis of Multimeric Compound 68

Compound 68: Compound 67 is dissolved in ethylenediamine and stirred for 12 hrs at 70° C. The reaction mixture is concentrated under reduced pressure. The crude product is purified by C-18 column chromatography followed by lyophilization to afford compound 68.

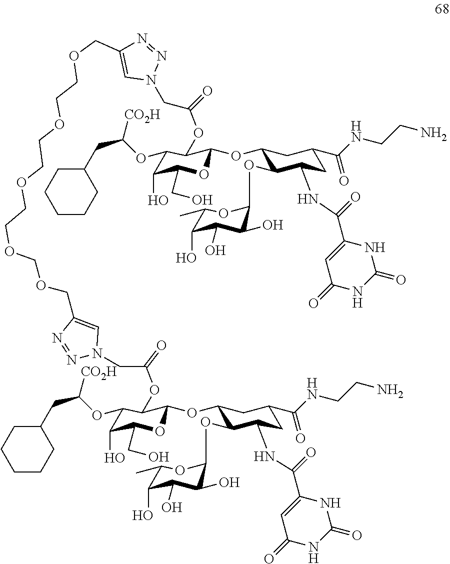

### Example 39

### Prophetic Synthesis of Multimeric Compound 69

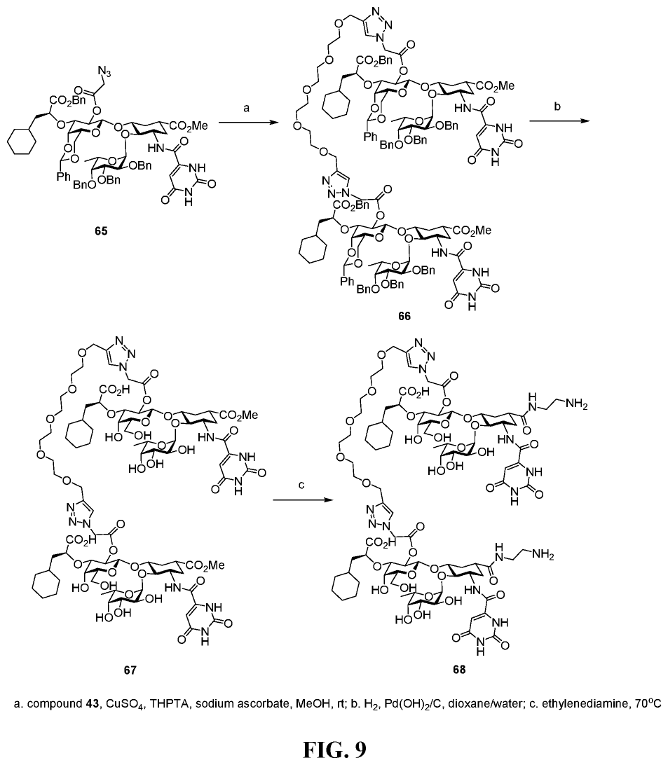

Compound 69: Compound 69 can be prepared in an analogous fashion to FIG. 9 by replacing compound 43 with PEG-8 bis propargyl ether in step a.

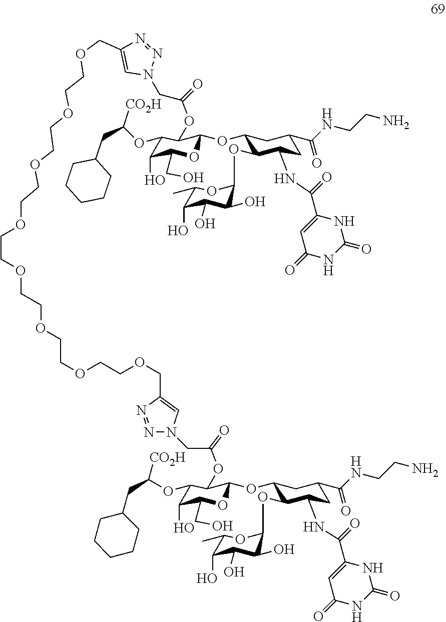

### Example 40

### Prophetic Synthesis of Multimeric Compound 70

Compound 70: Compound 70 can be prepared in an analogous fashion to FIG. 9 by replacing compound 43 with ethylene glycol bis propargyl ether in step a.

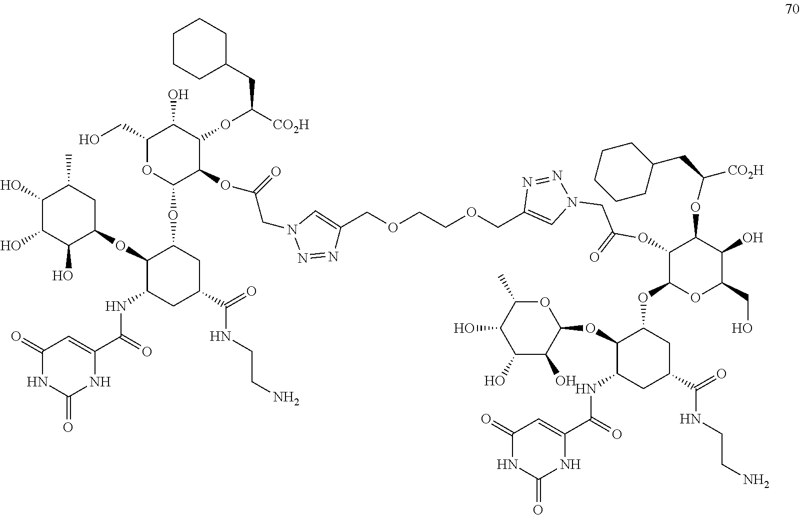

### Example 41

### Prophetic Synthesis of Multimeric Compound 71

Compound 71: Compound 71 can be prepared in an analogous fashion to FIG. 9 using; 3,3′-[[2,2-bis[(2-propyn-1-yloxy)methyl]-1,3-propanediyl]bis(oxy)]bis-1-propyne in place of compound 43 in step a.

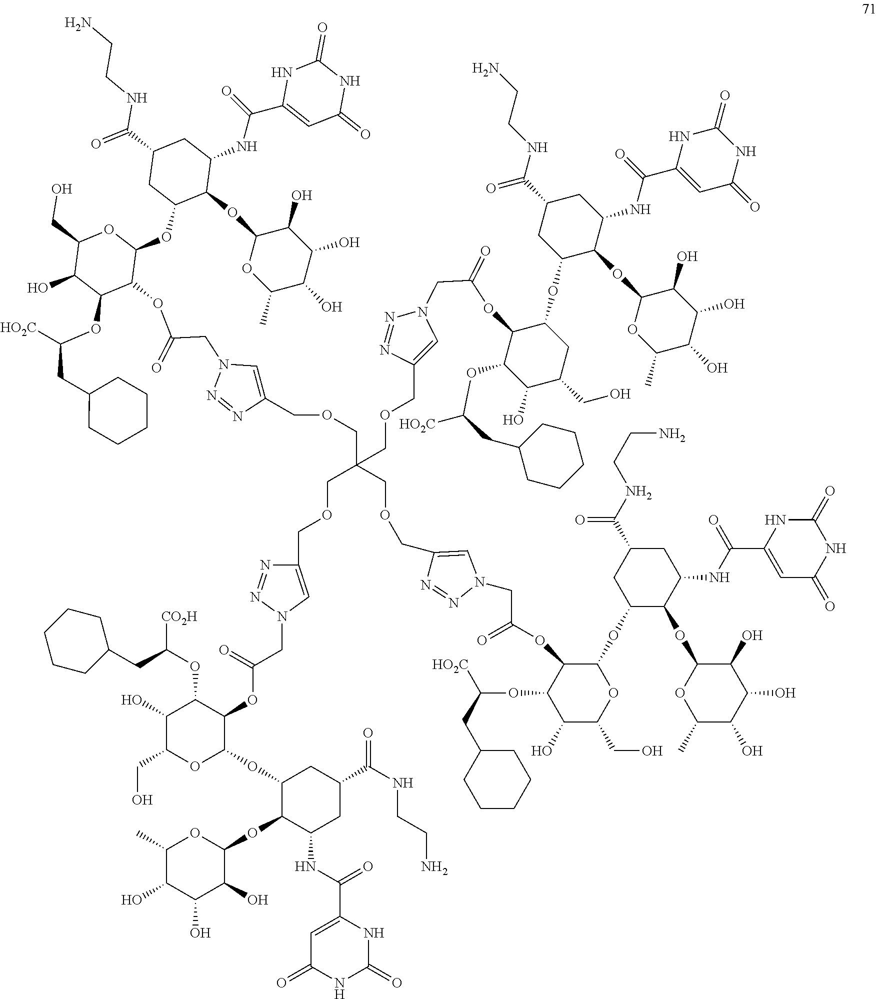

### Example 42

### Prophetic Synthesis of Multimeric Compound 72

Compound 72: Compound 67 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by flash chromatography to afford compound 72.

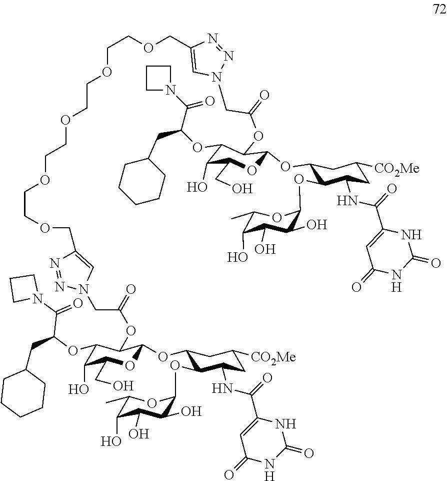

### Example 43

### Prophetic Synthesis of Multimeric Compound 73

Compound 73: Compound 72 is dissolved in ethylenediamine and stirred for 12 hrs at 70° C. The reaction mixture is concentrated under reduced pressure. The crude product is purified by C-18 column chromatography followed by lyophilization to afford compound 73.

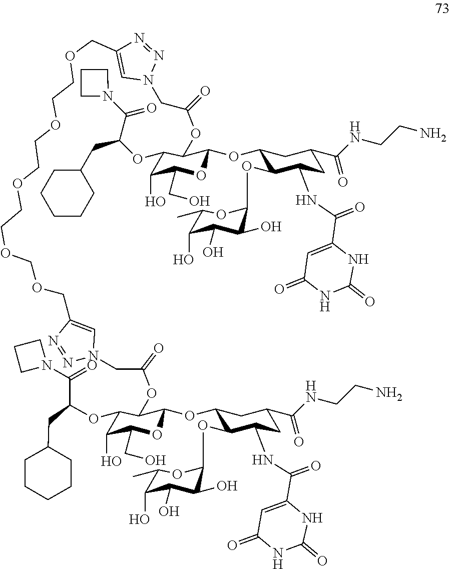

### Example 44

### Synthesis of Multimeric Compound 76

Compound 75: To a degassed solution of compound 74 (synthesis described in WO 2013/096926) (0.5 g, 0.36 mmole) in anhydrous DCM (10 mL) at 0° C. was added Pd(PPh₃)₄ (42 mg, 36.3 μmole, 0.1 eq), Bu₃SnH (110 μL, 0.4 μmole, 1.1 eq) and azidoacetic anhydride (0.14 g, 0.73 mmole, 2.0 eq). The resulting solution was stirred for 12 hrs under N₂ atmosphere while temperature was gradually increased to room temperature. After the reaction was completed, the solution was diluted with DCM (20 mL), washed with distilled water, dried over Na₂SO₄, then concentrated. The crude product was purified by combi-flash (EtOAc/Hex, Hex only—3/2, v/v) to give compound 75 (0.33 g, 67%). MS: Calculated (C₈₁H₉₅N₄O₁₆, 1376.6), ES-Positive (1400.4, M+Na)).

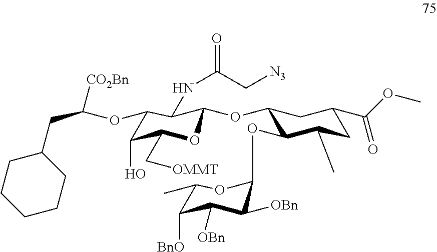

Compound 76: A solution of bispropargyl PEG-5 (compound 43, 27 mg, 0.1 mmole) and compound 75 (0.33 g, 0.24 mmole, 2.4 eq) in a mixed solution (MeOH/1,4 dioxane, 2/1, v/v, 12 mL) was degassed at room temperature. A solution of CuSO₄/THPTA in distilled water (0.04 M) (0.5 mL, 20 μmole, 0.2 eq) and sodium ascorbate (4.0 mg, 20 μmole, 0.2 eq) were added successively and the resulting solution was stirred 12 hrs at 70° C. The solution was cooled to room temperature and concentrated under reduced pressure. The crude product was purified by combi-flash (EtOAc/MeOH, EtOAc only—4/1, v/v) to give a compound 76 as a white foam (0.23 g, 70%).

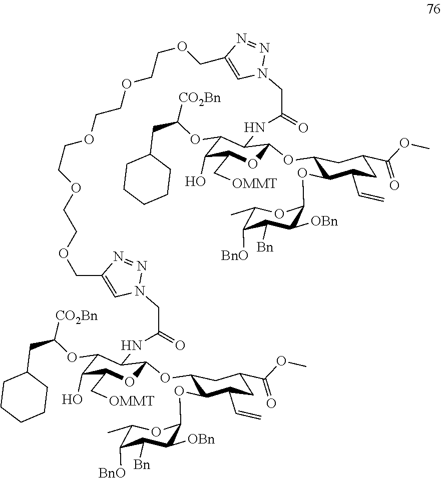

### Example 45

### Synthesis of Multimeric Compound 77

Compound 77: A solution of compound 76 (0.23 g, 0.76 μmole) in solution of MeOH/i-PrOH (2/1, v/v, 12 mL) was hydrogenated in the presence of Pd(OH)₂ (0.2 g) and 1 atm of H₂ gas pressure for 24 hrs at room temperature. The solution was filtered through a Celite pad and the cake was washed with MeOH. The combined filtrate was concentrated under reduced pressure. The crude product was washed with hexane and dried under high vacuum to give compound 77 as a white solid (0.14 g, quantitative). MS: Calculated (C₈₀H₁₃₀N₈O₃₅, 1762.8), ES− positive (1785.4, M+Na), ES− Negative (1761.5, M−1, 879.8).

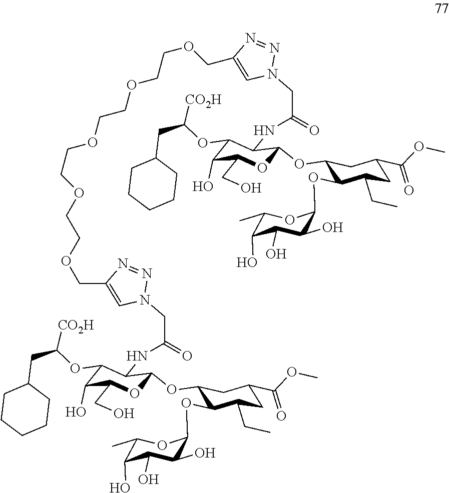

### Example 46

### Prophetic Synthesis of Multimeric Compound 78

Compound 78: Compound 77 (60 mg, 34.0 μmole) was dissolved in ethylenediamine (3 mL) and the homogeneous solution was stirred for 12 hrs at 70° C. The reaction mixture was concentrated under reduced pressure and the residue was dialyzed against distilled water with MWCO 500 dialysis tube. The crude product was further purified by C-18 column chromatography with water/MeOH (9/1-1/9, v/v) followed by lyophilization to give a compound 78 as a white solid (39 mg, 63%).

1H NMR (400 MHz, Deuterium Oxide) δ 8.00 (s, 2H), 5.26-5.14 (two d, J=16.0 Hz, 4H), 4.52 (d, J=4.0 Hz, 2H), 4.84 (dd, J=8.0 Hz, J=4.0 Hz, 2H), 4.66 (s, 4H), 4.54 (broad d, J=12 Hz, 2H), 3.97 (broad t, 2H), 3.91-3.78 (m, 6H), 3.77-3.58 (m, 28H), 3.57-3.46 (m, 4H), 3.42 (t, J=8.0 Hz, 6H), 3.24 (t, J=12.0 Hz, 2H), 3.02 (t, J=6.0 Hz, 4H), 2.67 (s, 2H), 2.32 (broad t, J=12 Hz, 2H), 2.22-2.06 (m, 2H), 1.96-1.74 (m, 4H), 1.73-1.39 (m, 18H), 1.38-1.21 (m, 6H), 1.20-0.99 (m, J=8.0 Hz, 14H), 0.98-0.73 (m, J=8.0 Hz, 10H).

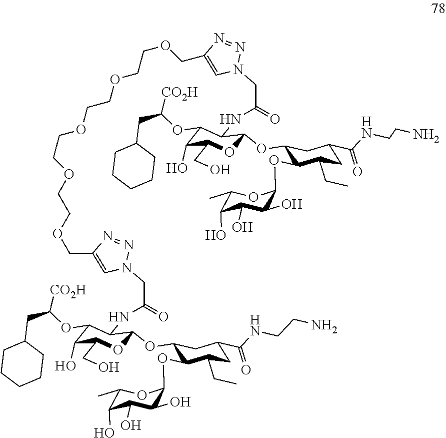

### Example 47

### Prophetic Synthesis of Multimeric Compound 79

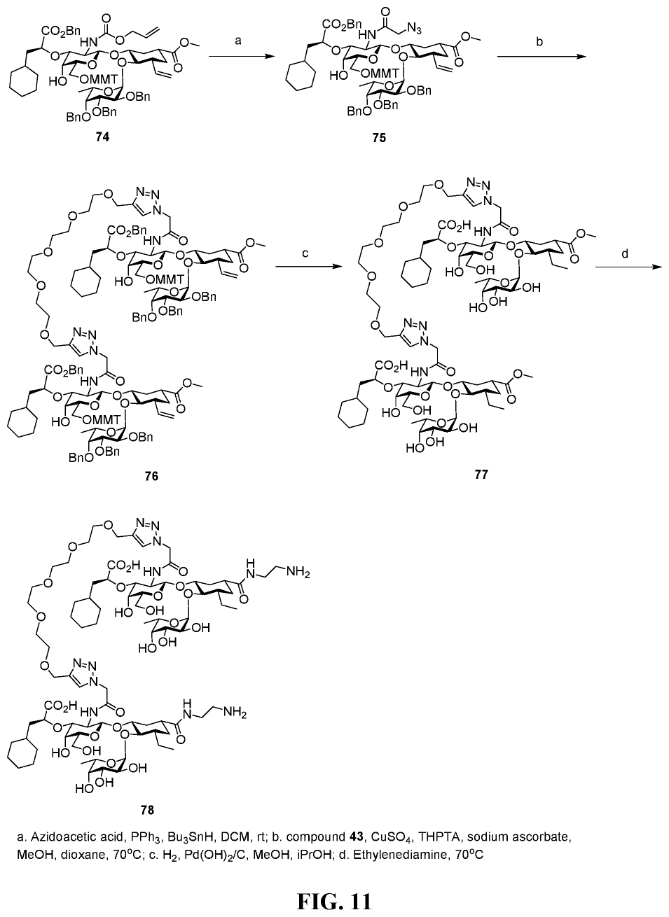

Compound 79: Compound 79 can be prepared in an analogous fashion to FIG. 11 using 3-azidopropanoic anhydride (Yang, C. et. al. JACS, (2013) 135(21), 7791-7794) in place of azidoacetic anhydride in step a.

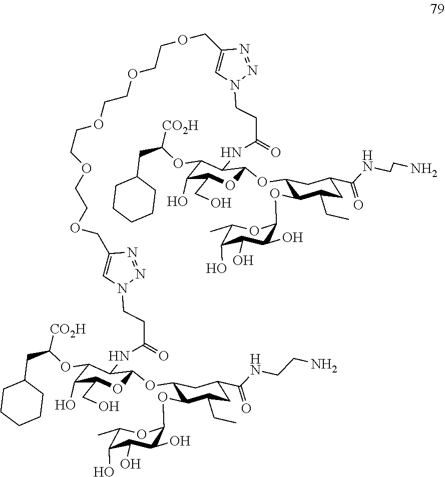

### Example 48

### Prophetic Synthesis of Multimeric Compound 80

Compound 80: Compound 80 can be prepared in an analogous fashion to FIG. 11 using 4-azidobutanoic anhydride (Yang, C. et. al. JACS, (2013) 135(21), 7791-7794) in place of azidoacetic anhydride in step a.

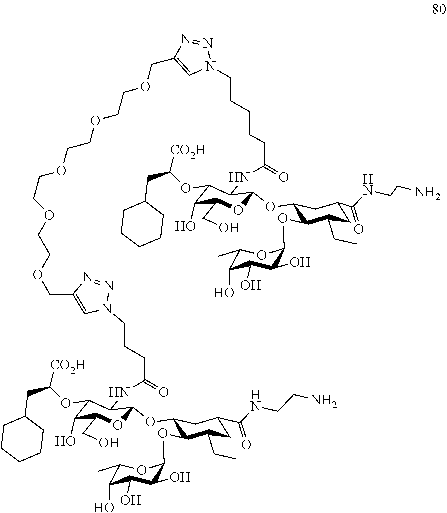

### Example 49

### Prophetic Synthesis of Multimeric Compound 81

Compound 81: Compound 81 can be prepared in an analogous fashion to FIG. 11 using 4-azidobutanoic anhydride (Yang, C. et. al. JACS, (2013) 135(21), 7791-7794) in place of azidoacetic anhydride in step a and using 1,2-bi(2-propynyloxy) ethane in place of compound 43 in step b.

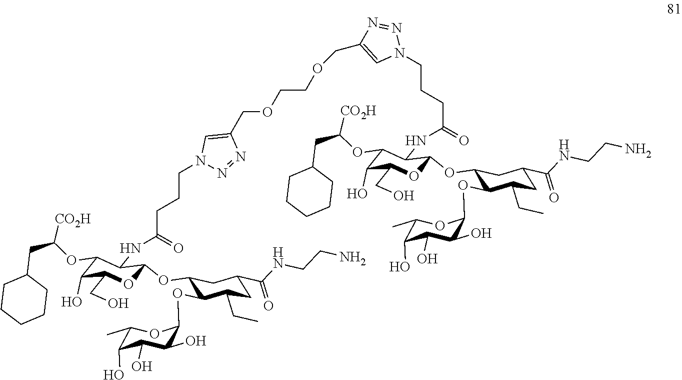

### Example 50

### Prophetic Synthesis of Multimeric Compound 82

Compound 82: Compound 82 can be prepared in an analogous fashion to FIG. 11 using 4,7,10,13,16,19,22,25,28,31-decaoxatetratriaconta-1, 33-diyne in place of compound 43 in step b.

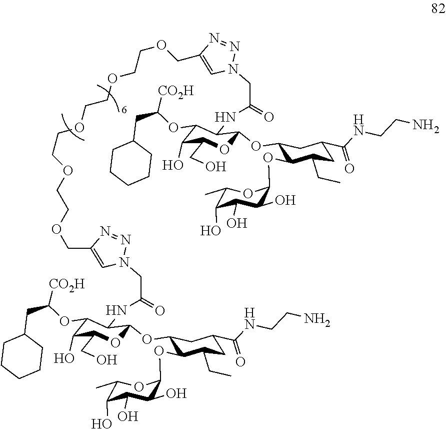

### Example 51

### Prophetic Synthesis of Multimeric Compound 83

Compound 83: Compound 83 can be prepared in an analogous fashion to FIG. 11 using 2-aminoethylether in place of ethylenediamine in step d.

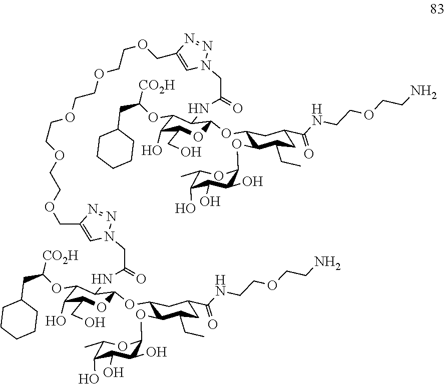

### Example 52

### Prophetic Synthesis of Multimeric Compound 84

Compound 84: Compound 84 can be prepared in an analogous fashion to FIG. 11 using 1,2-bi(2-propynyloxy) ethane in place of compound 43 in step b.

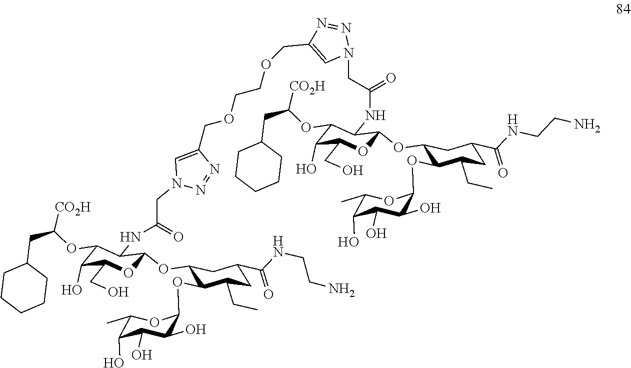

### Example 53

### Prophetic Synthesis of Multimeric Compound 85

Compound 85: Compound 85 can be prepared in an analogous fashion to FIG. 11 using PEG-8 dipropargyl ether in place of compound 43 in step b and 1,5-diaminopentane in place of ethylenediamine in step d.

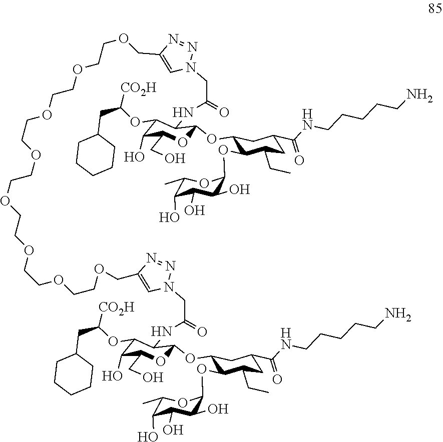

### Example 54

### Prophetic Synthesis of Multimeric Compound 86

Compound 86: Compound 77 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by flash chromatography to afford compound 86.

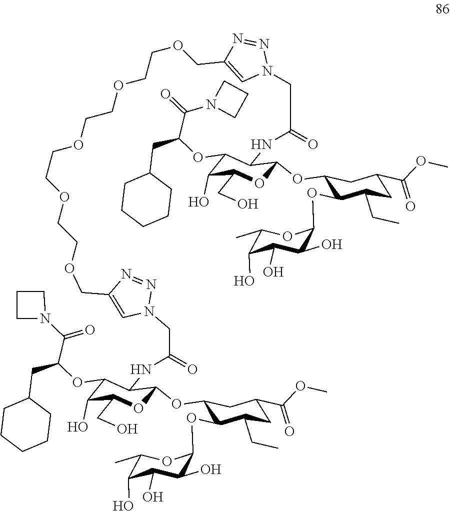

### Example 55

### Prophetic Synthesis of Multimeric Compound 87

Compound 87: Compound 86 is dissolved in ethylenediamine stirred for 12 hrs at 70° C. The reaction mixture was concentrated under reduced pressure. The residue was purified by C-18 column chromatography followed by lyophilization to give a compound 87.

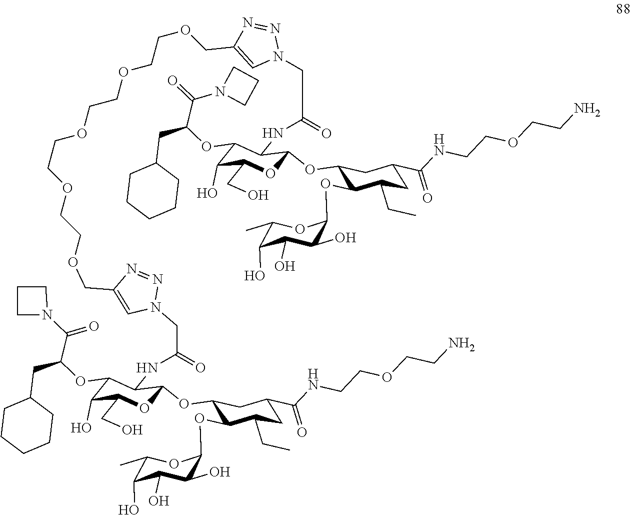

### Example 56

### Prophetic Synthesis of Multimeric Compound 88

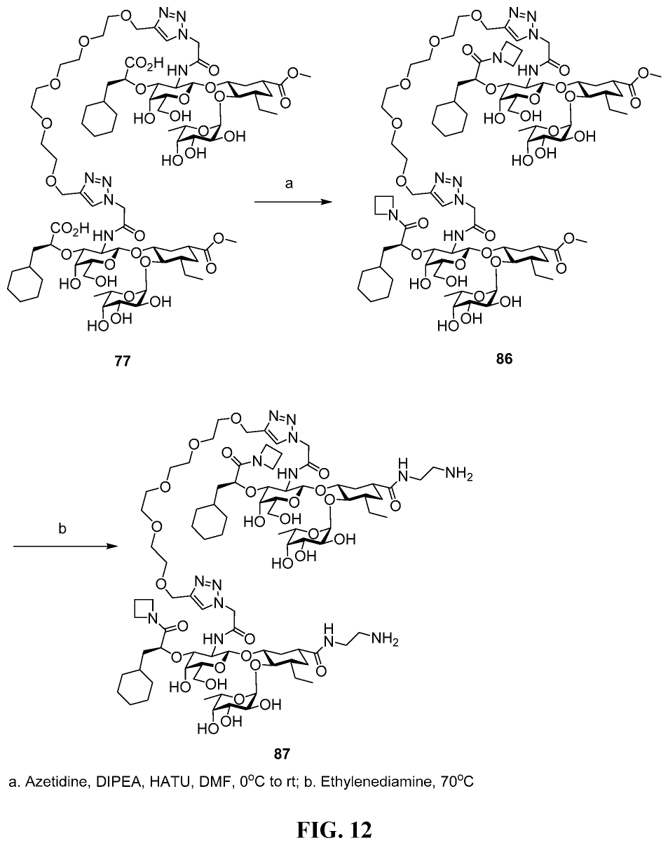

Compound 88: Compound 88 can be prepared in an analogous fashion to FIG. 12 using 2-aminoethylether in place of ethylenediamine in step b.

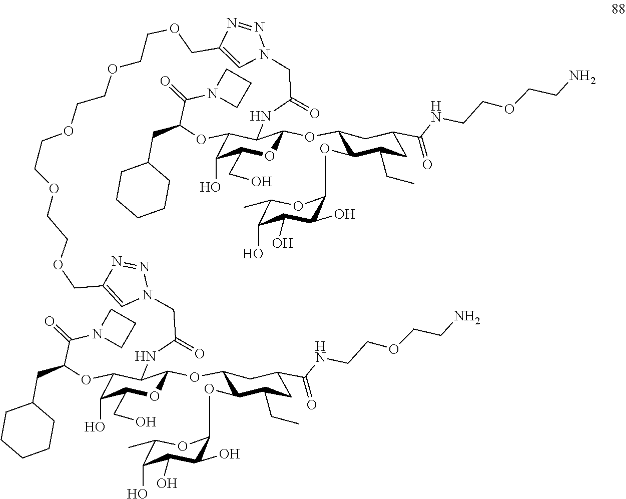

### Example 57

### Prophetic Synthesis of Multimeric Compound 89

Compound 89: Compound 89 can be prepared in an analogous fashion to FIG. 12 using dimethylamine in place of azetidine in step a and 2-aminoethylether in place of ethylenediamine in step b.

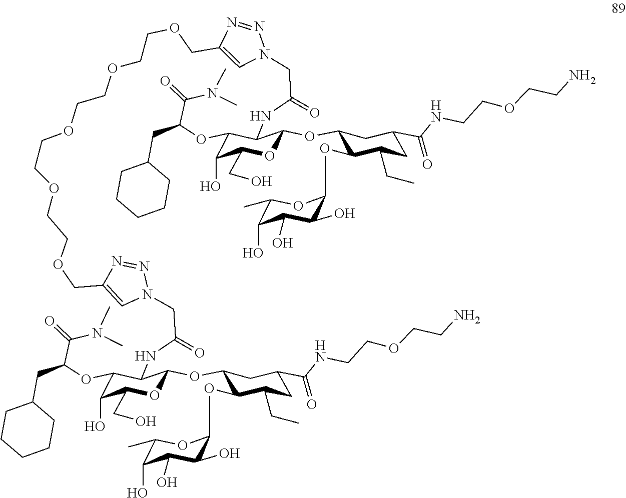

### Example 58

### Prophetic Synthesis of Multimeric Compound 90

Compound 90: Compound 90 can be prepared in an analogous fashion to FIG. 12 using piperidine in place of azetidine in step a.

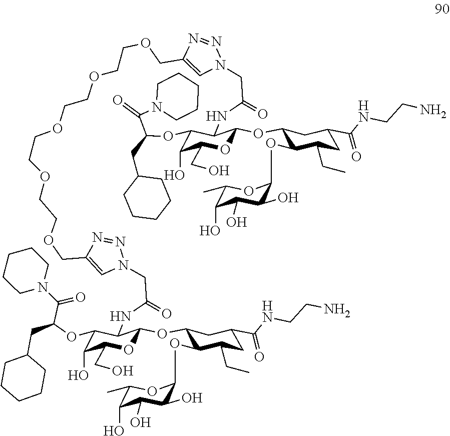

### Example 59

### Prophetic Synthesis of Multimeric Compound 91

Compound 91: Compound 91 can be prepared in an analogous fashion to FIGS. 11 and 12 using in PEG-9 bis-propargyl ether in place of compound 43 in step b of Scheme 11.

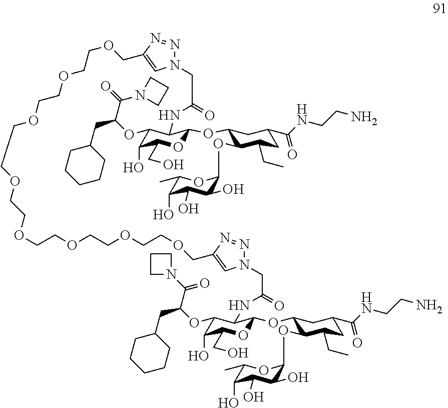

### Example 60

### Prophetic Synthesis of Multimeric Compound 92

Compound 92: Compound 92 can be prepared in an analogous fashion to FIGS. 11 and 12 using 1,2-bi(2-propynyloxy) ethane in place of compound 43 in step b in Scheme 11.

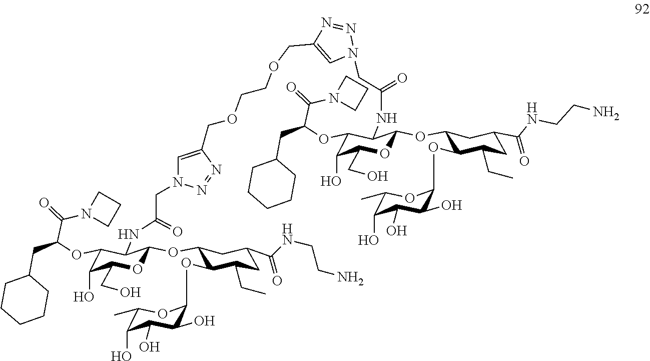

### Example 61

### Prophetic Synthesis of Multimeric Compound 93

Compound 93: Compound 93 can be prepared in an analogous fashion to FIGS. 11 and 12 using 1,2-bi(2-propynyloxy) ethane in place of compound 43 in step b in Scheme 11 and using 2-aminoethyl ether in place of ethylenediamine in step b of Scheme 12.

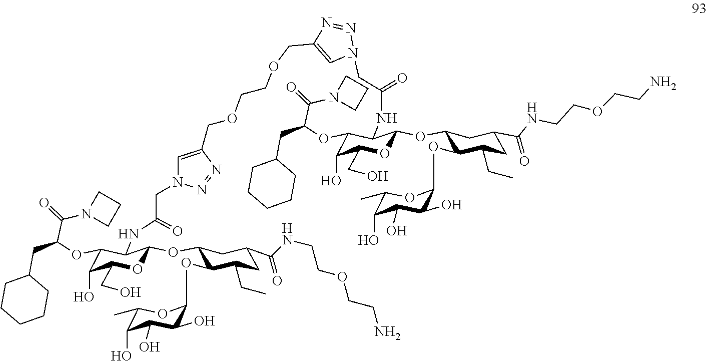

### Example 62

### Synthesis of Multimeric Compound 95

Compound 95: Compound 22 and compound 94 (5 eq)(preparation described in WO/2016089872) is co-evaporated 3 times from methanol and stored under vacuum for 1 hour. The mixture is dissolved in methanol under an argon atmosphere and stirred for 1 hour at room temperature. Sodium triacetoxyborohydride (15 eq) is added and the reaction mixture is stirred overnight at room temperature. The solvent is removed and the residue is separated by C-18 reverse phase chromatography.

The purified material is dissolved in methanol at room temperature. The pH is adjusted to 12 with 1N NaOH. The reaction mixture is stirred at room temperature until completion. The pH is adjusted to 9. The solvent is removed under vacuum and the residue is separated by C-18 reverse phase chromatography to afford compound 95.

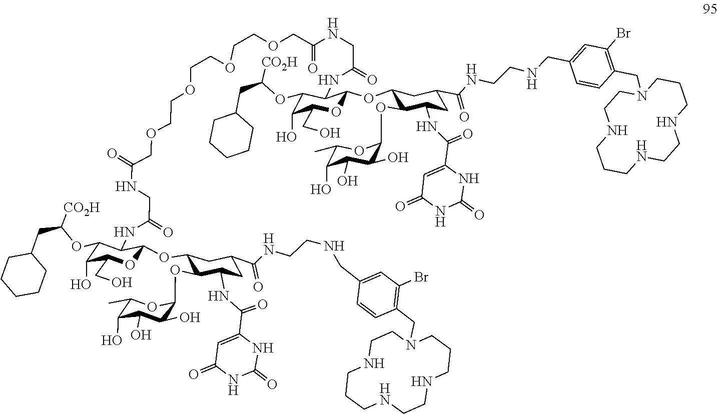

### Example 63

### Prophetic Synthesis of Multimeric Compound 96

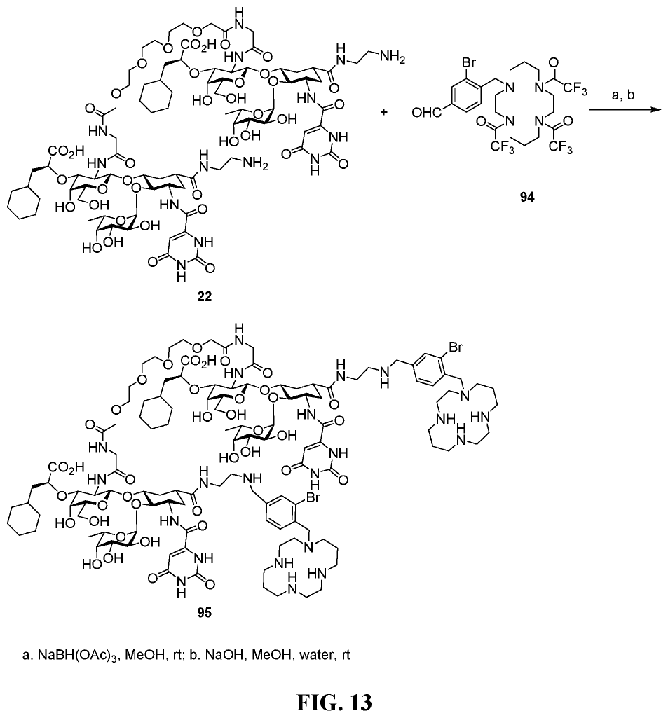

Compound 96: Compound 96 can be prepared in an analogous fashion to FIG. 13 by replacing compound 22 with compound 23 in step a

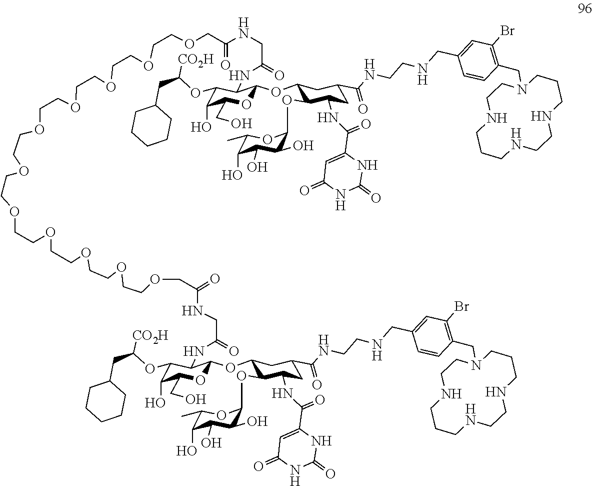

### Example 64

### Prophetic Synthesis of Multimeric Compound 97

Compound 97: Compound 97 can be prepared in an analogous fashion to FIG. 13 by replacing compound 22 with compound 24 in step a.

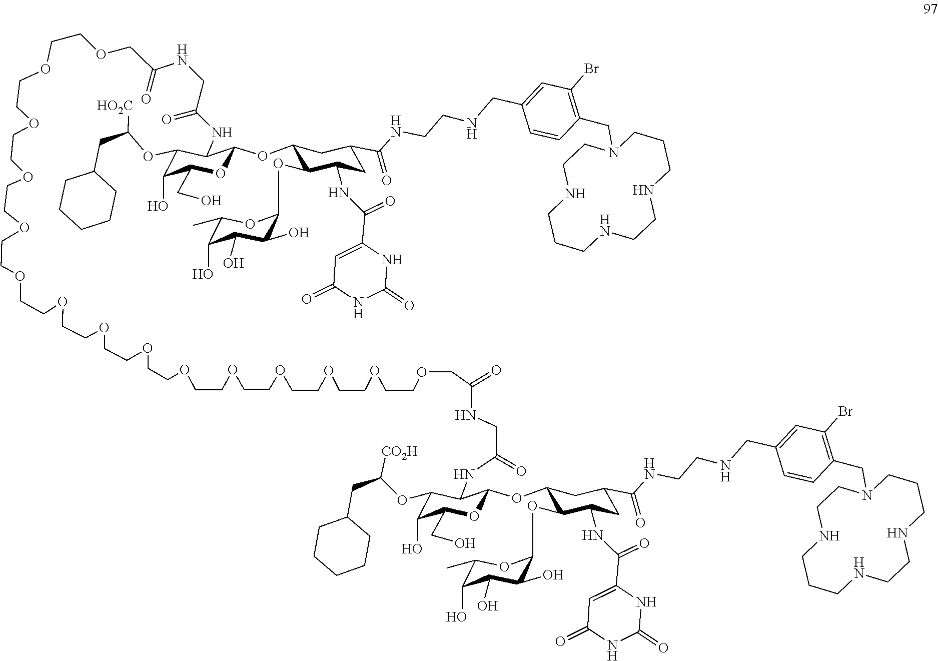

### Example 65

### Prophetic Synthesis of Multimeric Compound 98

Compound 98: Compound 98 can be prepared in an analogous fashion to FIG. 13 by replacing compound 22 with compound 25 in step a.

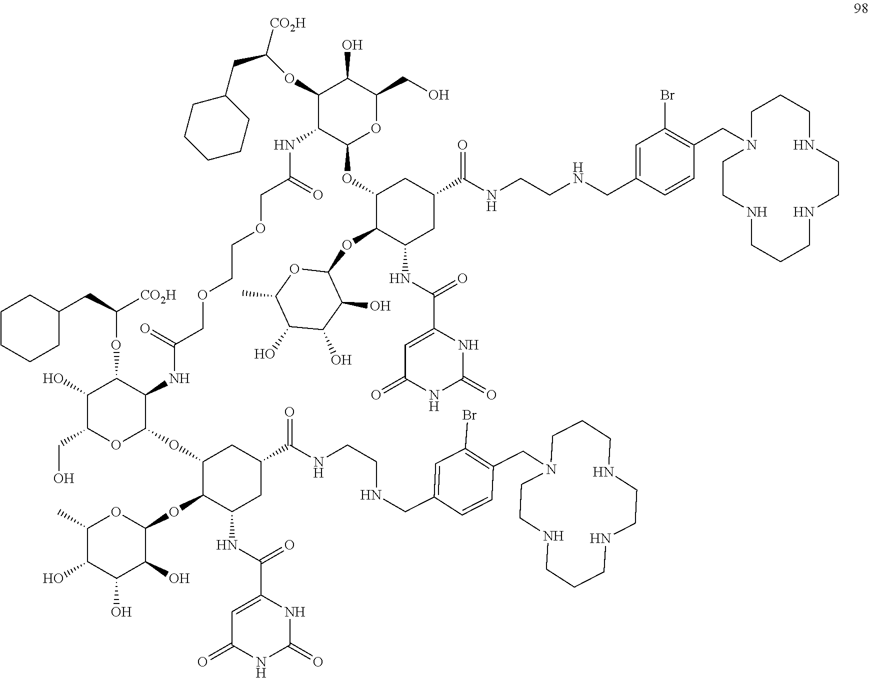 [SMILES: C[C@@H]1O[C@@H](O[C@@H]2[C@@H](NC(=O)c3cc(=O)[nH]c(=O)[nH]3)C[C@@H](C(=O)NCCNCc3ccc(CN4CCCNCCNCCCNCC4)c(Br)c3)C[C@H]2O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3C[C@H](C(=O)NCCNCc4ccc(CN5CCCNCCNCCCNCC5)c(Br)c4)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H](O)[C@H](O)[C@@H]1O]

### Example 66

### Prophetic Synthesis of Multimeric Comound 99

Compound 99: Compound 99 can be prepared in an analogous fashion to FIG. 13 by replacing compound 22 with compound 26 in step a

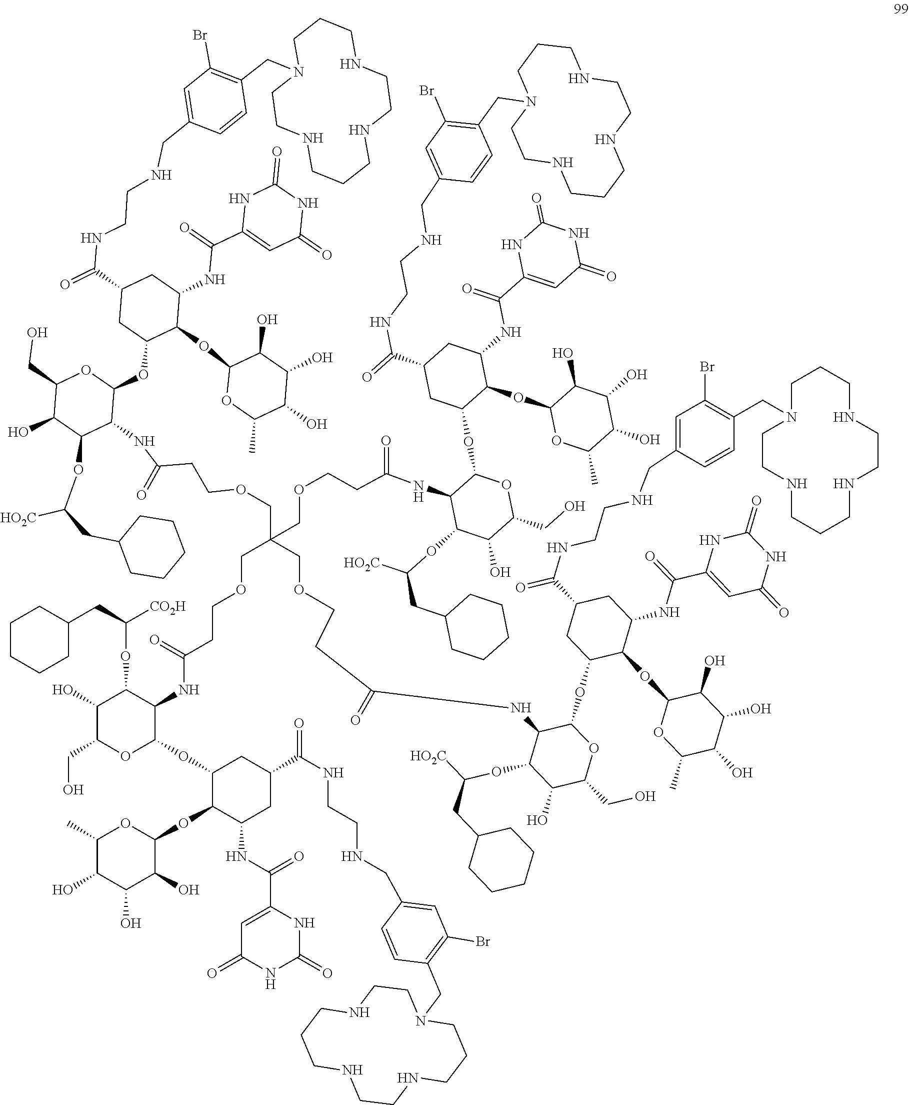

### Example 67

### Prophetic Synthesis of Multimeric Compound 100

Compound 100: Compound 100 can be prepared in an analogous fashion to FIG. 13 by replacing compound 22 with compound 27 in step a.

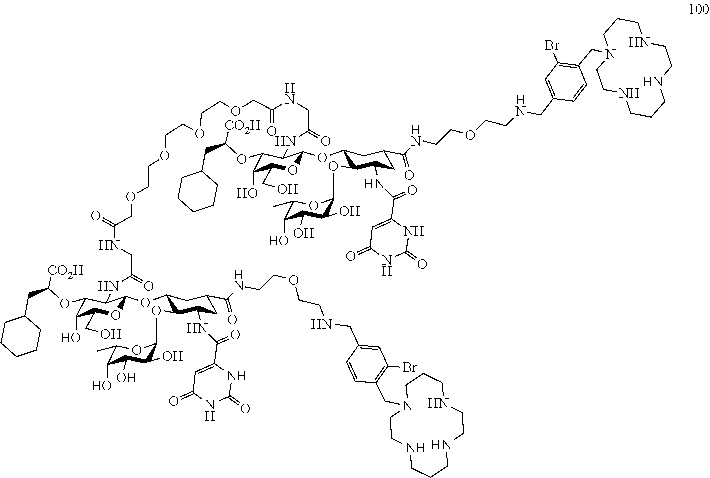

### Example 68

### Prophetic Synthesis of Multimeric Compound 101

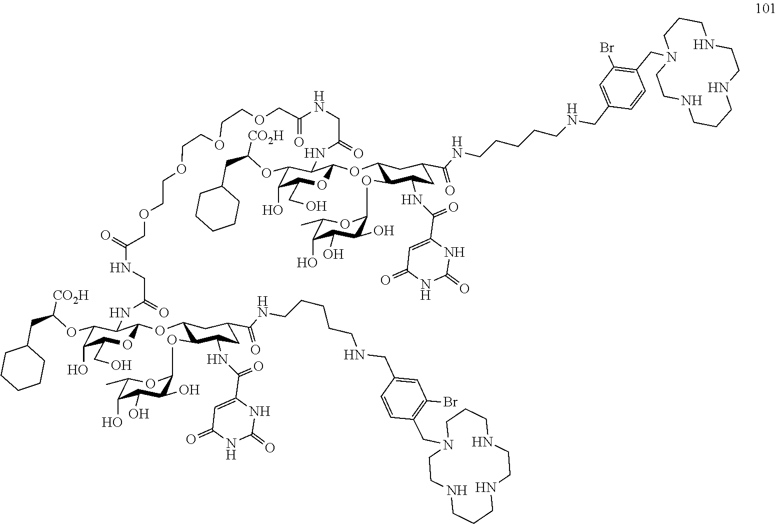

Compound 102: Compound 102 can be prepared in an analogous fashion to FIG. 13 by replacing compound 22 with compound 29 in step a.

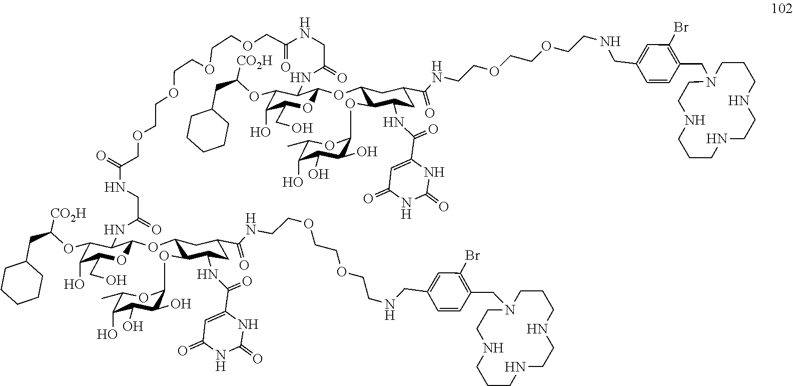

### Example 70

### Prophetic Synthesis of Multimeric Compound 103

Compound 103: Compound 103 can be prepared in an analogous fashion to FIG. 13 by replacing compound 22 with compound 30 in step a.

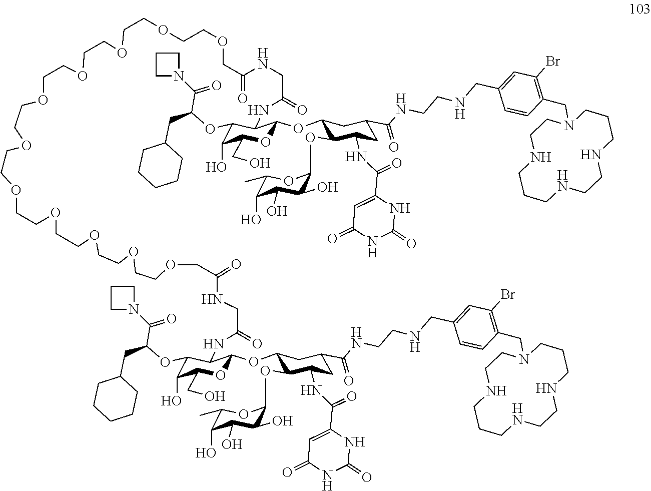

### Example 71

### Prophetic Synthesis of Multimeric Compound 104

Compound 104: Compound 104 can be prepared in an analogous fashion to FIG. 13 by replacing compound 22 with compound 31 in step a.

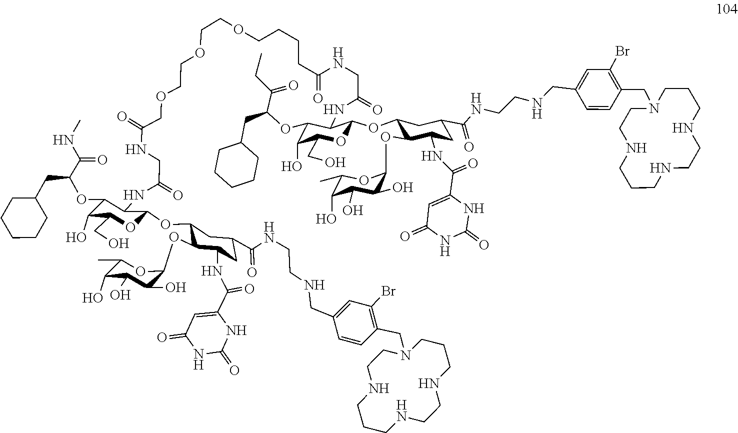

### Example 72

### Prophetic Synthesis of Multimeric Compound 105

Compound 105: Compound 105 can be prepared in an analogous fashion to FIG. 13 by replacing compound 22 with compound 32 in step a.

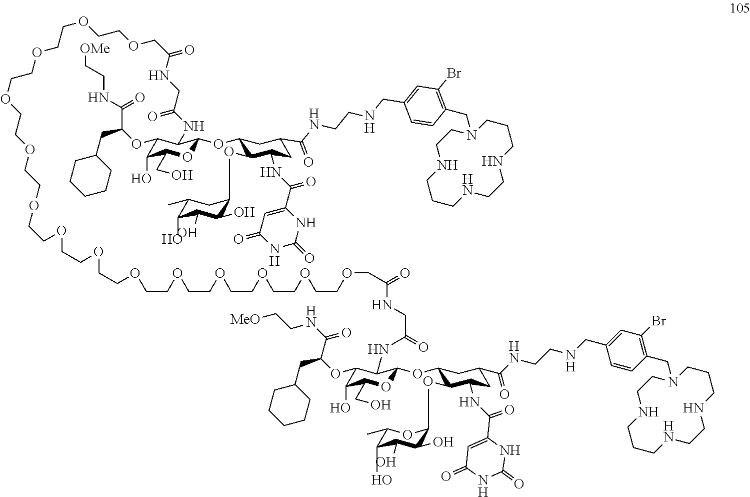

### Example 73

### Prophetic Synthesis of Multimeric Compound 106

Compound 106: Compound 106 can be prepared in an analogous fashion to FIG. 13 by replacing compound 22 with compound 33 in step a.

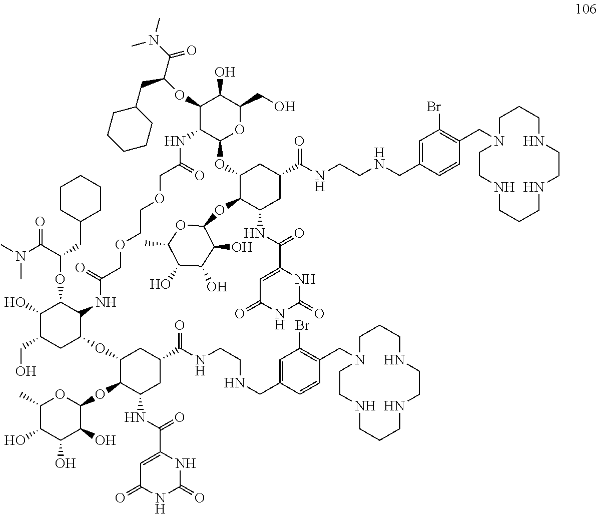

### Example 74

### Prophetic Synthesis of Multimeric Compound 107

Compound 107: Compound 107 can be prepared in an analogous fashion to FIG. 13 by replacing compound 22 with compound 34 in step a.

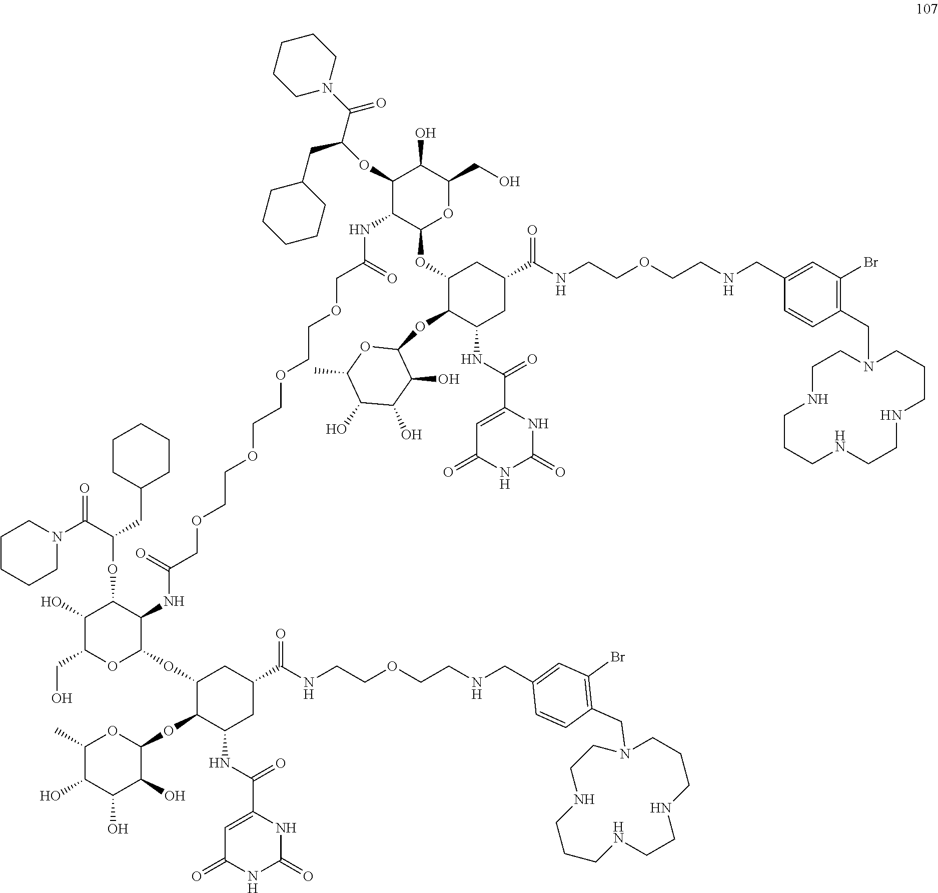 [SMILES: C[C@@H]1O[C@@H](O[C@@H]2[C@@H](NC(=O)c3cc(=O)[nH]c(=O)[nH]3)C[C@@H](C(=O)NCCOCCNCc3ccc(CN4CCCNCCNCCCNCC4)c(Br)c3)C[C@H]2O[C@@H]2O[C@H](CO)[C@H](O)[C@H](O[C@@H](CC3CCCCC3)C(=O)N3CCCCC3)[C@H]2NC(=O)COCCOCCOCCOCC(=O)N[C@H]2[C@H](O[C@@H]3C[C@H](C(=O)NCCOCCNCc4ccc(CN5CCCNCCNCCCNCC5)c(Br)c4)C[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCCCC2)[C@@H](O)[C@H](O)[C@@H]1O]

### Example 75

### Prophetic Synthesis of Multimeric Compound 108

Compound 108: Compound 108 can be prepared in an analogous fashion to FIG. 13 by replacing compound 22 with compound 37 in step a.

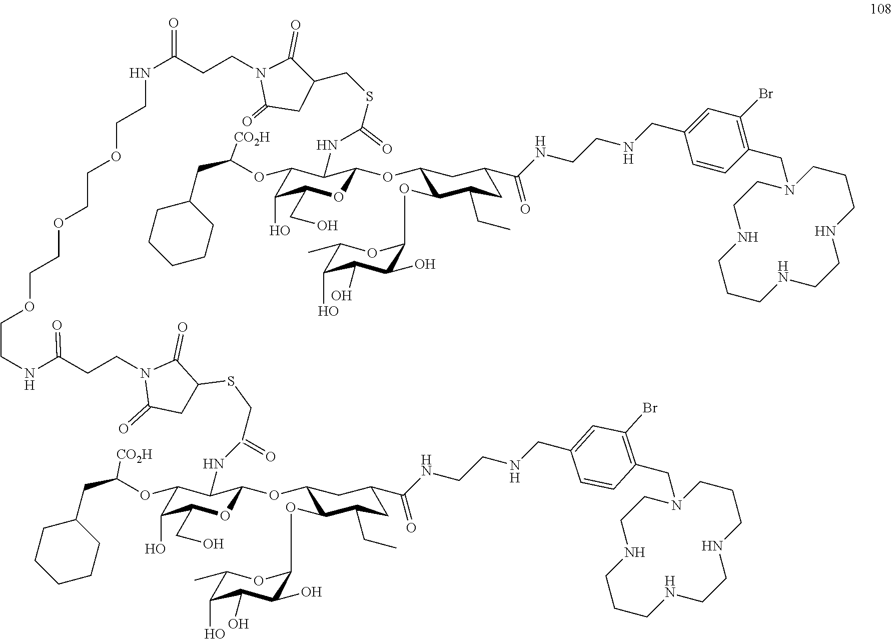

### Example 76

### Prophetic Synthesis of Multimeric Compound 109

Compound 109: Compound 109 can be prepared in an analogous fashion to FIG. 13 by replacing compound 22 with compound 38 in step a.

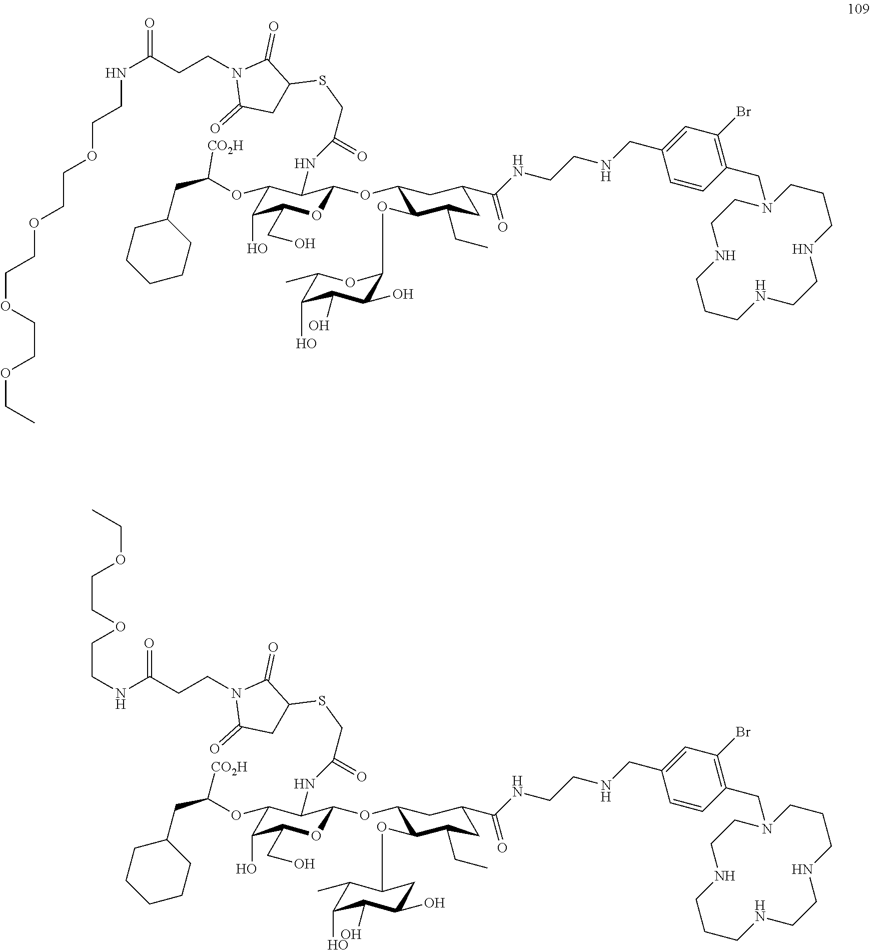

### Example 77

### Prophetic Synthesis of Multimeric Compound 110

Compound 110: Compound 110 can be prepared in an analogous fashion to FIG. 13 by replacing compound 22 with compound 39 in step a.

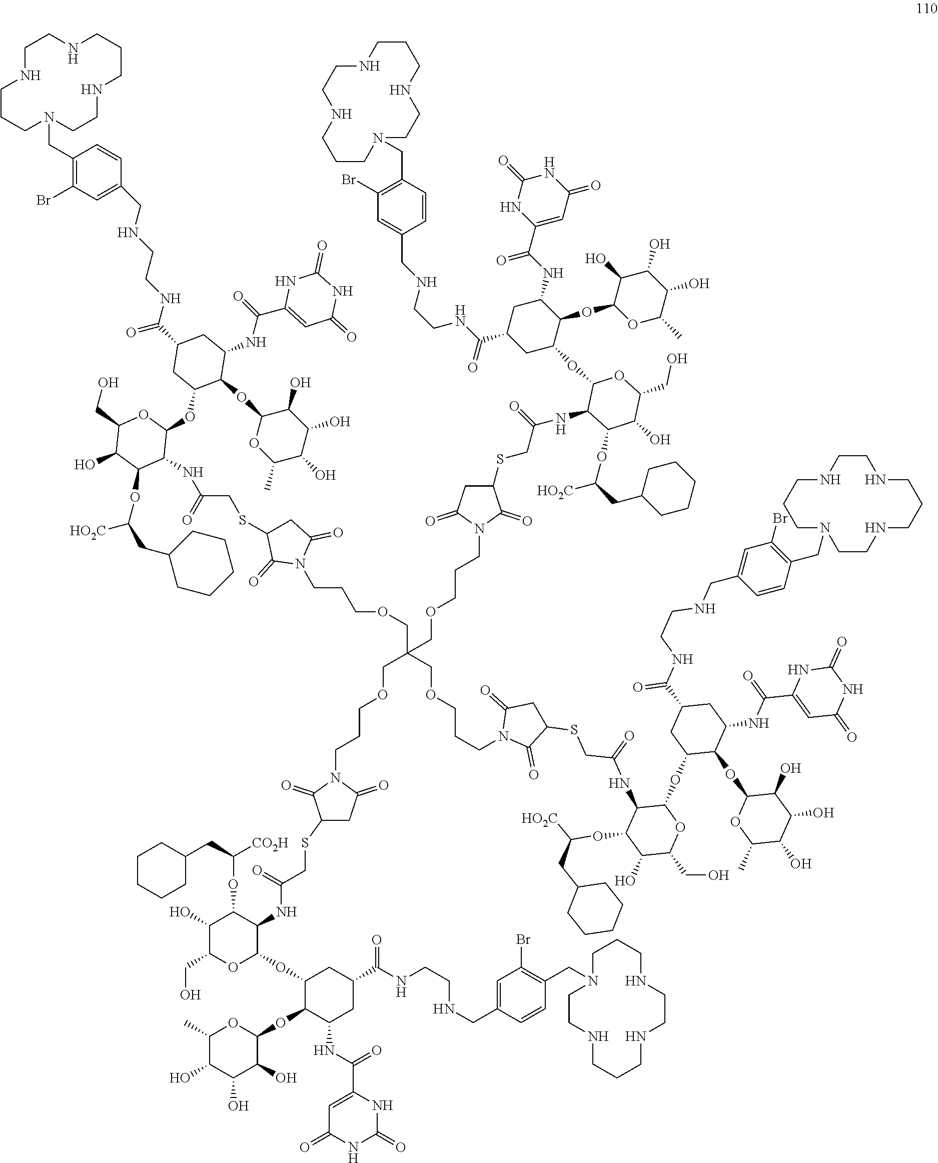

### Prophetic Synthesis of Multimeric Compound 111

Compound 111: Compound 111 can be prepared in an analogous fashion to FIG. 13 by replacing compound 22 with compound 40 in step a.

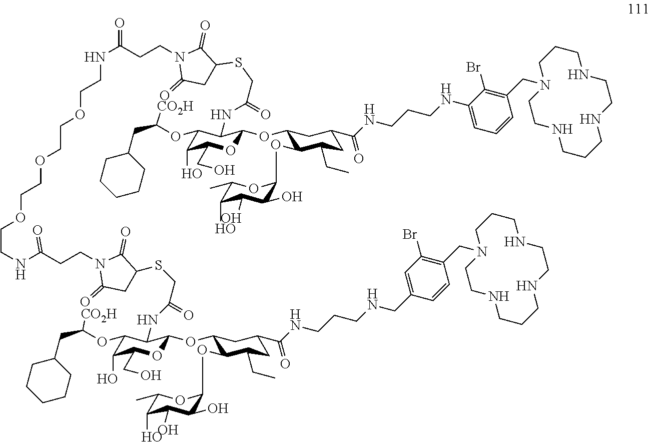

### Example 78

### Prophetic Synthesis of Multimeric Compound 112

Compound 112: Compound 112 can be prepared in an analogous fashion to FIG. 13 by replacing compound 22 with compound 46 in step a.

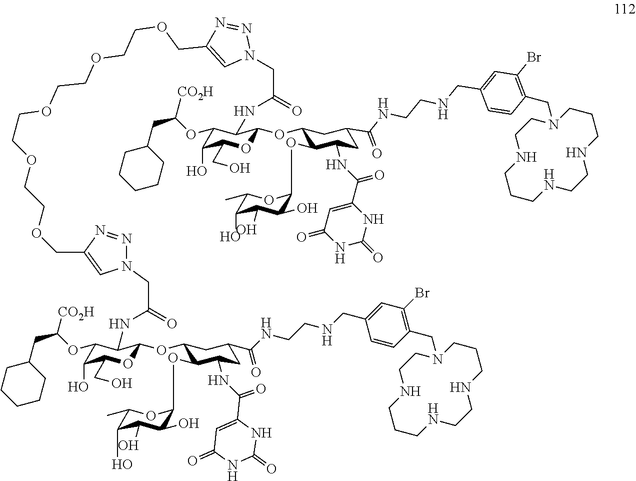

### Example 79

### Prophetic Synthesis of Multimeric Compound 113

Compound 113: Compound 113 can be prepared in an analogous fashion to FIG. 13 by replacing compound 22 with compound 47 in step a.

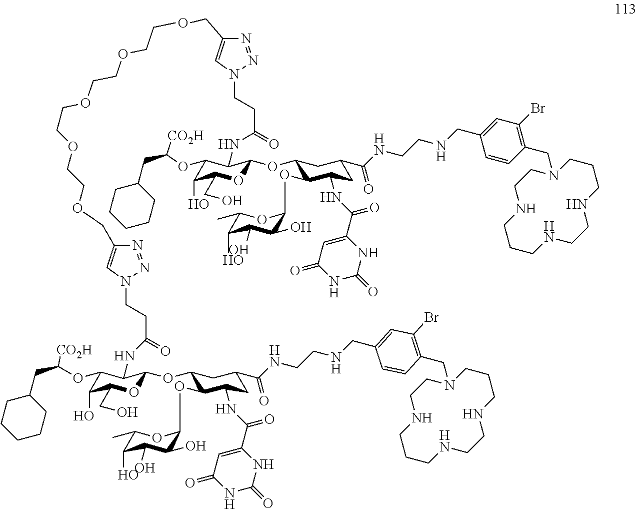

### Example 80

### Prophetic Synthesis of Multimeric Compound 114

Compound 114: Compound 114 can be prepared in an analogous fashion to FIG. 13 by replacing compound 22 with compound 48 in step a.

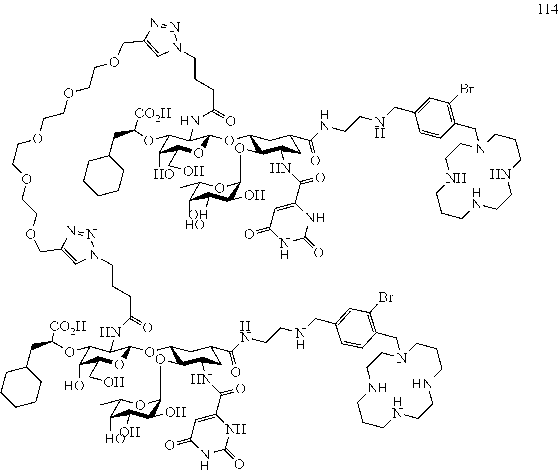

### Example 81

### Prophetic Synthesis of Multimeric Compound 115

Compound 115: Compound 115 can be prepared in an analogous fashion to FIG. 13 by replacing compound 22 with compound 49 in step a.

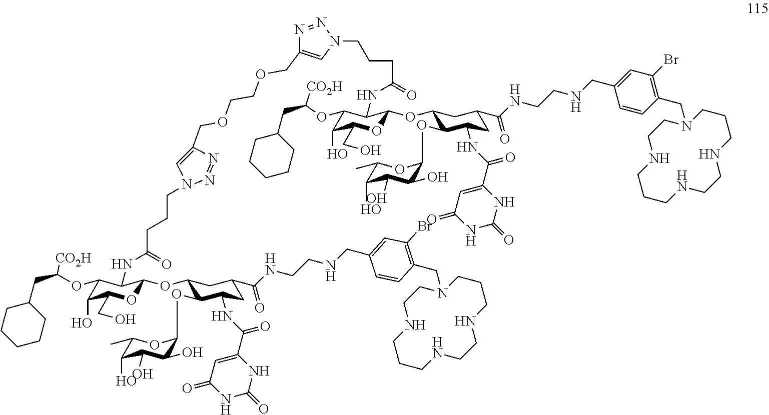

### Example 82

### Prophetic Synthesis of Multimeric Compound 116

Compound 116: Compound 116 can be prepared in an analogous fashion to FIG. 13 by replacing compound 22 with compound 50 in step a.

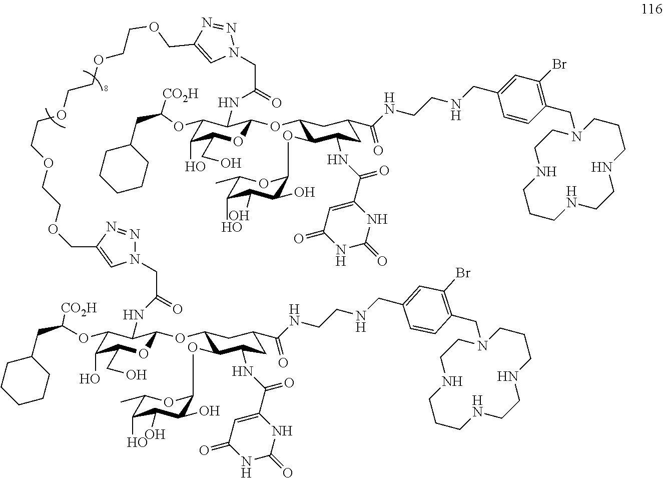

### Example 83

### Prophetic Synthesis of Multimeric Compound 117

Compound 117: Compound 117 can be prepared in an analogous fashion to FIG. 13 by replacing compound 22 with compound 51 in step a.

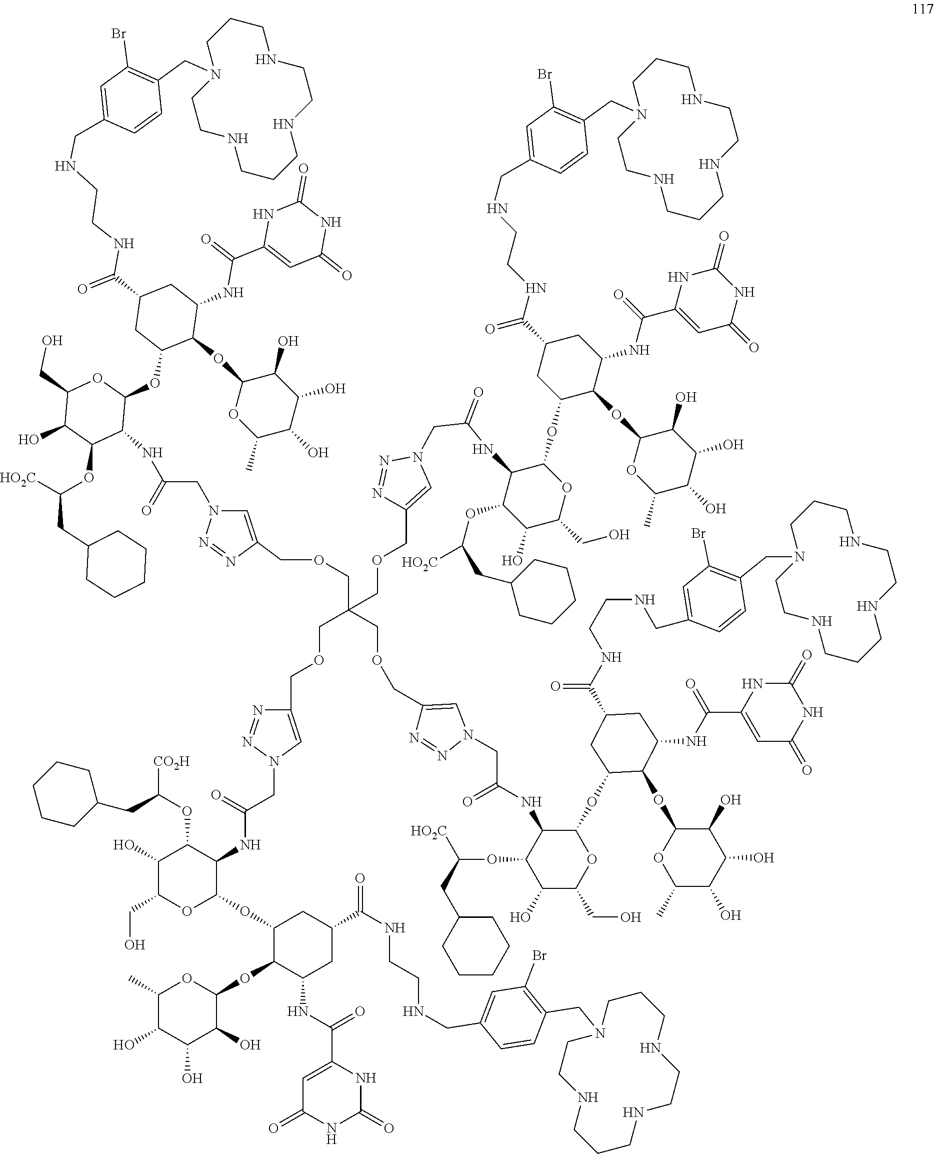

### Example 84

### Prophetic Synthesis of Multimeric Compound 118

Compound 118: Compound 118 can be prepared in an analogous fashion to FIG. 13 by replacing compound 22 with compound 52 in step a.

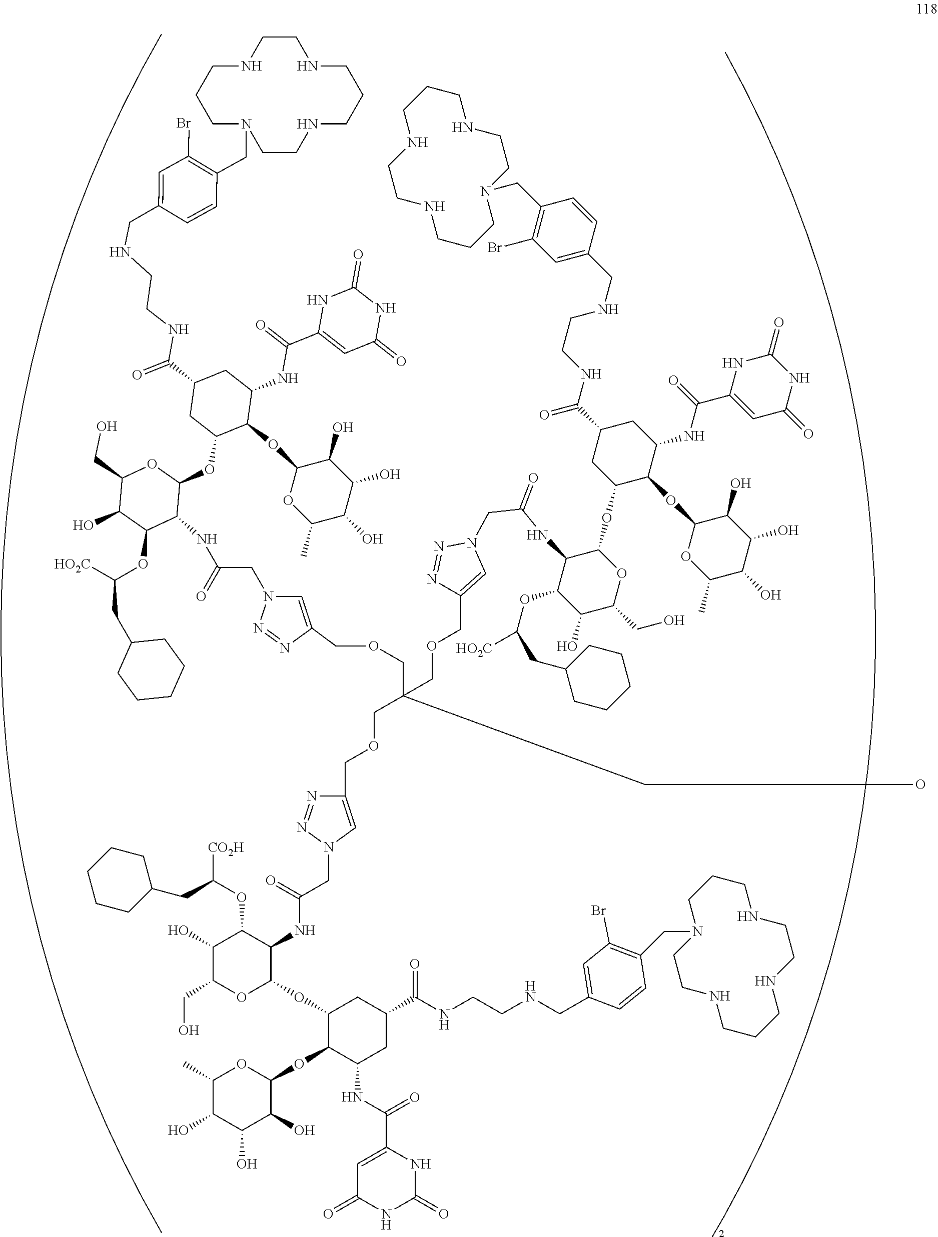

### Example 85

### Prophetic Synthesis of Multimeric Compound 119

Compound 119: Compound 119 can be prepared in an analogous fashion to FIG. 13 by replacing compound 22 with compound 53 in step a.

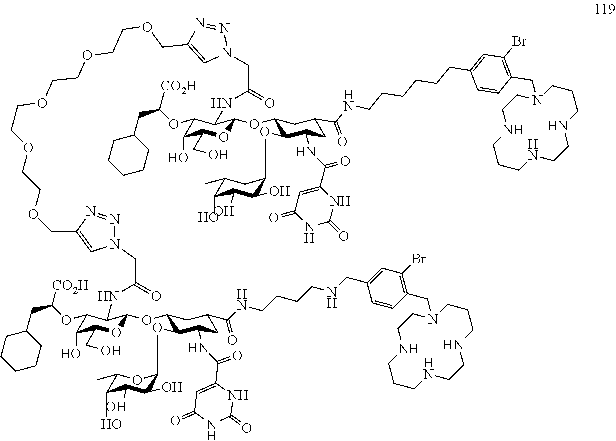

### Example 86

### Prophetic Synthesis of Multimeric Compound 120

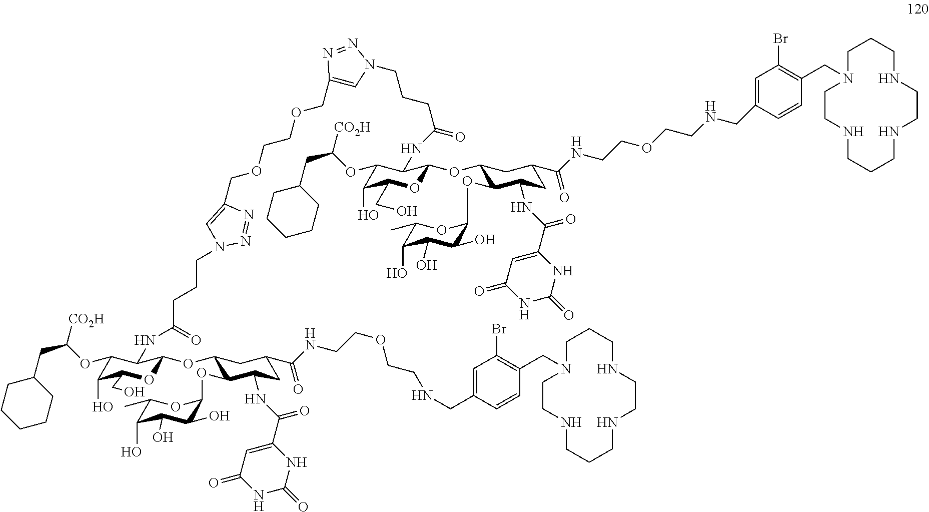

### Example 87

### Prophetic Synthesis of Multimeric Compound 121

Compound 121: Compound 121 can be prepared in an analogous fashion to FIG. 13 by replacing compound 22 with compound 56 in step a.

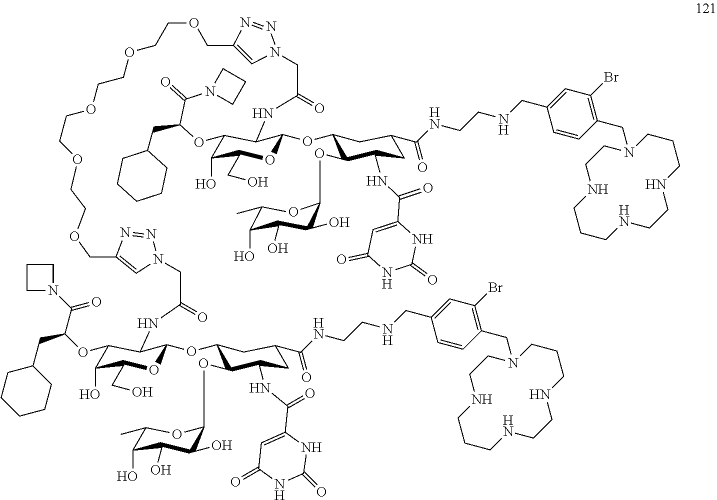

### Example 88

### Prophetic Synthesis of Multimeric Compound 122

Compound 122: Compound 122 can be prepared in an analogous fashion to FIG. 13 by replacing compound 22 with compound 57 in step a.

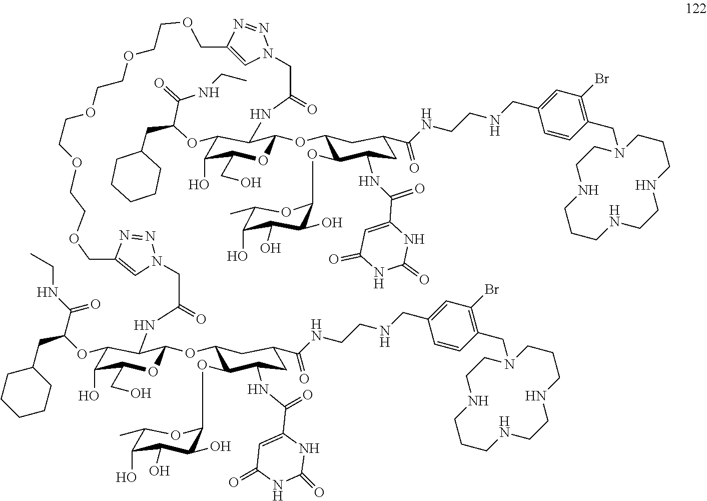

### Example 89

### Prophetic Synthesis of Multimeric Compound 123

Compound 123: Compound 123 can be prepared in an analogous fashion to FIG. 13 by replacing compound 22 with compound 58 in step a.

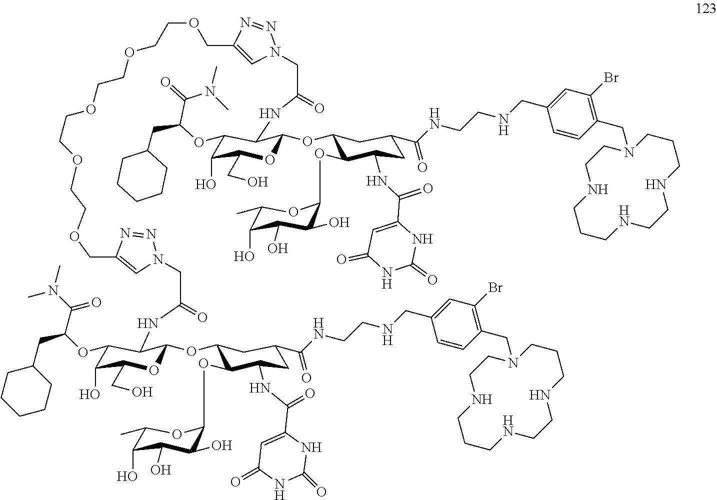

### Example 90

### Prophetic Synthesis of Multimeric Compound 124

Compound 124: Compound 124 can be prepared in an analogous fashion to FIG. 13 by replacing compound 22 with compound 59 in step a.

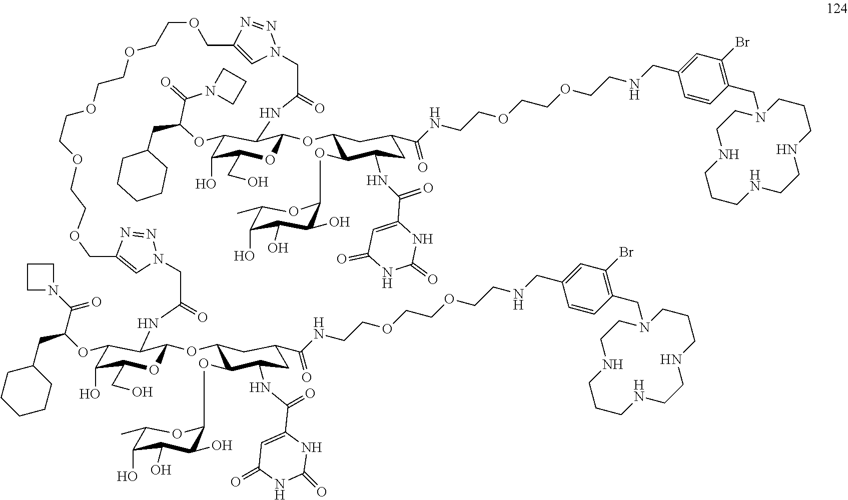

### Example 91

### Prophetic Synthesis of Multimeric Compound 125

Compound 125: Compound 125 can be prepared in an analogous fashion to FIG. 13 by replacing compound 22 with compound 68 in step a.

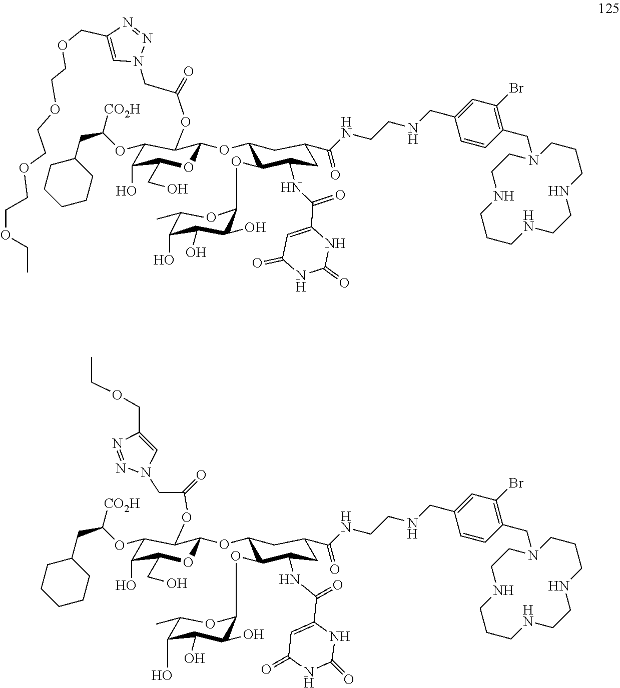

### Example 92

### Prophetic Synthesis of Multimeric Compound 126

Compound 126: Compound 126 can be prepared in an analogous fashion to FIG. 13 by replacing compound 22 with compound 69 in step a.

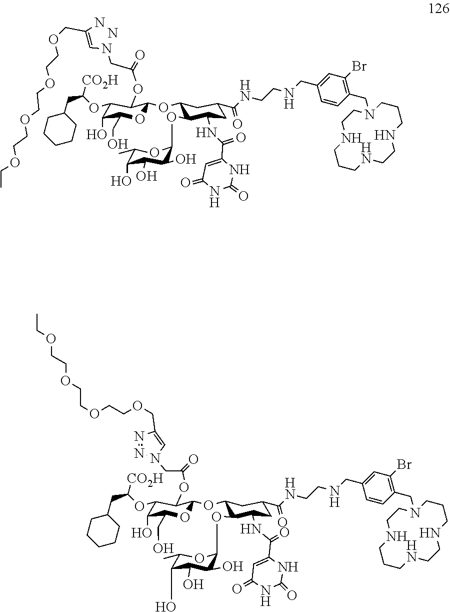

### Example 93

### Prophetic Synthesis of Multimeric Compound 127

Compound 127: Compound 127 can be prepared in an analogous fashion to FIG. 13 by replacing compound 22 with compound 70 in step a.

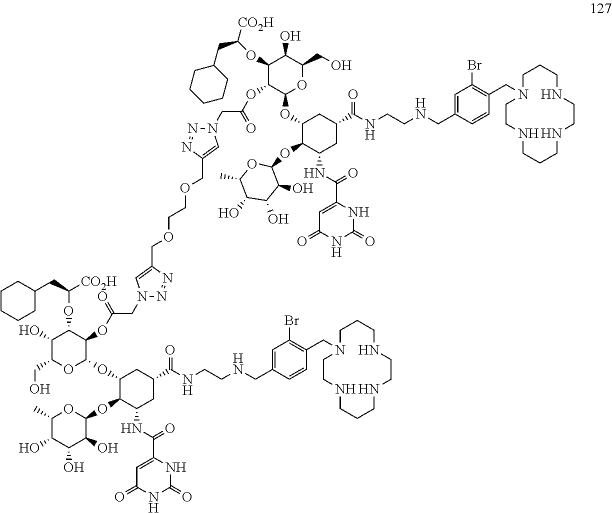 [SMILES: C[C@@H]1O[C@@H](O[C@@H]2[C@@H](NC(=O)c3cc(=O)[nH]c(=O)[nH]3)C[C@@H](C(=O)NCCNCc3ccc(CN4CCCNCCNCCCNCC4)c(Br)c3)C[C@H]2O[C@@H]2O[C@H](CO)[C@H](O)[C@H](O[C@@H](CC3CCCCC3)C(=O)O)[C@H]2OC(=O)Cn2cc(COCCOCc3cn(CC(=O)O[C@H]4[C@H](O[C@@H]5C[C@H](C(=O)NCCNCc6ccc(CN7CCCNCCNCCCNCC7)c(Br)c6)C[C@H](NC(=O)c6cc(=O)[nH]c(=O)[nH]6)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 94

### Prophetic Synthesis of Multimeric Compound 128

Compound 128: Compound 128 can be prepared in an analogous fashion to FIG. 13 by replacing compound 22 with compound 71 in step a.

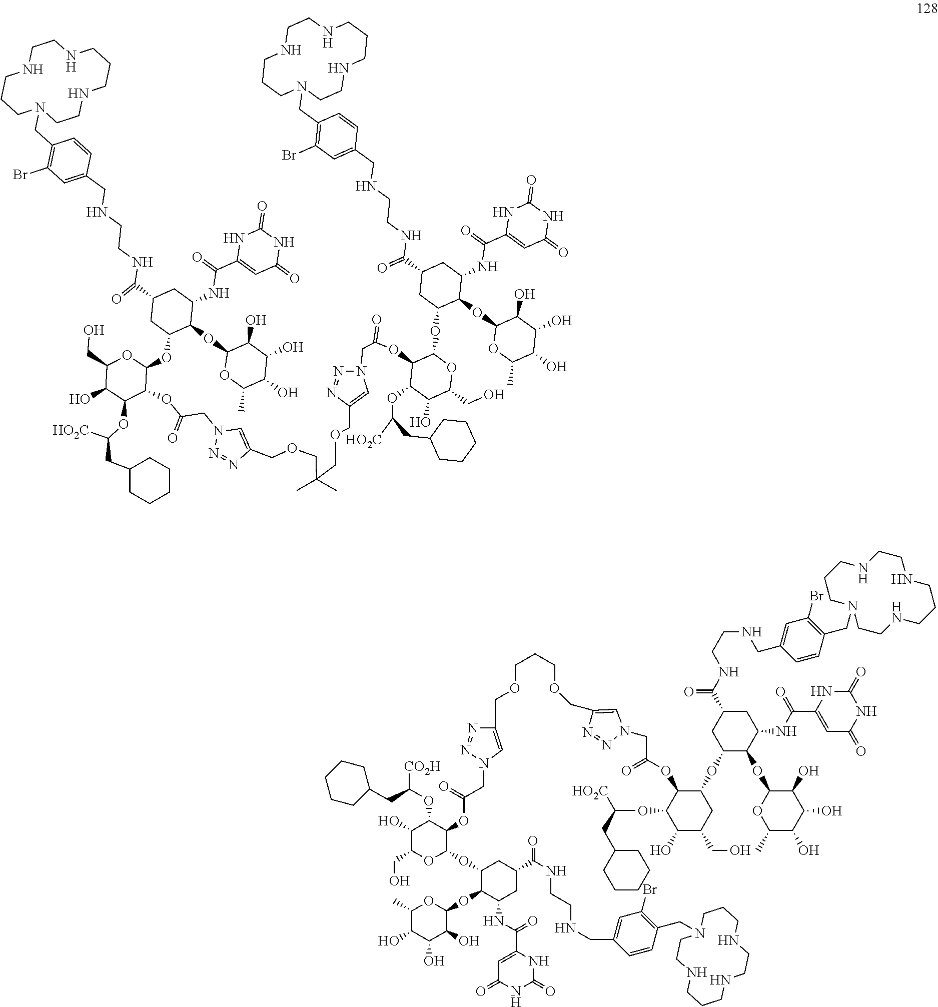

### Example 95

### Prophetic Synthesis of Multimeric Compound 129

Compound 129: Compound 129 can be prepared in an analogous fashion to FIG. 13 by replacing compound 22 with compound 73 in step a.

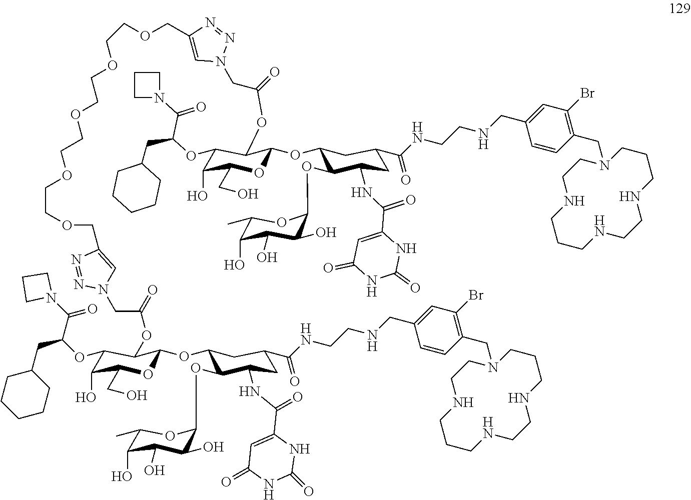

### Example 96

### Prophetic Synthesis of Multimeric Compound 130

Compound 130: Compound 130 can be prepared in an analogous fashion to FIG. 13 by replacing compound 22 with compound 78 in step a.

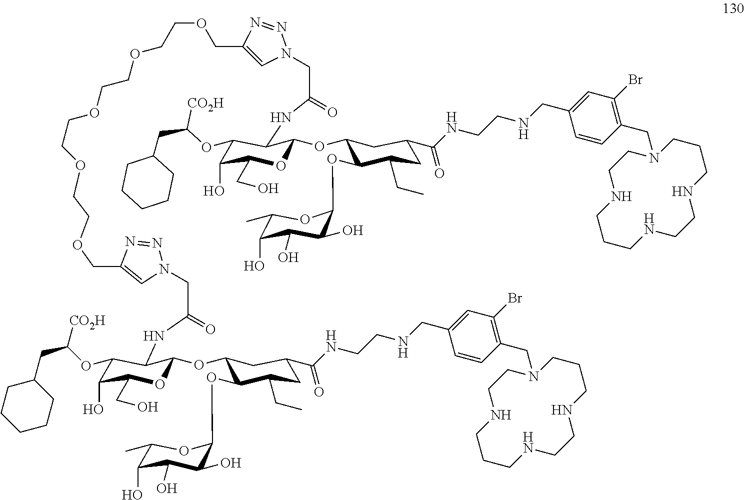

### Example 97

### Prophetic Synthesis of Multimeric Compound 131

Compound 131: Compound 131 can be prepared in an analogous fashion to FIG. 13 by replacing compound 22 with compound 79 in step a.

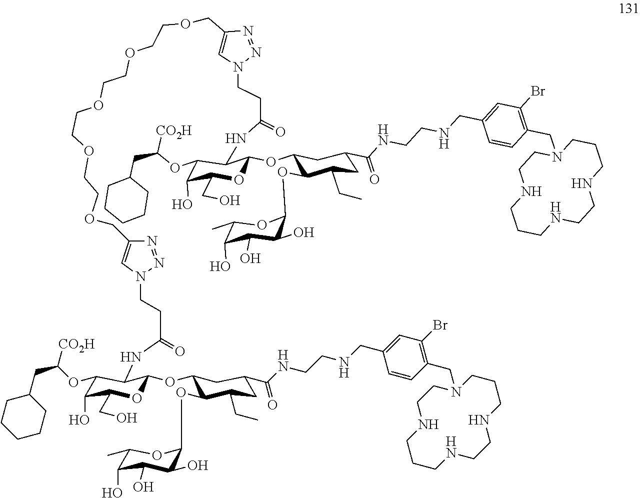

### Example 98

### Prophetic Synthesis of Multimeric Compound 132

Compound 132: Compound 132 can be prepared in an analogous fashion to FIG. 13 by replacing compound 22 with compound 80 in step a.

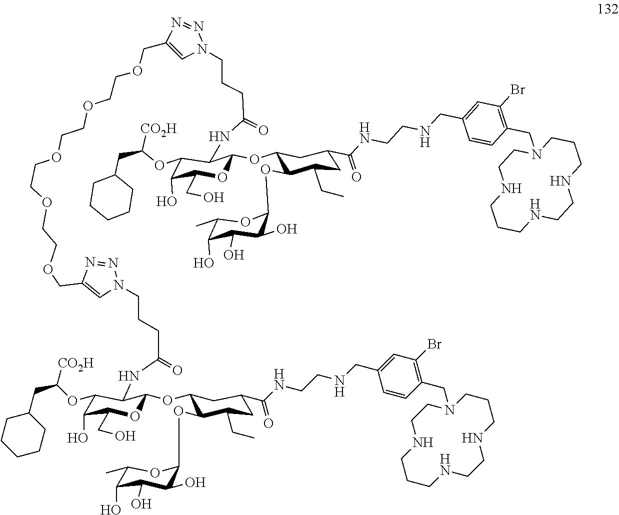

### Example 99

### Prophetic Synthesis of Multimeric Compound 133

Compound 133: Compound 133 can be prepared in an analogous fashion to FIG. 13 by replacing compound 22 with compound 81 in step a.

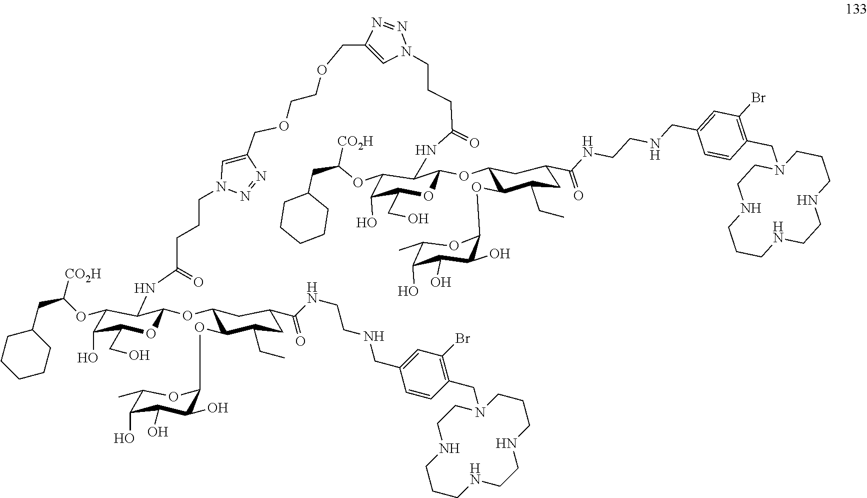

### Example 100

### Prophetic Synthesis of Multimeric Compound 134

Compound 134: Compound 134 can be prepared in an analogous fashion to FIG. 13 by replacing compound 22 with compound 82 in step a.

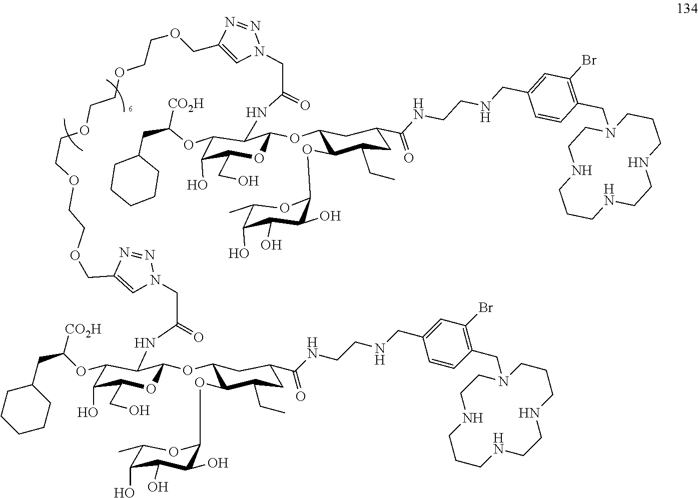

### Example 101

### Prophetic Synthesis of Multimeric Compound 135

Compound 135: Compound 135 can be prepared in an analogous fashion to FIG. 13 by replacing compound 22 with compound 83 in step a.

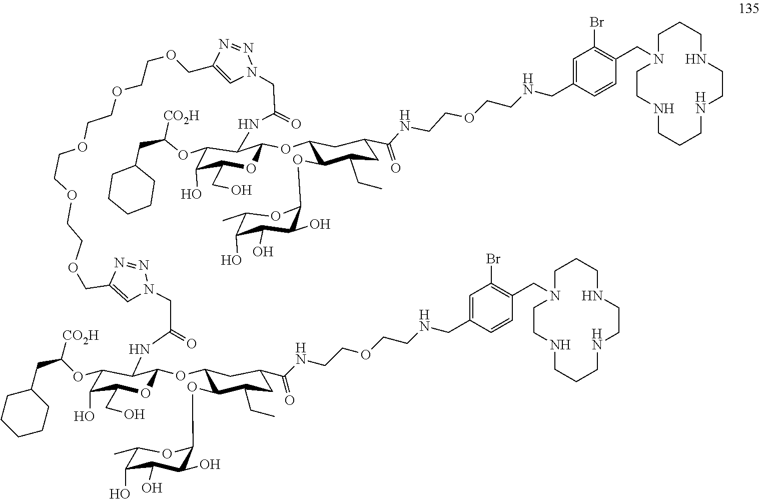

### Example 102

### Prophetic Synthesis of Multimeric Compound 136

Compound 136: Compound 136 can be prepared in an analogous fashion to FIG. 13 by replacing compound 22 with compound 84 in step a.

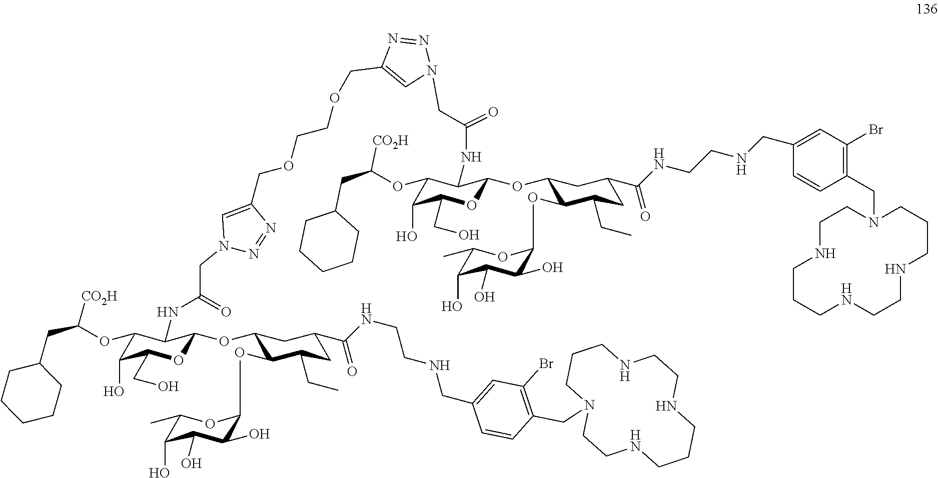

### Example 103

### Prophetic Synthesis of Multimeric Compound 137

Compound 137: Compound 137 can be prepared in an analogous fashion to FIG. 13 by replacing compound 22 with compound 85 in step a.

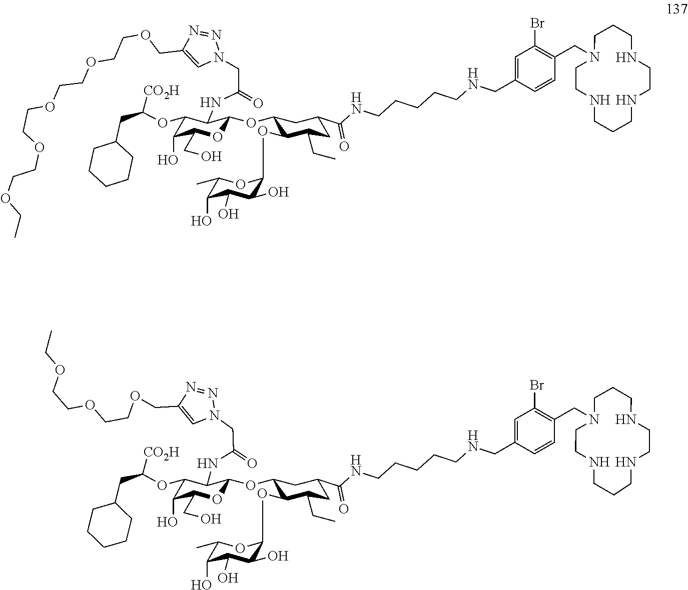

### Example 104

### Prophetic Synthesis of Multimeric Compound 138

Compound 138: Compound 138 can be prepared in an analogous fashion to FIG. 13 by replacing compound 22 with compound 87 in step a.

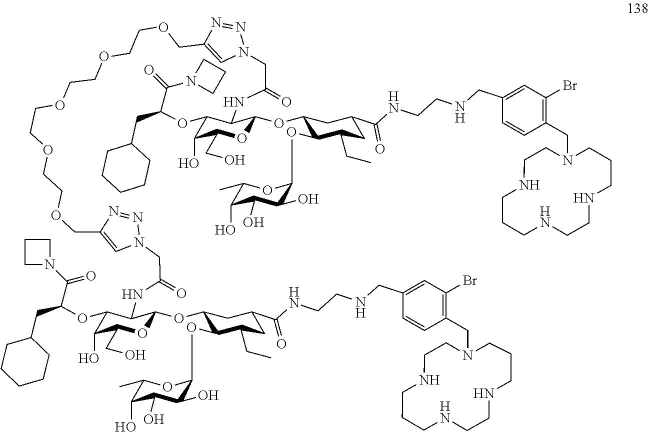

### Example 105

### Prophetic Synthesis of Multimeric Compound 139

Compound 139: Compound 139 can be prepared in an analogous fashion to FIG. 13 by replacing compound 22 with compound 88 in step a.

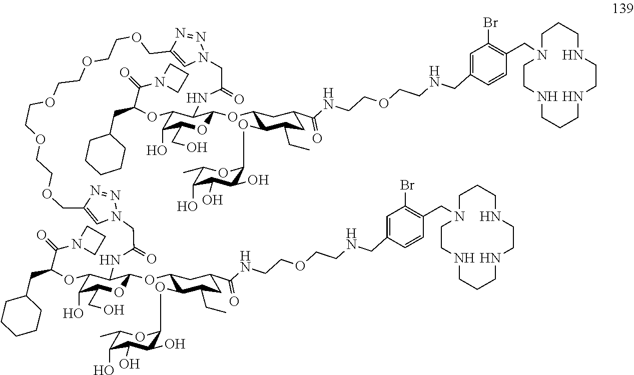

### Example 106

### Prophetic Synthesis of Multimeric Compound 140

Compound 140: Compound 140 can be prepared in an analogous fashion to FIG. 13 by replacing compound 22 with compound 89 in step a.

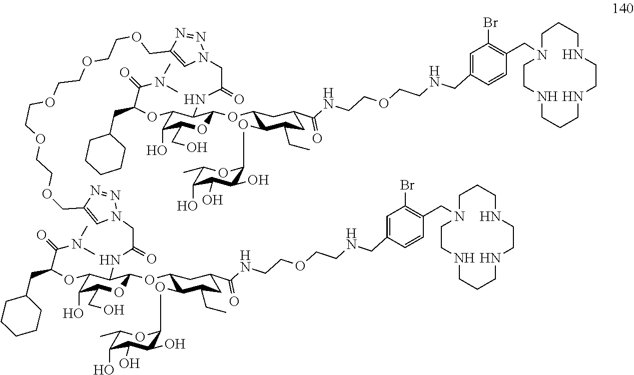

### Example 107

### Prophetic Synthesis of Multimeric Compound 141

Compound 141: Compound 141 can be prepared in an analogous fashion to FIG. 13 by replacing compound 22 with compound 90 in step a.

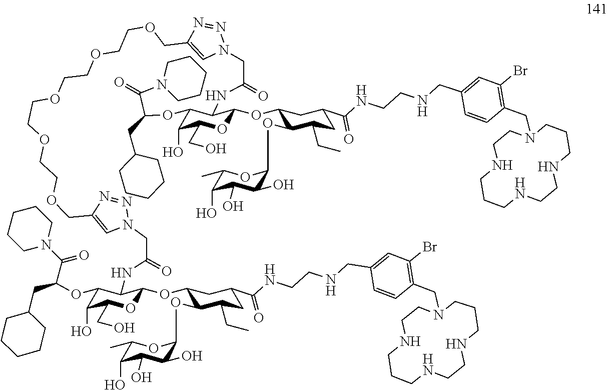

### Example 108

### Prophetic Synthesis of Multimeric Compound 142

Compound 142: Compound 142 can be prepared in an analogous fashion to FIG. 13 by replacing compound 22 with compound 91 in step a.

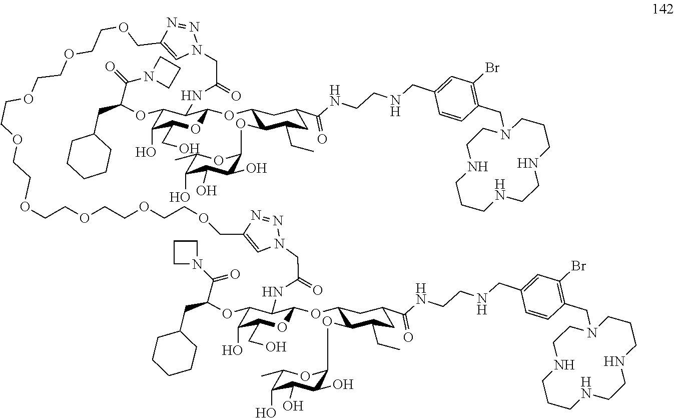

### Example 109

### Prophetic Synthesis of Multimeric Compound 143

Compound 143: Compound 143 can be prepared in an analogous fashion to FIG. 13 by replacing compound 22 with compound 92 in step a.

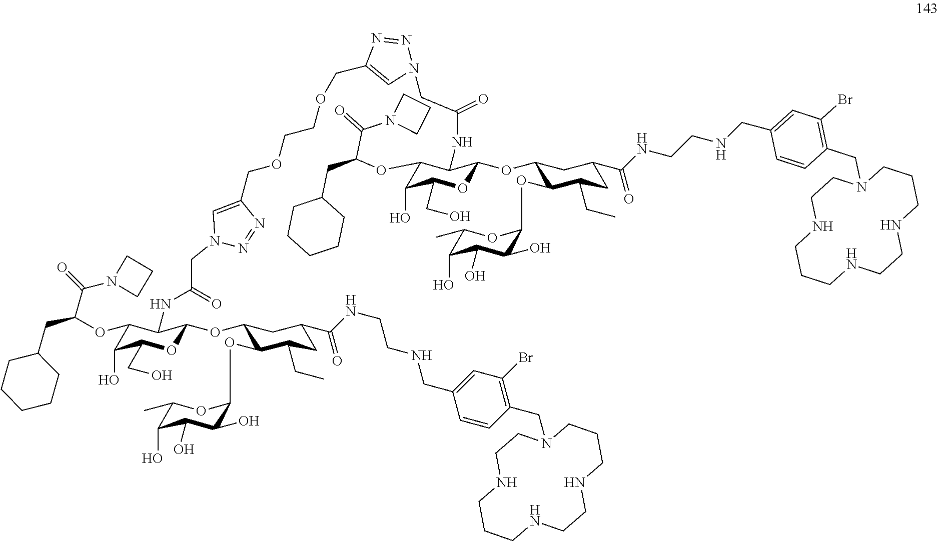

### Example 110

### Prophetic Synthesis of Multimeric Compound 144

Compound 144: Compound 144 can be prepared in an analogous fashion to FIG. 13 by replacing compound 22 with compound 93 in step a.

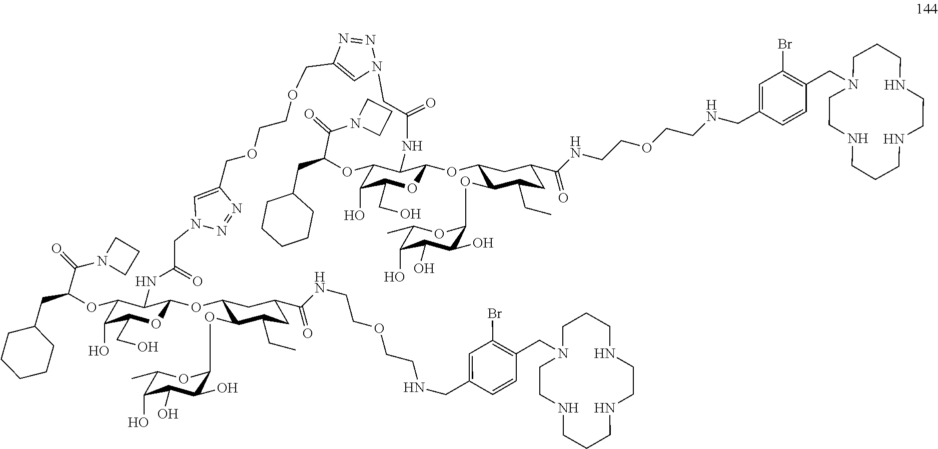

### Example 111

### Prophetic Synthesis of Multimeric Compound 146

Compound 315: To a solution of compound 314 (1 gm, 3.89 mmol) (preparation described in WO 2007/028050) and benzyl trichloroacetimidate (1.1 ml, 5.83 mmol) in anhydrous dichloromethane (10 ml) was added trimethylsilyl trifluoromethanesulfonate (70 uL, 0.4 mmol). The mixture was stirred at ambient temperature for 12 h. After this period the reaction was diluted with dichloromethane, washed with saturated NaHCO₃, dried over MgSO₄ and concentrated. The residue was purified by column chromatography to give compound 315 (0.8 gm, 60%).

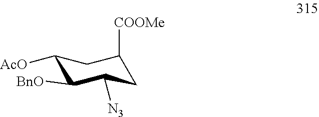

Compound 316: To a solution of compound 315 (800 mg, 2.3 mmol) in anhydrous methanol (1 ml) and anhydrous methyl acetate (5 ml) was added 0.5M sodium methoxide solution in methanol (9.2 ml). The mixture was stirred at 40° C. for 4 h. The reaction was quenched with acetic acid and concentrated. The residue was purified by column chromatography to afford compound 316 as mixture of epimers at the methyl ester with 75% equatorial and 25% axial epimer (242 mg, 35%).

¹H NMR (400 MHz, Chloroform-d) δ 7.48-7.32 (m, 6H), 4.97 (d, J=11.1 Hz, 1H), 4.72 (dd, J=11.1, 5.7 Hz, 1H), 3.77-3.65 (m, 6H), 3.22-3.15 (m, 1H), 2.92-2.82 (m, 1H), 2.39 (dddd, J=15.7, 10.6, 5.1, 2.7 Hz, 2H), 1.60 (dtd, J=13.9, 11.2, 5.4 Hz, 3H). MS: Calculated for C₁₅H₁₉N₃O₄=305.3, Found ES− positive m % z=306.1 (M+Na⁺).

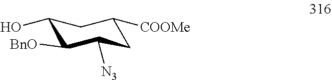

Compound 318: A solution of compound 317 (5 gm, 11.8 mmol) (preparation described in WO 2009/139719) in anhydrous methanol (20 ml) was treated with 0.5 M solution of sodium methoxide in methanol (5 ml) for 3 h. Solvent was removed in vacuo and the residue was co-evaporated with toluene (20 ml) three times. The residue was dissolved in pyridine (20 ml) followed by addition of benzoyl chloride (4.1 ml, 35.4 mmol) over 10 minutes. The reaction mixture was stirred at ambient temperature under an atmosphere of argon for 22 h. The reaction mixture was concentrated to dryness, dissolved in dichloromethane, washed with cold 1N hydrochloric acid and cold water, dried over MgSO₄, filtered, and concentrated. The residue was purified by column chromatography to give compound 318. MS: Calculated for C₃₃H₂₇N₃O₇S=609.2, Found ES− positive m/z=610.2 (M+Na⁺).

Compound 319: A mixture of compound 318 (2.4 gm, 3.93 mmol), diphenyl sulfoxide (1.5 gm, 7.3 mmol) and 2,6-di-tert-butyl pyridine (1.8 gm, 7.8 mmol) was dissolved in anhydrous dichloromethane (10 ml) at room temperature. The reaction mixture was cooled to −60° C. Triflic anhydride (0.62 ml, 3.67 mmol) was added dropwise and the mixture was stirred for 15 minutes at the same temperature. A solution of compound 316 (0.8 gm, 2.6 mmol) in anhydrous dichloromethane (10 ml) was added dropwise to the reaction mixture. The mixture was allowed to warm to 0° C. over 2 h. The reaction mixture was diluted with dichloromethane, transferred to a separatory funnel and washed with saturated sodium bicarbonate solution followed by brine. The organic phase was dried over MgSO₄, filtered, and concentrated. The residue was separated by column chromatography to afford compound 319 as a white solid (1.2 gm, 57%). MS: Calculated for C₄₂H₄₀N₆O₁₁=804.3, Found ES-positive m/z=805.3 (M+Na⁺).

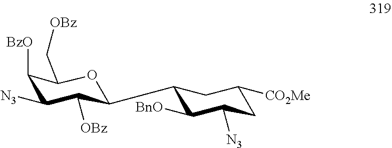

Compound 320: To a solution of compound 319 (1.2 gm 2.067 mmol) and 2-fluorophenyl acetylene (1.2 ml, 10.3 mmol) in methanol (30 ml) was added a stock solution of copper sulfate and tris(3-hydroxypropyltriazolylmethyl) amine in water (2.58 ml). The reaction was initiated by addition of an aqueous solution of sodium ascorbate (0.9 gm, 4.5 mmol) and the mixture was stirred at ambient temperature for 16 hours. The mixture was co-evaporated with dry silica gel and purified by column chromatography to afford compound 320 as a white solid (1.2 gm, 77%).

Stock solution of Copper Sulfate/THPTA—(100 mg of copper sulfate pentahydrate and 200 mg of tris(3-hydroxypropyltriazolylmethyl)amine were dissolved in 10 ml of water).

¹H NMR (400 MHz, Chloroform-d) δ 8.07-8.00 (m, 2H), 7.96 (ddd, J=9.8, 8.2, 1.3 Hz, 4H), 7.79 (d, J=5.4 Hz, 2H), 7.65-7.53 (m, 5H), 7.43 (ddt, J=22.4, 10.7, 5.0 Hz, 7H), 7.25-7.01 (m, 9H), 6.92 (td, J=7.6, 7.1, 2.2 Hz, 1H), 6.13-6.02 (m, 2H), 5.58 (dd, J=11.6, 3.2 Hz, 1H), 5.15 (d, J=7.5 Hz, 1H), 4.98 (d, J=10.3 Hz, 1H), 4.68 (dd, J=11.2, 5.7 Hz, 1H), 4.52 (dq, J=22.1, 6.6, 5.6 Hz, 2H), 4.35 (dd, J=11.1, 7.6 Hz, 1H), 4.28-4.18 (m, 1H), 4.11 (d, J=10.3 Hz, 1H), 3.87 (t, J=9.1 Hz, 1H), 3.71 (s, 3H), 2.95 (s, 1H), 2.62-2.43 (m, 3H), 1.55 (dt, J=12.7, 6.1 Hz, 1H). MS: Calculated for C₅₈H₅₀N₆O₁₁=1044.4, Found ES-positive m/z=1045.5 (M+Na⁺).

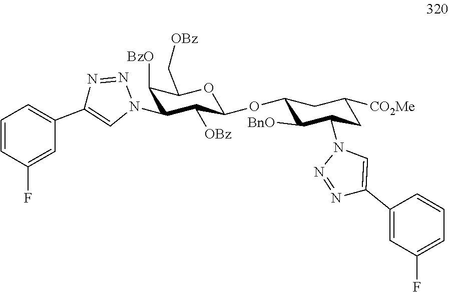

Compound 145: To a solution of compound 320 (1.2 gm, 1.1 mmol) in iso-propanol (40 ml) was added Na-metal (80 mg, 3.4 mmol) at ambient temperature and the mixture was stirred for 12 hours at 50° C. 10% aqueous sodium hydroxide (2 ml) was added to the reaction mixture and stirring continued for another 6 hours at 50° C. The reaction mixture was cooled to room temperature and neutralized with 50% aqueous hydrochloric acid, To the mixture was added 10% Pd(OH)₂ on carbon (0.6 gm) and the reaction mixture was stirred under an atmosphere of hydrogen for 12 hours. The reaction mixture was filtered through a Celite pad and concentrated. The residue was separated by HPLC to give compound 145 as a white solid (0.5 gm, 70%). HPLC Conditions—Waters preparative HPLC system was used with ELSD & PDA detectors. Kinetex XB—C18, 100 A, 5 uM, 250×21.2 mm column (from Phenomenex) was used with 0.2% formic acid in water as solvent A and acetonitrile as solvent B at a flow rate of 20 mL/min.

¹H NMR (400 MHz, DMSO-d6) δ 8.77 (s, 1H), 8.68 (s, 1H), 7.77-7.60 (m, 5H), 7.49 (tdd, J=8.3, 6.1, 2.6 Hz, 3H), 7.15 (tt, J=8.6, 3.2 Hz, 3H), 4.83 (dd, J=10.9, 3.1 Hz, 1H), 4.63 (d, J=7.5 Hz, 1H), 4.53-4.41 (m, 1H), 4.10 (dd, J=10.9, 7.5 Hz, 1H), 3.92 (d, J=3.2 Hz, 1H), 3.74 (h, J=6.0, 5.6 Hz, 3H), 3.65-3.24 (m, 5H), 2.37 (d, J=13.4 Hz, 1H), 2.24-2.04 (m, 2H), 1.93 (q, J=12.5 Hz, 1H), 1.46 (t, J=12.1 Hz, 1H). MS: Calculated for C₂₉H₃₀F₂N₆O₈=628.2, Found ES− positive m/z=629.2 (M+Na⁺)

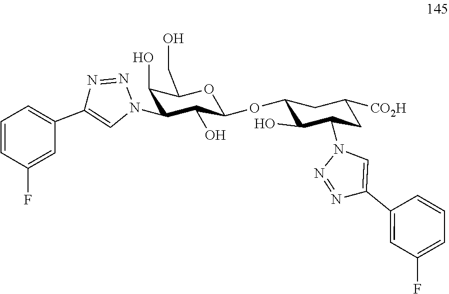

Compound 146: To a solution of compound 145 (3 eq) in anhydrous DMF was added HATU (3.3 eq) and DIPEA (5 eq). The mixture was stirred at ambient temperature for 15 minutes followed by addition of compound 22 (1 eq). The mixture was stirred at ambient temperature for 12 h. The solvent was removed in vacuo and the residue was purified by HPLC to afford compound 146.

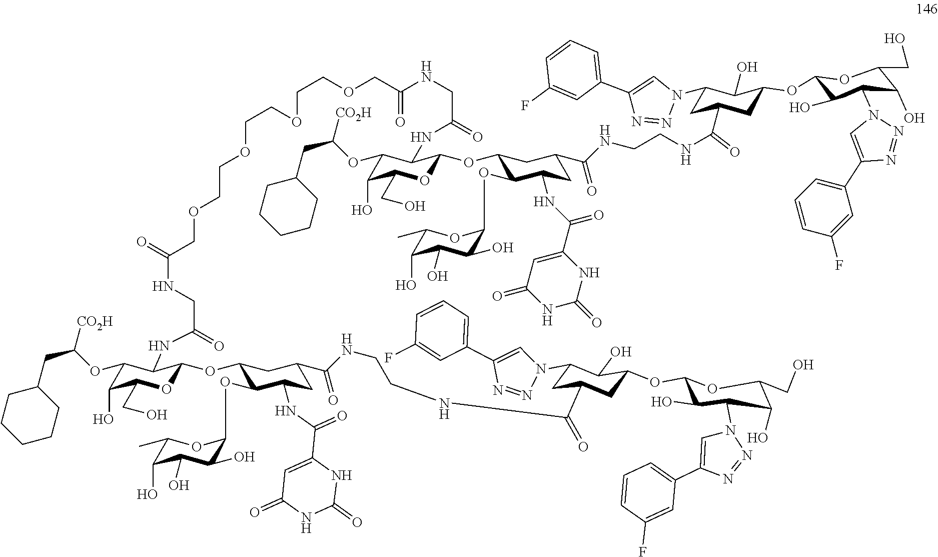

### Example 112

### Prophetic Synthesis of Multimeric Compound 147

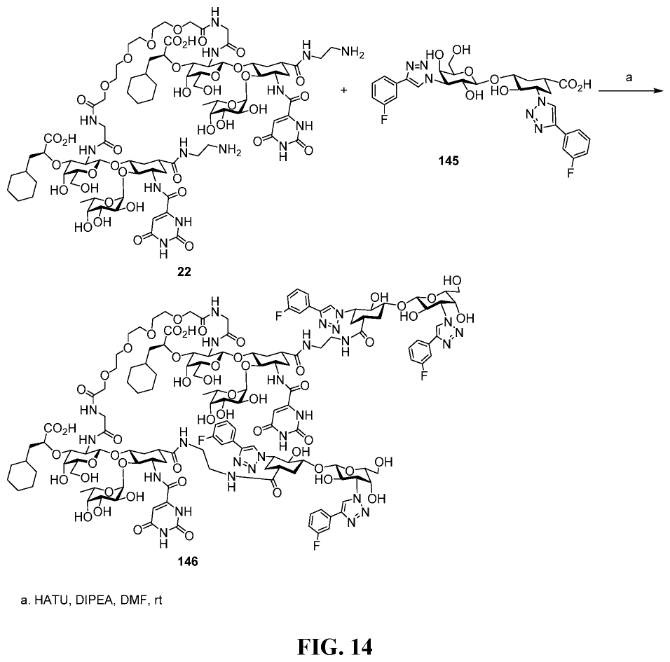

Compound 147: Compound 147 can be prepared in an analogous fashion to FIG. 14 by replacing compound 22 with compound 23.

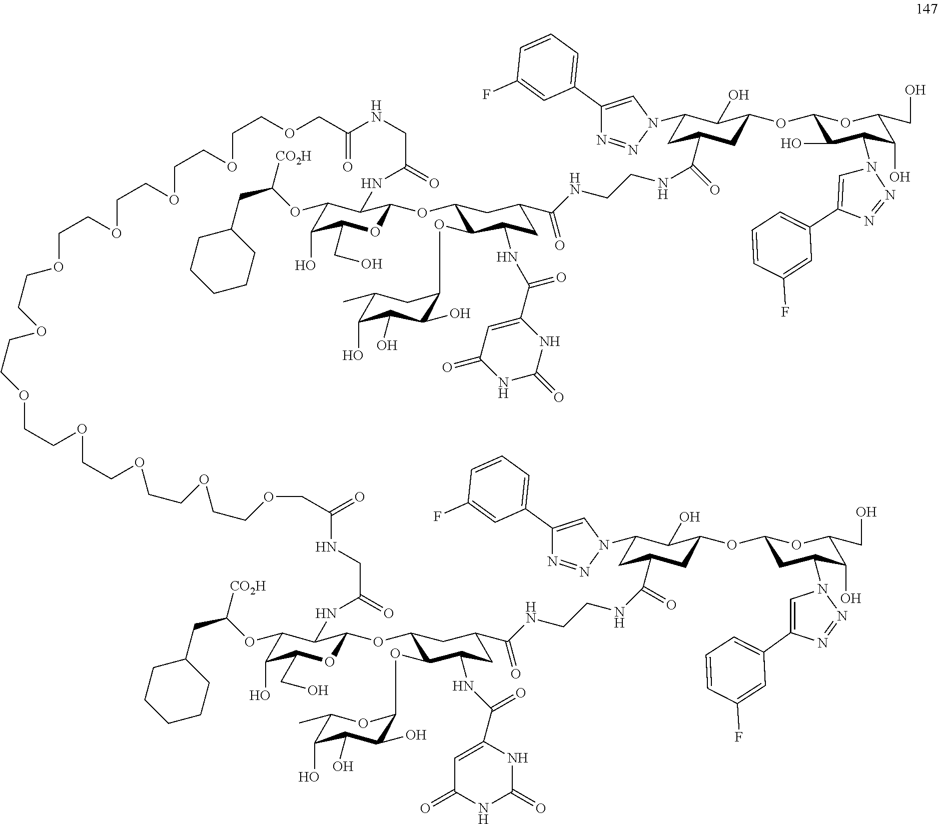

### Example 113

### Prophetic Synthesis of Multimeric Compound 148

Compound 148: Compound 148 can be prepared in an analogous fashion to FIG. 14 by replacing compound 22 with compound 24.

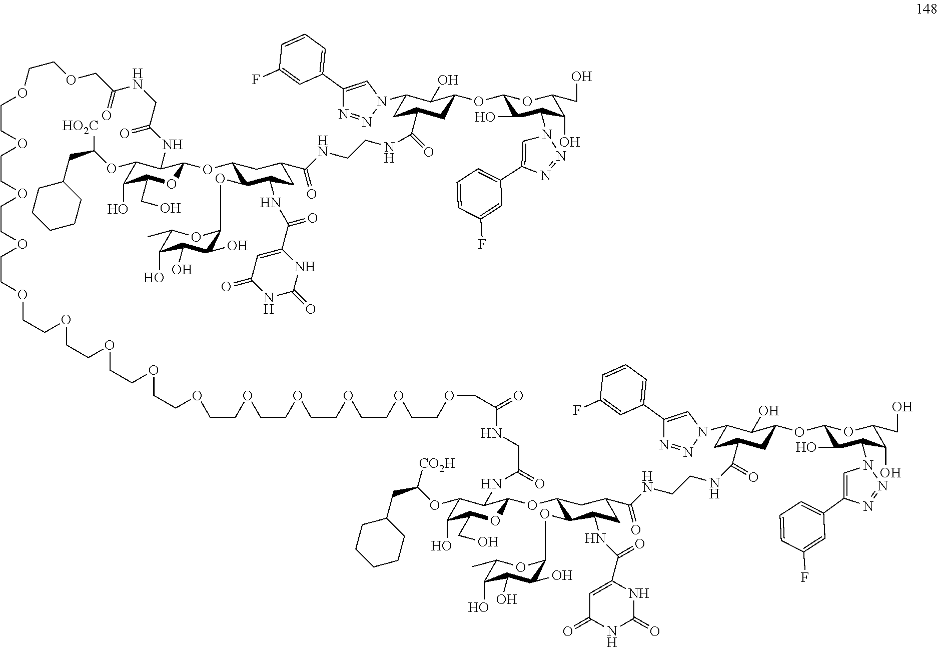

### Example 114

### Prophetic Synthesis of Multimeric Compound 149

Compound 149: Compound 149 can be prepared in an analogous fashion to FIG. 14 by replacing compound 22 with compound 25.

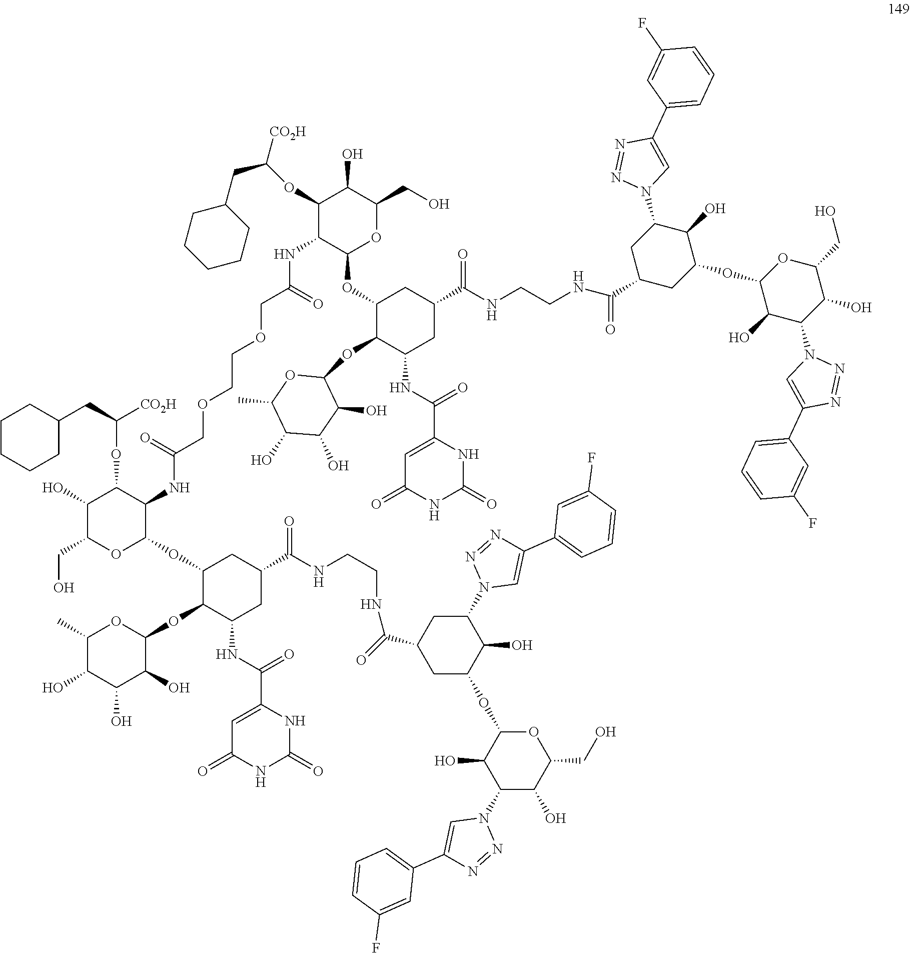

### Example 115

### Prophetic Synthesis of Multimeric Compound 150

Compound 150: Compound 150 can be prepared in an analogous fashion to FIG. 14 by replacing compound 22 with compound 26.

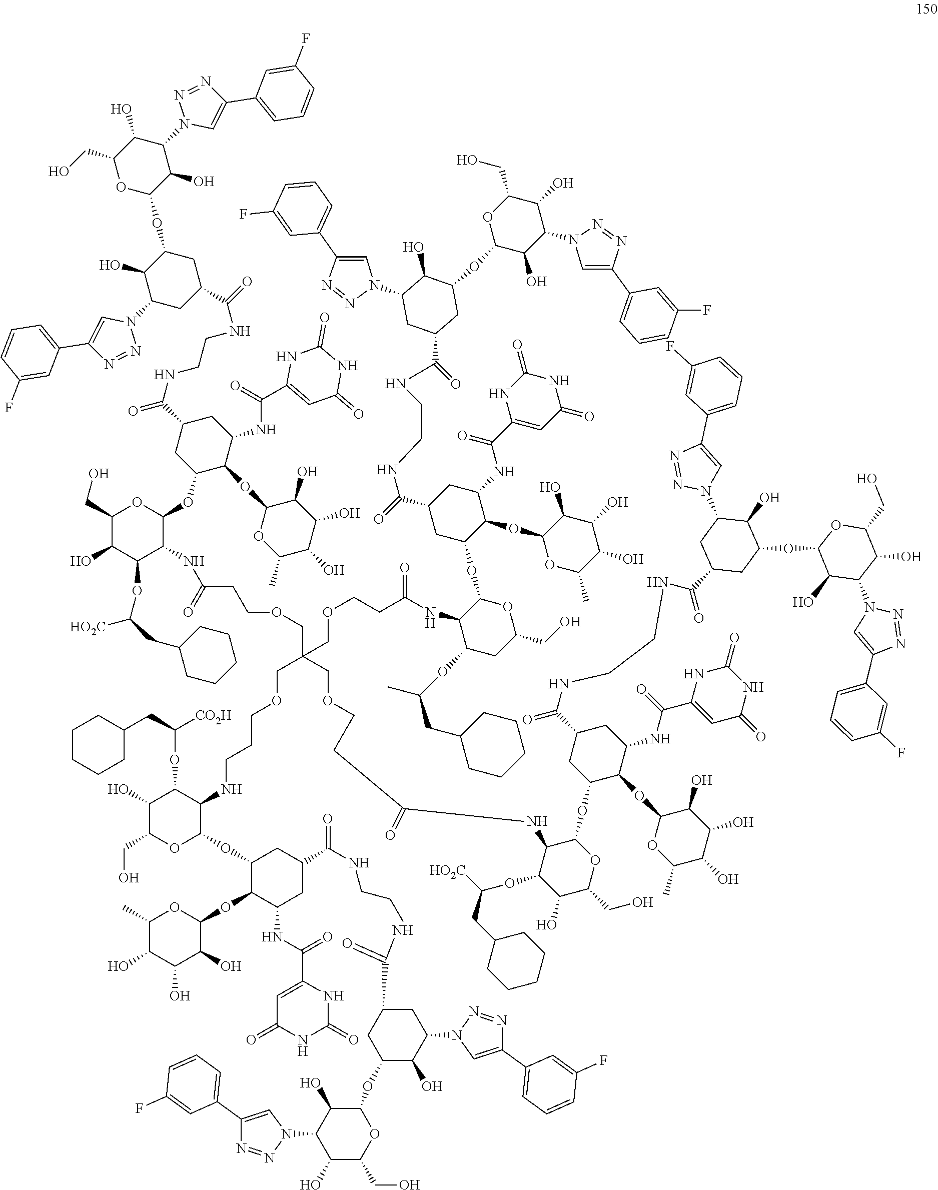

### Example 116

### Prophetic Synthesis of Multimeric Compound 151

Compound 151: Compound 151 can be prepared in an analogous fashion to FIG. 14 by replacing compound 22 with compound 27.

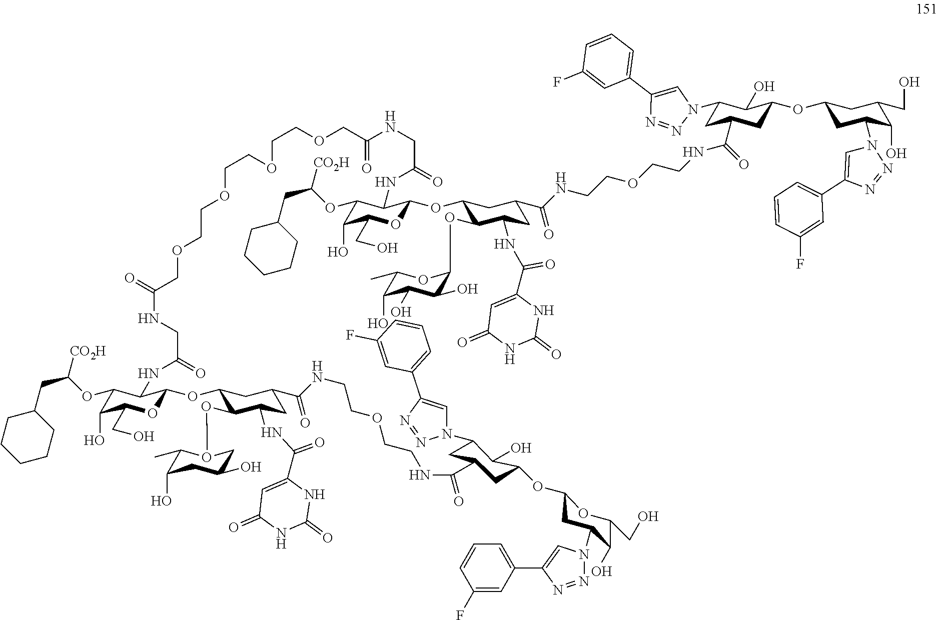

### Example 117

### Prophetic Synthesis of Multimeric Compound 152

Compound 152: Compound 152 can be prepared in an analogous fashion to FIG. 14 by replacing compound 22 with compound 28.

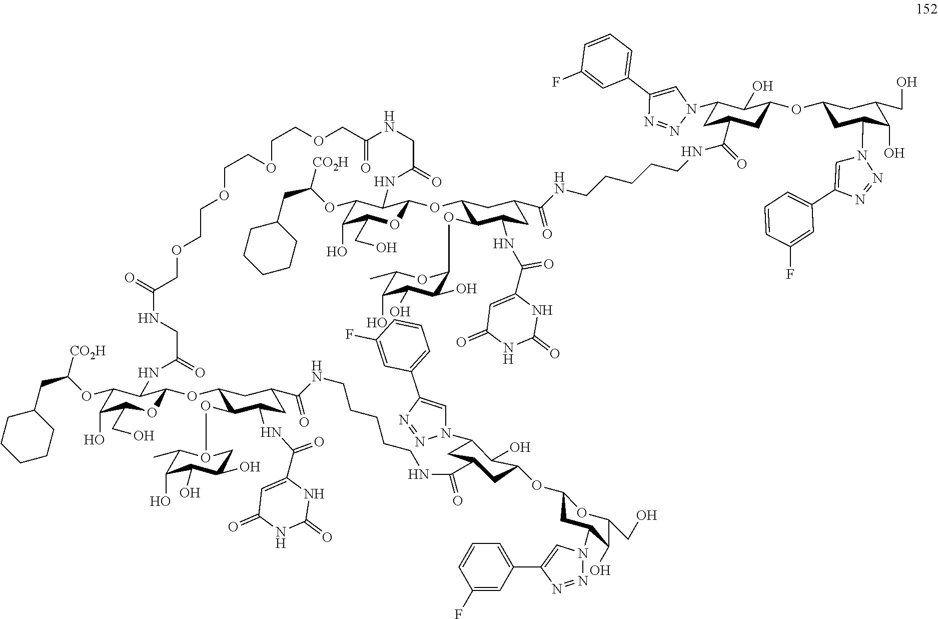

### Example 118

### Prophetic Synthesis of Multimeric Compound 153

Compound 153: Compound 153 can be prepared in an analogous fashion to FIG. 14 by replacing compound 22 with compound 29.

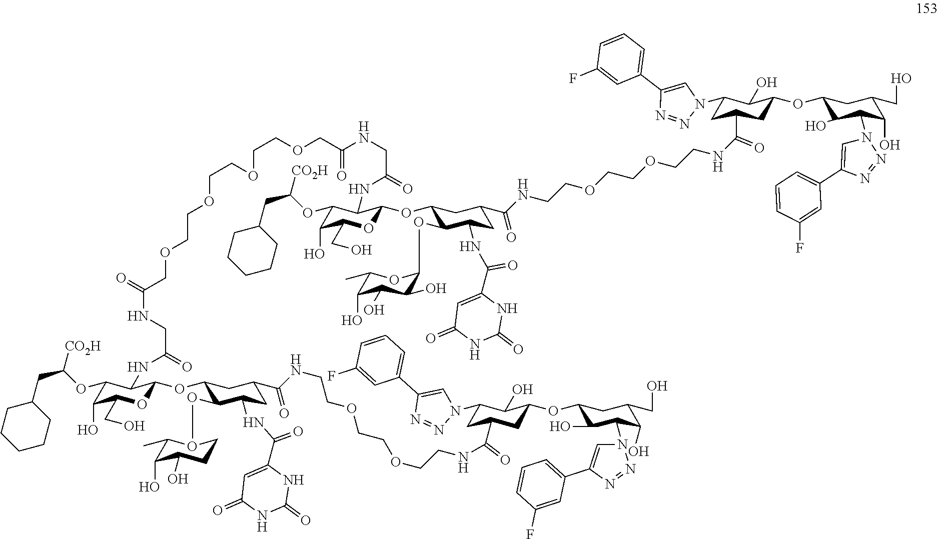

### Example 119

### Prophetic Synthesis of Multimeric Compound 154

Compound 154: Compound 154 can be prepared in an analogous fashion to FIG. 14 by replacing compound 22 with compound 30.

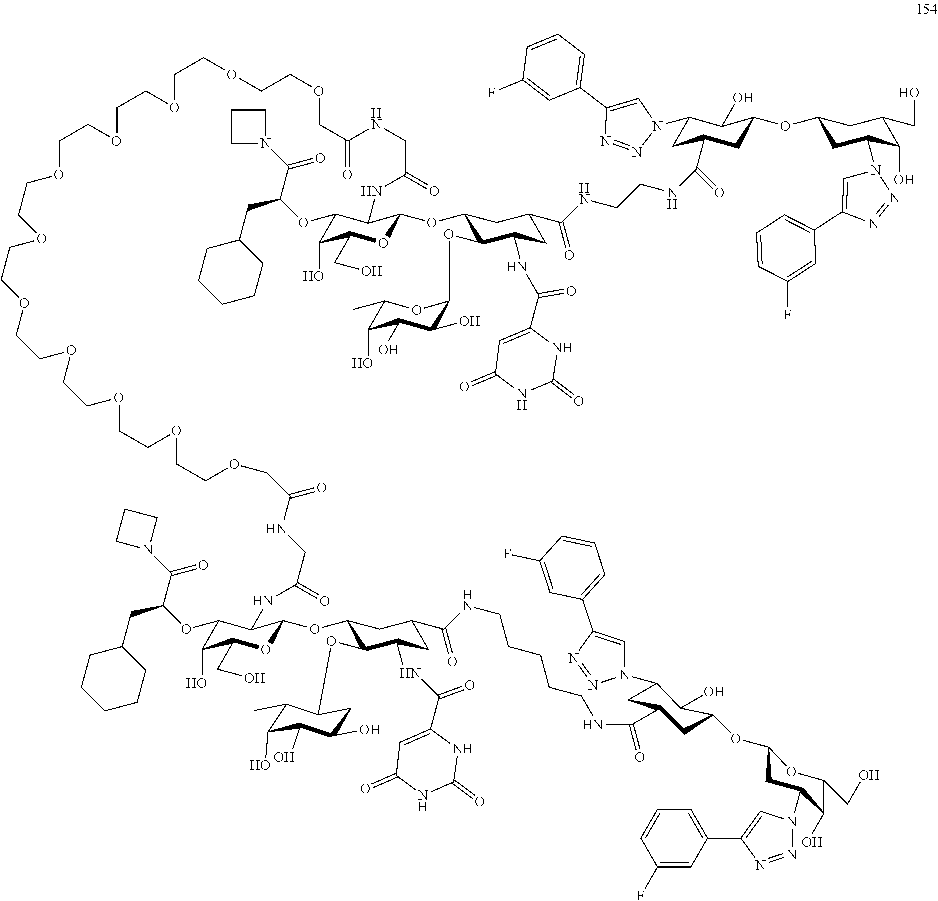

### Example 120

### Prophetic Synthesis of Multimeric Compound 155

Compound 155: Compound 155 can be prepared in an analogous fashion to FIG. 14 by replacing compound 22 with compound 31.

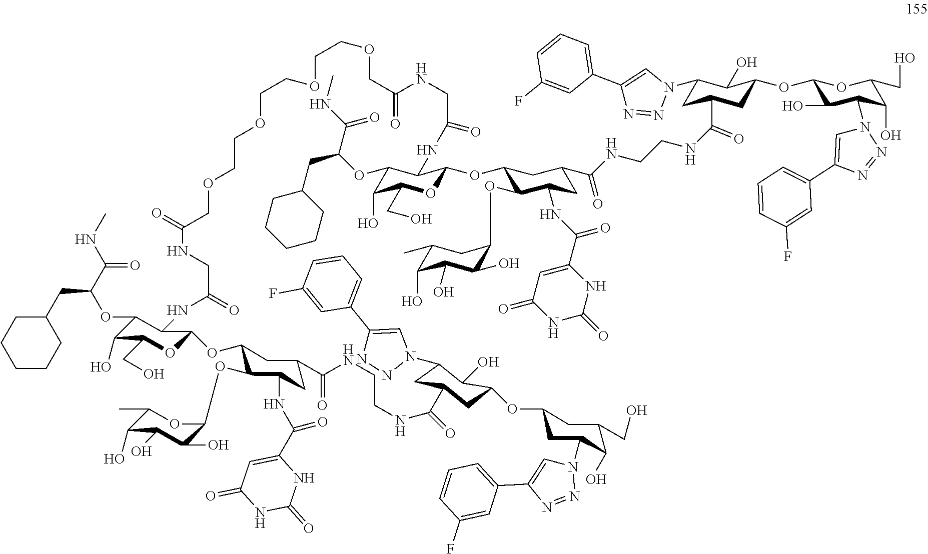

### Example 121

### Prophetic Synthesis of Multimeric Compound 156

Compound 156: Compound 156 can be prepared in an analogous fashion to FIG. 14 by replacing compound 22 with compound 32.

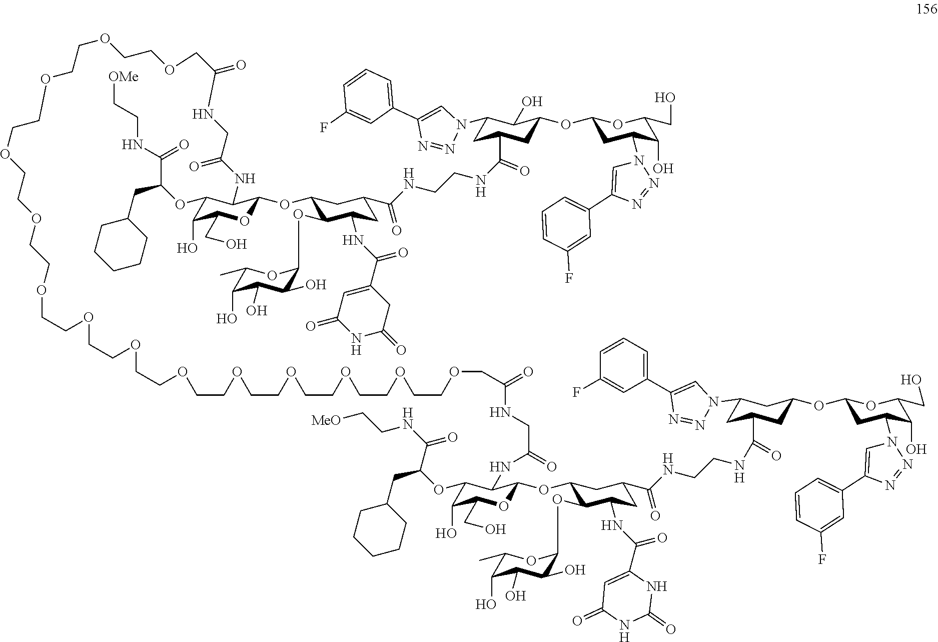

### Example 122

### Prophetic Synthesis of Multimeric Compound 157

Compound 157: Compound 157 can be prepared in an analogous fashion to FIG. 14 by replacing compound 22 with compound 33.

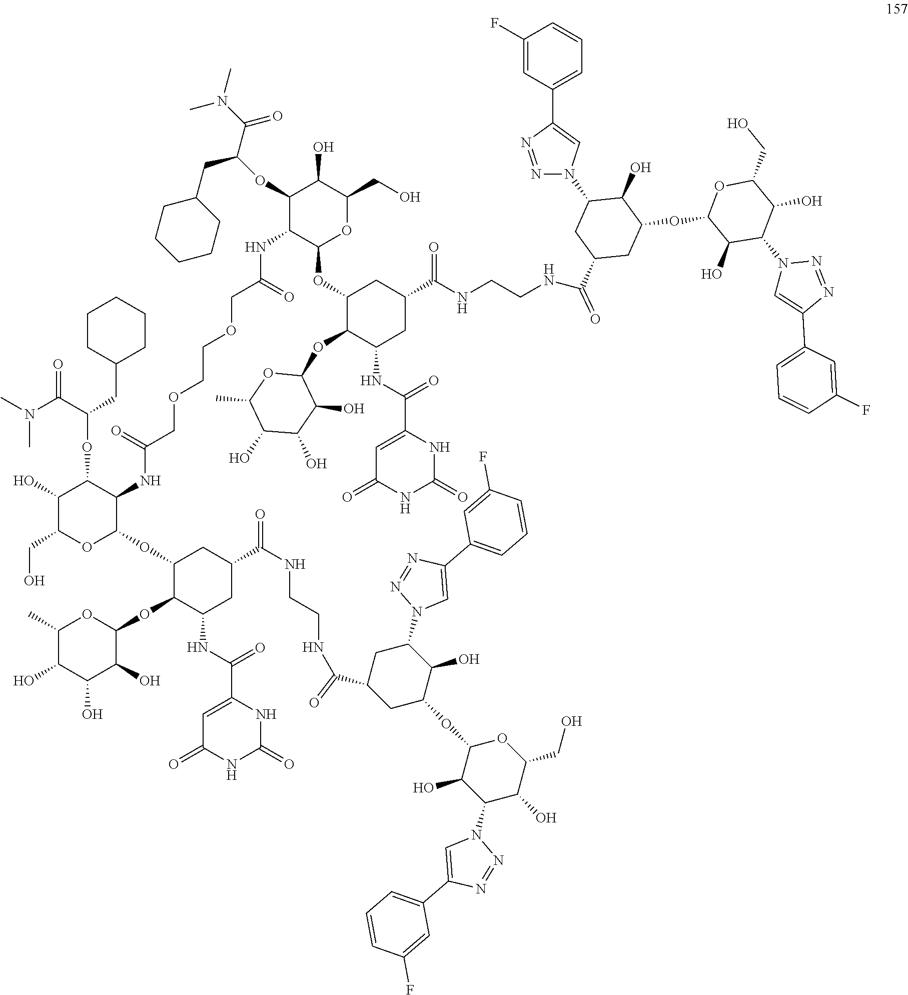

Compound 158: Compound 158 can be prepared in an analogous fashion to FIG. 14 by replacing compound 22 with compound 34.

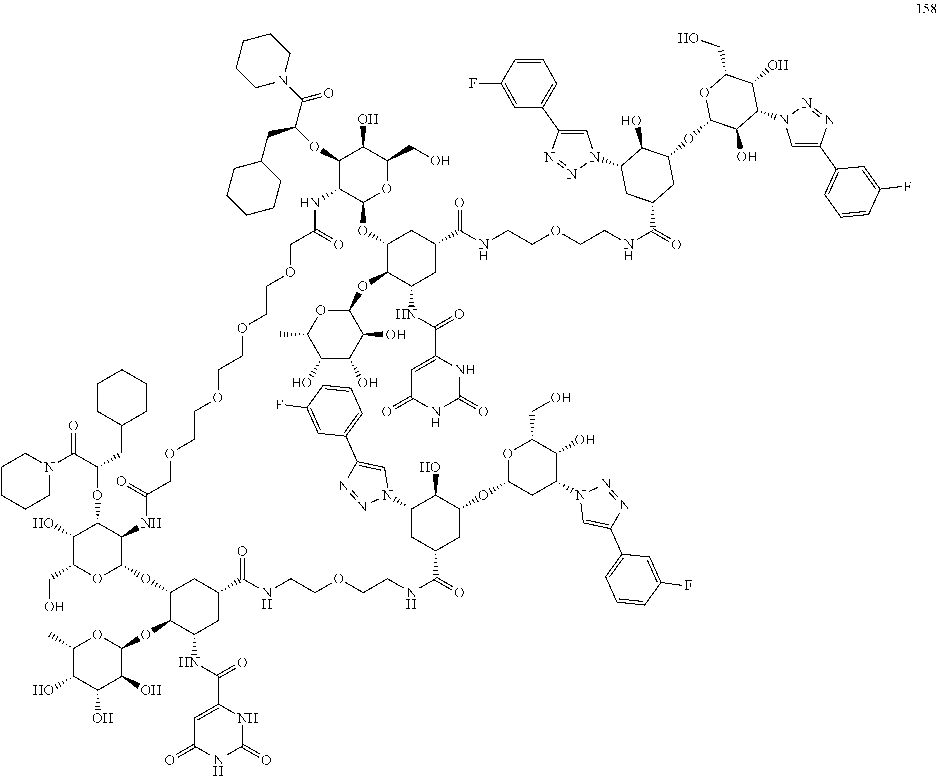

### Example 124

### Prophetic Synthesis of Multimeric Compound 159

Compound 159: Compound 159 can be prepared in an analogous fashion to FIG. 14 by replacing compound 22 with compound 37.

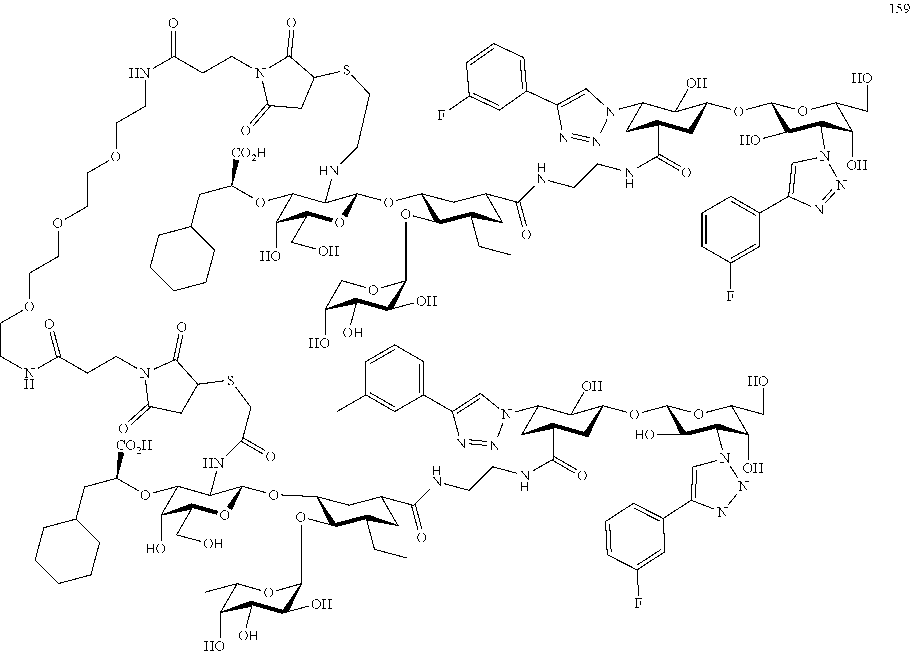

### Example 125

### Prophetic Synthesis of Multimeric Compound 160

Compound 160: Compound 160 can be prepared in an analogous fashion to FIG. 14 by replacing compound 22 with compound 38.

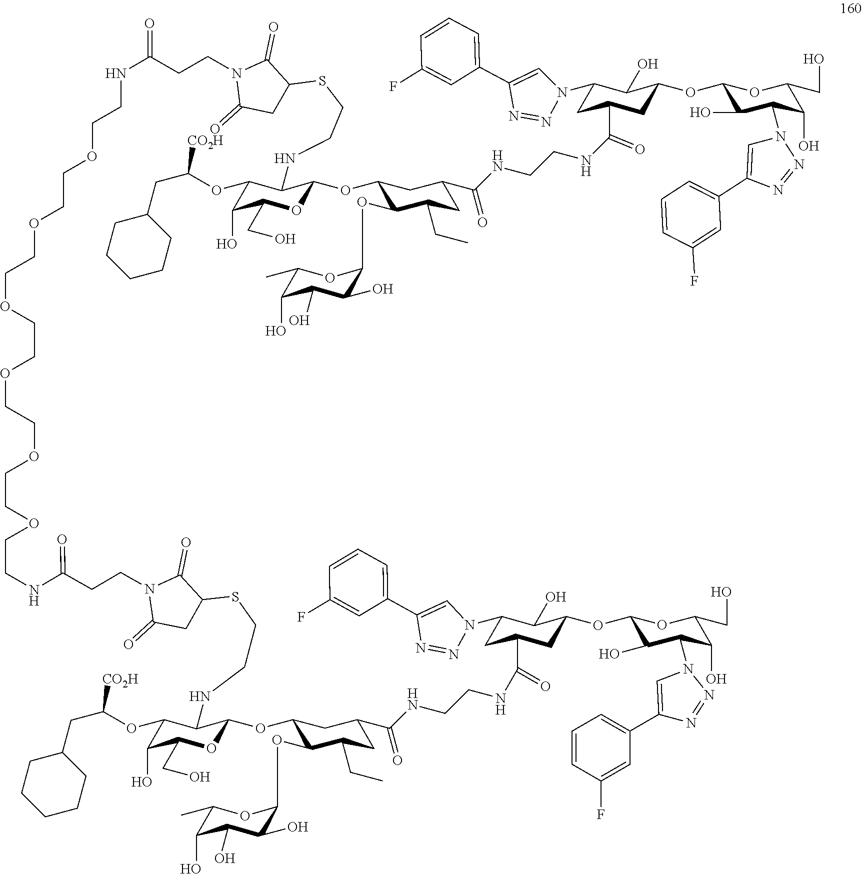

### Example 126

### Prophetic Synthesis of Multimeric Compound 161

Compound 161: Compound 161 can be prepared in an analogous fashion to FIG. 14 by replacing compound 22 with compound 39.

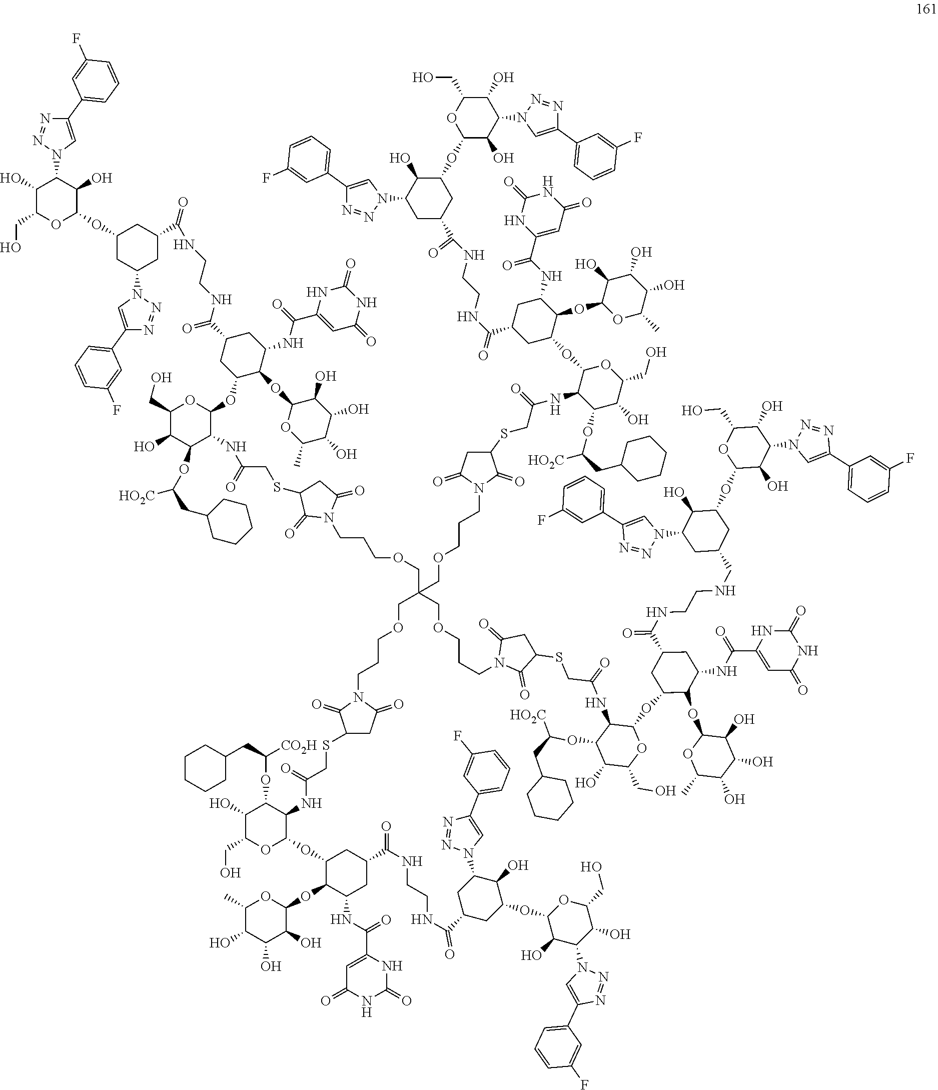

### Example 127

### Prophetic Synthesis of Multimeric Compound 162

Compound 162: Compound 162 can be prepared in an analogous fashion to FIG. 14 by replacing compound 22 with compound 40.

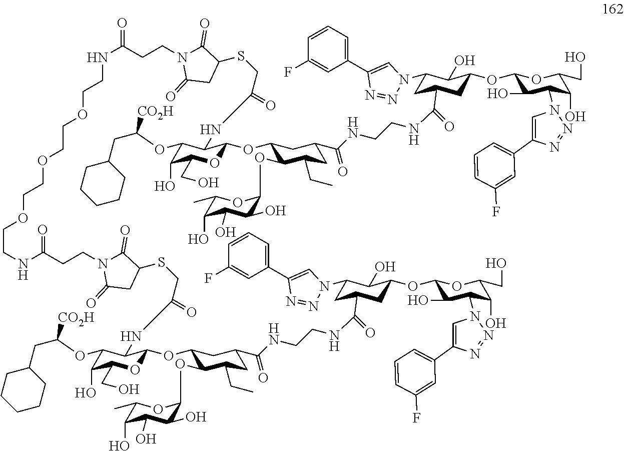

### Example 128

### Prophetic Synthesis of Multimeric Compound 163

Compound 163: Compound 163 can be prepared in an analogous fashion to FIG. 14 by replacing compound 22 with compound 46.

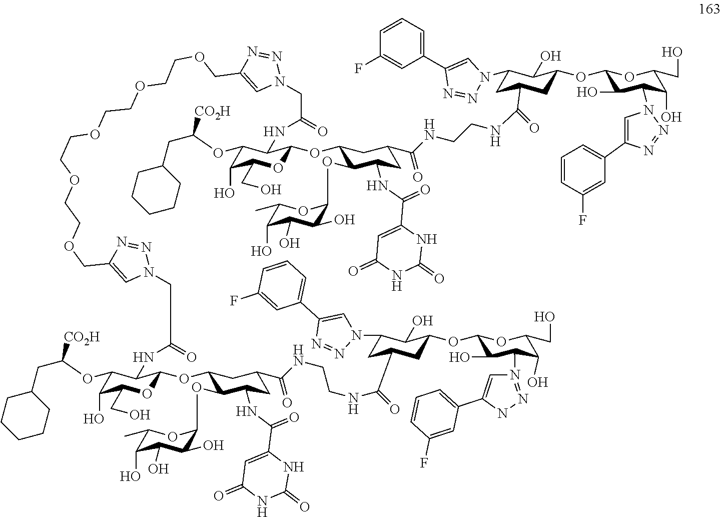

### Example 129

### Prophetic Synthesis of Multimeric Compound 164

Compound 164: Compound 164 can be prepared in an analogous fashion to FIG. 14 by replacing compound 22 with compound 47.

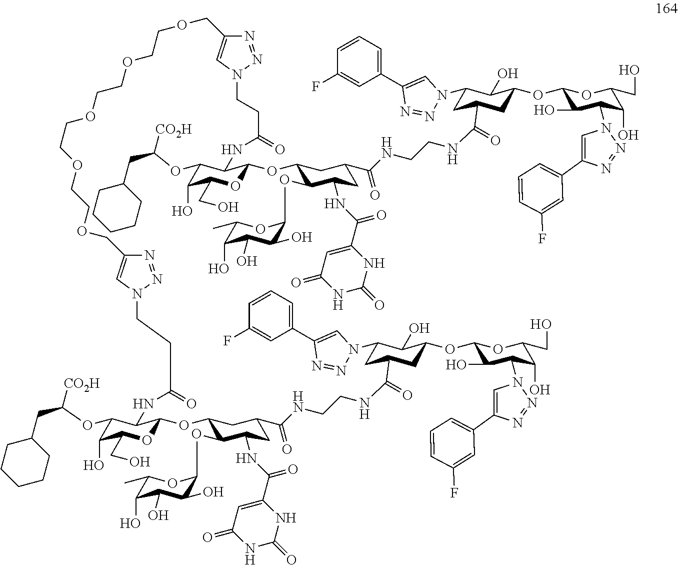

### Example 130

### Prophetic Synthesis of Multimeric Compound 165

Compound 165: Compound 165 can be prepared in an analogous fashion to FIG. 13 by replacing compound 22 with compound 48.

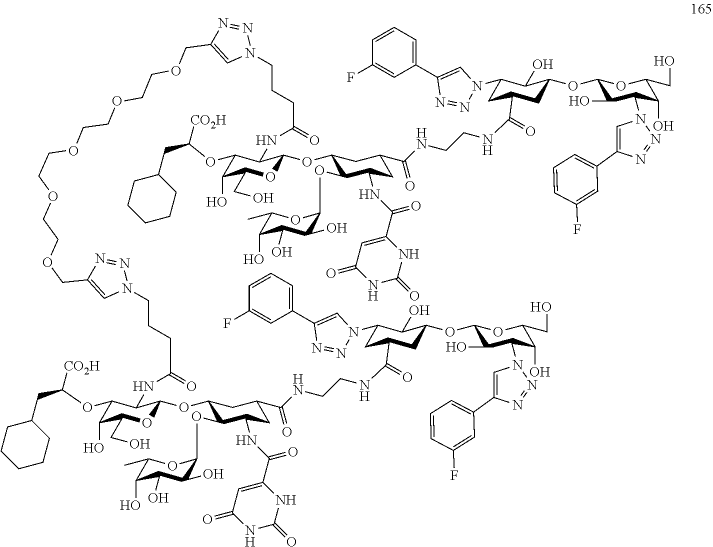

### Example 131

### Prophetic Synthesis of Multimeric Compound 166

Compound 166: Compound 166 can be prepared in an analogous fashion to FIG. 14 by replacing compound 22 with compound 49.

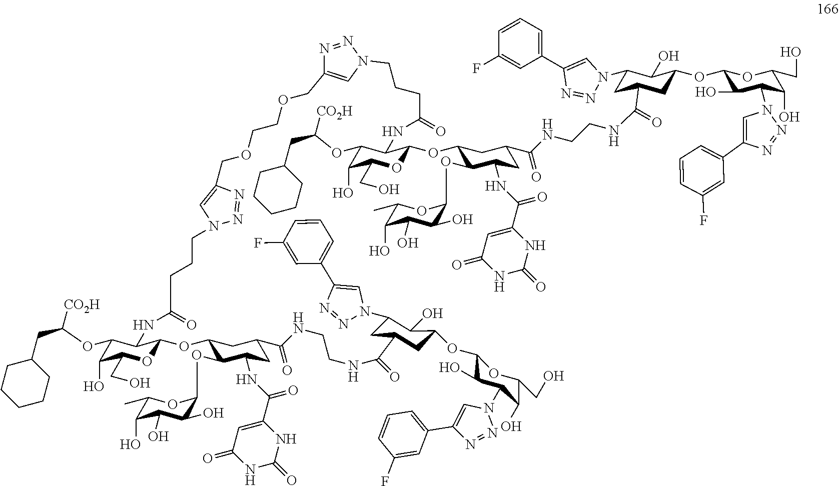

### Prophetic Synthesis of Multimeric Compound 167

Compound 167: Compound 167 can be prepared in an analogous fashion to FIG. 14 by replacing compound 22 with compound 50.

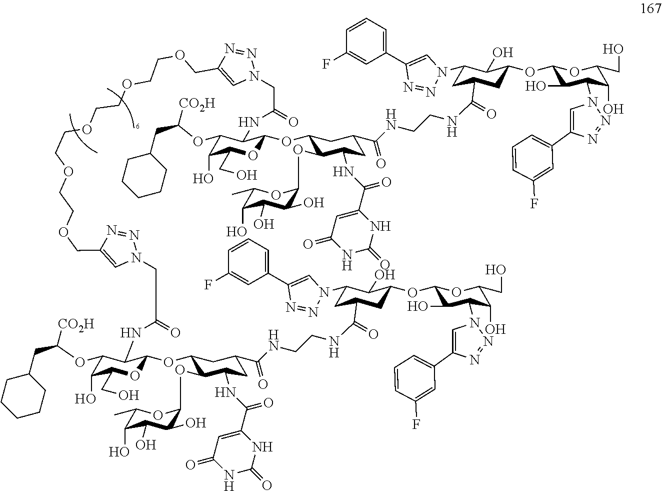

### Example 133

### Prophetic Synthesis of Multimeric Compound 168

Compound 168: Compound 168 can be prepared in an analogous fashion to FIG. 14 by replacing compound 22 with compound 51.

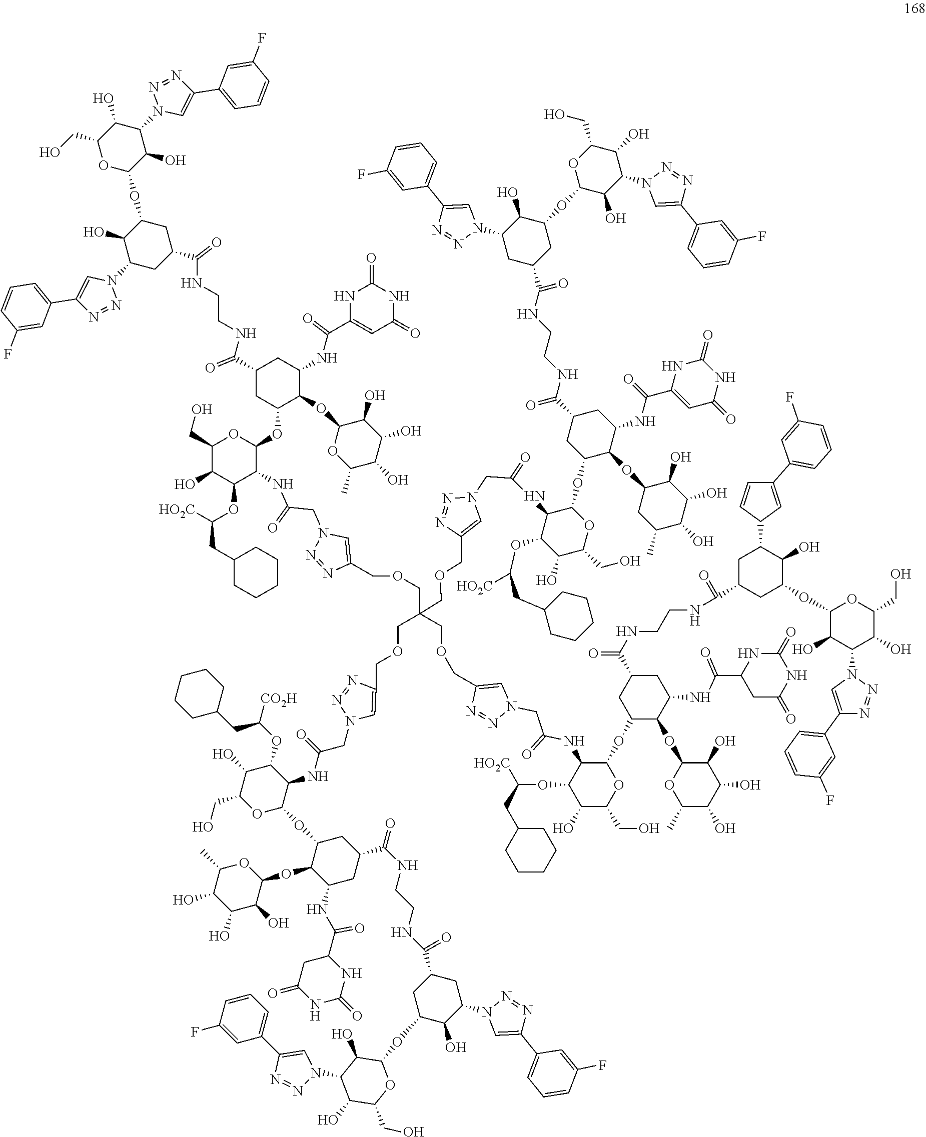

### Example 134

Compound 169: Compound 169 can be prepared in an analogous fashion to FIG. 14 by replacing compound 22 with compound 52.

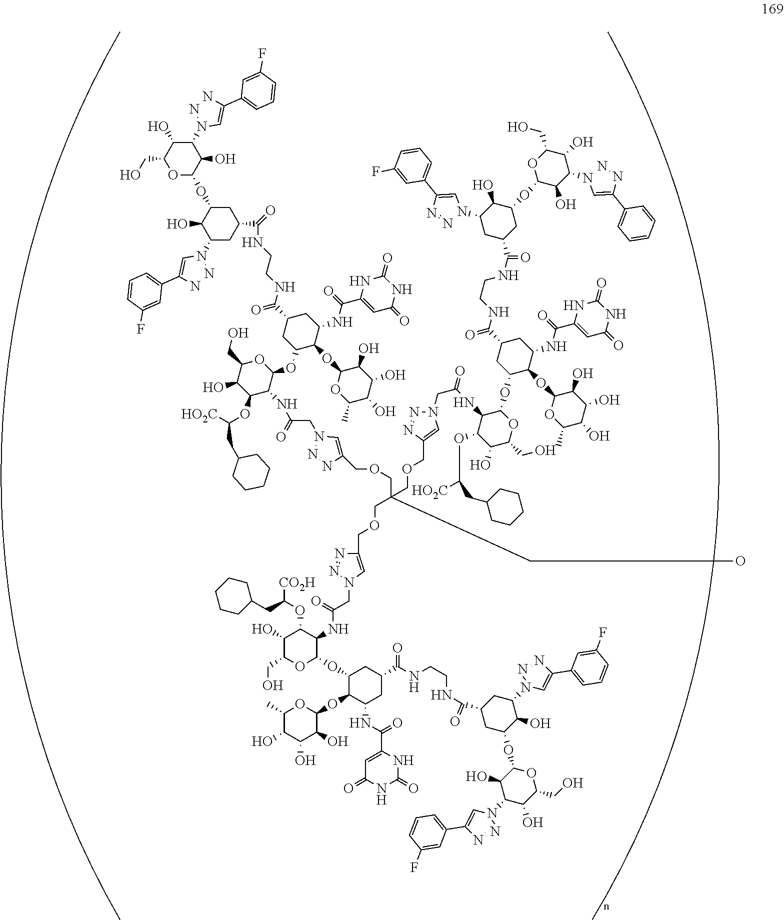

### Example 135

### Prophetic Synthesis of Multimeric Compound 170

Compound 170: Compound 170 can be prepared in an analogous fashion to FIG. 14 by replacing compound 22 with compound 53.

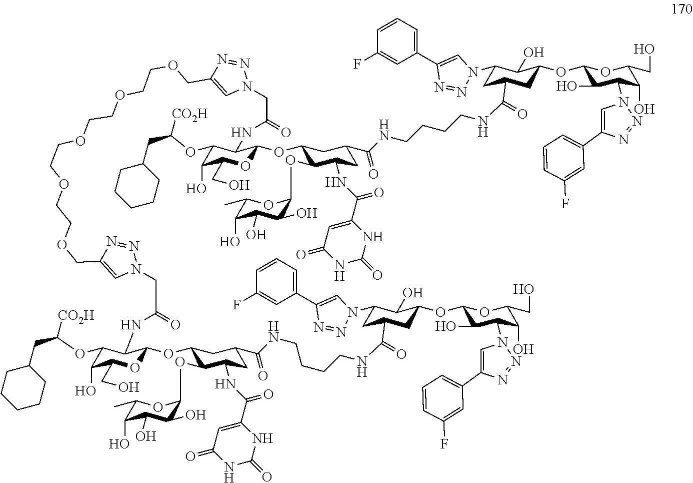

### Example 136

### Prophetic Synthesis of Multimeric Compound 171

Compound 171: Compound 171 can be prepared in an analogous fashion to FIG. 14 by replacing compound 22 with compound 54.

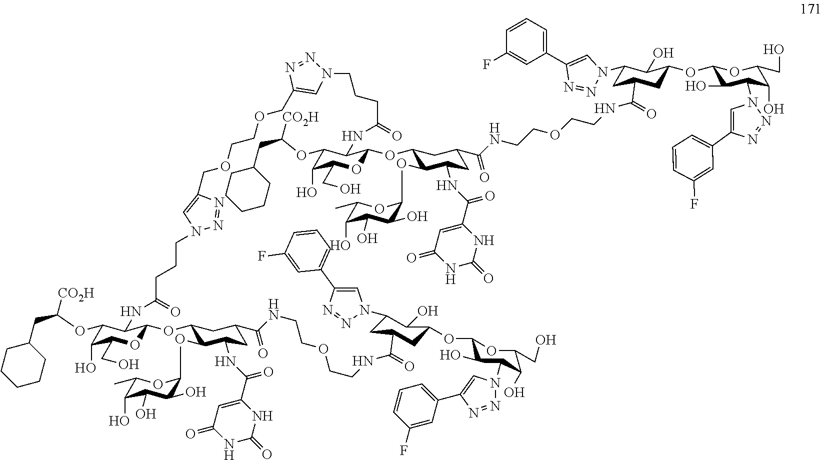

### Example 137

### Prophetic Synthesis of Multimeric Compound 172

Compound 172: Compound 172 can be prepared in an analogous fashion to FIG. 14 by replacing compound 22 with compound 56.

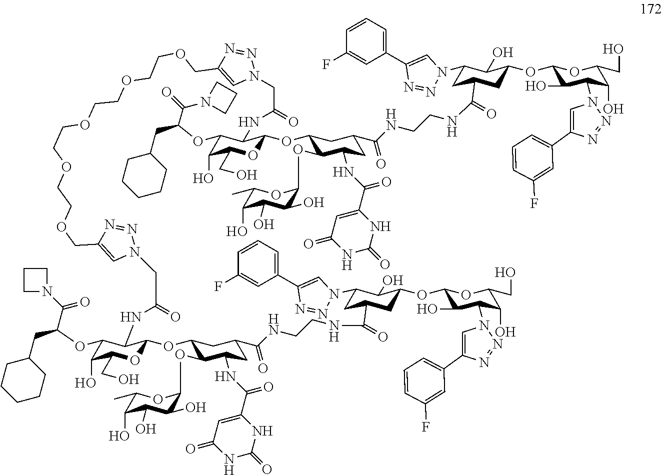

### Example 138

### Prophetic Synthesis of Multimeric Compound 173

Compound 173: Compound 173 can be prepared in an analogous fashion to FIG. 14 by replacing compound 22 with compound 57.

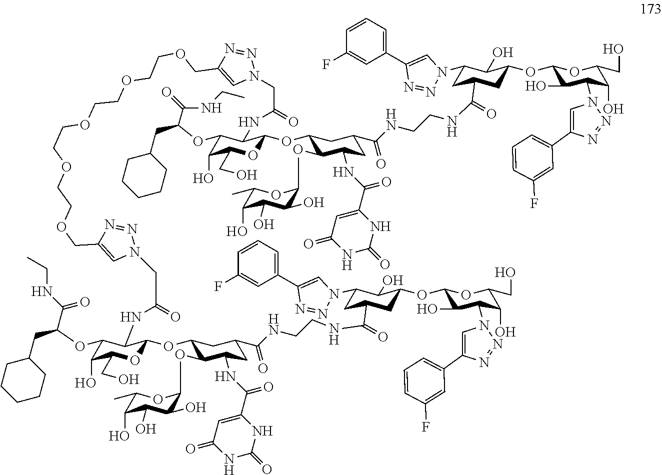

### Example 139

### Prophetic Synthesis of Multimeric Compound 174

Compound 174: Compound 174 can be prepared in an analogous fashion to FIG. 14 by replacing compound 22 with compound 58.

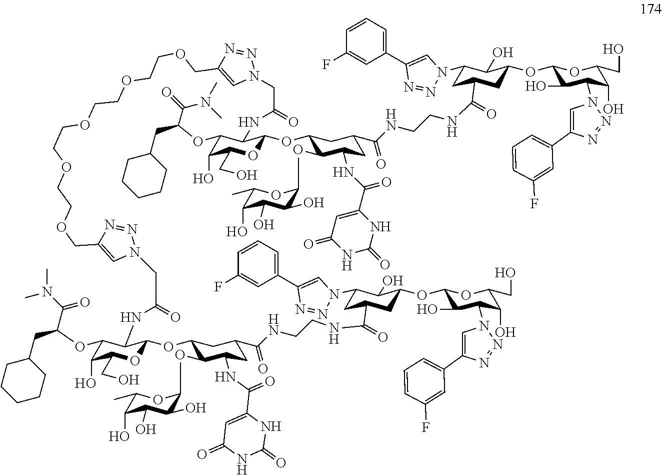

### Example 140

### Prophetic Synthesis of Multimeric Compound 175

Compound 175: Compound 175 can be prepared in an analogous fashion to FIG. 14 by replacing compound 22 with compound 59.

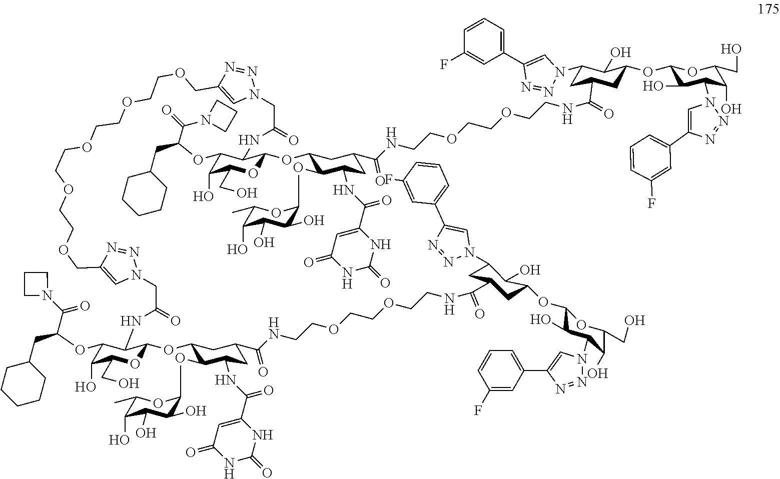

### Example 141

### Prophetic Synthesis of Multimeric Compound 176

Compound 176: Compound 176 can be prepared in an analogous fashion to FIG. 14 by replacing compound 22 with compound 68.

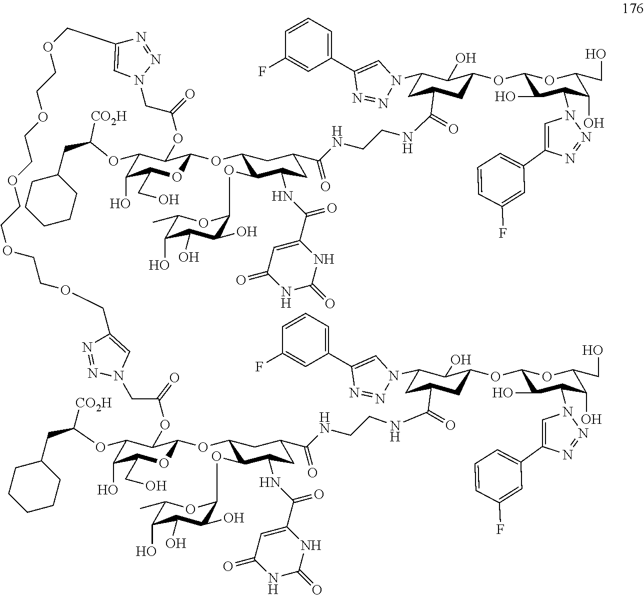

### Example 142

### Prophetic Synthesis of Multimeric Compound 177

Compound 177: Compound 177 can be prepared in an analogous fashion to FIG. 14 by replacing compound 22 with compound 69.

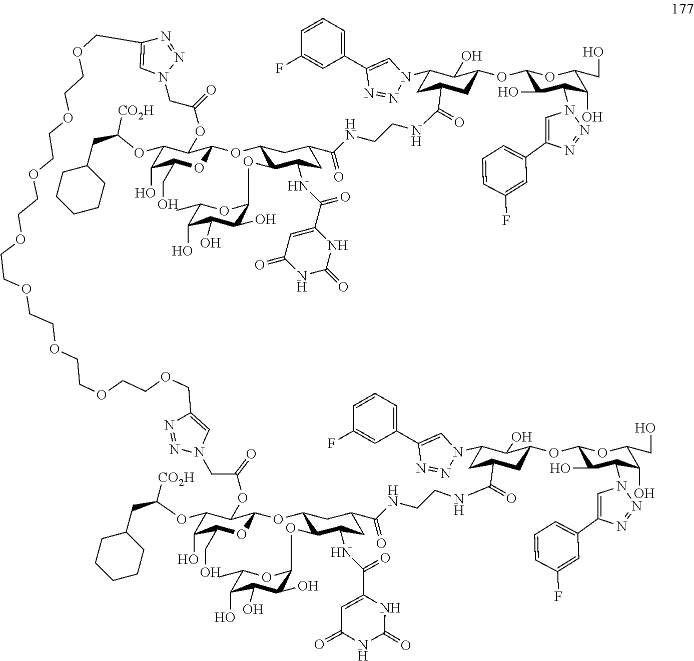

### Example 143

### Prophetic Synthesis of Multimeric Compound 178

Compound 178: Compound 178 can be prepared in an analogous fashion to FIG. 14 by replacing compound 22 with compound 70.

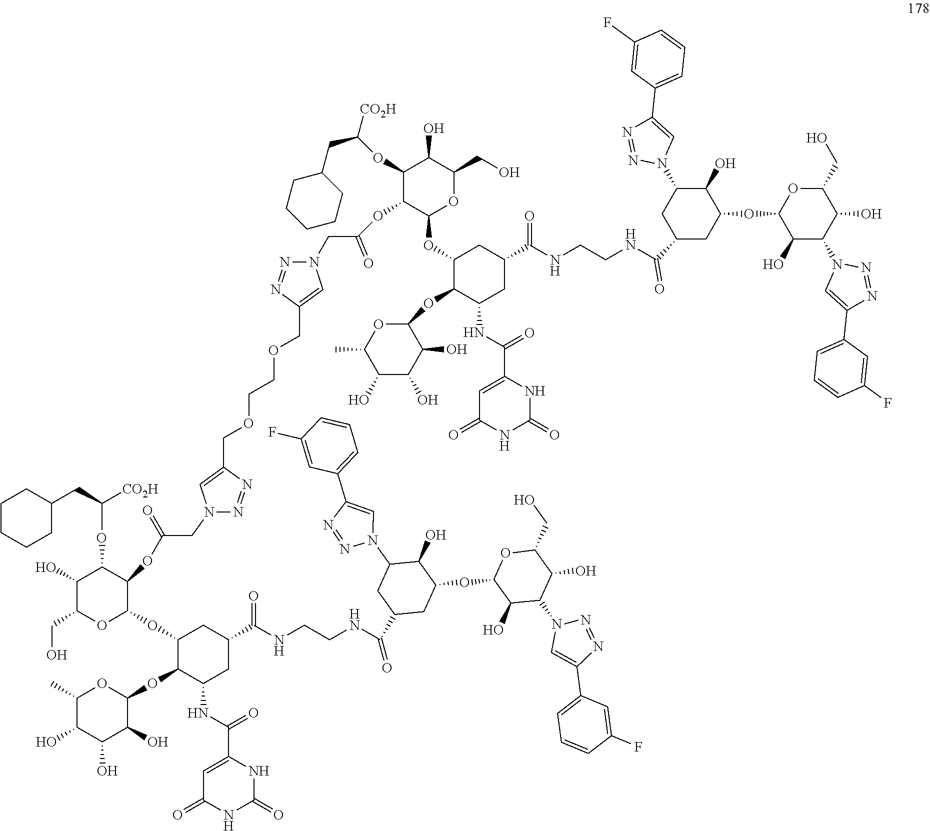

### Example 144

### Prophetic Synthesis of Multimeric Compound 179

Compound 179: Compound 179 can be prepared in an analogous fashion to FIG. 14 by replacing compound 22 with compound 71.

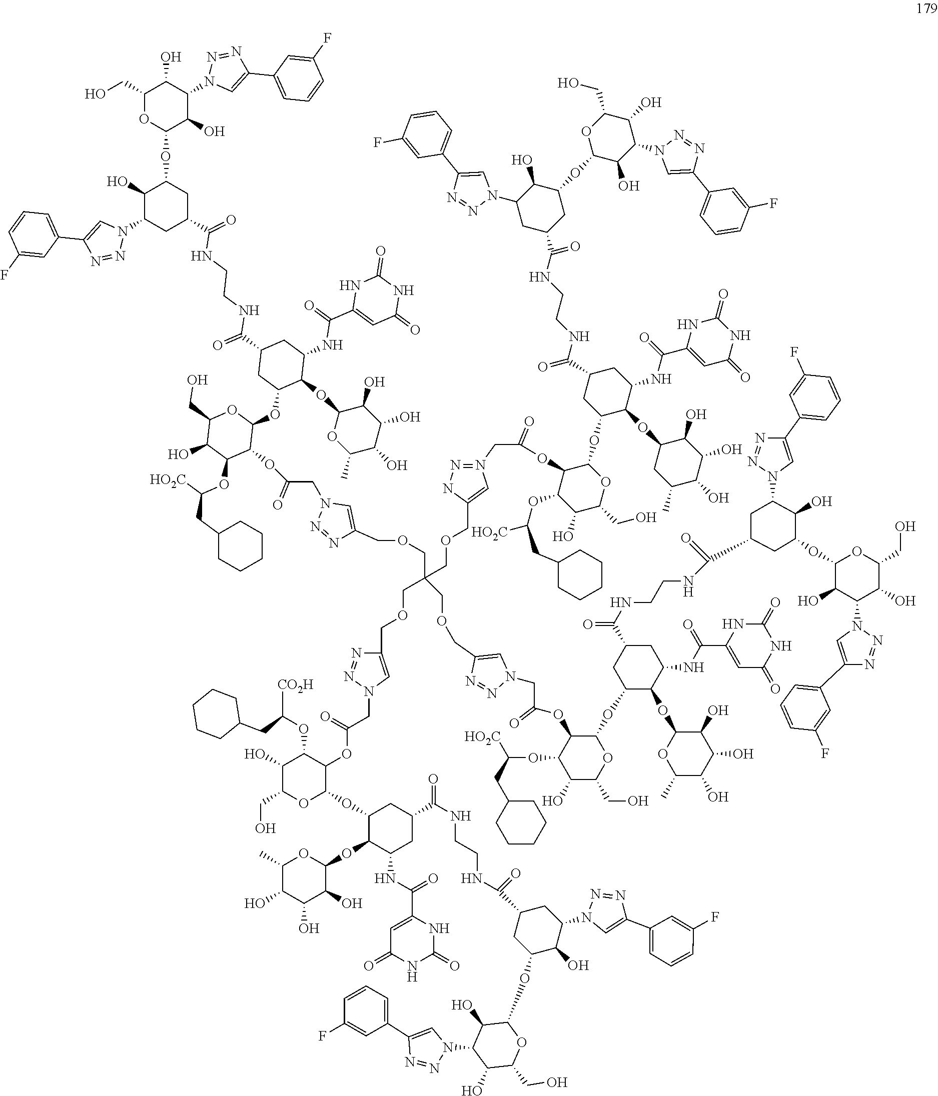

### Example 145

### Prophetic Synthesis of Multimeric Compound 180

Compound 180: Compound 180 can be prepared in an analogous fashion to FIG. 14 by replacing compound 22 with compound 73.

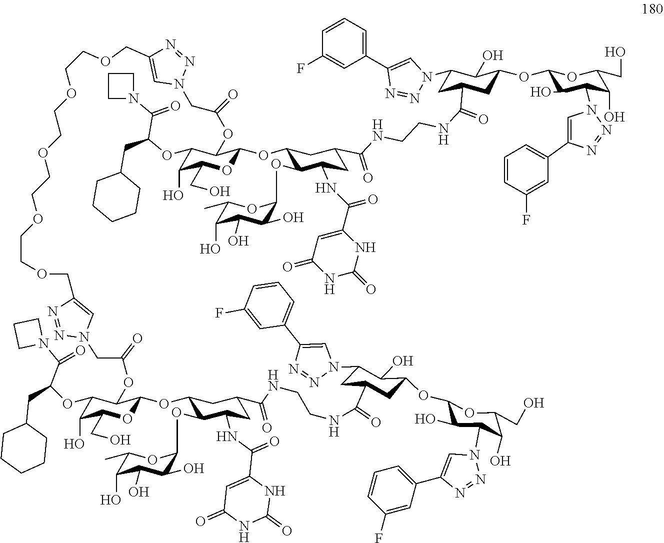

### Example 146

### Prophetic Synthesis of Multimeric Compound 181

Compound 181: Compound 181 can be prepared in an analogous fashion to FIG. 14 by replacing compound 22 with compound 78.

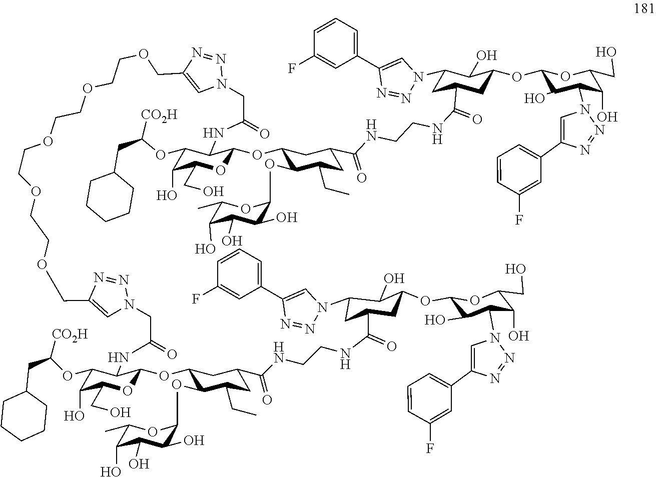

### Example 147

### Prophetic Synthesis of Multimeric Compound 182

Compound 182: Compound 182 can be prepared in an analogous fashion to FIG. 14 by replacing compound 22 with compound 79.

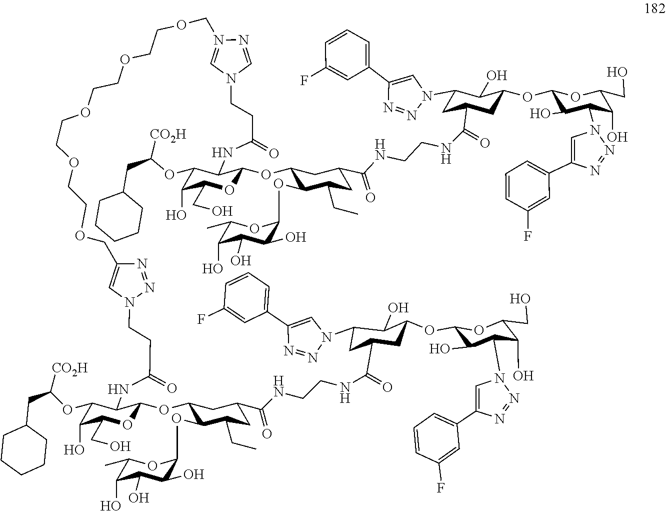

### Example 148

### Prophetic Synthesis of Multimeric Compound 183

Compound 183: Compound 183 can be prepared in an analogous fashion to FIG. 14 by replacing compound 22 with compound 80.

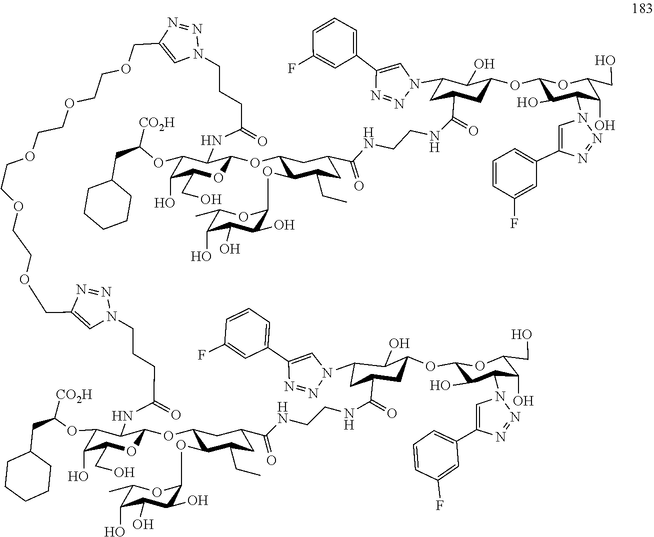

### Example 149

### Prophetic Synthesis of Multimeric Compound 184

Compound 184: Compound 184 can be prepared in an analogous fashion to FIG. 14 by replacing compound 22 with compound 81.

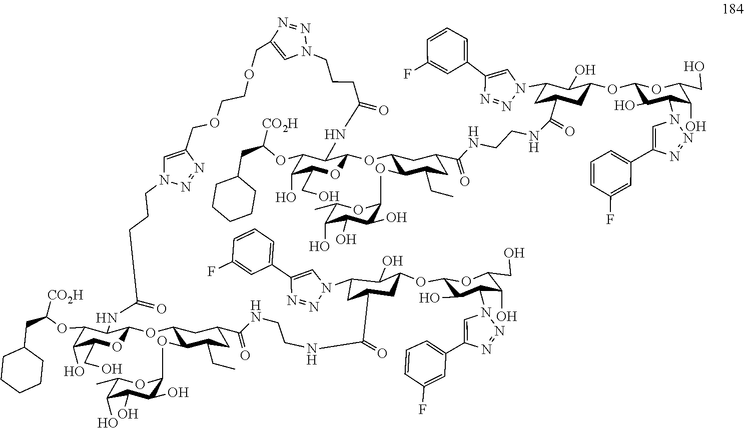

### Example 150

### Prophetic Synthesis of Multimeric Compound 185

Compound 185: Compound 185 can be prepared in an analogous fashion to FIG. 14 by replacing compound 22 with compound 82.

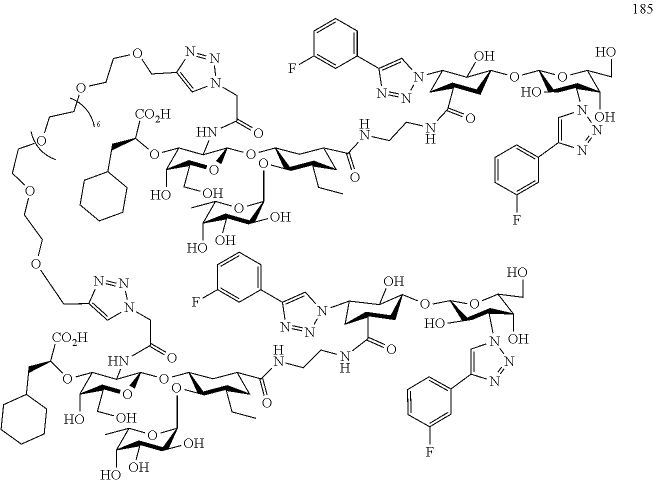

### Example 151

### Prophetic Synthesis of Multimeric Compound 186

Compound 186: Compound 186 can be prepared in an analogous fashion to FIG. 14 by replacing compound 22 with compound 83.

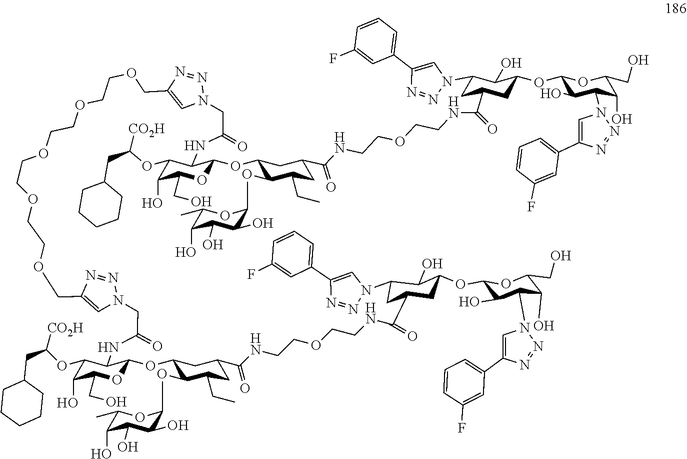

### Example 152

### Prophetic Synthesis of Multimeric Compound 187

Compound 187: Compound 187 can be prepared in an analogous fashion to FIG. 14 by replacing compound 22 with compound 84.

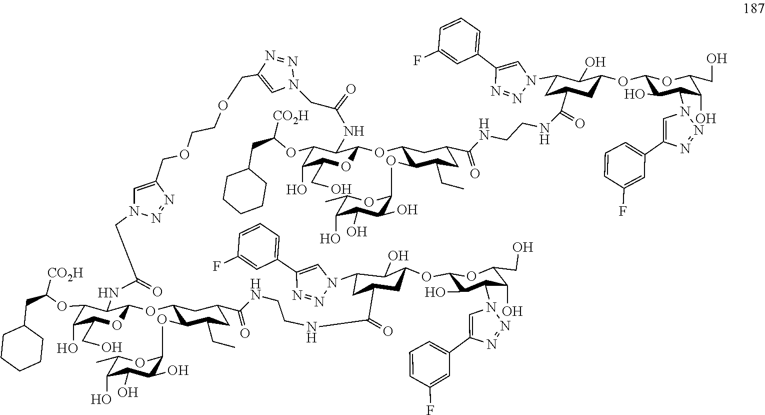

### Example 153

### Prophetic Synthesis of Multimeric Compound 188

Compound 188: Compound 188 can be prepared in an analogous fashion to FIG. 14 by replacing compound 22 with compound 85.

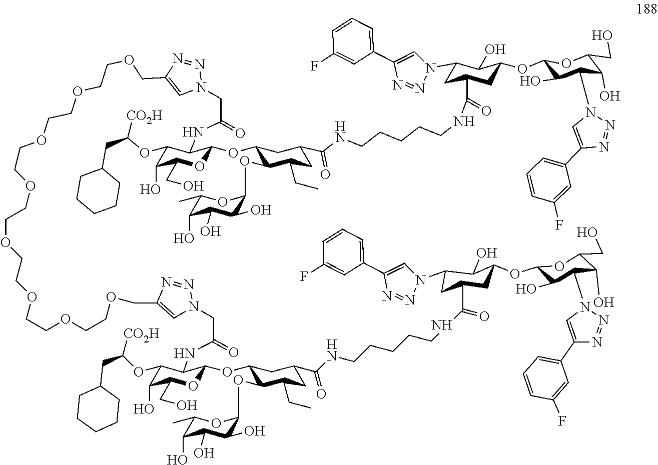

### Example 154

### Prophetic Synthesis of Multimeric Compound 189

Compound 189: Compound 189 can be prepared in an analogous fashion to FIG. 14 by replacing compound 22 with compound 87.

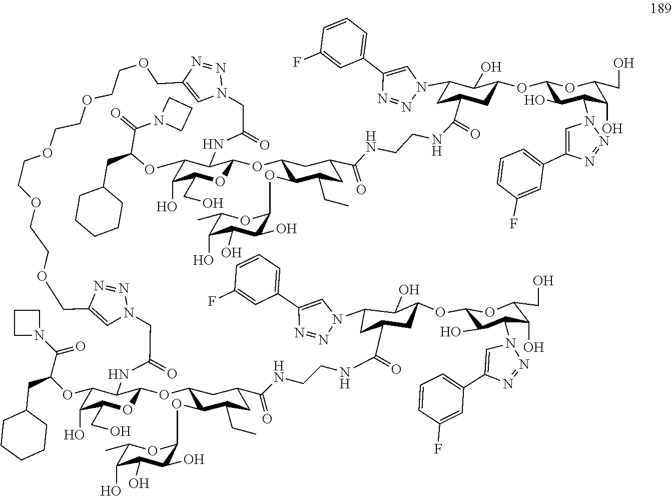

### Example 155

Compound 190: Compound 190 can be prepared in an analogous fashion to FIG. 14 by replacing compound 22 with compound 88.

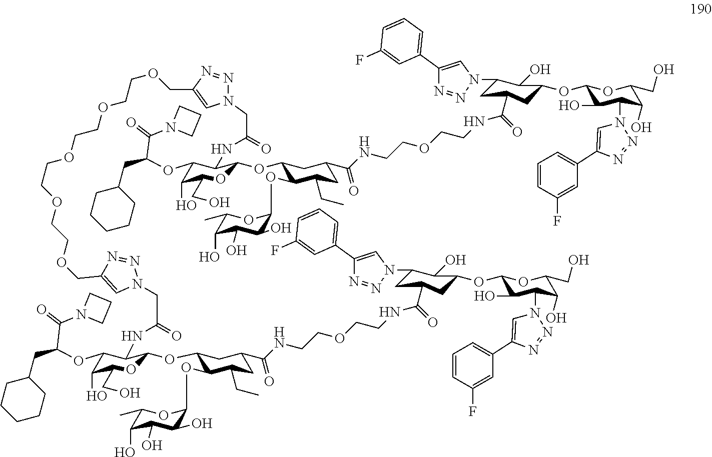

### Example 156

### Prophetic Synthesis of Multimeric Compound 191

Compound 191: Compound 191 can be prepared in an analogous fashion to FIG. 14 by replacing compound 22 with compound 89.

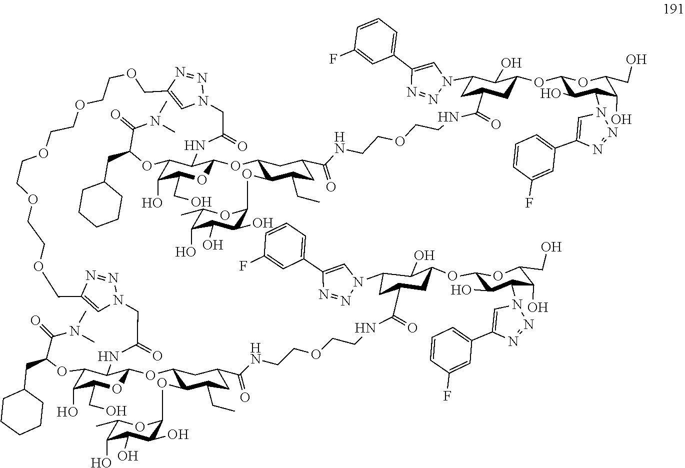

### Example 157

### Prophetic Synthesis of Multimeric Compound 192

Compound 192: Compound 192 can be prepared in an analogous fashion to FIG. 14 by replacing compound 22 with compound 90.

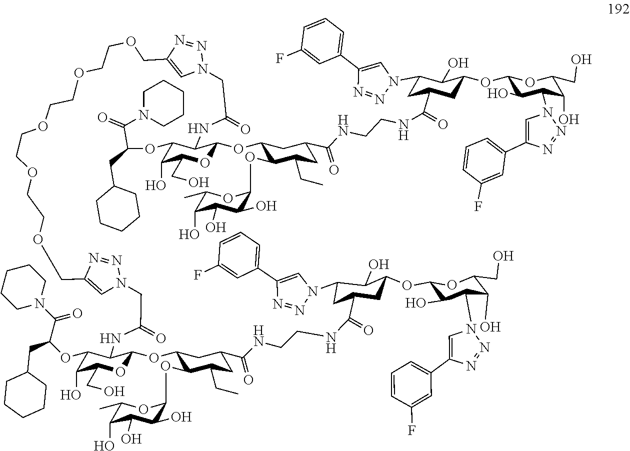

### Example 158

### Prophetic Synthesis of Multimeric Compound 193

Compound 193: Compound 193 can be prepared in an analogous fashion to FIG. 14 by replacing compound 22 with compound 91.

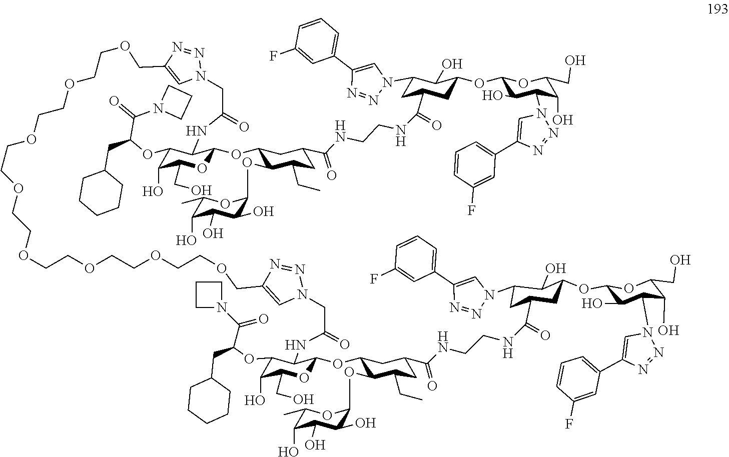

### Example 159

### Prophetic Synthesis of Multimeric Compound 194

Compound 194: Compound 194 can be prepared in an analogous fashion to FIG. 14 by replacing compound 22 with compound 92.

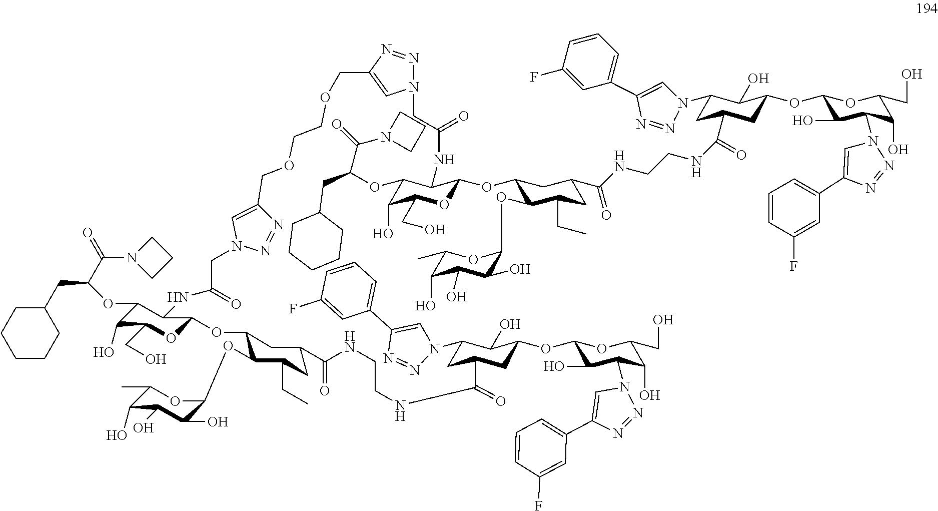

### Example 160

### Prophetic Synthesis of Multimeric Compound 195

Compound 195: Compound 195 can be prepared in an analogous fashion to FIG. 14 by replacing compound 22 with compound 93.

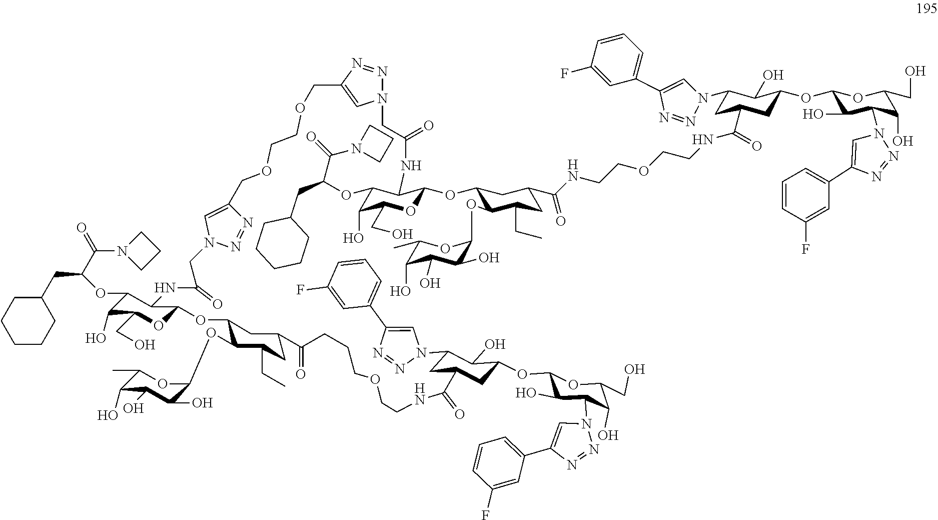

### Example 161

### Prophetic Synthesis of Multimeric Compound 197

Compound 197: To a solution of compound 22 (1 eq) in anhydrous DMSO was acetic acid NHS ester (compound 196) (5 eq). The mixture was stirred at ambient temperature for 12 hours. The solvent was removed in vacuo and the residue was purified by HPLC to afford compound 197.

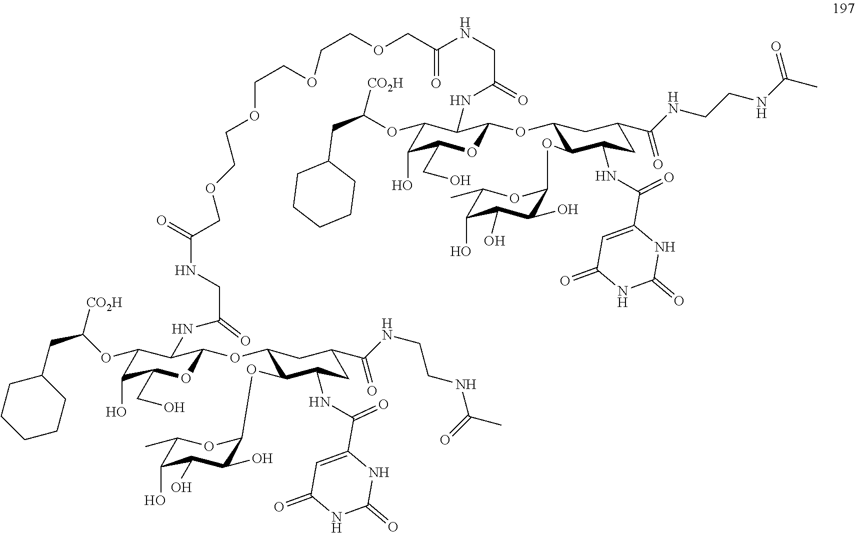

### Example 162

### Prophetic Synthesis of Multimeric Compound 198

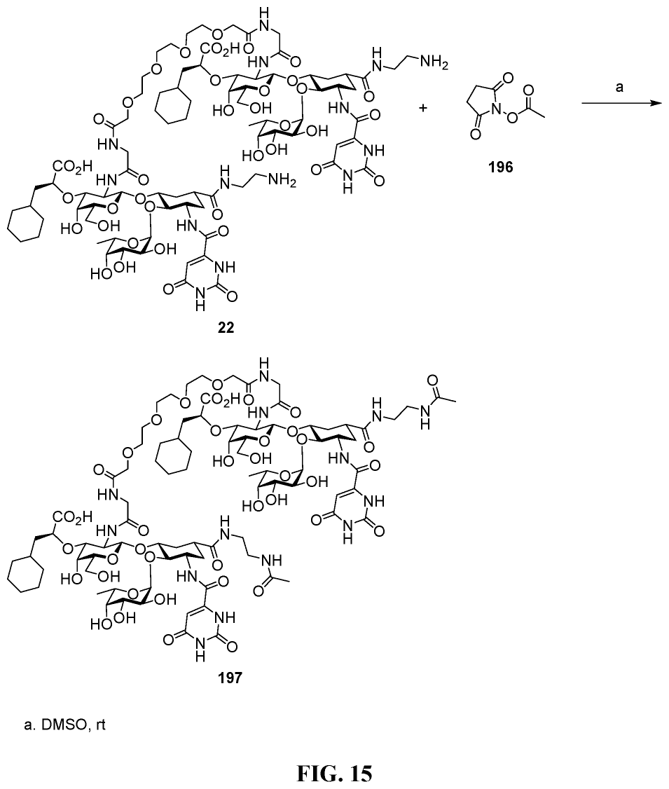

Compound 198: Compound 198 can be prepared in an analogous fashion to FIG. 15 by replacing compound 196 with NHS-methoxyacetate.

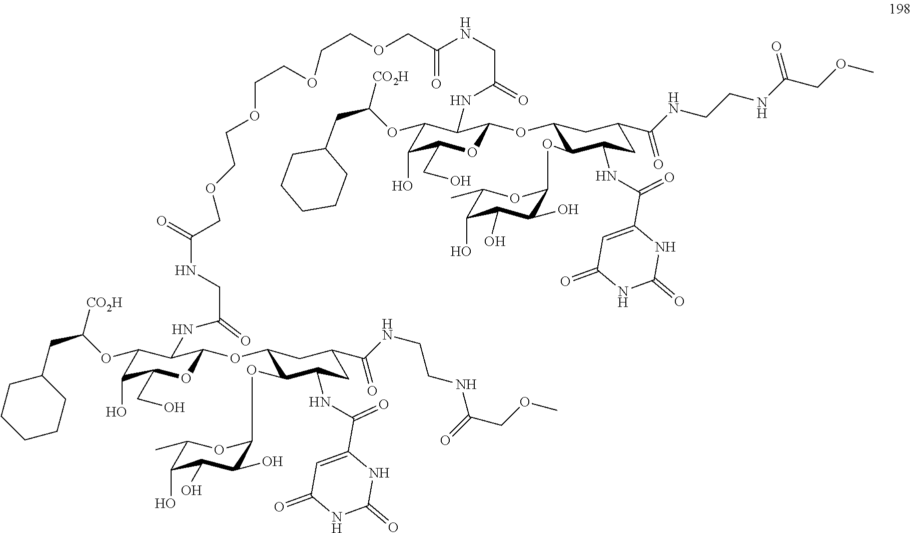

### Example 163

### Prophetic Synthesis of Multimeric Compound 199

Compound 199: Compound 199 can be prepared in an analogous fashion to FIG. 15 by replacing compound 196 with PEG-12 propionic acid NHS ester.

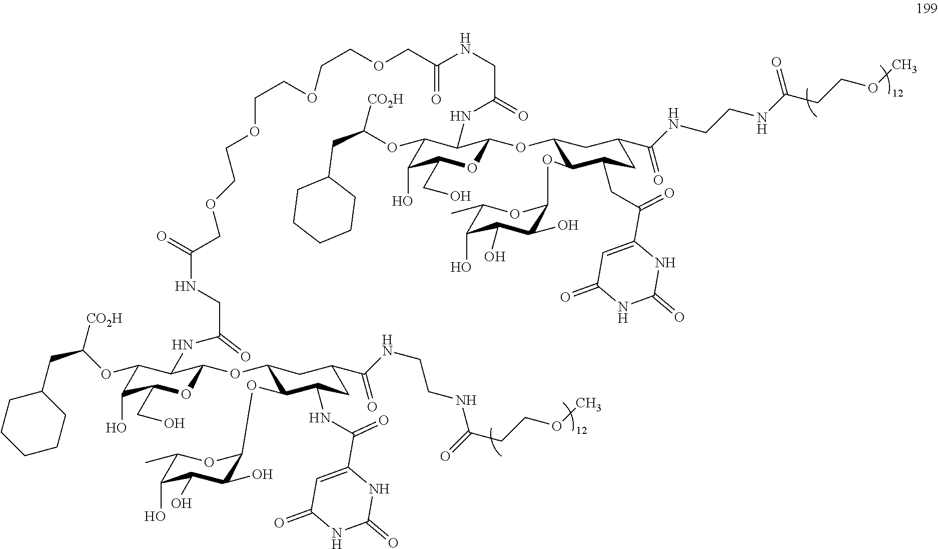

### Example 164

### Prophetic Synthesis of Multimeric Compound 200

Compound 200: Compound 200 can be prepared in an analogous fashion to FIG. 15 by replacing compound 22 with compound 78.

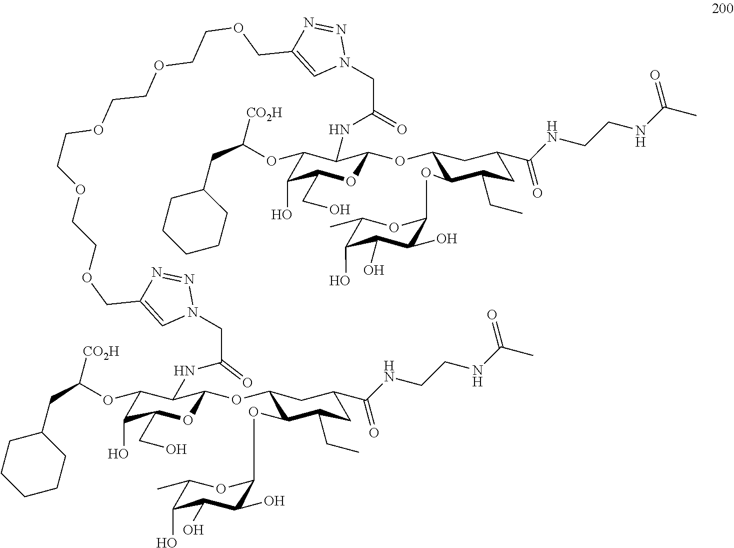

### Example 165

### Prophetic Synthesis of Multimeric Compound 201

Compound 201: Compound 201 can be prepared in an analogous fashion to FIG. 15 by replacing compound 22 with compound 78 and replacing compound 196 with NHS-methoxyacetate.

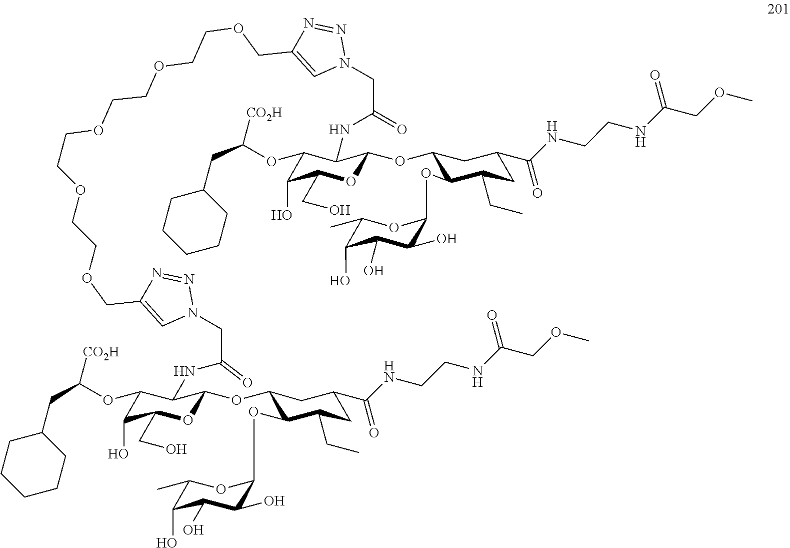

### Example 166

### Prophetic Synthesis of Multimeric Compound 202

Compound 202: Compound 202 can be prepared in an analogous fashion to FIG. 15 by replacing compound 22 with compound 78 and replacing compound 196 with PEG-12 propionic acid NHS ester.

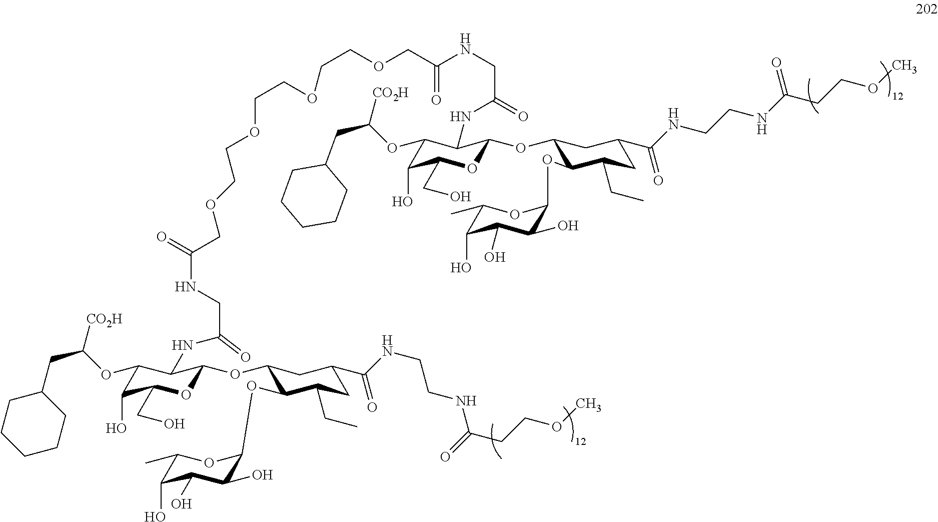

### Prophetic Synthesis of Multimeric Compound 203

Compound 203: Compound 203 can be prepared in an analogous fashion to FIG. 15 by replacing compound 22 with compound 78.

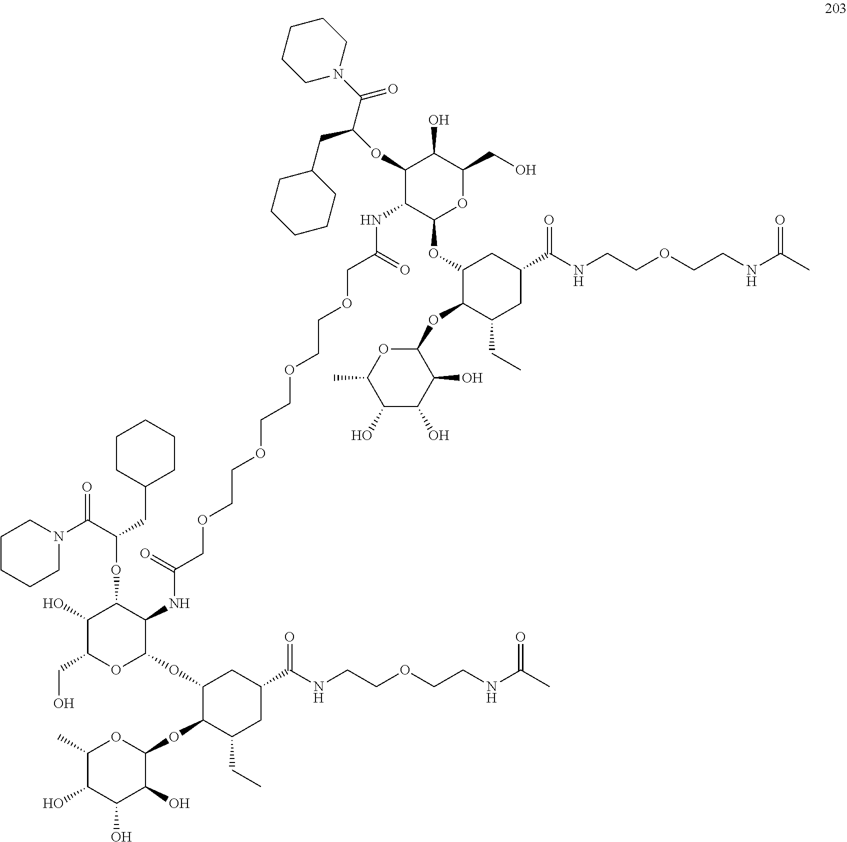

### Example 167

### Synthesis of Multimeric Compound 206

Compound 205: A solution of compound 204 (synthesis described in Mead, G. et. al., Bioconj. Chem., 2015, 25, 1444-1452) (0.25 g, 0.53 mmole) and propiolic acid (0.33 mL, 5.30 mmole, 10 eq) in distilled water (1.5 mL) was degassed. A solution of CuSO₄/THPTA in distilled water (0.04 M) (1.3 mL, 53 μmole, 0.1 eq) and sodium ascorbate (21 mg, 0.11 mmole, 0.2 eq) were added successively and the resulting solution was stirred 3 hrs at room temperature. The reaction mixture was concentrated under reduced pressure and partially purified by C-18 column chromatography (water/MeOH, water only—5/5, v/v). The resulting material was further purified by C-18 column chromatography eluting with water to afford compound 205 (0.16 g, 0.34 mmole, 64%). MS: (Calculated for C₈H₁₀₃N₃Na₃O₁₄S₃, 537.34), ES-Negative (513.5, M−Na−1).

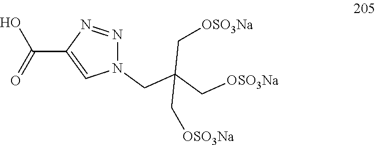

Compound 206: To a solution of compound 205 (7.5 mg, 14 μmole), DIPEA (2.4 μL, 14 μmole) and a catalytic amount of DMAP in DMF/DMSO (3/1, v/v, 0.15 mL) at 0° C. was added EDCI (1.6 mg, 8.22 μmole). The solution was stirred for 20 min. This solution was slowly added to a solution of compound 78 (5.0 mg, 2.7 μmole) in DMF/DMSO (3/1, v/v, 0.2 mL) cooled at 0° C. The resulting solution was stirred 12 hrs allowing the reaction temperature to increase to room temperature. The reaction mixture was purified directly by HPLC. The product portions were collected, concentrated under reduced pressure, then lyophilized to give compound 206 as a white solid (0.4 mg, 1.15 μmole, 1.1%). MS: Calculated (C₉₈H₁₅₄N₁₈Na₆O₅₉S₆, 2856.7), ES-Negative (907.7, M/3; 881.0, M-ISO₃/3; 854.1 M−2SO₃/3; 685.8 M+1Na/4; 680.5 M/4), Fraction of RT=10.65 min, 1399.4, M+7Na-1SO₃/2; 959.3 M+7Na/3; M+7Na−ISO3/3; 724.8, M+8Na/4; 549.M+1Na/5; 460.9 M+2Na/6; 401.M+4Na/7).

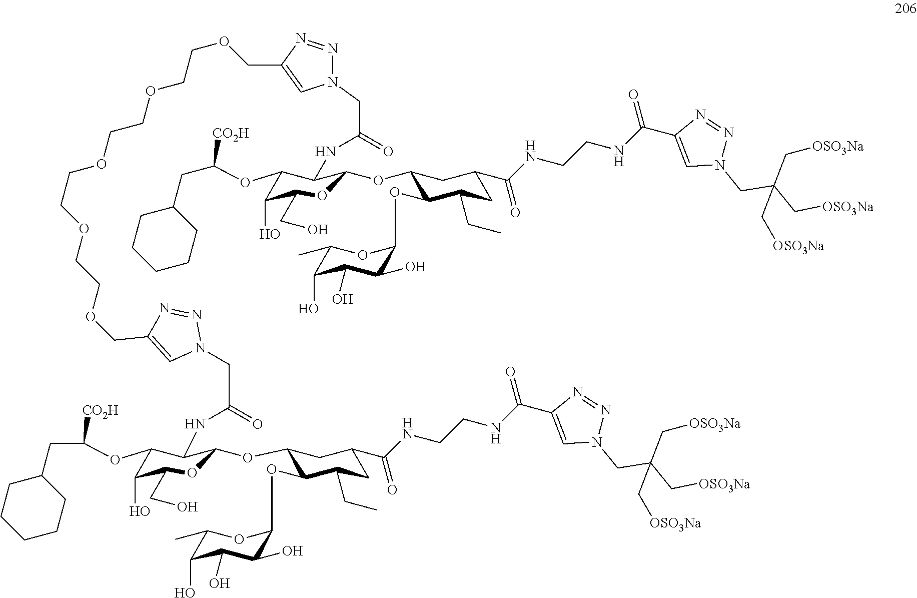

### Example 168

### Prophetic Synthesis of Multimeric Compound 207

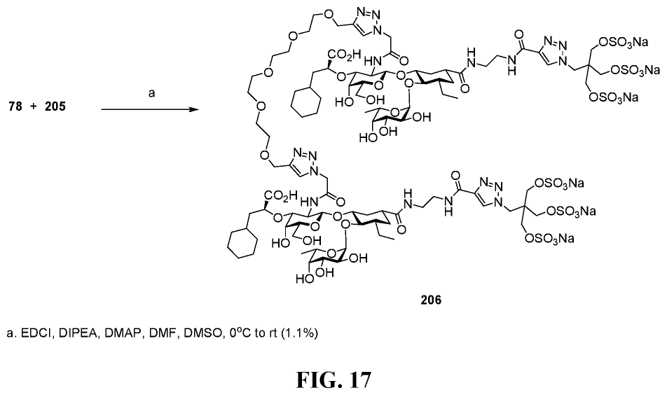

Compound 207: Compound 207 can be prepared in an analogous fashion to FIG. 17 by replacing compound 78 with compound 22.

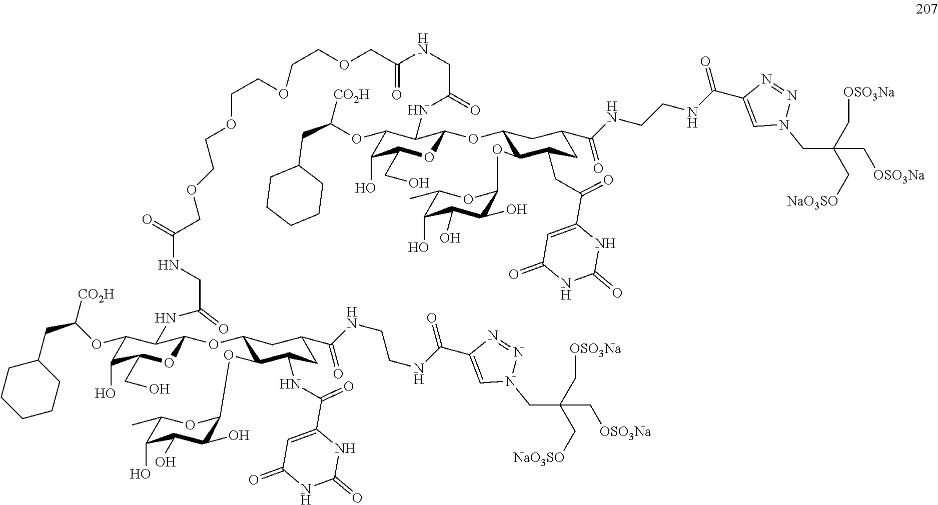

### Example 169

### Prophetic Synthesis of Multimeric Compound 208

Compound 208: Compound 208 can be prepared in an analogous fashion to FIG. 14 by replacing compound 83 with compound 78.

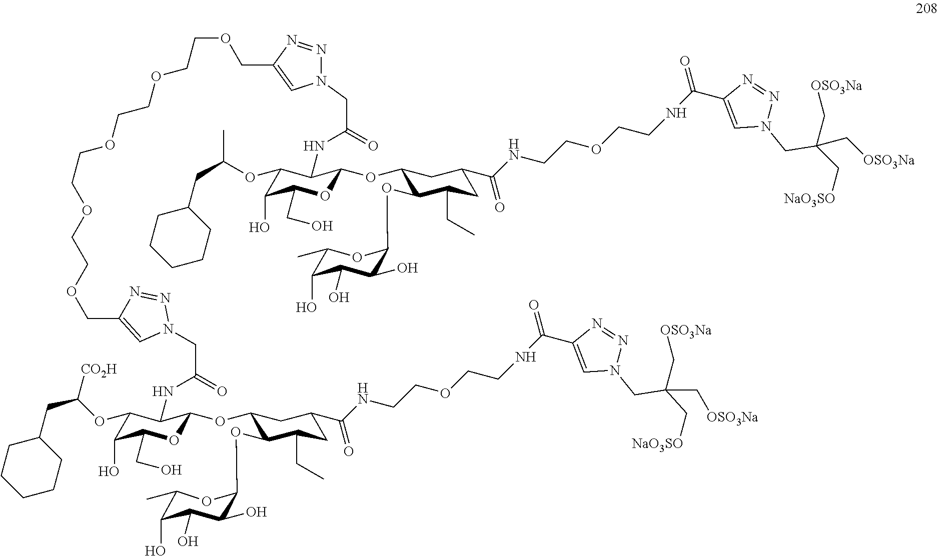

### Example 170

### Prophetic Synthesis of Multimeric Compound 209

Compound 209: Compound 209 can be prepared in an analogous fashion to FIG. 17 using compound 87 in place of compound 78.

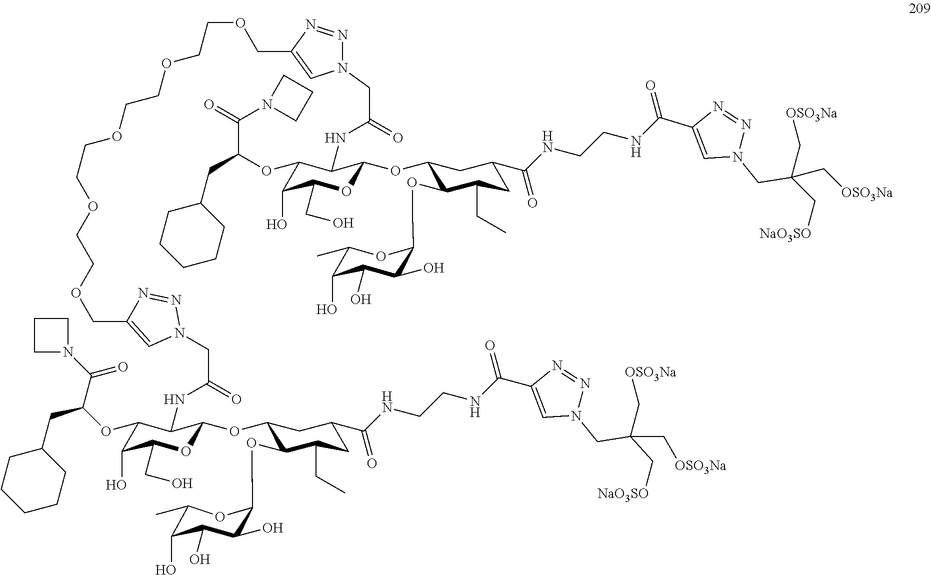

### Example 171

### Prophetic Synthesis of Multimeric Compound 210

Compound 210: Compound 210 can be prepared in an analogous fashion to FIG. 17 using compound 93 in place of compound 78.

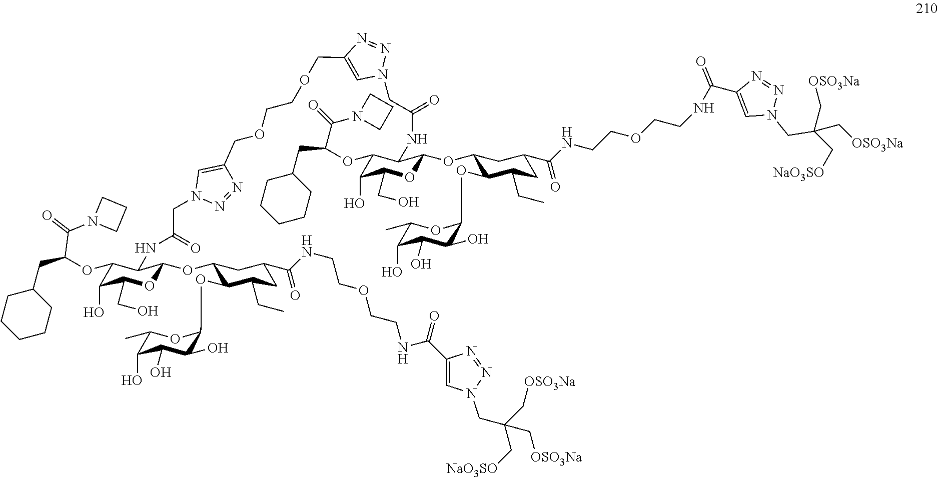

### Example 172

### Prophetic Synthesis of Multimeric Compound 211

Compound 211: Compound 211 can be prepared in an analogous fashion to FIG. 17 using compound 37 in place of compound 78.

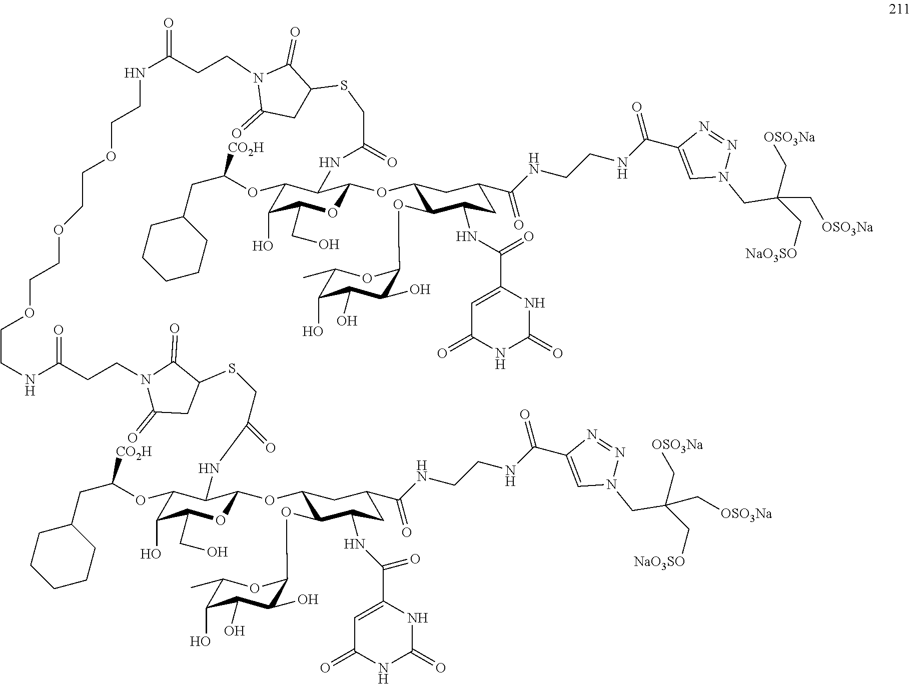

### Example 173

### Synthesis of Multimeric Compound 218

Compound 213: Prepared according to Bioorg. Med. Chem. Lett. 1995, 5, 2321-2324 starting with D-threonolactone.

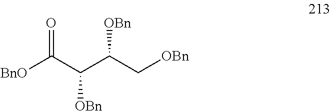 [SMILES: O=C(OCc1ccccc1)[C@@H](OCc1ccccc1)[C@@H](COCc1ccccc1)OCc1ccccc1]

Compound 214: Compound 213 (500 mg, 1 mmol) was dissolved in 9 mL acetonitrile. Potassium hydroxide (1 mL of a 2M solution) was added and the reaction mixture was stirred at 50° C. for 12 hours. The reaction mixture was partitioned between dichloromethane and water. The phases were separated and the aqueous phase was extracted 3 times with dichloromethane. The aqueous phase was acidified with 1N HCl until pH˜1 and extracted 3 times with dichloromethane. The combined dichloromethane extracts from after acidification of the aqueous phase were concentrated in vacuo to give compound 214 as a yellow oil (406 mg). LCMS (C-18; 5-95 H₂O/MeCN): UV (peak at 4.973 min), positive mode: m/z=407 [M+H]⁺; negative mode: m/z=405 [M−H]⁻C₂₅H₂₆O⁵ (406).

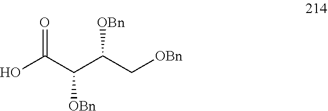 [SMILES: O=C(O)[C@@H](OCc1ccccc1)[C@@H](COCc1ccccc1)OCc1ccccc1]

Compound 215: Prepared in an analogous fashion to compound 214 using L-erythronolactone as the starting material. LCMS (C-18; 5-95 H₂O/MeCN): ELSD (5.08 min), UV (peak at 4.958 min), positive mode: m/z=407 [M+H]⁺; negative mode: m/z=405 [M−H]⁻C₂₅H₂₆O₅ (406).

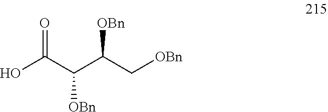 [SMILES: O=C(O)[C@@H](OCc1ccccc1)[C@H](COCc1ccccc1)OCc1ccccc1]

Compound 216: Prepared in an analogous fashion to compound 214 using L-threonolactone as the starting material. LCMS (C-18; 5-95 H₂O/MeCN): ELSD (5.08 min), UV (peak at 4.958 min), positive mode: m/z=407 [M+H]⁺; negative mode: m/z=405 [M−H]⁻C₂₅H₂₆O₅ (406).

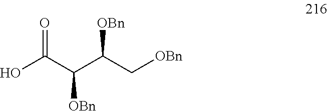 [SMILES: O=C(O)[C@H](OCc1ccccc1)[C@H](COCc1ccccc1)OCc1ccccc1]

Compound 217: Prepared in an analogous fashion to compound 214 using D-erythronolactone as the starting material. LCMS (C-18; 5-95 H₂O/MeCN): ELSD (5.08 min), UV (peak at 4.958 min), positive mode: m/z=407 [M+H]⁺; negative mode: m/z=405 [M−H]⁻C₂₅H₂₆O₅ (406).

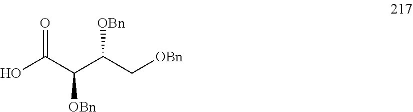

Compound 218: To a solution of compound 214 (3 eq) in anhydrous DMF was added HATU (3.3 eq) and DIPEA (5 eq). The mixture was stirred at ambient temperature for 15 minutes followed by addition of compound 78 (1 eq). The mixture was stirred at ambient temperature for 12 h. The solvent was removed in vacuo and the residue was purified by HPLC to afford compound 218.

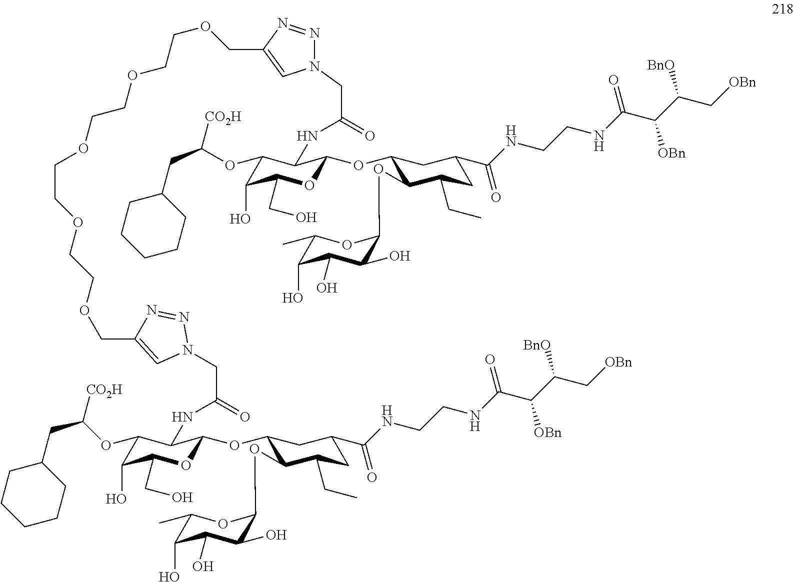

### Example 174

### Prophetic Synthesis of Multimeric Compound 219

Compound 219: Compound 218 is dissolved in methanol and degassed. To this solution is added Pd(OH)₂/C. The reaction mixture is vigorously stirred under a hydrogen atmosphere for 12 hours. The reaction mixture is filtered through a Celite pad. The filtrate is concentrated under reduced pressure to give compound 219.

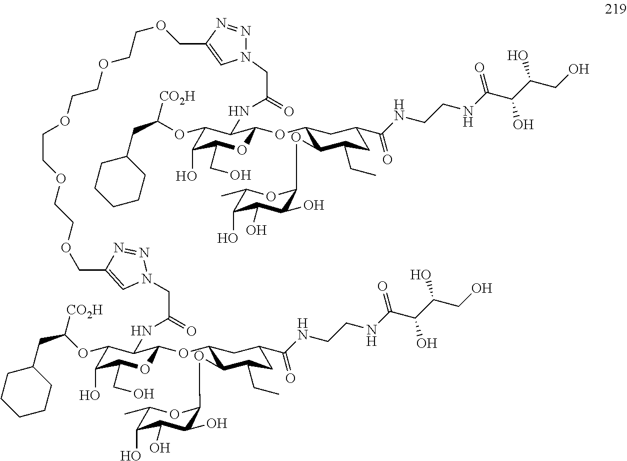

### Example 175

### Synthesis of Multimeric Compound 220

Compound 220: A solution of the sulfur trioxide pyridine complex (100 eq) and compound 219 (1 eq) in pyridine was stirred at 67° C. for 1 h. The reaction mixture was concentrated under vacuum. The resulting solid was dissolved in water and cooled to 0° C. A 1N solution of NaOH was then added slowly until pH-10 and the latter was freeze dried. The resulting residue was purified by Gel Permeation (water as eluent). The collected fractions were lyophilised to give compound 220.

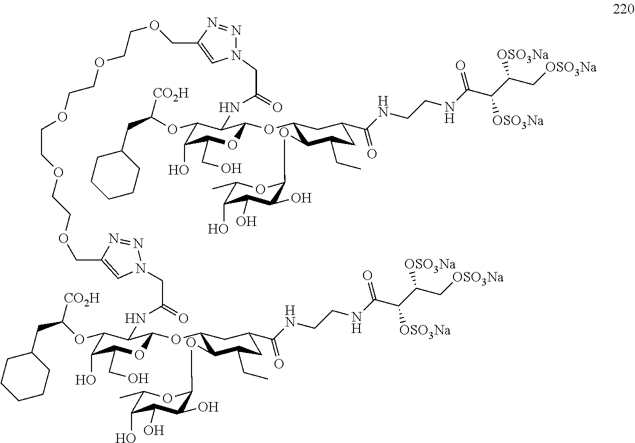

### Example 176

### Prophetic Synthesis of Multimeric Compound 221

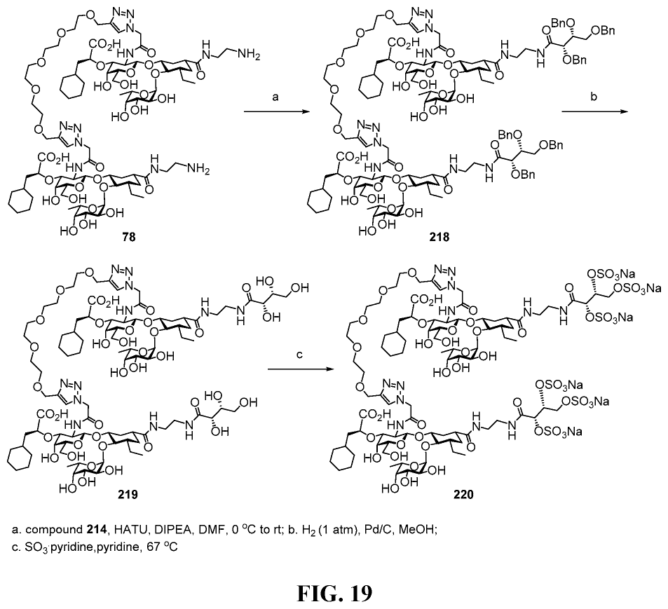

Compound 221: Compound 221 can be prepared in an analogous fashion to FIG. 19 by replacing compound 214 with compound 215.

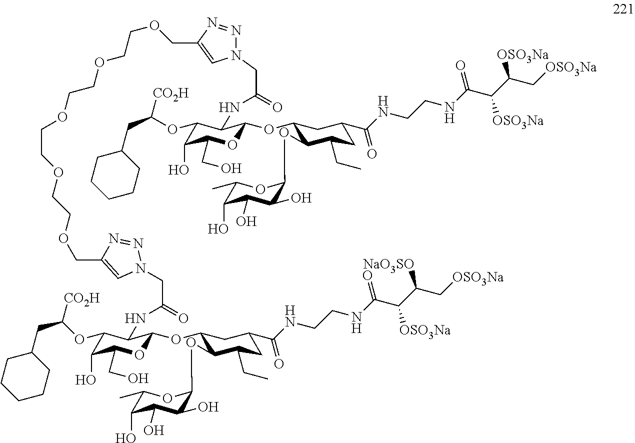

### Example 177

### Prophetic Synthesis of Multimeric Compound 222

Compound 222: Compound 222 can be prepared in an analogous fashion to FIG. 19 by replacing compound 214 with compound 216.

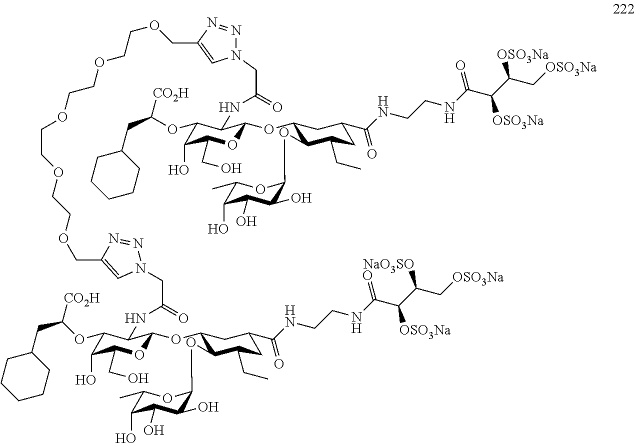

### Example 178

### Prophetic Synthesis of Multimeric Compound 223

Compound 223: Compound 223 can be prepared in an analogous fashion to FIG. 19 by replacing compound 214 with compound 217.

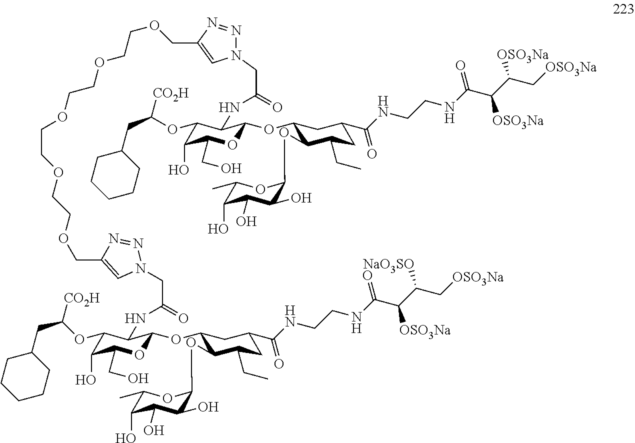

### Example 179

### Synthesis of Multimeric Compound 224

Compound 224: To a solution of compound 78 in anhydrous DMSO was added a drop of DIPEA and the solution was stirred at room temperature until a homogeneous solution was obtained. A solution of succinic anhydride (2.2 eq) in anhydrous DMSO was added and the resulting solution was stirred at room temperature overnight. The solution was lyophilized to dryness and the crude product was purified by HPLC to give compound 224.

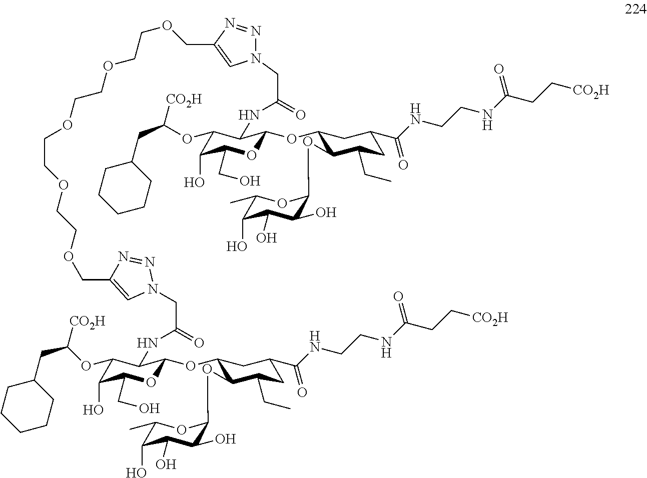

### Example 180

### Prophetic Synthesis of Multimeric Compound 225

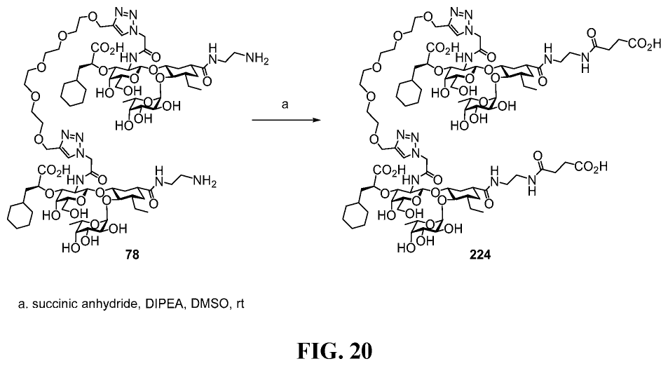

Compound 225: Compound 225 can be prepared in an analogous fashion to FIG. 20 substituting glutaric anhydride for succinic anhydride.

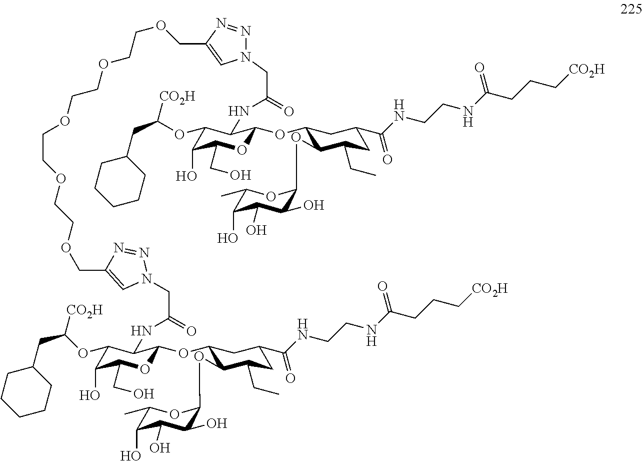

### Example 181

### Prophetic Synthesis of Multimeric Compound 226

Compound 226: Compound 226 can be prepared in an analogous fashion to FIG. 20 substituting compound 87 for compound 78.

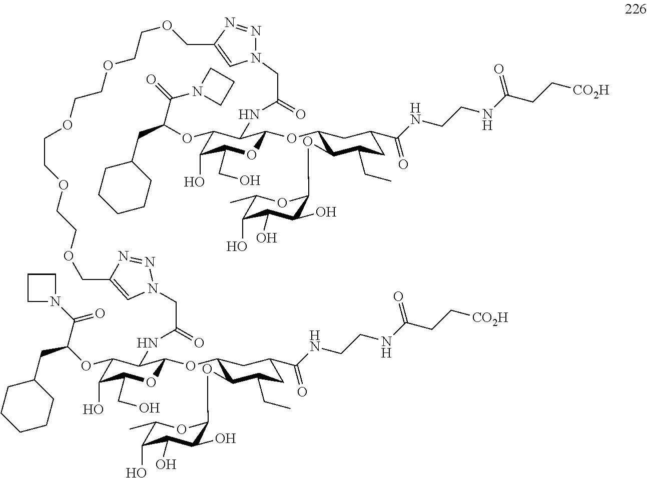

### Example 182

### Prophetic Synthesis of Multimeric Compound 227

Compound 227: Compound 227 can be prepared in an analogous fashion to FIG. 20 substituting phthalic anhydride for succinic anhydride.

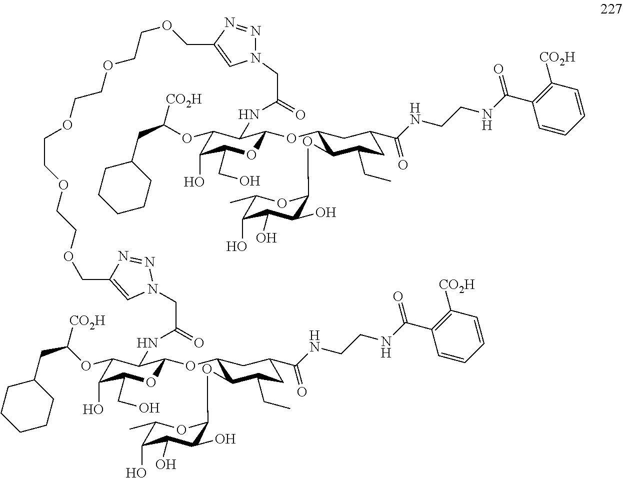

### Example 183

### Prophetic Synthesis of Multimeric Compound 228

Compound 228: Compound 228 can be prepared in an analogous fashion to FIG. 20 using compound 83 in place of compound 78.

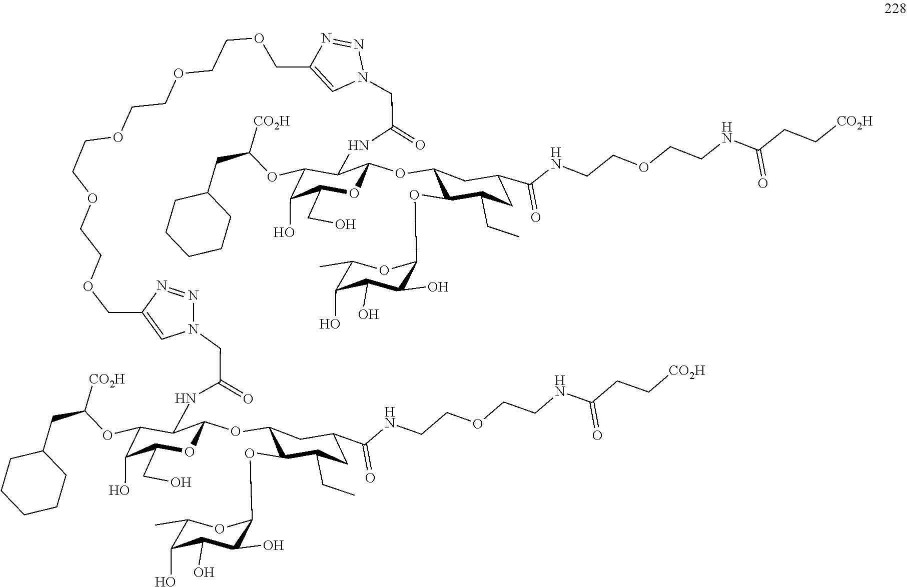

### Example 184

### Prophetic Synthesis of Multimeric Compound 229

Compound 229: Compound 229 can be prepared in an analogous fashion to FIG. 20 using compound 87 in place of compound 78.

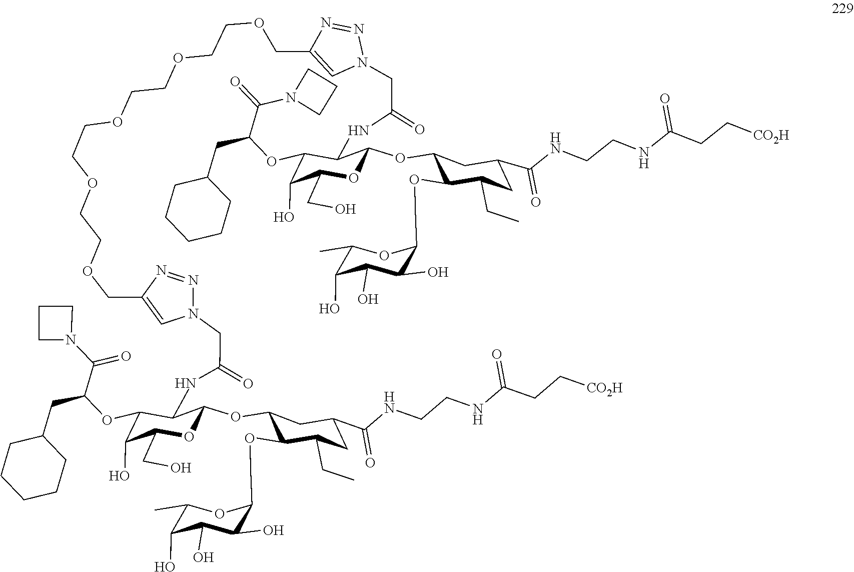

### Example 185

### Prophetic Synthesis of Multimeric Compound 245

Compound 231: A mixture of compounds 230 (preparation described in Schwizer, et. al., Chem. Eur. J., 2012, 18, 1342) and compound 2 (preparation described in WO 2013/096926) (1.7 eq) is azeotroped 3 times from toluene. The mixture is dissolved in DCM under argon and cooled on an ice bath. To this solution is added boron trifluoride etherate (1.5 eq). The reaction mixture is stirred 12 hours at room temperature. The reaction is quenched by the addition of triethylamine (2 eq). The reaction mixture is transferred to a separatory funnel and washed 1 time with half saturated sodium bicarbonate solution and 1 time with water. The organic phase is dried over sodium sulfate, filtered, and concentrated. The residue is purified by flash chromatography to afford compound 231.

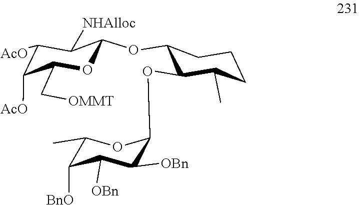

Compound 232: Compound 231 is dissolved in methanol at room temperature. A solution of sodium methoxide in methanol (0.1 eq) is added and the reaction mixture stirred overnight at room temperature. The reaction mixture is quenched by the addition of acetic acid. The reaction mixture is diluted with ethyl acetate, transferred to a separatory funnel and washed 2 times with water. The organic phase is dried over magnesium sulfate, filtered and concentrated. The residue is separated by flash chromatography to afford compound 232.

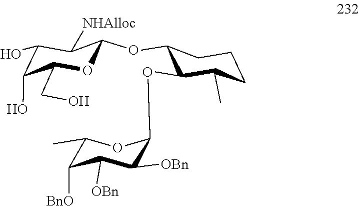

Compound 233: To a solution of compound 232 in dichloromethane cooled on an ice bath is added DABCO (1.5 eq) followed by monomethyoxytrityl chloride (1.2 eq). The reaction mixture is stirred overnight allowing to warm to room temperature. The reaction mixture is concentrated and the residue is purified by flash chromatography to afford compound 233.

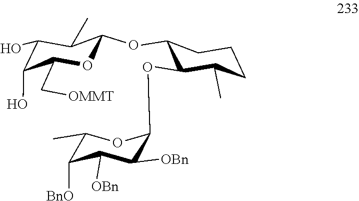

Compound 234: To a solution of compound 233 in methanol is added dibutyltin oxide (1.1 eq). The reaction mixture is refluxed for 3 hours then concentrated. The residue is suspended in DME. To this suspension is added compound 6 (preparation described in Thoma et. al. J. Med. Chem., 1999, 42, 4909) (1.5 eq) followed by cesium fluoride (1.2 eq). The reaction mixture is stirred at room temperature overnight. The reaction mixture is diluted with ethyl acetate, transferred to a separatory funnel, and washed with water. The organic phase is dried over sodium sulfate, filtered and concentrated. The residue is purified by flash chromatography to afford compound 234.

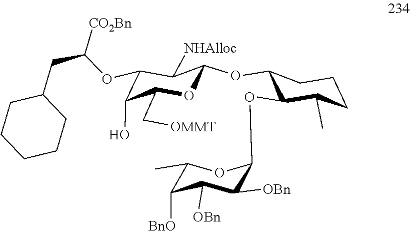

Compound 235: To a degassed solution of compound 234 in anhydrous DCM at 0° C. is added Pd(PPh₃)₄ (0.1 eq), Bu₃SnH (1.1 eq) and N-trifluoroacetyl glycine anhydride (2.0 eq) (preparation described in Chemische Berichte (1955), 88(1), 26). The resulting solution is stirred for 12 hrs allowing the temperature to increase to room temperature. The reaction mixture is diluted with DCM, transferred to a separatory funnel, and washed with water. The organic phase is dried over Na₂SO₄, then filtered and concentrated. The residue is purified by flash chromatography to afford compound 235.

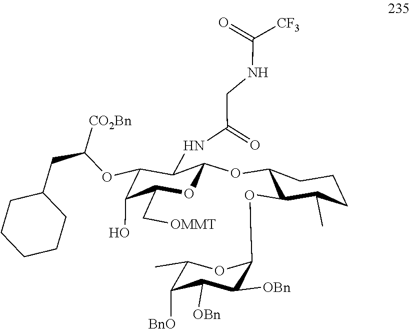

Compound 236: Compound 235 is dissolved in methanol and degassed. To this solution is added Pd(OH)₂/C. The reaction mixture is vigorously stirred under a hydrogen atmosphere for 12 hours. The reaction mixture is filtered through a Celite pad. The filtrate is concentrated under reduced pressure to give compound 236.

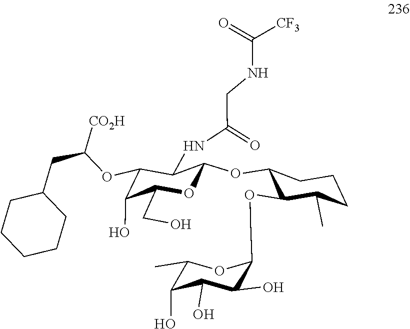

Compound 237: Compound 236 is dissolved in methanol at room temperature. A solution of sodium methoxide in methanol (1.1 eq) is added and the reaction mixture stirred overnight at room temperature. The reaction mixture is quenched by the addition of acetic acid. The reaction mixture is concentrated. The residue is separated by C-18 reverse phase chromatography to afford compound 237.

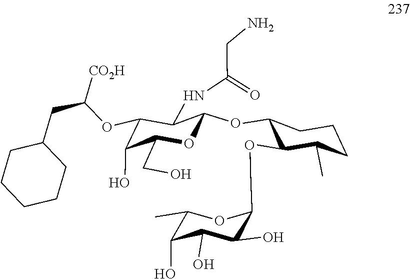

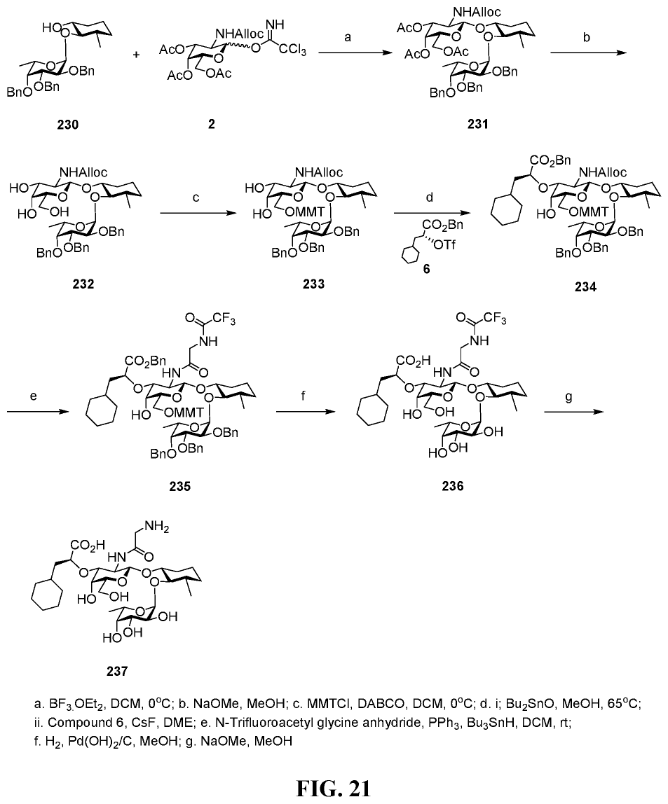

Compound 238: Compound 238 can be prepared in an analogous fashion to FIG. 21 by substituting (acetylthio)acetyl chloride for N-trifluoroacetyl glycine anhydride in step e.

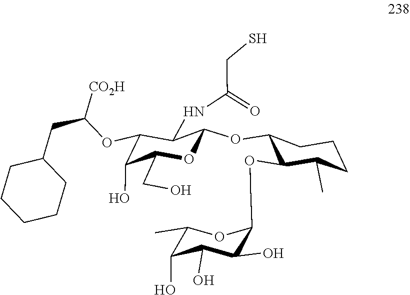

Compound 239: Compound 239 can be prepared in an analogous fashion to FIG. 21 by substituting the vinylcyclohexyl analog of compound 230 (preparation described in Schwizer, et. al., Chem. Eur. J., 2012, 18, 1342) for compound 230 in step a.

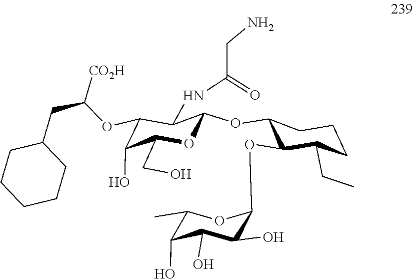

Compound 240: Compound 236 is dissolved in DMF and cooled on an ice bath. Diisopropylethylamine (1.5 eq) is added followed by HATU (1.1 eq). The reaction mixture is stirred 15 minutes on the ice bath then azetidine (2 eq) is added. The ice bath is removed and the reaction mixture is stirred overnight at room temperature. The solvent is removed under reduced pressure and the residue is separated by flash chromatography to afford compound 240.

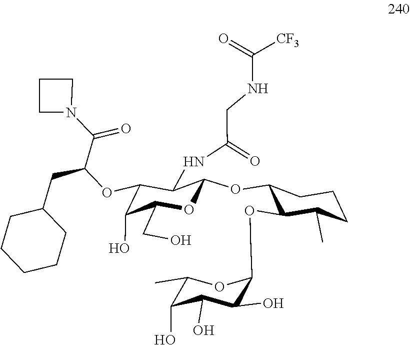

Compound 241: Compound 240 is dissolved in methanol at room temperature. A solution of sodium methoxide in methanol (0.3 eq) is added and the reaction mixture stirred overnight at room temperature. The reaction mixture is quenched by the addition of acetic acid. The reaction mixture is concentrated. The residue is separated by C-18 reverse phase chromatography to afford compound 241.

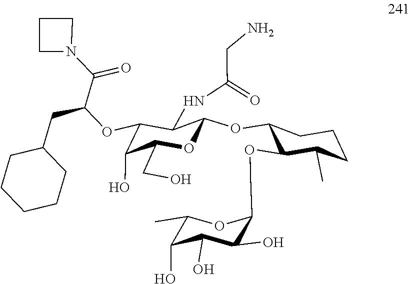

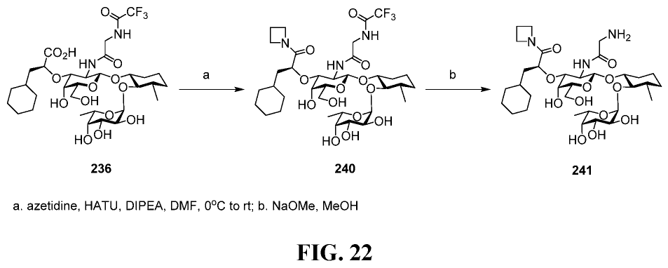

Compound 242: Compound 242 can be prepared in an analogous fashion to FIG. 22 by using methylamine in place of azetidine in step a.

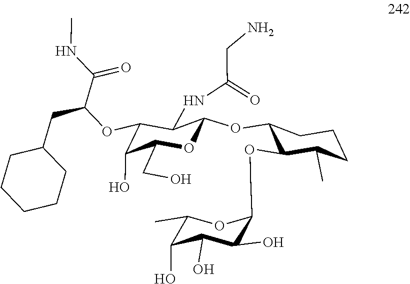

Compound 243: Compound 243 can be prepared in an analogous fashion to FIG. 22 by using dimethylamine in place of azetidine in step a.

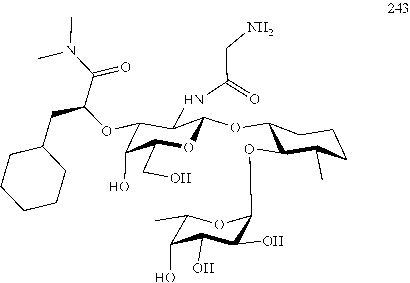

Compound 244: Compound 244 can be prepared in an analogous fashion to FIG. 22 by using the ethylcyclohexyl analog of compound 236 in place of compound 236 in step a.

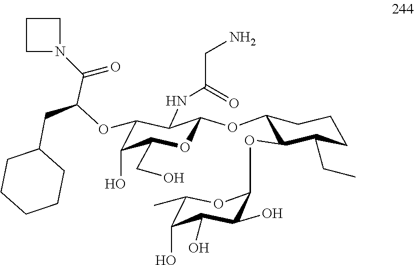

Compound 245: A solution of compound 20 (0.4 eq) in DMSO is added to a solution of compound 237 (1 eq) and DIPEA (10 eq) in anhydrous DMSO at room temperature. The resulting solution is stirred overnight. The reaction mixture is separated by reverse phase chromatography and the product lyophilized to give compound 245.

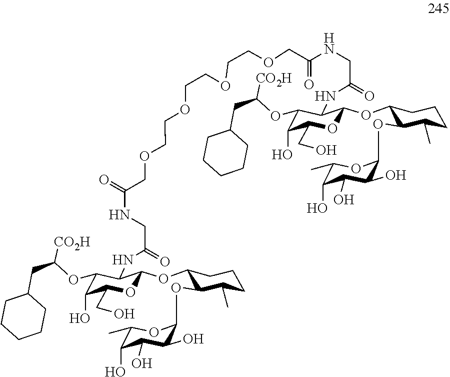

### Example 186

### Prophetic Synthesis of Multimeric Compound 246

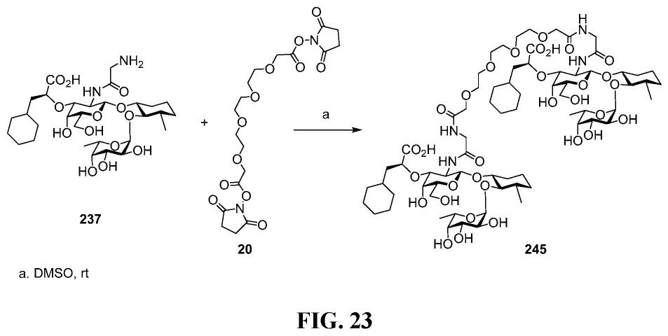

Compound 246: Compound 246 can be prepared in an analogous fashion to FIG. 23 by replacing compound 20 with PEG-11 diacetic acid di-NHS ester.

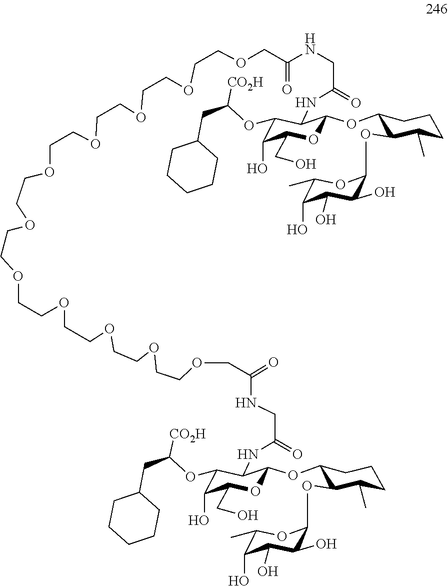

### Example 187

### Prophetic Synthesis of Multimeric Compound 247

Compound 247: Compound 247 can be prepared in an analogous fashion to FIG. 23 by replacing compound 20 with PEG-15 diacetic acid di-NHS ester.

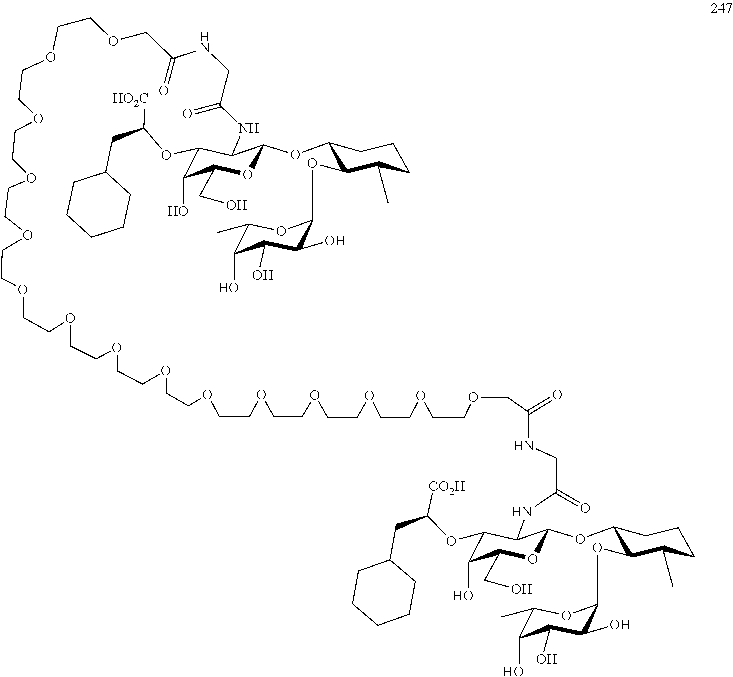

### Example 188

### Prophetic Synthesis of Multimeric Compound 248

Compound 248: Compound 248 can be prepared in an analogous fashion to FIG. 23 by replacing compound 20 with ethylene glycol diacetic acid di-NHS ester.

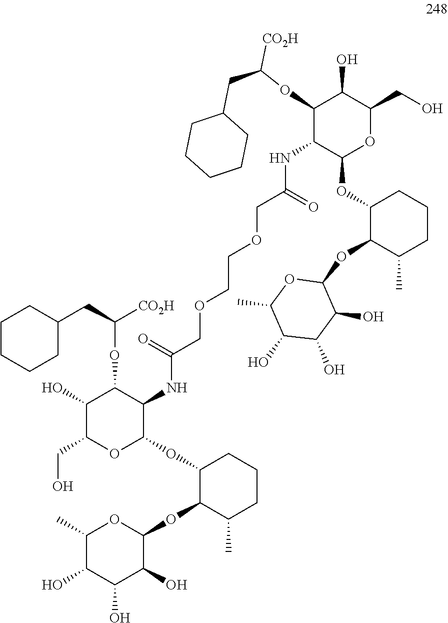 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3CCC[C@H](C)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H](O)[C@H](O)[C@@H]1O]

### Example 189

### Prophetic Synthesis of Multimeric Compound 249

Compound 249: Compound 249 can be prepared in an analogous fashion to FIG. 23 by replacing compound 20 with 3,3′-[[2,2-bis[[3-[(2,5-dioxo-1-pyrrolidinyl)oxy]-3-oxopropoxy]methyl]-1,3-propanediyl]bis(oxy)]bis-1,1′-bis(2,5-dioxo-1-pyrrolidinyl)-propanoic acid ester.

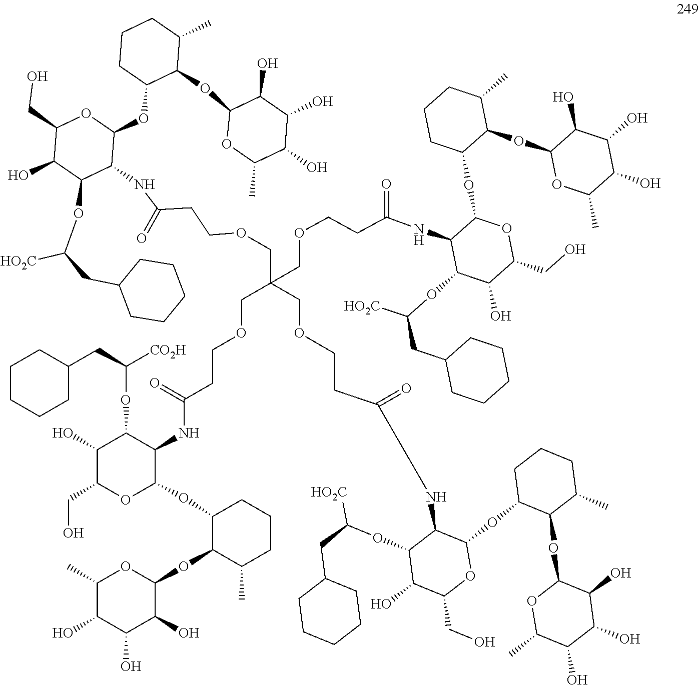

### Example 190

### Prophetic Synthesis of Multimeric Compound 250

Compound 250: Compound 250 can be prepared in an analogous fashion to FIG. 23 by replacing compound 237 with compound 239.

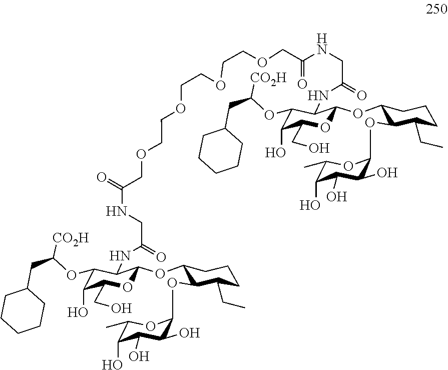

### Example 191

### Prophetic Synthesis of Multimeric Compound 251

Compound 251: Compound 251 can be prepared in an analogous fashion to FIG. 23 by replacing compound 237 with compound 241 and compound 20 with PEG-11 diacetic acid di-NHS ester.

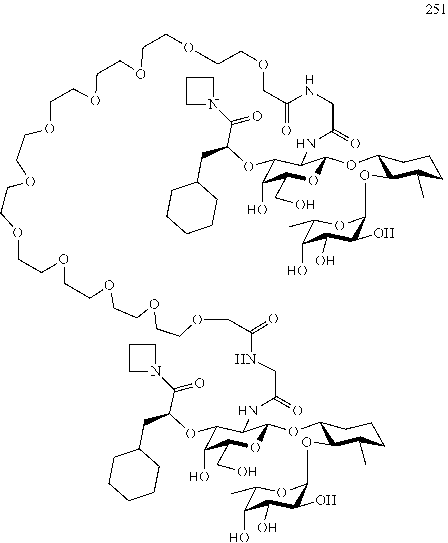

### Example 192

### Prophetic Synthesis of Multimeric Compound 252

Compound 252: Compound 252 can be prepared in an analogous fashion to FIG. 23 by replacing compound 237 with compound 242.

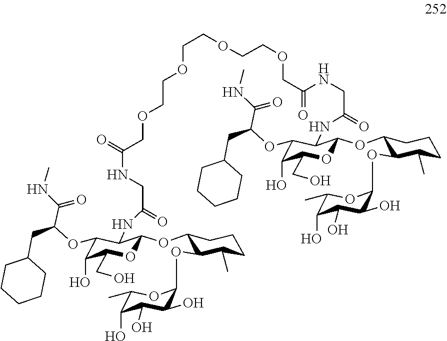

### Example 193

### Prophetic Synthesis of Multimeric Compound 253

Compound 253: Compound 253 can be prepared in an analogous fashion to FIG. 23 by replacing compound 237 with compound 243 and compound 20 with ethylene glycol diacetic acid di-NHS ester.

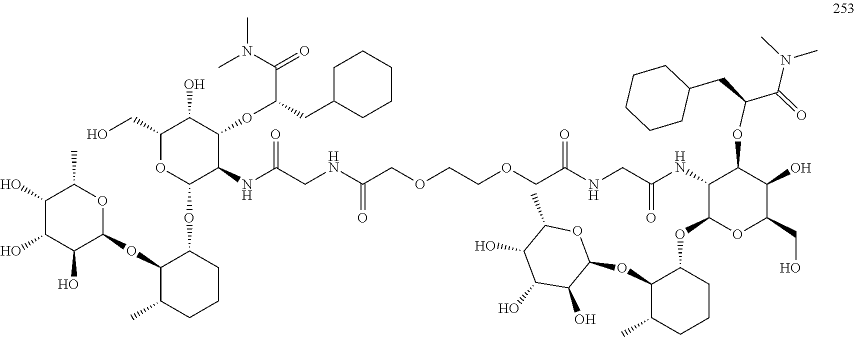 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)N(C)C)[C@H]2NC(=O)CNC(=O)COCCOCC(=O)NCC(=O)N[C@H]2[C@H](O[C@@H]3CCC[C@H](C)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N(C)C)[C@@H](O)[C@H](O)[C@@H]1O]

### Example 194

### Prophetic Synthesis of Multimeric Compound 254

Compound 254: Compound 254 can be prepared in an analogous fashion to FIG. 23 by replacing compound 237 with compound 244 and compound 20 with PEG-11 diacetic acid di-NHS ester.

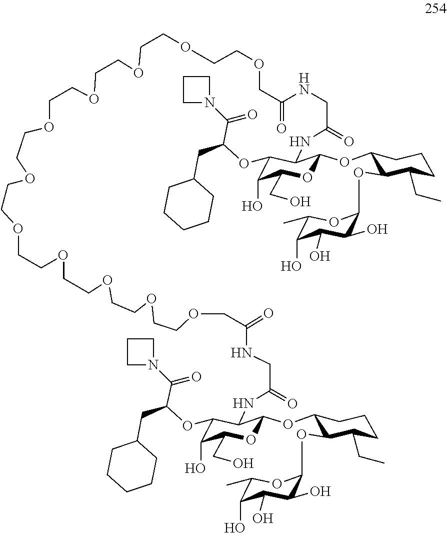

### Example 195

### Prophetic Synthesis of Multimeric Compound 255

Compound 255: Compound 255 can be prepared in an analogous fashion to FIG. 23 by replacing compound 237 with compound 241 and compound 20 with 1,1′-[oxybis[(1-oxo-2,1-ethanediyl)oxy]]bis-2,5-pyrrolidinedione.

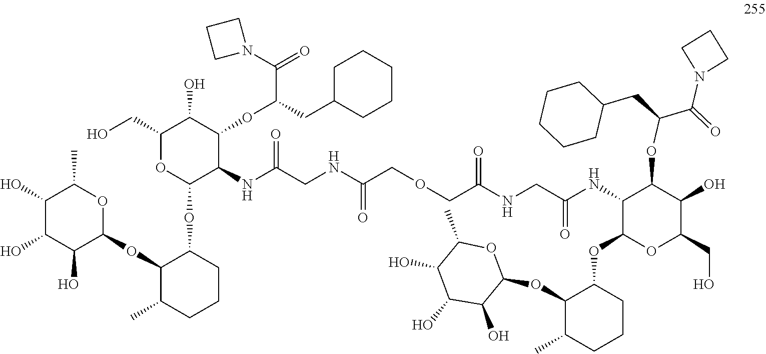 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)N3CCC3)[C@H]2NC(=O)CNC(=O)COCC(=O)NCC(=O)N[C@H]2[C@H](O[C@@H]3CCC[C@H](C)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCC2)[C@@H](O)[C@H](O)[C@@H]1O]

### Example 196

### Prophetic Synthesis of Multimeric Compound 256

Compound 256: Compound 256 can be prepared in an analogous fashion to FIG. 23 by replacing compound 237 with compound 244 and compound 20 with 1,1′-[oxybis[(1-oxo-2,1-ethanediyl)oxy]]bis-2,5-pyrrolidinedione.

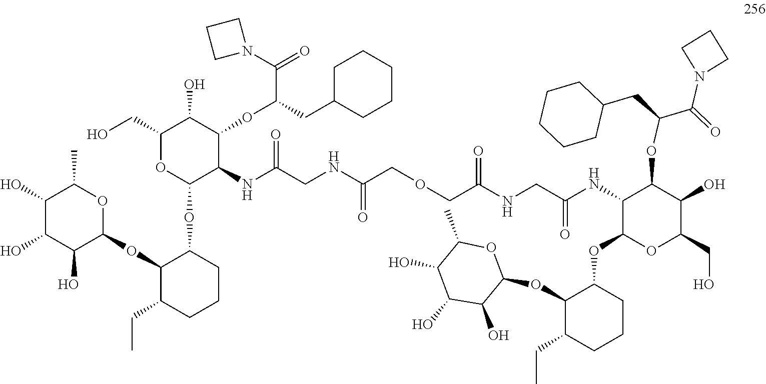 [SMILES: CC[C@H]1CCC[C@@H](O[C@@H]2O[C@H](CO)[C@H](O)[C@H](O[C@@H](CC3CCCCC3)C(=O)N3CCC3)[C@H]2NC(=O)CNC(=O)COCC(=O)NCC(=O)N[C@H]2[C@H](O[C@@H]3CCC[C@H](CC)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCC2)[C@@H]1O[C@@H]1O[C@@H](C)[C@@H](O)[C@@H](O)[C@@H]1O]

### Example 197

### Prophetic Synthesis of Multimeric Compound 257

Compound 257: To a solution of compound 238 in MeOH at room temperature is added compound 35 followed by cesium acetate (2.5 eq). The reaction mixture is stirred at room temperature until completion. The solvent is removed under reduced pressure. The product is purified by reverse phase chromatography to give compound 257.

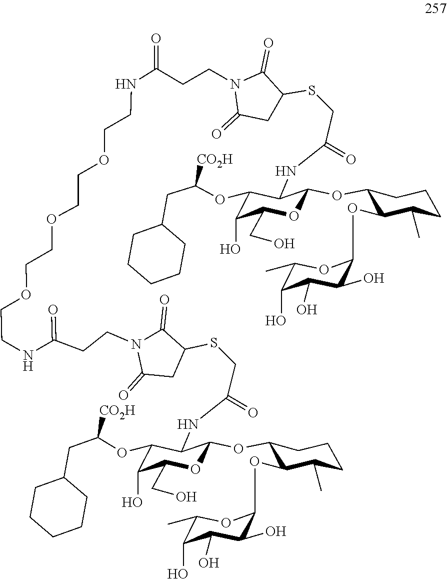

### Example 198

### Prophetic Synthesis of Multimeric Compound 258

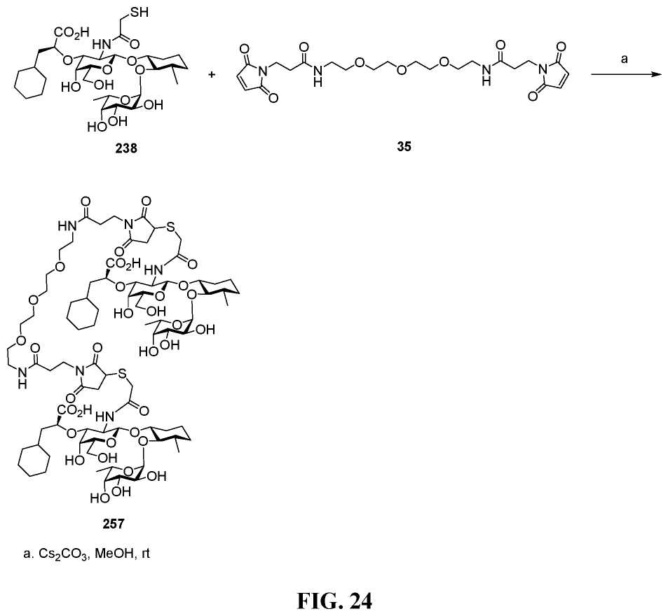

Compound 258: Compound 258 can be prepared in an analogous fashion to FIG. 24 by substituting PEG-6-bis maleimidoylpropionamnide for compound 35.

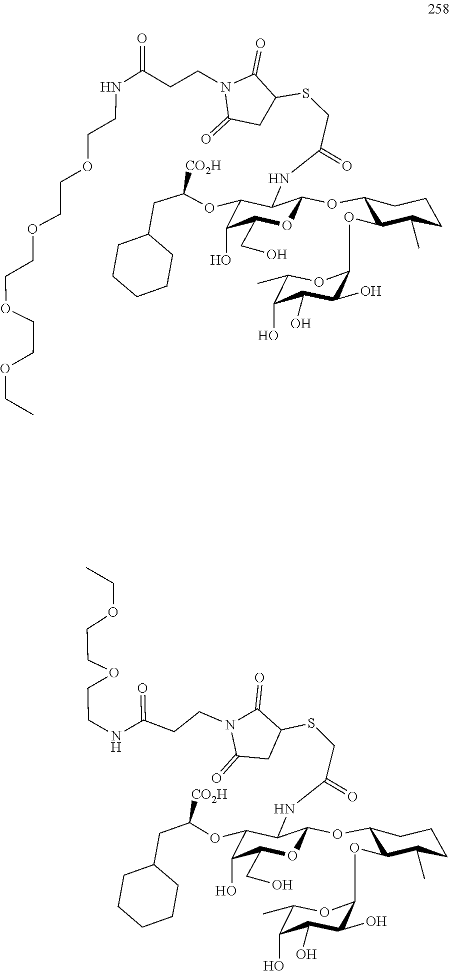

### Example 199

### Prophetic Synthesis of Multimeric Compound 259

Compound 259: Compound 259 can be prepared in an analogous fashion to FIG. 24 by substituting compound 35 for, 1,1′-[[2,2-bis[[3-(2,5-dihydro-2,5-dioxo-1H-pyrrol-1-yl)propoxy]methyl]-1,3-propanediyl]bis(oxy-3,1-propanediyl)]bis-1H-pyrrole-2,5-dione.

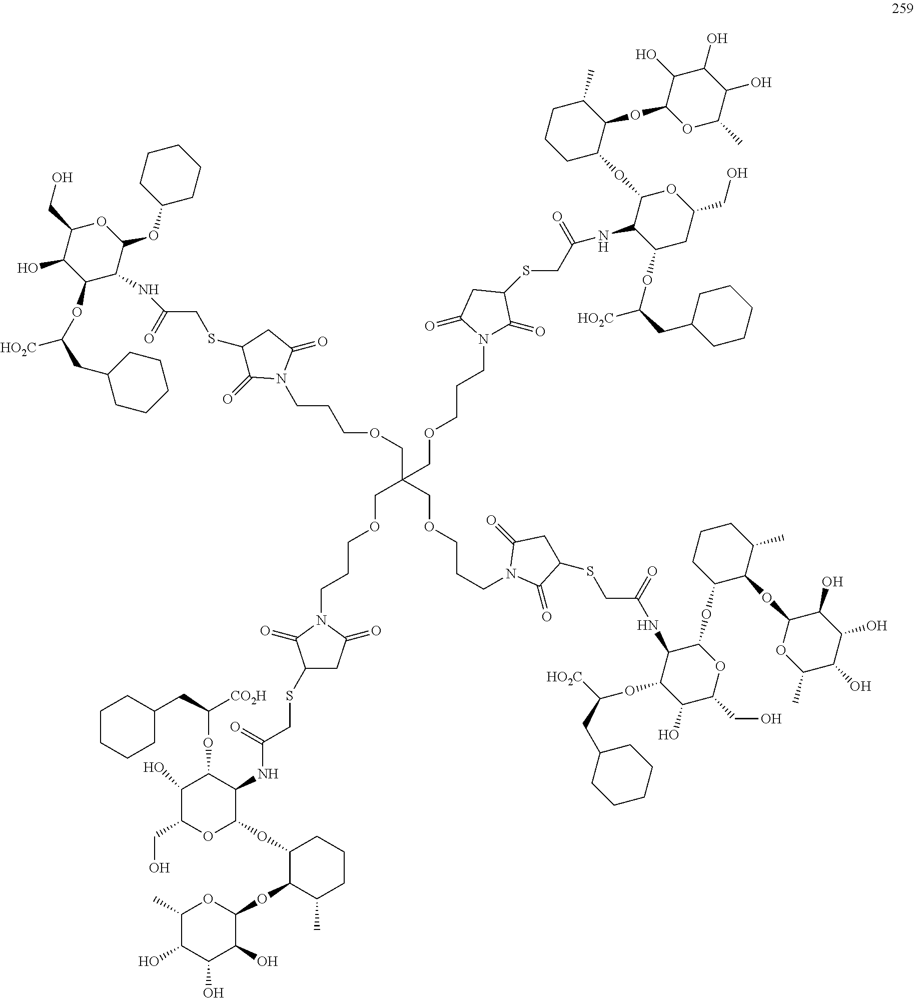

### Example 200

### Prophetic Synthesis of Multimeric Compound 261

Compound 260: To a degassed solution of compound 234 in anhydrous DCM at 0° C. is added Pd(PPh₃)₄ (0.1 eq), Bu₃SnH (1.1 eq) and azidoacetic anhydride (2.0 eq). The ice bath is removed and the solution is stirred for 12 hrs under a N₂ atmosphere at room temperature. The reaction mixture is diluted with DCM, washed with water, dried over Na₂SO₄, then concentrated. The crude product is purified by column chromatography to give compound 260.

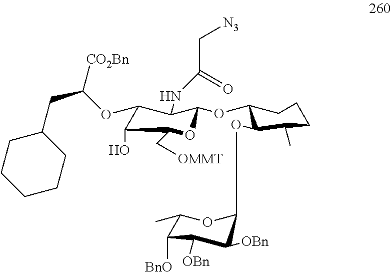

Compound 261: A solution of bis-propagyl PEG-5 (compound 43) and compound 260 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred 12 hrs at 70° C. The solution is cooled to room temperature and concentrated under reduced pressure. The crude product is purified by chromatography to give compound 261.

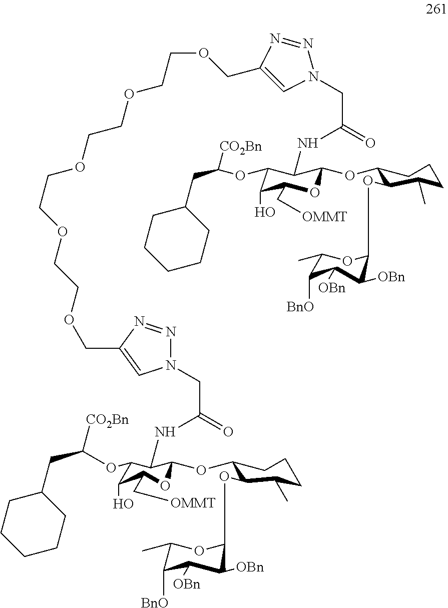

### Example 201

### Prophetic Synthesis of Multimeric Compound 262

Compound 262: Compound 261 is dissolved in MeOH and hydrogenated in the presence of Pd(OH)₂ (20 wt %) at 1 atm of H₂ gas pressure for 24 hrs at room temperature. The solution is filtered through a Celite pad. The filtrate is concentrated to give compound 262.

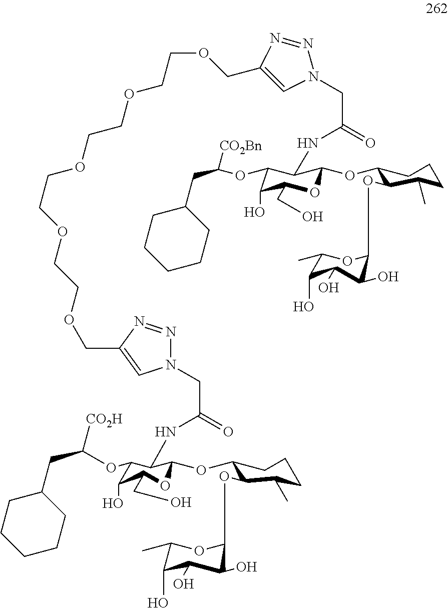

### Example 202

### Prophetic Synthesis of Multimeric Compound 263

Compound 263: Compound 262 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by reverse phase chromatography to afford compound 263.

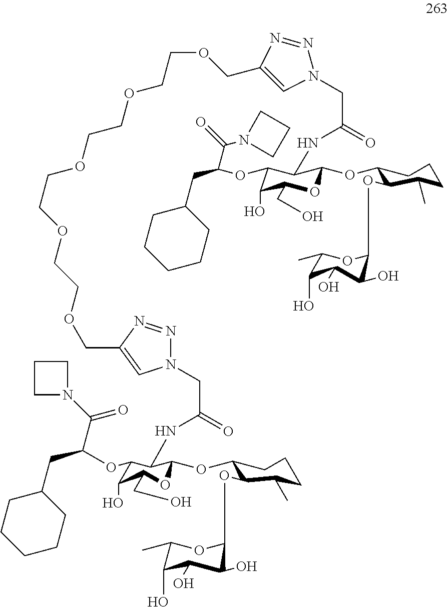

### Example 203

### Prophetic Synthesis of Multimeric Compound 264

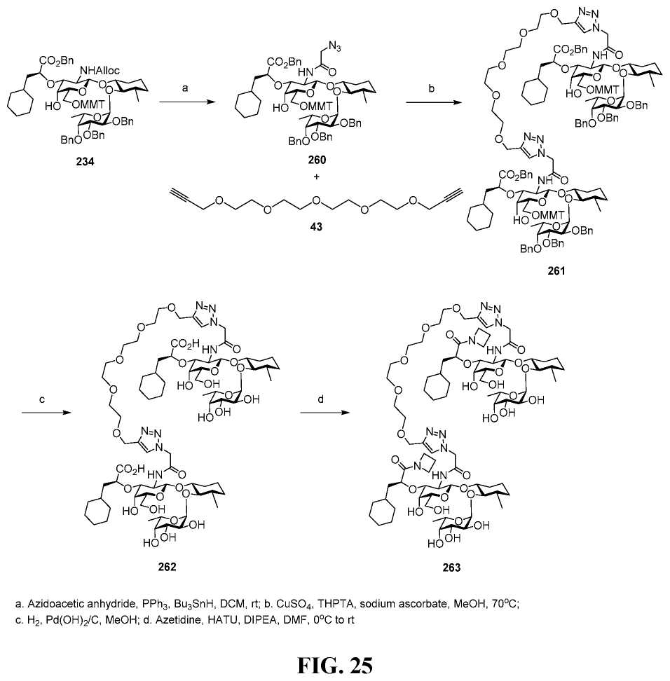

Compound 264: Compound 264 can be prepared in an analogous fashion to FIG. 25 using 4,7,10,13,16,19,22,25,28,31-decaoxatetratriaconta-1, 33-diyne in place of compound 43 in step b.

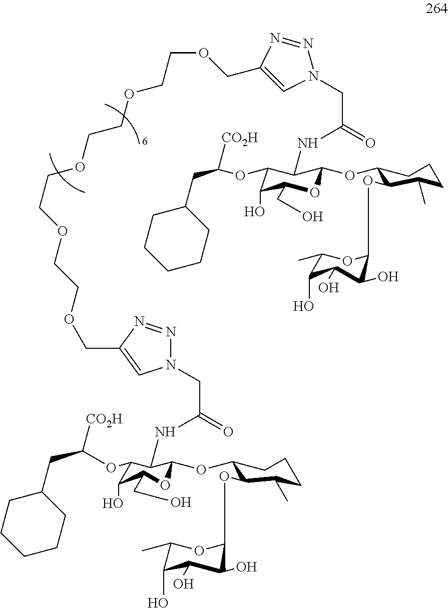

### Example 204

### Prophetic Synthesis of Multimeric Compound 265

Compound 265: Compound 265 can be prepared in an analogous fashion to FIG. 25 using 3,3′-[[2,2-bis[(2-propyn-1-yloxy)methyl]-1,3-propanediyl]bis(oxy)]bis-1-propyne in place of compound 43 in step b.

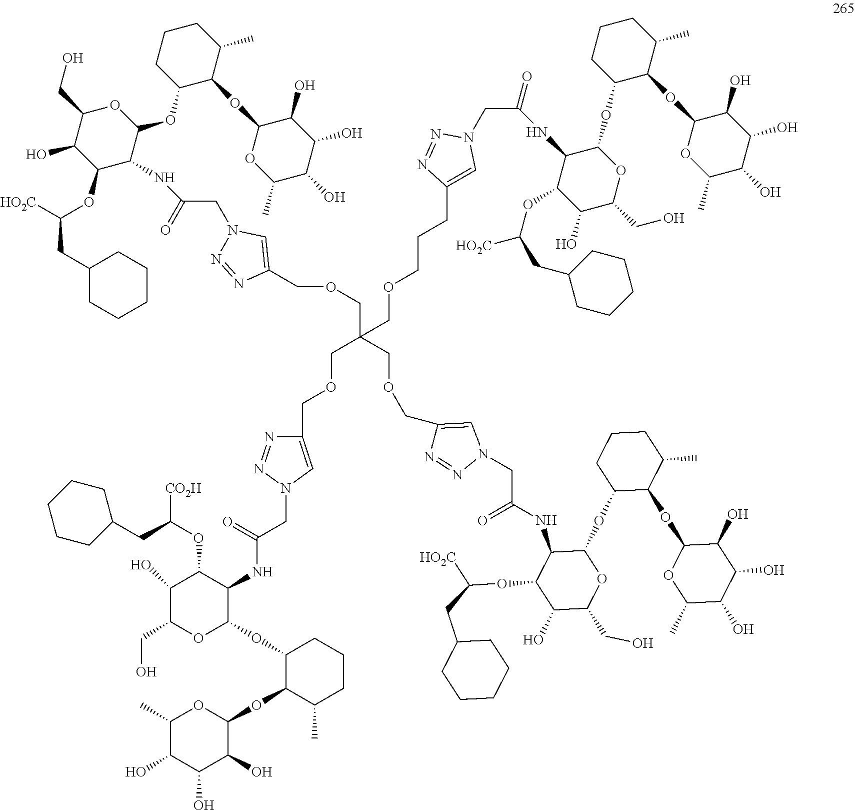

### Example 205

### Prophetic Synthesis of Multimeric Compound 266

Compound 266: Compound 266 can be prepared in an analogous fashion to FIG. 25 using 3,3′-[oxybis[[2,2-bis[(2-propyn-1-yloxy)methyl]-3,1-propanediyl]oxy]]bis-1-propyne in place of compound 43 in step b.

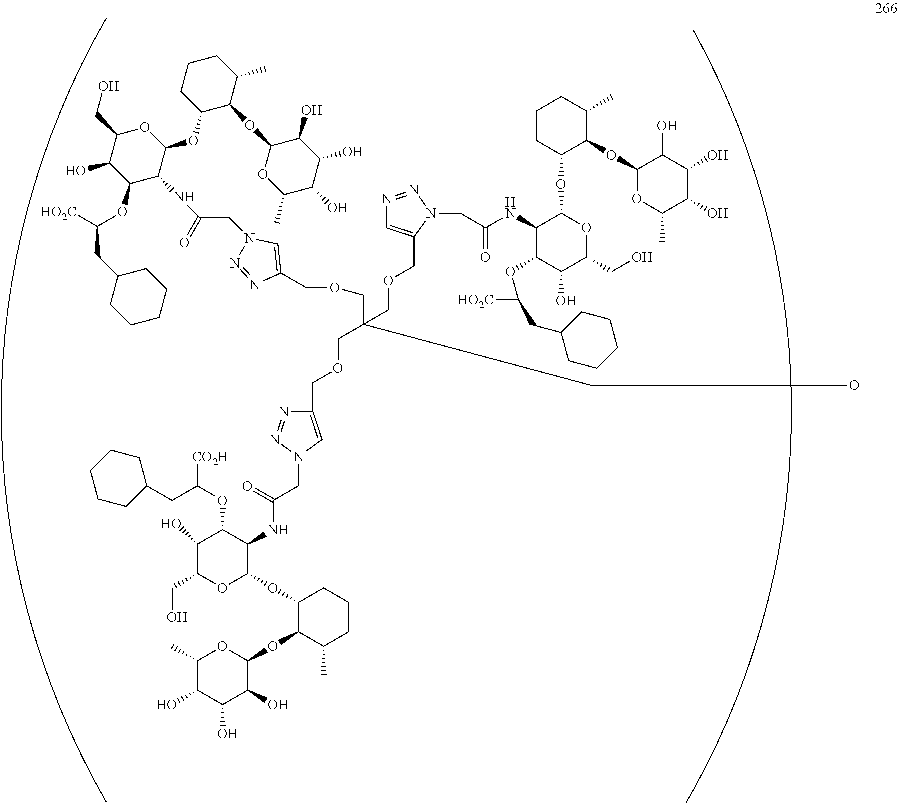

### Example 206

### Prophetic Synthesis of Multimeric Compound 267

Compound 267: Compound 267 can be prepared in an analogous fashion to FIG. 25 using ethylamine in place of azetidine in step d.

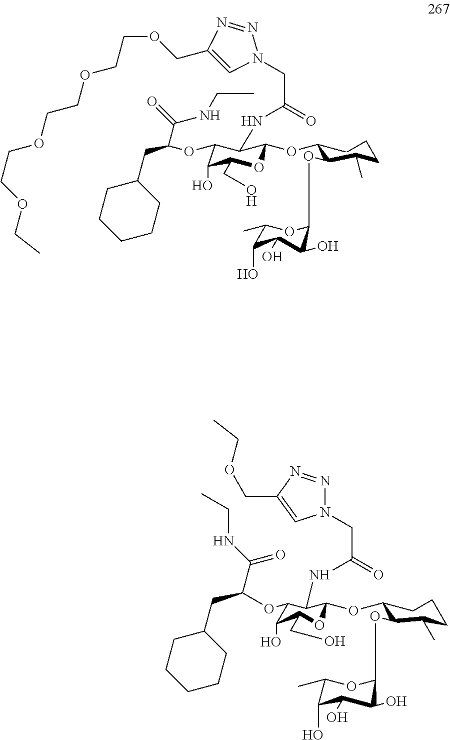

### Example 207

### Prophetic Synthesis of Multimeric Compound 268

Compound 268: Compound 268 can be prepared in an analogous fashion to FIG. 25 using dimethylamine in place of azetidine in step d.

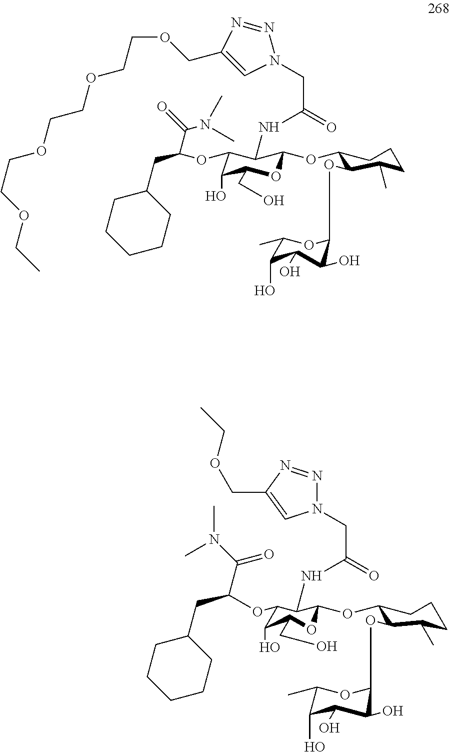

### Example 208

### Prophetic Synthesis of Multimeric Compound 269

Compound 269: Compound 269 can be prepared in an analogous fashion to FIG. 25 using the analog of compound 234 prepared from vinylcyclohexane in place of compound 234 in step a.

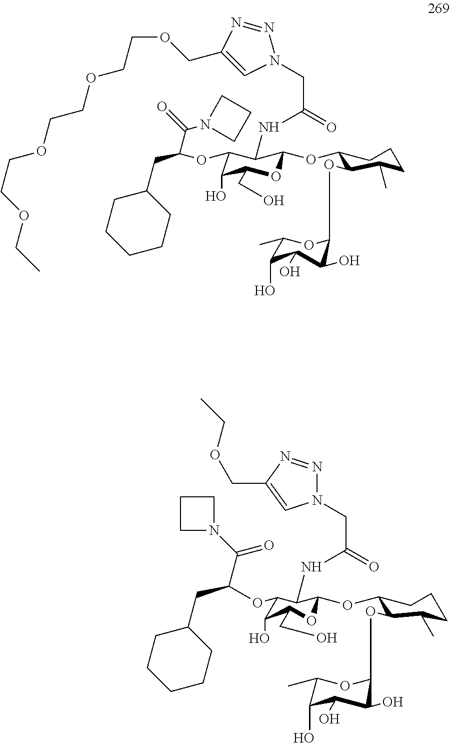

### Example 209

### Prophetic Synthesis of Multimeric Compound 270

Compound 270: Compound 270 can be prepared in an analogous fashion to FIG. 25 using propargyl ether in place of compound 43 in step b.

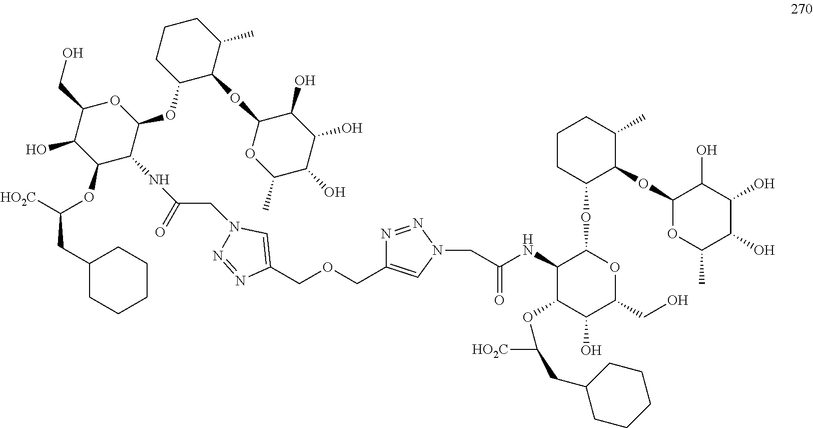 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)O)[C@H]2NC(=O)Cn2cc(COCc3cn(CC(=O)N[C@H]4[C@H](O[C@@H]5CCC[C@H](C)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)C(O)[C@H](O)[C@@H]1O]

### Example 210

### Prophetic Synthesis of Multimeric Compound 271

Compound 271: Compound 271 can be prepared in an analogous fashion to FIG. 25 using propargyl ether in place of compound 43 in step b.

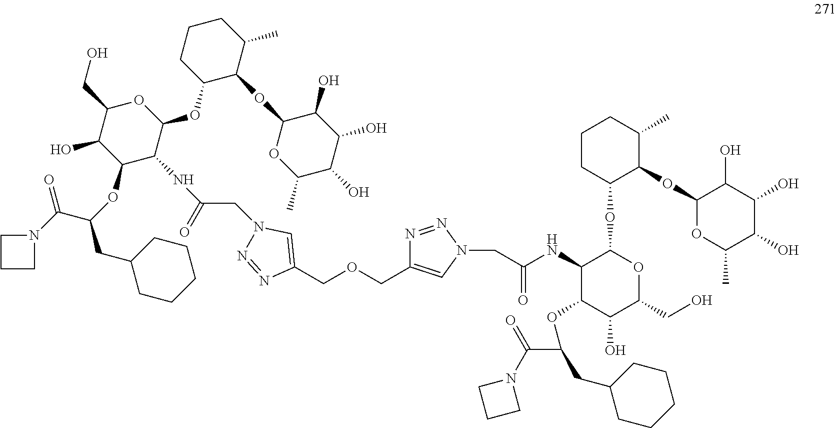 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)N3CCC3)[C@H]2NC(=O)Cn2cc(COCc3cn(CC(=O)N[C@H]4[C@H](O[C@@H]5CCC[C@H](C)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)N4CCC4)nn3)nn2)C(O)[C@H](O)[C@@H]1O]

### Example 211

### Prophetic Synthesis of Multimeric Compound 274

Compound 272: Activated powdered 4 Å molecular sieves are added to a solution of compound 230 and compound 63 (2 eq) in dry DCM under argon. The mixture is stirred for 2 hours at room temperature. Solid DMTST (1.5 eq) is added in 4 portions over 1.5 hours. The reaction mixture is stirred overnight at room temperature. The reaction mixture is filtered through Celite, transferred to a separatory funnel and washed two times with half saturated sodium bicarbonate and two times with water. The organic phase is dried over sodium sulfate, filtered and concentrated. The residue is separated by flash chromatography to afford compound 272.

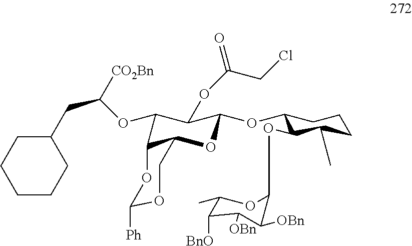

Compound 273: Compound 272 is dissolved in DMF. Sodium azide (1.5 eq) is added and the reaction mixture is stirred at 50° C. until completion. The reaction mixture is cooled to room temperature, diluted with ethyl acetate and transferred to a separatory funnel. The organic phase is washed 4 times with water then dried over sodium sulfate and concentrated. The residue is separated by column chromatography to afford compound 273.

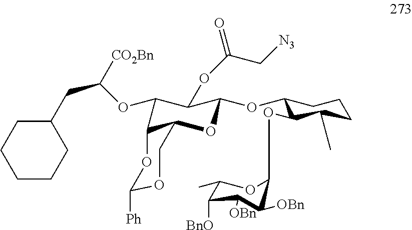

Compound 274: A solution of bispropagyl PEG-5 (compound 43) and compound 273 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred 12 hrs at 50° C. The solution is concentrated under reduced pressure. The crude product is purified by chromatography to give a compound 274.

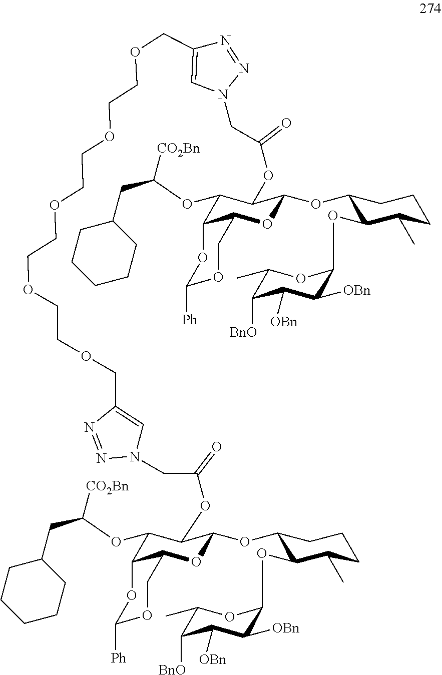

### Example 212

### Prophetic Synthesis of Multimeric Compound 275

Compound 275: To a solution of compound 274 in dioxane/water (4/1) is added Pd(OH)₂/C. The reaction mixture is stirred vigorously overnight under a hydrogen atmosphere. The reaction mixture is filtered through Celite and concentrated. The residue is purified by C-18 reverse phase column chromatography to afford compound 275.

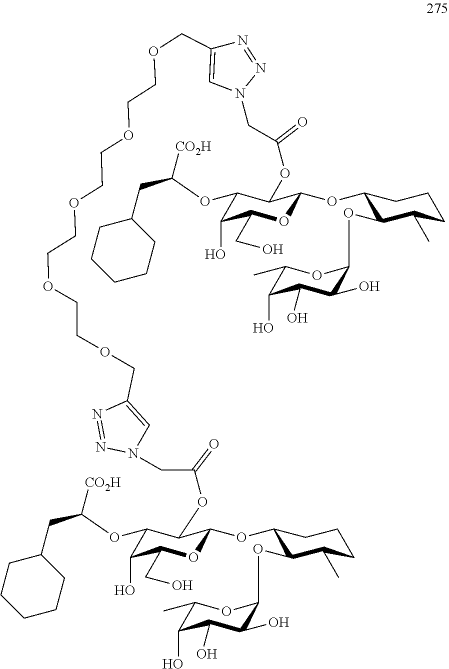

### Example 213

### Prophetic Synthesis of Multimeric Compound 276

Compound 276: Compound 275 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by reverse phase chromatography to afford compound 276.

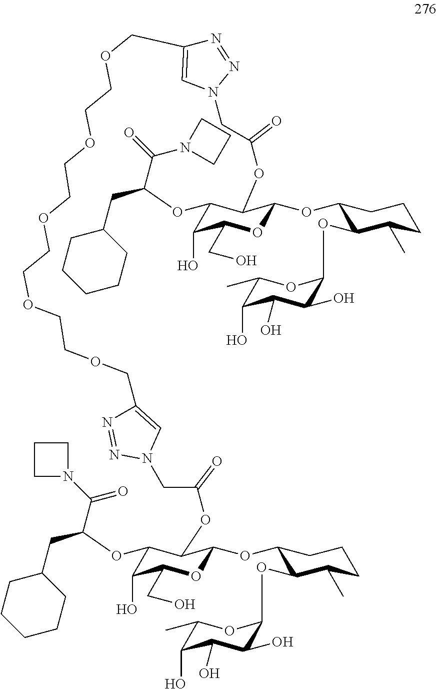

### Example 214

### Prophetic Synthesis of Multimeric Compound 277

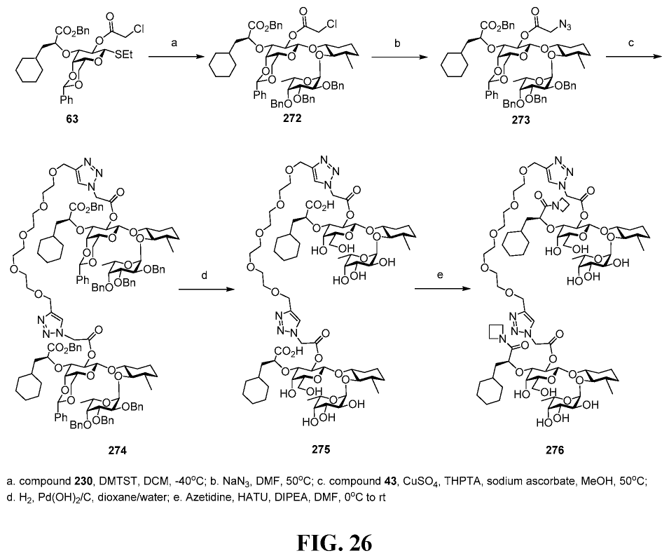

Compound 277: Compound 277 can be prepared in an analogous fashion to FIG. 26 by replacing compound 43 with PEG-8 bis propargyl ether in step c.

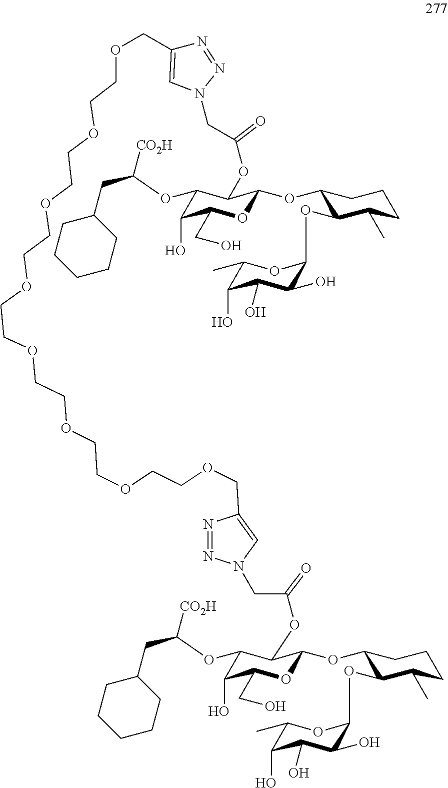

### Example 215

### Prophetic Synthesis of Multimeric Compound 278

Compound 278: Compound 278 can be prepared in an analogous fashion to FIG. 26 by replacing compound 43 with ethylene glycol bis propargyl ether in step c.

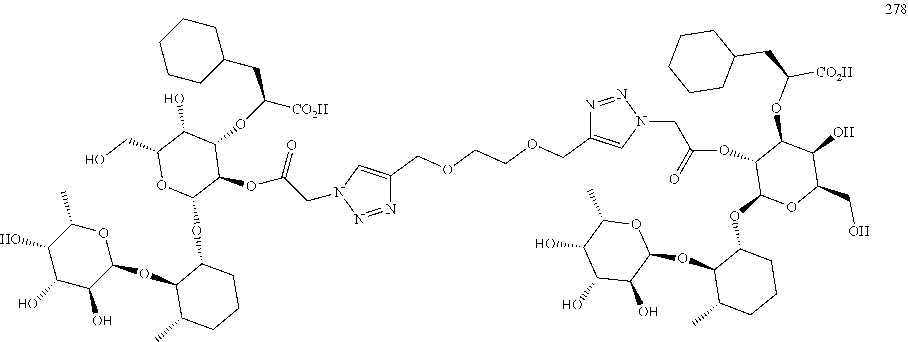 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)O)[C@H]2OC(=O)Cn2cc(COCCOCc3cn(CC(=O)O[C@H]4[C@H](O[C@@H]5CCC[C@H](C)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 216

### Prophetic Synthesis of Multimeric Compound 279

Compound 279: Compound 279 can be prepared in an analogous fashion to FIG. 26 using 3,3′-[[2,2-bis[(2-propyn-1-yloxy)methyl]-1,3-propanediyl]bis(oxy)]bis-1-propyne in place of compound 43 in step c.

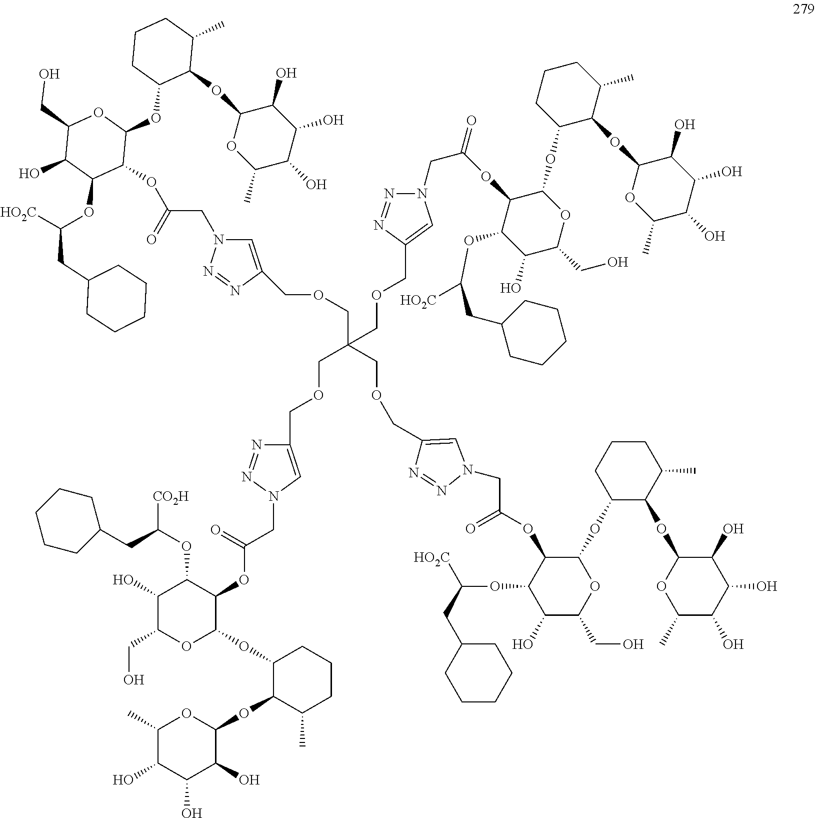

### Example 217

### Prophetic Synthesis of Multimeric Compound 280

Compound 280: Compound 280 can be prepared in an analogous fashion to FIG. 26 using propargyl ether in place of compound 43 in step c.

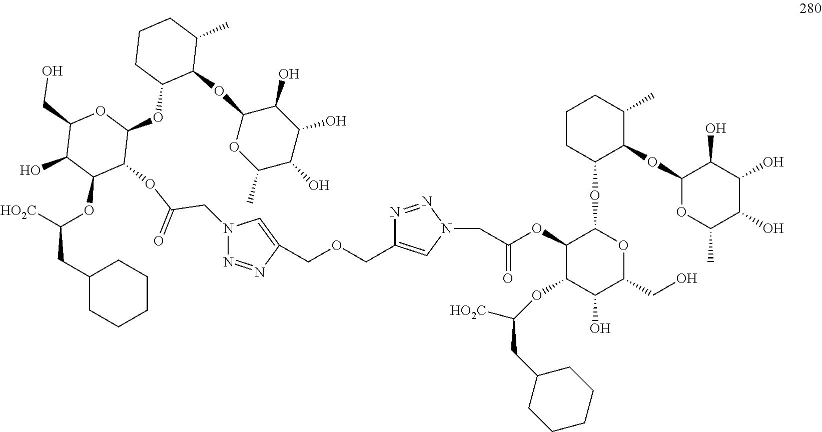 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)O)[C@H]2OC(=O)Cn2cc(COCc3cn(CC(=O)O[C@H]4[C@H](O[C@@H]5CCC[C@H](C)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 218

### Prophetic Synthesis of Multimeric Compound 281

Compound 281: Compound 281 can be prepared in an analogous fashion to FIG. 26 using propargyl ether in place of compound 36 in step c.

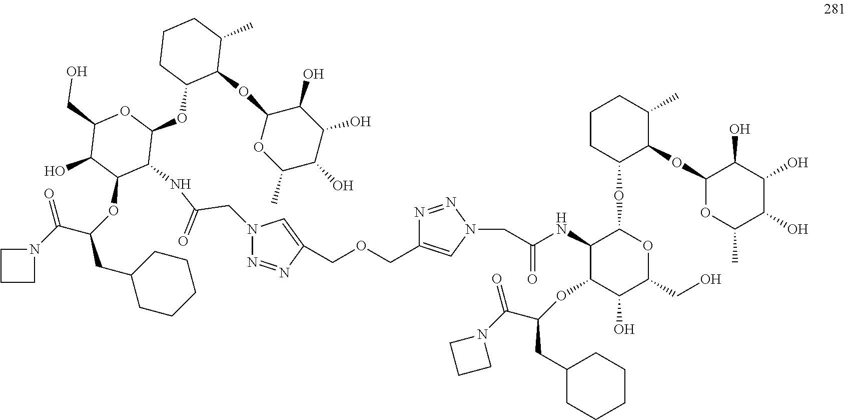 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)N3CCC3)[C@H]2NC(=O)Cn2cc(COCc3cn(CC(=O)N[C@H]4[C@H](O[C@@H]5CCC[C@H](C)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)N4CCC4)nn3)nn2)[C@@H](O)[C@H](O)[C@@H]1O]

### Example 219

### Prophetic Synthesis of Multimeric Compound 282

Compound 282: Compound 282 can be prepared in an analogous fashion to FIG. 26 by replacing compound 43 with ethylene glycol bis propargyl ether in step c.

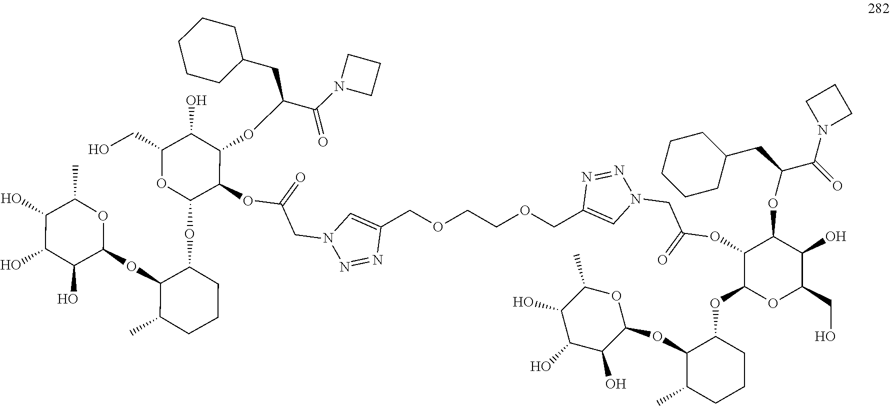 [SMILES: C[C@@H]1O[C@@H](O[C@@H]2[C@@H](C)CCC[C@H]2O[C@@H]2O[C@H](CO)[C@H](O)[C@H](O[C@@H](CC3CCCCC3)C(=O)N3CCC3)[C@H]2OC(=O)Cn2cc(COCCOCc3cn(CC(=O)O[C@H]4[C@H](O[C@@H]5CCC[C@H](C)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)N4CCC4)nn3)nn2)[C@@H](O)[C@H](O)[C@@H]1O]

### Example 220

### Prophetic Synthesis of Multimeric Compound 294

Compound 284: A mixture of compounds 283 (preparation described in WO 2007/028050) and compound 2 (preparation described in WO 2013/096926) (1.7 eq) is azeotroped 3 times from toluene. The mixture is dissolved in DCM under argon and cooled on an ice bath. To this solution is added boron trifluoride etherate (1.5 eq). The reaction mixture is stirred 12 hours at room temperature. The reaction is quenched by the addition of triethylamine (2 eq). The reaction mixture is transferred to a separatory funnel and washed 1 time with half saturated sodium bicarbonate solution and 1 time with water. The organic phase is dried over sodium sulfate, filtered, and concentrated. The residue is purified by flash chromatography to afford compound 284.

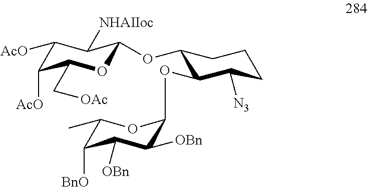

Compound 285: Compound 284 is dissolved in methanol at room temperature. A solution of sodium methoxide in methanol (0.1 eq) is added and the reaction mixture stirred overnight at room temperature. The reaction mixture is quenched by the addition of acetic acid. The reaction mixture is diluted with ethyl acetate, transferred to a separatory funnel and washed 2 times with water. The organic phase is dried over magnesium sulfate, filtered and concentrated. The residue is separated by flash chromatography to afford compound 285.

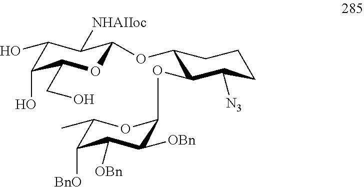

Compound 286: To a solution of compound 285 in dichloromethane cooled on an ice bath is added DABCO (1.5 eq) followed by monomethyoxytrityl chloride (1.2 eq). The reaction mixture is stirred overnight allowing to warm to room temperature. The reaction mixture is transferred to a separatory funnel and washed 2 times with water. The organic phase is concentrated and the residue is purified by flash chromatography to afford compound 286.

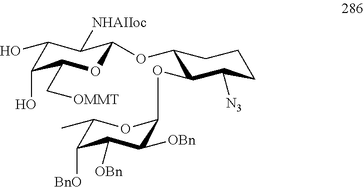

Compound 287: To a solution of compound 286 in methanol is added dibutyltin oxide (1.1 eq). The reaction mixture is refluxed for 3 hours then concentrated. The residue is suspended in DME. To this suspension is added compound 6 (preparation described in Thoma et. al. J. Med. Chem., 1999, 42, 4909) (1.5 eq) followed by cesium fluoride (1.2 eq). The reaction mixture is stirred at room temperature overnight. The reaction mixture is diluted with ethyl acetate, transferred to a separatory funnel, and washed with water. The organic phase is dried over sodium sulfate, filtered and concentrated. The residue is purified by flash chromatography to afford compound 287.

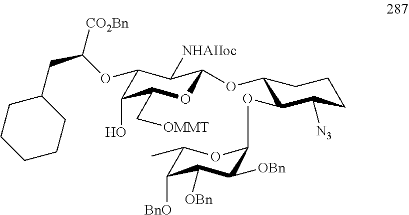

Compound 288: To a degassed solution of compound 287 in anhydrous DCM at 0° C. is added Pd(PPh₃)₄ (0.1 eq), Bu₃SnH (1.1 eq) and N-trifluoroacetyl glycine anhydride (2.0 eq) (preparation described in Chemische Berichte (1955), 88(1), 26). The resulting solution is stirred for 12 hrs allowing the temperature to increase to room temperature. The reaction mixture is diluted with DCM, transferred to a separatory funnel, and washed with water. The organic phase is dried over Na₂SO₄, then filtered and concentrated. The residue is purified by flash chromatography to afford compound 288.

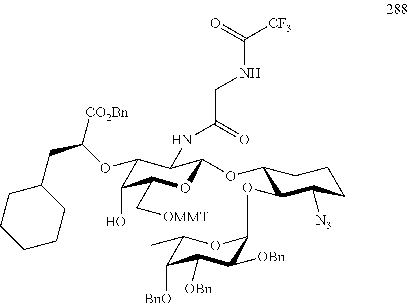

Compound 289: To a stirred solution of compound 288 in DCM/MeOH (25/1) at room temperature is added orotic acid chloride (5 eq) and triphenylphosphine (5 eq). The reaction mixture is stirred 24 hours. The solvent is removed and the residue is separated by column chromatography to afford compound 289.

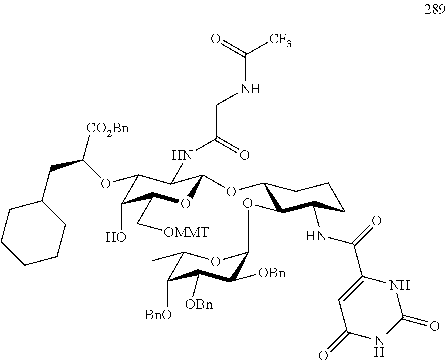

Compound 290: Compound 289 is dissolved in methanol and degassed. To this solution is added Pd(OH)₂/C. The reaction mixture is vigorously stirred under a hydrogen atmosphere for 12 hours. The reaction mixture is filtered through a Celite pad. The filtrate is concentrated under reduced pressure to give compound 290.

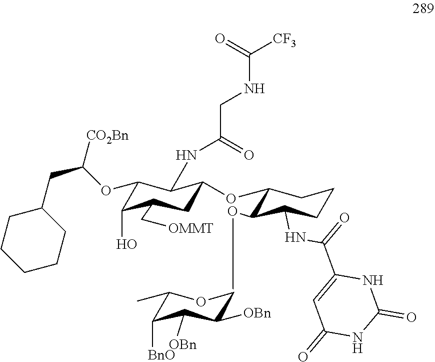

Compound 291: Compound 290 is dissolved in methanol at room temperature. A solution of sodium methoxide in methanol (1.1 eq) is added and the reaction mixture stirred overnight at room temperature. The reaction mixture is quenched by the addition of acetic acid. The reaction mixture is concentrated. The residue is separated by C-18 reverse phase chromatography to afford compound 291.

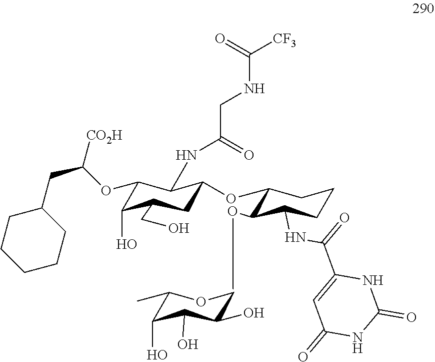

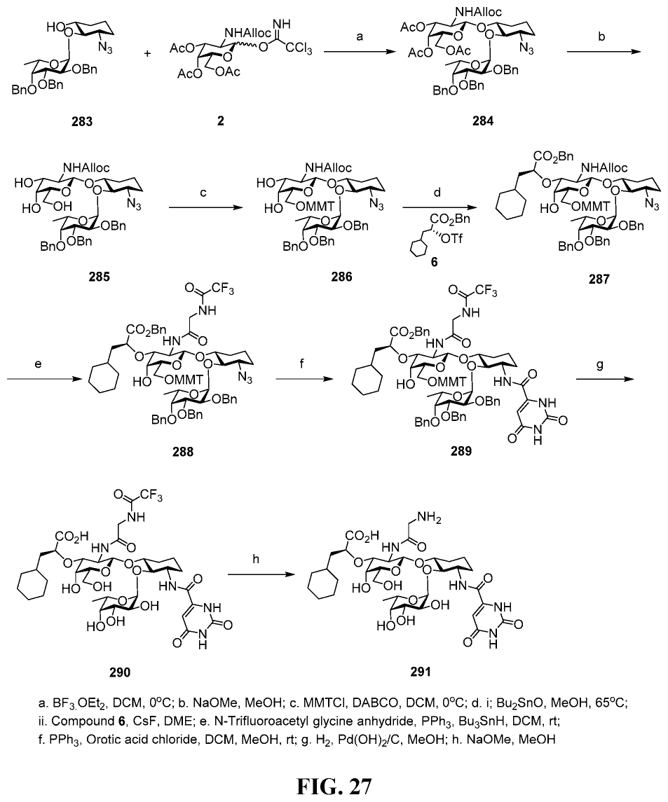

Compound 292: Compound 292 can be prepared in an analogous fashion to FIG. 27 by replacing orotic acid chloride with acetyl chloride in step f.

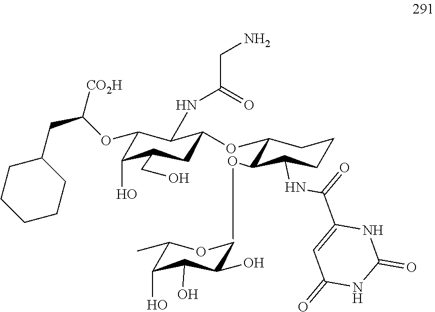

Compound 293: Compound 293 can be prepared in an analogous fashion to FIG. 27 by replacing orotic acid chloride with benzoyl chloride in step f.

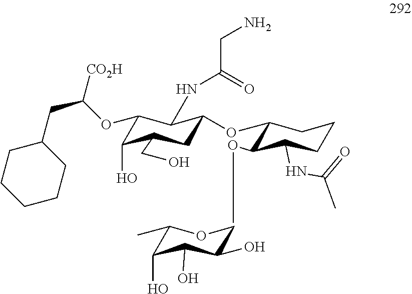

Compound 294: A solution of compound 291 (0.4 eq) in DMSO is added to a solution of compound 20 (1 eq) and DIPEA (10 eq) in anhydrous DMSO at room temperature. The resulting solution is stirred overnight. The reaction mixture is separated by reverse phase chromatography and the product lyophilized to give compound 294.

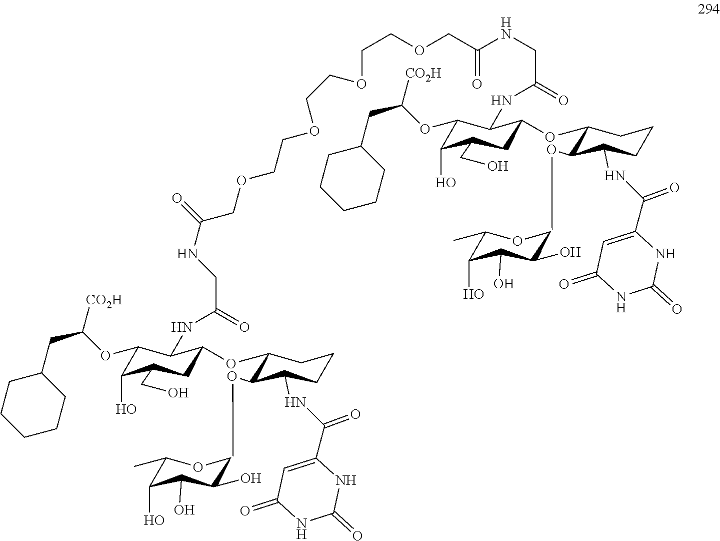

### Example 221

### Prophetic Synthesis of Multimeric Compound 295

Compound 295: Compound 294 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by reverse phase chromatography to afford compound 295.

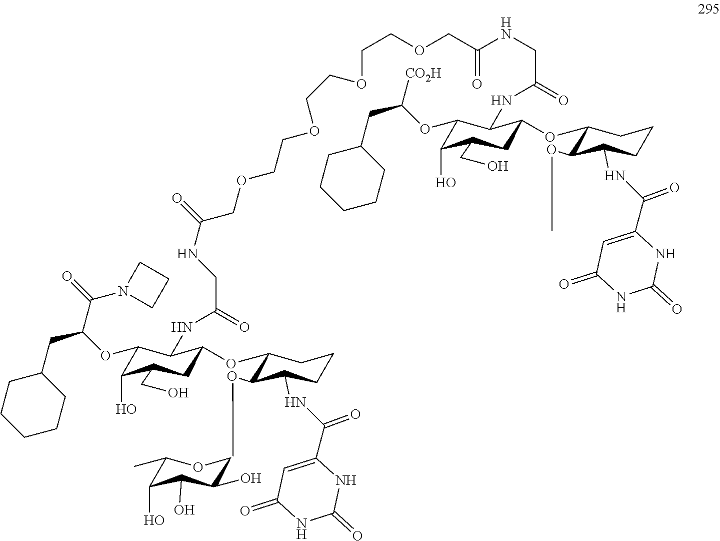

### Example 222

### Prophetic Synthesis of Multimeric Compound 296

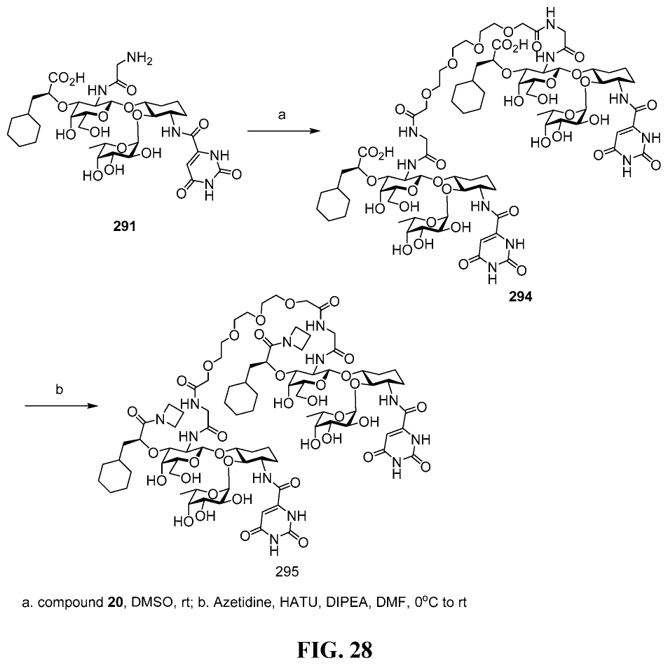

Compound 296: Compound 296 can be prepared in an analogous fashion to FIG. 28 by replacing compound 20 with ethylene glycol diacetic acid di-NHS ester in step a.

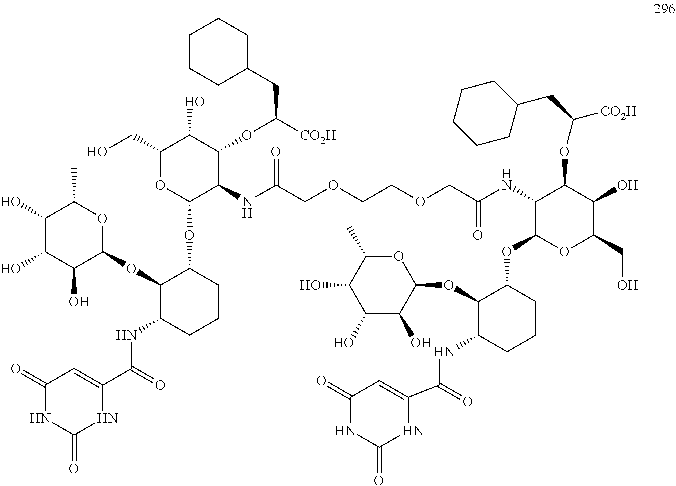 [SMILES: C[C@@H]1O[C@@H](O[C@@H]2[C@@H](NC(=O)c3cc(=O)[nH]c(=O)[nH]3)CCC[C@H]2O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3CCC[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H](O)[C@H](O)[C@@H]1O]

### Example 223

### Prophetic Synthesis of Multimeric Compound 297

Compound 297: Compound 297 can be prepared in an analogous fashion to FIG. 28 by replacing compound 20 with ethylene glycol diacetic acid di-NHS ester in step 00

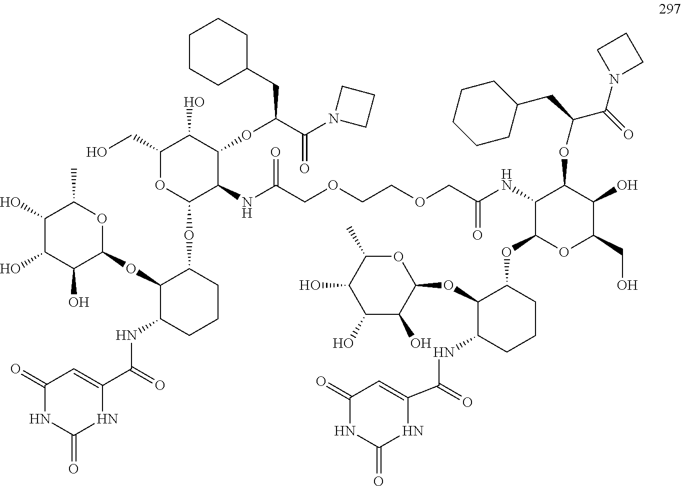 [SMILES: C[C@@H]1O[C@@H](O[C@@H]2[C@@H](NC(=O)c3cc(=O)[nH]c(=O)[nH]3)CCC[C@H]2O[C@@H]2O[C@H](CO)[C@H](O)[C@H](O[C@@H](CC3CCCCC3)C(=O)N3CCC3)[C@H]2NC(=O)COCCOCC(=O)N[C@H]2[C@H](O[C@@H]3CCC[C@H](NC(=O)c4cc(=O)[nH]c(=O)[nH]4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCC2)[C@@H](O)[C@H](O)[C@@H]1O]

### Example 224

### Prophetic Synthesis of Multimeric Compound 298

Compound 298: Compound 298 can be prepared in an analogous fashion to FIG. 28 by replacing compound 291 with compound 292 and compound 20 with ethylene glycol diacetic acid di-NHS ester in step a.

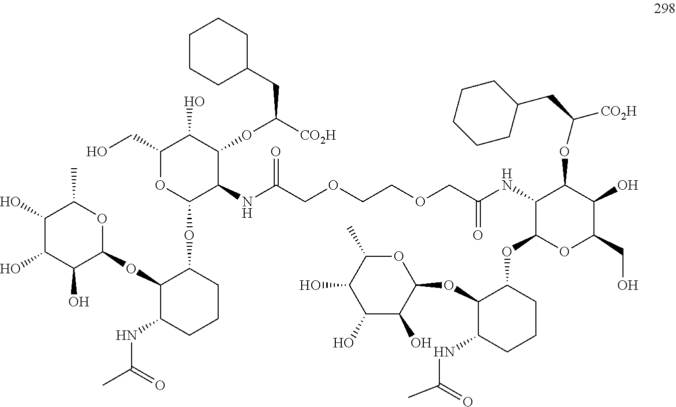 [SMILES: CC(=O)N[C@H]1CCC[C@@H](O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3CCC[C@H](NC(C)=O)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H]1O[C@@H]1O[C@@H](C)[C@@H](O)[C@@H](O)[C@@H]1O]

### Example 225

### Prophetic Synthesis of Multimeric Compound 299

Compound 299: Compound 299 can be prepared in an analogous fashion to FIG. 28 by replacing compound 291 with compound 292 and compound 20 with ethylene glycol diacetic acid di-NHS ester in step a.

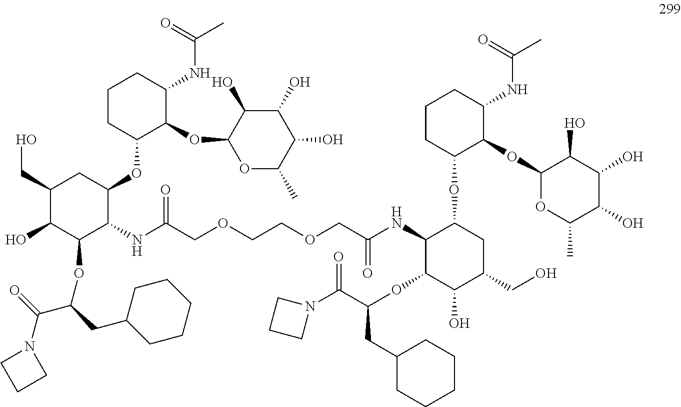 [SMILES: CC(=O)N[C@H]1CCC[C@@H](O[C@@H]2C[C@H](CO)[C@H](O)[C@H](O[C@@H](CC3CCCCC3)C(=O)N3CCC3)[C@H]2NC(=O)COCCOCC(=O)N[C@@H]2[C@@H](O[C@@H](CC3CCCCC3)C(=O)N3CCC3)[C@@H](O)[C@@H](CO)C[C@H]2O[C@@H]2CCC[C@H](NC(C)=O)[C@H]2O[C@@H]2O[C@@H](C)[C@@H](O)[C@@H](O)[C@@H]2O)[C@@H]1O[C@@H]1O[C@@H](C)[C@@H](O)[C@@H](O)[C@@H]1O]

### Example 226

### Prophetic Synthesis of Multimeric Compound 300

Compound 300: Compound 300 can be prepared in an analogous fashion to FIG. 28 by replacing compound 291 with compound 293 and compound 20 with ethylene glycol diacetic acid di-NHS ester in step a.

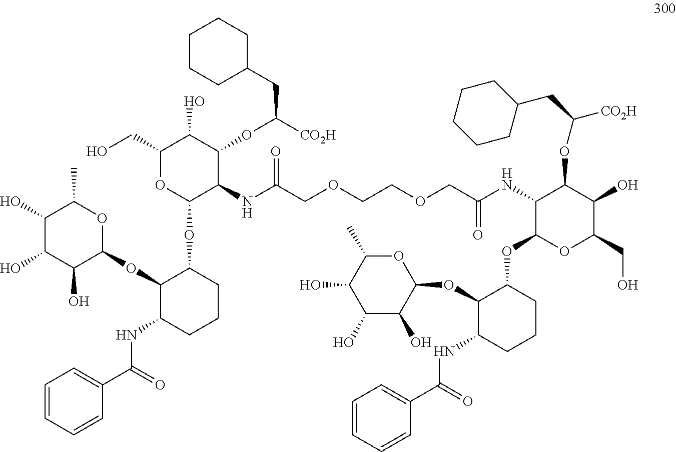 [SMILES: C[C@@H]1O[C@@H](O[C@@H]2[C@@H](NC(=O)c3ccccc3)CCC[C@H]2O[C@@H]2O[C@H](CO)[C@H](O)[C@H](O[C@@H](CC3CCCCC3)C(=O)O)[C@H]2NC(=O)COCCOCC(=O)N[C@H]2[C@H](O[C@@H]3CCC[C@H](NC(=O)c4ccccc4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)O)[C@@H](O)[C@H](O)[C@@H]1O]

### Example 227

### Prophetic Synthesis of Multimeric Compound 301

Compound 301: Compound 301 can be prepared in an analogous fashion to FIG. 28 by replacing compound 291 with compound 293 and compound 20 with ethylene glycol diacetic acid di-NHS ester in step a.

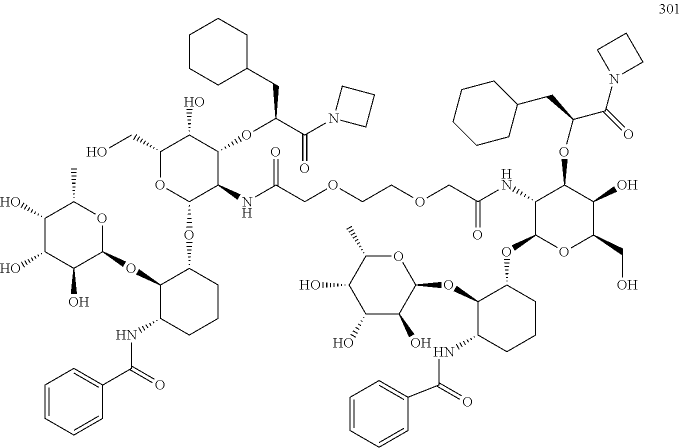 [SMILES: C[C@@H]1O[C@@H](O[C@@H]2[C@@H](NC(=O)c3ccccc3)CCC[C@H]2O[C@@H]2O[C@H](CO)[C@H](O)[C@H](O[C@@H](CC3CCCCC3)C(=O)N3CCC3)[C@H]2NC(=O)COCCOCC(=O)N[C@H]2[C@H](O[C@@H]3CCC[C@H](NC(=O)c4ccccc4)[C@H]3O[C@@H]3O[C@@H](C)[C@@H](O)[C@@H](O)[C@@H]3O)O[C@H](CO)[C@H](O)[C@@H]2O[C@@H](CC2CCCCC2)C(=O)N2CCC2)[C@@H](O)[C@H](O)[C@@H]1O]

### Example 228

### Prophetic Synthesis of Multimeric Compound 302

Compound 302: Compound 302 can be prepared in an analogous fashion to FIG. 28 by replacing compound 20 with 3,3′-[[2,2-bis[[3-[(2,5-dioxo-1-pyrrolidinyl)oxy]-3-oxopropoxy]methyl]-1,3-propanediyl]bis(oxy)]bis-1,1′-bis(2,5-dioxo-1-pyrrolidinyl)-propanoic acid ester in step a.

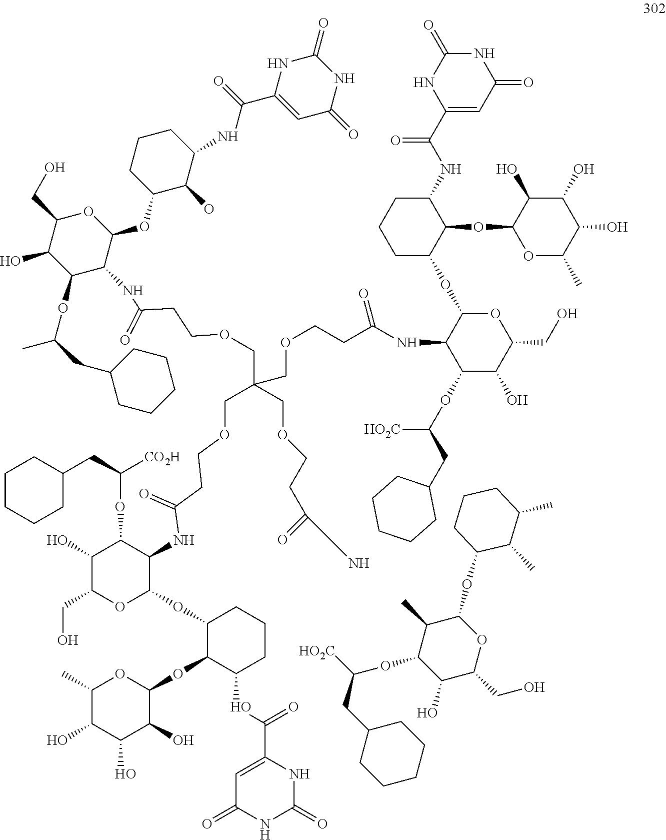

### Example 229

### Prophetic Synthesis of Multimeric Compound 305

Compound 303: To a stirred solution of compound 287 in DCM/MeOH (25/1) at room temperature is added orotic acid chloride (5 eq) and triphenylphosphine (5 eq). The reaction mixture is stirred 24 hours. The solvent is removed and the residue is separated by column chromatography to afford compound 303.

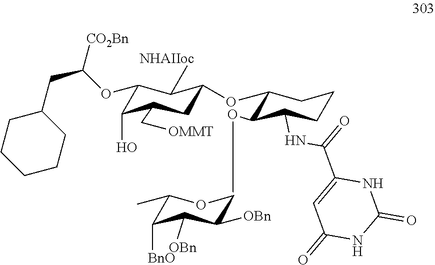

Compound 304: To a degassed solution of compound 303 in anhydrous DCM at 0° C. is added Pd(PPh₃)₄ (0.1 eq), Bu₃SnH (1.1 eq) and azidoacetic anhydride (2.0 eq). The ice bath is removed and the solution is stirred for 12 hrs under a N₂ atmosphere at room temperature. The reaction mixture is diluted with DCM, washed with water, dried over Na₂SO₄, then concentrated. The crude product is purified by column chromatography to give compound 304.

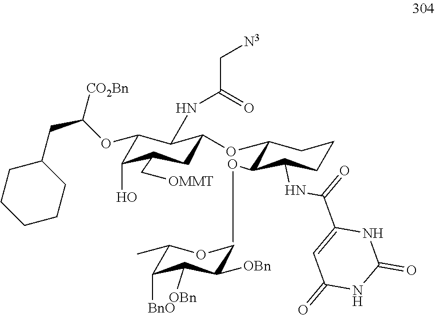

Compound 305: A solution of bispropagyl PEG-5 (compound 43) and compound 304 (2.4 eq) in MeOH is degassed at room temperature. A solution of CuSO₄/THPTA in distilled water (0.04 M) (0.2 eq) and sodium ascorbate (0.2 eq) are added successively and the resulting solution is stirred 12 hrs at 50° C. The solution is cooled to room temperature and concentrated under reduced pressure. The crude product is purified by chromatography to give compound 305.

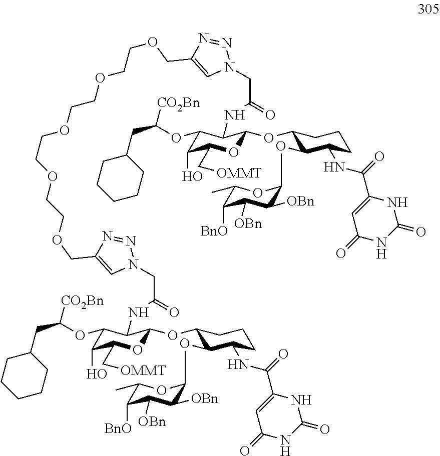

### Example 230

### Prophetic Synthesis of Multimeric Compound 306

Compound 306: Compound 305 is dissolved in MeOH and hydrogenated in the presence of Pd(OH)₂ (20 wt %) at 1 atm of H₂ gas pressure for 24 hrs at room temperature. The solution is filtered through a Celite pad. The filtrate is concentrated to give compound 306.

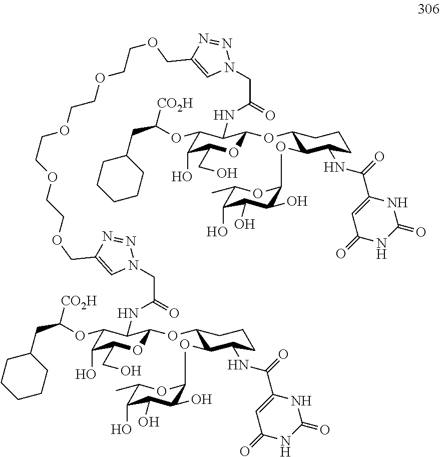

### Example 231

### Prophetic Synthesis of Multimeric Compound 307

Compound 307: Compound 306 is dissolved in DMF and cooled on an ice bath. Diisopropylethylamine (2.5 eq) is added followed by HATU (2.2 eq). The reaction mixture is stirred 15 minutes on the ice bath then azetidine (10 eq) is added. The ice bath is removed and the reaction mixture is stirred overnight at room temperature. The solvent is removed under reduced pressure and the residue is separated by reverse phase chromatography to afford compound 307.

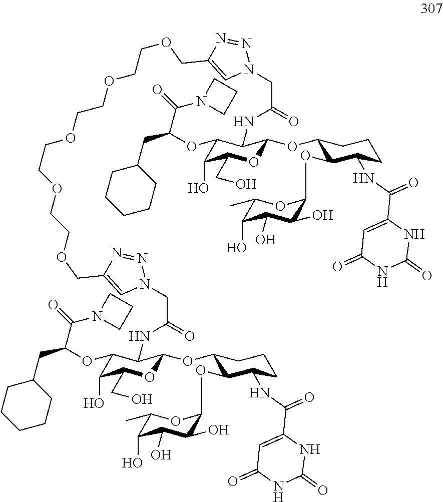

### Example 232

### Prophetic Synthesis of Multimeric Compound 308

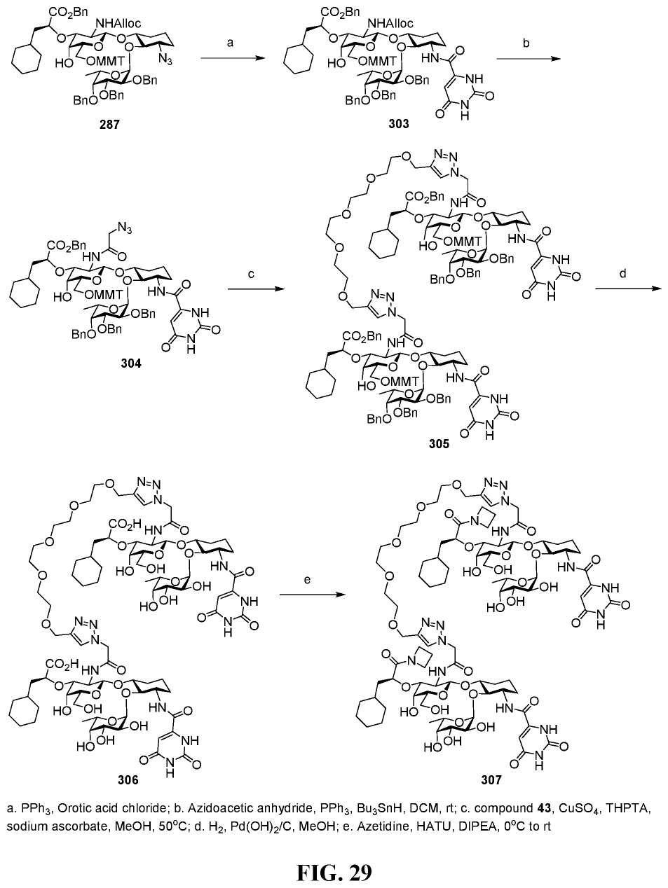

Compound 308: Compound 308 can be prepared in an analogous fashion to FIG. 29 using 3,3′-[[2,2-bis[(2-propyn-1-yloxy)methyl]-1,3-propanediyl]bis(oxy)]bis-1-propyne in place of compound 43 in step c.

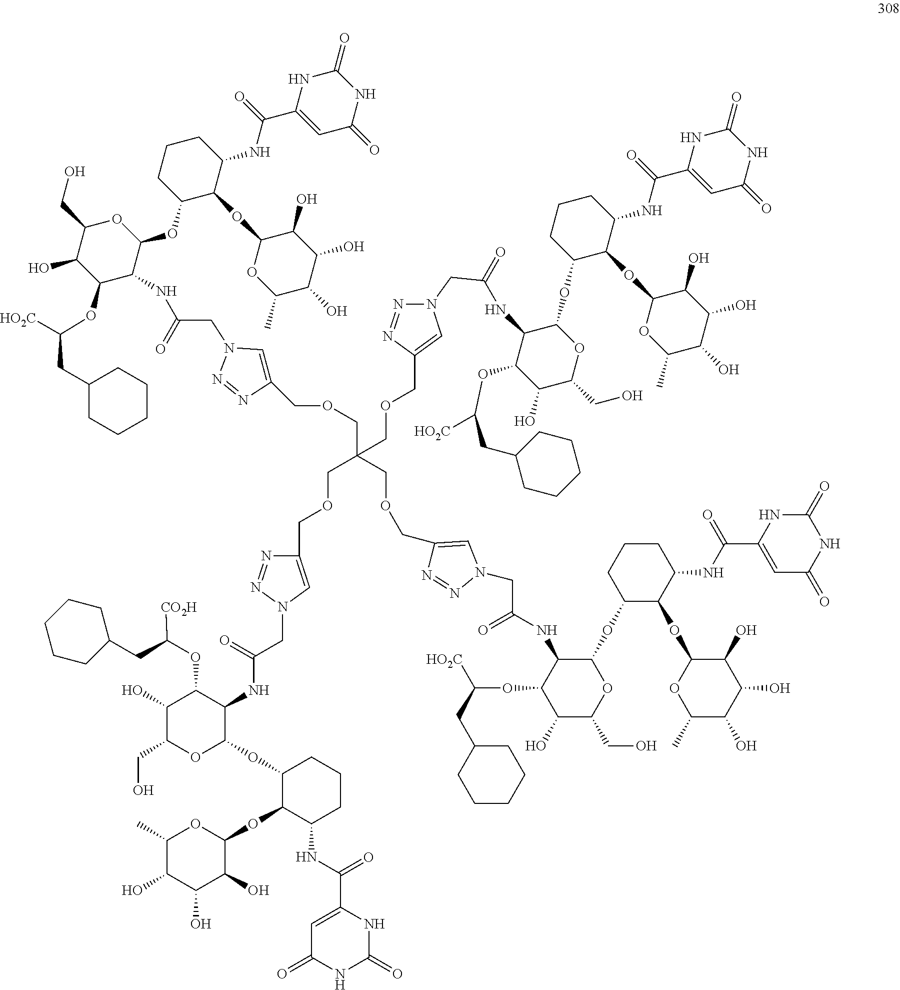

### Example 233

### Prophetic Synthesis of Multimeric Compound 309

Compound 309: Compound 309 can be prepared in an analogous fashion to FIG. 29 using 3,3′-[[2,2-bis[(2-propyn-1-yloxy)methyl]-1,3-propanediyl]bis(oxy)]bis-1-propyne in place of compound 43 in step c.

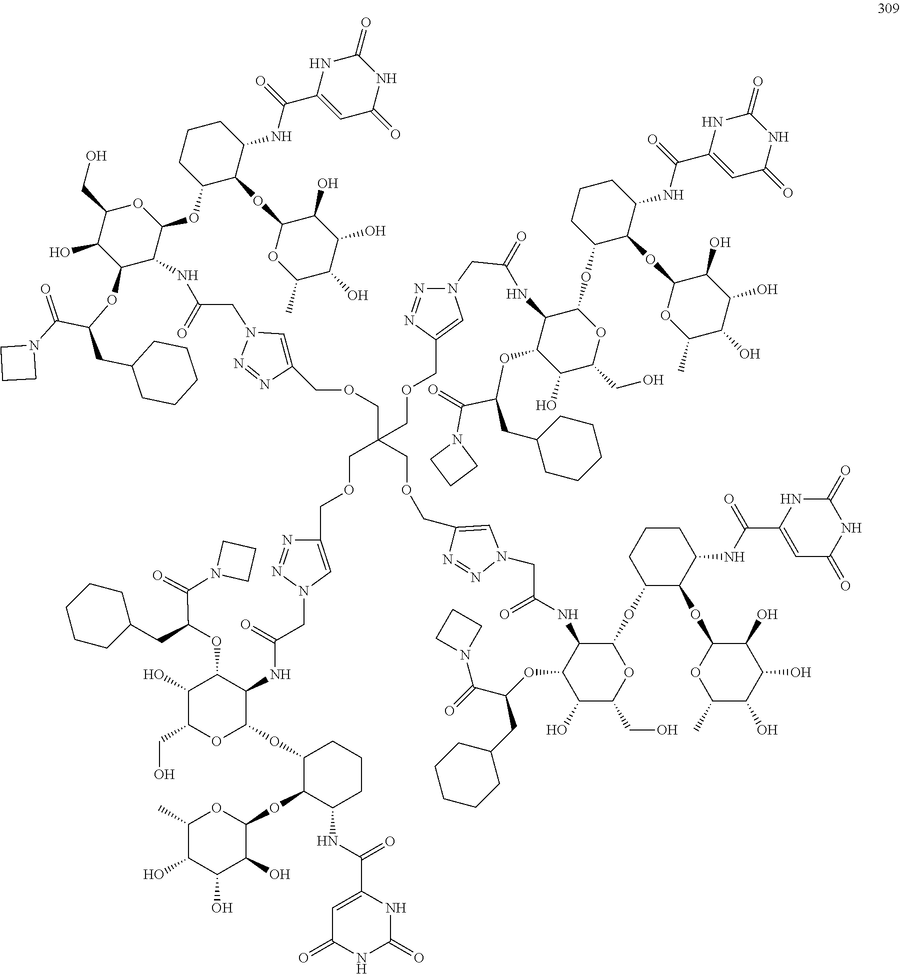

### Example 234

### Prophetic Synthesis of Multimeric Compound 310

Compound 310: Compound 310 can be prepared in an analogous fashion to FIG. 29 by replacing compound 43 with bis-propargyl ethylene glycol in step c.

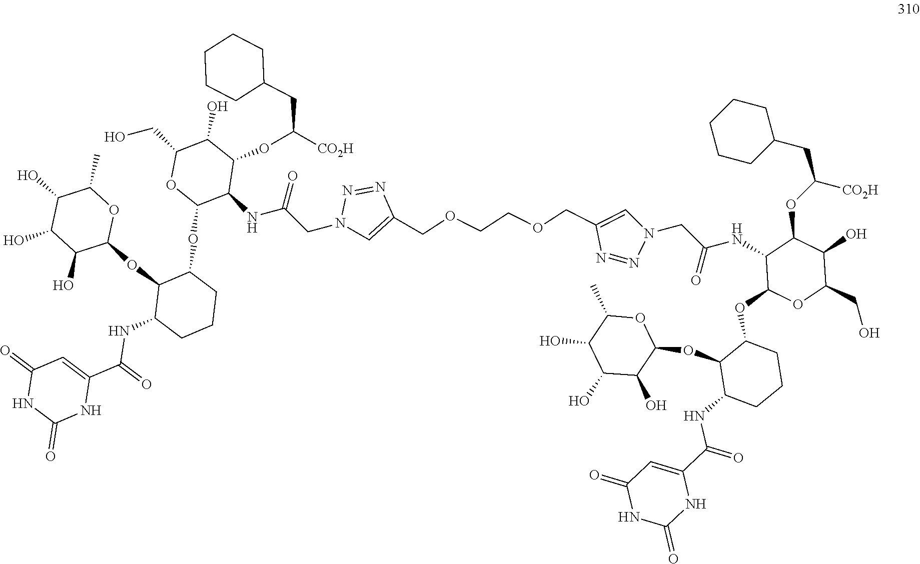 [SMILES: C[C@@H]1O[C@@H](O[C@@H]2[C@@H](NC(=O)c3cc(=O)[nH]c(=O)[nH]3)CCC[C@H]2O[C@@H]2O[C@H](CO)[C@H](O)[C@H](O[C@@H](CC3CCCCC3)C(=O)O)[C@H]2NC(=O)Cn2cc(COCCOCc3cn(CC(=O)N[C@H]4[C@H](O[C@@H]5CCC[C@H](NC(=O)c6cc(=O)[nH]c(=O)[nH]6)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 235

### Prophetic Synthesis of Multimeric Compound 311

Compound 311: Compound 311 can be prepared in an analogous fashion to FIG. 29 by replacing compound 43 with bis-propargyl ethylene glycol in step c.

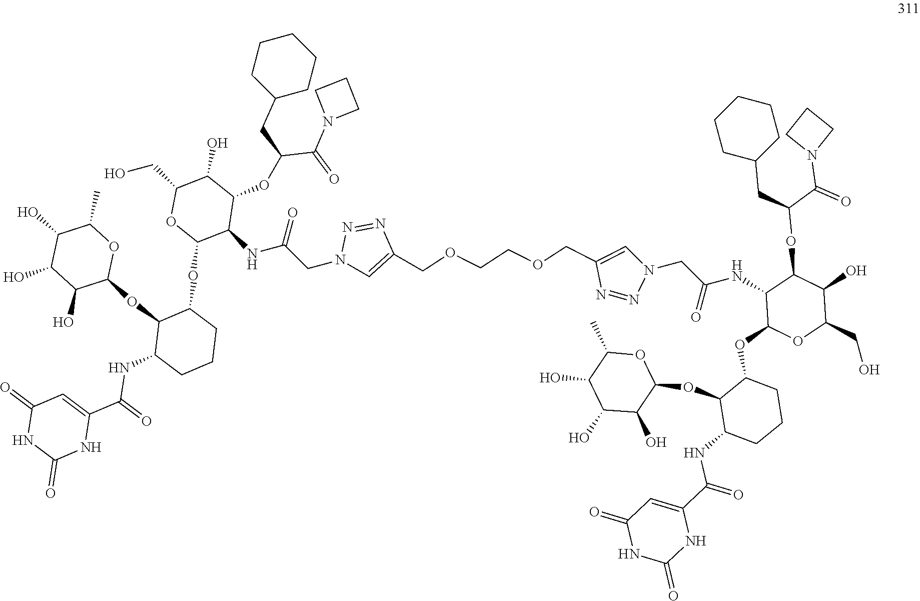 [SMILES: C[C@@H]1O[C@@H](O[C@@H]2[C@@H](NC(=O)c3cc(=O)[nH]c(=O)[nH]3)CCC[C@H]2O[C@@H]2O[C@H](CO)[C@H](O)[C@H](O[C@@H](CC3CCCCC3)C(=O)N3CCC3)[C@H]2NC(=O)Cn2cc(COCCOCc3cn(CC(=O)N[C@H]4[C@H](O[C@@H]5CCC[C@H](NC(=O)c6cc(=O)[nH]c(=O)[nH]6)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)N4CCC4)nn3)nn2)[C@@H](O)[C@H](O)[C@@H]1O]

### Example 236

### Prophetic Synthesis of Multimeric Compound 312

Compound 312: Compound 312 can be prepared in an analogous fashion to FIG. 29 by replacing compound 43 with propargyl ether in step c.

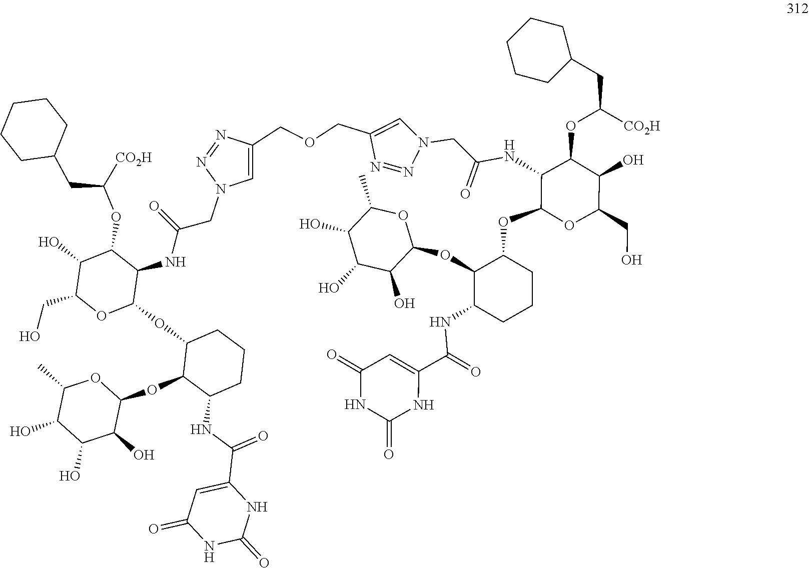 [SMILES: C[C@@H]1O[C@@H](O[C@@H]2[C@@H](NC(=O)c3cc(=O)[nH]c(=O)[nH]3)CCC[C@H]2O[C@@H]2O[C@H](CO)[C@H](O)[C@H](O[C@@H](CC3CCCCC3)C(=O)O)[C@H]2NC(=O)Cn2cc(COCc3cn(CC(=O)N[C@H]4[C@H](O[C@@H]5CCC[C@H](NC(=O)c6cc(=O)[nH]c(=O)[nH]6)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)O)nn3)nn2)[C@@H](O)[C@H](O)[C@@H]1O]

### Example 237

### Prophetic Synthesis of Multimeric Compound 313

Compound 313: Compound 313 can be prepared in an analogous fashion to FIG. 29 by replacing compound 43 with propargyl ether in step c.

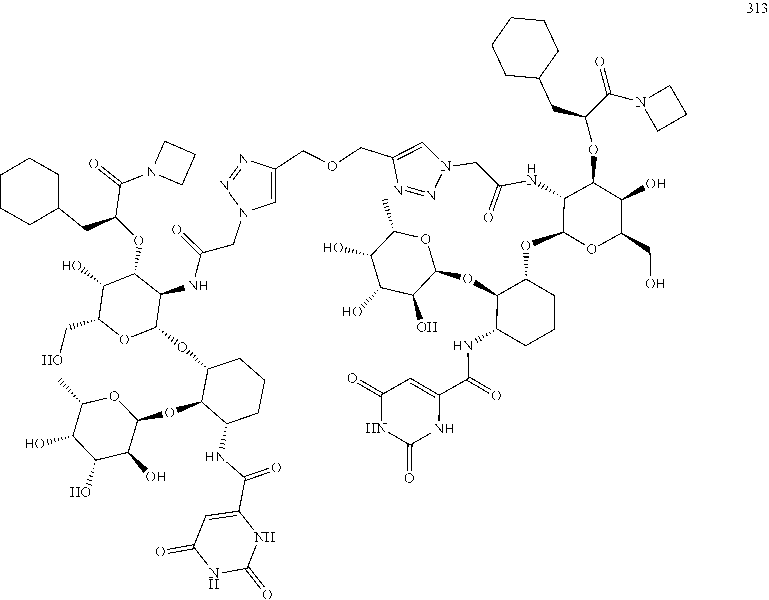 [SMILES: C[C@@H]1O[C@@H](O[C@@H]2[C@@H](NC(=O)c3cc(=O)[nH]c(=O)[nH]3)CCC[C@H]2O[C@@H]2O[C@H](CO)[C@H](O)[C@H](O[C@@H](CC3CCCCC3)C(=O)N3CCC3)[C@H]2NC(=O)Cn2cc(COCc3cn(CC(=O)N[C@H]4[C@H](O[C@@H]5CCC[C@H](NC(=O)c6cc(=O)[nH]c(=O)[nH]6)[C@H]5O[C@@H]5O[C@@H](C)[C@@H](O)[C@@H](O)[C@@H]5O)O[C@H](CO)[C@H](O)[C@@H]4O[C@@H](CC4CCCCC4)C(=O)N4CCC4)nn3)nn2)[C@@H](O)[C@H](O)[C@@H]1O]

### Example 238

### Synthesis of Building Block 332

Compound 321: Compound 317 (1.1 g, 2.60 mmoles) was dissolved in methanol (25 mL) at room temperature, Sodium methoxide (0.1 mL, 25% sol. in MeOH) was added and the reaction mixture was stirred at room temperature for 2 hours. The reaction mixture neutralized by the addition of Amberlyst acidic resin, filtered and concentrated to give crude 321, which was used for the next step without further purification. LCMS (ESI): m %-calculated for C₁₂H₁₅N₃O₄S: 297.3, found 298.1 (M+1), 320.1 (M+Na).

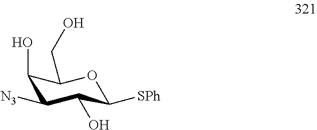

Compound 322: Crude compound 321 (2.60 mmoles), 3,4,5-trifluorophenyl-1-acetylene (2.5 equiv), THPTA (0.11 equiv), and copper (II) sulfate (0.1) were dissolved in methanol (15 mL) at room temperature. Sodium ascorbate (2.4 equiv) dissolved in water was added and the reaction mixture was stirred overnight at room temperature. The resultant precipitate was collected by filtration, washed with hexanes and water, and dried to give compound 322 as a pale yellow solid (1.2 g, 100% yield for 2 steps). LCMS (ESI): m/z calculated for C₂₀H₁₈F₃N₃O₄S: 453.1, found 454.2 (M+1); 476.2 (M+Na).

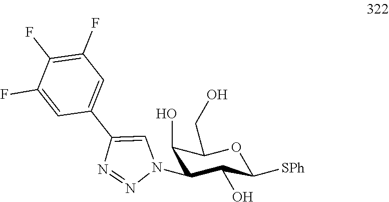

Compound 323: Compound 322 (1.2 g, 2.65 mmoles) was dissolved in DMF (15 mL) and cooled on an ice bath. Sodium hydride (60/o oil dispersion, 477 mg, 11.93 mmoles) was added and the mixture stirred for 30 minutes. Benzyl bromide (1.42 mL, 11.93 mmoles) was added and the reaction was warmed to room temperature and stirred overnight. The reaction mixture was quenched by the addition of aqueous saturated ammonium chloride solution, transferred to a separatory funnel and extracted 3 times with ether. The combined organic phases were dried over magnesium sulfate, filtered, and concentrated. The residue was purified by flash chromatography to afford compound 323 (1.8 g, 94% yield). LCMS (ESI): m/z calculated for C₄₁H₃₆F₃N₃O₄S: 723.2, found 724.3 (M+1); 746.3 (M+Na).

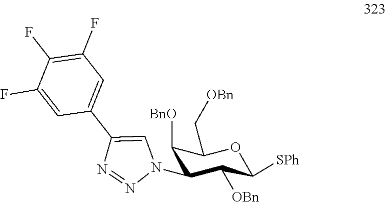

Compound 324: Compound 323 (1.8 g, 2.49 mmol) was dissolved in acetone (20 mL) and water (2 mL) and cooled on an ice bath. Trichloroisocyanuric acid (637 mg, 2.74 mmoles) was added and the reaction mixture stirred on the ice bath for 3 h. The acetone was removed in vacuo and the residue was diluted with DCM, transferred to a separatory funnel, and washed with saturated aqueous NaHCO₃. The organic phase was concentrated and the residue was purified by flash chromatography to afford compound 324 (1.5 g, 95%). LCMS (ESI): m/z calculated for C₃₅H₃₂F₃N₃O₅: 631.2, found 632.2 (M+1); 654.2 (M+Na).

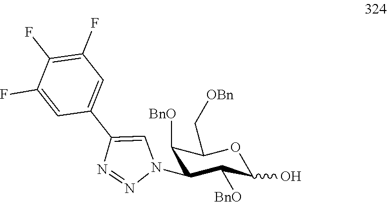

Compound 325: Compound 324 (1.0 g, 1.58 mmoles) was dissolved in DCM (20 mL) and cooled on an ice bath. Dess-Martin periodinane (1.0 g, 2.37 mmoles) was added and mixture was allowed to warm to room temperature and stirred overnight. The reaction mixture quenched by the addition of aqueous saturated NaHCO₃, transferred to a separatory funnel, and extracted 2 times with DCM. The combined organic phases were dried over sodium sulfate, filtered, and concentrated. The residue was purified by flash chromatography to afford compound 325 (520 mg, 52% yield). LCMS (ESI): m/z calculated for C₃₅H₃₀F₃N₃O₅: 629.2, found 652.2 (M+Na); 662.2 (M+MeOH+1); 684.2 (M+MeOH+Na).

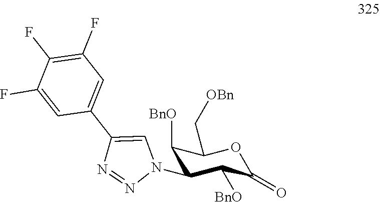

Compound 326: Methyl bromoacetate (253 mg, 1.65 mmoles) dissolved in 0.5 mL of THF was added dropwise to a solution of lithium bis(trimethylsilyl)amide (1.0 M in THF, 1.65 mL, 1.65 mmoles) cooled at −78 C. The reaction mixture was stirred for 30 minutes at −78 C. Compound 325 (260 mg, 0.41 mmoles) dissolved in THF (2.0 mL) was then added. The reaction mixture was stirred at −78 C for 30 minutes. The reaction was quenched by the addition of aqueous saturated NH₄Cl and warmed to rt. The reaction mixture was transferred to a separatory funnel and extracted 3 times with ethyl acetate. The combined organic phases were dried over sodium sulfate, filtered and concentrated. The residue was separated by flash chromatography to afford compound 326 (183 mg, 64°/yield).

¹H NMR (400 MHz, Chloroform-d) δ 7.38-7.22 (m, 9H), 7.15-7.11 (m, 3H), 7.09 (dd, J=8.4, 6.6 Hz, 1H), 7.06-7.00 (m, 2H), 6.98-6.93 (m, 2H), 5.11 (dd, J=11.3, 3.2 Hz, 1H), 4.60 (d, J=11.8 Hz, 1H), 4.57-4.49 (m, 2H), 4.49-4.42 (m, 2H), 4.35 (d, J=11.8 Hz, 1H), 4.14 (d, J=3.2 Hz, 1H), 4.05 (s, 1H), 4.02 (d, J=7.0 Hz, 1H), 3.84 (d, J=11.0 Hz, 1H), 3.81 (s, 3H), 3.70 (dd, J=9.5, 7.7 Hz, 1H), 3.62 (dd, J=9.4, 6.0 Hz, 1H). LCMS (ESI): m/z calculated for C₃₈H₃₄F₃N₃O₇: 701.2, found 702.3 (M+1); 724.3 (M+Na).

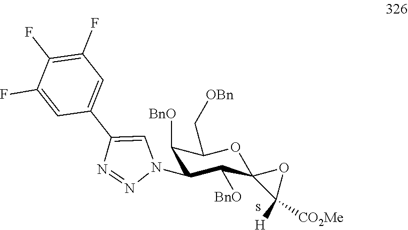

Compound 327: Compound 326 (5.0 g, 7.13 mmol) was azeotroped with toluene two times under reduced pressure, and then dried under high vacuum for 2 hours. It was then dissolved in anhydrous CH₂Cl₂ (125 mL) and cooled on an ice bath while stirring under an atmosphere of argon. Tributyltin hydride (15.1 mL, 56.1 mmol) was added dropwise and the solution was allowed to stir for 25 minutes on the ice bath. Trimethylsilyl triflate (2.1 mL, 11.6 mmol) dissolved in 20 mL of anhydrous CH₂Cl₂ was then added dropwise over the course of 5 minutes. The reaction was slowly warmed to ambient temperature and stirred for 16 hours. The reaction mixture was then diluted with CH₂Cl₂ (50 mL), transferred to a separatory funnel, and washed with saturated aqueous NaHCO₃ (50 mL). The aqueous phase was separated and extracted with CH₂Cl₂ (50 mL×2). The combined organic phases were washed with saturated aqueous NaHCO₃ (50 mL), dried over Na₂SO₄, filtered, and concentrated. The residue was purified by flash chromatography (hexanes to 40% EtOAc in hexanes, gradient) to afford compound 327 (2.65 g, 48%).

¹H-NMR (400 MHz, CDCl₃): δ 7.65 (s, 1H), 7.36-7.22 (m, 8H), 7.16-7.06 (m, 7H), 6.96-6.90 (m, 2H), 5.03 (dd, J=10.7, 3.2 Hz, 1H), 4.72 (d, J=2.3 Hz, 1H), 4.51 (dt, J=22.6, 11.4 Hz, 3H), 4.41 (d, J=10.9 Hz, 1H), 4.32 (dd, J=10.7, 9.2 Hz, 1H), 4.07 (d, J=3.1 Hz, 1H), 3.94 (d, J=10.9 Hz, 1H), 3.92-3.84 (m, 3H), 3.78-3.71 (m, 4H), 3.65 (dd, J=9.1, 5.5 Hz, 1H), 0.24 (s, 9H). LCMS (ESI): m/z (M+Na) calculated for C₄₁H₄₄F₃N₃O₇SiNa: 798.87, found 798.2.

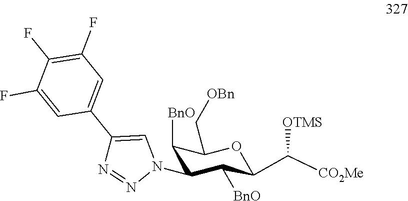

Compound 328: To a solution of compound 327 (2.65 g, 3.4 mmol) in anhydrous MeOH (40 mL) was added Pd(OH)₂ (0.27 g, 20% by wt). The mixture was cooled on an ice bath and stirred for 30 minutes. Triethylsilane (22 mL, 137 mmol) was added dropwise. The solution was allowed to slowly warm to ambient temperature and stirred for 16 hours. The reaction mixture was filtered through a bed of Celite and concentrated. The residue was purified by flash chromatography (hexanes to 100% EtOAc, gradient) to afford compound 328 (1.09 g, 73%).

¹H-NMR (400 MHz, CD₃OD): δ 8.57 (s, 1H), 7.77-7.53 (m, 2H), 4.91-4.82 (m, 1H), 4.66-4.59 (m, 1H), 4.55 (dd, J=10.8, 9.4 Hz, 1H), 4.13 (d, J=2.8 Hz, 1H), 3.86 (dd, J=9.4, 2.1 Hz, 1H), 3.81 (s, 3H), 3.77-3.74 (m, 1H), 3.71-3.68 (m, 2H). LCMS (ESI): m/z (M+Na) calculated for C₁₇H₁₈F₃N₃O₇Na: 456.33, found 456.0.

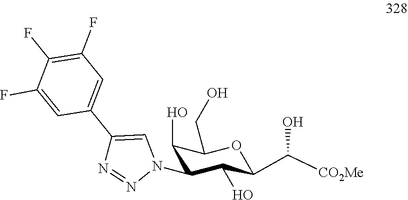

Compound 329: Compound 328 (1.09 g, 2.5 mmol) and CSA (0.115 g, 0.49 mmol) were suspended in anhydrous MeCN (80 mL) under an argon atmosphere. Benzaldehyde dimethyl acetal (0.45 mL, 2.99 mmol) was added dropwise. The reaction mixture was allowed to stir for 16 hours at ambient temperature, during which time it became a homogenous solution. The reaction mixture was then neutralized with a few drops of Et₃N, and concentrated. The residue was purified via flash chromatography (CH₂Cl₂ to 10% MeOH in CH₂Cl₂, gradient) to afford compound 329 (978 mg, 75%).

¹H NMR (400 MHz, DMSO-d6): δ 8.84 (s, 1H), 7.95-7.73 (m, 2H), 7.33 (qdt, J=8.4, 5.6, 2.7 Hz, 5H), 5.51 (t, J=3.8 Hz, 2H), 5.47 (d, J=6.8 Hz, 1H), 5.14 (dd, J=10.8, 3.6 Hz, 1H), 4.54 (dd, J=6.7, 2.2 Hz, 1H), 4.47 (ddd, J=10.8, 9.3, 7.5 Hz, 1H), 4.40 (d, J=4.0 Hz, 1H), 4.09-3.99 (m, 2H), 3.85 (dd, J=9.3, 2.2 Hz, 1H), 3.81-3.76 (m, 1H), 3.71 (s, 3H). LCMS (ESI): m/z (M+Na) calculated for C₂₄H₂₂F₃N₃O₇Na: 544.43, found 544.1.

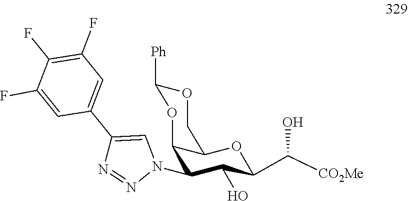

Compound 330: Compound 329 (25.2 mg, 0.048 mmol) was azeotroped with toluene 2 times under reduced pressure, dried under high vacuum for 2 hours, then dissolved in anhydrous DMF (2 mL) and cooled on an ice bath. Benzyl bromide (6 uL, 0.05 mmol) dissolved in 0.5 mL of anhydrous DMF was added and the reaction and was stirred under an atmosphere of argon for 30 minutes at 0° C. Sodium hydride (2 mg, 0.05 mmol, 60%) was added and the reaction was allowed to gradually warm to ambient temperature while stirring for 16 hours. The reaction mixture was diluted with EtOAc (20 mL), transferred to a separatory funnel, and washed with H₂O (10 mL). The aqueous phase was separated and extracted with EtOAc (10 mL×3). The combined organic phases were washed with H₂O (10 mL×3), dried over Na₂SO₄, filtered, and concentrated. The residue was purified via preparative TLC (5% MeOH in CH₂Cl₂) to afford compound 330 (6.3 mg, 21%). LCMS (ESI): m/z (M+Na) calculated for C₃₁H₂₈F₃N₃O₇Na: 634.55, found 634.1.

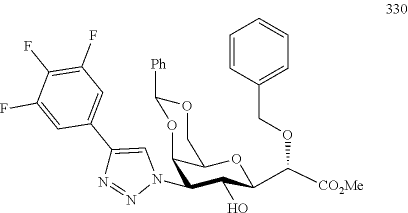

Compound 331: Compound 330 (6.3 mg, 0.01 mmol) was dissolved in anhydrous MeOH (1 mL) containing CSA (0.26 mg, 0.001 mmol). The reaction mixture was heated to 76° C. in a screw-cap scintillation vial while stirring. After 2 hours, an additional 0.13 mg of CSA in 0.5 mL of MeOH was added. The reaction mixture was stirred at 76° C. for 16 hours. The reaction mixture concentrated under reduced pressure. The residue was purified via preparative TLC (10% MeOH in CH₂Cl₂) to afford compound 331 (4.2 mg, 80%).

¹H NMR (400 MHz, DMSO-d₆) δ 8.80 (s, 1H), 7.94-7.86 (m, 2H), 7.48-7.42 (m, 2H), 7.38 (t, J=7.4 Hz, 2H), 7.36-7.28 (m, 1H), 5.46 (d, J=7.7 Hz, 1H), 528 (d, J=6.0 Hz, 1H), 4.85 (dd, J=10.7, 2.9 Hz, 1H), 4.67 (d, J=11.0 Hz, 1H), 4.62-4.58 (m, 1H), 4.54 (d, J=11.1 Hz, 1H), 4.44 (d, J=2.5 Hz, 1H), 4.36 (q, J=9.5 Hz, 1H), 3.95-3.90 (m, 1H), 3.78 (dd, J=9.3, 2.5 Hz, 1H), 3.71 (s, 3H), 3.61-3.54 (m, 1H), 3.52-3.43 (m, 1H), 3.43-3.38 (m, 1H). LCMS (ESI): m/z (M+Na) calculated for C₂₄H₂₄F₃N₃O₇Na: 546,45, found 546.0.

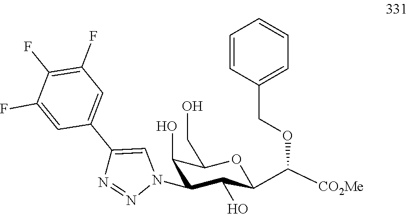

Compound 332: To a solution of compound 331 (3.5 mg, 0.007 mmoles) in methanol (0.5 mL) was added 1.0 M NaOH solution (0.1 mL). The reaction mixture was stirred overnight at room temperature then neutralized with acidic resin, filtered and concentrated. The residue was purified by reverse phase chromatography using a C-8 matrix to afford 3.0 mg compound 332 (90%).

¹H NMR (400 MHz, Deuterium Oxide) δ 8.39 (s, 1H), 8.37 (s, 2H), 7.54-7.45 (m, 1H), 7.43 (d, J=7.4 Hz, 2H), 7.35 (dt, J=14.3, 7.2 Hz, 3H), 4.86 (dd, J=11.0, 2.9 Hz, 1H), 4.76 (d, J=11.0 Hz, 1H), 4.40-4.30 (m, 2H), 4.16 (d, J=1.9 Hz, 1H), 4.04 (d, J=3.0 Hz, 1H), 3.81 (d, J=9.6 Hz, 1H), 3.73 (d, J=3.9 Hz, OH), 3.67 (d, J=7.6 Hz, 1H), 3.56 (dd, J=11.7, 3.9 Hz, 1H). LCMS (ESI): m/z (M+Na) calculated for C₂₃H₂₂F₃N₃O₇: 509.1,

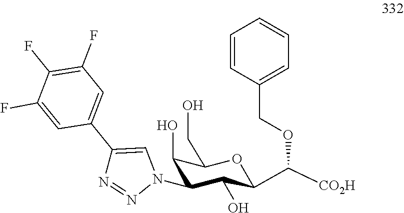

### Example 239

### Prophetic Synthesis of Building Block 333

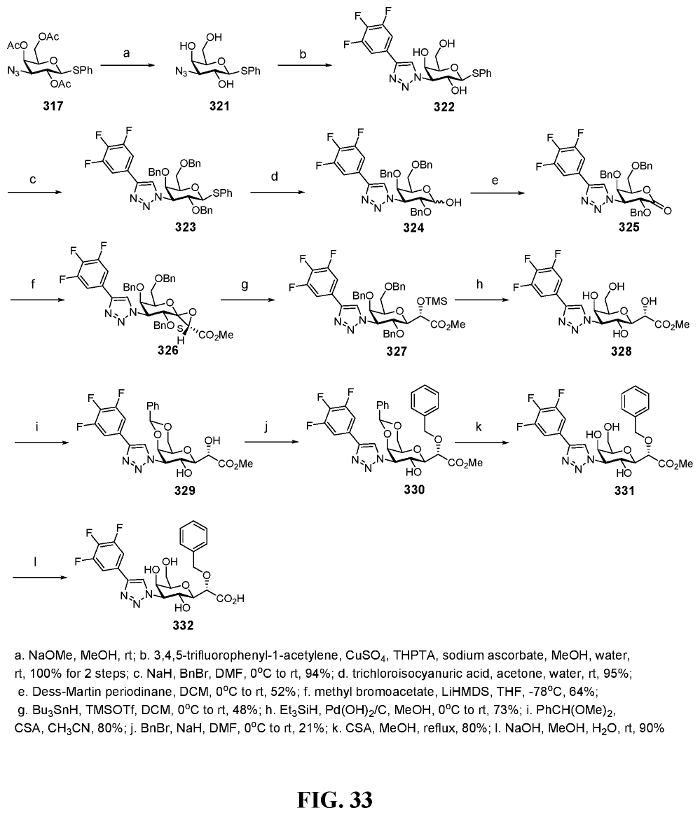

Compound 333: Compound 333 can be prepared in an analogous fashion to FIG. 33 by replacing benzyl bromide with 4-chlorobenzyl bromide in step j.

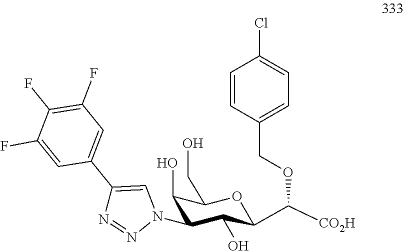

### Example 240

### Prophetic Synthesis of Building Block 334

Compound 334: Compound 334 can be prepared in an analogous fashion to FIG. 33 by replacing benzyl bromide with 4-methanesulfonylbenzoyl bromide in step j.

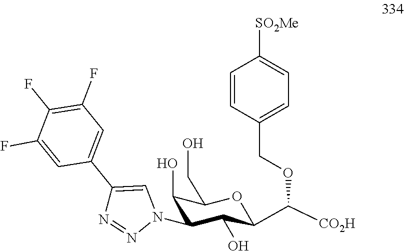

### Example 241

### Prophetic Synthesis of Building Block 335

Compound 335: Compound 335 can be prepared in an analogous fashion to FIG. 33 by replacing benzyl bromide with 3-picolyl bromide in step j.

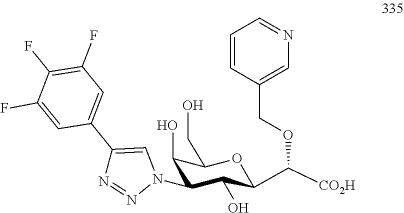

### Example 242

### Prophetic Synthesis of Multimeric Compound 336

Compound 336: Compound 336 can be prepared in an analogous fashion to FIG. 14 by replacing compound 145 with compound 332.

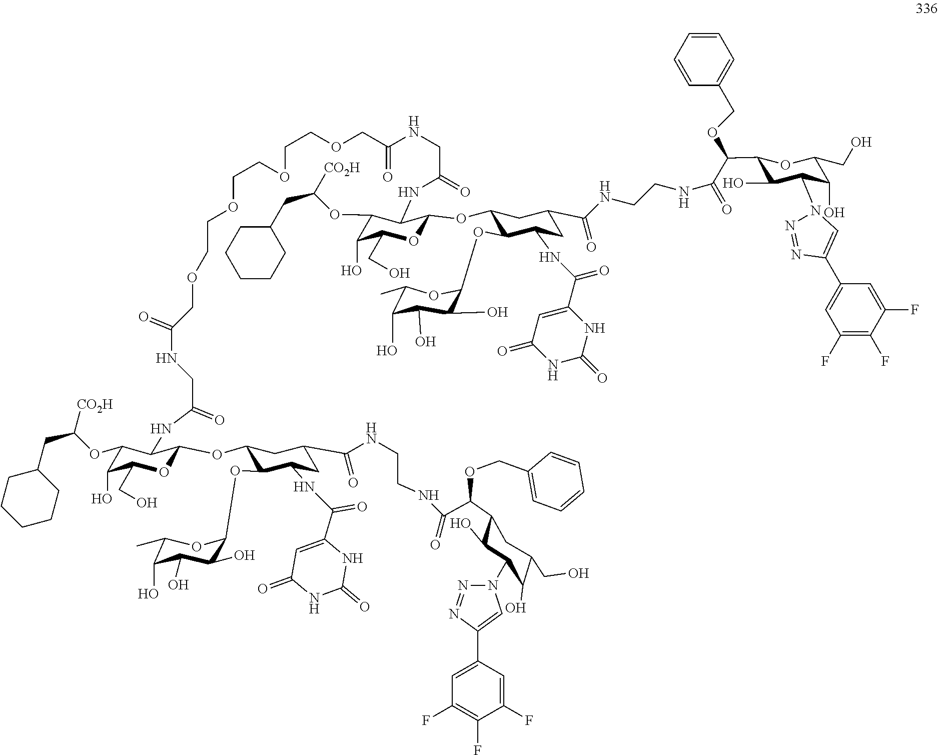

### Example 243

### Prophetic Synthesis of Multimeric Compound 337

Conpound 337: Compound 337 can be prepared in an analogous fashion to FIG. 14 by replacing compound 145 with compound 333.

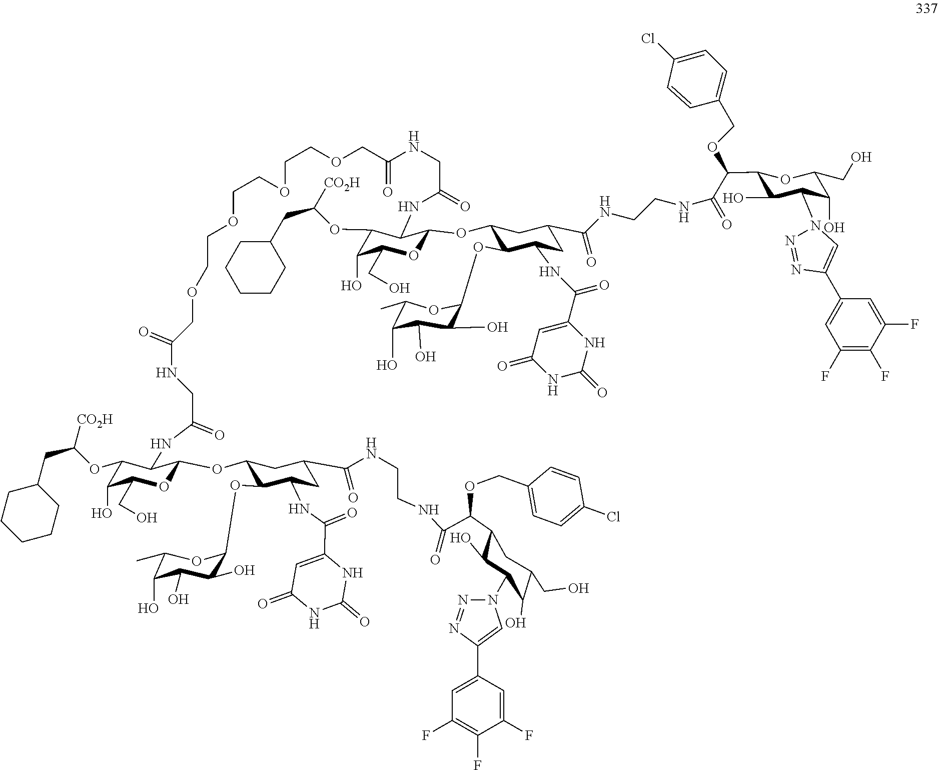

### Example 244

### Prophetic Synthesis of Multimeric Compound 338

Compound 338: Compound 338 can be prepared in an analogous fashion to FIG. 14 by replacing compound 145 with compound 334.

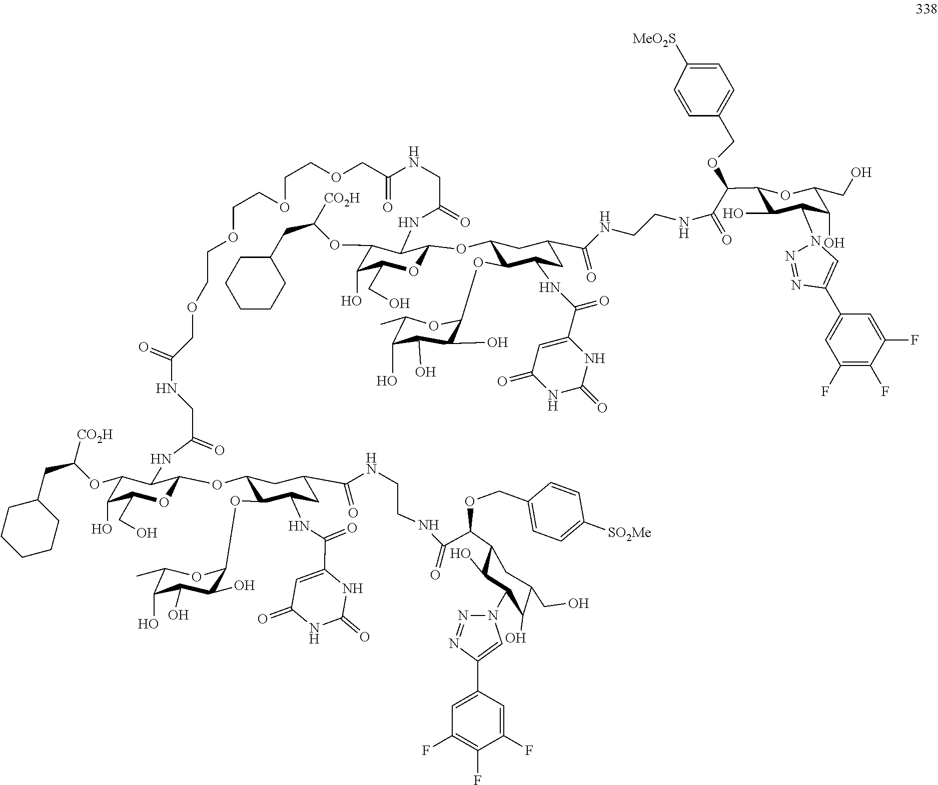

### Example 245

### Prophetic Synthesis of Multimeric Compound 339

Compound 339: Compound 339 can be prepared in an analogous fashion to FIG. 14 by replacing compound 145 with compound 335.

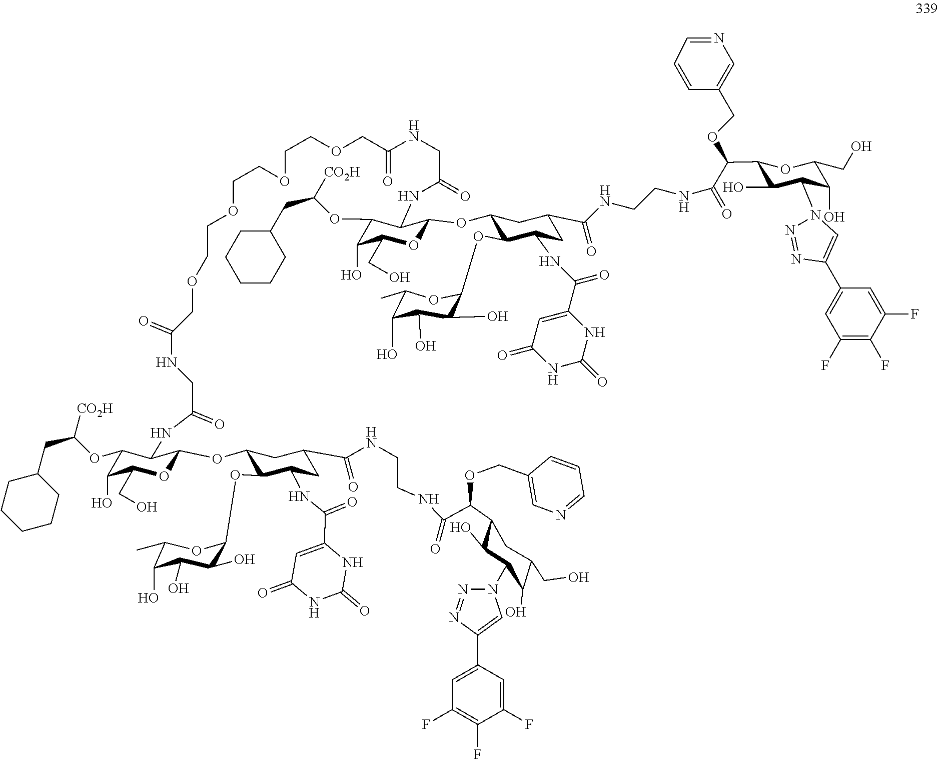

### Example 246

### Prophetic Synthesis of Multimeric Compound 340

Compound 340: Compound 340 can be prepared in an analogous fashion to FIG. 14 by replacing compound 22 with compound 40 and replacing compound 145 with compound 333.

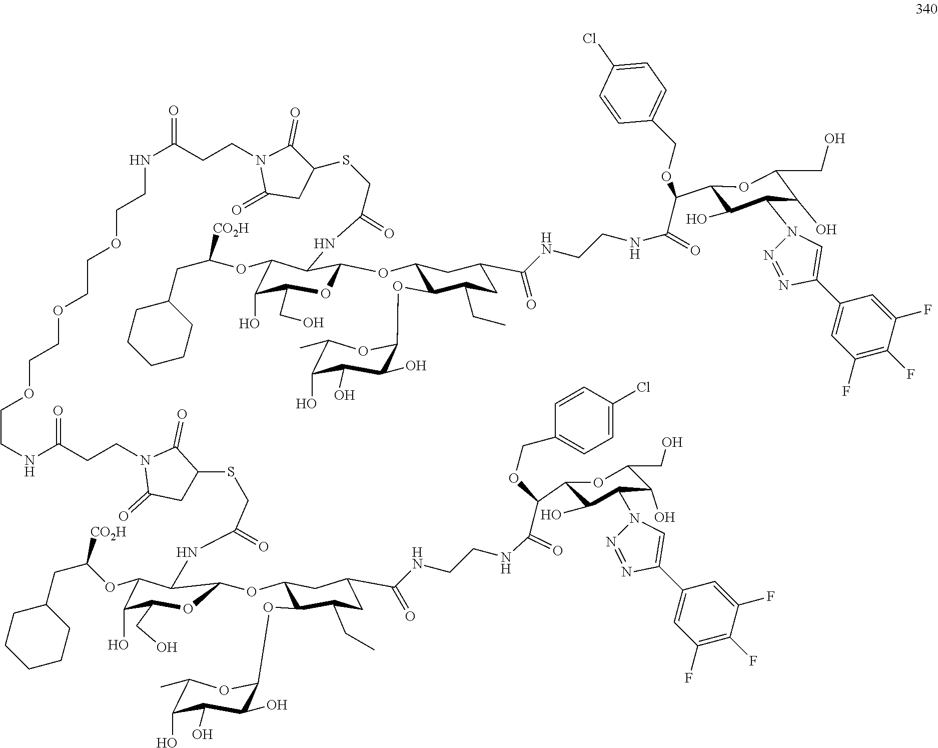

### Example 247

### Prophetic Synthesis of Multimeric Compound 341

Compound 341: Compound 341 can be prepared in an analogous fashion to FIG. 14 by replacing compound 22 with compound 78 and replacing compound 145 with compound 333.

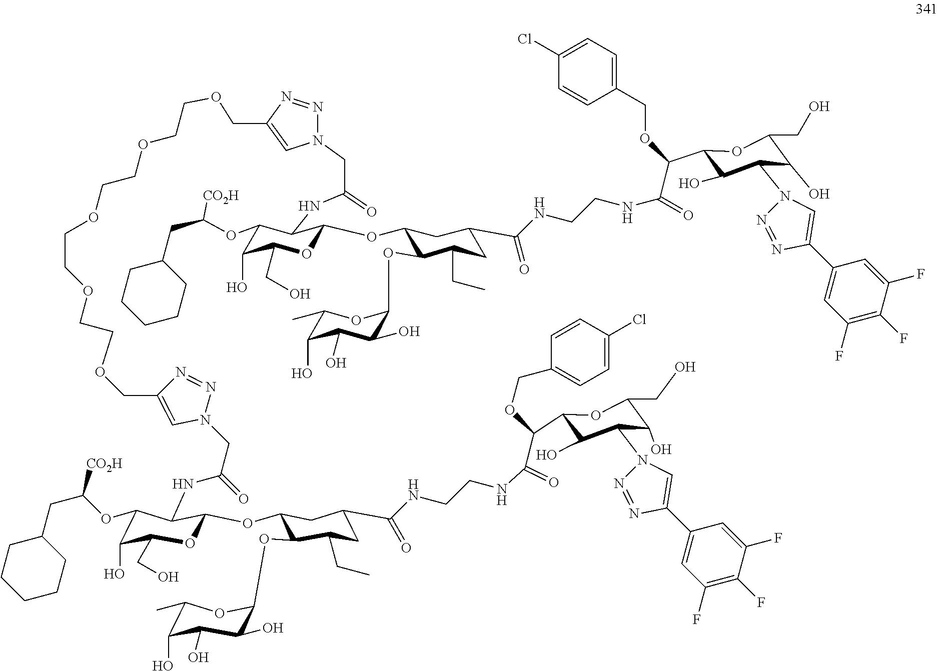

### Example 248

### Prophetic Synthesis of Multimeric Compound 342

Compound 342: Compound 342 can be prepared in an analogous fashion to FIG. 14 by replacing compound 22 with compound 87 and replacing compound 145 with compound 333.

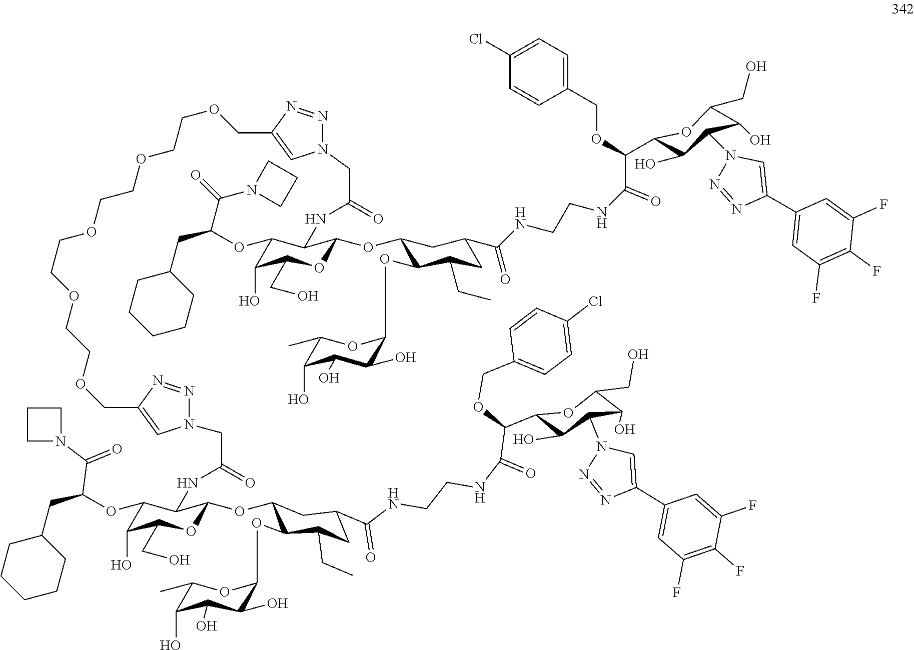

### Example 249

### Prophetic Synthesis of Multimeric Compound 343

Compound 343: Compound 342 can be prepared in an analogous fashion to FIG. 14 by replacing compound 22 with compound 88 and replacing compound 145 with compound 333.

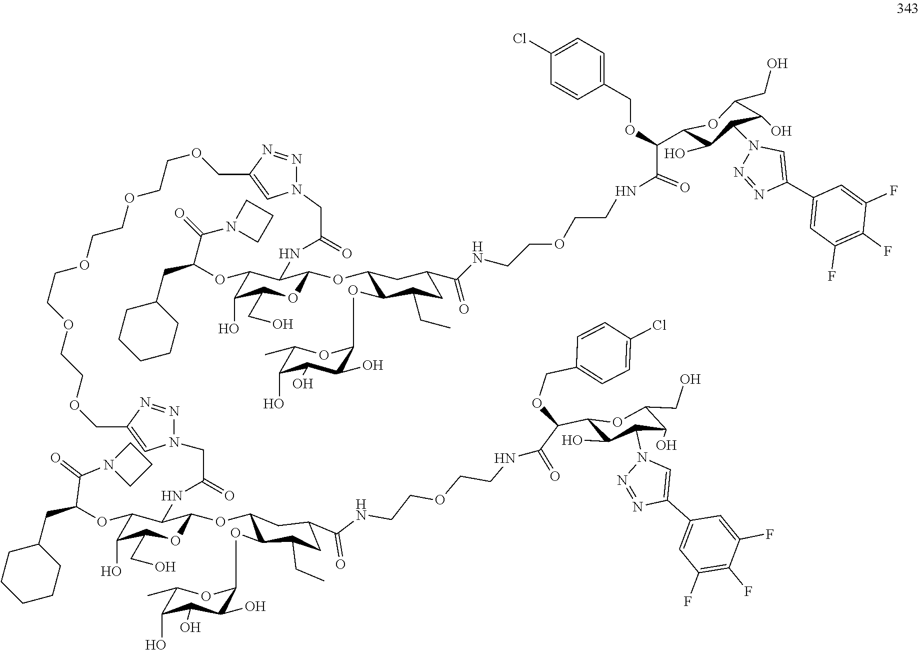

### Example 250

### E-Selectin Activity—Binding Assay

The inhibition assay to screen and characterize antagonists of E-selectin is a competitive binding assay, from which IC₅₀ values may be determined. E-selectin/Ig chimera are immobilized in 96 well microtiter plates by incubation at 37° C. for 2 hours. To reduce nonspecific binding, bovine serum albumin is added to each well and incubated at room temperature for 2 hours. The plate is washed and serial dilutions of the test compounds are added to the wells in the presence of conjugates of biotinylated, sLea polyacrylamide with streptavidin/horseradish peroxidase and incubated for 2 hours at room temperature.

To determine the amount of sLea bound to immobilized E-selectin after washing, the peroxidase substrate, 3,3′,5,5′ tetramethylbenzidine (TMB) is added. After 3 minutes, the enzyme reaction is stopped by the addition of H₃PO₄, and the absorbance of light at a wavelength of 450 nm is determined. The concentration of test compound required to inhibit binding by 50% is determined.

E-Selectin Antagonist Activity

| Compound | IC50 (nM) |
|---|---|
| Compound 206 | 1.6 |

### Example 251

### Galectin-3 Activity—ELISA Assay

Galectin-3 antagonists can be evaluated for their ability to inhibit binding of galectin-3 to a Galβ1-3GlcNAc carbohydrate structure. The detailed protocol is as follows. A 1 ug/mL suspension of a Galβ1-3GlcNAcβ1-3Galβ1-4GlcNAcβ-PAA-biotin polymer (Glycotech, catalog number 01-096) is prepared. A 100 uL aliquot of the polymer is added to the wells of a 96-well streptavidin-coated plate (R&D Systems, catalog number CP004). A 100 uL aliquot of 1× Tris Buffered Saline (TBS, Sigma, catalog number T5912-10X) is added to control wells. The polymer is allowed to bind to the streptavidin-coated wells for 1.5 hours at room temperature. The contents of the wells are discarded and 200 uL of 1× TBS containing 1% bovine serum albumin (BSA) is added to each well as a blocking reagent and the plate is kept at room temperature for 30 minutes. The wells are washed three times with 1× TBS containing 0.1% BSA. A serial dilution of test compounds is prepared in a separate V-bottom plate (Corning, catalog number 3897). A 75 uL aliquot of the highest concentration of the compound to be tested is added to the first well in a column of the V-bottom plate then 15 ul is serially transferred into 60 uL 1× TBS through the remaining wells in the column to generate a 1 to 5 serial dilution. A 60 uL aliquot of 2 ug/mL galectin-3 (IBL, catalog number IBATGP0414) is added to each well in the V-bottom plate. A 100 uL aliquot of the galectin-3/test compound mixture is transferred from the V-bottom plate into the assay plate containing the Galβ1-3GlcNAc polymer. Four sets of control wells in the assay plate are prepared in duplicate containing 1) both Galβ1-3GlcNAc polymer and galectin-3, 2) neither the polymer nor galectin-3, 3) galectin-3 only, no polymer, or 4) polymer only, no galectin-3. The plate is gently rocked for 1.5 hours at room temperature. The wells are washed four times with TBS/0.1% BSA. A 100 uL aliquot of anti-galectin-3 antibody conjugated to horse radish peroxidase (R&D Systems, from DGAL30 kit) is added to each well and the plate is kept at room temperature for 1 hour. The wells are washed four times with TBS/0.1% BSA. A 100 uL aliquot of TMB substrate solution is added to each well. The TMB substrate solution is prepared by making a 1:1 mixture of TMB Peroxidase Substrate (KPL, catalog number 5120-0048) and Peroxidase Substrate Solution B (KPL, catalog number 5120-0037). The plate is kept at room temperature for 10 to 20 minutes. The color development is stopped by adding 100 uL 10% phosphoric acid (RICCA Chemical Co., catalog number 5850-16). The absorbance at 450 nm (A₄₅₀) is measured using a FlexStation 3 plate reader (Molecular Devices). Plots of A₄₅₀ versus test compound concentration and IC₅₀ determinations are made using GraphPad Prism 6.

### Example 252

### CXCR4 Assay—Inhibition of Cyclic Amp

The CXCR4-cAMP assay measures the ability of a glycomimetic CXCR4 antagonist to inhibit the binding of CXCL12 (SDF-la) to CHO cells that have been genetically engineered to express CXCR4 on the cell surface. Assay kits may be purchased from DiscoveRx (95-0081E2CP2M; cAMP Hunter eXpress CXCR4 CHO-K1). The Gi-coupled receptor antagonist response protocol described in the kit instruction manual can be followed. GPCRs, such as CXCR4, are typically coupled to one of the 3 G-proteins: Gs, Gi or Gq. In the CHO cells supplied with the kit, CXCR4 is coupled to Gi. After activation of CXCR4 by ligand binding (CXCL12), Gi dissociates from the CXCR4 complex, becomes activated, and binds to adenylyl cyclase, thus inactivating it, resulting in decreased levels of intracellular cAMP Intracellular cAMP is usually low, so the decrease of the low level of cAMP by a Gi-coupled receptor will be hard to detect. Forskolin is added to the CHO cells to directly activate adenylyl cyclase (bypassing all GPCRs), thus raising the level of cAMP in the cell, so that a Gi response can be easily observed. CXCL12 interaction with CXCR4 decreases the intracellular level of cAMP and inhibition of CXCL12 interaction with CXCR4 by a CXCR4 antagonist increases the intracellular cAMP level, which is measured by luminescence.

What is claimed is:

1. At least one compound chosen from multimeric glycomimetic antagonists of Formula (I): 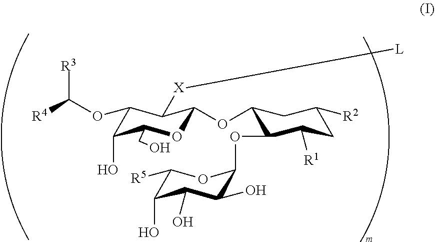
prodrugs of Formula (I), and pharmaceutically acceptable salts of any of the foregoing, wherein
each R¹, which may be identical or different, is independently chosen from H, C₁₋₁₂ alkyl, C₂₋₁₂ alkenyl, C₂₋₁₂ alkynyl, C₁₋₈ haloalkyl, C₂₋₈ haloalkenyl, C₂₋₈ haloalkynyl, 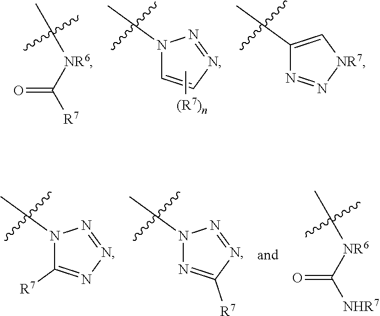
groups, wherein each n, which may be identical or different, is independently chosen from integers ranging from 0 to 2, each R⁶, which may be identical or different, is independently chosen from H, C₁₋₈ alkyl, C₂₋₈ alkenyl, C₂₋₈ alkynyl, C₄₋₁₆ cycloalkylalkyl, and —C(═O)R⁷ groups, and each R⁷, which may identical or different, is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
each R², which may be identical or different, is independently chosen from H, a non-glycomimetic moiety, and a linker-non-glycomimetic moiety, wherein each non-glycomimetic moiety, which may be identical or different, is independently chosen from galectin-3 inhibitors, CXCR4 chemokine receptor inhibitors, polyethylene glycol, thiazolyl, chromenyl, C₁₋₈ alkyl, R⁷, C₆₋₁₈ aryl-R⁸, C₁₋₁₂ heteroaryl-R⁸, 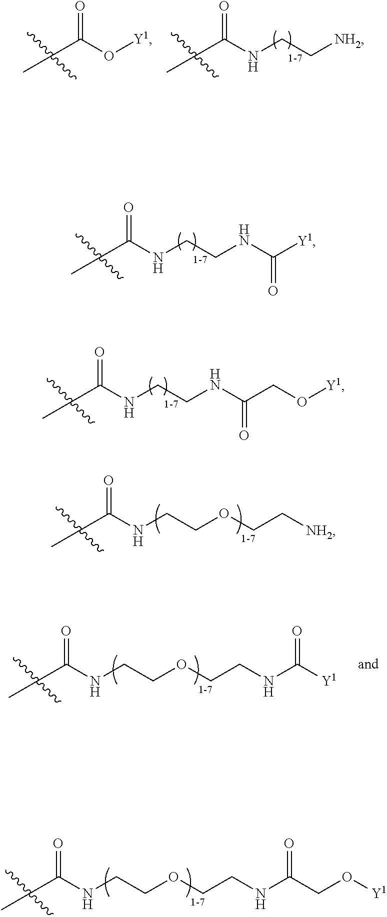
groups,
wherein each Y¹, which may be identical or different, is independently chosen from C₁₋₄ alkyl, C₂₋₄ alkenyl, and C₂₋₄ alkynyl groups and wherein each R⁸, which may be identical or different, is independently chosen from C₁₋₁₂ alkyl groups substituted with at least one substituent chosen from —OH, —OSO₃Q, —OPO₃Q₂, —CO₂Q, and —SO₃Q groups and C₂₋₁₂ alkenyl groups substituted with at least one substituent chosen from —OH, —OSO₃Q, —OPO₃Q₂, —CO₂Q, and —SO₃Q groups, wherein each Q, which may be identical or different, is independently chosen from H and pharmaceutically acceptable cations;
each R³, which may be identical or different, is independently chosen from —CN, —CH₂CN, and —C(═O)Y² groups, wherein each Y², which may be identical or different, is independently chosen from C₁₋₈ alkyl, C₂₋₈ alkenyl, C₂₋₈ alkynyl, —OZ¹, —NHOH, —NHOCH₃, —NHCN, and —NZ¹Z² groups, wherein each Z¹ and Z², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, and C₇₋₁₂ arylalkyl groups, wherein Z¹ and Z² may join together along with the nitrogen atom to which they are attached to form a ring;
each R⁴, which may be identical or different, is independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₄₋₁₆ cycloalkylalkyl, and C₆₋₁₈ aryl groups;
each R⁵, which may be identical or different, is independently chosen from —CN, C₁₋₁₂ alkyl, and C₁₋₁₂ haloalkyl groups;
each X, which may be identical or different, is independently chosen from —O— and —N(R⁹)—, wherein each R⁹, which may be identical or different, is independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
m is chosen from integers ranging from 2 to 256; and
L is independently chosen from linker groups.

2. The at least one compound according to claim 1, wherein at least one R² is H.

3. The at least one compound according to claim 1, wherein at least one R² is chosen from 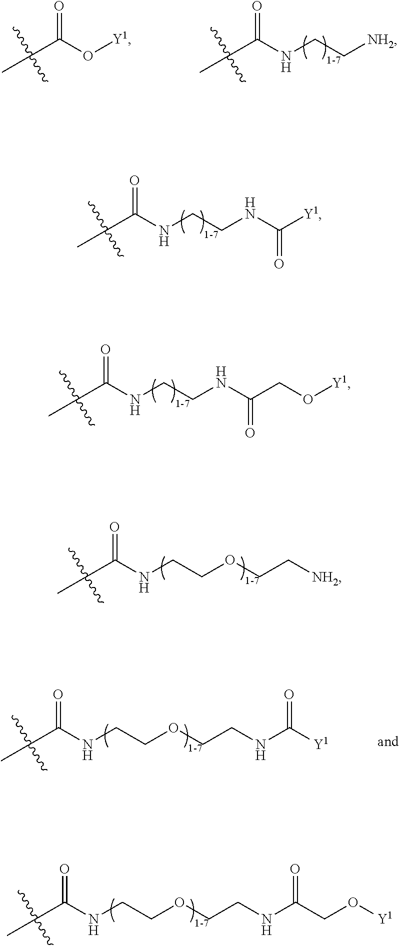
groups.

4. The at least one compound according to claim 3, wherein each Y¹ is methyl.

5. The at least one compound according to claim 1, wherein at least one R² is

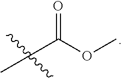 [SMILES: *C(=O)OC]

6. The at least one compound according to claim 1, wherein at least one R² is chosen from

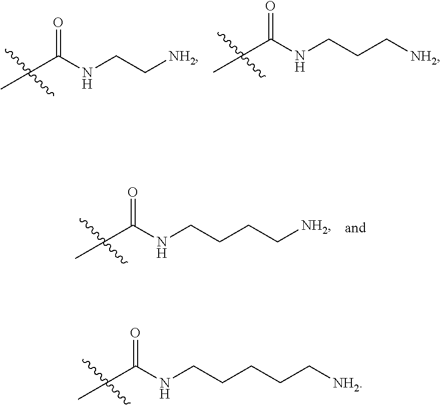 [SMILES: *C(=O)NCCN ; *C(=O)NCCCN ; *C(=O)NCCCCN ; *C(=O)NCCCCCN]

7. The at least one compound according to claim 1, wherein at least one R² is

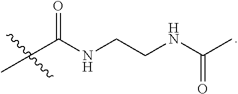 [SMILES: *C(=O)NCCNC(C)=O]

8. The at least one compound according to claim 1, wherein at least one R² is

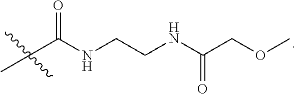 [SMILES: *C(=O)NCCNC(=O)COC]

9. The at least one compound according to claim 1, wherein at least one R² is chosen from a linker-non-glycomimetic moiety, wherein the non-glycomimetic moiety is chosen from galectin-3 inhibitors.

10. The at least one compound according to claim 9, wherein at least one galectin-3 inhibitor is chosen from 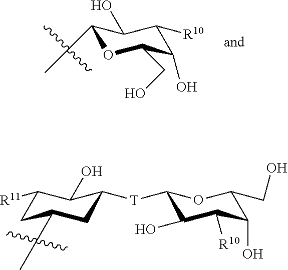
groups, wherein each T, which may be identical or different, is independently chosen from —O— and —S—, and each R¹⁰ and each R¹¹, which may be identical or different, are independently chosen from C₆₋₁₈ aryl, C₁₋₁₃ heteroaryl, C₇₋₁₉ arylalkyl, C₇₋₁₉ arylalkoxy, C₂₋₁₄ heteroarylalkyl, C₂₋₁₄ heteroarylalkoxy, and —NHC(═O)Y³ groups, wherein each Y³, which may be identical or different, is independently chosen from C₁₋₈ alkyl, C₂₋₁₂ heterocyclyl, C₆₋₁₈ aryl, and C₁₋₁₃ heteroaryl groups.

11. The at least one compound according to claim 10, wherein the at least one galectin-3 inhibitor is chosen from 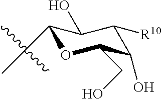
groups.

12. The at least one compound according to claim 10, wherein the at least one galectin-3 inhibitor is chosen from 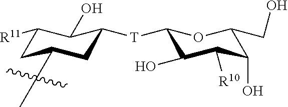
groups.

13. The at least one compound according to any one of claims 10 or 12, wherein at least one T is —O—.

14. The at least one compound according to any one of claims 10 or 12, wherein at least one T is —S—.

15. The at least one compound according to any one of claims 10-14, wherein at least one R¹⁰ or at least one R¹¹ is chosen from 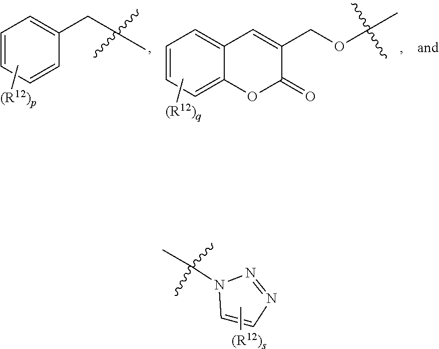
groups,
wherein each p, which may be identical or different, is independently chosen from integers ranging from 0 to 5, each q, which may be identical or different, is independently chosen from integers ranging from 0 to 4, each s, which may be identical or different, is independently chosen from integers ranging from 0 to 2, and wherein each R², which may be identical or different, is independently chosen from H, halo, —OH, —OY⁴, —OC(═O)Y⁴, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups, wherein each Y⁴, which may be identical or different, is independently chosen from C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₆₋₁₈ aryl, and C₁₋₁₃ heteroaryl groups.

16. The at least one compound according to claim 15, wherein at least one R¹⁰ or at least one R¹¹ is

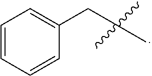 [SMILES: *Cc1ccccc1]

17. The at least one compound according to claim 15, wherein at least one R¹⁰ or at least one R¹¹ is

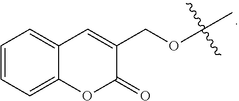 [SMILES: *OCc1cc2ccccc2oc1=O]

18. The at least one compound according to claim 15, wherein at least one R¹⁰ or at least one R¹¹ is

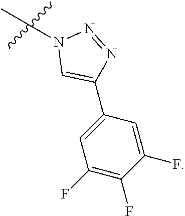 [SMILES: *n1cc(-c2cc(F)c(F)c(F)c2)nn1]

19. The at least one compound according to claim 9, wherein at least one galectin-3 inhibitor is

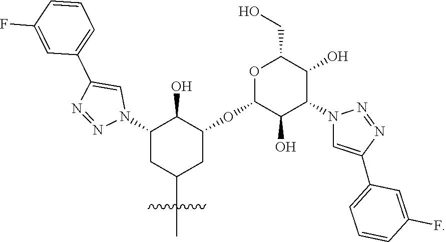 [SMILES: *C1C[C@@H](O[C@@H]2O[C@H](CO)[C@H](O)[C@H](n3cc(-c4cccc(F)c4)nn3)[C@H]2O)[C@H](O)[C@@H](n2cc(-c3cccc(F)c3)nn2)C1]

20. The at least one compound according to claim 9, wherein at least one galectin-3 inhibitor is chosen from 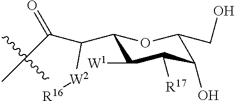
groups,
wherein
each W¹, which may be identical or different, is independently chosen from —O—, —S—, —C—, and —N(R¹⁵)—, wherein each R¹⁵, which may be identical or different, is independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups;
each W², which may be identical or different, is independently chosen from H, halo, and —OZ³ groups, wherein each Z³, which may be identical or different, is independently chosen from H and C₁₋₈ alkyl groups;
each R¹⁶, which may be identical or different, is independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, C₆₋₁₈ aryl, C₁₋₁₃ heteroaryl, C₁₋₁₈ arylalkyl, and C₂₋₁₄ heteroarylalkyl groups, wherein the C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, C₆₋₁₈ aryl, C₁₋₈ heteroaryl, C₇₋₁₉ arylalkyl, and C₂₋₁₄ heteroarylalkyl groups are optionally substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, —C(═O)NZ⁴Z⁵, and —SO₂Z⁴ groups, wherein each of Z⁴ and Z⁵, which may be identical or different, are independently chosen from H, C₁₋₈ alkyl, and C₁₋₈ haloalkyl groups, or Z¹ and Z⁵ join together along with the nitrogen atom to which they are attached to form a ring;
each R¹⁷, which may be identical or different, is independently chosen from C₆₋₁₈ aryl and C₁₋₁₃ heteroaryl groups, wherein the C₆₋₁₈ aryl and C₁₋₁₃ heteroaryl groups are optionally substituted with one or more groups independently chosen from R¹⁸, C₁₋₈ alkyl, C₁₋₈ haloalkyl, —C(═O)OZ⁶, and —C(═O)NZ⁶Z¹ groups, wherein each R¹⁸, which may be identical or different, is independently chosen from C₆₋₁₈ aryl groups optionally substituted with one or more groups independently chosen from halo, C₁₋₈ alkyl, —OZ⁸, —C(═O)OZ⁸, and —C(═O)NZ⁸Z⁹ groups, wherein each Z⁶, each Z⁷, each Z⁸ and each Z⁹, which may be identical or different, are independently chosen from H and C₁₋₈ alkyl groups, or Z⁶ and Z⁷ join together along with the nitrogen atom to which they are attached to form a ring and/or Z⁸ and Z⁹ join together along with the nitrogen atom to which they are attached to form a ring; and
wherein each of Z³, Z⁴, Z⁵, Z⁶, Z⁷, Z⁸, and Z⁹ is optionally substituted with one or more groups independently chosen from halo and —OR¹⁹ groups, wherein R¹⁹ is independently chosen from H and C₁₋₈ alkyl groups.

21. The at least one compound according to claim 20, wherein at least one R¹⁶ is chosen from

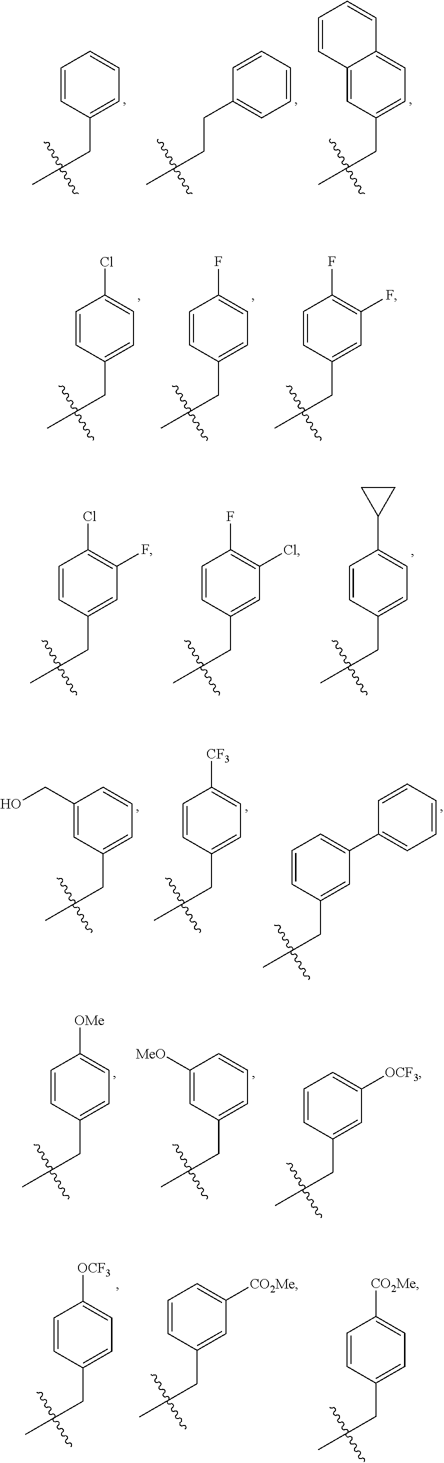

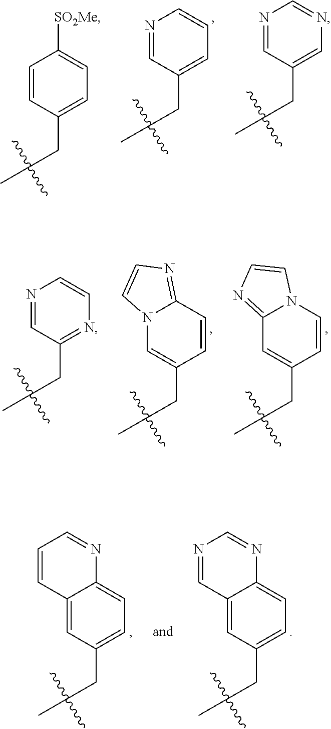

22. The at least one compound according to claims 20 or 21, wherein at least one R¹⁷ is chosen from

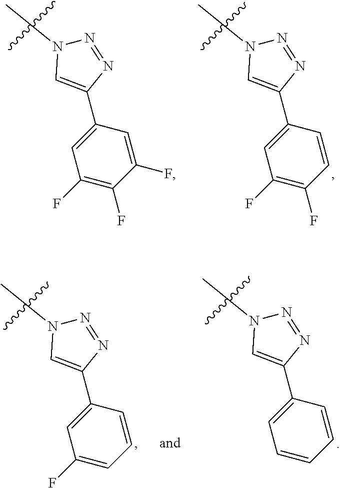 [SMILES: *n1cc(-c2cc(F)c(F)c(F)c2)nn1 ; *n1cc(-c2ccc(F)c(F)c2)nn1 ; *n1cc(-c2cccc(F)c2)nn1 ; *n1cc(-c2ccccc2)nn1]

23. The at least one compound according to any one of claims 20-22, wherein the at least one galectin-3 inhibitor is chosen from 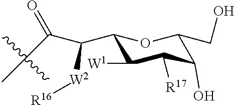
groups.

24. The at least one compound according to any one of claims 20-22, wherein the at least one galectin-3 inhibitor is chosen from 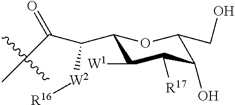
groups.

25. The at least one compound according to any one of claims 20-24, wherein W¹ is —OH.

26. The at least one compound according to any one of claims 20-25, wherein W² is —C—.

27. The at least one compound according to any one of claims 20-25, wherein W² is —O—.

28. The at least one compound according to any one of claims 20-25, wherein W² is —S—.

29. The at least one compound according to claim 1, wherein at least one R² is chosen from a linker-non-glycomimetic moiety, wherein the non-glycomimetic moiety is chosen from CXCR4 chemokine receptor inhibitors.

30. The at least one compound according to claim 29, wherein the CXCR4 chemokine receptor inhibitor is chosen from 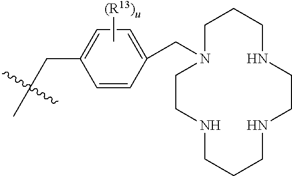
groups, wherein each R¹³, which may be identical or different, is independently chosen from H, halo, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups and wherein u is chosen from integers ranging from 1 to 4.

31. The at least one compound according to claim 29, wherein the CXCR4 chemokine receptor inhibitor is chosen from 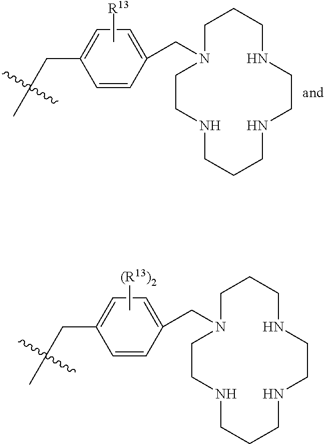
groups,
wherein each R¹³, which may be identical or different, is independently chosen from H and halo groups.

32. The at least one compound according to claims 30 or 31, wherein at least one R³ is H.

33. The at least one compound according to claims 30 or 31, wherein at least one R³ is chosen from halo groups.

34. The at least one compound according to claim 33, wherein at least one R³ is bromo.

35. The at least one compound according to claim 29, wherein the CXCR4 chemokine receptor inhibitor is

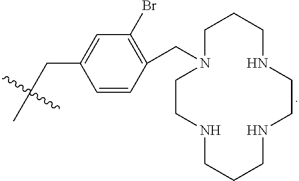 [SMILES: *Cc1ccc(CN2CCCNCCNCCCNCC2)c(Br)c1]

36. The at least one compound according to claim 1, wherein at least one R² is chosen from a linker-non-glycomimetic moiety, wherein the non-glycomimetic moiety is chosen from R⁸, C₆₋₁₈ aryl-R⁸, and C₁₋₁₂ heteroaryl-R⁸ groups.

37. The at least one compound according to claim 36, wherein the non-glycomimetic moiety is chosen from R⁸ groups.

38. The at least one compound according to claim 36, wherein the non-glycomimetic moiety is chosen from C₆₋₁₈ aryl-R⁸ groups.

39. The at least one compound according to claim 36, wherein the non-glycomimetic moiety is chosen from C₁₋₁₂ heteroaryl-R⁸ groups.

40. The at least one compound according to claim 36, wherein the non-glycomimetic moiety is chosen from 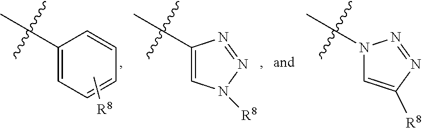
groups.

41. The at least one compound according to any one of claims 36-40, wherein R⁸ is chosen from C₁₋₅ alkyl groups substituted with at least one substituent chosen from —OH, —OSO₃Q, —OPO₃Q₂, —CO₂Q, and —SO₃Q groups.

42. The at least one compound according to any one of claims 36-40, wherein R⁸ is chosen from C₁₋₅ alkyl groups substituted with one, two, or three —OH groups.

43. The at least one compound according to any one of claims 36-40, wherein R⁸ is chosen from C₁₋₅ alkyl groups substituted with one, two, or three substituents independently chosen from —OSO₃Q groups.

44. The at least one compound according to any one of claims 36-40, wherein R⁸ is chosen from C₁₋₅ alkyl groups substituted with one, two, or three substituents independently chosen from —SO₃Q groups.

45. The at least one compound according to any one of claims 36-40, wherein R⁸ is chosen from C₁₋₈ alkyl groups substituted with one, two, or three substituents independently chosen from —CO₂Q groups.

46. The at least one compound according to any one of claims 36-40, wherein R⁸ is chosen from C₂₋₅ alkenyl groups substituted with at least one substituent chosen from —OH, —OSO₃Q, —OPO₃Q₂, —CO₂Q, and —SO₃Q groups.

47. The at least one compound according to any one of claims 36-40, wherein R⁸ is chosen from C₂₋₅ alkenyl groups substituted with one substituent chosen from —OH, —OSO₃Q, —OPO₃Q₂, —CO₂Q, and —SO₃Q groups.

48. The at least one compound according to any one of claims 36-40, wherein R⁸ is chosen from C₂₋₅ alkenyl groups substituted with one, two, or three substituents independently chosen from —SO₃Q groups.

49. The at least one compound according to any one of claims 36-40, wherein R⁸ is chosen from 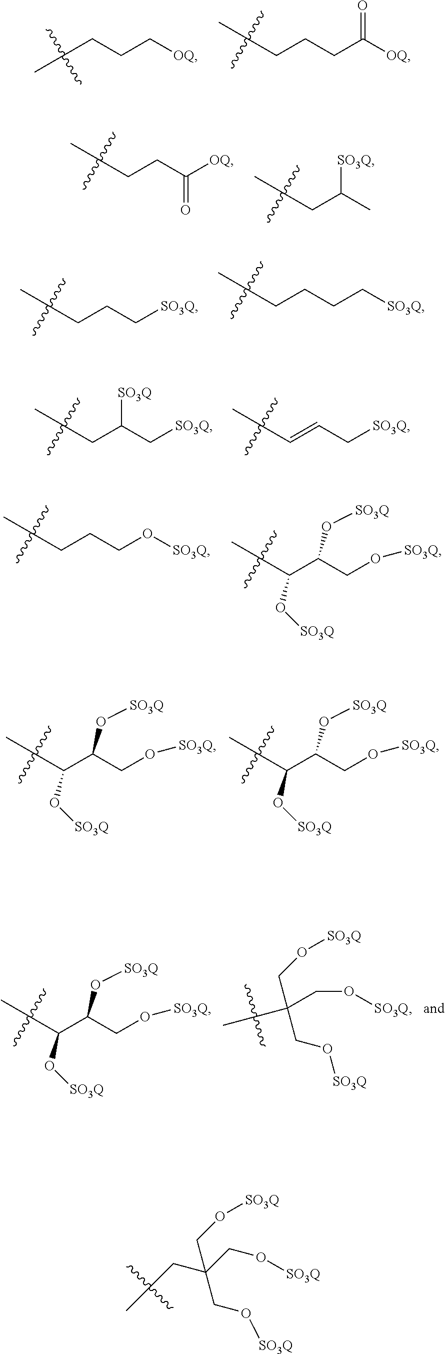
groups.

50. The at least one compound according to claim 36, wherein at least one R² is chosen from a linker-non-glycomimetic moiety, wherein the non-glycomimetic moiety is chosen from 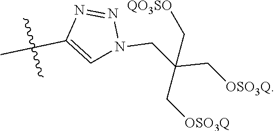
wherein each Q, which may be identical or different, is independently chosen from H and pharmaceutically acceptable cations.

51. The at least one compound according to any one of claims 41-50, wherein each Q is H.

52. The at least one compound according to any one of claims 41-50, wherein each Q is independently chosen from pharmaceutically acceptable cations.

53. The at least one compound according to claim 52, wherein each Q is a sodium cation.

54. The at least one compound according to claim 1, wherein at least one R² is chosen from a linker-non-glycomimetic moiety, wherein the non-glycomimetic moiety is chosen from polyethylene glycol, thiazolyl, and chromenyl groups.

55. The at least one compound according to claim 54, wherein the non-glycomimetic moiety is chosen from polyethylene glycol groups.

56. The at least one compound according to claim 55, wherein the polyethylene glycol is chosen from 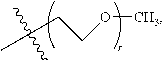
wherein r is chosen from integers ranging from 1 to 100.

57. The at least one compound according to claim 56, wherein r is chosen from integers ranging from 1 to 25.

58. The at least one compound according to claim 56, wherein r is chosen from integers ranging from 2 to 20.

59. The at least one compound according to claim 56, wherein r is 16.

60. The at least one compound according to claim 56, wherein r is 12.

61. The at least one compound according to claim 56, wherein r is 8.

62. The at least one compound according to claim 56, wherein r is 4.

63. The at least one compound according to claim 56, wherein the non-glycomimetic moiety is chosen from thiazolyl groups.

64. The at least one compound according to claim 53, wherein the non-glycomimetic moiety is

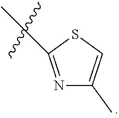 [SMILES: *c1nc(C)cs1]

65. The at least one compound according to claim 54, wherein the non-glycomimetic moiety is chosen from chromenyl groups.

66. The at least one compound according to claim 55, wherein the non-glycomimetic moiety is

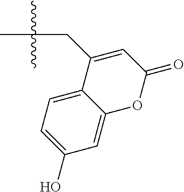 [SMILES: *Cc1cc(=O)oc2cc(O)ccc12]

67. The at least one compound according to any preceding claim, wherein at least one R¹ is chosen from H, methyl, and ethyl.

68. The at least one compound according to claim 67, wherein at least one R¹ is methyl.

69. The at least one compound according to claim 67, wherein at least one R¹ is ethyl.

70. The at least one compound according to any one of claims 1-66, wherein at least one R¹ is chosen from 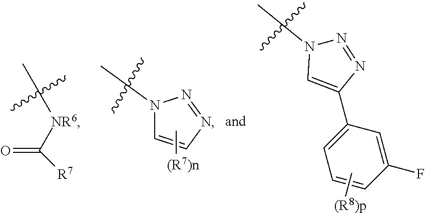
groups.

71. The at least one compound according to any one of claims 1-66, wherein at least one R¹ is chosen from

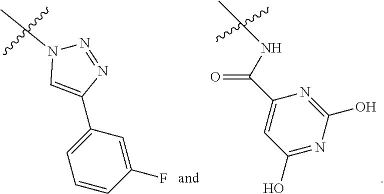 [SMILES: *n1cc(-c2cccc(F)c2)nn1 ; *NC(=O)c1cc(O)nc(O)n1]

72. The at least one compound according to claim 71, wherein at least one R¹ is

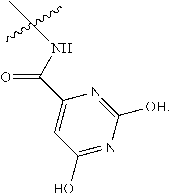 [SMILES: *NC(=O)c1cc(O)nc(O)n1]

73. The at least one compound according to any preceding claim, wherein at least one R³ is chosen from —C(═O)Y¹ groups.

74. The at least one compound according to claim 73, wherein at least one R³ is chosen from

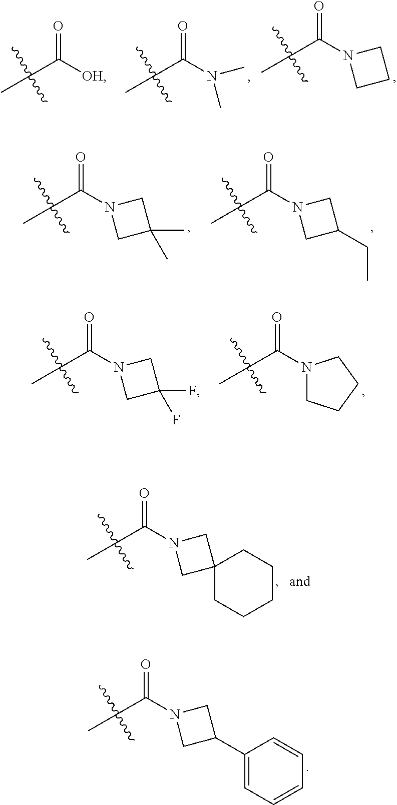

75. The at least one compound according to claim 74, wherein at least one R³ is chosen from

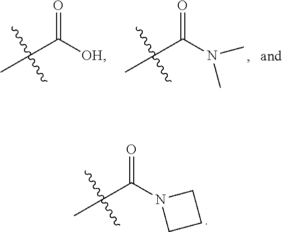

76. The at least one compound according to claim 75, wherein at least one R³ is

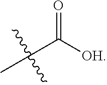 [SMILES: *C(=O)O]

77. The at least one compound according to any preceding claim, wherein at least one R⁴ is chosen from C₁₋₈ alkyl and C₄₋₁₆ cycloalkylalkyl groups.

78. The at least one compound according to claim 77, wherein at least one R³⁷ is chosen from propyl and cyclohexylmethyl.

79. The at least one compound according to claim 78, wherein at least one R⁴ is cyclohexylmethyl.

80. The at least one compound according to any preceding claim, wherein at least one R⁵ is chosen from —CN, CF₃, and methyl.

81. The at least one compound according to claim 80, wherein at least one R⁵ is methyl.

82. The at least one compound according to any preceding claim, wherein at least one X is chosen from —O— and —NH—.

83. The at least one compound according to claim 82, wherein at least one X is —O—.

84. The at least one compound according to claim 82, wherein at least one X is —NH—.

85. The at least one compound according to any preceding claim, wherein at least one linker is chosen from 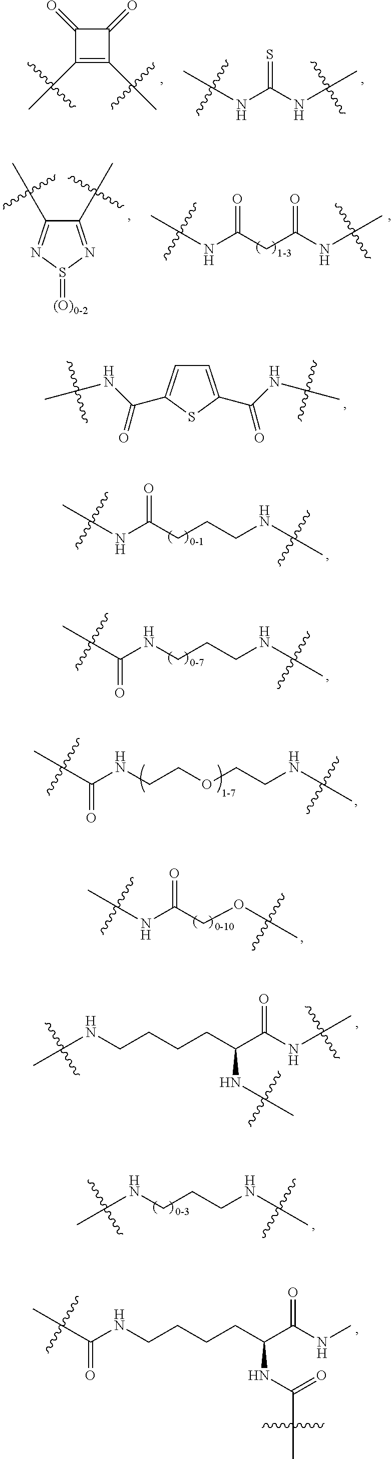 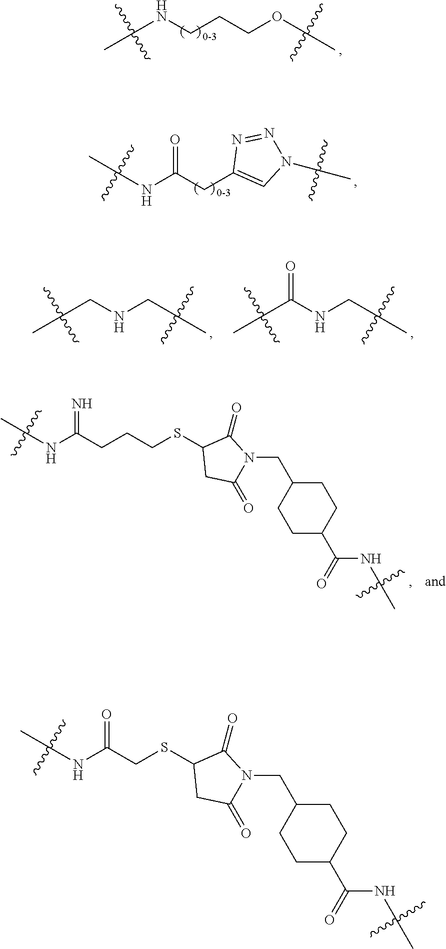
groups.

86. The at least one compound according to any one of claims 1-84, wherein at least one linker is chosen from

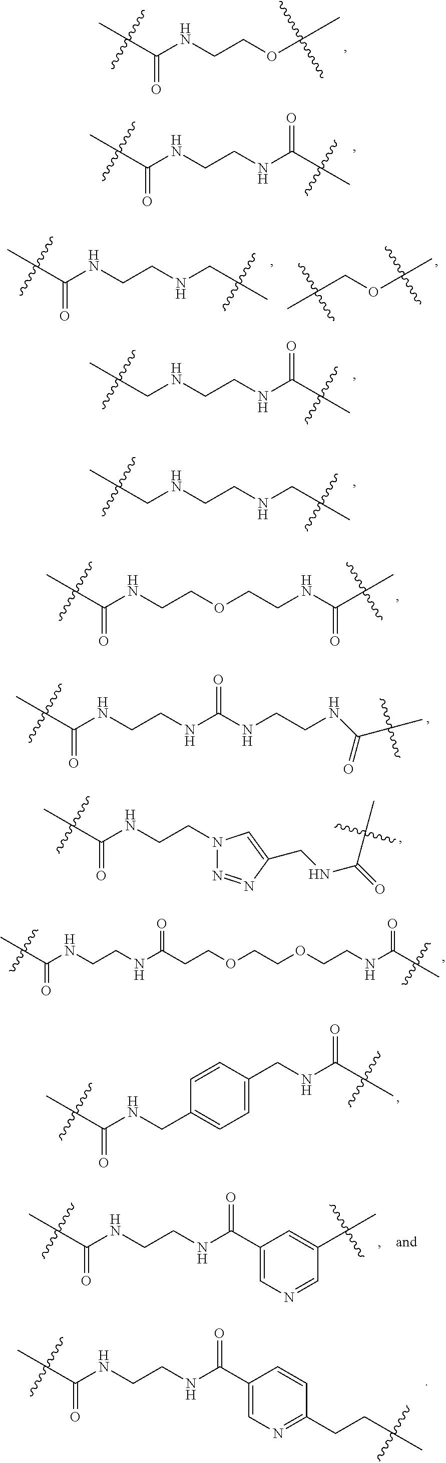

87. The at least one compound according to any one of claims 1-84, wherein at least one linker is chosen from

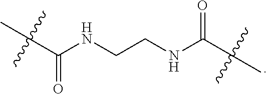 [SMILES: *C(=O)NCCNC(*)=O]

88. The at least one compound according to any one of claims 1-84, wherein at least one linker is chosen from

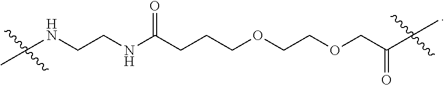 [SMILES: *NCCNC(=O)CCCOCCOCC(*)=O]

89. The at least one compound according to any one of claims 1-84, wherein at least one linker is chosen from

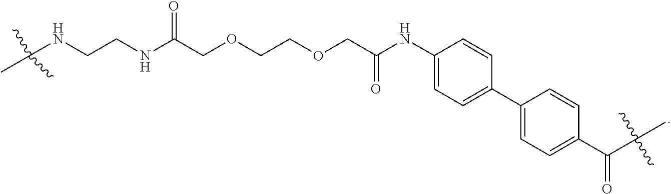 [SMILES: *NCCNC(=O)COCCOCC(=O)Nc1ccc(-c2ccc(C(*)=O)cc2)cc1]

90. The at least one compound according to any one of claims 1-84, wherein at least one linker is chosen from

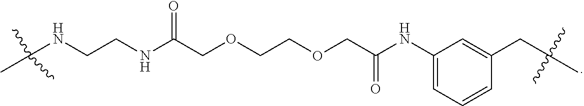 [SMILES: *Cc1cccc(NC(=O)COCCOCC(=O)NCCN*)c1]

91. The at least one compound according to any preceding claim, wherein m is chosen from integers ranging from 2 to 128.

92. The at least one compound according to any one of claims 1-90, wherein m is chosen from integers ranging from 2 to 32.

93. The at least one compound according to any one of claims 1-90, wherein m is chosen from integers ranging from 2 to 16.

94. The at least one compound according to any one of claims 1-90, wherein m is chosen from integers ranging from 2 to 8.

95. The at least one compound according to claim 94, wherein m is 6 and L is

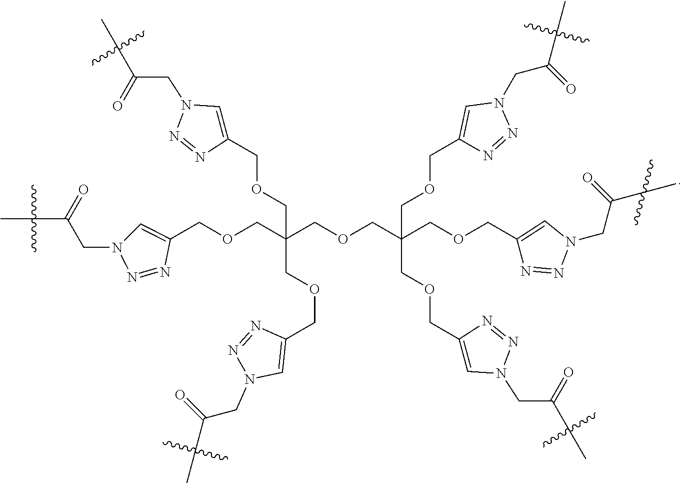 [SMILES: *C(=O)Cn1cc(COCC(COCc2cn(CC(*)=O)nn2)(COCc2cn(CC(*)=O)nn2)COCC(COCc2cn(CC(*)=O)nn2)(COCc2cn(CC(*)=O)nn2)COCc2cn(CC(*)=O)nn2)nn1]

96. The at least one compound according to ay one of claims 1-90, wherein m is chosen from integers ranging from 2 to 4.

97. The at least one compound according to any one of claims 1-90, wherein m is 4.

98. The at least one compound according to claim 97, wherein L is chosen from

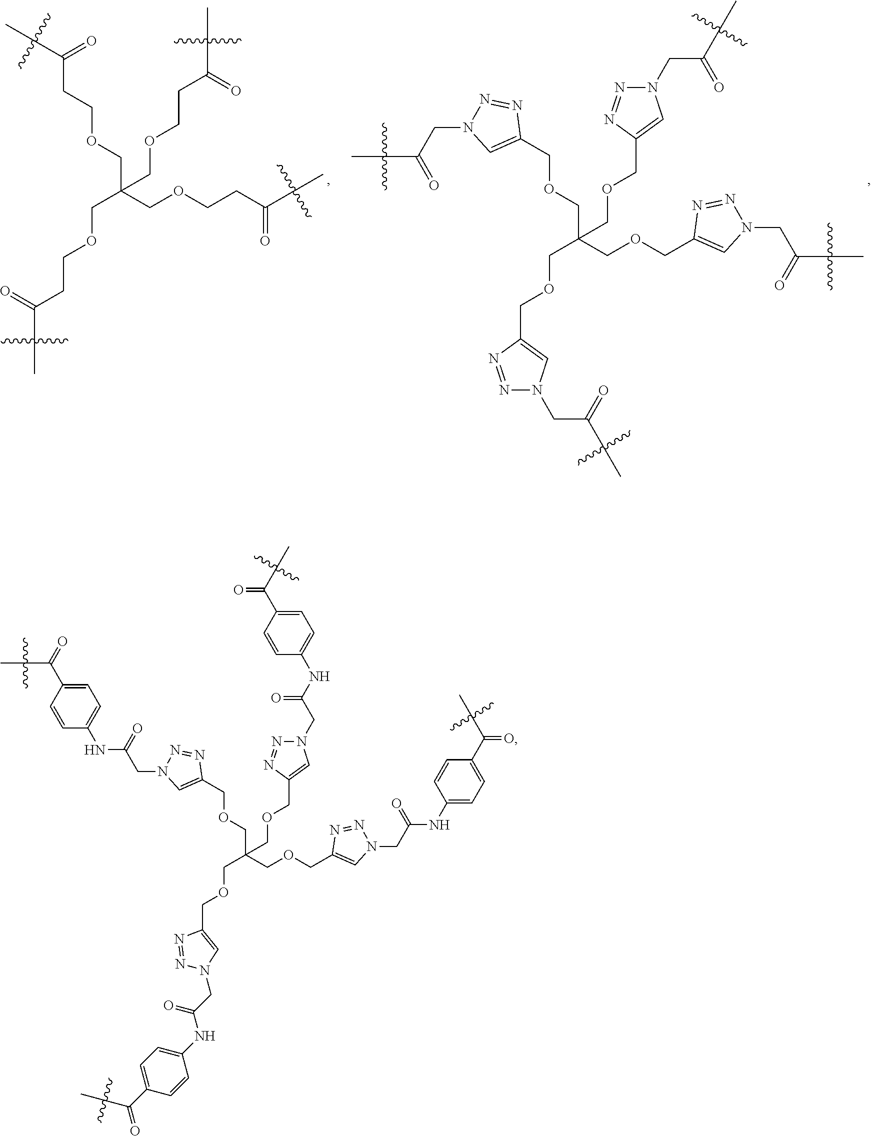 [SMILES: *C(=O)CCOCC(COCCC(*)=O)(COCCC(*)=O)COCCC(*)=O ; *C(=O)Cn1cc(COCC(COCc2cn(CC(*)=O)nn2)(COCc2cn(CC(*)=O)nn2)COCc2cn(CC(*)=O)nn2)nn1 ; *C(=O)c1ccc(NC(=O)Cn2cc(COCC(COCc3cn(CC(=O)Nc4ccc(C(*)=O)cc4)nn3)(COCc3cn(CC(=O)Nc4ccc(C(*)=O)cc4)nn3)COCc3cn(CC(=O)Nc4ccc(C(*)=O)cc4)nn3)nn2)cc1]

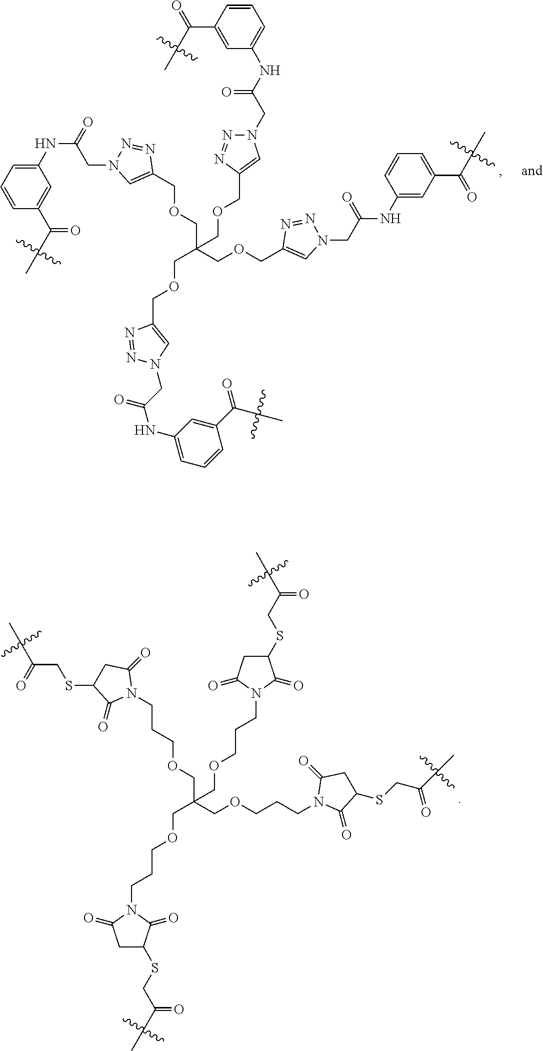 [SMILES: *C(=O)c1cccc(NC(=O)Cn2cc(COCC(COCc3cn(CC(=O)Nc4cccc(C(*)=O)c4)nn3)(COCc3cn(CC(=O)Nc4cccc(C(*)=O)c4)nn3)COCc3cn(CC(=O)Nc4cccc(C(*)=O)c4)nn3)nn2)c1 ; *C(=O)CSC1CC(=O)N(CCCOCC(COCCCN2C(=O)CC(SCC(*)=O)C2=O)(COCCCN2C(=O)CC(SCC(*)=O)C2=O)COCCCN2C(=O)CC(SCC(*)=O)C2=O)C1=O]

99. The at least one compound according to claim 97, wherein L is chosen from 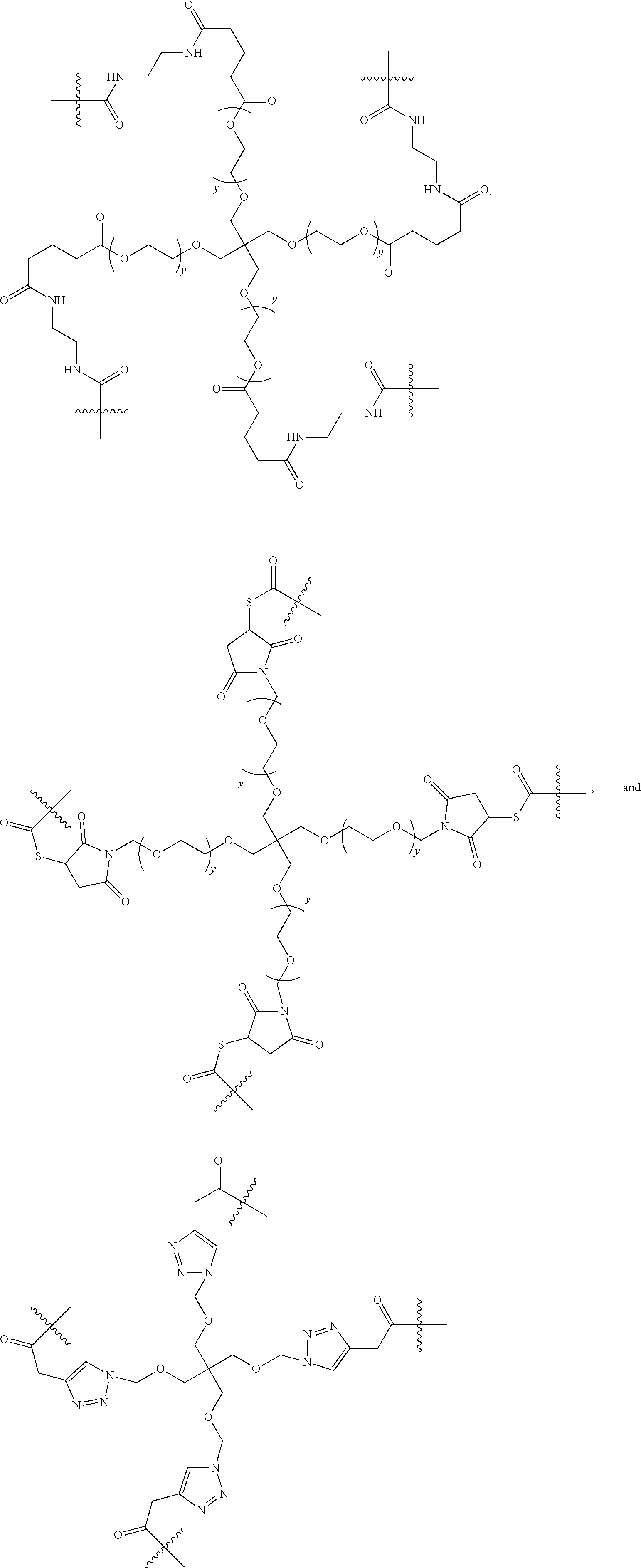
groups
wherein each y, which may be identical or different, is independently chosen from integers ranging from 0 to 250.

100. The at least one compound according to any one of claims 1-90, wherein m is 3.

101. The at least one compound according to claim 100, wherein L is chosen from

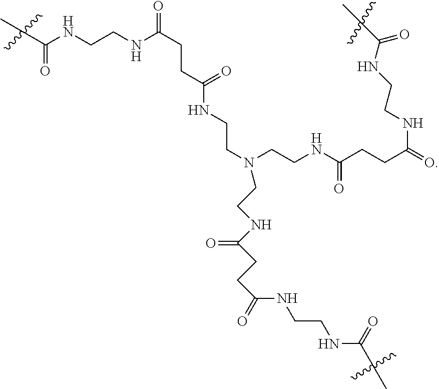 [SMILES: *C(=O)NCCNC(=O)CCC(=O)NCCN(CCNC(=O)CCC(=O)NCCNC(*)=O)CCNC(=O)CCC(=O)NCCNC(*)=O]

102. The at least one compound according to any one of claims 1-90, wherein m is 2.

103. The at least one compound according to claim 102, wherein L is chosen from 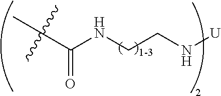
groups
wherein U is chosen from 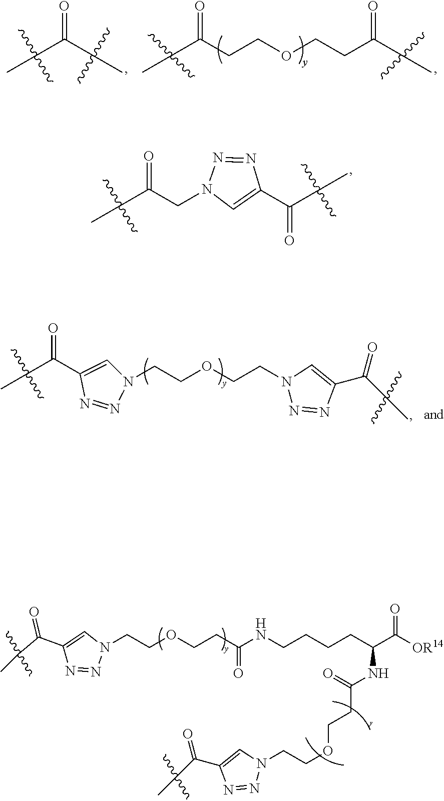
groups
wherein R¹⁴ is chosen from H, C₁₋₈ alkyl, C₆₋₁₈ aryl, C₇₋₁₉ arylalkyl, and C₁₋₁₃ heteroaryl groups and each y, which may be identical or different, is independently chosen from integers ranging from 0 to 250.

104. The at least one compound according to claim 103, wherein R¹⁴ is chosen from C₁₋₈ alkyl groups.

105. The at least one compound according to any one of claims 1-102, wherein L is chosen from 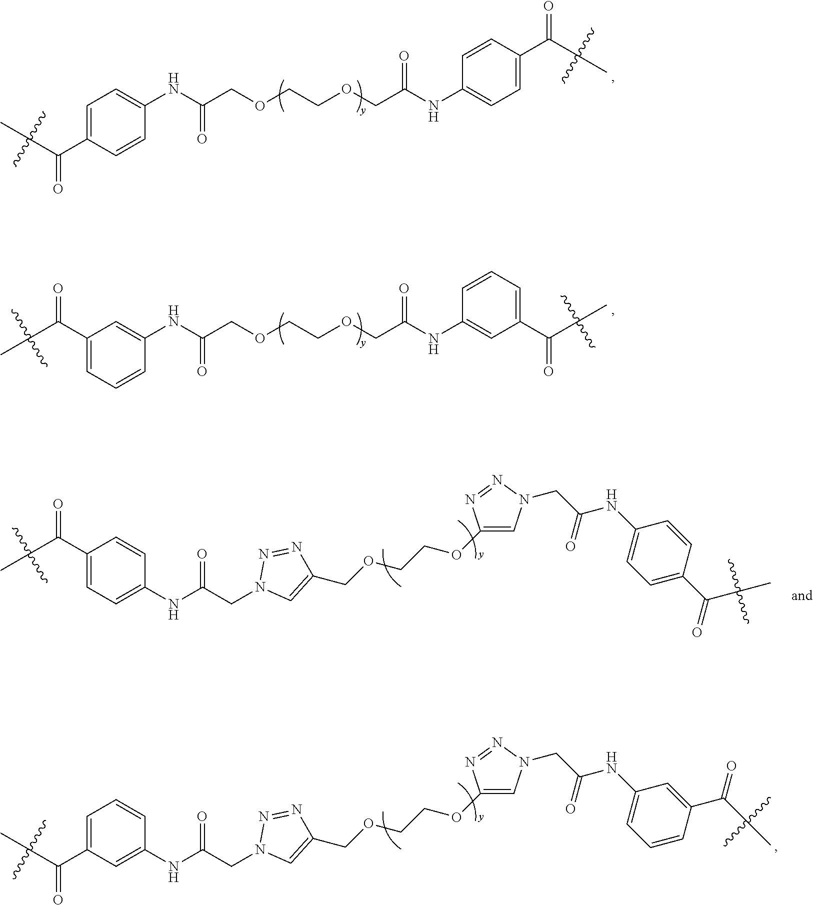
wherein y is chosen from integers ranging from 0 to 250.

106. The at least one compound according to any one of claims 1-102, wherein L is chosen from 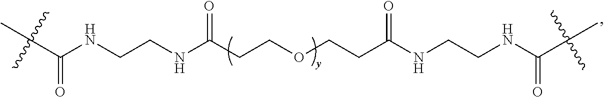
wherein y is chosen from integers ranging from 0 to 250.

107. The at least one compound according to any one of claims 1-102, wherein L is chosen from

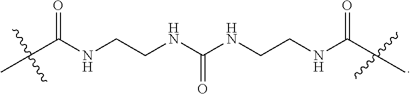 [SMILES: *C(=O)NCCNC(=O)NCCNC(*)=O]

108. The at least one compound according to any one of claims 1-102, wherein L is chosen from 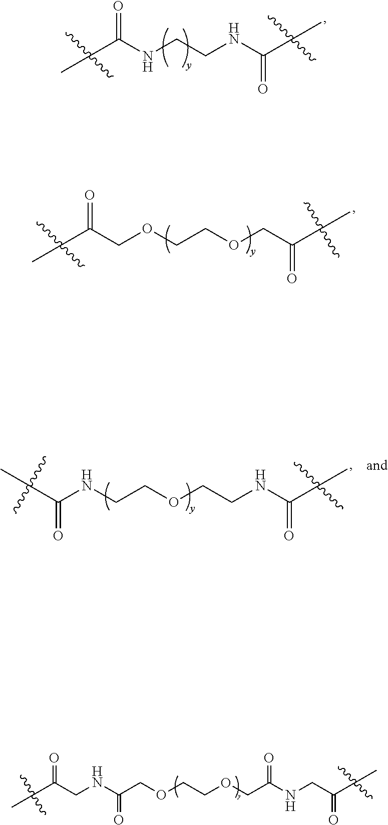
groups
wherein y is chosen from integers ranging from 0 to 250.

109. The at least one compound according to any one of claims 1-102, wherein L is chosen from 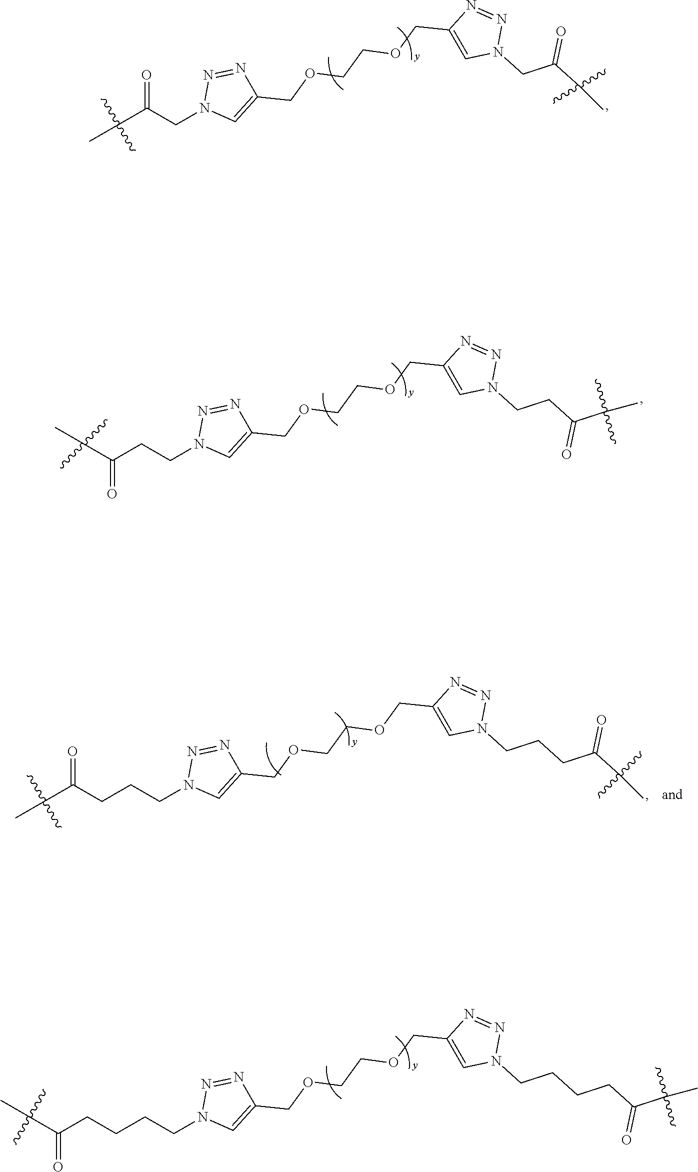
groups
wherein y is chosen from integers ranging from 0 to 250.

110. The at least one compound according to any one of claims 1-102, wherein L is chosen from

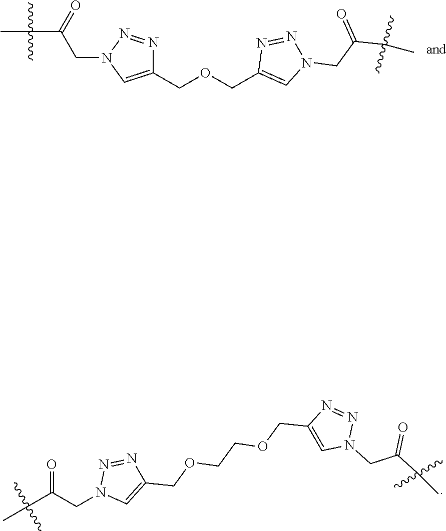 [SMILES: *C(=O)Cn1cc(COCc2cn(CC(*)=O)nn2)nn1 ; *C(=O)Cn1cc(COCCOCc2cn(CC(*)=O)nn2)nn1]

111. The at least one compound according to any one of claims 1-102, wherein L is chosen from

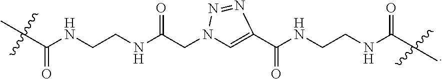 [SMILES: *C(=O)NCCNC(=O)Cn1cc(C(=O)NCCNC(*)=O)nn1]

112. The at least one compound according to an one of claims 1-102 wherein L is 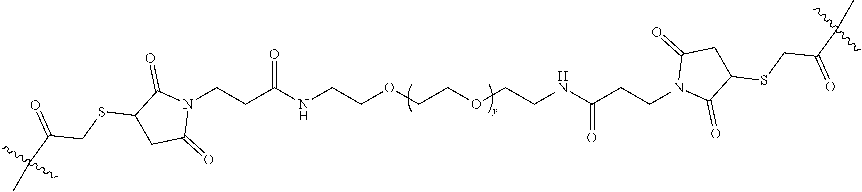
wherein y is chosen from integers ranging from 0 to 250.

113. The at least one compound according to any one of claims 99, 103-106, 108, 109, and 112, wherein each y is identical and chosen from integers ranging from 0 to 25.

114. The at least one compound according to claim 113, wherein each y is identical and chosen from integers ranging from 0 to 15.

115. The at least one compound according to claim 113, wherein each y is identical and chosen from integers ranging from 0 to 5.

116. The at least one compound according to claim 113, wherein each y is 14.

117. The at least one compound according to claim 113, wherein each y is 10.

118. The at least one compound according to claim 113, wherein each y is 3.

119. The at least one compound according to claim 113, wherein each y is 2.

120. The at least one compound according to claim 113, wherein each y is 1.

121. The at least one compound according to claim 113, wherein each y is 0.

122. The at least one compound according to any preceding claim, wherein each R¹ is identical, each R² is identical, each R³ is identical, each R⁴ is identical, each R⁵ is identical, and each X is identical.

123. A composition comprising at least one compound according to any preceding claim and at least one additional pharmaceutically acceptable ingredient.

124. A method for treatment and/or prevention of at least one disease, disorder, and/or condition where inhibition of E-selectin, galectin-3, and/or CXCR4 chemokine receptor inhibitor mediated functions is useful, the method comprising administering to a subject in need thereof an effective amount of at least one compound of any one of claims 1-122.

125. A method for treatment and/or prevention of at least one inflammatory disease, disorder, and/or condition, the method comprising administering to a subject in need thereof an effective amount of at least one compound of any one of claims 1-122.

126. A method for treatment and/or prevention of cancer, the method comprising administering to a subject in need thereof an effective amount of at least one compound of any one of claims 1-122.

127. The method according to claim 126, wherein the cancer is chosen from solid tumor cancers.

128. The method according to claim 126, wherein the cancer is chosen from bone cancers, colorectal cancers, and pancreatic cancers.

129. The method according to claim 126, wherein the cancer is chosen from liquid tumor cancers.

130. The method according to claim 126, wherein the cancer is chosen from acute myelogenous leukemia, acute lymphoblastic leukemia, chronic myelogenous leukemia, and multiple myeloma.

131. A method for treatment and/or prevention of cancer, the method comprising administering to a subject in need thereof (a) an effective amount of at least one compound of any one of claims 1-122 and (b) at least one of therapy chosen from (i) chemotherapy and (ii) radiotherapy.

132. A method for treatment and/or prevention of metastasis of cancer cells, the method comprising administering to a subject in need thereof an effective amount of at least one compound of any one of claims 1-122.

133. A method for inhibiting infiltration of cancer cells into the liver, lymph nodes, lung, bone, and/or bone marrow, the method comprising administering to a subject in need thereof an effective amount of at least one compound of any one of claims 1-122.

134. A method for enhancing hematopoietic stem cell survival, the method comprising administering to a subject in need thereof an effective amount of at least one compound of any one of claims 1-122.

135. The method according to claim 134, wherein the subject has cancer and has received or will receive chemotherapy and/or radiotherapy.

136. A method for mobilizing cells from the bone marrow, the method comprising administering to a subject in need thereof an effective amount of at least one compound of any one of claims 1-122.

137. The method according to claim 136, wherein the cells are chosen from hematopoietic cells and tumor cells.

138. A method for treatment and/or prevention of mucositis, the method comprising administering to a subject in need thereof an effective amount of at least one compound of any one of claims 1-122.

139. The method according to claim 138, wherein the mucositis is chosen from oral mucositis, esophageal mucositis, and gastrointestinal mucositis.

140. The method according to claim 138, wherein the subject is afflicted with head and neck, breast, lung, ovarian, prostate, lymphatic, leukemic, and/or gastrointestinal cancer.

141. A method for treatment and/or prevention of thrombosis, the method comprising administering to a subject in need thereof an effective amount of at least one compound of any one of claims 1-122.

142. A method for treatment and/or prevention of at least one cardiovascular disease or complications associated therewith, the method comprising administering to a subject in need thereof an effective amount of at least one compound of any one of claims 1-122.

143. The method according to claim 142, wherein the at least one cardiovascular disease is chosen from atherosclerosis and myocardial infarction.

144. A method of inhibiting rejection of a transplanted tissue in a subject, wherein said subject is a recipient of the transplanted tissue, the method comprising administering to a subject in need thereof an effective amount of at least one compound of any one of claims 1-122.

145. A method for treatment and/or prevention of graft versus host disease or complications associated therewith, the method comprising administering to a subject in need thereof an effective amount of at least one compound of any one of claims 1-122.

146. A method for treatment and/or prevention of pathological angiogenesis, the method comprising administering to a subject in need thereof an effective amount of at least one compound of any one of claims 1-122.

147. The method according to claim 146, wherein the pathological angiogenesis occurs in the eye.

148. The method according to claim 146, wherein the pathological angiogenesis occurs in a subject with cancer.

149. A method for treatment and/or prevention of an epileptic syndrome, the method comprising administering to a subject in need thereof an effective amount of at least one compound of any one of claims 1-122.

150. A method for treatment and/or prevention of neurodegeneration, the method comprising administering to a subject in need thereof an effective amount of at least one compound of any one of claims 1-122.

151. The method according to claim 150, wherein the neurodegenerative disease is α-synucleinopathy.

152. A method for treatment and/or prevention of fibrosis, the method comprising administering to a subject in need thereof an effective amount of at least one compound of any one of claims 1-122.

153. The method according to claim 152, wherein the fibrosis is pulmonary fibrosis.

154. The method according to claim 152, wherein the fibrosis is cardiac fibrosis.

155. A method for treatment and/or prevention of liver disorders or complications associated therewith, the method comprising administering to a subject in need thereof an effective amount of at least one compound of any one of claims 1-122.

156. The method according to claim 155, wherein the liver disorder is nonalcoholic steatohepatitis.

157. A method for for treatment and/or prevention of sickle cell disease or complications associated therewith comprising administering to a subject in need thereof an effective amount of at least one compound of any one of claims 1-122.

158. A method for treatment and/or prevention of vaso-occlusive crisis comprising administering to a subject in need thereof an effective amount of at least one compound of any one of claims 1-122.

159. A method for treatment and/or prevention of sinusoidal obstruction syndrome or complications associated therewith comprising administering to a subject in need thereof an effective amount of at least one compound of any one of claims 1-122.

160. A method for treatment and/or prevention of a cancer of the blood and complications associated therewith comprising administering to a subject in need thereof an effective amount of at least one compound of any one of claims 1-122.

161. The method of claim 160, wherein the cancer of the blood is chosen from acute myelogenous leukemia, acute lymphoblastic leukemia, chronic myelogenous leukemia, and multiple myeloma.

162. A method for treating epilepsy comprising administering to a subject in need thereof an effective amount of at least one compound of any one of claims 1-122.
